(19) United States
(12) Patent Application Publication
(10) Pub. No.: US 2012/0101083 A1
(43) Pub. Date: Apr. 26, 2012

(54) S1P1 AGONISTS COMPRISING A BICYCLIC N-CONTAINING RING

(75) Inventors: James Matthew Bailey, Stevenage, PA (US); Rino Antonio Bit, Stevenage (GB); Emmanuel Hubert Demont, Stevenage (GB); Lee Andrew Harrison, Stevenage (GB); Katherine Louise Jones, Stevenage (GB); Christian Alan Paul Smethurst, Harlow (GB); Jason Witherington, Stevenage (GB)

(73) Assignee: GLAXO GROUP LIMITED, Greenford, Middlesex (GB)

(21) Appl. No.: 13/379,059

(22) PCT Filed: Jun. 17, 2010
(86) PCT No.: PCT/EP2010/058515

§ 371 Date: Dec. 19, 2011

(30) Foreign Application Priority Data
Jun. 19, 2009 (GB) 0910674.1

(51) Int. Cl. A61K 31/55; C07D 417/10; C07D 413/14; A61K 31/4725; A61P 25/00; A61P 29/00; A61P 11/06; A61P 19/02; A61P 17/06; A61P 35/00; A61P 35/04; A61P 9/00; A61P 31/12; A61P 3/10; C07D 413/10

(52) U.S. Cl. 514/210.18; 546/145; 540/594; 514/307; 514/217.01

(57) ABSTRACT

The present invention relates to novel compounds of formula (I) having S1P1 agonist activity, processes for their preparation, pharmaceutical compositions containing them and their use in the treatment of various disorders.

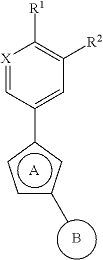

The present invention relates to novel compounds having pharmacological activity, processes for their preparation, pharmaceutical compositions containing them and their use in the treatment of various disorders.

Sphingosine 1-phosphate (S1P) is a bioactive lipid mediator formed by the phosphorylation of sphingosine by sphingosine kinases and is found; in high levels in the blood. It is produced and secreted by a number of cell types, including those of hematopoietic origin such as platelets and mast cells (Okamoto et al 1998 J Biol Chem 273(42):27104; Sanchez and Hla 2004, J Cell Biochem 92:913). It has a wide range of biological actions, including regulation of cell proliferation, differentiation, motility, vascularisation, and activation of inflammatory cells and platelets (Pyne and Pyne 2000, Biochem J. 349: 385). Five subtypes of S1P responsive receptor have been described, S1P1 (Edg-1), S1P2 (Edg-5), S1P3 (Edg-3), S1P4 (Edg-6), and S1P5 (Edg-8), forming part of the G-protein coupled endothelial differentiation gene family of receptors (Chun et al 2002 Pharmacological Reviews 54:265, Sanchez and Hla 2004 J Cellular Biochemistry, 92:913). These 5 receptors show differential mRNA expression, with S1P1-3 being widely expressed, S1P4 expressed on lymphoid and hematopoietic tissues and S1P5 primarily in brain and to a lower degree in spleen. They signal via different subsets of G proteins to promote a variety of biological responses (Kluk and Hla 2002 Biochem et Biophysica Acta 1582:72, Sanchez and Hla 2004, J Cellular Biochem 92:913).

Proposed roles for the S1P1 receptor include lymphocyte trafficking, cytokine induction/suppression and effects on endothelial cells (Rosen and Goetzl 2005 Nat Rev Immunol. 5:560). Agonists of the S1P1 receptor have been used in a number of autoimmune and transplantation animal models, including Experimental Autoimmune Encephalomelitis (EAE) models of MS, to reduce the severity of the induced disease (Brinkman et al 2003 JBC 277:21453; Fujino et al 2003 J Pharmacol Exp Ther 305:70; Webb et al 2004 J Neuroimmunol 153:108; Rausch et al 2004 J Magn Reson Imaging 20:16). This activity is reported to be mediated by the effect of S1P1 agonists on lymphocyte circulation through the lymph system. Treatment with S1P1 agonists results in the sequestration of lymphocytes within secondary lymphoid organs such as the lymph nodes, inducing a reversible peripheral lymphopoenia in animal models (Chiba et al 1998, J Immunology 160:5037, Forrest et al 2004 J Pharmacol Exp Ther 309:758; Sanna et al 2004 JBC 279:13839). Published data on agonists suggests that compound treatment induces loss of the S1P1 receptor from the cell surface via internalisation (Graler and Goetzl 2004 FASEB J 18:551; Matloubian et al 2004 Nature 427:355; Jo et al 2005 Chem Biol 12:703) and it is this reduction of S1P1 receptor on immune cells which contributes to the reduction of movement of T cells from the lymph nodes back into the blood stream.

S1P1 gene deletion causes embryonic lethality. Experiments to examine the role of the S1P1 receptor in lymphocyte migration and trafficking have included the adoptive transfer of labelled S1P1 deficient T cells into irradiated wild type mice. These cells showed a reduced egress from secondary lymphoid organs (Matloubian et al 2004 Nature 427:355).

S1P1 has also been ascribed a role in endothelial cell junction modulation (Allende et al 2003 102:3665, Blood Singelton et al 2005 FASEB J 19:1646). With respect to this endothelial action, S1P1 agonists have been reported to have an effect on isolated lymph nodes which may be contributing to a role in modulating immune disorders. S1P1 agonists caused a closing of the endothelial stromal ‘gates’ of lymphatic sinuses which drain the lymph nodes and prevent lymphocyte egress (Wei wt al 2005, Nat. Immunology 6:1228).

The immunosuppressive compound FTY720 (JP11080026-A) has been shown to reduce circulating lymphocytes in animals and man, have disease modulating activity in animal models of immune disorders and reduce remission rates in relapsing remitting Multiple Sclerosis (Brinkman et al 2002 JBC 277:21453, Mandala et al 2002 Science 296:346, Fujino et al 2003 J Pharmacology and Experimental Therapeutics 305:45658, Brinkman et al 2004 American J Transplantation 4:1019, Webb et al 2004 J Neuroimmunology 153:108, Morris et al 2005 EurJ Immunol 35:3570, Chiba 2005 Pharmacology and Therapeutics 108:308, Kahan et al 2003, Transplantation 76:1079, Kappos et al 2006 New Eng J Medicine 335:1124). This compound is a prodrug that is phosphorylated in vivo by sphingosine kinases to give a molecule that has agonist activity at the S1P1, S1P3, S1P4 and S1P5 receptors. Clinical studies have demonstrated that treatment with FTY720 results in bradycardia in the first 24 hours of treatment (Kappos et al 2006 New Eng J Medicine 335:1124). The bradycardia is thought to be due to agonism at the S1P3 receptor, based on a number of cell based and animal experiments. These include the use of S1P3 knock-out animals which, unlike wild type mice, do not demonstrate bradycardia following FTY720 administration and the use of S1P1 selective compounds. (Hale et al 2004 Bioorganic & Medicinal Chemistry Letters 14:3501, Sanna et al 2004 JBC 279:13839, Koyrakh et al 2005 American J Transplantation 5:529)

Hence, there is a need for S1P1 receptor agonist compounds with selectivity over S1P3 which might be expected to show a reduced tendency to induce bradycardia.

The following patent applications describe oxadiazole derivatives as S1P1 agonists: WO03/105771, WO05/058848, WO06/047195, WO06/100633, WO06/115188, WO06/131336, WO07/024,922 and WO07/116,866.

The following patent applications describe tetrahydroisoquinolinyl-oxadiazole derivatives as S1P receptor agonists: WO06/064757, WO06/001463, WO04/113330.

WO08/064,377 describes benzocycloheptyl analogs having S1P1 receptor activity.

A structurally novel class of compounds has now been found; which provides agonists of the S1P1 receptor.

The present invention provides compounds of formula (I) or a pharmaceutically acceptable salt thereof thereof:

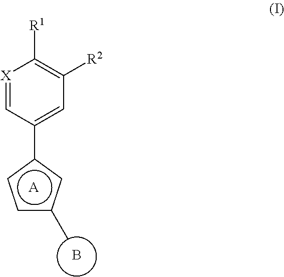

### X is CH or N;

R¹ is OR³, NHR⁴, R⁵, NR⁶R⁷, R⁸ or optionally fluorinated C₍₃₋₆₎cycloalkyl;
R² is hydrogen, halogen, cyano, trifluoromethyl, C₍₁₋₂₎alkoxy and C₍₁₋₃₎alkyl optionally substituted by halogen;
R³ and R⁴ are C₍₁₋₅₎alkyl optionally interrupted by O and optionally substituted by F or (CH₂)₍₀₋₁₎C₍₃₋₅₎cycloalkyl optionally substituted by F;
R⁵ is C₍₁₋₆₎alkyl optionally substituted by F;
R⁶ and R⁷ are independently selected from C₍₁₋₅₎alkyl optionally interrupted by O and optionally substituted by F and optionally fluorinated C₍₃₋₅₎cycloalkyl with the proviso that the combined number of carbon atoms in R⁶ and R⁷ does not exceed 6;
R⁸ is a 3 to 6 membered, nitrogen-containing heterocyclyl ring optionally substituted by F selected from aziridinyl, azetidinyl, pyrrolidinyl, piperidinyl and morpholinyl, all attached via the nitrogen atom;
A is a 5-membered heterocyclic ring selected from the following:

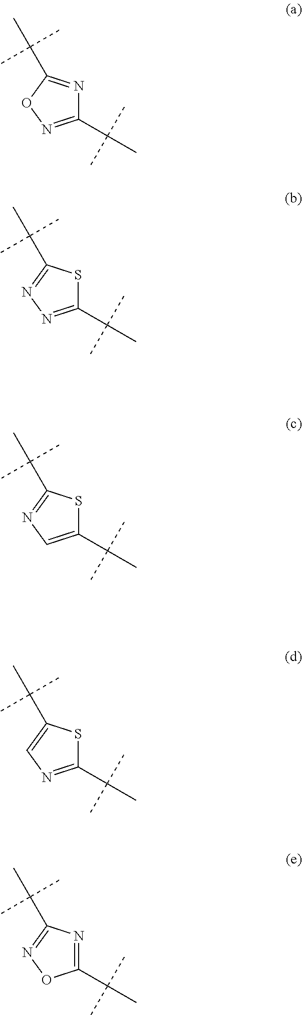

B is a bicyclic ring selected from the following:

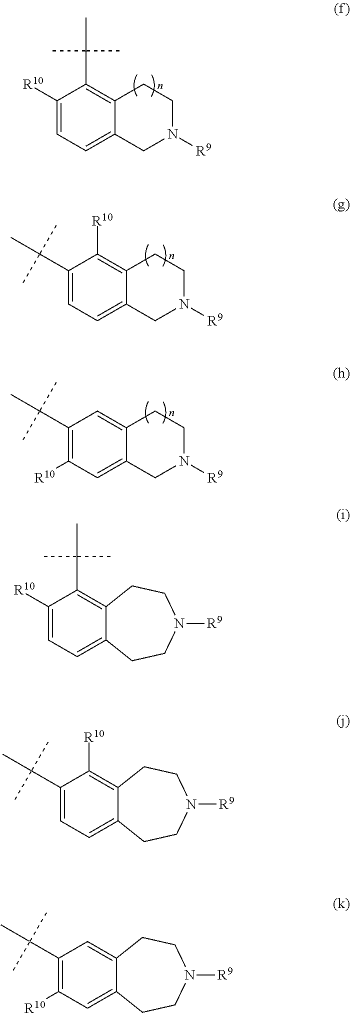

R⁹ is C₍₁₋₄₎alkyl substituted by at least one OH and optionally interrupted by O, C₍₁₋₄₎alkyl interrupted by O, C₍₂₋₄₎alkyl substituted by SO₂C₍₁₋₃₎alkyl, C₍₁₋₄₎alkylCONR¹¹R¹², C₍₂₋₄₎alkylNR¹³CONR¹¹R¹², C₍₂₋₄₎alkylNR¹³COOR¹², C₍₂₋₄₎alkylOCONR¹¹R¹², C₍₂₋₄₎alkylNR¹³COR¹² or COC₍₁₋₄₎alkylNR¹¹R¹²;
when R⁹ is COC₍₁₋₄₎alkylNR¹¹R¹² the alkyl chain may be optionally substituted by C₍₁₋₃₎alkylOH or interrupted by O; when R⁹ is C₍₁₋₄₎alkylCONR¹¹R¹², C₍₂₋₄₎alkylNR¹³COOR¹², C₍₂₋₄₎alkylOCONR¹¹R¹², C₍₂₋₄₎alkylNR¹³COR¹² and comprises an alkyl chain of at least two carbon atoms at the point of attachment to the B ring it may be optionally substituted by halogen, OC₍₁₋₃₎alkyl or OH;
R¹⁰ is hydrogen or C₍₁₋₃₎alkyl optionally substituted by halogen;
R¹¹, R¹² and R¹³ are independently selected from hydrogen or C₍₁₋₃₎alkyl optionally substituted by F or hydroxyl and optionally interrupted by O;
R¹¹ and R¹² together with the nitrogen atom to which they are attached may be linked to form a 4-6 membered heterocyclyl ring, wherein the 4- to 6-membered heterocyclyl ring optionally contains an oxygen atom and is optionally substituted by one or two substituents independently selected from F and OH;
R¹² and R¹³, together with the atoms to which they are attached may be linked to form an optionally unsaturated 5-7 membered heterocyclyl ring, wherein the 5- to 7-membered heterocyclyl ring optionally contains an oxygen atom and is optionally substituted by one or two substituents independently selected from F and OH; and n is 0, 1 or 2.

In one embodiment X is CH. In another embodiment X is N.

In one embodiment R¹ is OR³.

In one embodiment R³ is isopropyl.

In one embodiment R² is chloro or cyano.

In one embodiment A is (a) or (b).

In one embodiment B is (f), (g), (i) or (j).

In one embodiment R⁹ is C₍₁₋₄₎alkyl substituted by at least one OH and optionally interrupted by O, C₍₁₋₄₎alkyl interrupted by O or C₍₂₋₄₎alkyl substituted by SO₂C₍₁₋₃₎alkyl.

In one embodiment R¹⁰ is hydrogen or methyl.

In one embodiment R¹¹ and R¹² are independently selected from hydrogen, methyl

In one embodiment n is 1.

In one embodiment

### X is CH or N;

### R¹ is OR³;

R³ is isopropyl;
R² is chloro or cyano;

### A is (a) or (b);

### B is (f), (g), (i) or a);

R⁹ is C₍₁₋₄₎alkyl substituted by at least one OH and optionally interrupted by O, C₍₁₋₄₎alkyl interrupted by O or C₍₂₋₄₎alkyl substituted by SO₂C₍₁₋₃₎alkyl;
R¹⁰ is hydrogen or methyl; and
n is 1.

In one embodiment X is CH. In another embodiment X is N.

In one embodiment R¹ is OR³, NHR⁴ or R⁸.

In one embodiment R³ is C₍₂₋₃₎alkyl optionally substituted by two or three fluoro. In another embodiment R³ is isopropyl, propyl and ethyl, each optionally substituted by fluoro.

In one embodiment R² is chloro or cyano.

In one embodiment R⁴ is C₍₃₎alkyl. In another embodiment R⁴ is isopropyl or propyl.

In one embodiment R⁸ is azetidinyl or pyrrolidinyl each optionally substituted by fluoro.

In one embodiment A is (a) or (b).

In one embodiment B is (f), (g), (i) or (j).

In one embodiment R⁹ is C₍₁₋₄₎alkyl substituted by at least one OH, C₍₁₋₄₎alkyl interrupted by O, C₍₁₋₄₎alkylCONR¹¹R¹², C₍₂₋₄₎alkylNR¹³CONR¹¹R¹², C₍₂₋₄₎alkylNR¹³COOR¹², C₍₂₋₄₎alkylNR¹³COR¹² or COC₍₁₋₄₎alkylNR¹¹R¹². In another embodiment R⁹ is C₍₂₋₃₎alkyl substituted by one or two OH, C₍₃₎alkyl interrupted by O, C₍₁₋₃₎alkylCONR¹¹R¹², C₍₂₎alkyl NR¹³CONR¹¹R¹², C₍₂₋₃₎alkylNR¹³COOR¹², C₍₃₎alkylNR¹³COR¹² or COC₍₁₋₃₎alkylNR¹¹R¹².

In one embodiment, when R⁹ is COC₍₁₋₄₎alkylNR¹¹R¹² the alkyl chain may be optionally substituted by C₍₂₎alkylOH.

In one embodiment R¹⁰ is hydrogen or C₍₁₋₃₎alkyl.

In one embodiment R¹¹ is hydrogen or C₍₁₋₃₎alkyl optionally substituted by one or more fluoro or on separate C atoms by one or more OH.

In one embodiment R¹² is hydrogen or C₍₁₋₃₎alkyl optionally substituted by one or more fluoro or on separate C atoms by one or more OH.

In another embodiment R¹¹ and R¹² together with the nitrogen atom to which they are attached may be linked to form azetidinyl or morpholinyl each optionally substituted by one or two substituents independently selected from F and OH. In a further embodiment R¹¹ and R¹² together with the nitrogen atom to which they are attached may be linked to form azetidinyl or morpholinyl each optionally substituted by one substituent selected from F and OH.

In one embodiment R¹³ is hydrogen.

In one embodiment n is 1.

In one embodiment

### X is CH or N;

### R¹ is OR³;

R³ is isopropyl;
R² is chloro or cyano;

### A is (a) or (b);

### B is (f), (g), (i) or a);

R⁹ is C₍₁₋₄₎alkyl substituted by at least one OH and optionally interrupted by O, C₍₁₋₄₎alkyl interrupted by O or C₍₂₋₄₎alkyl substituted by SO₂C₍₁₋₃₎alkyl;
R¹⁹ is hydrogen or methyl; and
n is 1.

The term “alkyl” as a group or part of a group e.g. alkoxy or hydroxyalkyl refers to a straight or branched alkyl group in all isomeric forms. The term “C₍₁₋₆₎ alkyl” refers to an alkyl group, as defined above, containing at least 1, and at most 6 carbon atoms Examples of such alkyl groups include methyl, ethyl, propyl, iso-propyl, n-butyl, iso-butyl, sec-butyl, or tert-butyl. Examples of such alkoxy groups include methoxy, ethoxy, propoxy, iso-propoxy, butoxy, iso-butoxy, sec-butoxy and tert-butoxy.

Suitable C₍₃₋₆₎cycloalkyl groups include cyclopropyl, cyclobutyl, cyclopentyl and cyclohexyl.

As used herein, the term “halogen” refers to fluorine (F), chlorine (Cl), bromine (Br), or iodine (I) and the term “halo” refers to the halogen: fluoro (—F), chloro (—Cl), bromo(—Br) and iodo(—I).

The term “substituted” includes the implicit provision that substitution be in accordance with the permitted valence of the substituted atom and the substituent and that the substitution results in a stable compound (i.e. one that does not spontaneously undergo transformation such as by rearrangement, cyclization, or elimination). In certain embodiments, a single atom may be substituted with more than one substituent as long as such substitution is in accordance with the permitted valence of the atom. In certain embodiments, alkyl groups optionally substituted by F or OH may be multiply substituted on multiple carbon atoms.

In certain of the compounds of formula (I), dependent upon the nature of the substituent there are chiral carbon atoms and therefore compounds of formula (I) may exist as stereoisomers. The invention extends to all optical isomers such as stereoisomeric forms of the compounds of formula (I) including enantiomers, diastereoisomers and mixtures thereof, such as racemates. The different stereoisomeric forms may be separated or resolved one from the other by conventional methods or any given isomer may be obtained by conventional stereoselective or asymmetric syntheses.

Certain of the compounds herein can exist in various tautomeric forms and it is to be understood that the invention encompasses all such tautomeric forms.

It is understood that certain compounds of the invention contain both acidic and basic groups and may therefore exist as zwitterions at certain pH values.

Suitable compounds of the invention are:
3-[6-(5-{3-Cyano-4-[(1-methylethyl)oxy]phenyl}-1,2,4-oxadiazol-3-yl)-5-methyl-3,4-dihydro-2(1H)-isoquinolinyl]propanamide
3-[6-(5-{3-Cyano-4-[(1-methylethyl)oxy]phenyl}-1,2,4-oxadiazol-3-yl)-5-methyl-3,4-dihydro-2(1H)-isoquinolinyl]-N,N-dimethylpropanamide
2-[6-(5-{3-Cyano-4-[(1-methylethyl)oxy]phenyl}-1,2,4-oxadiazol-3-yl)-5-methyl-3,4-dihydro-2(1H)-isoquinolinyl]acetamide
4-[6-(5-{3-Cyano-4-[(1-methylethyl)oxy]phenyl}-1,2,4-oxadiazol-3-yl)-5-methyl-3,4-dihydro-2(1H)-isoquinolinyl]butanamide
4-[6-(5-{3-Cyano-4-[(1-methylethyl)oxy]phenyl}-1,2,4-oxadiazol-3-yl)-5-methyl-3,4-dihydro-2(1H)-isoquinolinyl]-N-methylbutanamide
5-{3-[2-(2,3-Dihydroxypropyl)-5-methyl-1,2,3,4-tetrahydro-6-isoquinolinyl]-1,2,4-oxadiazol-5-yl}-2-[(1-methylethyl)oxy]benzonitrile
5-{3-[2-(2-Hydroxyethyl)-5-methyl-1,2,3,4-tetrahydro-6-isoquinolinyl}-1,2,4-oxadiazol-5-yl]-2-[(1-methylethyl)oxy]benzonitrile
5-{3-[2-(3-Hydroxypropyl)-5-methyl-1,2,3,4-tetrahydro-6-isoquinolinyl]-1,2,4-oxadiazol-5-yl}-2-[(1-methylethyl)oxy]benzonitrile
2-[(1-Methylethyl)oxy]-5-(3-{5-methyl-2-[2-(methyloxy)ethyl]-1,2,3,4-tetrahydro-6-isoquinolinyl}-1,2,4-oxadiazol-5-yl)benzonitrile
3-[6-(5-{3-Cyano-4-[(1-methylethyl)oxy]phenyl}-1,3,4-thiadiazol-2-yl)-5-methyl-3,4-dihydro-2(1H)-isoquinolinyl]propanamide
3-[6-(5-{3-Cyano-4-[(1-methylethyl)oxy]phenyl}-1,3,4-thiadiazol-2-yl)-5-methyl-3,4-dihydro-2(1H)-isoquinolinyl]-N-methylpropanamide
3-[6-(5-{3-Cyano-4-[(1-methylethyl)oxy]phenyl}-1,3,4-thiadiazol-2-yl)-5-methyl-3,4-dihydro-2(1H)-isoquinolinyl]-N,N-dimethylpropanamide
4-[6-(5-{3-Cyano-4-[(1-methylethyl)oxy]phenyl}-1,3,4-thiadiazol-2-yl)-5-methyl-3,4-dihydro-2(1H)-isoquinolinyl]butanamide
4-[6-(5-{3-Cyano-4-[(1-methylethyl)oxy]phenyl}-1,3,4-thiadiazol-2-yl)-5-methyl-3,4-dihydro-2(1H)-isoquinolinyl]-N-methylbutanamide
5-{5-[2-(2-Hydroxyethyl)-5-methyl-1,2,3,4-tetrahydro-6-isoquinolinyl]-1,3,4-thiadiazol-2-yl}-2-[(1-methylethyl)oxy]benzonitrile
5-{5-[2-(3-Hydroxypropyl)-5-methyl-1,2,3,4-tetrahydro-6-isoquinolinyl]-1,3,4-thiadiazol-2-yl}-2-[(1-methylethyl)oxy]benzonitrile
2-[(1-Methylethyl)oxy]-5-(5-{5-methyl-2-[2-(methyloxy)ethyl]-1,2,3,4-tetrahydro-6-isoquinolinyl}-1,3,4-thiadiazol-2-yl)benzonitrile
5-{5-[2-(2,3-Dihydroxypropyl)-5-methyl-1,2,3,4-tetrahydro-6-isoquinolinyl]-1,3,4-thiadiazol-2-yl}-2-[(1-methylethyl)oxy]benzonitrile
5-{3-[3-(2-Hydroxyethyl)-2,3,4,5-tetrahydro-1H-3-benzazepin-7-yl]-1,2,4-oxadiazol-5-yl}-2-[(1-methylethyl)oxy]benzonitrile
5-{3-[3-(3-Hydroxypropyl)-2,3,4,5-tetrahydro-1H-3-benzazepin-7-yl]-1,2,4-oxadiazol-5-yl}-2-[(1-methylethyl)oxy]benzonitrile
2-[(1-Methylethyl)oxy]-5-(3-{3-[2-(methyloxy)ethyl]-2,3,4,5-tetrahydro-1H-3-benzazepin-7-yl}-1,2,4-oxadiazol-5-yl)benzonitrile
3-[7-(5-{3-Cyano-4-[(1-methylethyl)oxy]phenyl}-1,2,4-oxadiazol-3-yl)-1,2,4,5-tetrahydro-3H-3-benzazepin-3-yl]propanamide
3-[7-(5-{3-Cyano-4-[(1-methylethyl)oxy]phenyl}-1,2,4-oxadiazol-3-yl)-1,2,4,5-tetrahydro-3H-3-benzazepin-3-yl]-N,N-dimethylpropanamide
4-[7-(5-{3-Cyano-4-[(1-methylethyl)oxy]phenyl}-1,2,4-oxadiazol-3-yl)-1,2,4,5-tetrahydro-3H-3-benzazepin-3-yl]-N-methylbutanamide
4-[7-(5-{3-Cyano-4-[(1-methylethyl)oxy]phenyl}-1,2,4-oxadiazol-3-yl)-1,2,4,5-tetrahydro-3H-3-benzazepin-3-yl]butanamide
4-[7-(5-{3-Cyano-4-[(1-methylethyl)oxy]phenyl}-1,2,4-oxadiazol-3-yl)-1,2,4,5-tetrahydro-3H-3-benzazepin-3-yl]-N,N-dimethylbutanamide
5-{3-[3-(2,3-Dihydroxypropyl)-2,3,4,5-tetrahydro-1H-3-benzazepin-7-yl]-1,2,4-oxadiazol-5-yl}-2-[(1-methylethyl)oxy]benzonitrile
5-{5-[3-(2-Hydroxyethyl)-2,3,4,5-tetrahydro-1H-3-benzazepin-7-yl]-1,3,4-thiadiazol-2-yl}-2-[(1-methylethyl)oxy]benzonitrile
5-{5-[3-(3-Hydroxypropyl)-2,3,4,5-tetrahydro-1H-3-benzazepin-7-yl]-1,3,4-thiadiazol-2-yl}-2-[(1-methylethyl)oxy]benzonitrile
2-[(1-Methylethyl)oxy]-5-(5-{3-[2-(methyloxy)ethyl]-2,3,4,5-tetrahydro-1H-3-benzazepin-7-yl}-1,3,4-thiadiazol-2-yl)benzonitrile
2-[6-(5-{3-Cyano-4-[(1-methylethyl)oxy]phenyl}-1,2,4-oxadiazol-3-yl)-1,2,4,5-tetrahydro-3H-3-benzazepin-3-yl]) acetamide
3-[6-(5-{3-Cyano-4-[(1-methylethyl)oxy]phenyl}-1,2,4-oxadiazol-3-yl)-1,2,4,5-tetrahydro-3H-3-benzazepin-3-yl]propanamide
5-{3-[3-(3-Hydroxypropyl)-2,3,4,5-tetrahydro-1H-3-benzazepin-6-yl]-1,2,4-oxadiazol-5-yl}-2-[(1-methylethyl)oxy]benzonitrile
5-{3-[3-(2-Hydroxyethyl)-2,3,4,5-tetrahydro-1H-3-benzazepin-6-yl]-1,2,4-oxadiazol-5-yl}-2-[(1-methylethyl)oxy]benzonitrile
5-{3-[3-(2,3-Dihydroxypropyl)-2,3,4,5-tetrahydro-1H-3-benzazepin-6-yl]-1,2,4-oxadiazol-5-yl}-2-[(1-methylethyl)oxy]benzonitrile
5-{5-[2-(3-Hydroxypropyl)-1,2,3,4-tetrahydro-5-isoquinolinyl]-1,3,4-thiadiazol-2-yl}-2-[(1-methylethyl)oxy]benzonitrile
2-[(1-Methylethyl)oxy]-5-(5-{2-[2-(methyloxy)ethyl]-1,2,3,4-tetrahydro-5-isoquinolinyl}-1,3,4-thiadiazol-2-yl)benzonitrile
5-{5-[2-(2-hydroxyethyl)-1,2,3,4-tetrahydro-5-isoquinolinyl]-1,3,4-thiadiazol-2-yl}-2-[(1-methylethyl)oxy]benzonitrile
5-[4-(2-β-Alanyl-5-methyl-1,2,3,4-tetrahydro-6-isoquinolinyl)-1,2,4-oxadiazol-5-yl]-2-[(1-methylethyl)oxy]benzonitrile
2-[(1-Methylethyl)oxy]-5-{3-[5-methyl-2-(N-methyl-□-alanyl)-1,2,3,4-tetrahydro-6-isoquinolinyl]-1,2,4-oxadiazol-5-yl}benzonitrile
5-{3-[2-(4-Aminobutanoyl)-5-methyl-1,2,3,4-tetrahydro-6-isoquinolinyl]-1,2,4-oxadiazol-5-yl}-2-[(1-methylethyl)oxy]benzonitrile
5-[4-(2-Glycyl-5-methyl-1,2,3,4-tetrahydro-6-isoquinolinyl)-1,2,4-oxadiazol-5-yl]-2-[(1-methylethyl)oxy]benzonitrile
3-[6-(5-{3-cyano-4-[(1-methylethyl)oxy]phenyl}-1,2,4-oxadiazol-3-yl)-5-methyl-3,4-dihydro-2(1H)-isoquinolinyl]-N-methylpropanamide
4-[7-(5-{3-cyano-4-[(1-methylethyl)oxy]phenyl}-1,2,4-oxadiazol-3-yl)-1,2,4,5-tetrahydro-3H-3-benzazepin-3-yl]-N-ethylbutanamide
3-[5-(5-{3-cyano-4-[(1-methylethyl)oxy]phenyl}-1,3,4-thiadiazol-2-yl)-3,4-dihydro-2(1H)-isoquinolinyl]-N-methylpropanamide
3-[5-(5-{3-cyano-4-[(1-methylethyl)oxy]phenyl}-1,3,4-thiadiazol-2-yl)-3,4-dihydro-2(1H)-isoquinolinyl]-N,N-dimethylpropanamide
3-[6-(5-{3-cyano-4-[(1-methylethyl)oxy]phenyl}-1,3,4-thiadiazol-2-yl)-3,4-dihydro-2(1H)-isoquinolinyl]propanamide
5-[3-(3-glycyl-2,3,4,5-tetrahydro-1H-3-benzazepin-7-yl)-1,2,4-oxadiazol-5-yl]-2-[(1-methylethyl)oxy]benzonitrile trifluoroacetate
5-{3-[3-(2,3-dihydroxypropyl)-2,3,4,5-tetrahydro-1H-3-benzazepin-7-yl]-1,2,4-oxadiazol-5-yl}-2-[(1-methylethyl)amino]benzonitrile
5-{3-[3-(2,3-dihydroxypropyl)-2,3,4,5-tetrahydro-1H-3-benzazepin-7-yl]-1,2,4-oxadiazol-5-yl}-2-(propylamino)benzonitrile
5-{3-[3-(2,3-dihydroxypropyl)-2,3,4,5-tetrahydro-1H-3-benzazepin-7-yl]-1,2,4-oxadiazol-5-yl}-2-(propyloxy)benzonitrile
5-[3-(3-glycyl-2,3,4,5-tetrahydro-1H-3-benzazepin-7-yl)-1,2,4-oxadiazol-5-yl]-2-[(1-methylethyl)amino]benzonitrile
5-[3-(3-glycyl-2,3,4,5-tetrahydro-1H-3-benzazepin-7-yl)-1,2,4-oxadiazol-5-yl]-2-(propylamino)benzonitrile
2-{[2-fluoro-1-(fluoromethyl)ethyl]oxy}-5-[3-(3-glycyl-2,3,4,5-tetrahydro-1H-3-benzazepin-7-yl)-1,2,4-oxadiazol-5-yl]benzonitrile
5-{3-[3-(2,3-dihydroxypropyl)-2,3,4,5-tetrahydro-1H-3-benzazepin-7-yl]-1,2,4-oxadiazol-5-yl}-2-(3-fluoro-1-pyrrolidinyl)benzonitrile
5-[3-(2-D-allothreonyl-5-methyl-1,2,3,4-tetrahydro-6-isoquinolinyl)-1,2,4-oxadiazol-5-yl]-2-[(1-methylethyl)oxy]benzonitrile
2-(3-fluoro-1-pyrrolidinyl)-5-[3-(3-glycyl-2,3,4,5-tetrahydro-1H-3-benzazepin-7-yl)-1,2,4-oxadiazol-5-yl]benzonitrile
5-[3-(3-D-allothreonyl-2,3,4,5-tetrahydro-1H-3-benzazepin-7-yl)-1,2,4-oxadiazol-5-yl]-2-[(1-methylethyl)oxy]benzonitrile
2-[(1-methylethyl)oxy]-5-[3-(3-L-threonyl-2,3,4,5-tetrahydro-1H-3-benzazepin-7-yl)-1,2,4-oxadiazol-5-yl]benzonitrile
5-{3-[3-(2,3-dihydroxypropyl)-2,3,4,5-tetrahydro-1H-3-benzazepin-7-yl]-1,2,4-oxadiazol-5-yl}-2-{[2-fluoro-1-(fluoromethyl)ethyl]oxy}benzonitrile
2-(ethyloxy)-5-[3-(3-glycyl-2,3,4,5-tetrahydro-1H-3-benzazepin-7-yl)-1,2,4-oxadiazol-5-yl]benzonitrile
5-[3-(3-glycyl-2,3,4,5-tetrahydro-1H-3-benzazepin-7-yl)-1,2,4-oxadiazol-5-yl]-2-(propyloxy)benzonitrile
5-{3-[3-(4-aminobutanoyl)-2,3,4,5-tetrahydro-1H-3-benzazepin-7-yl]-1,2,4-oxadiazol-5-yl}-2-[(1-methylethyl)oxy]benzonitrile
5-(3-{2-[2-hydroxy-1-(hydroxymethyl)ethyl]-5-methyl-1,2,3,4-tetrahydro-6-isoquinolinyl}-1,2,4-oxadiazol-5-yl)-2-[(1-methylethyl)oxy]benzonitrile
5-{3-[3-(N-methyl-b-alanyl)-2,3,4,5-tetrahydro-1H-3-benzazepin-7-yl]-1,2,4-oxadiazol-5-yl}-2-[(1-methylethyl)oxy]benzonitrile
5-(3-{3-[2-hydroxy-1-(hydroxymethyl)ethyl]-2,3,4,5-tetrahydro-1H-3-benzazepin-7-yl}-1,2,4-oxadiazol-5-yl)-2-[(1-methylethyl)oxy]benzonitrile
5-[3-(3-β-alanyl-2,3,4,5-tetrahydro-1H-3-benzazepin-7-yl)-1,2,4-oxadiazol-5-yl]-2-[(1-methylethyl)oxy]benzonitrile
5-{3-[3-(2,3-dihydroxypropyl)-2,3,4,5-tetrahydro-1H-3-benzazepin-7-yl]-1,2,4-oxadiazol-5-yl}-2-(ethyloxy)benzonitrile
5-(3-{2-[(2S)-2,3-dihydroxypropyl]-5-methyl-1,2,3,4-tetrahydro-6-isoquinolinyl}-1,2,4-oxadiazol-5-yl)-2-[(1-methylethyl)oxy]benzonitrile
5-(3-{3-[(2S)-2,3-dihydroxypropyl]-2,3,4,5-tetrahydro-1H-3-benzazepin-7-yl}-1,2,4-oxadiazol-5-yl)-2-[(1-methylethyl)oxy]benzonitrile
5-(3-{2-[(2R)-2,3-dihydroxypropyl]-5-methyl-1,2,3,4-tetrahydro-6-isoquinolinyl}-1,2,4-oxadiazol-5-yl)-2-[(1-methylethyl)oxy]benzonitrile
5-(3-{3-[(2R)-2,3-dihydroxypropyl]-2,3,4,5-tetrahydro-1H-3-benzazepin-7-yl}-1,2,4-oxadiazol-5-yl)-2-[(1-methylethyl)oxy]benzonitrile
2-[7-(5-{3-cyano-4-[(1-methylethyl)oxy]phenyl}-1,2,4-oxadiazol-3-yl)-1,2,4,5-tetrahydro-3H-3-benzazepin-3-yl]-N-[(2S)-2-hydroxypropyl]acetamide
5-{3-[2-(2,3-dihydroxypropyl)-1,2,3,4-tetrahydro-5-isoquinolinyl]-1,2,4-oxadiazol-5-yl}-2-[(1-methylethyl)oxy]benzonitrile
2-[(1-methylethyl)oxy]-5-[3-(3-L-threonyl-2,3,4,5-tetrahydro-1H-3-benzazepin-7-yl)-1,2,4-oxadiazol-5-yl]-3-pyridinecarbonitrile
2-[7-(5-{3-cyano-4-[(1-methylethyl)oxy]phenyl}-1,2,4-oxadiazol-3-yl)-1,2,4,5-tetrahydro-3H-3-benzazepin-3-yl]-N-[(1R)-2-hydroxy-1-methylethyl]acetamide
2-[(1-methylethyl)oxy]-5-[3-(5-methyl-2-L-threonyl-1,2,3,4-tetrahydro-6-isoquinolinyl)-1,2,4-oxadiazol-5-yl]-3-pyridinecarbonitrile
2-[7-(5-{3-cyano-4-[(1-methylethyl)oxy]phenyl}-1,2,4-oxadiazol-3-yl)-1,2,4,5-tetrahydro-3H-3-benzazepin-3-yl]-N-[(1S)-2-hydroxy-1-methylethyl]acetamide
2-[(1-methylethyl)oxy]-5-{3-[3-(2-methyl-L-seryl)-2,3,4,5-tetrahydro-1H-3-benzazepin-7-yl]-1,2,4-oxadiazol-5-yl}benzonitrile
2-[(1-methylethyl)oxy]-5-{3-[5-methyl-2-(2-methyl-L-seryl)-1,2,3,4-tetrahydro-6-isoquinolinyl]-1,2,4-oxadiazol-5-yl}benzonitrile
5-(3-{3-[4-(methylamino)butanoyl]-2,3,4,5-tetrahydro-1H-3-benzazepin-7-yl}-1,2,4-oxadiazol-5-yl)-2-[(1-methylethyl)oxy]benzonitrile
2-[(1-methylethyl)oxy]-5-{3-[3-(2-methyl-D-seryl)-2,3,4,5-tetrahydro-1H-3-benzazepin-7-yl]-1,2,4-oxadiazol-5-yl}benzonitrile
2-[(1-methylethyl)oxy]-5-{3-[5-methyl-2-(2-methyl-D-seryl)-1,2,3,4-tetrahydro-6-isoquinolinyl]-1,2,4-oxadiazol-5-yl}benzonitrile
5-(3-{3-[N-(2-hydroxyethyl)glycyl]-2,3,4,5-tetrahydro-1H-3-benzazepin-7-yl}-1,2,4-oxadiazol-5-yl)-2-[(1-methylethyl)oxy]benzonitrile
5-[3-(2-glycyl-1,2,3,4-tetrahydro-5-isoquinolinyl)-1,2,4-oxadiazol-5-yl]-2-[(1-methylethyl)oxy]benzonitrile
5-[3-(2-β-alanyl-1,2,3,4-tetrahydro-5-isoquinolinyl)-1,2,4-oxadiazol-5-yl]-2-[(1-methylethyl)oxy]benzonitrile
2-[(1-methylethyl)oxy]-5-[3-(2-L-seryl-1,2,3,4-tetrahydro-5-isoquinolinyl)-1,2,4-oxadiazol-5-yl]benzonitrile
2-[(1-methylethyl)oxy]-5-[3-(2-D-seryl-1,2,3,4-tetrahydro-5-isoquinolinyl)-1,2,4-oxadiazol-5-yl]benzonitrile
2-[(1-methylethyl)oxy]-5-{3-[3-(4-morpholinylacetyl)-2,3,4,5-tetrahydro-1H-3-benzazepin-7-yl]-1,2,4-oxadiazol-5-yl}benzonitrile
2-[(1-methylethyl)oxy]-5-(3-{3-[2-(4-morpholinyl)-2-oxoethyl]-2,3,4,5-tetrahydro-1H-3-benzazepin-7-yl}-1,2,4-oxadiazol-5-yl)benzonitrile
5-(3-{3-[2-(3-hydroxy-1-azetidinyl)-2-oxoethyl]-2,3,4,5-tetrahydro-1H-3-benzazepin-7-yl}-1,2,4-oxadiazol-5-yl)-2-[(1-methylethyl)oxy]benzonitrile
2-(3-fluoro-1-azetidinyl)-5-[3-(3-glycyl-2,3,4,5-tetrahydro-1H-3-benzazepin-7-yl)-1,2,4-oxadiazol-5-yl]benzonitrile
5-(3-{3-[2-hydroxy-1-(hydroxymethyl)ethyl]-2,3,4,5-tetrahydro-1H-3-benzazepin-7-yl}-1,2,4-oxadiazol-5-yl)-2-[(1-methylethyl)oxy]-3-pyridinecarbonitrile
5-(3-{3-[(2S)-2,3-dihydroxypropyl]-2,3,4,5-tetrahydro-1H-3-benzazepin-7-yl}-1,2,4-oxadiazol-5-yl)-2-[(1-methylethyl)oxy]-3-pyridinecarbonitrile
5-(3-{3-[(2R)-2,3-dihydroxypropyl]-2,3,4,5-tetrahydro-1H-3-benzazepin-7-yl}-1,2,4-oxadiazol-5-yl)-2-[(1-methylethyl)oxy]-3-pyridinecarbonitrile
5-[3-(3-{N-(1R)-2-hydroxy-1-methylethyl]glycyl}-2,3,4,5-tetrahydro-1H-3-benzazepin-7-yl)-1,2,4-oxadiazol-5-yl]-2-[(1-methylethyl)oxy]benzonitrile
5-[3-(3-{N-[(1S)-2-hydroxy-1-methylethyl]glycyl}-2,3,4,5-tetrahydro-1H-3-benzazepin-7-yl)-1,2,4-oxadiazol-5-yl]-2-[(1-methylethyl)oxy]benzonitrile
methyl {2-[7-(5-{3-cyano-4-[(1-methylethyl)oxy]phenyl}-1,2,4-oxadiazol-3-yl)-1,2,4,5-tetrahydro-3H-3-benzazepin-3-yl]ethyl}carbamate
N-{2-[7-(5-{3-cyano-4-[(1-methylethyl)oxy]phenyl}-1,2,4-oxadiazol-3-yl)-1,2,4,5-tetrahydro-3H-3-benzazepin-3-yl]ethyl}acetamide
methyl {3-[7-(5-{3-cyano-4-[(1-methylethyl)oxy]phenyl}-1,2,4-oxadiazol-3-yl)-1,2,4,5-tetrahydro-3H-3-benzazepin-3-yl]propyl}carbamate
N-{3-[7-(5-{3-cyano-4-[(1-methylethyl)oxy]phenyl}-1,2,4-oxadiazol-3-yl)-1,2,4,5-tetrahydro-3H-3-benzazepin-3-yl]propyl}propanamide
5-{3-[3-(2,3-dihydroxypropyl)-2,3,4,5-tetrahydro-1H-3-benzazepin-7-yl]-1,2,4-oxadiazol-5-yl}-2-(3-fluoro-1-azetidinyl)benzonitrile
N-{2-[7-(5-{3-cyano-4-[(1-methylethyl)oxy]phenyl}-1,2,4-oxadiazol-3-yl)-1,2,4,5-tetrahydro-3H-3-benzazepin-3-yl]ethyl}-N′-ethylurea
5-(3-{3-[(3-hydroxy-1-azetidinyl)acetyl]-2,3,4,5-tetrahydro-1H-3-benzazepin-7-yl}-1,2,4-oxadiazol-5-yl)-2-[(1-methylethyl)oxy]benzonitrile
5-[3-(3-glycyl-2,3,4,5-tetrahydro-1H-3-benzazepin-7-yl)-1,2,4-oxadiazol-5-yl]-2-(propyloxy)-3-pyridinecarbonitrile
5-(3-{3-[2-hydroxy-1-(hydroxymethyl)ethyl]-2,3,4,5-tetrahydro-1H-3-benzazepin-7-yl}-1,2,4-oxadiazol-5-yl)-2-[(1-methylethyl)amino]-3-pyridinecarbonitrile
5-(3-{3-[2-hydroxy-1-(hydroxymethyl)ethyl]-2,3,4,5-tetrahydro-1H-3-benzazepin-7-yl}-1,2,4-oxadiazol-5-yl)-2-(propyloxy)-3-pyridinecarbonitrile
2-(ethyloxy)-5-(3-{3-[2-hydroxy-1-(hydroxymethyl)ethyl]-2,3,4,5-tetrahydro-1H-3-benzazepin-7-yl}-1,2,4-oxadiazol-5-yl)-3-pyridinecarbonitrile
2-[7-(5-{3-cyano-4-[(1-methylethyl)oxy]phenyl}-1,2,4-oxadiazol-3-yl)-1,2,4,5-tetrahydro-3H-3-benzazepin-3-yl]-N-(2-hydroxyethyl)acetamide
5-[3-(3-glycyl-2,3,4,5-tetrahydro-1H-3-benzazepin-7-yl)-1,2,4-oxadiazol-5-yl]-2-[(1-methylethyl)amino]-3-pyridinecarbonitrile
2-(ethyloxy)-5-[3-(3-glycyl-2,3,4,5-tetrahydro-1H-3-benzazepin-7-yl)-1,2,4-oxadiazol-5-yl]-3-pyridinecarbonitrile
5-(3-{3-[(2S)-2,3-dihydroxypropyl]-2,3,4,5-tetrahydro-1H-3-benzazepin-7-yl}-1,2,4-oxadiazol-5-yl)-2-[(1-methylethyl)amino]-3-pyridinecarbonitrile
5-(3-{3-[(2S)-2,3-dihydroxypropyl]-2,3,4,5-tetrahydro-1H-3-benzazepin-7-yl}-1,2,4-oxadiazol-5-yl)-2-(propyloxy)-3-pyridinecarbonitrile
5-(3-{3-[(2S)-2,3-dihydroxypropyl]-2,3,4,5-tetrahydro-1H-3-benzazepin-7-yl}-1,2,4-oxadiazol-5-yl)-2-(ethyloxy)-3-pyridinecarbonitrile
5-{3-[3-(2-methylalanyl)-2,3,4,5-tetrahydro-1H-3-benzazepin-7-yl]-1,2,4-oxadiazol-5-yl}-2-[(1-methylethyl)oxy]benzonitrile
2-[6-(5-{3-cyano-4-[(1-methylethyl)oxy]phenyl}-1,2,4-oxadiazol-3-yl)-5-methyl-3,4-dihydro-2(1H)-isoquinolinyl]-N-(1R)-2-hydroxy-1-methylethyl]acetamide
2-[6-(5-{3-cyano-4-[(1-methylethyl)oxy]phenyl}-1,2,4-oxadiazol-3-yl)-5-methyl-3,4-dihydro-2(1H)-isoquinolinyl]-N-[(1S)-2-hydroxy-1-methylethyl]acetamide
2-[6-(5-{3-cyano-4-[(1-methylethyl)oxy]phenyl}-1,2,4-oxadiazol-3-yl)-5-methyl-3,4-dihydro-2(1H)-isoquinolinyl]-N-[(2R)-2-hydroxypropyl]acetamide
2-[6-(5-{3-cyano-4-[(1-methylethyl)oxy]phenyl}-1,2,4-oxadiazol-3-yl)-5-methyl-3,4-dihydro-2(1H)-isoquinolinyl]-N-[(2S)-2-hydroxypropyl]acetamide
2-[6-(5-{3-cyano-4-[(1-methylethyl)oxy]phenyl}-1,2,4-oxadiazol-3-yl)-5-methyl-3,4-dihydro-2(1H)-isoquinolinyl]-N-(2-hydroxyethyl)acetamide
5-(3-{2-[2-(3-hydroxy-1-azetidinyl)-2-oxoethyl]-5-methyl-1,2,3,4-tetrahydro-6-isoquinolinyl}-1,2,4-oxadiazol-5-yl)-2-[(1-methylethyl)oxy]benzonitrile
2-[6-(5-{3-cyano-4-[(1-methylethyl)oxy]phenyl}-1,2,4-oxadiazol-3-yl)-5-methyl-3,4-dihydro-2(1H)-isoquinolinyl]-N-[(2S)-2,3-dihydroxypropyl]acetamide
2-[6-(5-{3-cyano-4-[(1-methylethyl)oxy]phenyl}-1,2,4-oxadiazol-3-yl)-5-methyl-3,4-dihydro-2(1H)-isoquinolinyl]-N-[2-hydroxy-1-(hydroxymethyl)ethyl]acetamide
5-[3-(2-{N-[(2S)-2,3-dihydroxypropyl]glycyl}-5-methyl-1,2,3,4-tetrahydro-6-isoquinolinyl)-1,2,4-oxadiazol-5-yl]-2-[(1-methylethyl)oxy]benzonitrile
5-[3-(2-{N-[2-hydroxy-1-(hydroxymethyl)ethyl]glycyl}-5-methyl-1,2,3,4-tetrahydro-6-isoquinolinyl)-1,2,4-oxadiazol-5-yl]-2-[(1-methylethyl)oxy]benzonitrile
5-[3-(2-{N-(1R)-2-hydroxy-1-methylethyl]glycyl}-5-methyl-1,2,3,4-tetrahydro-6-isoquinolinyl)-1,2,4-oxadiazol-5-yl]-2-[(1-methylethyl)oxy]benzonitrile
5-[3-(2-{N-[(2R)-2-hydroxypropyl]glycyl}-5-methyl-1,2,3,4-tetrahydro-6-isoquinolinyl)-1,2,4-oxadiazol-5-yl]-2-[(1-methylethyl)oxy]benzonitrile
5-[3-(2-{N-[(1S)-2-hydroxy-1-methylethyl]glycyl}-5-methyl-1,2,3,4-tetrahydro-6-isoquinolinyl)-1,2,4-oxadiazol-5-yl]-2-[(1-methylethyl)oxy]benzonitrile
5-(3-{2-[N-(2-hydroxyethyl)glycyl]-5-methyl-1,2,3,4-tetrahydro-6-isoquinolinyl}-1,2,4-oxadiazol-5-yl)-2-[(1-methylethyl)oxy]benzonitrile
5-(3-{2-[(2S)-2,3-dihydroxypropyl]-5-methyl-1,2,3,4-tetrahydro-6-isoquinolinyl}-1,2,4-oxadiazol-5-yl)-2-[(1-methylethyl)oxy]-3-pyridinecarbonitrile
5-(3-{2-[(2R)-2,3-dihydroxypropyl]-5-methyl-1,2,3,4-tetrahydro-6-isoquinolinyl}-1,2,4-oxadiazol-5-yl)-2-[(1-methylethyl)oxy]-3-pyridinecarbonitrile
5-(3-{2-[2-hydroxy-1-(hydroxymethyl)ethyl]-5-methyl-1,2,3,4-tetrahydro-6-isoquinolinyl}-1,2,4-oxadiazol-5-yl)-2-[(1-methylethyl)oxy]-3-pyridinecarbonitrile
5-(3-{3-[(2S)-2,3-dihydroxypropyl]-2,3,4,5-tetrahydro-1H-3-benzazepin-7-yl}-1,2,4-oxadiazol-5-yl)-2-[(2,2,2-trifluoroethyl)oxy]benzonitrile
2-[6-(5-{3-chloro-4-[(1-methylethyl)oxy]phenyl}-1,2,4-oxadiazol-3-yl)-5-methyl-3,4-dihydro-2(1H)-isoquinolinyl]-N-(2-hydroxyethyl)acetamide
5-[3-(2-glycyl-5-methyl-1,2,3,4-tetrahydro-6-isoquinolinyl)-1,2,4-oxadiazol-5-yl]-2-[(1-methylethyl)oxy]-3-pyridinecarbonitrile
5-(3-{3-[(2S)-2,3-dihydroxypropyl]-8-methyl-2,3,4,5-tetrahydro-1H-3-benzazepin-7-yl}-1,2,4-oxadiazol-5-yl)-2-[(1-methylethyl)oxy]benzonitrile
2-({2-[6-(5-{3-chloro-4-[(1-methylethyl)oxy]phenyl}-1,2,4-oxadiazol-3-yl)-5-methyl-3,4-dihydro-2(1H)-isoquinolinyl]-2-oxoethyl}amino)ethanol
5-[3-(3-glycyl-6-methyl-2,3,4,5-tetrahydro-1H-3-benzazepin-7-yl)-1,2,4-oxadiazol-5-yl]-2-[(1-methylethyl)oxy]benzonitrile
{2-[6-(5-{5-chloro-6-[(1-methylethyl)oxy]-3-pyridinyl}-1,2,4-oxadiazol-3-yl)-5-methyl-3,4-dihydro-2(1H)-isoquinolinyl]-2-oxoethyl}amine
5-(3-{3-[(2S)-2,3-dihydroxypropyl]-2,3,4,5-tetrahydro-1H-3-benzazepin-7-yl}-1,2,4-oxadiazol-5-yl)-2-(propylamino)-3-pyridinecarbonitrile
5-(3-{3-[(2S)-2,3-dihydroxypropyl]-2,3,4,5-tetrahydro-1H-3-benzazepin-7-yl}-1,2,4-oxadiazol-5-yl)-2-(propylamino)-3-pyridinecarbonitrile
2-[6-(5-{5-chloro-6-[(1-methylethyl)oxy]-3-pyridinyl}-1,2,4-oxadiazol-3-yl)-5-methyl-3,4-dihydro-2(1H)-isoquinolinyl]-1,3-propanediol
5-(3-{3-[(2S)-2,3-dihydroxypropyl]-6-methyl-2,3,4,5-tetrahydro-1H-3-benzazepin-7-yl}-1,2,4-oxadiazol-5-yl)-2-(propylamino)-3-pyridinecarbonitrile
2-[7-(5-{3-cyano-4-[(1-methylethyl)oxy]phenyl}-1,2,4-oxadiazol-3-yl)-6-methyl-1,2,4,5-tetrahydro-3H-3-benzazepin-3-yl]-N-(2-hydroxyethyl)acetamide
2-[7-(5-{3-cyano-4-[(1-methylethyl)oxy]phenyl}-1,2,4-oxadiazol-3-yl)-6-methyl-1,2,4,5-tetrahydro-3H-3-benzazepin-3-yl]-N-[(2R)-2-hydroxypropyl]acetamide
2-[7-(5-{3-cyano-4-[(1-methylethyl)oxy]phenyl}-1,2,4-oxadiazol-3-yl)-6-methyl-1,2,4,5-tetrahydro-3H-3-benzazepin-3-yl]-N-[(2S)-2-hydroxypropyl]acetamide
5-(3-{3-[2-hydroxy-1-(hydroxymethyl)ethyl]-6-methyl-2,3,4,5-tetrahydro-1H-3-benzazepin-7-yl}-1,2,4-oxadiazol-5-yl)-2-(propylamino)-3-pyridinecarbonitrile
5-(3-{3-[2-hydroxy-1-(hydroxymethyl)ethyl]-2,3,4,5-tetrahydro-1H-3-benzazepin-7-yl}-1,2,4-oxadiazol-5-yl)-2-(propylamino)-3-pyridinecarbonitrile
5-[3-(3-glycyl-2,3,4,5-tetrahydro-1H-3-benzazepin-7-yl)-1,2,4-oxadiazol-5-yl]-2-(propylamino)-3-pyridinecarbonitrile
5-[3-(3-glycyl-2,3,4,5-tetrahydro-1H-3-benzazepin-7-yl)-1,2,4-oxadiazol-5-yl]-2-(propylamino)-3-pyridinecarbonitrile
5-(3-{3-[(2S)-2,3-dihydroxypropyl]-6-methyl-2,3,4,5-tetrahydro-1H-3-benzazepin-7-yl}-1,2,4-oxadiazol-5-yl)-2-[(1-methylethyl)oxy]benzonitrile
5-(3-{3-[(2R)-2,3-dihydroxypropyl]-6-methyl-2,3,4,5-tetrahydro-1H-3-benzazepin-7-yl}-1,2,4-oxadiazol-5-yl)-2-[(1-methylethyl)oxy]benzonitrile
5-(3-{3-[N-(2-hydroxyethyl)glycyl]-6-methyl-2,3,4,5-tetrahydro-1H-3-benzazepin-7-yl}-1,2,4-oxadiazol-5-yl)-2-[(1-methylethyl)oxy]benzonitrile
5-(3-{3-[2-hydroxy-1-(hydroxymethyl)ethyl]-6-methyl-2,3,4,5-tetrahydro-1H-3-benzazepin-7-yl}-1,2,4-oxadiazol-5-yl)-2-[(1-methylethyl)oxy]benzonitrile
2-[7-(5-{3-cyano-4-[(1-methylethyl)oxy]phenyl}-1,2,4-oxadiazol-3-yl)-1,2,4,5-tetrahydro-3H-3-benzazepin-3-yl]-N-(3-hydroxypropyl)acetamide
2-[7-(5-{3-cyano-4-[(1-methylethyl)oxy]phenyl}-1,2,4-oxadiazol-3-yl)-6-methyl-1,2,4,5-tetrahydro-3H-3-benzazepin-3-yl]-N-[(1S)-2-hydroxy-1-methylethyl]acetamide
2-[7-(5-{3-cyano-4-[(1-methylethyl)oxy]phenyl}-1,2,4-oxadiazol-3-yl)-6-methyl-1,2,4,5-tetrahydro-3H-3-benzazepin-3-yl]-N-(1R)-2-hydroxy-1-methylethyl]acetamide
2-[7-(5-{3-cyano-4-[(1-methylethyl)oxy]phenyl}-1,2,4-oxadiazol-3-yl)-1,2,4,5-tetrahydro-3H-3-benzazepin-3-yl]-N-(2-hydroxyethyl)-2-methylpropanamide
2-[7-(5-{3-cyano-4-[(1-methylethyl)oxy]phenyl}-1,2,4-oxadiazol-3-yl)-1,2,4,5-tetrahydro-3H-3-benzazepin-3-yl]-N-(2-hydroxyethyl)propanamide
2-[7-(5-{3-cyano-4-[(1-methylethyl)oxy]phenyl}-1,2,4-oxadiazol-3-yl)-1,2,4,5-tetrahydro-3H-3-benzazepin-3-yl]-N-(1R)-2-hydroxy-1-methylethyl]propanamide
2-[7-(5-{3-cyano-4-[(1-methylethyl)oxy]phenyl}-1,2,4-oxadiazol-3-yl)-1,2,4,5-tetrahydro-3H-3-benzazepin-3-yl]-N-(1R)-2-hydroxy-1-methylethyl]-2-methylpropanamide
(2R)-3-[7-(5-{3-cyano-4-[(1-methylethyl)oxy]phenyl}-1,2,4-oxadiazol-3-yl)-1,2,4,5-tetrahydro-3H-3-benzazepin-3-yl]-2-hydroxy-N-(2-hydroxyethyl)propanamide
(2R)-3-[7-(5-{3-cyano-4-[(1-methylethyl)oxy]phenyl}-1,2,4-oxadiazol-3-yl)-1,2,4,5-tetrahydro-3H-3-benzazepin-3-yl]-2-hydroxy-N-(1R)-2-hydroxy-1-methylethyl]propanamide
5-(3-{2-[(2R)-2,3-dihydroxypropyl]-1,2,3,4-tetrahydro-6-isoquinolinyl}-1,2,4-oxadiazol-5-yl)-2-[(1-methylethyl)oxy]-3-pyridinecarbonitrile
5-(3-{2-[(2S)-2,3-dihydroxypropyl]-1,2,3,4-tetrahydro-6-isoquinolinyl}-1,2,4-oxadiazol-5-yl)-2-[(1-methylethyl)oxy]-3-pyridinecarbonitrile
5-(3-{2-[2-hydroxy-1-(hydroxymethyl)ethyl]-1,2,3,4-tetrahydro-6-isoquinolinyl}-1,2,4-oxadiazol-5-yl)-2-[(1-methylethyl)oxy]-3-pyridinecarbonitrile
2-[6-(5-{3-cyano-4-[(1-methylethyl)oxy]phenyl}-1,2,4-oxadiazol-3-yl)-3,4-dihydro-2(1H)-isoquinolinyl]-N-[(1S)-2-hydroxy-1-methylethyl]acetamide
2-[6-(5-{5-cyano-6-[(1-methylethyl)oxy]-3-pyridinyl}-1,2,4-oxadiazol-3-yl)-5-methyl-3,4-dihydro-2(1H)-isoquinolinyl]-N-[(1S)-2-hydroxy-1-methylethyl]acetamide
5-(3-{3-[(2R)-2,3-dihydroxypropyl]-2,3,4,5-tetrahydro-1H-3-benzazepin-7-yl}-1,2,4-oxadiazol-5-yl)-2-[(1-methylethyl)amino]-3-pyridinecarbonitrile
5-(3-{3-[(2R)-2,3-dihydroxypropyl]-6-methyl-2,3,4,5-tetrahydro-1H-3-benzazepin-7-yl}-1,2,4-oxadiazol-5-yl)-2-[(1-methylethyl)amino]-3-pyridinecarbonitrile
5-(3-{3-[(2S)-2,3-dihydroxypropyl]-6-methyl-2,3,4,5-tetrahydro-1H-3-benzazepin-7-yl}-1,2,4-oxadiazol-5-yl)-2-[(1-methylethyl)amino]-3-pyridinecarbonitrile
5-(3-{2-[(2R)-2,3-dihydroxypropyl]-5-methyl-1,2,3,4-tetrahydro-6-isoquinolinyl}-1,2,4-oxadiazol-5-yl)-2-[(1-methylethyl)amino]-3-pyridinecarbonitrile
5-(3-{3-[2-hydroxy-1-(hydroxymethyl)ethyl]-6-methyl-2,3,4,5-tetrahydro-1H-3-benzazepin-7-yl}-1,2,4-oxadiazol-5-yl)-2-[(1-methylethyl)amino]-3-pyridinecarbonitrile
5-(3-{2-[2-hydroxy-1-(hydroxymethyl)ethyl]-5-methyl-1,2,3,4-tetrahydro-6-isoquinolinyl}-1,2,4-oxadiazol-5-yl)-2-[(1-methylethyl)amino]-3-pyridinecarbonitrile
5-(3-{2-[(2S)-2,3-dihydroxypropyl]-5-methyl-1,2,3,4-tetrahydro-6-isoquinolinyl}-1,2,4-oxadiazol-5-yl)-2-[(1-methylethyl)amino]-3-pyridinecarbonitrile
5-(3-{2-[(2R)-2,3-dihydroxypropyl]-1,2,3,4-tetrahydro-5-isoquinolinyl}-1,2,4-oxadiazol-5-yl)-2-[(1-methylethyl)oxy]benzonitrile
5-(3-{2-[(2R)-2,3-dihydroxypropyl]-1,2,3,4-tetrahydro-5-isoquinolinyl}-1,2,4-oxadiazol-5-yl)-2-[(1-methylethyl)oxy]benzonitrile
5-(3-{2-[2-hydroxy-1-(hydroxymethyl)ethyl]-1,2,3,4-tetrahydro-5-isoquinolinyl}-1,2,4-oxadiazol-5-yl)-2-[(1-methylethyl)oxy]benzonitrile
5-(5-{3-[(2R)-2,3-dihydroxypropyl]-2,3,4,5-tetrahydro-1H-3-benzazepin-7-yl}-1,3,4-thiadiazol-2-yl)-2-[(1-methylethyl)oxy]benzonitrile
5-(5-{3-[(2S)-2,3-dihydroxypropyl]-2,3,4,5-tetrahydro-1H-3-benzazepin-7-yl}-1,3,4-thiadiazol-2-yl)-2-[(1-methylethyl)oxy]benzonitrile
2-[7-(5-{3-cyano-4-[(1-methylethyl)oxy]phenyl}-1,2,4-oxadiazol-3-yl)-3,4-dihydro-2(1H)-isoquinolinyl]-N-[(1S)-2-hydroxy-1-methylethyl]acetamide
5-(5-{2-[2-hydroxy-1-(hydroxymethyl)ethyl]-5-methyl-1,2,3,4-tetrahydro-6-isoquinolinyl}-1,3,4-thiadiazol-2-yl)-2-[(1-methylethyl)oxy]benzonitrile
2-[7-(5-{3-cyano-4-[(1-methylethyl)oxy]phenyl}-1,2,4-oxadiazol-3-yl)-1,2,4,5-tetrahydro-3H-3-benzazepin-3-yl]-N-(3,3,3-trifluoro-2-hydroxypropyl)acetamide
2-[7-(5-{3-cyano-4-[(1-methylethyl)oxy]phenyl}-1,2,4-oxadiazol-3-yl)-6-methyl-1,2,4,5-tetrahydro-3H-3-benzazepin-3-yl]-N-[2-hydroxy-1-(hydroxymethyl)ethyl]acetamide
5-(3-{3-[N-(2,3-dihydroxypropyl)glycyl]-6-methyl-2,3,4,5-tetrahydro-1H-3-benzazepin-7-yl}-1,2,4-oxadiazol-5-yl)-2-[(1-methylethyl)oxy]benzonitrile
5-(3-{3-[N-(2,3-dihydroxypropyl)glycyl]-8-methyl-2,3,4,5-tetrahydro-1H-3-benzazepin-7-yl}-1,2,4-oxadiazol-5-yl)-2-[(1-methylethyl)oxy]benzonitrile
5-[3-(3-{N-[2-hydroxy-1-(hydroxymethyl)ethyl]glycyl}-6-methyl-2,3,4,5-tetrahydro-1H-3-benzazepin-7-yl)-1,2,4-oxadiazol-5-yl]-2-[(1-methylethyl)oxy]benzonitrile
2-({2-[6-(5-{3-chloro-4-[(1-methylethyl)oxy]phenyl}-1,2,4-oxadiazol-3-yl)-5-methyl-3,4-dihydro-2(1H)-isoquinolinyl]-2-oxoethyl}amino)-1,3-propanediol
5-[3-(3-glycyl-2,3,4,5-tetrahydro-1H-3-benzazepin-7-yl)-1,2,4-oxadiazol-5-yl]-2-[(2,2,2-trifluoroethyl)oxy]benzonitrile
2-[6-(5-{3-chloro-4-[(1-methylethyl)oxy]phenyl}-1,2,4-oxadiazol-3-yl)-5-methyl-3,4-dihydro-2(1H)-isoquinolinyl]-N-[(2S)-2,3-dihydroxypropyl]acetamide
2-[6-(5-{3-chloro-4-[(1-methylethyl)oxy]phenyl}-1,2,4-oxadiazol-3-yl)-5-methyl-3,4-dihydro-2(1H)-isoquinolinyl]-N-[2-hydroxy-1-(hydroxymethyl)ethyl]acetamide
2-[7-(5-{3-cyano-4-[(1-methylethyl)oxy]phenyl}-1,2,4-oxadiazol-3-yl)-1,2,4,5-tetrahydro-3H-3-benzazepin-3-yl]-N-[(2S)-2,3-dihydroxypropyl]acetamide
2-[7-(5-{3-cyano-4-[(1-methylethyl)oxy]phenyl}-1,2,4-oxadiazol-3-yl)-1,2,4,5-tetrahydro-3H-3-benzazepin-3-yl]-N-[2-hydroxy-1-(hydroxymethyl)ethyl]acetamide
5-[3-(3-{N-[(2S)-2,3-dihydroxypropyl]glycyl}-2,3,4,5-tetrahydro-1H-3-benzazepin-7-yl)-1,2,4-oxadiazol-5-yl]-2-[(1-methylethyl)oxy]benzonitrile hydrochloride
5-[3-(3-{N-[2-hydroxy-1-(hydroxymethyl)ethyl]glycyl}-2,3,4,5-tetrahydro-1H-3-benzazepin-7-yl)-1,2,4-oxadiazol-5-yl]-2-[(1-methylethyl)oxy]benzonitrile hydrochloride
5-[3-(2-{N-[(2S)-2-hydroxypropyl]glycyl}-5-methyl-1,2,3,4-tetrahydro-6-isoquinolinyl)-1,2,4-oxadiazol-5-yl]-2-[(1-methylethyl)oxy]benzonitrile hydrochloride
5-(5-{3-[2-hydroxy-1-(hydroxymethyl)ethyl]-2,3,4,5-tetrahydro-1H-3-benzazepin-7-yl}-1,3,4-thiadiazol-2-yl)-2-[(1-methylethyl)oxy]benzonitrile
5-(5-{3-[N-(2-hydroxyethyl)glycyl]-2,3,4,5-tetrahydro-1H-3-benzazepin-7-yl}-1,3,4-thiadiazol-2-yl)-2-[(1-methylethyl)oxy]benzonitrile
5-(5-{2-[N-(2-hydroxyethyl)glycyl]-5-methyl-1,2,3,4-tetrahydro-6-isoquinolinyl}-1,3,4-thiadiazol-2-yl)-2-[(1-methylethyl)oxy]benzonitrile
2-({2-[6-(5-{3-chloro-4-[(1-methylethyl)oxy]phenyl}-1,3,4-thiadiazol-2-yl)-5-methyl-3,4-dihydro-2(1H)-isoquinolinyl]-2-oxoethyl}amino)ethanol
2-[6-(5-{3-chloro-4-[(1-methylethyl)oxy]phenyl}-1,3,4-thiadiazol-2-yl)-5-methyl-3,4-dihydro-2(1H)-isoquinolinyl]-1,3-propanediol
   or pharmaceutically acceptable salts thereof.

Suitably a compound of formula (I) is
5-(3-{2-[2-hydroxy-1-(hydroxymethyl)ethyl]-5-methyl-1,2,3,4-tetrahydro-6-isoquinolinyl}-1,2,4-oxadiazol-5-yl)-2-[(1-methylethyl)oxy]benzonitrile
   or a pharmaceutically acceptable salt thereof.

Suitably a compound of formula (I) is
5-(3-{2-[2-hydroxy-1-(hydroxymethyl)ethyl]-5-methyl-1,2,3,4-tetrahydro-6-isoquinolinyl}-1,2,4-oxadiazol-5-yl)-2-[(1-methylethyl)oxy]-3-pyridinecarbonitrile
   or a pharmaceutically acceptable salt thereof.

Suitably a compound of formula (I) is 5-(5-{2-[2-hydroxy-1-(hydroxymethyl)ethyl]-5-methyl-1,2,3,4-tetrahydro-6-isoquinolinyl}-1,3,4-thiadiazol-2-yl)-2-[(1-methylethyl)oxy]benzonitrile or a pharmaceutically acceptable salt thereof.

Pharmaceutically acceptable derivatives of compounds of formula (I) include any pharmaceutically acceptable salt, ester or salt of such ester of a compound of formula (I) which, upon administration to the recipient is capable of providing (directly or indirectly) a compound of formula (I) or an active metabolite or residue thereof.

The compounds of formula (I) can form salts. It will be appreciated that for use in medicine the salts of the compounds of formula (I) should be pharmaceutically acceptable. Suitable pharmaceutically acceptable salts will be apparent to those skilled in the art and include those described in J. Pharm. Sci., 1977, 66, 1-19, such as acid addition salts formed with inorganic acids e.g. hydrochloric, hydrobromic, sulfuric, nitric or phosphoric acid; and organic acids e.g. succinic, maleic, acetic, fumaric, citric, tartaric, benzoic, p-toluenesulfonic, methanesulfonic or naphthalenesulfonic acid. Certain of the compounds of formula (I) may form acid addition salts with one or more equivalents of the acid. The present invention includes within its scope all possible stoichiometric and non-stoichiometric forms. Salts may also be prepared from pharmaceutically acceptable bases including inorganic bases and organic bases. Salts derived from inorganic bases include aluminum, ammonium, calcium, copper, ferric, ferrous, lithium, magnesium, manganic salts, manganous, potassium, sodium, zinc, and the like. Salts derived from pharmaceutically acceptable organic bases include salts of primary, secondary, and tertiary amines; substituted amines including naturally occurring substituted amines; and cyclic amines. Particular pharmaceutically acceptable organic bases include arginine, betaine, caffeine, choline, N,N′-dibenzylethylenediamine, diethylamine, 2-diethylaminoethanol, 2-dimethylaminoethanol, ethanolamine, ethylenediamine, N-ethyl-morpholine, N-ethylpiperidine, glucamine, glucosamine, histidine, hydrabamine, isopropylamine, lysine, methylglucamine, morpholine, piperazine, piperidine, procaine, purines, theobromine, triethylamine, trimethylamine, tripropylamine, tris(hydroxymethyl)aminomethane (TRIS, trometamol) and the like. Salts may also be formed from basic ion exchange resins, for example polyamine resins. When the compound of the present invention is basic, salts may be prepared from pharmaceutically acceptable acids, including inorganic and organic acids. Such acids include acetic, benzenesulfonic, benzoic, camphorsulfonic, citric, ethanesulfonic, ethanedisulfonic, fumaric, gluconic, glutamic, hydrobromic, hydrochloric, isethionic, lactic, maleic, malic, mandelic, methanesulfonic, mucic, pamoic, pantothenic, phosphoric, propionic, succinic, sulfuric, tartaric, p-toluenesulfonic acid, and the like.

Pharmaceutically acceptable acid addition salts may be prepared conventionally by reaction with the appropriate acid or acid derivative. Pharmaceutically acceptable salts with bases may be prepared conventionally by reaction with the appropriate inorganic or organic base.

The compounds of formula (I) may be prepared in crystalline or non-crystalline form, and, if crystalline, may optionally be hydrated or solvated. This invention includes within its scope stoichiometric hydrates or solvates as well as compounds containing variable amounts of water and/or solvent.

Included within the scope of the invention are all salts, solvates, hydrates, complexes, polymorphs, prodrugs, radiolabelled derivatives, stereoisomers and optical isomers of the compounds of formula (I).

The potencies and efficacies of the compounds of this invention for the S1P1 receptor can be determined by GTPγS assay performed on the human cloned receptor as described herein. Compounds of formula (I) have demonstrated agonist activity at the S1P1 receptor, using functional assays described herein.

Compounds of formula (I) and their pharmaceutically acceptable salts are therefore of use in the treatment of conditions or disorders which are mediated via the S1P1 receptor. In particular the compounds of formula (I) and their pharmaceutically acceptable salts are of use in the treatment of multiple sclerosis, autoimmune diseases, chronic inflammatory disorders, asthma, inflammatory neuropathies, arthritis, transplantation, Crohn's disease, ulcerative colitis, lupus erythematosis, psoriasis, ischemia-reperfusion injury, solid tumours, and tumour metastasis, diseases associated with angiogenesis, vascular diseases, pain conditions, acute viral diseases, inflammatory bowel conditions, insulin and non-insulin dependant diabetes.

Compounds of formula (I) and their pharmaceutically acceptable salts are therefore of use in the treatment of lupus erythematosis.

Compounds of formula (I) and their pharmaceutically acceptable salts are therefore of use in the treatment of psoriasis.

Compounds of formula (I) and their pharmaceutically acceptable salts are therefore of use in the treatment of multiple sclerosis.

Compounds of formula (I) and their pharmaceutically acceptable salts may also be of use in the treatment of Parkinson's Disease, Alzheimer's disease, Huntington's chorea, amyotrophic lateral sclerosis, spinal muscular atrophy, polyglutamine expansion disorders, vascular dementia, Down's syndrome, HIV dementia, dementia, ocular diseases including glaucoma, aged related macular degeneration, cataracts, traumatic eye injury, diabetic retinopathy, traumatic brain injury, stroke, tauopathies and hearing loss.

It is to be understood that “treatment” as used herein includes prophylaxis as well as alleviation of established symptoms.

Thus the invention also provides compounds of formula (I) or pharmaceutically acceptable salts thereof, for use as therapeutic substances, in particular in the treatment of the conditions or disorders mediated via the S1P1 receptor. In particular the invention provides a compound of formula (I) or a pharmaceutically acceptable salt thereof for use as a therapeutic substance in the treatment of multiple sclerosis, autoimmune diseases, chronic inflammatory disorders, asthma, inflammatory neuropathies, arthritis, transplantation, Crohn's disease, ulcerative colitis, lupus erythematosis, psoriasis, ischemia-reperfusion injury, solid tumours, and tumour metastasis, diseases associated with angiogenesis, vascular diseases, pain conditions, acute viral diseases, inflammatory bowel conditions, insulin and non-insulin dependant diabetes.

Compounds of formula (I) and their pharmaceutically acceptable salts are of use as therapeutic substances in the treatment of lupus erythematosis.

Compounds of formula (I) and their pharmaceutically acceptable salts are of use as therapeutic substances in the treatment of psoriasis.

Compounds of formula (I) and their pharmaceutically acceptable salts are of use as therapeutic substances in the treatment of multiple sclerosis.

The invention further provides a method of treatment of conditions or disorders in mammals including humans which can be mediated via the S1P1 receptor, which comprises administering to the sufferer a therapeutically safe and effective amount of a compound of formula (I) or a pharmaceutically acceptable salt thereof. In particular the invention provides a method of treatment of multiple sclerosis, autoimmune diseases, chronic inflammatory disorders, asthma, inflammatory neuropathies, arthritis, transplantation, Crohn's disease, ulcerative colitis, lupus erythematosis, psoriasis, ischemia-reperfusion injury, solid tumours, and tumour metastasis, diseases associated with angiogenesis, vascular diseases, pain conditions, acute viral diseases, inflammatory bowel conditions, insulin and non-insulin dependant diabetes, which comprises administering to the sufferer a therapeutically safe and effective amount of a compound of formula (I) or a pharmaceutically acceptable salt thereof.

The invention provides a method of treatment of lupus erythematosis, which comprises administering to the sufferer a therapeutically safe and effective amount of a compound of formula (I) or a pharmaceutically acceptable salt thereof.

The invention provides a method of treatment of psoriasis, which comprises administering to the sufferer a therapeutically safe and effective amount of a compound of formula (I) or a pharmaceutically acceptable salt thereof.

The invention provides a method of treatment of multiple sclerosis, which comprises administering to the sufferer a therapeutically safe and effective amount of a compound of formula (I) or a pharmaceutically acceptable salt thereof.

In another aspect, the invention provides for the use of a compound of formula (I) or a pharmaceutically acceptable salt thereof in the manufacture of a medicament for use in the treatment of the conditions or disorders mediated via the S1P1 receptor.

In particular the invention provides a compound of formula (I) or a pharmaceutically acceptable salt thereof for use in the manufacture of a medicament for use in the treatment of multiple sclerosis, autoimmune diseases, chronic inflammatory disorders, asthma, inflammatory neuropathies, arthritis, transplantation, Crohn's disease, ulcerative colitis, lupus erythematosis, psoriasis, ischemia-reperfusion injury, solid tumours, and tumour metastasis, diseases associated with angiogenesis, vascular diseases, pain conditions, acute viral diseases, inflammatory bowel conditions, insulin and non-insulin dependant diabetes.

Compounds of formula (I) and their pharmaceutically acceptable salts are of use in the manufacture of a medicament for use in the treatment of lupus erythematosis.

Compounds of formula (I) and their pharmaceutically acceptable salts are of use in the manufacture of a medicament for use in the treatment of psoriasis.

Compounds of formula (I) and their pharmaceutically acceptable salts are of use in the manufacture of a medicament for use in the treatment of multiple sclerosis.

In order to use the compounds of formula (I) and pharmaceutically acceptable salts thereof in therapy, they will normally be formulated into a pharmaceutical composition in accordance with standard pharmaceutical practice. The present invention also provides a pharmaceutical composition, which comprises a compound of formula (I) or a pharmaceutically acceptable salt thereof, and a pharmaceutically acceptable carrier or excipient.

In a further aspect, the present invention provides a process for preparing a pharmaceutical composition, the process comprising mixing a compound of formula (I) or a pharmaceutically acceptable salt thereof and a pharmaceutically acceptable carrier or excipient.

A pharmaceutical composition of the invention, which may be prepared by admixture, suitably at ambient temperature and atmospheric pressure, is usually adapted for oral, parenteral or rectal administration and, as such, may be in the form of tablets, capsules, oral liquid preparations, powders, granules, lozenges, reconstitutable powders, injectable or infusible solutions or suspensions or suppositories. Orally administrable compositions are generally preferred.

Tablets and capsules for oral administration may be in unit dose form, and may contain conventional excipients, such as binding agents (e.g. pregelatinised maize starch, polyvinylpyrrolidone or hydroxypropyl methylcellulose); fillers (e.g. lactose, microcrystalline cellulose or calcium hydrogen phosphate); tabletting lubricants (e.g. magnesium stearate, talc or silica); disintegrants (e.g. potato starch or sodium starch glycollate); and acceptable wetting agents (e.g. sodium lauryl sulphate). The tablets may be coated according to methods well known in normal pharmaceutical practice.

Oral liquid preparations may be in the form of, for example, aqueous or oily suspension, solutions, emulsions, syrups or elixirs, or may be in the form of a dry product for reconstitution with water or other suitable vehicle before use. Such liquid preparations may contain conventional additives such as suspending agents (e.g. sorbitol syrup, cellulose derivatives or hydrogenated edible fats), emulsifying agents (e.g. lecithin or acacia), non-aqueous vehicles (which may include edible oils e.g. almond oil, oily esters, ethyl alcohol or fractionated vegetable oils), preservatives (e.g. methyl or propyl-p-hydroxybenzoates or sorbic acid), and, if desired, conventional flavourings or colorants, buffer salts and sweetening agents as appropriate. Preparations for oral administration may be suitably formulated to give controlled release of the active compound.

For parenteral administration, fluid unit dosage forms are prepared utilising a compound of the invention or pharmaceutically acceptable salts thereof and a sterile vehicle. Formulations for injection may be presented in unit dosage form e.g. in ampoules or in multi-dose, utilising a compound of the invention or pharmaceutically acceptable derivatives thereof and a sterile vehicle, optionally with an added preservative. The compositions may take such forms as suspensions, solutions or emulsions in oily or aqueous vehicles, and may contain formulatory agents such as suspending, stabilising and/or dispersing agents. Alternatively, the active ingredient may be in powder form for constitution with a suitable vehicle, e.g. sterile pyrogen-free water, before use. The compound, depending on the vehicle and concentration used, can be either suspended or dissolved in the vehicle. In preparing solutions, the compound can be dissolved for injection and filter sterilised before filling into a suitable vial or ampoule and sealing. Advantageously, adjuvants such as a local anaesthetic, preservatives and buffering agents are dissolved in the vehicle. To enhance the stability, the composition can be frozen after filling into the vial and the water removed under vacuum. Parenteral suspensions are prepared in substantially the same manner, except that the compound is suspended in the vehicle instead of being dissolved, and sterilisation cannot be accomplished by filtration. The compound can be sterilised by exposure to ethylene oxide before suspension in a sterile vehicle. Advantageously, a surfactant or wetting agent is included in the composition to facilitate uniform distribution of the compound.

Lotions may be formulated with an aqueous or oily base and will in general also contain one or more emulsifying agents, stabilising agents, dispersing agents, suspending agents, thickening agents, or colouring agents. Drops may be formulated with an aqueous or non-aqueous base also comprising one or more dispersing agents, stabilising agents, solubilising agents or suspending agents. They may also contain a preservative.

The compounds of formula (I) or pharmaceutically acceptable salts thereof may also be formulated in rectal compositions such as suppositories or retention enemas, e.g. containing conventional suppository bases such as cocoa butter or other glycerides.

The compounds of formula (I) or pharmaceutically acceptable salts thereof may also be formulated as depot preparations. Such long acting formulations may be administered by implantation (for example subcutaneously or intramuscularly) or by intramuscular injection. Thus, for example, the compounds of the invention may be formulated with suitable polymeric or hydrophobic materials (for example as an emulsion in an acceptable oil) or ion exchange resins, or as sparingly soluble derivatives, for example, as a sparingly soluble salt.

For intranasal administration, the compounds of formula (I) or pharmaceutically acceptable salts thereof, may be formulated as solutions for administration via a suitable metered or unitary dose device or alternatively as a powder mix with a suitable carrier for administration using a suitable delivery device. Thus compounds of formula (I) or pharmaceutically acceptable salts thereof may be formulated for oral, buccal, parenteral, topical (including ophthalmic and nasal), depot or rectal administration or in a form suitable for administration by inhalation or insufflation (either through the mouth or nose).

The compounds of formula (I) or pharmaceutically acceptable salts thereof may be formulated for topical administration in the form of ointments, creams, gels, lotions, pessaries, aerosols or drops (e.g. eye, ear or nose drops). Ointments and creams may, for example, be formulated with an aqueous or oily base with the addition of suitable thickening and/or gelling agents. Ointments for administration to the eye may be manufactured in a sterile manner using sterilised components.

The composition may contain from 0.1% to 99% by weight, preferably from 10 to 60% by weight, of the active material, depending on the method of administration. The dose of the compound used in the treatment of the aforementioned disorders will vary in the usual way with the seriousness of the disorders, the weight of the sufferer, and other similar factors. However, as a general guide suitable unit doses may be 0.05 to 1000 mg, 1.0 to 500 mg or 1.0 to 200 mg and such unit doses may be administered more than once a day, for example two or three times a day.

Compounds of formula (I) or pharmaceutically acceptable salts thereof may be used in combination preparations, in combination with other active ingredients. For example, the compounds of the invention may be used in combination with cyclosporin A, methotrexate, steriods, rapamycin, proinflammatory cytokine inhibitors, immunomodulators including biologicals or other therapeutically active compounds.

The subject invention also includes isotopically-labeled compounds, which are identical to those recited in formulas I and following, but for the fact that one or more atoms are replaced by an atom having an atomic mass or mass number different from the atomic mass or mass number usually found; in nature. Examples of isotopes that can be incorporated into compounds of the invention include isotopes of hydrogen, carbon, nitrogen, oxygen, phosphorous, fluorine, iodine, and chlorine, such as ³H, ¹¹C, ¹⁴C, ¹⁸F, ¹²³I and ¹²⁵I.

Compounds of the present invention and pharmaceutically acceptable salts of said compounds that contain the aforementioned isotopes and/or other isotopes of other atoms are within the scope of the present invention. Isotopically-labeled compounds of the present invention, for example those into which radioactive isotopes such as ³H, ¹⁴C are incorporated, are useful in drug and/or substrate tissue distribution assays. Tritiated, i.e., ³H, and carbon-14, i.e., ¹⁴C, isotopes are particularly preferred for their ease of preparation and detectability. ¹¹C and ⁸F isotopes are particularly useful in PET (positron emission tomography), and ¹²⁵I isotopes are particularly useful in SPECT (single photon emission computerized tomography), all useful in brain imaging. Further, substitution with heavier isotopes such as deuterium, i.e., ²H, can afford certain therapeutic advantages resulting from greater metabolic stability, for example increased in vivo half-life or reduced dosage requirements and, hence, may be preferred in some circumstances. Isotopically labelled compounds of formula (I) and following of this invention can generally be prepared by carrying out the procedures disclosed in the Schemes and/or in the Examples below, by substituting a readily available isotopically labelled reagent for a non-isotopically labeled reagent.

In a further aspect, this invention provides processes for preparation of a compound of formula (I).

All publications, including but not limited to patents and patent applications, cited in this specification are herein incorporated by reference as if each individual publication were specifically and individually indicated to be incorporated by reference herein as though fully set forth.

The following Descriptions and Examples illustrate the preparation of compounds of the invention.

### ABBREVIATIONS

g—grams
mg—milligrams
ml—millilitres
μl—microlitres
DCM—dichloromethane
DIPEA—diisopropylethylamine
DME—1,2-bis(methyloxy)ethane

### DMF—N,N-dimethylformamide

DMSO—dimethylsulphoxide
D₆DMSO—deuterated dimethylsulphoxide
EDC—N-(3-Dimethylaminopropyl)-N′-ethylcarbodiimide hydrochloride

### HOBT—Hydroxybenzotriazole

THF—tetrahydrofuran
TFA—trifluoroacetic acid
° C.—degrees Celsius

### M—Molar

H—proton
s—singlet
d—doublet
t—triplet
q—quartet
m—multiplet
MHz—megahertz

### LCMS—Liquid Chromatography Mass Spectrometry

MS—mass spectrometry
MH⁺—mass ion+H+
MDAP—mass directed automated preparative liquid chromatography.
SCX—solid phase cation exchange SPE
SPE—solid phase ion exchange cartridge (supplied by (solute or Varian)

### LCMS Methodology

All the Examples were analysed using one of the following LCMS methods except for Examples 204 and 205 and intermediates from Preparations 195 to 210.

### Method Formate

### LC Conditions

The HPLC analysis was conducted on an Acquity HPLC BEH C18 column (50 mm×2.1 mm, i.d. 1.7 μm packing diameter) at 40° C.

The solvents employed were:

A=0.1% v/v solution of formic acid in water

B=0.1% v/v solution of formic acid in acetonitrile

The gradient employed was:

| Time (min) | Flow rate (ml/min) | % A | % B |
|---|---|---|---|
| 0 | 1 | 99 | 1 |
| 1.5 | 1 | 3 | 97 |
| 1.9 | 1 | 3 | 97 |
| 2.0 | 1 | 0 | 100 |

The UV detection was a summed signal from wavelength of 210 nm to 350 nm.

### MS Conditions

### MS: Waters: ZQ

Ionisation mode: Alternate-scan positive and negative electrospray
Scan range: 100 to 1000 AMU
Scan time: 0.27 sec
Inter scan delay: 0.10 sec

### Method HpH

### LC Conditions

The UPLC analysis was conducted on an Acquity HPLC BEH C18 column (50 mm×2.1 mm, i.d. 1.7 μm packing diameter) at 40° C.

The solvents employed were:

A=10 mM ammonium hydrogen carbonate in water adjusted to pH10 with ammonia solution

B=acetonitrile

The gradient employed was:

| Time (min) | Flow rate (ml/min) | % A | % B |
|---|---|---|---|
| 0 | 1 | 99 | 1 |
| 1.5 | 1 | 3 | 97 |
| 1.9 | 1 | 3 | 97 |
| 2.0 | 1 | 0 | 100 |

The UV detection was a summed signal from wavelength of 210 nm to 350 nm.

### MS Conditions

### MS Waters: ZQ

Ionisation mode: Alternate-scan positive and negative electrospray
Scan range: 100 to 1000 AMU
Scan time: 0.27 sec
Inter scan delay: 0.10 sec

### M DAP Methodology

### Method Formate

### LC Conditions

The HPLC analysis was conducted on either a Sunfire C18 column (100 mm×1.9 mm,i.d 5 μm packing diameter) or a Sunfire C18 column (150 mm×30 mm, i.d. 5 μm packing diameter) at ambient temperature.

The solvents employed were:

A=0.1% v/v solution of formic acid in water

B=0.1% v/v solution of formic acid in acetonitrile

Run as a gradient over either 15 or 25 min with a flow rate of 20 ml/min (100 mm×1.9 mm,i.d 5 μm packing diameter) or 40 ml/min (150 mm×30 mm, i.d. 5 μm packing diameter).

The UV detection was a summed signal from wavelength of 210 nm to 350 nm.

### MS Conditions

### MS Waters: ZQ

Ionisation mode: Alternate-scan positive and negative electrospray
Scan range: 100 to 1000 AMU
Scan time: 0.50 sec
Inter scan delay: 0.20 sec

### General Chemistry Section

The methods described below are given for illustrative purposes. Intermediates in the preparation of the examples may not necessarily have been prepared from the specific batches described.

### Preparation 1

### 5-(5-Amino-1,3,4-thiadiazol-2-yl)-2-[(1-methylethyl)oxy]benzonitrile

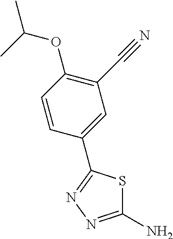

3-Cyano-4-[(1-methylethyl)oxy]benzoic acid (20.9 g, 100 mmol, Biopharma) and hydrazinecarbothioamide (13.9 g, 150 mmol, Aldrich) were combined and phosphorus oxychloride (90 g, 590 mmol) added to the mixture. This was heated at 90° C. for 3 h, cooled to room temperature and added in small portions to sodium hydroxide (5M) cooled with an ice bath and maintaining a reaction temperature <35° C. The mixture was basified to pH 10 and stirred for 30 min. The solid was collected by filtration, dissolved in DCM (1 l) and methanol (50 ml) and the solution washed with water (500 ml). The organic phase was dried and concentrated to give 5-(5-amino-1,3,4-thiadiazol-2-yl)-2-[(1-methylethyl)oxy]benzonitrile (26.3 g, 99%) as pale yellow solid which was used in the next step (Preparation 2) without further purification.

LCMS (Method formate): Retention time 0.86 min, MH⁺=261

### Preparation 2

### 5-(5-Bromo-1,3,4-thiadiazol-2-yl)-2-[(1-methylethyl)oxy]benzonitrile

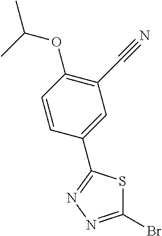

Cupric bromide (19.6 g, 88 mmol) and tert-butyl nitrite (10.4 ml, 88 mmol) were dissolved in acetonitrile (400 ml) and the resulting mixture was stirred for 10 min. 5-(5-Amino-1,3,4-thiadiazol-2-yl)-2-[(1-methylethyl)oxy]benzonitrile (Preparation 1) (13 g, 40 mmol) was then added in small portions over 30 min. The mixture was stirred for 1 h at room temperature, then at 70° C. for 2 h, cooled to room temperature and concentrated in vacuo. The residue was stirred at reflux in ethyl acetate (600 ml) and methanol (50 ml) for 1 h, filtered through a pad of silica (5 cm) and the silica washed with ethyl acetate (200 ml). The combined organic phases were washed hydrochloric acid (1M, 300 ml), dried and concentrated. The residue was dissolved in DCM (100 ml) and loaded onto a silica cartridge (330 g) elution with an ethyl acetate/cyclohexane gradient (0-100% ethyl acetate) gave 5-(5-bromo-1,3,4-thiadiazol-2-yl)-2-[(1-methylethyl)oxy]benzonitrile (8.8 g, 68%) as a pale yellow solid.

LCMS (Method formate): Retention time 1.13 min, MH⁺=324/326.

### Preparation 3

### 2-[(1-methylethyl)oxy]-5-[3-(5-methyl-1,2,3,4-tetrahydro-6-isoquinolinyl)-1,2,4-oxadiazol-5-yl]-3-pyridinecarbonitrile trifluoroacetate

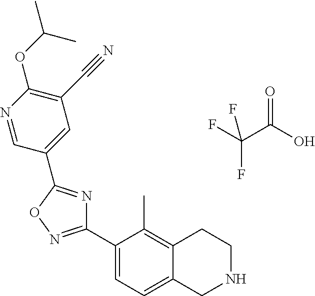

Trifluoroacetic acid (0.91 ml, 12 mmol) was added dropwise to a solution of 1,1-dimethylethyl 6-(5-{5-cyano-6-[(1-methylethyl)oxy]-3-pyridinyl}-1,2,4-oxadiazol-3-yl)-5-methyl-3,4-dihydro-2(1H)-isoquinolinecarboxylate (Preparation 4) (1.10 g, 2.4 mmol) in DCM (5 ml) at 0° C., the mixture allowed to warm to room temperature and stirred for 16 h. The volatiles were evaporated, the resulting solid triturated with diethyl ether (2×5 ml) and the solid isolated by filtration to give 2-[(1-methylethyl)oxy]-5-[3-(5-methyl-1,2,3,4-tetrahydro-6-isoquinolinyl)-1,2,4-oxadiazol-5-yl]-3-pyridinecarbonitrile trifluoroacetate as a colourless solid (1.39 g).

LCMS (Method formate): Retention time 0.96 min, MH⁺=376

### Preparation 4

### 1,1-dimethylethyl 6-(5-{5-cyano-6-[(1-methylethyl)oxy]-3-pyridinyl}-1,2,4-oxadiazol-3-yl)-5-methyl-3,4-dihydro-2(1H)-isoquinolinecarboxylate

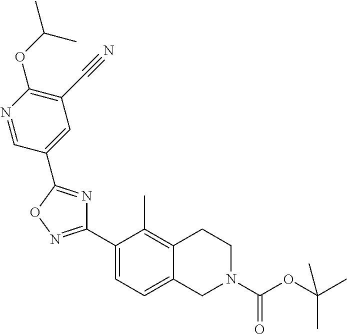

5-Cyano-6-[(1-methylethyl)oxy]-3-pyridinecarbonyl chloride (Preparation 98) (81 mg, 0.36 mmol) was added portionwise to a suspension of 1,1-dimethylethyl 6-[(hydroxyamino)(imino)methyl]-5-methyl-3,4-dihydro-2(1H)-isoquinolinecarboxylate (Preparation 23) (105 mg, 0.34 mmol, WO2009080724) in toluene (2 ml) and pyridine (2 ml) at room temperature under nitrogen and the mixture stirred for 20 min. The mixture was then heated at 120° C. for 2 h.

5-Cyano-6-[(1-methylethyl)oxy]-3-pyridinecarbonyl chloride (Preparation 98) (660 mg, 2.9 mmol) was added portionwise to a suspension of 1,1-dimethylethyl 6-[(hydroxyamino)(imino)methyl]-5-methyl-3,4-dihydro-2(1H)-isoquinolinecarboxylate (Preparation 23) (855 mg, 2.8 mmol, WO2009080724) in toluene (10 ml) and pyridine (10 ml) at room temperature under nitrogen and the mixture stirred for 20 min. The mixture was heated at 120° C. for 2 h. The mixture was combined with the reaction above and evaporated to dryness. The residue was dissolved in DCM and loaded onto a silica cartridge. The cartridge was eluted with an ethyl acetate/cyclohexane gradient (0-50%). The appropriate fractions were combined and evaporated in vacuo to give 1,1-dimethylethyl 6-(5-{5-cyano-6-[(1-methylethyl)oxy]-3-pyridinyl}-1,2,4-oxadiazol-3-yl)-5-methyl-3,4-dihydro-2(1H)-isoquinolinecarboxylate (1.24 g) as a colourless foam.

LCMS (Method formate): Retention time 1.54 min, [2M+H]⁺=951

### Preparation 5

### 5-{3-[2-(2,2-dimethyl-1,3-dioxan-5-yl)-5-methyl-1,2,3,4-tetrahydro-6-isoquinolinyl]-1,2,4-oxadiazol-5-yl}-2-[(1-methylethyl)oxy]-3-pyridinecarbonitrile

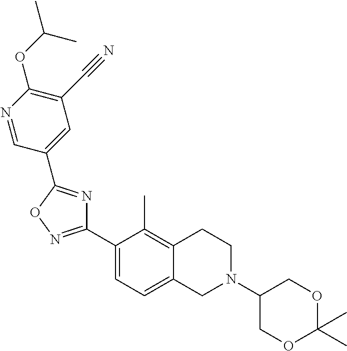

2,2-Dimethyl-1,3-dioxan-5-one (199 mg, 1.5 mmol) was added to a stirred solution of 2-[(1-methylethyl)oxy]-5-[3-(5-methyl-1,2,3,4-tetrahydro-6-isoquinolinyl)-1,2,4-oxadiazol-5-yl]-3-pyridinecarbonitrile trifluoroacetate (Preparation 3) (150 mg, 0.31 mmol) in 1,2-dichloroethane (2 ml) and THF (2 ml). The reaction mixture was stirred at room temperature for 30 min then sodium triacetoxyborohydride (325 mg, 1.5 mmol) was added. Stirring at room temperature was continued for 4 h. Further portions of 2,2-dimethyl-1,3-dioxan-5-one (199 mg, 1.5 mmol) and sodium triacetoxyborohydride (325 mg, 1.5 mmol) were added and stirring continued overnight. Further portions of 2,2-dimethyl-1,3-dioxan-5-one (199 mg, 1.5 mmol) and sodium triacetoxyborohydride (325 mg, 1.5 mmol) were added and stirring continued for 6 h. Saturated sodium hydrogen carbonate (10 ml) was added and the mixture extracted with ethyl acetate (2×10 ml). The combined organic extracts were washed with water and brine, dried and evaporated. The residue was chromatographed (ethyl acetate/isohexane, 10-40%) and trituration of the residue with diethyl ether give 5-{3-[2-(2,2-dimethyl-1,3-dioxan-5-yl)-5-methyl-1,2,3,4-tetrahydro-6-isoquinolinyl]-1,2,4-oxadiazol-5-yl}-2-[(1-methylethyl)oxy]-3-pyridinecarbonitrile as a colourless solid (30 mg).

LCMS (Method formate): Retention time 1.04 min, MH⁺=490

### Preparation 6

### 5-[3-(2,3,4,5-tetrahydro-1H-3-benzazepin-7-yl)-1,2,4-oxadiazol-5-yl]-2-[(2,2,2-trifluoroethyl)oxy]benzonitrile hydrochloride

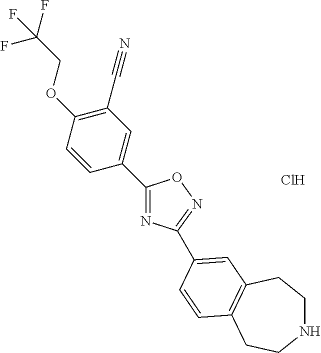

A solution of 1,1-dimethylethyl 7-(5-{3-cyano-4-[(2,2,2-trifluoroethyl)oxy]phenyl}-1,2,4-oxadiazol-3-yl)-1,2,4,5-tetrahydro-3H-3-benzazepine-3-carboxylate (Preparation 7) (796 mg, 1.5 mmol) in 1,4-dioxane (10 ml) was treated with hydrogen chloride in 1,4-dioxane (4N, 10 ml, 40 mmol) and the resulting mixture was stirred for 4 h at room temperature then concentrated in vacuo. The residue was co-evaporated with diethyl ether then dried under vacuum for 16 h to give 5-[3-(2,3,4,5-tetrahydro-1H-3-benzazepin-7-yl)-1,2,4-oxadiazol-5-yl]-2-[(2,2,2-trifluoroethyl)oxy]benzonitrile hydrochloride (730 mg, 105%) as a white solid.

LCMS (Method HpH): Retention time 1.16 min, MH⁺=415

### Preparation 7

### 1,1-dimethylethyl 7-(5-{3-cyano-4-[(2,2,2-trifluoroethyl)oxy]phenyl}-1,2,4-oxadiazol-3-yl)-1,2,4,5-tetrahydro-3H-3-benzazepine-3-carboxylate

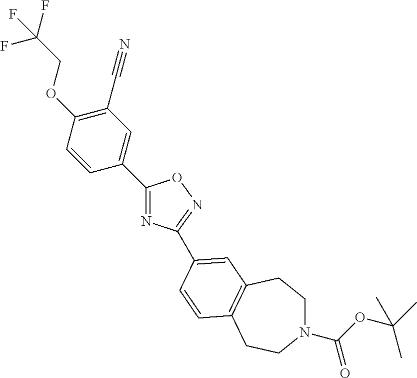

To a solution of 1,1-dimethylethyl 7-[5-(3-cyano-4-hydroxyphenyl)-1,2,4-oxadiazol-3-yl]-1,2,4,5-tetrahydro-3H-3-benzazepine-3-carboxylate (Preparation 8) (800 mg, 1.9 mmol) in DMF (20 ml) at room temperature under nitrogen was added potassium carbonate (380 mg, 2.8 mmol) then 2,2,2-trifluoroethyl trifluoromethanesulfonate (558 mg, 2.4 mmol) as a solution in DMF (2 ml) and the mixture was stirred at 50° C. for 1 h then cooled to room temperature. The reaction was filtered, the residue washed with ethyl acetate and the combined filtrate and washings concentrated in vacuo. The residue was dissolved in ethyl acetate, washed with brine, dried (MgSO₄) and concentrated in vacuo. The residue was purified by chromatography using an ethyl acetate/cyclohexane gradient to give 1,1-dimethylethyl 7-(5-{3-cyano-4-[(2,2,2-trifluoroethyl)oxy]phenyl}-1,2,4-oxadiazol-3-yl)-1,2,4,5-tetrahydro-3H-3-benzazepine-3-carboxylate (796 mg, 84%) as a white solid.

LCMS (Method HpH): Retention time 1.48 min, MH⁺=459

### Preparation 8

### 1,1-dimethylethyl 7-[5-(3-cyano-4-hydroxyphenyl)-1,2,4-oxadiazol-3-yl]-1,2,4,5-tetrahydro-3H-3-benzazepine-3-carboxylate

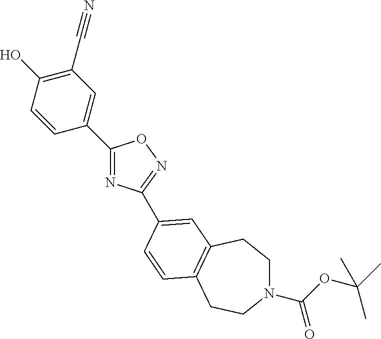

A suspension of 1,1-dimethylethyl 7-(5-{3-cyano-4-[(phenylmethyl)oxy]phenyl}-1,2,4-oxadiazol-3-yl)-1,2,4,5-tetrahydro-3H-3-benzazepine-3-carboxylate (Preparation 9) (2.11 g, 4.0 mmol) in toluene (40 ml) was warmed until everything was in solution then cooled to room temperature and treated with palladium on carbon (10% w/w, 50% wet, 450 mg, 0.42 mmol). The suspension was stirred under hydrogen (1 bar) for 16 h. Palladium on carbon (10% w/w, 50% wet, 450 mg, 0.42 mmol) was added and the reaction hydrogenated for a further 24 h. The reaction was filtered through Celite™, the residue washed with ethanol and the combined filtrate and washings concentrated in vacuo. The resulting foam was purified by chromatography eluting with an ethyl acetate/cyclohexane gradient and the column washed to give 1,1-dimethylethyl 7-[5-(3-cyano-4-hydroxyphenyl)-1,2,4-oxadiazol-3-yl]-1,2,4,5-tetrahydro-3H-3-benzazepine-3-carboxylate as a white solid (800 mg).

1H NMR (CDCl₃): δ H 8.37 (1H, s), 8.22 (1H, d), 7.90 (2H, m), 7.26 (2H, m), 7.13 (1H, d), 3.61 (4H, m), 3.00 (4H, m), 1.50 (9H, s).

### Preparation 9

### 1,1-dimethylethyl 7-(5-{3-cyano-4-[(phenylmethyl)oxy]phenyl}-1,2,4-oxadiazol-3-yl)-1,2,4,5-tetrahydro-3H-3-benzazepine-3-carboxylate

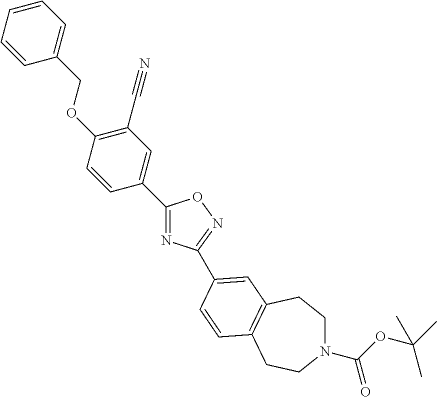

To a solution of benzyl alcohol (0.78 ml, 7.5 mmol) in THF (20 ml) at room temperature under nitrogen was added sodium hydride (60% w/w in oil, 0.28 g, 7.0 mmol) and after 5 min a solution of 1,1-dimethylethyl 7-[5-(3-cyano-4-fluorophenyl)-1,2,4-oxadiazol-3-yl]-1,2,4,5-tetrahydro-3H-3-benzazepine-3-carboxylate (Preparation 68) (2.52 g, 5.8 mmol) in THF (20 ml) dropwise. The solution was stirred for ca 15 minutes and most of the solvent evaporated in vacuo. The residue was dissolved in ethyl acetate and the organic phase washed with brine. The aqueous phase was extracted and the combined organic phases washed with water then brine. The solution was dried (MgSO₄) and concentrated in vacuo. Purification of the residue by chromatography using an ethyl acetate/cyclohexane gradient gave 1,1-dimethylethyl 7-(5-{3-cyano-4-[(phenylmethyl)oxy]phenyl}-1,2,4-oxadiazol-3-yl)-1,2,4,5-tetrahydro-3H-3-benzazepine-3-carboxylate (2.45 g, 81%) as a white solid.

LCMS (Method HpH): Retention time 1.58 min, MH⁺=467

### Preparation 10

### [6-(5-{3-chloro-4-[(1-methylethyl)oxy]phenyl}-1,2,4-oxadiazol-3-yl)-5-methyl-3,4-dihydro-2(1H)-isoquinolinyl]acetic acid

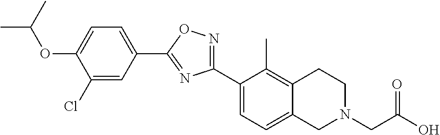

To a suspension of methyl [6-(5-{3-chloro-4-[(1-methylethyl)oxy]phenyl}-1,2,4-oxadiazol-3-yl)-5-methyl-3,4-dihydro-2(1H)-isoquinolinyl]acetate (Preparation 11) (510 mg, 1.119 mmol) in ethanol (6 ml) and methanol (6 ml) was added aqueous sodium hydroxide (2N, 1.12 ml, 2.2 mmol) and the resulting mixture was stirred for 3 h, then left in a freezer overnight. The reaction was concentrated in vacuo, diluted with water (10 ml) and treated with acetic acid (0.26 ml, 4.5 mmol). Ethyl acetate was added, the suspension filtered, and the separated organic phase dried (MgSO₄) and concentrated in vacuo. The residue was combined with the previously isolated solid to give [6-(5-{3-chloro-4-[(1-methylethyl)oxy]phenyl}-1,2,4-oxadiazol-3-yl)-5-methyl-3,4-dihydro-2(1H)-isoquinolinyl]acetic acid (392 mg, 79%) as a white solid.

LCMS (Method HpH): Retention time 1.03 min, MH⁺=442

### Preparation 11

### methyl [6-(5-{3-chloro-4-[(1-methylethyl)oxy]phenyl}-1,2,4-oxadiazol-3-yl)-5-methyl-3,4-dihydro-2(1H)-isoquinolinyl]acetate

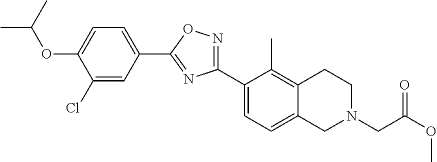

A suspension of 6-(5-{3-chloro-4-[(1-methylethyl)oxy]phenyl}-1,2,4-oxadiazol-3-yl)-5-methyl-1,2,3,4-tetrahydroisoquinoline hydrochloride (Preparation 12) (715 mg, 1.7 mmol) and potassium carbonate (705 mg, 5.1 mmol) in acetonitrile (16 ml) at room temperature under nitrogen was treated with methyl bromoacetate (0.17 ml, 1.9 mmol) and the resulting mixture was stirred at 50° C. for 1 h then cooled to room temperature and most of the solvent evaporated in vacuo. The residue was partitioned between ethyl acetate and saturated sodium hydrogen carbonate. The aqueous phase was extracted with ethyl acetate (x2) and the combined organic phases were washed with brine/saturated sodium hydrogen carbonate, dried (MgSO₄) and concentrated in vacuo. The resulting yellow foam was purified by chromatography eluting with an ethyl acetate/cyclohexane gradient to give methyl [6-(5-{3-chloro-4-[(1-methylethyl)oxy]phenyl}-1,2,4-oxadiazol-3-yl)-5-methyl-3,4-dihydro-2(1H)-isoquinolinyl]acetate (530 mg, 68%) as a white solid.

LCMS (Method HpH): Retention time 1.53 min, MH⁺=456

### Preparation 12

### 6-(5-{3-chloro-4-[(1-methylethyl)oxy]phenyl}-1,2,4-oxadiazol-3-yl)-5-methyl-1,2,3,4-tetrahydroisoquinoline hydrochloride

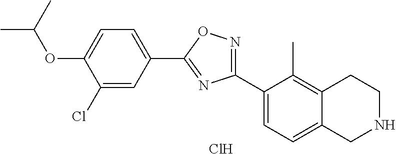

To a solution of 1,1-dimethylethyl 6-(5-{3-chloro-4-[(1-methylethyl)oxy]phenyl}-1,2,4-oxadiazol-3-yl)-5-methyl-3,4-dihydro-2(1H)-isoquinolinecarboxylate (1.85 g, 3.8 mmol, WO 2009080724) in 1,4-dioxane (10 ml) at room temperature under nitrogen was added slowly hydrogen chloride in 1,4-dioxane (4N, 30 ml, 120 mmol) and the resulting mixture was stirred at room temperature for 3.5 h. Removal of the solvent and co-evaporation of the residue with diethyl ether gave 6-(5-{3-chloro-4-[(1-methylethyl)oxy]phenyl}-1,2,4-oxadiazol-3-yl)-5-methyl-1,2,3,4-tetrahydroisoquinoline hydrochloride (1.65 g, 103%) as a white solid.

LCMS (Method HpH): Retention time 1.43 min, MH⁺=384

### Preparation 13

### 1,1-dimethylethyl {2-[6-(5-{5-cyano-6-[(1-methylethyl)oxy]-3-pyridinyl}-1,2,4-oxadiazol-3-yl)-5-methyl-3,4-dihydro-2(1H)-isoquinolinyl]-2-oxoethyl}carbamate

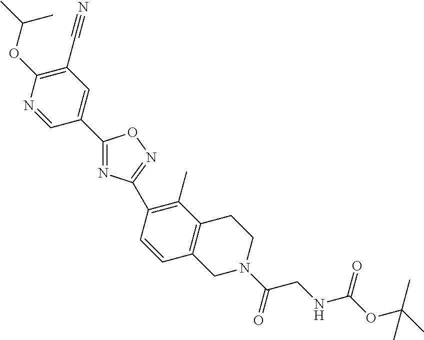

A mixture of N-Bocglycine (60 mg, 0.34 mmol), N-ethylmorpholine (86 μl, 0.68 mmol), hydroxybenzotriazole hydrate (62 mg, 0.41 mmol), EDC (78 mg, 0.41 mmol) and 2-[(1-methylethyl)oxy]-5-[3-(5-methyl-1,2,3,4-tetrahydro-6-isoquinolinyl)-1,2,4-oxadiazol-5-yl]-3-pyridinecarbonitrile trifluoroacetate (Preparation 3) (200 mg, 0.41 mmol) in DMF (3 ml) was stirred at room temperature for 6 h. The reaction mixture was partitioned between ethyl acetate (20 ml) and saturated sodium hydrogen carbonate (20 ml). The organic phase was separated, washed with water and brine, dried and evaporated. The residue was chromatographed (methanol/DCM, 0-4%) to give 1,1-dimethylethyl {2-[6-(5-{5-cyano-6-[(1-methylethyl)oxy]-3-pyridinyl}-1,2,4-oxadiazol-3-yl)-5-methyl-3,4-dihydro-2(1H)-isoquinolinyl]-2-oxoethyl}carbamate as a colourless oil (170 mg).

LCMS (Method formate): Retention time 1.36 min, MH⁺=533

### Preparation 14

### 2-[(1-methylethyl)oxy]-5-[3-(8-methyl-2,3,4,5-tetrahydro-1H-3-benzazepin-7-yl)-1,2,4-oxadiazol-5-yl]benzonitrile trifluoroacetate

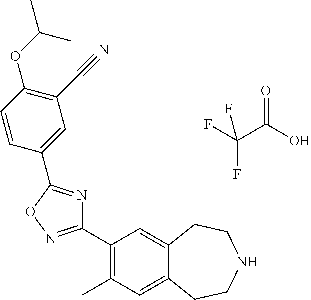

Trifluoroacetic acid (5 ml) was added dropwise over 5 min to a stirred solution of 1,1-dimethylethyl 7-(5-{3-cyano-4-[(1-methylethyl)oxy]phenyl}-1,2,4-oxadiazol-3-yl)-8-methyl-1,2,4,5-tetrahydro-3H-3-benzazepine-3-carboxylate (Preparation 15) (1.15 g, 2.4 mmol) in DCM (20 ml). After complete addition the reaction mixture was stirred at room temperature for 1 h. The solvent was evaporated and the residue was re-evaporated from toluene (x2). Trituration of the residue with diethyl ether gave 2-[(1-methylethyl)oxy]-5-[3-(8-methyl-2,3,4,5-tetrahydro-1H-3-benzazepin-7-yl)-1,2,4-oxadiazol-5-yl]benzonitrile trifluoroacetate as a colourless solid (1.0 g).

LCMS (Method formate): Retention time 0.93 min, MH⁺=389

### Preparation 15

### 1,1-dimethylethyl 7-(5-{3-cyano-4-[(1-methylethyl)oxy]phenyl}-1,2,4-oxadiazol-3-yl)-8-methyl-1,2,4,5-tetrahydro-3H-3-benzazepine-3-carboxylate

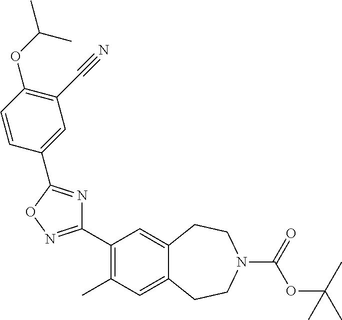

3-Cyano-4-[(1-methylethyl)oxy]benzoyl chloride (1.02 g, 4.6 mmol, WO2009080730/WO2008128951) was added portionwise to a stirred solution of 1,1-dimethylethyl 7-[(hydroxyamino)(imino)methyl]-8-methyl-1,2,4,5-tetrahydro-3H-3-benzazepine-3-carboxylate (Preparation 16) (1.39 g, 4.4 mmol) in toluene (20 ml) and pyridine (10 ml).

The reaction mixture was stirred at room temperature for 10 min and then refluxed for 2 h. The reaction mixture was cooled to room temperature and the solvent evaporated. The residue was chromatographed (ethyl acetate/isohexane, 10-25%) to give 1,1-dimethylethyl 7-(5-{3-cyano-4-[(1-methylethyl)oxy]phenyl}-1,2,4-oxadiazol-3-yl)-8-methyl-1,2,4,5-tetrahydro-3H-3-benzazepine-3-carboxylate as a pale yellow oil which solidified (1.15 g).

LCMS (Method formate): Retention time 1.59 min, MH⁺ not seen

### Preparation 16

### 1,1-dimethylethyl 7-[(hydroxyamino)(imino)methyl]-8-methyl-1,2,4,5-tetrahydro-3H-3-benzazepine-3-carboxylate

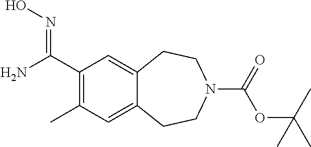

A mixture of 1,1-dimethylethyl 7-cyano-8-methyl-1,2,4,5-tetrahydro-3H-3-benzazepine-3-carboxylate (Preparation 17) (1.25 g, 4.4 mmol), hydroxylamine hydrochloride (1.52 g, 22 mmol) and sodium hydrogen carbonate (1.83 g, 22 mmol) in ethanol (20 ml) was refluxed for 24 h. The reaction mixture was cooled to room temperature and filtered through Celite™. The solvent was evaporated from the filtrate to give 1,1-dimethylethyl 7-[(hydroxyamino)(imino)methyl]-8-methyl-1,2,4,5-tetrahydro-3H-3-benzazepine-3-carboxylate as a colourless solid which was used without further purification in the subsequent reaction (Preparation 15).

LCMS (Method HpH): Retention time 0.97 min, MH⁺=320

### Preparation 17

### 1,1-dimethylethyl 7-cyano-8-methyl-1,2,4,5-tetrahydro-3H-3-benzazepine-3-carboxylate

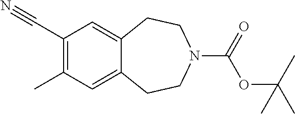

A solution of 1,1-dimethylethyl 7-methyl-8-{[(trifluoromethyl)sulfonyl]oxy}-1,2,4,5-tetrahydro-3H-3-benzazepine-3-carboxylate (Preparation 18) (2.0 g, 4.9 mmol) in DMF (40 ml) was degassed by alternately evacuating the flask and then purging with nitrogen (3 cycles) then zinc cyanide (0.75 g, 6.4 mmol) and tetrakis(triphenylphosphine) palladium (0.40 g, 0.34 mmol) were added and the mixture was heated at 120° C. for 6 h. The mixture was cooled and the volatiles evaporated in vacuo. The residue was suspended in ethyl acetate (100 ml), filtered through a Celite™ pad, the filtrate washed with water (2×100 ml), dried and evaporated to give a yellow solid. This was purified by chromatography eluting with an ethyl acetate/cyclohexane gradient (0-50%) to give 1,1-dimethylethyl 7-cyano-8-methyl-1,2,4,5-tetrahydro-3H-3-benzazepine-3-carboxylate (1.28 g, 92%) as white solid.

LCMS (Method formate): Retention time 1.26 min, MH⁺=287

### Preparation 18

### 1,1-dimethylethyl 7-methyl-8-{[(trifluoromethyl)sulfonyl]oxy}-1,2,4,5-tetrahydro-3H-3-benzazepine-3-carboxylate

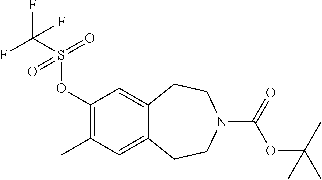

Triflic anhydride (1.346 ml, 7.97 mmol) was added dropwise to a solution of 1,1-dimethylethyl 7-hydroxy-8-methyl-1,2,4,5-tetrahydro-3H-3-benzazepine-3-carboxylate (Preparation 19) (1.7 g, 6.1 mmol) and pyridine (0.99 ml, 12 mmol) in DCM (60 ml) at −70° C. and the mixture was then allowed to warm slowly to room temperature, giving a pale yellow solution. The solution was washed with water (100 ml) and hydrochloric acid (0.5M, 100 ml), dried and evaporated to give a pale yellow oil. This was purified by chromatography eluting with an ethyl acetate/cyclohexane gradient (0-30%) to give 1,1-dimethylethyl 7-methyl-8-{[(trifluoromethyl)sulfonyl]oxy}-1,2,4,5-tetrahydro-3H-3-benzazepine-3-carboxylate (2.02 g, 80%) as colourless oil.

LCMS (Method formate): Retention time 1.47 min, [M−H]=408

### Preparation 19

### 1,1-dimethylethyl 7-hydroxy-8-methyl-1,2,4,5-tetrahydro-3H-3-benzazepine-3-carboxylate

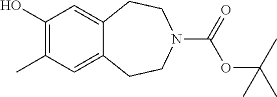

Ethyl chloroformate (1.80 ml, 19 mmol) was added to a solution of 1,1-dimethylethyl 7-hydroxy-8-(hydroxymethyl)-1,2,4,5-tetrahydro-3H-3-benzazepine-3-carboxylate (Preparation 20) (2.2 g, 7.5 mmol) and triethylamine (1.25 ml, 9.0 mmol) in THF (50 ml) at 0° C. and the solution was stirred for 2 h, then warmed to room temperature and the mixture filtered. The filtrate was evaporated, the residue redissolved in THF (15 ml) and the solution added dropwise to a solution of sodium borohydride (2.27 g, 60 mmol) in water (15 ml) at 0° C. The mixture was stirred for 1 h, neutralised with hydrochloric acid (1 M) added dropwise, diluted with water (30 ml) and extracted with ethyl acetate (2×50 ml). The solvent was dried and evaporated to give a white solid. This solid was dissolved in DCM (15 ml) and loaded onto a column, then eluted with an ethyl acetate/cyclohexane gradient (0-50%) to give 1,1-dimethylethyl 7-hydroxy-8-methyl-1,2,4,5-tetrahydro-3H-3-benzazepine-3-carboxylate (1.70 g, 82%) as white solid. This was used without purification in the subsequent reaction (Preparation 18).

LCMS (Method formate): Retention time 1.14 min, MH⁺=278

### Preparation 20

### 1,1-dimethylethyl 7-hydroxy-8-(hydroxymethyl)-1,2,4,5-tetrahydro-3H-3-benzazepine-3-carboxylate

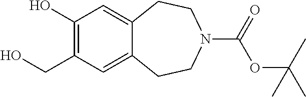

1,1-Dimethylethyl 7-hydroxy-1,2,4,5-tetrahydro-3H-3-benzazepine-3-carboxylate (5.2 g, 20 mmol, Journal of Medicinal Chemistry (2007), 50(21), 5076-5089), acetic acid (0.34 ml), paraformaldehyde (1 g) and phenylboronic acid (2.89 g, 24 mmol) were suspended in toluene (100 ml) and heated under a dean stark apparatus for 18 h at reflux. Additional paraformaldehyde (1 g) was added at 2, 4, and 8 h into the reaction.

The mixture was cooled and evaporated and the residue was partitioned between ethyl acetate (100 ml) and water (100 ml), the organic washed with water (100 ml), dried and evaporated in vacuo. The residue was dissolved in THF (60 ml) and cooled in ice, then hydrogen peroxide (35% v/v, 20 ml) was added and the solution stirred for 1 h at 0° C., then allowed to warm to room temperature over 2 h. The mixture was diluted with ethyl acetate (200 ml), washed with water (200 ml) and aqueous sodium metabisulphite solution (10%, 200 ml), dried and evaporated. The residue was suspended in DCM and the solid isolated by filtration to give 1,1-dimethylethyl 7-hydroxy-8-(hydroxymethyl)-1,2,4,5-tetrahydro-3H-3-benzazepine-3-carboxylate (2.20 g, 38%) as a white powder, which was used without further purification in the subsequent reaction (Preparation 19).

LCMS (Method formate): Retention time 0.92 min, MH⁺=294

The filtrate was loaded onto a column and purified by chromatography eluting with an ethyl acetate/cyclohexane gradient (0-50%) to give a further portion of 1,1-dimethylethyl 7-hydroxy-8-(hydroxymethyl)-1,2,4,5-tetrahydro-3H-3-benzazepine-3-carboxylate (0.60 g, 10%).

LCMS (Method formate): Retention time 0.91 min, MH⁺=294

### Preparation 21

### 1,1-Dimethylethyl 5-methyl-6-{[(trifluoromethyl)sulfonyl]oxy}-3,4-dihydro-2(1H)-isoquinolinecarboxylate

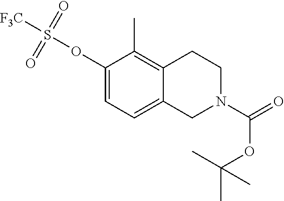

To a solution of 1,1-dimethylethyl 6-hydroxy-5-methyl-3,4-dihydro-2(1H)-isoquinolinecarboxylate (Preparation 168) (3.16 g, 12 mmol) in DCM (50 ml) at room temperature under nitrogen was added pyridine (1.94 ml, 24 mmol) and the resulting solution was cooled to −30° C. before trifluoromethanesulfonic anhydride (2.23 ml, 13.20 mmol) was added dropwise. The resulting mixture was stirred for 40 min at this temperature, warmed to room temperature and concentrated. The residue was diluted with ethyl acetate and washed sequentially with a hydrochloric acid (1N), saturated sodium hydrogen carbonate and brine. The solution was dried (MgSO₄) and concentrated in vacuo to give 1,1-dimethylethyl 5-methyl-6-{[(trifluoromethyl)sulfonyl]oxy}-3,4-dihydro-2(1H)-isoquinolinecarboxylate (4.85 g, 102%) as a red oil which was used in the next step (Preparation 22) without further purification.

LCMS (Method HpH): Retention time 1.46 min, [M−H]=394

1H NMR (CDCl₃): δ H 7.10 (1H, d), 7.02 (1H, d), 4.58 (2H, s), 3.68 (2H, t), 2.76 (2H, t), 2.25 (3H, s), 1.49 (9H, s).

### Preparation 22

### 1,1-Dimethylethyl 6-cyano-5-methyl-3,4-dihydro-2(1H)-isoquinolinecarboxylate

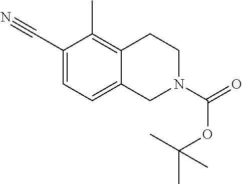

A solution of 1,1-dimethylethyl 5-methyl-6-{[(trifluoromethyl)sulfonyl]oxy}-3,4-dihydro-2(1H)-isoquinolinecarboxylate (Preparation 21) (26.11 g, 66 mmol) in DMF (200 ml) was de-gassed for 10 min under vacuum then flushed with nitrogen. The solution was treated with tetrakis(triphenylphosphine)palladium (7.63 g, 6.6 mmol) and zinc cyanide (10.08 g, 86 mmol) and the resulting mixture was stirred at 100° C. under nitrogen for 6 h and cooled to room temperature. The mixture was filtered, the residue washed with ethyl acetate and most of the solvent evaporated in vacuo. The residue was dissolved in ethyl acetate and washed with saturated sodium hydrogen carbonate (x2). The combined aqueous phases were extracted with ethyl acetate (x2) and the combined organic phases were washed with brine, dried (MgSO₄) and concentrated in vacuo. Purification of the residue by flash chromatography eluting with an ethyl acetate/cyclohexane gradient gave 1,1-dimethylethyl 6-cyano-5-methyl-3,4-dihydro-2(1H)-isoquinolinecarboxylate (16.6 g, 92%) as a white solid.

LCMS (Method HpH): Retention time 1.24 min, MH⁺=273

1H NMR (CDCl₃): δ H 7.43 (1H, d), 7.04 (1H, d), 4.60 (2H, s), 3.69 (2H, t), 2.75 (2H, t), 2.46 (3H, s), 1.49 (9H, s).

### Preparation 23

### 1,1-Dimethylethyl 6-[(hydroxyamino)(imino)methyl]-5-methyl-3,4-dihydro-2(1H)-isoquinolinecarboxylate

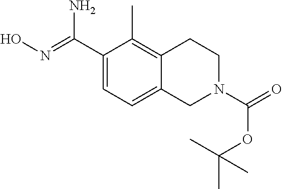

A mixture of 1,1-dimethylethyl 6-cyano-5-methyl-3,4-dihydro-2(1H)-isoquinolinecarboxylate (Preparation 22) (16.6 g, 61 mmol), sodium hydrogen carbonate (30.7 g, 370 mmol) and hydroxylamine hydrochloride (25.4 g, 370 mmol) in ethanol (250 ml) was refluxed for 28.5 h and allowed to cool to room temperature. The reaction was filtered and the residue washed with ethanol. The combined filtrate and washings were concentrated in vacuo. The residue was poured into water (100 ml) and stirred at room temperature for 20 min. The precipitated solid was isolated by filtration and dried under vacuum at 40° C. for 16 h to give 1,1-dimethylethyl 6-[(hydroxyamino)(imino)methyl]-5-methyl-3,4-dihydro-2(1H)-isoquinolinecarboxylate (16 g, 86%) as a white solid.

LCMS (Method HpH): Retention time 0.93 min, MH⁺=306

### Preparation 23: Alternative Procedure

### 1,1-dimethylethyl 6-[(hydroxyamino)(imino)methyl]-5-methyl-3,4-dihydro-2(1H)-isoquinolinecarboxylate

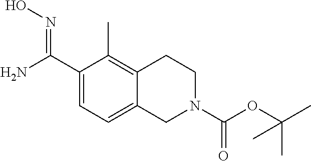

To the solution of 1,1-dimethylethyl 6-cyano-5-methyl-3,4-dihydro-2(1H)-isoquinolinecarboxylate in toluene from Preparation 170 was added toluene (10 l), the solution filtered through a Cuno Zeta Carbon filter and the filter washed with toluene (39.6 kg). The combined filtrate and washings were reduced to 27 l by vacuum distillation. Ethanol (71.9 l) was added to the cooled residue and the mixture reduced to 46 l by vacuum distillation. The residual solution was held at 35±3° C. overnight, ethanol (36.4 kg) added and the mixture reduced to 36 l by vacuum distillation. The mixture was warmed to 50±3° C., treated with hydroxylamine (50% w/w, 17.7 kg) and ethanol (7.2 kg) and heated at 75±3° C. under nitrogen for 22 h. The reaction was cooled to 60±3° C., stirred at this temperature for 30 min, further cooled to 10±3° C. and maintained at this temperature for 3 h. The mixture was filtered and the solid washed with cold (0±3° C.) ethanol (2×21.4 kg). Remaining solvent was evaporated from the solid under nitrogen pressure and the solid further dried under vacuum at 45±5° C. to give 1,1-dimethylethyl 6-[(hydroxyamino)(imino)methyl]-5-methyl-3,4-dihydro-2(1H)-isoquinolinecarboxylate (7.95 kg).

1H NMR (D₆-DMSO): δ H 9.23 (1H, s), 7.07 (1H, d), 6.99 (1H, d), 5.66 (2H, s), 4.48 (2H, s), 3.58 (2H, t), 2.67 (2H, t), 2.20 (3H, s), 1.42 (9H, s).

### Preparation 24

### 1,1-Dimethylethyl 6-(5-{3-cyano-4-[(1-methylethyl)oxy]phenyl}-1,2,4-oxadiazol-3-yl)-5-methyl-3,4-dihydro-2(1H)-isoquinolinecarboxylate

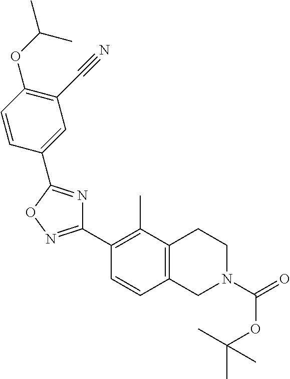

To a suspension of 1,1-dimethylethyl 6-[(hydroxyamino)(imino)methyl]-5-methyl-3,4-dihydro-2(1H)-isoquinolinecarboxylate (Preparation 23) (4.58 g, 15 mmol) in toluene (30 ml) and pyridine (30 ml) at room temperature under nitrogen was slowly added 3-cyano-4-[(1-methylethyl)oxy]benzoyl chloride (Preparation 169) (3.52 g, 16 mmol) in toluene (15 ml). After 15 min, the resulting mixture was gently refluxed for 90 min (internal temperature 110° C.) then cooled to room temperature. The solution was decanted from the brown precipitate, the precipitate washed with toluene and the combined organics concentrated in vacuo. The residue was dissolved in ethyl acetate and the resulting solution washed with hydrochloric acid (2N). The aqueous phase was extracted with ethyl acetate and the combined organic phases were washed sequentially with saturated aqueous sodium hydrogen carbonate and brine, dried (MgSO₄) and concentrated in vacuo. Purification of the residue by flash chromatography eluting with an ethyl acetate/cyclohexane, gradient gave 1,1-dimethylethyl 6-(5-{3-cyano-4-[(1-methylethyl)oxy]phenyl}-1,2,4-oxadiazol-3-yl)-5-methyl-3,4-dihydro-2(1H)-isoquinolinecarboxylate (3.62 g, 51%) as a white foam.

LCMS (Method HpH): Retention time 1.55 min, MH⁺=475 (weak)

1H NMR (CDCl₃): δ H 8.42 (1H, d), 8.33 (1H, dd), 7.76 (1H, d), 7.13-7.10 (2H, m), 4.79 (1H, m), 4.64 (2H, s), 3.72 (2H, t), 2.84 (2H, t), 2.52 (3H, s), 1.51 (9H, s), 1.48 (6H, d).

### Preparation 24: Alternative Procedure

### 1,1-dimethylethyl 6-(5-{3-cyano-4-[(1-methylethyl)oxy]phenyl}-1,2,4-oxadiazol-3-yl)-5-methyl-3,4-dihydro-2(1H)-isoquinolinecarboxylate

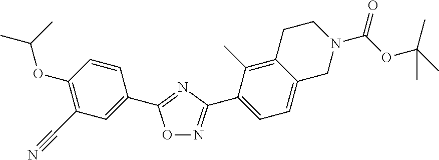

To a suspension of 3-cyano-4-isopropyloxybenzoic acid (369.6 g) in toluene (3850 ml) was added oxalyl chloride (183 ml) whilst keepinging the reaction temperature below 30° C. The reaction was heated at 55° C. for 35 min and stirred overnight. Oxalyl chloride (31 ml) was added and the reaction heated at 55° C. for ˜3 h. The reaction was cooled to 50° C., concentrated by vacuum distillation (31 distillate removed) and the reaction cooled to 20° C. The residue was added over ˜20 min to a mixture of 1,1-dimethylethyl 6-[(hydroxyamino)(imino)methyl]-5-methyl-3,4-dihydro-2(1H)-isoquinolinecarboxylate (Preparation 23) (549.9 g), anhydrous toluene (4.4 l) and anhydrous pyridine (175 ml). The acid chloride was washed in with anhydrous toluene (560 ml) and the reaction heated at 36-44° C. for 1 h. The reaction was heated to 80° C. over ˜30 min and maintained at this temperature for 13.5 h. The reaction was slowly cooled to 20° C. (over ˜1 h), ethyl acetate (3.3 l) and sodium carbonate solution (5% w/w, 5.5 l) added. The mixture was stirred for ˜5 min, the aqueous discarded and the organic washed with water (5.5 l). The organic phase was filtered (5μ Dominick Hunter filter) and reduced to dryness in vacuo to give 1,1-dimethylethyl 6-(5-{3-cyano-4-[(1-methylethyl)oxy]phenyl}-1,2,4-oxadiazol-3-yl)-5-methyl-3,4-dihydro-2(1H)-isoquinolinecarboxylate (757 g, 83%).

1H NMR (CDCl₃): δ H 8.42 (1H, d), 8.33 (1H, dd), 7.76 (1H, d), 7.13-7.10 (2H, m), 4.80 (1H, m), 4.64 (2H, s), 3.72 (2H, t), 2.52 (2H, t), 1.51 (9H, s), 1.48 (6H, d).

### Preparation 25

### 2-[(1-Methylethyl)oxy]-5-[3-(5-methyl-1,2,3,4-tetrahydro-6-isoquinolinyl)-1,2,4-oxadiazol-5-yl]benzonitrile hydrochloride

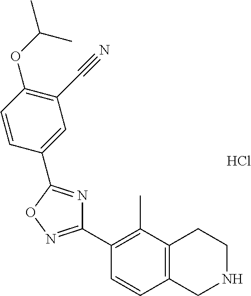

To a solution of 1,1-dimethylethyl 6-(5-{3-cyano-4-[(1-methylethyl)oxy]phenyl}-1,2,4-oxadiazol-3-yl)-5-methyl-3,4-dihydro-2(1H)-isoquinolinecarboxylate (Preparation 24) (3.4 g, 7.2 mmol) in 1,4-dioxane (20 ml) at room temperature under nitrogen was added a hydrogen chloride in 1,4-dioxane (4M, 17.9 ml, 72 mmol) and the resulting mixture was stirred at this temperature for 5.5 h, stored in a freezer overnight and then concentrated. The residue was co-evaporated with diethyl ether to give 2-[(1-methylethyl)oxy]-5-[3-(5-methyl-1,2,3,4-tetrahydro-6-isoquinolinyl)-1,2,4-oxadiazol-5-yl]benzonitrile hydrochloride (2.88 g, 98%) as a white solid.

LCMS (Method HpH): Retention time 1.21 min, MH⁺=375

1H NMR (D₆-DMSO): δ H 9.54 (2H, bs), 8.50, (1H, d), 8.40 (1H, dd), 7.77 (1H, d), 7.56 (1H, d), 7.29 (1H, d), 4.98 (1H, m), 4.35 (2H, s), 3.45 (2H, t), 3.00 (2H, t), 2.47 (3H, s), 1.39 (6H, d).

### Preparation 25: Alternative Procedure

### 2-[(1-Methylethyl)oxy]-5-[3-(5-methyl-1,2,3,4-tetrahydro-6-isoquinolinyl)-1,2,4-oxadiazol-5-yl]benzonitrile trifluoroacetate

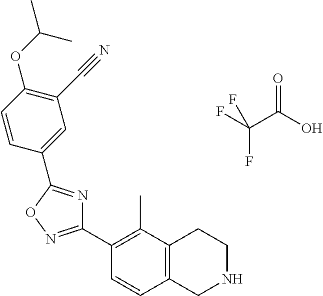

Trifluoroacetic acid (15 ml) was added to an ice cooled solution of 1,1-dimethylethyl 6-(5-{3-cyano-4-[(1-methylethyl)oxy]phenyl}-1,2,4-oxadiazol-3-yl)-5-methyl-3,4-dihydro-2(1H)-isoquinolinecarboxylate (Preparation 24) (2.9 g, 6.1 mmol) in DCM (20 ml). The reaction mixture was stirred at 0° C. for 1 h and the solvent evaporated. The residue was co-evaporated with toluene (x2) and triturated with diethyl ether. The solid was isolated by filtration and washed with diethyl ether to give 2-[(1-methylethyl)oxy]-5-[3-(5-methyl-1,2,3,4-tetrahydro-6-isoquinolinyl)-1,2,4-oxadiazol-5-yl]benzonitrile trifluoroacetate (2.7 g, 90%) as a colourless solid.

LCMS (Method formate): Retention time 0.90 min, MH⁺=375

1H NMR (D₆-DMSO): δ H 9.16 (2H, bs), 8.51, (1H, d), 8.40 (1H, dd), 7.78 (1H, d), 7.57 (1H, d), 7.29 (1H, d), 4.98 (1H, m), 4.38 (2H, s), 3.49 (2H, partially obscured by water), 2.99 (2H, t), 2.47 (3H, s), 1.39 (6H, d).

### Preparation 25: Alternative Procedure

### 2-[(1-methylethyl)oxy]-5-[3-(5-methyl-1,2,3,4-tetrahydro-6-isoquinolinyl)-1,2,4-oxadiazol-5-yl]benzonitrile hydrochloride

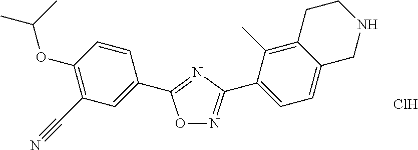

1,1-Dimethylethyl 6-(5-{3-cyano-4-[(1-methylethyl)oxy]phenyl}-1,2,4-oxadiazol-3-yl)-5-methyl-3,4-dihydro-2(1H)-isoquinolinecarboxylate (Preparation 24) (50.0 g, 110 mmol) in DCM (150 ml) was added drop-wise to hydrogen chloride in 1,4-dioxane (4M, 263 ml, 1100 mmol) and the mixture was stirred for 2 h at room temperature, giving a pale yellow suspension. The mixture was diluted with diethyl ether (500 ml), stirred for 20 min. Then solid was isolated by filtration, washed with diethyl ether (3×100 ml) and dried in vacuo at 55° C. overnight to give 2-[(1-methylethyl)oxy]-5-[3-(5-methyl-1,2,3,4-tetrahydro-6-isoquinolinyl)-1,2,4-oxadiazol-5-yl]benzonitrile hydrochloride (39.8 g, 92%) as white solid.

LCMS (Method HpH): Retention time 1.22 min, MH⁺=375

1H NMR (D₆-DMSO) includes: δ H 9.49 (2H, bs), 8.51 (1H, d), 8.40 (1H, dd), 7.77 (1H, d), 7.56 (1H, d), 7.29 (1H, d), 4.98 (1H, m), 4.35 (2H, m), 3.44-3.36 (2H, largely obscured by water), 3.00 (2H, t), 2.47 (3H, s), 1.39 (6H, d).

### Preparation 26

### Ethyl 3-[6-(5-{3-cyano-4-[(1-methylethyl)oxy]phenyl}-1,2,4-oxadiazol-3-yl)-5-methyl-3,4-dihydro-2(1H)-isoquinolinyl]propanoate

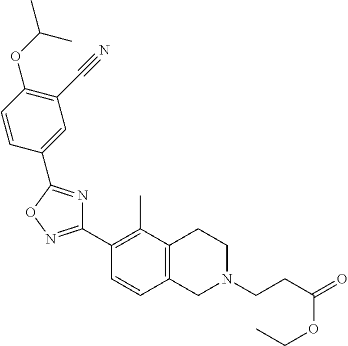

A mixture of 2-[(1-methylethyl)oxy]-5-[3-(5-methyl-1,2,3,4-tetrahydro-6-isoquinolinyl)-1,2,4-oxadiazol-5-yl]benzonitrile trifluoroacetic acid salt (Preparation 25) (220 mg, 0.45 mmol), ethyl acrylate (74 μl, 0.68 mmol) and DBU (204 μl, 1.2 mmol) in acetonitrile (5 ml) was stirred at room temperature for 3 h and the solvent evaporated. The residue was dissolved in ethyl acetate (10 ml) and the solution washed with water (3×5 ml), dried and concentrated. Purification of the residue by chromatography (ethyl acetate/iso-hexane, 30%) gave ethyl 3-[6-(5-{3-cyano-4-[(1-methylethyl)oxy]phenyl}-1,2,4-oxadiazol-3-yl)-5-methyl-3,4-dihydro-2(1H)-isoquinolinyl]propanoate (209 mg, 98%) as a colourless oil.

LCMS (Method formate): Retention time 0.98 min, MH⁺=475

### Preparation 27

### 3-[6-(5-{3-Cyano-4-[(1-methylethyl)oxy]phenyl}-1,2,4-oxadiazol-3-yl)-5-methyl-3,4-dihydro-2(1H)-isoquinolinyl]propanoic acid sodium salt

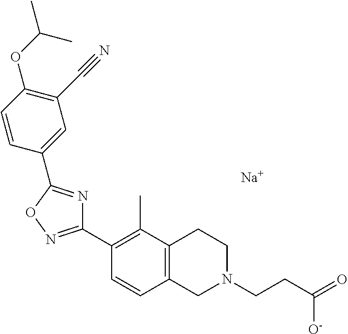

Sodium hydroxide (2M, 1 ml) was added to a stirred solution of ethyl 3-[6-(5-{3-cyano-4-[(1-methylethyl)oxy]phenyl}-1,2,4-oxadiazol-3-yl)-5-methyl-3,4-dihydro-2(1H)-isoquinolinyl]propanoate (Preparation 26) (200 mg, 0.42 mmol) in ethanol (1 ml). The reaction mixture was stirred at 50° C. for 1 h then cooled and the ethanol evaporated. The residue was diluted with water (2 ml) and stirred for 15 min. The precipitate was isolated by filtration, washed with water and dried under vacuum to give 3-[6-(5-{3-cyano-4-[(1-methylethyl)oxy]phenyl}-1,2,4-oxadiazol-3-yl)-5-methyl-3,4-dihydro-2(1H)-isoquinolinyl]propanoic acid sodium salt (150 mg, 76%) as a colourless solid.

LCMS (Method formate): Retention time 0.92 min, MH⁺=447

### Preparation 28

### Ethyl [6-(5-{3-cyano-4-[(1-methylethyl)oxy]phenyl}-1,2,4-oxadiazol-3-yl)-5-methyl-3,4-dihydro-2(1H)-isoquinolinyl]acetate

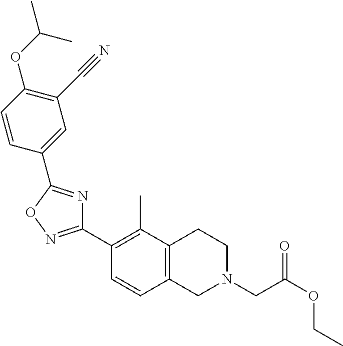

Ethyl bromoacetate (188 mg, 1.1 mmol) was added to a suspension of 2-[(1-methylethyl)oxy]-5-[3-(5-methyl-1,2,3,4-tetrahydro-6-isoquinolinyl)-1,2,4-oxadiazol-5-yl]benzonitrile trifluoroacetic salt (Preparation 25) (500 mg, 1.0 mmol) and potassium carbonate (311 mg, 2.3 mmol) in DMF (5 ml) and the mixture was stirred at 80° C. for 1 h, cooled and added to water (50 ml). The mixture was extracted with ethyl acetate (50 ml), the organic phase washed with water (50 ml), dried and concentrated to give ethyl [6-(5-{3-cyano-4-[(1-methylethyl)oxy]phenyl}-1,2,4-oxadiazol-3-yl)-5-methyl-3,4-dihydro-2(1H)-isoquinolinyl]acetate (0.405 g, 86%) as a colourless gum which was used in the next step (Preparation 29) without further purification.

LCMS (Method formate): Retention time 1.02 min, MH⁺=461

### Preparation 29

### [6-(5-{3-Cyano-4-[(1-methylethyl)oxy]phenyl}-1,2,4-oxadiazol-3-yl)-5-methyl-3,4-dihydro-2(1H)-isoquinolinyl]acetic acid sodium salt

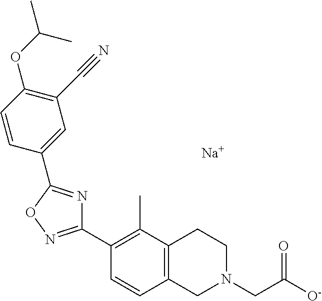

Sodium hydroxide (2M, 0.869 ml, 1.7 mmol) was added to a suspension of ethyl [6-(5-{3-cyano-4-[(1-methylethyl)oxy]phenyl}-1,2,4-oxadiazol-3-yl)-5-methyl-3,4-dihydro-2(1H)-isoquinolinyl]acetate (Preparation 28) (400 mg, 0.87 mmol) in ethanol (20 ml) and the mixture stirred for 2 h at room temperature. The suspension was filtered, the isolated solid washed with ethanol (2×10 ml), and dried at 50° C. for 3 h to give [6-(5-{3-cyano-4-[(1-methylethyl)oxy]phenyl}-1,2,4-oxadiazol-3-yl)-5-methyl-3,4-dihydro-2(1H)-isoquinolinyl]acetic acid sodium salt (286 mg, 72%) as a white solid.

LCMS (Method formate): Retention time 0.90 min, MH⁺=433

### Preparation 29: Alternative Procedure [6-(5-{3-cyano-4-[(1-methylethyl)oxy]phenyl}-1,2,4-oxadiazol-3-yl)-5-methyl-3,4-dihydro-2(1H)-isoquinolinyl]acetic acid

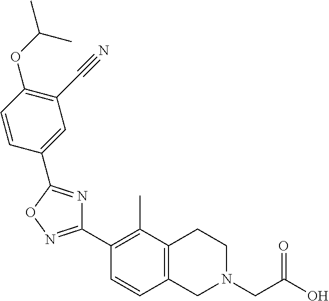

A mixture of methyl [6-(5-{3-cyano-4-[(1-methylethyl)oxy]phenyl}-1,2,4-oxadiazol-3-yl)-5-methyl-3,4-dihydro-2(1H)-isoquinolinyl]acetate (Preparation 189) (1.6 g, 3.6 mmol), ethanol (20 ml) and sodium hydroxide (2M, 20 ml) was stirred at room temperature overnight. The ethanol was evaporated. The residue was diluted with water (20 ml) and the mixture was acidified with glacial acetic acid. The mixture was stirred for 20 min. The solid was isolated by filtration and washed with water. The solid was dried to give [6-(5-{3-cyano-4-[(1-methylethyl)oxy]phenyl}-1,2,4-oxadiazol-3-yl)-5-methyl-3,4-dihydro-2(1H)-isoquinolinyl]acetic acid (1.4 g) as a colourless solid.

LCMS (Method formate): Retention time 0.91 min, MH⁺=433

### Preparation 30

Ethyl 4-[6-(5-{3-cyano-4-[(1-methylethyl)oxy]phenyl}-1,2,4-oxadiazol-3-yl)-5-methyl-3,4-dihydro-2(1H)-isoquinolinyl]butanoate

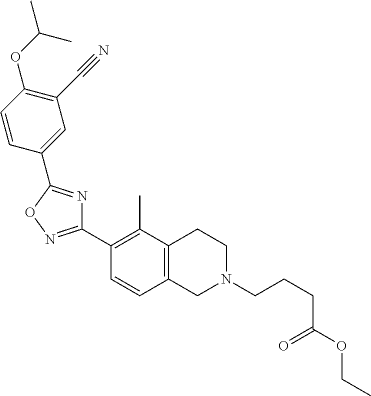

Potassium carbonate (120 mg, 0.95 mmol) was added to a stirred solution of 2-[(1-methylethyl)oxy]-5-[3-(5-methyl-1,2,3,4-tetrahydro-6-isoquinolinyl)-1,2,4-oxadiazol-5-yl]benzonitrile trifluoroacetic salt (Preparation 25) (210 mg, 0.43 mmol) and ethyl 4-bromobutyrate (68 μl, 0.47 mmol) in dry DMF (5 ml). The reaction mixture was stirred at 80° C. for 6 h and diluted with ethyl acetate (25 ml). The mixture was washed with water (x2), brine, dried and concentrated. Purification of the residue by chromatography (ethyl acetate/iso-hexane, 20-40%) gave ethyl 4-[6-(5-{3-cyano-4-[(1-methylethyl)oxy]phenyl}-1,2,4-oxadiazol-3-yl)-5-methyl-3,4-dihydro-2(1H)-isoquinolinyl}butanoate (190 mg, 90%) as a yellow oil which was used without further purification.

LCMS (Method formate): Retention time 1.01 min, MH⁺=489

### Preparation 31

### 4-[6-(5-{3-Cyano-4-[(1-methylethyl)oxy]phenyl}-1,2,4-oxadiazol-3-yl)-5-methyl-3,4-dihydro-2(1H)-isoquinolinyl]butanoic acid sodium salt

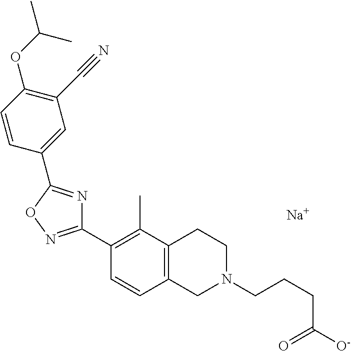

Sodium hydroxide (2M, 1 ml) was added to a stirred solution of ethyl 4-[6-(5-{3-cyano-4-[(1-methylethyl)oxy]phenyl}-1,2,4-oxadiazol-3-yl)-5-methyl-3,4-dihydro-2(1H)-isoquinolinyl]butanoate (Preparation 30) (190 mg, 0.39 mmol) in ethanol (1 ml). The reaction mixture was stirred at 50° C. for 1 h, cooled and the ethanol evaporated. The residue was diluted with water (2 ml) and stirred for 15 min. The precipitate was isolated by filtration, washed with water and dried under vacuum to give 4-[6-(5-{3-cyano-4-[(1-methylethyl)oxy]phenyl}-1,2,4-oxadiazol-3-yl)-5-methyl-3,4-dihydro-2(1H)-isoquinolinyl]butanoic acid sodium salt (140 mg, 74%) as a colourless solid.

LCMS (Method formate): Retention time 0.91 min, MH⁺=461

### Preparation 32

### 1,1-Dimethylethyl 5-methyl-6-(4,4,5,5-tetramethyl-1,3,2-dioxaborolan-2-yl)-3,4-dihydro-2(1H)-isoquinolinecarboxylate

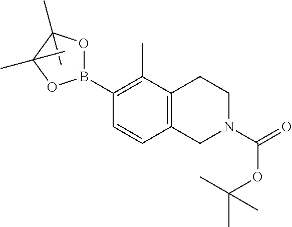

A solution of 1,1-dimethylethyl 5-methyl-6-{[(trifluoromethyl)sulfonyl]oxy}-3,4-dihydro-2(1H)-isoquinolinecarboxylate (Preparation 21) (3.94 g, 10 mmol, WO2009080724) in 1,4-dioxane (40 ml) was de-gassed under vacuum for 10 min. 1,1′-Bis(diphenylphosphino)ferrocenedichloro palladium (II) (0.73 g, 1.0 mmol), potassium acetate (3.9 g, 40 mmol) and 4,4,4′,4′,5,5,5′,5′-octamethyl-2,2′-bi-1,3,2-dioxaborolane (3.0 g, 12 mmol) were added and the resulting mixture was refluxed. The reaction was concentrated in vacuo, the residue diluted with ethyl acetate and water and the biphasic mixture was filtered to remove insoluble palladium residues. The two phases were separated and the aqueous phase extracted with ethyl acetate. The combined organic phases were washed with brine, dried (MgSO₄) and concentrated in vacuo. Purification of the residue by flash chromatography (ethyl acetate/cyclohexane, 3-10%) gave 1,1-dimethylethyl 5-methyl-6-(4,4,5,5-tetramethyl-1,3,2-dioxaborolan-2-yl)-3,4-dihydro-2(1H)-isoquinolinecarboxylate (3.41 g, 92%) as a white solid.

LCMS (Method formate): Retention time 1.52 min, [2M+H]⁺=747

### Preparation 33

### 1,1-Dimethylethyl 6-(5-{3-cyano-4-[(1-methylethyl)oxy]phenyl}-1,3,4-thiadiazol-2-yl)-5-methyl-3,4-dihydro-2(1H)-isoquinolinecarboxylate

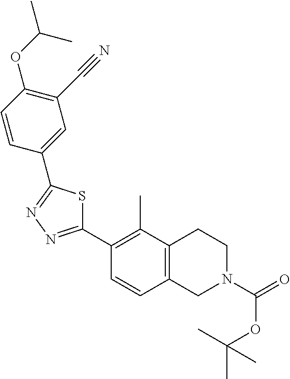

A mixture of 1,1-dimethylethyl 5-methyl-6-(4,4,5,5-tetramethyl-1,3,2-dioxaborolan-2-yl)-3,4-dihydro-2(1H)-isoquinolinecarboxylate (Preparation 32) (2.13 g, 5.7 mmol), sodium carbonate (3.02 g, 29 mmol), 5-(5-bromo-1,3,4-thiadiazol-2-yl)-2-[(1-methylethyl)oxy]benzonitrile (Preparation 2) (2.22 g, 6.9 mmol) and 1,1′-bis(diphenylphosphino)ferrocenedichloro palladium(II) dichloromethane complex (0.42 g, 0.57 mmol) in DME (30 ml) and water (10 ml) was refluxed under nitrogen for 4 h. A further portion of 1,1′-bis(diphenylphosphino)ferrocenedichloro palladium(II) dichloromethane complex (200 mg) was added and the mixture refluxed for a further 2 h. The reaction was cooled to room temperature, most of the DME removed in vacuo and the residue was partitioned between ethyl acetate and water. The aqueous phase was extracted with ethyl acetate, the combined organic phases washed with brine, dried (MgSO₄) and concentrated in vacuo. Purification of the residue by flash chromatography on silica (ethyl acetate/cyclohexane, 5-30%-) gave 1,1-dimethylethyl 6-(5-{3-cyano-4-[(1-methylethyl)oxy]phenyl}-1,3,4-thiadiazol-2-yl)-5-methyl-3,4-dihydro-2(1H)-isoquinolinecarboxylate (1.78 g, 64%) as a pale yellow foam.

LCMS (method HpH): Retention time 1.49 min, MH⁺=491

### Preparation 34

### 2-[(1-Methylethyl)oxy]-5-[5-(5-methyl-1,2,3,4-tetrahydro-6-isoquinolinyl)-1,3,4-thiadiazol-2-yl]benzonitrile hydrochloride

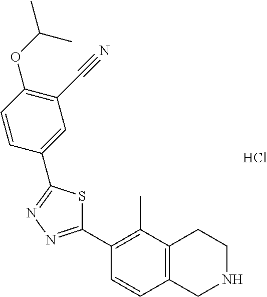

To a solution of 1,1-dimethylethyl 6-(5-{3-cyano-4-[(1-methylethyl)oxy]phenyl}-1,3,4-thiadiazol-2-yl)-5-methyl-3,4-dihydro-2(1H)-isoquinolinecarboxylate (Preparation 33) (1.78 g, 3.6 mmol) in 1,4-dioxane (16 ml) at 25° C. under nitrogen was added hydrogen chloride 1,4-dioxane (4N, 16 ml) dropwise. The resulting mixture was stirred for 3 h 20 min, concentrated in vacuo and the residue triturated with diethyl ether. Isolation of the solid by filtration gave 2-[(1-methylethyl)oxy]-5-[3-(5-methyl-1,2,3,4-tetrahydro-6-isoquinolinyl)-1,3,4-thiadiazol-2-yl]benzonitrile hydrochloride (1.48 g, 96%) as a pale yellow solid which was used without further purification.

LCMS (Method HpH): Retention time 1.15 min, MH⁺=391

### Preparation 34: Alternative Procedure

### 2-[(1-methylethyl)oxy]-5-[5-(5-methyl-1,2,3,4-tetrahydro-6-isoquinolinyl)-1,3,4-thiadiazol-2-yl]benzonitrile trifluoroacetate

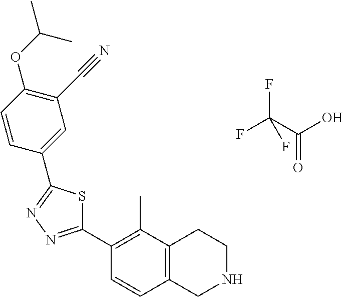

To a solution of 1,1-dimethylethyl 6-(5-{3-cyano-4-[(1-methylethyl)oxy]phenyl}-1,3,4-thiadiazol-2-yl)-5-methyl-3,4-dihydro-2(1H)-isoquinolinecarboxylate (Preparation 33) (912 mg, 1.9 mmol) in DCM (10 ml) was added trifluoroacetic acid (5.0 ml, 65 mmol) at room temperature. The resulting mixture was stirred at room temperature for 30 min, concentrated under reduced pressure and the residue obtained suspended in toluene and concentrated in vacuo (x2). The resulting brown viscous oil was triturated with diethyl ether, and the precipitate was isolated by filtration to give 2-[(1-methylethyl)oxy]-5-[5-(5-methyl-1,2,3,4-tetrahydro-6-isoquinolinyl)-1,3,4-thiadiazol-2-yl]benzonitrile trifluoroacetate (808 mg) as a brown solid.

LCMS (Method formate): Retention time 0.82 min, MH⁺=391

### Preparation 35

### Ethyl 3-[6-(5-{3-cyano-4-[(1-methylethyl)oxy]phenyl}-1,3,4-thiadiazol-2-yl)-5-methyl-3,4-dihydro-2(1H)-isoquinolinyl]propanoate

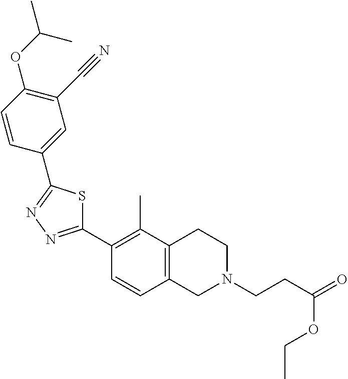

A mixture of 2-[(1-methylethyl)oxy]-5-[5-(5-methyl-1,2,3,4-tetrahydro-6-isoquinolinyl)-1,3,4-thiadiazol-2-yl]benzonitrile hydrochloride (Preparation 34) (0.85 g, 2 mmol), ethyl acrylate (261 μl, 2.4 mmol) and DBU (904 μl, 6 mmol) in acetonitrile (10 ml) was stirred at room temperature for 4 h. The mixture was diluted with ethyl acetate (20 ml), the resulting solution washed with water (x2), then brine and dried the solvent was evaporated and the residue triturated with diethyl ether to give ethyl 3-[6-(5-{3-cyano-4-[(1-methylethyl)oxy]phenyl}-1,3,4-thiadiazol-2-yl)-5-methyl-3,4-dihydro-2(1H)-isoquinolinyl]propanoate (893 mg, 91%) as a light brown solid.

LCMS (Method formate): Retention time 0.96 min, MH⁺=491

### Preparation 36

### 3-[6-(5-{3-Cyano-4-[(1-methylethyl)oxy]phenyl}-1,3,4-thiadiazol-2-yl)-5-methyl-3,4-dihydro-2(1H)-isoquinolinyl]propanoic acid Sodium salt

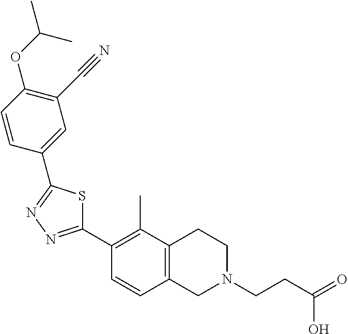

A suspension of ethyl 3-[6-(5-{3-cyano-4-[(1-methylethyl)oxy]phenyl}-1,3,4-thiadiazol-2-yl)-5-methyl-3,4-dihydro-2(1H)-isoquinolinyl]propanoate (Preparation 35) (0.88 g, 1.8 mmol) in ethanol (10 ml) was treated with a sodium hydroxide (2M, 10 ml). The reaction mixture was stirred at 50° C. for 2 h, cooled and the ethanol evaporated. The solid was isolated by filtration, washed with water and dried to give 3-[6-(5-{3-cyano-4-[(1-methylethyl)oxy]phenyl}-1,3,4-thiadiazol-2-yl)-5-methyl-3,4-dihydro-2(1H)-isoquinolinyl]propanoic acid sodium salt (790 mg, 91%) as a light brown solid.

LCMS (Method formate): Retention time 0.86 min, MH⁺=463

### Preparation 37

### Ethyl 4-[6-(5-{3-cyano-4-[(1-methylethyl)oxy]phenyl}-1,3,4-thiadiazol-2-yl)-5-methyl-3,4-dihydro-2(1H)-isoquinolinyl]butanoate

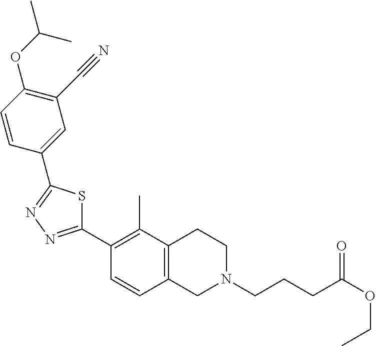

A mixture of 2-[(1-methylethyl)oxy]-5-[5-(5-methyl-1,2,3,4-tetrahydro-6-isoquinolinyl)-1,3,4-thiadiazol-2-yl]benzonitrile hydrochloride (Preparation 34) (0.85 g, 2 mmol), potassium carbonate (0.83 g, 6 mmol) and ethyl 4-bromobutyrate (470 mg, 2.4 mmol) in dry DMF (5 ml) was stirred a 70° C. for 8 h then cooled to room temperature and diluted with ethyl acetate (20 ml). The solution was washed with water (2×20 ml), dried and concentrated. Purification of the residue by chromatography eluting with DCM gave ethyl 4-[6-(5-{3-cyano-4-[(1-methylethyl)oxy]phenyl}-1,3,4-thiadiazol-2-yl)-5-methyl-3,4-dihydro-2(1H)-isoquinolinyl]butanoate (650 mg, 64%) as a brown oil which solidified on standing.

LCMS (Method formate): Retention time 0.93 min, MH⁺=505

### Preparation 38

### 4-[6-(5-{3-Cyano-4-[(1-methylethyl)oxy]phenyl}-1,3,4-thiadiazol-2-yl)-5-methyl-3,4-dihydro-2(1H)-isoquinolinyl]butanoic acid sodium salt

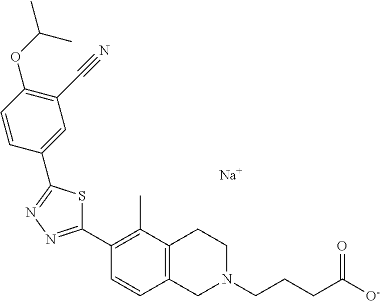

A solution of ethyl 4-[6-(5-{3-cyano-4-[(1-methylethyl)oxy]phenyl}-1,3,4-thiadiazol-2-yl)-5-methyl-3,4-dihydro-2(1H)-isoquinolinyl]butanoate (Preparation 37) (600 mg, 1.2 mmol) in ethanol (3 ml) and sodium hydroxide solution (2M, 5 ml) was stirred at 50° C. for 1 h then at room temperature for 1 h. The precipitate was isolated by filtration, washed with water and dried to give 4-[6-(5-{3-cyano-4-[(1-methylethyl)oxy]phenyl}-1,3,4-thiadiazol-2-yl)-5-methyl-3,4-dihydro-2(1H)-isoquinolinyl]butanoic acid sodium salt (438 mg, 74%) as a light brown solid.

LCMS (Method formate): Retention time 0.84 min, MH⁺=477

### Preparation 39

### 1,1-Dimethylethyl 7-(4,4,5,5-tetramethyl-1,3,2-dioxaborolan-2-yl)-1,2,4,5-tetrahydro-3H-3-benzazepine-3-carboxylate

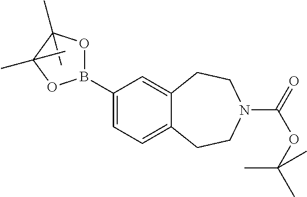

1,1-Dimethylethyl 7-{[(trifluoromethyl)sulfonyl]oxy}-1,2,4,5-tetrahydro-3H-3-benzazepine-3-carboxylate (22 g, 56 mmol, WO2002040471) and 4,4,4′,4′,5,5,5′,5′-octamethyl-2,2′-bi-1,3,2-dioxaborolane (15.6 g, 61 mmol) were dissolved in 1,4-dioxane (250 ml) and the mixture was de-gassed with nitrogen for 15 min. 1,1′-Bis(diphenylphosphino)ferrocenedichloro palladium(II) (2.92 g, 3.3 mmol) and 1,1′-bis(diphenylphosphino)ferrocene (1.85 g, 3.3 mmol) were added and the resulting mixture was stirred for 10 min under nitrogen before potassium acetate (16.39 g, 170 mmol) was added. The resulting mixture was heated at 80° C. for 3 h, allowed to cool to room temperature and diluted with ethyl acetate, water and brine. The torganic phase dried (Na₂SO₄) and concentrated in vacuum. Purification of the residue by chromatography (ethyl acetate/hexane, 10%) gave 1,1-dimethylethyl 7-(4,4,5,5-tetramethyl-1,3,2-d ioxaborolan-2-yl)-1,2,4,5-tetrahydro-3H-3-benzazepine-3-carboxylate (4.5 g, 22%) as a white solid.

### Preparation 40

### 1,1-Dimethylethyl 7-cyano-1,2,4,5-tetrahydro-3H-3-benzazepine-3-carboxylate

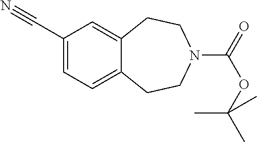

A mixture of 1,1-dimethylethyl 7-{[(trifluoromethyl)sulfonyl]oxy}-1,2,4,5-tetrahydro-3H-3-benzazepine-3-carboxylate (3.5 g, 8.9 mmol, WO2002040471) and zinc cyanide (1.25 g, 11 mmol) in dry DMF (100 ml) at room temperature was de-gassed and flushed with nitrogen several times. Tetrakis(triphenylphosphine)palladium(0) (1.02 g, 0.89 mmol) was added and the procedure repeated. The reaction mixture was then stirred under nitrogen at 100° C. for 75 min, allowed to cool to room temperature and diluted with ethyl acetate (˜100 ml). The solution was filtered through Celite™ and the Celite™ rinsed with ethyl acetate. The filtrate and washings were concentrated and the residue was partitioned between ethyl acetate and water. The mixture was re-filtered, the organic phase washed with ethyl acetate, brine, dried and concentrated. The residue was dissolved in cyclohexane, was filtered through a pad of silica, and the silica pad washed in turn with ethyl acetate/cyclohexane (100 ml of each of 10, 20 and 30%). Fractions containing product were combined and concentrated to give 1,1-dimethylethyl 7-cyano-1,2,4,5-tetrahydro-3H-3-benzazepine-3-carboxylate as a slightly orange gum which slowly crystallized on standing (2.42 g, 95%)

LCMS (Method formate): Retention time 1.22 min, MH⁺=273 weak

### Preparation 40: Alternative Procedure

### 1,1-Dimethylethyl 7-cyano-1,2,4,5-tetrahydro-3H-3-benzazepine-3-carboxylate

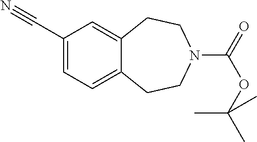

Anhydrous DMF (1.51, degassed with nitrogen for 1.5 h) was added to 1,1-dimethylethyl 7-{[(trifluoromethyl)sulfonyl]oxy}-1,2,4,5-tetrahydro-3H-3-benzazepine-3-carboxylate (Preparation 39) (130 g, Wheaton Science Products) under a blanket of nitrogen with stirring to give a solution. The whole reaction was purged under low vacuum and nitrogen for 5-10 min. The reaction was kept under a low vacuum atmosphere and free flowing zinc cyanide (50.2 g) was added rapidly, followed by rapid addition of tetrakis(triphenylphosphine) palladium (38 g). The solution was heated to 105° C. for 2 h. The reaction was cooled to room temperature, diluted with ethyl acetate (1 l) and filtered through Celite™. The solvent was evaporated until solid was precipitating out (volume of DMF remaining was ˜1 l). The reaction mixture was washed with brine and ethyl acetate (1 l), The organic phase was retained, the aqueous layer was filtered through Celite™ and the Celite™ washed with ethyl acetate (400 ml).

The filtrate and washings were partitioned and organic phases washed with brine (6×500 ml). The organic phase was dried (Na₂SO₄) and evaporated to give a dark residue. This was partially dissolved in DCM (100 m) filtered through Celite™ and cotton wool and the filtrate was loaded onto column (1500 g). The column was eluted with an ethyl acetate/cyclohexane gradient (0-100%). The first set of product fractions (light yellow) were combined and evaporated to give 1,1-dimethylethyl 7-cyano-1,2,4,5-tetrahydro-3H-3-benzazepine-3-carboxylate as an oil (76.5 g) and the second set of product fractions (slightly purple) were combined and evaporated to give 1,1-dimethylethyl 7-cyano-1,2,4,5-tetrahydro-3H-3-benzazepine-3-carboxylate (13.0 g)

LCMS (Method formate): Retention time 1.20 min, MH⁺=273

### Preparation 41

### 1,1-Dimethylethyl 7-[(hydroxyamino)(imino)methyl]-1,2,4,5-tetrahydro-3H-3-benzazepine-3-carboxylate

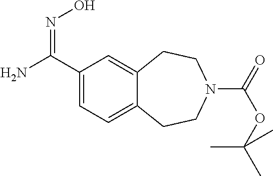

1,1-Dimethylethyl 7-cyano-1,2,4,5-tetrahydro-3H-3-benzazepine-3-carboxylate (Preparation 40) (29.4 g) was suspended in ethanol (400 ml) with stirring. Hydroxylamine hydrochloride (33.4 g) was added fairly rapidly followed by the portionwise addition of sodium hydrogen carbonate (60.5 g). When bubbling stopped the nitrogen was turned on and the reaction was heated to 60° C. and refluxed for 4 h. The reaction was concentrated to 100 ml and water (900 ml) added. Trituration and stirring gave a grey solid which was isolated by filtration and dried in vacuo overnight to give 1,1-dimethylethyl 7-[(hydroxyamino)(imino)methyl]-1,2,4,5-tetrahydro-3H-3-benzazepine-3-carboxylate (29.6 g).

LCMS (Method formate): Retention time 0.72 min, MH⁺=306

### Preparation 42

### 1,1-Dimethylethyl 7-(5-{3-cyano-4-[(1-methylethyl)oxy]phenyl}-1,2,4-oxadiazol-3-yl)-1,2,4,5-tetrahydro-3H-3-benzazepine-3-carboxylate

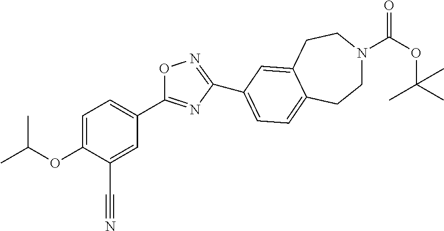

To a suspension of 1,1-dimethylethyl 7-[(hydroxyamino)(imino)methyl]-1,2,4,5-tetrahydro-3H-3-benzazepine-3-carboxylate (Preparation 41) (4.58 g, 15 mmol) in toluene (30 ml) and pyridine (30 ml) was slowly added under nitrogen at room temperature 3-cyano-4-[(1-methylethyl)oxy]benzoyl chloride (Preparation 169) (3.52 g, 16 mmol) in toluene (15 ml). After 15 min, the resulting mixture was stirred at a gentle reflux (110° C., bath at 125° C.) for 90 min then cooled to room temperature. The solvent was decanted from the brown precipitate and the precipitate washed with toluene and the combined solutions concentrated in vacuo. The residue was dissolved in ethyl acetate and washed with hydrochloric acid (2N). The aqueous phase was extracted with ethyl acetate and the combined organic phases were washed with saturated aqueous sodium hydrogen carbonate and brine, dried (MgSO₄) and concentrated in vacuo. Purification of the residue by flash chromatography (ethyl acetate/cyclohexane) gave 1,1-dimethylethyl 7-(5-{3-cyano-4-[(1-methylethyl)oxy]phenyl}-1,2,4-oxadiazol-3-yl)-1,2,4,5-tetrahydro-3H-3-benzazepine-3-carboxylate (5.53 g, 78%) as a white foam.

LCMS (Method HpH): Retention time 1.56 min, no mass ion detected.

### Preparation 42: Alternative Procedure 1,1-Dimethylethyl 7-(5-{3-cyano-4-[(1-methylethyl)oxy]phenyl}-1,2,4-oxadiazol-3-yl)-1,2,4,5-tetrahydro-3H-3-benzazepine-3-carboxylate

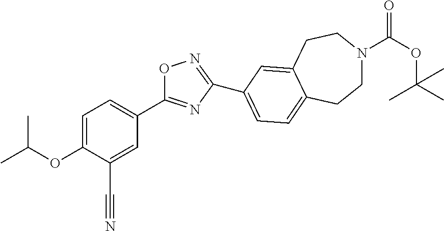

A mixture of 1,1-dimethylethyl 7-[(hydroxyamino)(imino)methyl]-1,2,4,5-tetrahydro-3H-3-benzazepine-3-carboxylate (Preparation 41) (64 g, 210 mmol) and triethylamine (35.2 ml, 250 mmol) in dry DMF (600 ml) was cooled to −5° C. 3-Cyano-4-[(1-methylethyl)oxy]benzoyl chloride (51.6 g, WO2009080730/WO2008128951) in dry DMF (163 ml) was added portionwise. After the addition the reaction was kept at 0° C. for 30 min and then allowed to rise to room temperature and maintained at this temperature for 1 h. The reaction was heated to 120° C. for 1.5 h. The reaction was cooled to room temperature and diluted with water (2 l); a precipitate was formed which was extracted into ethyl acetate (2×500 ml). The combined organic extracts were washed with water (5×500 ml), the organic phase dried (Na₂SO₄) and evaporated. The crude product was dissolved in DCM (200 ml) with heating and loaded onto a column (1500 g). The column was eluted with an ethyl acetate/cyclohexane gradient (0-100%), the clean fractions were combined and evaporated to give 1,1-dimethylethyl 7-(5-{3-cyano-4-[(1-methylethyl)oxy]phenyl}-1,2,4-oxadiazol-3-yl)-1,2,4,5-tetrahydro-3H-3-benzazepine-3-carboxylate as a white solid (81.5 g) and further product (3 g) was isolated from the solid stuck to the side of the large Buchi flask.

LCMS (Method formate): Retention time 1.51 min, no mass ion detected.

### Preparation 43

### 2-[(1-Methylethyl)oxy]-5-[3-(2,3,4,5-tetrahydro-1H-3-benzazepin-7-yl)-1,2,4-oxadiazol-5-yl]benzonitrile hydrochloride

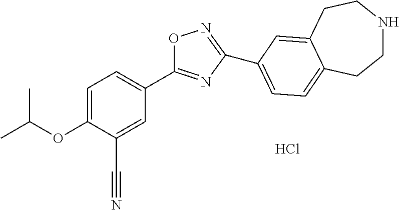

A solution of 1,1-dimethylethyl 7-(5-{3-cyano-4-[(1-methylethyl)oxy]phenyl}-1,2,4-oxadiazol-3-yl)-1,2,4,5-tetrahydro-3H-3-benzazepine-3-carboxylate (Preparation 42) (81 g, 170 mmol) in DCM (350 ml) was added dropwise to hydrogen chloride in 1,4-dioxane (4M, 427 ml, 1700 mmol) over 10 min. The resulting mixture was stirred for 3 h, then diethyl ether (1.5 l) was added and the resulting slurry stirred for 20 min. The solid product was isolated by filtration, washed with diethyl ether (2×300 ml) and dried under vacuum to give 2-[(1-methylethyl)oxy]-5-[3-(2,3,4,5-tetrahydro-1H-3-benzazepin-7-yl)-1,2,4-oxadiazol-5-yl]benzonitrile hydrochloride (73.9 g, 95%) as a colourless solid.

LCMS (Method formate): Retention time 0.92 min, MH⁺=375

### Preparation 44

Ethyl 4-[7-(5-{3-cyano-4-[(1-methylethyl)oxy]phenyl}-1,2,4-oxadiazol-3-yl)-1,2,4,5-tetrahydro-3H-3-benzazepin-3-yl]butanoate

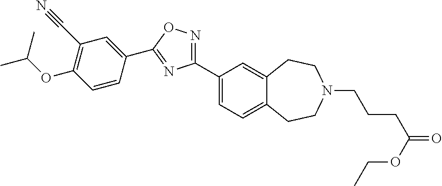

2-[(1-Methylethyl)oxy]-5-[3-(2,3,4,5-tetrahydro-1H-3-benzazepin-7-yl)-1,2,4-oxadiazol-5-yl]benzonitrile hydrochloride (Preparation 43) (51.0 g, 120 mmol) was added to stirred DMF (500 ml). To this suspension was added ethyl-4-bromobutyrate (27.5 g) followed by portionwise addition of potassium carbonate (42.9 g) and the reaction was heated to 90° C. for 4 h. The reaction was cooled to room temperature, diluted with water (1 l) and extracted with ethyl acetate (2×500 ml). The combined organic phases were washed with brine (6×500 ml) dried (Na₂SO₄) and evaporated to dryness. The residual gum was purified by chromatography eluting with ethyl acetate (70-100%). The clean fractions were combined and evaporated to give an off-white solid which was combined with alkylation product the reaction below. 2-[(1-Methylethyl)oxy]-5-[3-(2,3,4,5-tetrahydro-1H-3-benzazepin-7-yl)-1,2,4-oxadiazol-5-yl]benzonitrile hydrochloride (Preparation 43) (95.0 g, 231 mmol) was added to stirred DMF (1 l). To this suspension was added ethyl-4-bromobutyrate (51.1 g, 254 mmol) followed by portionwise addition of potassium carbonate (80 g, 578 mmol) and the resulting mixture was stirred at 90° C. for 4 h. The reaction was cooled to room temperature and diluted with water (2 l) and extracted with ethyl acetate (2×1 l). The combined organic layers were washed with brine (6×1 l), dried (Na₂SO₄) and evaporated to dryness. The residual gum was purified by chromatography eluting with ethyl acetate (70-100%). The clean fractions were combined with the solid from the reaction above and evaporated to dryness to give ethyl 4-[7-(5-{3-cyano-4-[(1-methylethyl)oxy]phenyl}-1,2,4-oxadiazol-3-yl)-1,2,4,5-tetrahydro-3H-3-benzazepin-3-yl]butanoate (125 g) as an off-white solid

LCMS (Method formate): Retention time 0.92 min, MH⁺=489

### Preparation 45

### 4-[7-(5-{3-Cyano-4-[(1-methylethyl)oxy]phenyl}-1,2,4-oxadiazol-3-yl)-1,2,4,5-tetrahydro-3H-3-benzazepin-3-yl]butanoic acid sodium salt

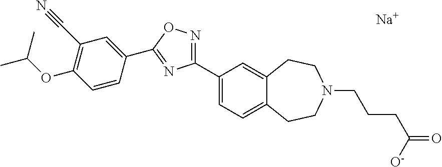

A suspension of ethyl 4-[7-(5-{3-cyano-4-[(1-methylethyl)oxy]phenyl}-1,2,4-oxadiazol-3-yl)-1,2,4,5-tetrahydro-3H-3-benzazepin-3-yl]butanoate (Preparation 44) (5.1 g, 10 mmol) in absolute ethanol (40 ml) was warmed until it went into solution. The solution was stirred and allowed to cool to 30° C. before addition of sodium hydroxide solution (2M, 10.4 ml). The mixture was stirred at room temperature for 2 h. The resulting suspension was cooled in ice and stirred for 30 min, then filtered and the solid washed with ethanol (20 ml). The solid was dried under vacuum at 40° C. to give a white solid. The filterate was evaporated to dryness and recrystallised from ethanol to give a white solid. Both solids were combined and recrystallised from hot ethanol to give 4-[7-(5-{3-cyano-4-[(1-methylethyl)oxy]phenyl}-1,2,4-oxadiazol-3-yl)-1,2,4,5-tetrahydro-3H-3-benzazepin-3-yl]butanoic acid sodium salt (2.73 g) as a pale cream powder.

LCMS (Method formate): Retention time 0.89 min, MH⁺=461

### Preparation 45: Alternative Procedure

### 4-[7-(5-{3-Cyano-4-[(1-methylethyl)oxy]phenyl}-1,2,4-oxadiazol-3-yl)-1,2,4,5-tetrahydro-3H-3-benzazepin-3-yl]butanoic acid sodium salt

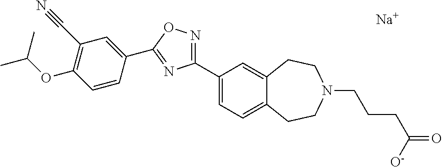

A suspension of ethyl 4-[7-(5-{3-cyano-4-[(1-methylethyl)oxy]phenyl}-1,2,4-oxadiazol-3-yl)-1,2,4,5-tetrahydro-3H-3-benzazepin-3-yl]butanoate (Preparation 44) (115 g, 240 mmol) in absolute ethanol (958 ml) was warmed until it went into solution. Upon cooling to 28° C. a fine colourless precipitate was formed. This mixture was treated with sodium hydroxide solution (2M, 235 ml, 471 mmol) added rapidly and with stirring. After stirring at room temperature for 2 h 50 min the reaction was cooled to 2° C. for 30 min and filtered. After overnight suction the solid was washed with cold ethanol (250 ml) and redried. The solid was suspended in ethanol (1 l), warmed to reflux, and filtered whilst hot. The filtrate was allowed to cool overnight and the white crystalline solid was isolated by filtration. The sample was dried at 40° C. for 5 h and further dried at 40° C. overnight to give 4-[7-(5-{3-cyano-4-[(1-methylethyl)oxy]phenyl}-1,2,4-oxadiazol-3-yl)-1,2,4,5-tetrahydro-3H-3-benzazepin-3-yl]butanoic acid sodium salt (52.9 g).

A portion (32.2 g) was taken from the bulk material and dried in vacuo at 50° C. over the weekend (32.2 g).

LCMS (Method formate): Retention time 0.98 min, MH⁺=461

### Preparation 45: Alternative Procedure

### 4-[7-(5-{3-Cyano-4-[(1-methylethyl)oxy]phenyl}-1,2,4-oxadiazol-3-yl)-1,2,4,5-tetrahydro-3H-3-benzazepin-3-yl]butanoic acid

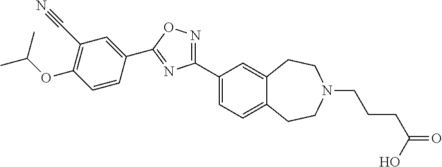

A suspension of ethyl 4-[7-(5-{3-cyano-4-[(1-methylethyl)oxy]phenyl}-1,2,4-oxadiazol-3-yl)-1,2,4,5-tetrahydro-3H-3-benzazepin-3-yl]butanoate (Preparation 44) (30.4 g, 62 mmol) in absolute ethanol (250 ml) was warmed until it went into solution and cooled. The resulting suspension was treated with sodium hydroxide solution (2M, 62 ml, 120 mmol) and the mixture stirred at room temperature for 3 h. The resulting suspension was filtered and the solid washed with ethanol (˜50 ml) and dried in a vacuum oven at 40° C. overnight. This material was dissolved in water (150 ml) and filtered. The filtrate was stirred in ice water and treated slowly with glacial acetic acid until the pH6 was obtained. The resulting solid lump was treated with more water (100 ml) and stirred for 1 h. This material was filtered, washed thoroughly with water (x2) and sucked dry. The resulting colourless solid was dried in a vacuum oven at 55° C. overnight. The solid was broken up and dried for a further 7 h. This was further crushed and dried overnight at 60° C. to give 4-[7-(5-{3-Cyano-4-[(1-methylethyl)oxy]phenyl}-1,2,4-oxadiazol-3-yl)-1,2,4,5-tetrahydro-3H-3-benzazepin-3-yl]butanoic acid as a pale cream powder (18.0 g, 63%).

LCMS (Method formate): Retention time 0.92 min, MH⁺=461

### Preparation 46

### 1,1-Dimethylethyl 7-(5-{3-cyano-4-[(1-methylethyl)oxy]phenyl}-1,3,4-thiadiazol-2-yl)-1,2,4,5-tetrahydro-3H-3-benzazepine-3-carboxylate

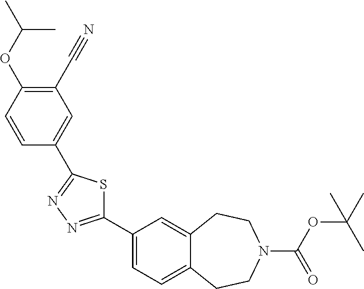

A mixture of 1,1-dimethylethyl 7-(4,4,5,5-tetramethyl-1,3,2-dioxaborolan-2-yl)-1,2,4,5-tetrahydro-3H-3-benzazepine-3-carboxylate (Preparation 39) (50 mg, 0.13 mmol, Wheaton Science Products), 5-(5-bromo-1,3,4-thiadiazol-2-yl)-2-[(1-methylethyl)oxy]benzonitrile (Preparation 2) (45 mg, 0.13 mmol), tetrakis(triphenylphosphine)palladium(0) (16 mg, 0.013 mmol) and tripotassium phosphate (71 mg, 0.34 mmol) in DMF (1.5 ml) and water (0.30 ml) was heated at 120° C. (microwave) for 20 min.

A mixture of 1,1-dimethylethyl 7-(4,4,5,5-tetramethyl-1,3,2-dioxaborolan-2-yl)-1,2,4,5-tetrahydro-3H-3-benzazepine-3-carboxylate (Preparation 39) (203 mg, 0.54 mmol, Wheaton Science Products), 5-(5-bromo-1,3,4-thiadiazol-2-yl)-2-[(1-methylethyl)oxy]benzonitrile (Preparation 2) (176 mg, 0.54 mmol), tetrakis(triphenylphosphine)palladium(0) (63 mg, 0.054 mmol) and tripotassium phosphate (289 mg, 1.4 mmol) in DMF (4.5 ml) and water (0.9 ml) was heated at 120° C. (microwave) for 20 min. This reaction mixture was combined with the experiment above and partitioned between ethyl acetate (50 ml) and water (50 ml). The aqueous phase was extracted with ethyl acetate (2×50 ml), the combined organic phases were washed with brine (2×50 ml) and dried (MgSO₄) and concentrated. The residue was dissolved in DCM and applied to a silica cartridge, the cartridge was eluted with ethyl acetate/cyclohexane gradient (0-50%) to give, after evaporation of the solvent from the product fractions in vacuo, 1,1-dimethylethyl 7-(5-{3-cyano-4-[(1-methylethyl)oxy]phenyl}-1,3,4-thiadiazol-2-yl)-1,2,4,5-tetrahydro-3H-3-benzazepine-3-carboxylate (246 mg,) as a colourless solid.

LCMS (Method formate): Retention time 1.46 min, MH⁺=491

### Preparation 47

### 2-[(1-Methylethyl)oxy]-5-[5-(2,3,4,5-tetrahydro-1H-3-benzazepin-7-yl)-1,3,4-thiadiazol-2-yl]benzonitrile trifluoroacetic salt

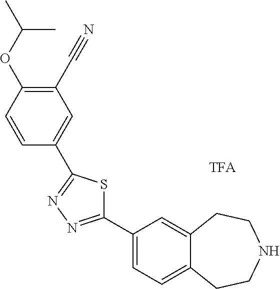

Trifluoroacetic acid (0.19 ml, 2.5 mmol) was added to a solution of 1,1-dimethylethyl 7-(5-{3-cyano-4-[(1-methylethyl)oxy]phenyl}-1,3,4-thiadiazol-2-yl)-1,2,4,5-tetrahydro-3H-3-benzazepine-3-carboxylate (Preparation 46) (246 mg, 0.50 mmol) in DCM (5 ml) and the mixture was stirred at room temperature for 18 h then concentrated. Trituration of the residue with diethyl ether (5 ml) gave 2-[(1-methylethyl)oxy]-5-[5-(2,3,4,5-tetrahydro-1H-3-benzazepin-7-yl)-1,3,4-thiadiazol-2-yl]benzonitrile trifluoroacetic salt (296 mg, 111%) as a colourless solid which was used in the next step without further purification.

LCMS (Method formate): Retention time 0.81 min, MH⁺=391

### Preparation 48

### 1,1-Dimethylethyl 6-{[(trifluoromethyl)sulfonyl]oxy}-1,2,4,5-tetrahydro-3H-3-benzazepine-3-carboxylate

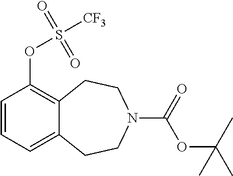

Trifluoromethanesulfonic acid anhydride (0.18 ml, 1.1 mmol) was added dropwise to a solution of 1,1-dimethylethyl 6-hydroxy-1,2,4,5-tetrahydro-3H-3-benzazepine-3-carboxylate (189 mg, 0.72 mmol, WO2006/002928) in pyridine (3 ml) at −5° C. under nitrogen over 5 min. The mixture was left at −5° C. for 15 min.

Trifluoromethanesulfonic acid anhydride (2.21 ml, 13 mmol) was added dropwise to a solution of 1,1-dimethylethyl 6-hydroxy-1,2,4,5-tetrahydro-3H-3-benzazepine-3-carboxylate (2.30 g, 8.7 mmol, WO2006/002928) in pyridine (15 ml) at −5° C. under nitrogen over 5 min. The mixture was stirred at −5° C. for 30 min, combined with the reaction above and concentrated in vacuo. The residue was partitioned between ethyl acetate (3×50 ml) and a hydrochloric acid (50 ml) and the layers separated. The aqueous phase was extracted twice with AcOEt (1M, 50 ml). The combined organic extracts were washed with a saturated sodium hydrogen carbonate (50 ml), then brine (50 ml), dried (MgSO₄) and concentrated to give 1,1-dimethylethyl 6-{[(trifluoromethyl)sulfonyl]oxy}-1,2,4,5-tetrahydro-3H-3-benzazepine-3-carboxylate (3.44 g, 90%) as a buff-coloured solid.

LCMS (Method formate): Retention time 1.41 min, MH⁺=396

### Preparation 49

### 1,1-Dimethylethyl 6-cyano-1,2,4,5-tetrahydro-3H-3-benzazepine-3-carboxylate

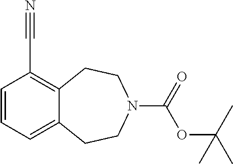

Tetrakis(triphenylphosphine)palladium(0) (58 mg, 0.051 mmol) was added to a mixture of 1,1-dimethylethyl 6-{[(trifluoromethyl)sulfonyl]oxy}-1,2,4,5-tetrahydro-3H-3-benzazepine-3-carboxylate (Preparation 48) (0.2 g, 0.51 mmol) and zinc cyanide (89 mg, 0.76 mmol) DMF (5 ml) under nitrogen and the mixture heated at 100° C. for 2 h. Further zinc cyanide (89 mg, 0.76 mmol) and tetrakis(triphenylphosphine)palladium(0) (58 mg, 0.051 mmol) were added and the mixture heated at 120° C. for 20 min. Further zinc cyanide (89 mg, 0.76 mmol) and tetrakis(triphenylphosphine)palladium(0) (58 mg, 0.051 mmol) were added and heating continued at 130° C. for 30 min. The reaction mixture was combined with the reaction below.

To a mixture of 1,1-dimethylethyl 6-{[(trifluoromethyl)sulfonyl]oxy}-1,2,4,5-tetrahydro-3H-3-benzazepine-3-carboxylate (Preparation 48) (0.1 g, 0.25 mmol) which had been previously purified by chromatography and zinc cyanide (0.045 g, 0.38 mmol) in DMF (5 ml) under nitrogen was added tetrakis(triphenylphosphine)palladium(0) (29 mg, 0.025 mmol) and the mixture heated at 130° C. (microwave) for 30 min. A further portion of zinc cyanide (45 mg, 0.38 mmol) and tetrakis(triphenylphosphine)palladium(0) (29 mg, 0.025 mmol) were added and heating continued for 30 min. The reaction mixture was combined with the reaction below.

To a mixture of 1,1-dimethylethyl 6-{[(trifluoromethyl)sulfonyl]oxy}-1,2,4,5-tetrahydro-3H-3-benzazepine-3-carboxylate (Preparation 48) (1.40 g, 3.5 mmol) which had been previously purified by chromatography and zinc cyanide (0.624 g, 5.3 mmol) in DMF (25 ml) under nitrogen was added tetrakis(triphenylphosphine)palladium(0) (0.409 g, 0.35 mmol). The mixture was divided into 2 aliquots and heated at 140° C. for 30 min (microwave). The cooled mixtures were concentrated in vacuo and the residue partitioned between water (50 ml) and ethyl acetate (3×25 ml). The combined organic extracts were washed with brine (50 ml) and dried (MgSO₄). The solvent was evaporated to give an orange oil. The crude product was dissolved in DCM, applied to a silica cartridge and the cartridge eluted with an ethyl acetate/cyclohexane gradient (0-50%). The appropriate fractions were combined and evaporated in vacuo to give 1,1-dimethylethyl 6-cyano-1,2,4,5-tetrahydro-3H-3-benzazepine-3-carboxylate (898 mg) as a pale yellow gum.

LCMS (Method formate): Retention time 1.17 min, MH⁺=273 (weak)

### Preparation 50

### 1,1-Dimethylethyl 6-[(hydroxyamino)(imino)methyl]-1,2,4,5-tetrahydro-3H-3-benzazepine-3-carboxylate

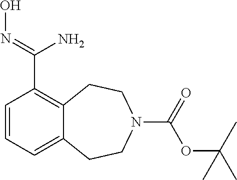

Hydroxylamine hydrochloride (0.92 g, 13 mmol) was added to a mixture of 1,1-dimethylethyl 6-cyano-1,2,4,5-tetrahydro-3H-3-benzazepine-3-carboxylate (Preparation 49) (0.90 g, 3.3 mmol, WO2009080725) and sodium hydrogen carbonate (1.66 g, 20 mmol) in ethanol (20 ml) and the mixture stirred at 70° C. over a weekend, cooled and concentrated in vacuo. The residue was partitioned between water (20 ml) and ethyl acetate (20 ml). The aqueous phase was extracted with ethyl acetate (2×20 ml). The combined organic extracts were washed with brine (50 ml), dried (MgSO₄) and concentrated to give 1,1-dimethylethyl 6-[(hydroxyamino)(imino)methyl]-1,2,4,5-tetrahydro-3H-3-benzazepine-3-carboxylate (920 mg, 59%) as a colourless solid which was used in the next step without further purification.

LCMS (Method formate): Retention time 0.71 min, MH⁺=306

### Preparation 51

### 1,1-Dimethylethyl 6-(5-{3-cyano-4-[(1-methylethyl)oxy]phenyl}-1,2,4-oxadiazol-3-yl)-1,2,4,5-tetrahydro-3H-3-benzazepine-3-carboxylate

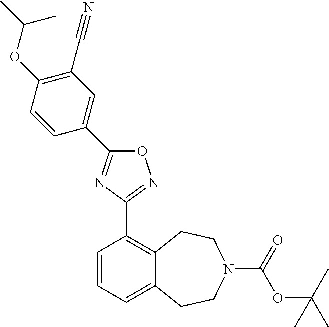

3-Cyano-4-[(1-methylethyl)oxy]benzoyl chloride (Preparation 169) (88 mg, 0.39 mmol, WO2009080730/WO2008128951) was added to a solution of 1,1-dimethylethyl 6-[(hydroxyamino)(imino)methyl]-1,2,4,5-tetrahydro-3H-3-benzazepine-3-carboxylate (Preparation 50) (100 mg, 0.33 mmol) and triethylamine (0.068 ml, 0.49 mmol) in DMF) (3 ml) at room temperature under nitrogen. The mixture was stirred for 30 min then heated at 120° C. for 30 min. The reaction was combined with the experiment below.

3-Cyano-4-[(1-methylethyl)oxy]benzoyl chloride (Preparation 169) (713 mg, 3.2 mmol, WO2009080730/WO2008128951) was added to a solution of 1,1-dimethylethyl 6-[(hydroxyamino)(imino)methyl]-1,2,4,5-tetrahydro-3H-3-benzazepine-3-carboxylate (Preparation 50) (810 mg, 2.7 mmol) and triethylamine (0.56 ml, 4.0 mmol) in DMF (20 ml) at room temperature under nitrogen. The mixture was stirred for 30 min at this temperature and then at 120° C. for 2 h. The cooled reaction was combined with the reaction above and partitioned between water (30 ml) and ethyl acetate (30 ml). The aqueous phase was extracted with ethyl acetate (2×30 ml). The combined organic phases were washed with brine/water 1:1 (3×30 ml) and dried (MgSO₄) and concentrated. The residue was dissolved in DCM and loaded onto a silica cartridge, which was eluted with an ethyl acetate/cyclohexane gradient (0 to 50% ethyl acetate). Evaporation of the solvents from the product fractions in vacuo gave 1,1-dimethylethyl 6-(5-{3-cyano-4-[(1-methylethyl)oxy]phenyl}-1,2,4-oxadiazol-3-yl)-1,2,4,5-tetrahydro-3H-3-benzazepine-3-carboxylate (810 mg, 62%) as a yellow solid.

LCMS (Method formate): Retention time 1.48 min, MH⁺=475

### Preparation 52

### 2-[(1-Methylethyl)oxy]-5-[5-(2,3,4,5-tetrahydro-1H-3-benzazepin-6-yl)-1,2,4-oxadiazol-5-yl]benzonitrile trifluoroacetic acid salt

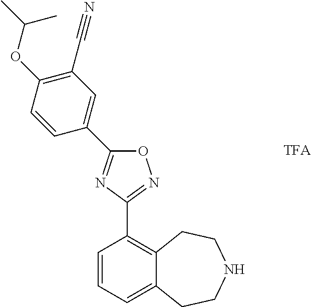

Trifluoroacetic acid (0.65 ml, 8.4 mmol) was added to a solution of 1,1-dimethylethyl 6-(5-{3-cyano-4-[(1-methylethyl)oxy]phenyl}-1,2,4-oxadiazol-3-yl)-1,2,4,5-tetrahydro-3H-3-benzazepine-3-carboxylate (Preparation 51) (800 mg, 1.7 mmol) in DCM (15 ml) at 0° C. and the mixture allowed to warm to room temperature and stirred overnight. The solvent was then concentrated to give 2-[(1-methylethyl)oxy]-5-[3-(2,3,4,5-tetrahydro-1H-3-benzazepin-6-yl)-1,2,4-oxadiazol-5-yl]benzonitrile trifluoroacetic acid salt (970 mg, 109%) as a colourless solid which was used in the next step without further purification.

LCMS (Method formate): Retention time 0.91 min, MH⁺=375

### Preparation 53

### 1,1-Dimethylethyl 5-bromo-3,4-dihydro-2(1H)-isoquinolinecarboxylate

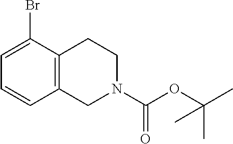

5-Bromo-1,2,3,4-tetrahydroisoquinoline hydrobromide (2.24 g, 9.0 mmol, Zannan Pharma), di-tert-butyl dicarbonate (3.1 ml, 14 mmol) and triethylamine (2.8 ml, 20 mmol) were dissolved in THF (40 ml) and the mixture was stirred at room temperature for 1 h then concentrated in vacuo. Water (15 ml) was added to the residue and the mixture extracted with ethyl acetate (2×15 ml). The combined organic phases were dried (Na₂SO₄) and concentrated in vacuo to give 1,1-dimethylethyl 5-bromo-3,4-dihydro-2(1H)-isoquinolinecarboxylate (2.79 g, 99%) as an off-white solid which was used in the next step without further purification.

LCMS (Method formate, 5 min): Retention time 3.60 min, MH⁺=312/314

### Preparation 54

### 1,1-Dimethylethyl 5-(4,4,5,5-tetramethyl-1,3,2-dioxaborolan-2-yl)-3,4-dihydro-2(1H)-isoquinolinecarboxylate

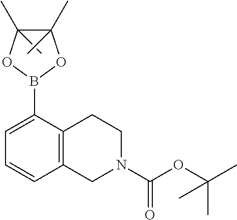

1,1-Dimethylethyl 5-bromo-3,4-dihydro-2(1H)-isoquinolinecarboxylate (Preparation 53) (2.7 g, 8.7 mmol), potassium acetate (2.55 g, 26 mmol), [1,1′-bis(diphenylphosphino)ferrocene]dichloropalladium(II) (0.63 g, 0.87 mmol) and 4,4,4′,4′,5,5,5′,5′-octamethyl-2,2′-bi-1,3,2-dioxaborolane (4.39 g, 17 mmol) were dissolved in 1,4-dioxane (40 ml), stirred at 80° C. under nitrogen for 2 h and allowed to cool. Water (30 ml) was added and the mixture was extracted with ethyl acetate (3×20 ml). The combined organic phases were concentrated in vacuo. Purification of the residue by flash chromatography (ethyl acetate in cyclohexane 15%) gave 1,1-dimethylethyl 5-(4,4,5,5-tetramethyl-1,3,2-dioxaborolan-2-yl)-3,4-dihydro-2(1H)-isoquinolinecarboxylate as a clear gel (3.25 g, 105%).

LCMS (Method formate): Retention time 1.51 min, MH⁺=360

### Preparation 55

### 1,1-Dimethylethyl 5-(5-{3-cyano-4-[(1-methylethyl)oxy]phenyl}-1,3,4-thiadiazol-2-yl)-3,4-dihydro-2(1H)-isoquinolinecarboxylate

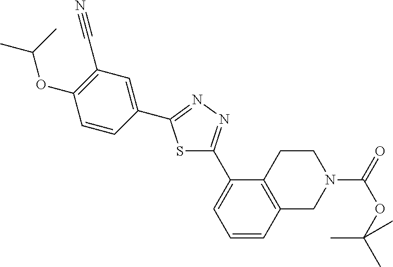

1,1-Dimethylethyl 5-(4,4,5,5-tetramethyl-1,3,2-dioxaborolan-2-yl)-3,4-dihydro-2(1H)-isoquinolinecarboxylate (Preparation 54) (1.0 g, 2.8 mmol), 5-(5-bromo-1,3,4-thiadiazol-2-yl)-2-[(1-methylethyl)oxy]benzonitrile (Preparation 2) (1.35 g, 4.2 mmol), dichlorobis(triphenylphosphine)-palladium (II) (0.20 g, 0.28 mmol) and sodium carbonate (1.48 g, 14 mmol) were dissolved in 1,2-dimethoxyethane (7.5 ml) and water (2.5 ml). The resulting mixture was stirred at 120° C. for 40 min (microwave). Dichlorobis(triphenylphosphine)-palladium (II) (0.20 g, 0.28 mmol) was added and the resulting mixture was stirred at 120° C. for 40 min (microwave). The mixture was diluted with water (20 ml) and extracted with ethyl acetate (3×15 ml). The combined organic phases were dried (Na₂SO₄) and concentrated in vacuo. Purification of the residue by chromatography (ethyl acetate/cyclohexane, 0 to 30% gradient) gave 1,1-dimethylethyl 5-(5-{3-cyano-4-[(1-methylethyl)oxy]phenyl}-1,3,4-thiadiazol-2-yl)-3,4-dihydro-2(1H)-isoquinolinecarboxylate (290 mg, 22%) as a brown solid.

LCMS (Method formate): Retention time 1.43 min, MH⁺=477

### Preparation 56

### 2-[(1-Methylethyl)oxy]-5-[5-(1,2,3,4-tetrahydro-5-isoquinolinyl)-1,3,4-thiadiazol-2-yl]benzonitrile hydrochloride

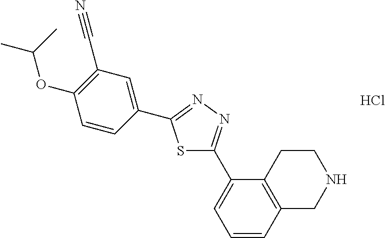

1,1-Dimethylethyl 5-(5-{3-cyano-4-[(1-methylethyl)oxy]phenyl}-1,3,4-thiadiazol-2-yl)-3,4-dihydro-2(1H)-isoquinolinecarboxylate (Preparation 55) (320 mg, 0.67 mmol) was dissolved in a mixture of 1,4-dioxane (4 ml) and hydrogen chloride in 1,4-dioxane (4M, 4 ml, 16 mmol. The resulting mixture was stirred at room temperature for 3 h. Ether was then added and the mixture stirred for 10 min. The precipitate was isolated by filtration and rinsed with diethyl ether to give 2-[(1-methylethyl)oxy]-5-[5-(1,2,3,4-tetrahydro-5-isoquinolinyl)-1,3,4-thiadiazol-2-yl]benzonitrile hydrochloride (260 mg, 92%) as a cream coloured solid.

LCMS (Method HpH): Retention time 1.12 min, MH⁺=377

### Preparation 57

### 1,1-Dimethylethyl {3-[6-(5-{3-cyano-4-[(1-methylethyl)oxy]phenyl}-1,2,4-oxadiazol-3-yl)-5-methyl-3,4-dihydro-2(1H)-isoquinolinyl]-3-oxopropyl}carbamate

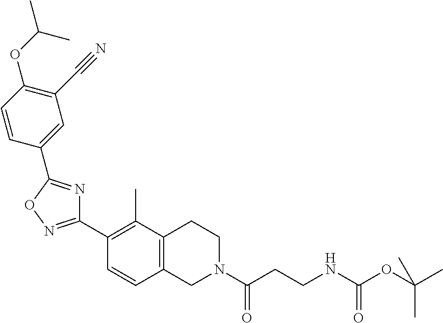

A mixture of N-{[(1,1-dimethylethyl)oxy]carbonyl}-b-alanine (63 mg, 0.33 mmol, Avocado), N-ethylmorpholine (84 μl, 0.67 mmol), HOBT hydrate (61 mg, 0.4 mmol), EDC (77 mg, 0.4 mmol) and 2-[(1-methylethyl)oxy]-5-[3-(5-methyl-1,2,3,4-tetrahydro-6-isoquinolinyl)-1,2,4-oxadiazol-5-yl]benzonitrile trifluoroacetic acid salt (Preparation 25) (150 mg) in DMF (5 ml) was stirred at room temperature overnight. The reaction was diluted with saturated sodium hydrogen carbonate solution (10 ml), extracted with ethyl acetate (2×10 ml). The combined organic extracts were washed with water then brine, dried and concentrated. Purification of the residue by chromatography (ethyl acetate/iso-hexane, 10%) gave 1,1-dimethylethyl {3-[6-(5-{3-cyano-4-[(1-methylethyl)oxy]phenyl}-1,2,4-oxadiazol-3-yl)-5-methyl-3,4-dihydro-2(1H)-isoquinolinyl]-3-oxopropyl}carbamate (150 mg) as a colourless solid.

LCMS (Method formate): Retention time 1.31 min, MH⁺=546

### Preparation 58

### 1,1-Dimethylethyl {3-[6-(5-{3-cyano-4-[(1-methylethyl)oxy]phenyl}-1,2,4-oxadiazol-3-yl)-5-methyl-3,4-dihydro-2(1H)-isoquinolinyl]-3-oxopropyl}methylcarbamate

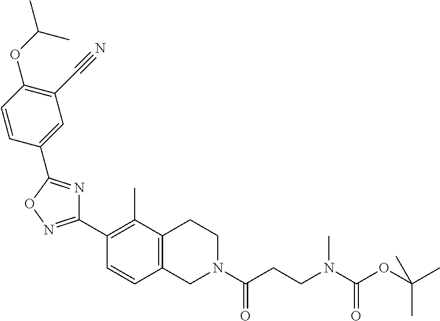

A mixture of N-{[(1,1-dimethylethyl)oxy]carbonyl}-N-methyl-b-alanine (52 mg, 0.26 mmol, Aldrich), N-ethylmorpholine (97 μl, 0.78 mmol), HOBT hydrate (47 mg, 0.31 mmol), EDC (59 mg, 0.31 mmol) and 2-[(1-methylethyl)oxy]-5-[3-(5-methyl-1,2,3,4-tetrahydro-6-isoquinolinyl)-1,2,4-oxadiazol-5-yl]benzonitrile trifluoroacetic acid salt (Preparation 25) (150 mg, 0.31 mmol) in DMF (5 ml) was stirred at room temperature overnight. The reaction was diluted with saturated sodium hydrogen carbonate solution (10 ml) and extracted with ethyl acetate (2×10 ml). The combined organic extracts were washed with water then brine, dried and concentrated. Purification of the residue by chromatography (ethyl acetate/iso-hexane, 10%) gave 1,1-dimethylethyl {3-[6-(5-{3-cyano-4-[(1-methylethyl)oxy]phenyl}-1,2,4-oxadiazol-3-yl)-5-methyl-3,4-dihydro-2(1H)-isoquinolinyl]-3-oxopropyl}methylcarbamate (130 mg, 88%) as a colourless oil which solidified.

LCMS (Method formate): Retention time 1.35 min, MH⁺=560

### Preparation 59

### 1,1-Dimethylethyl {4-[6-(5-{3-cyano-4-[(1-methylethyl)oxy]phenyl}-1,2,4-oxadiazol-3-yl)-5-methyl-3,4-dihydro-2(1H)-isoquinolinyl]-4-oxobutyl}carbamate

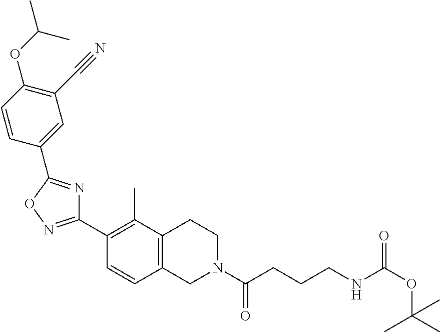

A mixture of 4-({[(1,1-dimethylethyl)oxy]carbonyl}amino)butanoic acid (52 mg, 0.26 mmol, Aldrich), N-ethylmorpholine (97 μl, 0.78 mmol), HOBT (47 mg, 0.31 mmol), EDC (59 mg, 0.31 mmol) and 2-[(1-methylethyl)oxy]-5-[3-(5-methyl-1,2,3,4-tetrahydro-6-isoquinolinyl)-1,2,4-oxadiazol-5-yl]benzonitrile trifluoroacetic acid salt (Preparation 25) (150 mg, 0.31 mmol) in DMF (5 ml) was stirred at room temperature overnight. The reaction was diluted with saturated sodium hydrogen carbonate solution (10 ml), extracted with ethyl acetate (2×10 ml). The combined organic extracts were washed with water then brine, dried and concentrated. Purification of the residue by chromatography (ethyl acetate/iso-hexane, 10%) gave 1,1-dimethylethyl {4-[6-(5-{3-cyano-4-[(1-methylethyl)oxy]phenyl}-1,2,4-oxadiazol-3-yl)-5-methyl-3,4-dihydro-2(1H)-isoquinolinyl]-4-oxobutyl}carbamate (110 mg, 79%) as a colourless oil which solidified.

LCMS (Method formate): Retention time 1.31 min, MH⁺=560

### Preparation 60

### 1,1-Dimethylethyl {2-[6-(5-{3-cyano-4-[(1-methylethyl)oxy]phenyl}-1,2,4-oxadiazol-3-yl)-5-methyl-3,4-dihydro-2(1H)-isoquinolinyl]-2-oxoethyl}carbamate

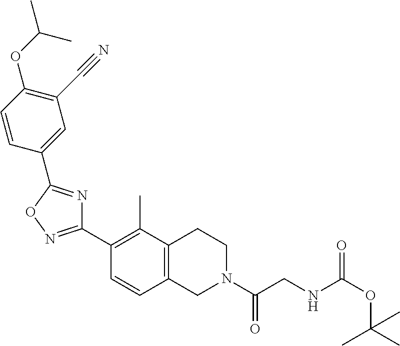

To a solution of N-{[(1,1-dimethylethyl)oxy]carbonyl}glycine (96 mg, 0.55 mmol) in DMF (4 ml) at room temperature were added EDC (115 mg, 0.60 mmol), HOBT (100 mg, 0.65 mmol) then N-ethylmorpholine (0.19 ml, 1.5 mmol) and after 2 min, 2-[(1-methylethyl)oxy]-5-[3-(5-methyl-1,2,3,4-tetrahydro-6-isoquinolinyl)-1,2,4-oxadiazol-5-yl]benzonitrile hydrochloric acid salt (Preparation 25) (205 mg, 0.5 mmol). The resulting mixture was stirred at room temperature overnight then concentrated in vacuo. The residue was dissolved in ethyl acetate, washed with saturated sodium hydrogen carbonate solution. The aqueous phase was extracted with ethyl acetate and the combined organic phases washed with brine, dried (MgSO₄) and concentrated in vacuo. Purification of the residue by chromatography (ethyl acetate/cyclohexane) gave 1,1-dimethylethyl {2-[6-(5-{3-cyano-4-[(1-methylethyl)oxy]phenyl}-1,2,4-oxadiazol-3-yl)-5-methyl-3,4-dihydro-2(1H)-isoquinolinyl]-2-oxoethyl}carbamate (203 mg, 76%) as a white foam.

LCMS (Method HpH): Retention time 1.35 min, MH⁺=532

### Preparation 61

### 2-[(1-methylethyl)oxy]-5-[5-(1,2,3,4-tetrahydro-6-isoquinolinyl)-1,3,4-thiadiazol-2-yl]benzonitrile

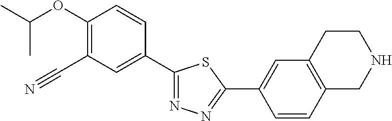

Trifluoroacetic acid (0.5 ml, 6.5 mmol) was added to a solution of 1,1-dimethylethyl 6-(5-{3-cyano-4-[(1-methylethyl)oxy]phenyl}-1,3,4-thiadiazol-2-yl)-3,4-dihydro-2(1H)-isoquinolinecarboxylate (Preparation 62) (380 mg, 0.80 mmol) in DCM (4 ml) at 0° C. under nitrogen. The mixture was allowed to warm slowly to room temperature After 1 h, trifluoroacetic acid (0.5 ml) was added and stirring continued for a further 3 h. The mixture was evaporated and the residual oil dissolved in in methanol and applied to an SCX SPE (20 g). The SPE was eluted with methanol, then ammonia in methanol (2M). The ammonia fractions were combined and evaporated to give 2-[(1-methylethyl)oxy]-5-[5-(1,2,3,4-tetrahydro-6-isoquinolinyl)-1,3,4-thiadiazol-2-yl]benzonitrile as a pale yellow oil (326 mg).

LCMS (Method formate): Retention time 0.85 min, MH⁺=377

### Preparation 62

### 1,1-dimethylethyl 6-(5-{3-cyano-4-[(1-methylethyl)oxy]phenyl}-1,3,4-thiadiazol-2-yl)-3,4-dihydro-2(1H)-isoquinolinecarboxylate

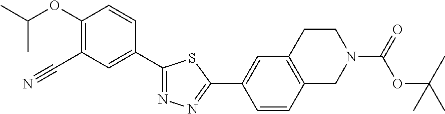

5-(5-Bromo-1,3,4-thiadiazol-2-yl)-2-[(1-methylethyl)oxy]benzonitrile (Preparation 2) (500 mg, 1.5 mmol) and 1,1-dimethylethyl 6-(4,4,5,5-tetramethyl-1,3,2-dioxaborolan-2-yl)-3,4-dihydro-2(1H)-isoquinolinecarboxylate (Preparation 63) (580 mg, 1.6 mmol) were suspended in 1,4-dioxane (6 ml) and saturated sodium hydrogen carbonate solution (1.5 ml, 1.6 mmol) under nitrogen. The reaction mixture was degassed, 1,1′-bis(diphenylphosphino)ferrocenedichloro palladium(II) dichloromethane complex (120 mg, 0.16 mmol) added and the mixture degassed again. The reaction was heated at 90° C. under nitrogen for 5 h, cooled to room temperature and quenched with saturated sodium hydrogen carbonate solution. The mixture was diluted with ethyl acetate (10 ml) and filtered to remove solids. The aqueous phase was extracted with ethyl acetate (2×25 ml), the combined organics washed with saturated brine, dried (MgSO₄) and evaporated to give a dark brown oil which was stored under vacuum overnight. The residue was purified by chromatography on a silica cartridge, eluting with an ethyl acetate/hexane gradient (5-50%). The desired fractions were combined and evaporated to give 1,1-dimethylethyl 6-(5-{3-cyano-4-[(1-methylethyl)oxy]phenyl}-1,3,4-thiadiazol-2-yl)-3,4-dihydro-2(1H)-isoquinolinecarboxylate as a yellow solid (390 mg).

LCMS (Method formate): Retention time 1.42 min, MH⁺=477

### Preparation 63

### 1,1-dimethylethyl 6-(4,4,5,5-tetramethyl-1,3,2-dioxaborolan-2-yl)-3,4-dihydro-2(1H)-isoquinolinecarboxylate

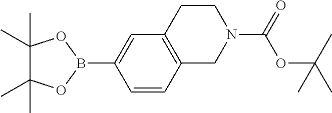

1,1-Dimethylethyl 6-bromo-3,4-dihydro-2(1H)-isoquinolinecarboxylate (Preparation 64) (1.22 g, 3.9 mmol) and bis(pinacolato)diboron (1.09 g, 4.3 mmol) were dissolved in 1,4-dioxane (15 ml), the mixture degassed under nitrogen at room temperature. 1,1′-Bis(diphenylphosphino)ferrocenedichloro palladium(II) dichloromethane complex (0.17 g, 0.23 mmol) and 1,1′-bis(diphenylphosphino)ferrocene (0.13 g, 0.23 mmol) were added the mixture stirred at room temperature for 5 min. Potassium acetate (1.15 g, 12 mmol) was added and the mixture heated at 90° C. under nitrogen for 4 h. The reaction was allowed to cool to room temperature overnight, quenched with saturated sodium hydrogen carbonate solution (20 ml) and filtered. This filtrate was extracted with ethyl acetate (2×20 ml), the combined organics washed with saturated brine, dried (MgSO₄) and evaporated to give a dark brown oil. This oil was absorbed onto a 40+ samplet and dried in the vacuum oven, The samplet was washed with DCM/methanol This solution was evaporated and the brown residue was purified by chromatography on a silica cartridge eluting with an ethyl acetate/hexane gradient (5-50%). Product fractions were combined and evaporated to give 1,1-dimethylethyl 6-(4,4,5,5-tetramethyl-1,3,2-dioxaborolan-2-yl)-3,4-dihydro-2(1H)-isoquinolinecarboxylate as a pale yellow oil (580 mg). Stored in the freezer over the weekend to give a white solid

LCMS (Method formate): Retention time 1.46 min, MH⁺ not observed

### Preparation 64

### 1,1-dimethylethyl 6-bromo-3,4-dihydro-2(1H)-isoquinolinecarboxylate

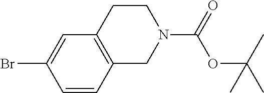

Triethylamine (2.8 ml, 20 mmol) was added to a suspension of 6-bromo-1,2,3,4-tetrahydroisoquinoline hydrochloride (1 g, 4.0 mmol, ASW MedChem Product List) and di-tert-butyl dicarbonate (1.87 ml, 8.1 mmol) in methanol (10 ml) at room temperature under nitrogen. The mixture was stirred overnight and then for a further 6 h. The solvent was evaporated to give a white solid, which was partitioned between DCM and saturated sodium hydrogen carbonate solution, the organic dried (hydrophobic frit), and concentrated. The residue was dried under vacuum overnight, dissolved in methanol and applied to an SCX SPE (20 g). The cartridge was eluted with methanol and the fractions combined and evaporated to give 1,1-dimethylethyl 6-bromo-3,4-dihydro-2(1H)-isoquinolinecarboxylate as a pale yellow gum (1.22 g).

LCMS (Method formate): Retention time 1.38 min, MH⁺=312/314.

### Preparation 65

### 1,1-dimethylethyl {2-[7-(5-{3-cyano-4-[(1-methylethyl)oxy]phenyl}-1,2,4-oxadiazol-3-yl)-1,2,4,5-tetrahydro-3H-3-benzazepin-3-yl]-2-oxoethyl}carbamate

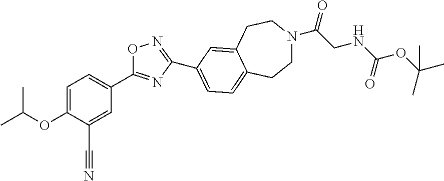

N-{[(1,1-Dimethylethyl)oxy]carbonyl}glycine (50 mg, 0.29 mmol) was dissolved in DMF (5 ml), then N-ethylmorpholine (0.11 ml, 0.86 mmol), HOBT (53 mg, 0.34 mmol) and EDC (66 mg, 0.34 mmol) were added. The mixture was stirred at room temperature for 10 min, then 2-[(1-methylethyl)oxy]-5-[3-(2,3,4,5-tetrahydro-1H-3-benzazepin-7-yl)-1,2,4-oxadiazol-5-yl]benzonitrile hydrochloride (Preparation 43) (140 mg, 0.34 mmol) was added. The mixture was stirred at room temperature for 1 h and the reaction mixture was partitioned between DCM (5 ml) and saturated sodium hydrogen carbonate solution (10 ml). The aqueous was extracted with DCM (5 ml), the organics combined, dried (hydrophobic frit) and reduced to dryness under a stream of nitrogen to give 1,1-dimethylethyl {2-[7-(5-{3-cyano-4-[(1-methylethyl)oxy]phenyl}-1,2,4-oxadiazol-3-yl)-1,2,4,5-tetrahydro-3H-3-benzazepin-3-yl]-2-oxoethyl}carbamate as a yellow gum (200 mg). This was used without further purification is subsequent steps.

LCMS (Method formate): Retention time 1.30 min, MH⁺=532

### Preparation 66

### 2-[(1-methylethyl)amino]-5-[3-(2,3,4,5-tetrahydro-1H-3-benzazepin-7-yl)-1,2,4-oxadiazol-5-yl]benzonitrile

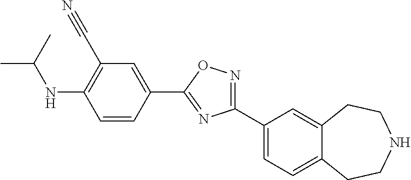

To 1,1-dimethylethyl 7-(5-{3-cyano-4-[(1-methylethyl)amino]phenyl}-1,2,4-oxadiazol-3-yl)-1,2,4,5-tetrahydro-3H-3-benzazepine-3-carboxylate (Preparation 67) (230 mg, 0.48 mmol) in DCM (10 ml) was added trifluoroacetic acid (2.5 ml, 32 mmol) and the reaction mixture stirred at room temperature for 1 h. The reaction mixture was evaporated in vacuo and the residue applied to an aminopropyl SPE (5 g). The SPE was eluted using methanol in DCM (10%). The appropriate fractions were combined and dried under a stream of nitrogen to give 2-[(1-methylethyl)amino]-5-[3-(2,3,4,5-tetrahydro-1H-3-benzazepin-7-yl)-1,2,4-oxadiazol-5-yl]benzonitrile (180 mg). LCMS (Method formate): Retention time 0.87 min, MH⁺=374

### Preparation 67

### 1,1-dimethylethyl 7-(5-{3-cyano-4-[(1-methylethyl)amino]phenyl}-1,2,4-oxadiazol-3-yl)-1,2,4,5-tetrahydro-3H-3-benzazepine-3-carboxylate

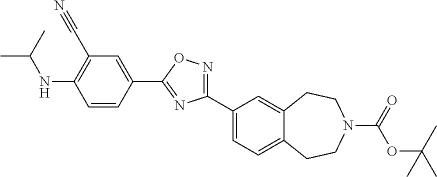

To isopropylamine (0.24 ml, 2.8 mmol) in DMF (1 ml) at room temperature was added sodium hydride (60% in mineral oil, 150 mg, 3.7 mmol) and the reaction stirred for 10 min. 1,1-Dimethylethyl 7-[5-(3-cyano-4-fluorophenyl)-1,2,4-oxadiazol-3-yl]-1,2,4,5-tetrahydro-3H-3-benzazepine-3-carboxylate (Preparation 68) (400 mg, 0.92 mmol) in DMF (1 ml) was added and the reaction was stirred overnight. The reaction mixture was partitioned between ethyl acetate (50 ml) and saturated aqueous sodium hydrogen carbonate solution (50 ml) and the aqueous extracted with ethyl acetate (50 ml). The organics were combined, dried (hydrophobic frit) and reduced to dryness in vacuo. The sample was dissolved in DCM, loaded onto a silica cartridge (100 g) and eluted using an ethyl acetate/cyclohexane gradient (0-30%) followed by continued elution at ethyl acetate/cyclohexane (30%). The appropriate fractions were combined and dried under a stream of nitrogen to give the 1,1-dimethylethyl 7-(5-{3-cyano-4-[(1-methylethyl)amino]phenyl}-1,2,4-oxadiazol-3-yl)-1,2,4,5-tetrahydro-3H-3-benzazepine-3-carboxylate (225 mg).

LCMS (Method formate): Retention time 1.52 min, MH⁺ not seen.

### Preparation 68

### 1,1-dimethylethyl 7-[5-(3-cyano-4-fluorophenyl)-1,2,4-oxadiazol-3-yl]-1,2,4,5-tetrahydro-3H-3-benzazepine-3-carboxylate

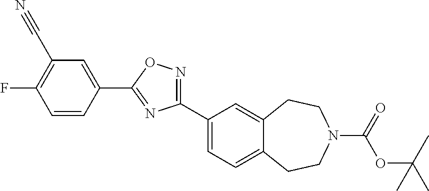

To a suspension of 1,1-dimethylethyl 7-[(hydroxyamino)(imino)methyl]-1,2,4,5-tetrahydro-3H-3-benzazepine-3-carboxylate (Preparation 41) (4.61 g, 15 mmol) in toluene (30 ml) and pyridine (30 ml) was added slowly 3-cyano-4-fluorobenzoyl chloride (Preparation 69) (2.9 g, 16 mmol) in toluene (10 ml+2 ml rinse). After 30 min, the resulting mixture was stirred under a gentle reflux (bath at 120° C.) for 1.5 h. The mixture was cooled to room temperature, and most of the solvent evaporated.

The residue was partially dissolved in a mixture of ethyl acetate and hydrochloric acid (2N). The mixture filtered, the phases separated and the aqueous extracted with ethyl acetate. The combined organic phases were washed with sodium hydrogen carbonate, brine, dried (MgSO₄) and concentrated in vacuo. Trituration of the residue with diethyl ether/DCM gave 1,1-dimethylethyl 7-[5-(3-cyano-4-fluorophenyl)-1,2,4-oxadiazol-3-yl]-1,2,4,5-tetrahydro-3H-3-benzazepine-3-carboxylate (2.65 g, 6.10 mmol, 40.4%) as a orange solid. The mother liquor was concentrated to give an orange foam (2.36 g) and purified by flash chromatography on a silica cartridge, eluting with an ethyl acetate/cyclohexane gradient to give 1,1-dimethylethyl 7-[5-(3-cyano-4-fluorophenyl)-1,2,4-oxadiazol-3-yl]-1,2,4,5-tetrahydro-3H-3-benzazepine-3-carboxylate (1.9 g, 29%) as a white solid.

LCMS of orange solid (Method formate): Retention time 1.44 min, MH⁺=not seen

LCMS of white solid (Method HpH): Retention time 1.47 min, MH⁺=not seen

### Preparation 69

### 3-cyano-4-fluorobenzoyl chloride

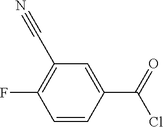

To a suspension of 3-cyano-4-fluorobenzoic acid (4.8 g, 29 mmol) in DCM (70 ml) at room temperature was added oxalyl chloride (3.31 ml, 38 mmol) then DMF (0.11 ml, 1.453 mmol). After ca 15 min, the solvent was evaporated and the residue co-evaporated with toluene to give 3-cyano-4-fluorobenzoyl chloride (5.4 g 101%) as an orange solid which was used in the next step without further purification.

### Preparation 70

### 2-(propylamino)-5-[3-(2,3,4,5-tetrahydro-1H-3-benzazepin-7-yl)-1,2,4-oxadiazol-5-yl]benzonitrile

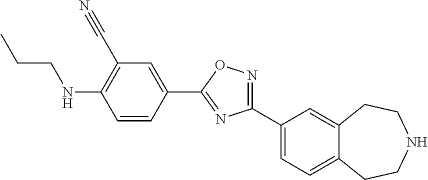

To 1,1-dimethylethyl 7-{5-[3-cyano-4-(propylamino)phenyl]-1,2,4-oxadiazol-3-yl}-1,2,4,5-tetrahydro-3H-3-benzazepine-3-carboxylate (Preparation 71) (230 mg, 0.48 mmol) in DCM (10 ml) was added trifluoroacetic acid (2.5 ml, 32 mmol) and the reaction mixture stirred at room temperature for 1 h. The reaction mixture was evaporated in vacuo and the residue was applied to an aminopropyl SPE (5 g) and eluted using methanol in DCM (10%). The appropriate fractions were combined and dried under a stream of nitrogen to give 2-(propylamino)-5-[3-(2,3,4,5-tetrahydro-1H-3-benzazepin-7-yl)-1,2,4-oxadiazol-5-yl]benzonitrile (119 mg).

LCMS (Method formate): Retention time 0.88 min, MH⁺=374

### Preparation 71

### 1,1-dimethylethyl 7-{5-[3-cyano-4-(propylamino)phenyl]-1,2,4-oxadiazol-3-yl}-1,2,4,5-tetrahydro-3H-3-benzazepine-3-carboxylate

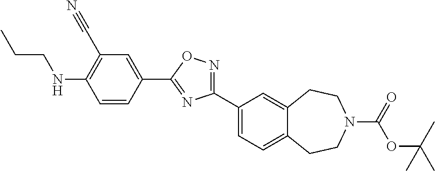

To n-propylamine (0.23 ml, 2.8 mmol) in DMF (1 ml) at room temperature was added sodium hydride (60% in mineral oil, 150 mg, 3.7 mmol) and the reaction stirred for 10 min. 1,1-dimethylethyl 7-[5-(3-cyano-4-fluorophenyl)-1,2,4-oxadiazol-3-yl]-1,2,4,5-tetrahydro-3H-3-benzazepine-3-carboxylate (Preparation 68) (400 mg, 0.92 mmol) in DMF (1 ml) was added and the reaction stirred overnight. The reaction mixture was partitioned between ethyl acetate (2×50 ml) and saturated aqueous sodium hydrogen carbonate solution (30 ml). The organics were combined, dried (hydrophobic frit) and evaporated in vacuo. The sample was dissolved in DCM and loaded on to a silica cartridge (100 g) and the cartridge eluted with an ethyl acetate/cyclohexane (0-30%), followed by continued elution with ethyl acetate/cyclohexane (30%). The appropriate fractions were combined and concentrated under a stream of nitrogen to give 1,1-dimethylethyl 7-{5-[3-cyano-4-(propylamino)phenyl}-1,2,4-oxadiazol-3-yl]-1,2,4,5-tetrahydro-3H-3-benzazepine-3-carboxylate as an off-white solid (230 mg).

LCMS (Method formate): Retention time 1.52 min, MH⁺=not seen.

### Preparation 72

### 2-(propyloxy)-5-[3-(2,3,4,5-tetrahydro-1H-3-benzazepin-7-yl)-1,2,4-oxadiazol-5-yl]benzonitrile

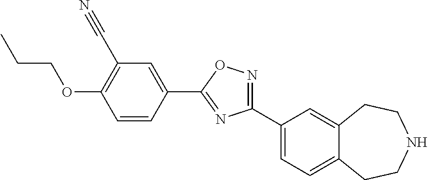

To 1,1-dimethylethyl 7-{5-[3-cyano-4-(propyloxy)phenyl]-1,2,4-oxadiazol-3-yl}-1,2,4,5-tetrahydro-3H-3-benzazepine-3-carboxylate (Preparation 73) (180 mg, 0.38 mmol) in DCM (10 ml) was added trifluoroacetic acid (2.5 ml, 32 mmol) and the reaction mixture stirred at room temperature for 30 min. The reaction mixture was evaporated in vacuo and the residue dissolved in DCM and applied to an aminopropyl SPE (5 g) and the SPE eluted using methanol in DCM (10%). The appropriate fractions were combined and reduced to dryness under a stream of nitrogen to give 2-(propyloxy)-5-[3-(2,3,4,5-tetrahydro-1H-3-benzazepin-7-yl)-1,2,4-oxadiazol-5-yl]benzonitrile (100 mg).

LCMS (Method formate): Retention time 0.86 min, MH⁺=375

### Preparation 73

### 1,1-dimethylethyl 7-{5-[3-cyano-4-(propyloxy)phenyl]-1,2,4-oxadiazol-3-yl}-1,2,4,5-tetrahydro-3H-3-benzazepine-3-carboxylate

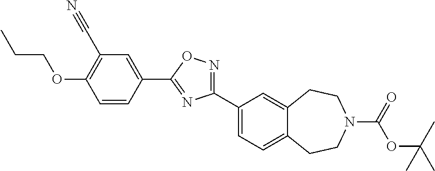

To n-propanol (0.21 ml, 2.8 mmol) in DMF (1 ml) at room temperature was added sodium hydride (60% in mineral oil, 150 mg, 3.7 mmol) and the reaction stirred for 10 min. 1,1-dimethylethyl 7-[5-(3-cyano-4-fluorophenyl)-1,2,4-oxadiazol-3-yl]-1,2,4,5-tetrahydro-3H-3-benzazepine-3-carboxylate (Preparation 68) (400 mg, 0.92 mmol) in DMF (1 ml) was added and the reaction was stirred overnight. The reaction mixture was partitioned between ethyl acetate (2×50 ml) and saturated aqueous sodium hydrogen carbonate solution (50 ml). The organics were combined, dried (hydrophobic frit) and evaporated in vacuo. The sample was dissolved in DCM, loaded onto a silica cartridge (100 g) and the cartridge eluted using an ethyl acetate/cyclohexane gradient (0-30%), followed by continued elution with ethyl acetate/cyclohexane (30%). The appropriate fractions were combined and reduced to dryness under a stream of nitrogen to give 1,1-dimethylethyl 7-{5-[3-cyano-4-(propyloxy)phenyl}-1,2,4-oxadiazol-3-yl]-1,2,4,5-tetrahydro-3H-3-benzazepine-3-carboxylate as an off-white solid (180 mg).

LCMS (Method formate): Retention time 1.51 min, MH⁺=not seen

### Preparation 74

### 1,1-dimethylethyl {2-[7-(5-{3-cyano-4-[(1-methylethyl)amino]phenyl}-1,2,4-oxadiazol-3-yl)-1,2,4,5-tetrahydro-3H-3-benzazepin-3-yl]-2-oxoethyl}carbamate

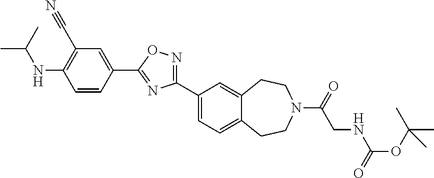

To N-{[(1,1-dimethylethyl)oxy]carbonyl}glycine (51 mg, 0.29 mmol), HOBT (56 mg, 0.37 mmol) and EDC (70 mg, 0.37 mmol) in DMF (3 ml) was added N-ethylmorpholine (0.093 ml, 0.73 mmol) and the reaction mixture stirred at room temperature for 10 min. 2-[(1-Methylethyl)amino]-5-[3-(2,3,4,5-tetrahydro-1H-3-benzazepin-7-yl)-1,2,4-oxadiazol-5-yl]benzonitrile (Preparation 66) (91 mg, 0.24 mmol) was added and the reaction mixture stirred at room temperature for 1 h. The reaction mixture was reduced to dryness under a stream of nitrogen and the residue partitioned between DCM (2×10 ml) and saturated aqueous sodium hydrogen carbonate solution (10 ml). The organics were combined, dried (hydrophobic frit) and the solvent evaporated under a stream of nitrogen to give 1,1-dimethylethyl {2-[7-(5-{3-cyano-4-[(1-methylethyl)amino]phenyl}-1,2,4-oxadiazol-3-yl)-1,2,4,5-tetrahydro-3H-3-benzazepin-3-yl]-2-oxoethyl}carbamate which was used without further purification in the next reaction (Example 52).

LCMS (Method formate): Retention time 1.34 min, MH⁺=531

### Preparation 75

### 1,1-dimethylethyl [2-(7-{5-[3-cyano-4-(propylamino)phenyl}-1,2,4-oxadiazol-3-yl]-1,2,4,5-tetrahydro-3H-3-benzazepin-3-yl)-2-oxoethyl]carbamate

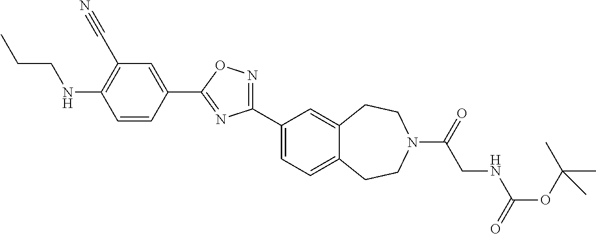

To N-{[(1,1-dimethylethyl)oxy]carbonyl}glycine (33 mg, 0.190 mmol), HOBT (36 mg, 0.24 mmol) and EDC (45 mg, 0.24 mmol) in DMF (3 ml) was added N-ethylmorpholine (0.060 ml, 0.47 mmol) and the reaction mixture stirred at room temperature for 10 min. 2-(Propylamino)-5-[3-(2,3,4,5-tetrahydro-1H-3-benzazepin-7-yl)-1,2,4-oxadiazol-5-yl]benzonitrile (Preparation 70) (59 mg, 0.16 mmol) was added and the reaction mixture stirred at room temperature for 1 h. The reaction mixture was reduced to dryness under a stream of nitrogen and the residue partitioned between DCM (2×10 ml) and saturated aqueous sodium hydrogen carbonate solution (10 ml). The organics were combined, dried (hydrophobic frit) and evaporated under a stream of nitrogen to give 1,1-dimethylethyl [2-(7-{5-[3-cyano-4-(propylamino)phenyl}-1,2,4-oxadiazol-3-yl]-1,2,4,5-tetrahydro-3H-3-benzazepin-3-yl)-2-oxoethyl]carbamate, which was used without further preparation in the next reaction (Example 53).

LCMS (Method formate): Retention time 1.34 min, MH⁺=531

### Preparation 76

### 1,1-dimethylethyl (2-{7-[5-(3-cyano-4-{[2-fluoro-1-(fluoromethyl)ethyl]oxy}phenyl)-1,2,4-oxadiazol-3-yl]-1,2,4,5-tetrahydro-3H-3-benzazepin-3-yl}-2-oxoethyl)carbamate

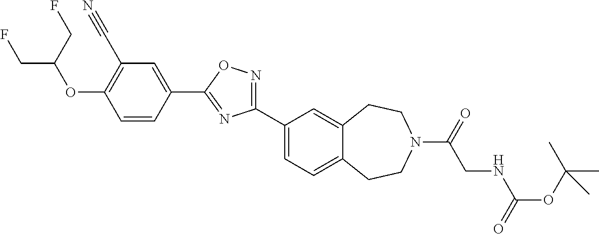

To N-{[(1,1-dimethylethyl)oxy]carbonyl}glycine (72 mg, 0.41 mmol), HOBT (78 mg, 0.51 mmol) and EDC (98 mg, 0.51 mmol) in DMF (3 ml) was added N-ethylmorpholine (0.13 ml, 1.0 mmol) and the reaction mixture stirred at room temperaure for 10 min. 2-{[2-Fluoro-1-(fluoromethyl)ethyl]oxy}-5-[3-(2,3,4,5-tetrahydro-1H-3-benzazepin-7-yl)-1,2,4-oxadiazol-5-yl]benzonitrile (Preparation 77) (140 mg, 0.34 mmol) was added and the reaction mixture stirred at room temperature for 1 h. The reaction mixture was reduced to dryness under a stream of nitrogen and the residue partitioned between DCM (2×10 ml) and saturated aqueous sodium hydrogen carbonate solution (10 ml). The organics were combined, dried (hydrophobic frit) and evaporated under a stream of nitrogen to give 1,1-dimethylethyl (2-{7-[5-(3-cyano-4-{[2-fluoro-1-(fluoromethyl)ethyl)oxy}phenyl)-1,2,4-oxadiazol-3-yl]-1,2,4,5-tetrahydro-3H-3-benzazepin-3-yl}-2-oxoethyl)carbamate, which was used without further purification in the next reaction (Example 54).

LCMS (Method formate): Retention time 1.19 min, MH⁺=568

### Preparation 77

### 2-{[2-Fluoro-1-(fluoromethyl)ethyl]oxy}-5-[3-(2,3,4,5-tetrahydro-1H-3-benzazepin-7-yl)-1,2,4-oxadiazol-5-yl]benzonitrile

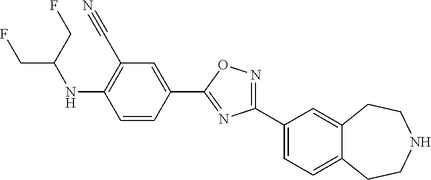

To 1,1-dimethylethyl 7-[5-(3-cyano-4-{[2-fluoro-1-(fluoromethyl)ethyl)oxy}phenyl)-1,2,4-oxadiazol-3-yl]-1,2,4,5-tetrahydro-3H-3-benzazepine-3-carboxylate (Preparation 78) (380 mg, 0.74 mmol) in DCM (10 ml) was added trifluoroacetic acid (2.5 ml, 32 mmol) and the reaction mixture stirred at room temperature for 30 min. The reaction mixture was concentrated in vacuo and the residue dissolved in DCM and applied to an aminopropyl SPE (5 g) and the SPE eluted using methanol in DCM (10%). The appropriate fractions were combined and dried under a stream of nitrogen to give 2-{[2-fluoro-1-(fluoromethyl)ethyl)oxy}-5-[3-(2,3,4,5-tetrahydro-1H-3-benzazepin-7-yl)-1,2,4-oxadiazol-5-yl]benzonitrile (300 mg).

LCMS (Method formate): Retention time 0.80 min, MH⁺=411

### Preparation 78

### 1,1-dimethylethyl 7-[5-(3-cyano-4-{[2-fluoro-1-(fluoromethyl)ethyl]oxy}phenyl)-1,2,4-oxadiazol-3-yl]-1,2,4,5-tetrahydro-3H-3-benzazepine-3-carboxylate

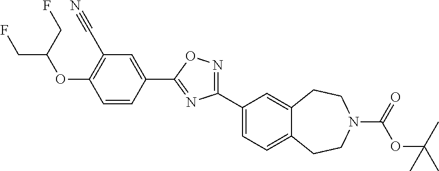

To 1,3-difluoro-2-propanol (0.24 ml, 3.1 mmol) in DMF (1 ml) at room temperature was added sodium hydride (60% in mineral oil, 170 mg, 4.1 mmol) and the reaction stirred for 10 min. 1,1-Dimethylethyl 7-[5-(3-cyano-4-fluorophenyl)-1,2,4-oxadiazol-3-yl]-1,2,4,5-tetrahydro-3H-3-benzazepine-3-carboxylate (Preparation 68) (450 mg, 1.0 mmol) in DMF (1 ml) was added and the reaction was stirred overnight. The reaction mixture was partitioned between ethyl acetate (2×50 ml) and saturated aqueous sodium hydrogen carbonate solution (50 ml). The organics were combined, dried (hydrophobic frit) and reduced to dryness in vacuo. The sample was dissolved in DCM, applied to a silica cartridge (100 g) and eluted with an ethyl acetate/cyclohexane gradient (0-30%), followed by continued elution with ethyl acetate/cyclohexane (30%). The appropriate fractions were combined and reduced to dryness under a stream of nitrogen to give 1,1-dimethylethyl 7-[5-(3-cyano-4-{[2-fluoro-1-(fluoromethyl)ethyl)oxy]phenyl)-1,2,4-oxadiazol-3-yl]-1,2,4,5-tetrahydro-3H-3-benzazepine-3-carboxylate (380 mg).

LCMS (Method formate): Retention time 1.38 min, MH⁺=not seen.

### Preparation 79

### 2-(3-fluoro-1-pyrrolidinyl)-5-[3-(2,3,4,5-tetrahydro-1H-3-benzazepin-7-yl)-1,2,4-oxadiazol-5-yl]benzonitrile

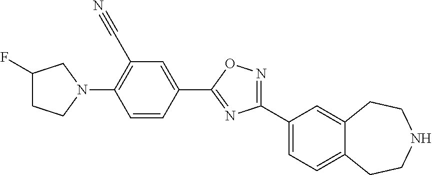

To 1,1-dimethylethyl 7-{5-[3-cyano-4-(3-fluoro-1-pyrrolidinyl)phenyl]-1,2,4-oxadiazol-3-yl]-1,2,4,5-tetrahydro-3H-3-benzazepine-3-carboxylate (Preparation 80) (150 mg, 0.30 mmol) in DCM (10 ml) was added trifluoroacetic acid (0.023 ml, 0.30 mmol) and the reaction mixture stirred at room temperature for 30 min. The reaction was reduced to dryness in vacuo, the residue dissolved in DCM, applied to an aminopropyl SPE (5 g) and the SPE eluted using methanol in DCM (10%). The appropriate fractions were combined and reduced to dryness under a stream of nitrogen to give 2-(3-fluoro-1-pyrrolidinyl)-5-[3-(2,3,4,5-tetrahydro-1H-3-benzazepin-7-yl)-1,2,4-oxadiazol-5-yl]benzonitrile (76 mg).

LCMS (Method formate): Retention time 0.81 min, MH⁺=404

### Preparation 80

### 1,1-dimethylethyl 7-{5-[3-cyano-4-(3-fluoro-1-pyrrolidinyl)phenyl]-1,2,4-oxadiazol-3-yl}-1,2,4,5-tetrahydro-3H-3-benzazepine-3-carboxylate

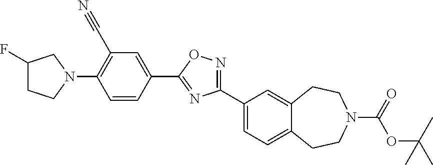

To 3-fluoropyrrolidine hydrochloride salt (350 mg, 2.8 mmol) in DMF (1 ml) at room temperature was added sodium hydride (60% in mineral oil, 180 mg, 4.6 mmol) and the reaction stirred for 10 min. 1,1-Dimethylethyl 7-[5-(3-cyano-4-fluorophenyl)-1,2,4-oxadiazol-3-yl]-1,2,4,5-tetrahydro-3H-3-benzazepine-3-carboxylate (Preparation 68) (400 mg, 0.92 mmol) in DMF (1 ml) was added and the reaction stirred overnight. The reaction mixture was partitioned between ethyl acetate (2×50 ml) and saturated aqueous sodium hydrogen carbonate solution (50 ml). The organics were combined, dried (hydrophobic frit) and evaporated in vacuo. The residue was dissolved in DCM and loaded onto a silica cartridge (100 g) which was eluted with an ethyl acetate/cyclohexane gradient (0-30%) followed by continued elution with ethyl acetate/cyclohexane (30%). The appropriate fractions were combined and reduced to dryness under a stream of nitrogen to give 1,1-dimethylethyl 7-{5-[3-cyano-4-(3-fluoro-1-pyrrolidinyl)phenyl}-1,2,4-oxadiazol-3-yl]-1,2,4,5-tetrahydro-3H-3-benzazepine-3-carboxylate as an off-white solid (150 mg).

LCMS (Method formate): Retention time 1.44 min, MH⁺ not seen.

### Preparation 81

### 1,1-dimethylethyl [2-(7-{5-[3-cyano-4-(3-fluoro-1-pyrrolidinyl)phenyl}-1,2,4-oxadiazol-3-yl}-1,2,4,5-tetrahydro-3H-3-benzazepin-3-yl)-2-oxoethyl]carbamate

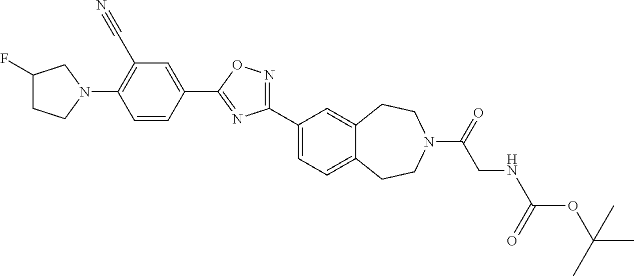

To N-{[(1,1-dimethylethyl)oxy]carbonyl}glycine (20 mg, 0.11 mmol), HOBT (22 mg, 0.14 mmol) and EDC (27 mg, 0.14 mmol) in DMF (3 ml) was added N-ethylmorpholine (0.036 ml, 0.28 mmol) and the reaction mixture stirred at room temperature for 10 min. 2-(3-Fluoro-1-pyrrolidinyl)-5-[3-(2,3,4,5-tetrahydro-1H-3-benzazepin-7-yl)-1,2,4-oxadiazol-5-yl]benzonitrile (Preparation 79) (38 mg) was added and the reaction mixture stirred at room temperature for 1 h. The reaction mixture was reduced to dryness under a stream of nitrogen and the residue partitioned between DCM (2×10 ml) and saturated aqueous sodium hydrogen carbonate solution (10 ml). The organics were combined, dried (hydrophobic frit) and reduced to dryness under a stream of nitrogen to give 1,1-dimethylethyl [2-(7-{5-[3-cyano-4-(3-fluoro-1-pyrrolidinyl)phenyl]-1,2,4-oxadiazol-3-yl}-1,2,4,5-tetrahydro-3H-3-benzazepin-3-yl)-2-oxoethyl]carbamate which was used without further purification in the next reaction (Example 57).

LCMS (Method formate): Retention time 1.26 min, MH⁺=561

### Preparation 82

### 1,1-dimethylethyl [2-(7-{5-[3-cyano-4-(ethyloxy)phenyl]-1,2,4-oxadiazol-3-yl)-1,2,4,5-tetrahydro-3H-3-benzazepin-3-yl)-2-oxoethyl]carbamate

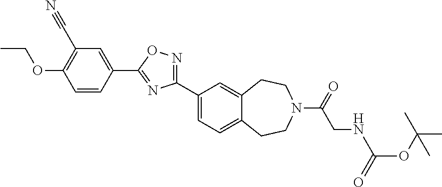

To N-{[(1,1-dimethylethyl)oxy]carbonyllglycine (66 mg, 0.38 mmol), HOBT (72 mg, 0.47 mmol) and EDC (90 mg, 0.47 mmol) in DMF (3 ml) was added N-ethylmorpholine (0.12 ml, 0.94 mmol) and the reaction mixture was stirred at room temperature for 10 min. 2-(Ethyloxy)-5-[3-(2,3,4,5-tetrahydro-1H-3-benzazepin-7-yl)-1,2,4-oxadiazol-5-yl]benzonitrile (Preparation 83) (110 mg, 0.31 mmol) was added and the reaction mixture stirred at room temperature for 1 h. The reaction was reduced to dryness under a stream of nitrogen and the residue partitioned between DCM (2×10 ml) and saturated aqueous sodium hydrogen carbonate solution (10 ml). The organics were combined, dried (hydrophobic frit) and evaporated under a stream of nitrogen to give 1,1-dimethylethyl [2-(7-{5-[3-cyano-4-(ethyloxy)phenyl]-1,2,4-oxadiazol-3-yl}-1,2,4,5-tetrahydro-3H-3-benzazepin-3-yl)-2-oxoethyl]carbamate which was used without further purification in the next reaction.

LCMS (Method formate): Retention time 1.34 min, MH⁺=531

### Preparation 83

### 2-(Ethyloxy)-5-[3-(2,3,4,5-tetrahydro-1H-3-benzazepin-7-yl)-1,2,4-oxadiazol-5-yl]benzonitrile

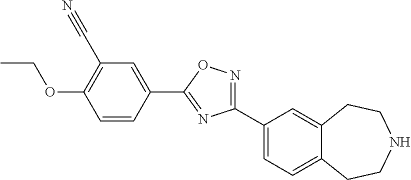

To 1,1-dimethylethyl 7-{5-[3-cyano-4-(ethyloxy)phenyl]-1,2,4-oxadiazol-3-yl}-1,2,4,5-tetrahydro-3H-3-benzazepine-3-carboxylate (Preparation 84) (350 mg, 0.76 mmol) in DCM (10 ml) was added trifluoroacetic acid (2.5 ml, 32 mmol) and the reaction mixture stirred at room temperature for 30 min. The reaction was concentrated in vacuo and the residue dissolved in DCM and applied to an aminopropyl SPE (5 g). The SPE was eluted using methanol in DCM (10%) and the appropriate fractions were combined and reduced to dryness under a stream of nitrogen to give 2-(ethyloxy)-5-[3-(2,3,4,5-tetrahydro-1H-3-benzazepin-7-yl)-1,2,4-oxadiazol-5-yl]benzonitrile (200 mg).

LCMS (Method formate): Retention time 0.83 min, MH⁺=361

### Preparation 84

### 1,1-dimethylethyl 7-{5-[3-cyano-4-(ethyloxy)phenyl]-1,2,4-oxadiazol-3-yl}-1,2,4,5-tetrahydro-3H-3-benzazepine-3-carboxylate

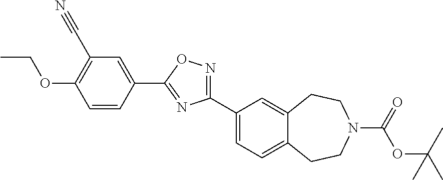

To ethanol (0.18 ml, 3.11 mmol) in DMF (1 ml) at room temperature was added sodium hydride (60% in mineral oil, 170 mg, 4.1 mmol) and the reaction stirred for 10 min. 1,1-Dimethylethyl 7-[5-(3-cyano-4-fluorophenyl)-1,2,4-oxadiazol-3-yl]-1,2,4,5-tetrahydro-3H-3-benzazepine-3-carboxylate (Preparation 68) (450 mg, 1.0 mmol) in DMF (1 ml) was added and the reaction was stirred overnight. The reaction mixture was partitioned between ethyl acetate (2×50 ml) and saturated aqueous sodium hydrogen carbonate solution (50 ml). The organics were combined, dried (hydrophobic frit) and evaporated in vacuo. The residue was dissolved in DCM, loaded onto a silica cartridge (100 g) and the cartridge eluted with an ethyl acetate/cyclohexane gradient (0-30% ethyl acetate) followed by continued elution with ethyl acetate/cyclohexane (30%). The appropriate fractions were combined and reduced to dryness under a stream of nitrogen to give 1,1-dimethylethyl 7-{5-[3-cyano-4-(ethyloxy)phenyl]-1,2,4-oxadiazol-3-yl}-1,2,4,5-tetrahydro-3H-3-benzazepine-3-carboxylate (350 mg).

LCMS (Method formate): Retention time 1.45 min, MH⁺ not seen. [M-tBu+MeCN]H+=446

### Preparation 85

### 1,1-dimethylethyl [2-(7-{5-[3-cyano-4-(propyloxy)phenyl]-1,2,4-oxadiazol-3-yl}-1,2,4,5-tetrahydro-3H-3-benzazepin-3-yl)-2-oxoethyl]carbamate

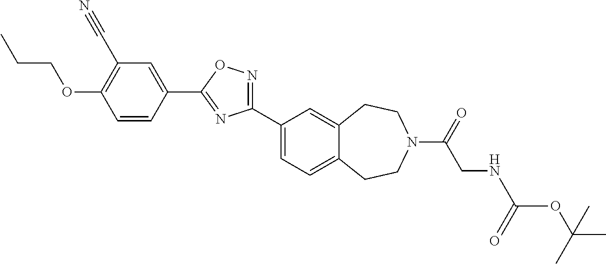

To N-{[(1,1-dimethylethyl)oxy}carbonyllglycine (29 mg, 0.16 mmol), HOBT (31 mg, 0.20 mmol) and EDC (39 mg, 0.20 mmol) in DMF (3 ml) was added N-ethylmorpholine (0.052 ml, 0.41 mmol) and the reaction mixture stirred at room temperature for 10 min. 2-(Propyloxy)-5-[3-(2,3,4,5-tetrahydro-1H-3-benzazepin-7-yl)-1,2,4-oxadiazol-5-yl]benzonitrile (Preparation 72) (51 mg, 0.14 mmol) was added and the reaction mixture stirred at room temperature for 1 h. The reaction mixture was reduced to dryness under a stream of nitrogen and the residue partitioned between DCM (2×10 ml) and saturated aqueous sodium hydrogen carbonate solution (10 ml). The organics were combined, dried (hydrophobic frit) and evaporated under a stream of nitrogen to give 1,1-dimethylethyl [2-(7-{5-[3-cyano-4-(propyloxy)phenyl]-1,2,4-oxadiazol-3-yl}-1,2,4,5-tetrahydro-3H-3-benzazepin-3-yl)-2-oxoethyl]carbamate which was used without further purification in the next reaction (Example 62).

LCMS (Method formate): Retention time 1.34 min, WI⁺=532

### Preparation 86

### 5-{3-[2-(2,2-dimethyl-1,3-dioxan-5-yl)-5-methyl-1,2,3,4-tetrahydro-6-isoquinolinyl]-1,2,4-oxadiazol-5-yl}-2-[(1-methylethyl)oxy]benzonitrile

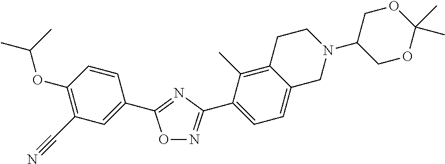

To a stirred suspension of 2-[(1-methylethyl)oxy]-5-[3-(5-methyl-1,2,3,4-tetrahydro-6-isoquinolinyl)-1,2,4-oxadiazol-5-yl]benzonitrile hydrochloride (Preparation 25) (186 mg, 0.45 mmol) in DCM (4 ml) under nitrogen was added 2,2-dimethyl-1,3-dioxan-5-one (0.11 ml, 0.91 mmol) followed by sodium triacetoxyborohydride (296 mg, 1.4 mmol) portionwise. The mixture was stirred under nitrogen at room temperature for 2 h. Further 2,2-dimethyl-1,3-dioxan-5-one (0.054 ml, 0.22 mmol) then sodium triacetoxyborohydride (147 mg, 0.34 mmol) were added to the mixture which was stirred at room temperature for a further 18 h. Saturated aqueous sodium carbonate solution (5 ml) was added and the mixture stirred vigorously for 15 min. The phases were separated and the aqueous phase was extracted with DCM (3×4 ml). The combined organic phases were dried (hydrophobic frit) and the solvent was evaporated under a stream of nitrogen to give 5-{3-[2-(2,2-dimethyl-1,3-dioxan-5-yl)-5-methyl-1,2,3,4-tetrahydro-6-isoquinolinyl]-1,2,4-oxadiazol-5-yl}-2-[(1-methylethyl)oxy]benzonitrile as a cream solid (276 mg). This material was used in the subsequent reaction without further purification (Example 64).

LCMS (Method formate): Retention time 1.06 min, MH⁺=489

### Preparation 86: Alternative Procedure

### 5-{3-[2-(2,2-dimethyl-1,3-dioxen-5-yl)-5-methyl-1,2,3,4-tetrahydro-6-isoquinolinyl]-1,2,4-oxadiazol-5-yl}-2-[(1-methylethyl)oxy]benzonitrile

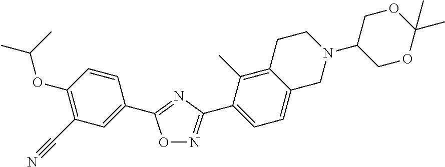

Sodium triacetoxyborohydride (74.3 g, 350 mmol) was added in small portions over 30 min to a mixture of 2,2-dimethyl-1,3-dioxan-5-one (30.4 g, 230 mmol) and 2-[(1-methylethyl)oxy]-5-[3-(5-methyl-1,2,3,4-tetrahydro-6-isoquinolinyl)-1,2,4-oxadiazol-5-yl]benzonitrile hydrochloride (Preparation 25) (32 g, 78 mmol) in DCM (400 ml) at room temperature. The mixture was stirred under nitrogen for 4 h, then, added cautiously to a solution of sodium hydrogen carbonate (140 g) in water (1.2 l). The phases were separated and the organic layer washed with water, brine, dried and evaporated to give a beige solid. The crude product was suspended in ethanol (300 ml) and heated to reflux with vigorous stirring for 30 min, then cooled in an ice bath and the product collected by filtration to give 5-{3-[2-(2,2-dimethyl-1,3-dioxan-5-yl)-5-methyl-1,2,3,4-tetrahydro-6-isoquinolinyl]-1,2,4-oxadiazol-5-yl}-2-[(1-methylethyl)oxy]benzonitrile (33 g, 87%).

NMR (CDCl₃): δ H 8.42 (1H, d), 8.33 (1H, dd), 7.74 (1H, d), 7.12 (1H, d), 7.03 (1H, d), 4.79 (1H, m), 4.12-4.08 (2H, m), 3.97-3.92 (2H, m), 3.85 (2H, s), 2.96 (2H, t), 2.86-2.80 (3H, m), 2.49 (3H, s), 1.48 (9H, m), 1.43 (3H, s).

### Preparation 87

### 5-{3-[3-(2,2-dimethyl-1,3-dioxen-5-yl)-2,3,4,5-tetrahydro-1H-3-benzazepin-7-yl]-1,2,4-oxadiazol-5-yl}-2-[(1-methylethyl)oxy]benzonitrile

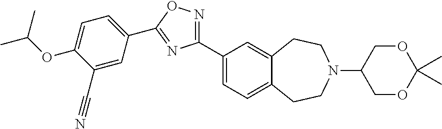

To a stirred suspension of 2-[(1-methylethyl)oxy]-5-[3-(2,3,4,5-tetrahydro-1H-3-benzazepin-7-yl)-1,2,4-oxadiazol-5-yl]benzonitrile hydrochloride (Preparation 43) (180 mg, 0.44 mmol) in DCM (4 ml) under nitrogen was added 2,2-dimethyl-1,3-dioxan-5-one (0.11 ml, 0.88 mmol) followed by sodium triacetoxyborohydride (290 mg, 1.3 mmol) portionwise. The mixture was stirred under nitrogen at room temperature for 23 h. Further 2,2-dimethyl-1,3-dioxan-5-one (0.053 ml, 0.44 mmol) followed by sodium triacetoxyborohydride (140 mg, 0.68 mmol) was added to the mixture which was then stirred for 18 h. Saturated aqueous sodium hydrogen carbonate solution (5 ml) was added to the mixture which was stirred vigorously for 15 min. The phases were separated and the aqueous phase was extracted with DCM (3×4 ml). The combined organic phases were dried (hydrophobic frit) and the solvent was evaporated under a stream of nitrogen to give 5-{3-[3-(2,2-dimethyl-1,3-dioxan-5-yl)-2,3,4,5-tetrahydro-1H-3-benzazepin-7-yl]-1,2,4-oxadiazol-5-yl}-2-[(1-methylethyl)oxy]benzonitrile as a pale yellow gum (286 mg). This material was used in the subsequent reaction without further purification (Example 68).

LCMS (Method formate): Retention time 1.09 min, MH⁺=489

### Preparation 88

### 2-[7-(5-{3-cyano-4-[(1-methylethyl)oxy]phenyl}-1,2,4-oxadiazol-3-yl)-1,2,4,5-tetrahydro-3H-3-benzazepin-3-yl]-N-((2S)-2-{[(1,1-dimethylethyl)(dimethyl)silyl]oxy}propyl)acetamide

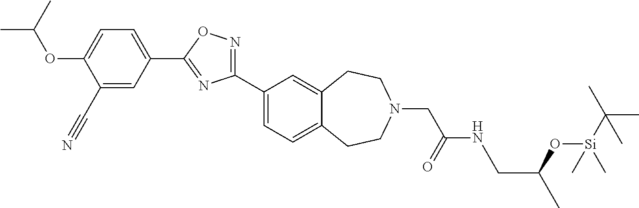

To [7-(5-{3-cyano-4-[(1-methylethyl)oxy]phenyl}-1,2,4-oxadiazol-3-yl)-1,2,4,5-tetrahydro-3H-3-benzazepin-3-yl]acetic acid (Preparation 89) (86 mg, 0.2 mmol) in DCM (3 ml) was added DMF (3 ml) at room temperature and most of the DCM was evaporated under a flow of nitrogen. EDC (46 mg, 0.24 mmol), HOBT (37 mg, 0.24 mmol), N-ethylmorpholine (0.076 ml, 0.60 mmol) were added to the solution and after 2 min ((25)-2-{[(1,1-dimethylethyl)(dimethyl)silyl]oxy]propyl)amine (57 mg, 0.30 mmol). The resulting solution was stirred for 72 h then concentrated in vacuo. The residue was dissolved in ethyl acetate and washed with saturated aqueous sodium hydrogen carbonate solution. The organic phase dried (MgSO₄) and concentrated in vacuo to give 2-[7-(5-{3-cyano-4-[(1-methylethyl)oxy]phenyl}-1,2,4-oxadiazol-3-yl)-1,2,4,5-tetrahydro-3H-3-benzazepin-3-yl}-N-((2S)-2-{[(1,1-dimethylethyl)(dimethyl)silyl]oxy}propyl)acetamide (110 mg). Used without further purification in the subsequent reaction (Example 75).

LCMS (Method HpH): Retention time 1.70 min, MH⁺=604

### Preparation 89

### [7-(5-{3-cyano-4-[(1-methylethyl)oxy]phenyl}-1,2,4-oxadiazol-3-yl)-1,2,4,5-tetrahydro-3H-3-benzazepin-3-yl]acetic acid

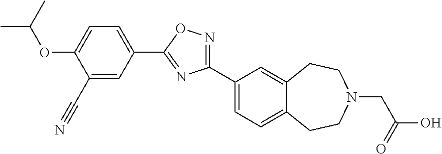

A suspension of methyl [7-(5-{3-cyano-4-[(1-methylethyl)oxy]phenyl}-1,2,4-oxadiazol-3-yl)-1,2,4,5-tetrahydro-3H-3-benzazepin-3-yl]acetate (Preparation 90) (940 mg, 2.1 mmol) in methanol at room temperature was treated with sodium hydroxide (2.1 ml, 4.2 mmol). THF (5 ml) was added and the mixture stirred for ˜24 h. Sodium hydroxide (2N, 1 ml) was added and stirring continued for 4 h. Acetic acid (0.48 ml, 8.4 mmol) was added to the reaction and most of the solvent evaporated. The residue was triturtated with methanol/water, the solid isolated by filtration and dried under vacuum overnight to give [7-(5-{3-cyano-4-[(1-methylethyl)oxy]phenyl}-1,2,4-oxadiazol-3-yl)-1,2,4,5-tetrahydro-3H-3-benzazepin-3-yl]acetic acid as a white solid (770 mg).

LCMS (Method formate): Retention time 0.90 min, MH⁺=433

### Preparation 89: Alternative Procedure [7-(5-{3-cyano-4-[(1-methylethyl)oxy]phenyl}-1,2,4-oxadiazol-3-yl)-1,2,4,5-tetrahydro-3H-3-benzazepin-3-yl) acetic acid trifluoroacetate

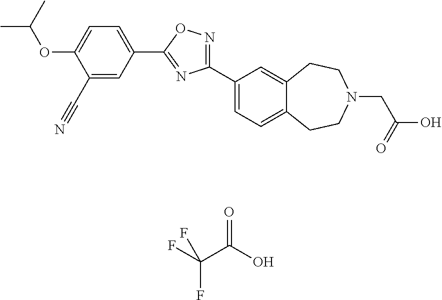

Trifluoroacetic acid (2 ml) was added to a stirred solution of 1,1-dimethylethyl [7-(5-{3-cyano-4-[(1-methylethyl)oxy]phenyl}-1,2,4-oxadiazol-3-yl)-1,2,4,5-tetrahydro-3H-3-benzazepin-3-yl]acetate (Preparation 186) (910 mg, 1.9 mmol) in DCM (10 ml). The reaction mixture was stirred at room temperature overnight. The solvent was evaporated. The residue was re-evaporated from toluene (x2). The residue was triturated with diethyl ether to give [7-(5-{3-cyano-4-[(1-methylethyl)oxy]phenyl}-1,2,4-oxadiazol-3-yl)-1,2,4,5-tetrahydro-3H-3-benzazepin-3-yl]acetic acid trifluoroacetate (1.0 g) as a colourless solid.

LCMS (Method formate): Retention time 0.94 min, MH⁺=433

### Preparation 90

### methyl [7-(5-{3-cyano-4-[(1-methylethyl)oxy]phenyl}-1,2,4-oxadiazol-3-yl)-1,2,4,5-tetrahydro-3H-3-benzazepin-3-yl]acetate

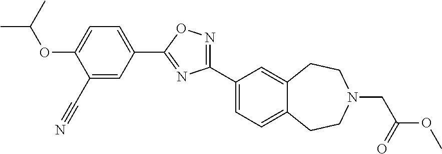

A suspension of 2-[(1-methylethyl)oxy]-5-[3-(2,3,4,5-tetrahydro-1H-3-benzazepin-7-yl)-1,2,4-oxadiazol-5-yl]benzonitrile hydrochloride (Preparation 43) (1.23 g, 3 mmol) and potassium carbonate (1.04 g, 7.5 mmol) in acetonitrile (15 ml) under nitrogen at room temperature was treated with methyl bromoacetate (0.29 ml, 3.2 mmol) and the resulting mixture was stirred at 60° C. After 2 h, further portions of potassium carbonate (210 mg) and methyl bromoacetate (0.058 ml) were added. Heating was continued for 30 min, the reaction cooled to room temperature, the solvent evaporated and the residue partitioned between saturated aqueous sodium hydrogen carbonate and ethyl acetate. The resulting emulsion was treated with toluene, diethyl ether, brine and DCM. Some organic phase was separated and the emulsion extracted with DCM (x5). The combined organics were dried (MgSO₄), concentrated in vacuo to give methyl [7-(5-{3-cyano-4-[(1-methylethyl)oxy]phenyl}-1,2,4-oxadiazol-3-yl)-1,2,4,5-tetrahydro-3H-3-benzazepin-3-yl]acetate as a white solid (940 mg).

LCMS (Method HpH): Retention time 1.36 min, MH⁺=447

### Preparation 91

### 2-[(1-methylethyl)oxy]-5-[3-(1,2,3,4-tetrahydro-5-isoquinolinyl)-1,2,4-oxadiazol-5-yl]benzonitrile trifluoroacetate

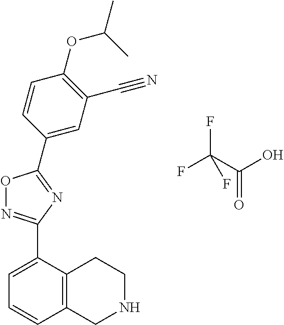

To a stirred solution of 1,1-dimethylethyl 5-(5-{3-cyano-4-[(1-methylethyl)oxy]phenyl}-1,2,4-oxadiazol-3-yl)-3,4-dihydro-2(1H)-isoquinolinecarboxylate (Preparation 92) (2 g, 4.3 mmol), at 0° C. in DCM (14 ml), was slowly added trifluoroacetic acid (7 ml). The solution was allowed to reach room temperature and stirred for 3 h. The solvent was evaporated under reduced pressure to give a brown oil which was re-evaporated with toluene (x2). The resultant orange oil was triturated with diethyl ether to give 2-[(1-methylethyl)oxy]-5-[3-(1,2,3,4-tetrahydro-5-isoquinolinyl)-1,2,4-oxadiazol-5-yl]benzonitrile trifluoroacetate as an off-white solid (2.3 g).

LCMS (Method formate): Retention time 0.87 min, MH⁺=402

### Preparation 92

### 1,1-dimethylethyl 5-(5-{3-cyano-4-[(1-methylethyl)oxy]phenyl}-1,2,4-oxadiazol-3-yl)-3,4-dihydro-2(1H)-isoquinolinecarboxylate

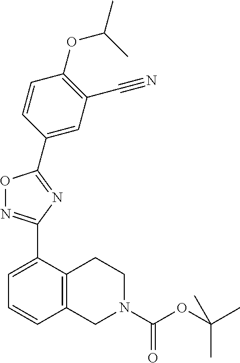

To a stirred solution of 1,1-dimethylethyl 5-[(hydroxyamino)(imino)methyl]-3,4-dihydro-2(1H)-isoquinolinecarboxylate (Preparation 93) (5.16 g, 17.7 mmol) in toluene (32 ml) and pyridine (16 ml) was added 3-cyano-4-[(1-methylethyl)oxy]benzoyl chloride (Preparation 169) (4.36 g, 18 mmol, WO2009080730/WO2008128951). The solution was refluxed at 120° C. for 3 h. The reaction mixture was cooled and evaporated under reduced pressure. The residue was suspended in ethyl acetate (100 ml) and washed with water (3×100 ml). The organic phase was dried (hydrophobic frit) and the solvent was evaporated under reduced pressure. The residual solid was dissolved in DCM and loaded onto a silica cartridge (100 g) and the cartridge eluted with an ethyl acetate/cyclohexane gradient (10-30%). The appropriate fractions were combined and evaporated under vacuum to give the required product 1,1-dimethylethyl 5-(5-{3-cyano-4-[(1-methylethyl)oxy]phenyl}-1,2,4-oxadiazol-3-yl)-3,4-dihydro-2(1H)-isoquinolinecarboxylate (2 g, 25%) as a white solid.

LCMS (Method formate): Retention time 1.50 min, MH⁺=466

### Preparation 93

### 1,1-dimethylethyl 5-[(hydroxyamino)(imino)methyl]-3,4-dihydro-2(1H)-isoquinolinecarboxylate

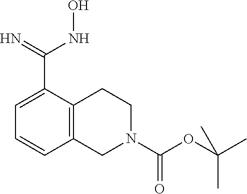

To a stirred solution of 1,1-dimethylethyl 5-cyano-3,4-dihydro-2(1H)-isoquinolinecarboxylate (Preparation 94) (5.93 g, 23 mmol) in ethanol (100 ml) was added, sodium hydrogen carbonate (9.64 g, 120 mmol) and hydroxylamine hydrochloride (7.98 g, 120 mmol). The solution was refluxed at 80° C. for 20 h, the reaction mixture filtered through a Celite™ column, and the solvent evaporated under reduced pressure. The resulting solid was triturated with water (50 ml), the water was decanted, toluene (50 ml) was added and evaporated off under reduced pressure (x2) to give 1,1-dimethylethyl 5-[(hydroxyamino)(imino)methyl]-3,4-dihydro-2(1H)-isoquinolinecarboxylate as a white solid (5.16 g).

LCMS (Method formate): Retention time 0.65 min, MH⁺=292

### Preparation 94

### 1,1-dimethylethyl 5-cyano-3,4-dihydro-2(1H)-isoquinolinecarboxylate

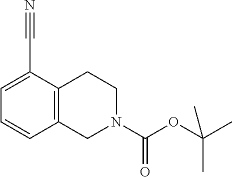

To a a stirred solution of 1,2,3,4-tetrahydro-5-isoquinolinecarbonitrile (2.95 g, 19 mmol, Astatach) in DCM (75 ml) was added di-tert-butyl dicarbonate (1.39 g, 22 mmol) slowly over 5 min. The reaction was stirred under nitrogen for 1 h and a further portion of di-tert butyl dicarbonate (3.09 g, 14 mmol) added. The reaction was left to stir at room temperature, under nitrogen for 2 h. The reaction mixture was evaporated under reduced pressure and the residual oil extracted using water (20 ml) and ethyl acetate (3×20 ml). The combined organic phases were dried (hydrophobic frit) and the solution evaporated under reduced pressure. The residue was dissolved in DCM and loaded onto a silica cartridge (100 g). The cartridge was eluted with an ethyl acetate/cyclohexane gradient (0-40% ethyl acetate). The appropriate fractions were combined and evaporated under vacuum to give the required product 1,1-dimethylethyl 5-cyano-3,4-dihydro-2(1H)-isoquinolinecarboxylate (5.93 g, 22.96 mmol, 123%) as a colourless oil.

LCMS (Method formate): Retention time 1.14 min, MH⁺=259 (weak)

### Preparation 95

### 1,1-dimethylethyl ((1S,2R)-1-{[7-(5-{5-cyano-6-[(1-methylethyl)oxy]-3-pyridinyl}-1,2,4-oxadiazol-3-yl)-1,2,4,5-tetrahydro-3H-3-benzazepin-3-yl]carbonyl}-2-hydroxypropyl)carbamate

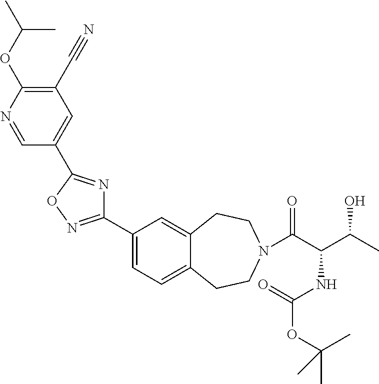

2-[(1-Methylethyl)oxy]-5-[3-(2,3,4,5-tetrahydro-1H-3-benzazepin-7-yl)-1,2,4-oxadiazol-5-yl]-3-pyridinecarbonitrile trifluoroacetate (Preparation 96) (50 mg, 0.10 mmol) was added to a solution of N-{[(1,1-dimethylethyl)oxy]carbonyl}-L-threonine (27 mg, 0.12 mmol), DIPEA (0.062 ml, 0.36 mmol) and HATU (58 mg, 0.15 mmol) in DMF (1 ml) and the mixture stirred for 4 h. The solvent was evaporated and the residue was dissolved in DCM and applied to a silica cartridge. The cartridge was eluted with an ethyl acetate/cyclohexane gradient (20-60%). The appropriate fractions were combined and evaporated in vacuo to give 1,1-dimethylethyl ((1S,2R)-1-{[7-(5-{5-cyano-6-[(1-methylethyl)oxy]-3-pyridinyl}-1,2,4-oxadiazol-3-yl)-1,2,4,5-tetrahydro-3H-3-benzazepin-3-yl}carbonyl]-2-hydroxypropyl)carbamate (58 mg) as a colourless gum.

LCMS (Method formate): Retention time 1.34 min, MH⁺=577

### Preparation 96

### 2-[(1-Methylethyl)oxy]-5-[3-(2,3,4,5-tetrahydro-1H-3-benzazepin-7-yl)-1,2,4-oxadiazol-5-yl]-3-pyridinecarbonitrile trifluoroacetate

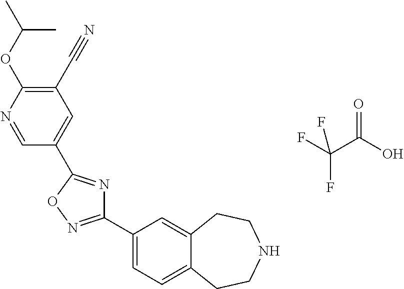

Trifluoroacetic acid (0.51 ml, 6.6 mmol) was added dropwise to a solution of 1,1-dimethylethyl 7-(5-{5-cyano-6-[(1-methylethyl)oxy]-3-pyridinyl}-1,2,4-oxadiazol-3-yl)-1,2,4,5-tetrahydro-3H-3-benzazepine-3-carboxylate (Preparation 97) (1.05 g, 2.2 mmol) in DCM (15 ml) at 0° C. under nitrogen. The mixture was allowed to warm to room temp and stirred for 16 h. Toluene (20 ml) was added and the solvent evaporated to give a colourless solid which was triturated under diethyl ether (20 ml) and the solid isolated by filtration to give 2-[(1-methylethyl)oxy]-5-[3-(2,3,4,5-tetrahydro-1H-3-benzazepin-7-yl)-1,2,4-oxadiazol-5-yl]-3-pyridinecarbonitrile trifluoroacetate as a colourless solid (1.03 g).

LCMS (Method formate): Retention time 0.97 min, MH⁺=376

### Preparation 97

### 1,1-dimethylethyl 7-(5-{5-cyano-6-[(1-methylethyl)oxy]-3-pyridinyl}-1,2,4-oxadiazol-3-yl)-1,2,4,5-tetrahydro-3H-3-benzazepine-3-carboxylate

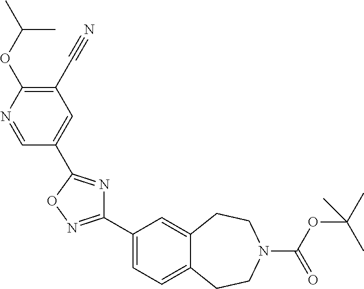

5-Cyano-6-[(1-methylethyl)oxy]-3-pyridinecarbonyl chloride (Preparation 98) (660 mg, 2.9 mmol) was added portionwise to a suspension of 1,1-dimethylethyl 7-[(hydroxyamino)(imino)methyl]-1,2,4,5-tetrahydro-3H-3-benzazepine-3-carboxylate (Preparation 41) (850 mg, 2.8 mmol) in toluene (10 ml) and pyridine (10 ml) at room temperature under nitrogen and the mixture stirred for 20 min. The mixture was then heated at 120° C. for 2 h and the mixture evaporated to dryness. The residue was dissolved in DCM, loaded onto a silica cartridge and the cartridge eluted with an ethyl acetate/cyclohexane gradient (0-50%). The appropriate fractions were combined and evaporated in vacuo to give 1,1-dimethylethyl 7-(5-{5-cyano-6-[(1-methylethyl)oxy}-3-pyridinyl]-1,2,4-oxadiazol-3-yl)-1,2,4,5-tetrahydro-3H-3-benzazepine-3-carboxylate (1.05 g) as a colourless foam.

LCMS (Method formate): Retention time 1.54 min, [2M+H]⁺=951

### Preparation 98

### 5-Cyano-6-[(1-methylethyl)oxy]-3-pyridinecarbonyl chloride

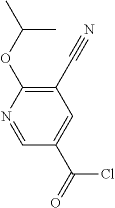

Oxalyl chloride (1.94 ml, 22 mmol) was added to a suspension of 5-cyano-6-[(1-methylethyl)oxy]-3-pyridinecarboxylic acid (3.04 g, 15 mmol) in DCM (50 ml), followed by DMF (0.01 ml, 0.15 mmol) and the mixture stirred for 3 h. The solution was evaporated and the residue azeotroped with toluene (3×10 ml) to give 5-cyano-6-[(1-methylethyl)oxy]-3-pyridinecarbonyl chloride as a pale yellow oil which solidified on standing under vacuum (3.5 g)

1H NMR (CDCl₃): 9.05 (1H, d), 8.53 (1H, d), 5.57 (1H, m), 1.46 (6H, d).

### Preparation 99

### 2-[7-(5-{3-cyano-4-[(1-methylethyl)oxy]phenyl}-1,2,4-oxadiazol-3-yl)-1,2,4,5-tetrahydro-3H-3-benzazepin-3-yl]-N-((1R)-2-{[(1,1-dimethylethyl)(dimethyl)silyl]oxy}-1-methylethyl)acetamide

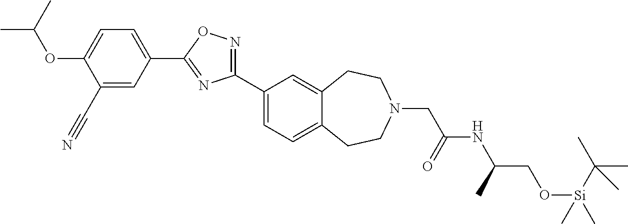

To a solution of [7-(5-{3-cyano-4-[(1-methylethyl)oxy]phenyl}-1,2,4-oxadiazol-3-yl)-1,2,4,5-tetrahydro-3H-3-benzazepin-3-yl]acetic acid (Preparation 89) (86 mg, 0.2 mmol) in DMF (3 ml) at room temperature was added EDC (46 mg, 0.24 mmol), N-ethylmorpholine (0.076 ml, 0.60 mmol) then HOBT (37 mg, 0.24 mmol). After 2 min, ((1R)-2-{[(1,1-dimethylethyl)(dimethyl)silyl]oxy}-1-methylethyl)amine (49 mg, 0.26 mmol) in DMF (1 ml) was added. The resulting mixture was stirred at room temperature for 16 h. The solvent was evaporated, the residue diluted with ethyl acetate and the mixture washed with saturated aqueous sodium hydrogen carbonate solution. The organic phase was dried (MgSO₄) and concentrated in vacuo to give 2-[7-(5-{3-cyano-4-[(1-methylethyl)oxy]phenyl}-1,2,4-oxadiazol-3-yl)-1,2,4,5-tetrahydro-3H-3-benzazepin-3-yl]-N-((1R)-2-{[(1,1-dimethylethyl)(dimethyl)silyl]oxy}-1-methylethyl)acetamide (160 mg, 128%) as a pale yellow oil, which was used in the next step without further purification (Example 78).

LCMS (Method HpH): Retention time 1.70 min, MH⁺=604

### Preparation 100

### 1,1-dimethylethyl ((1S,2R)-1-{[6-(5-{5-cyano-6-[(1-methylethyl)oxy]-3-pyridinyl}-1,2,4-oxadiazol-3-yl)-5-methyl-3,4-dihydro-2(1H)-isoquinolinyl]carbonyl}-2-hydroxypropyl)carbamate

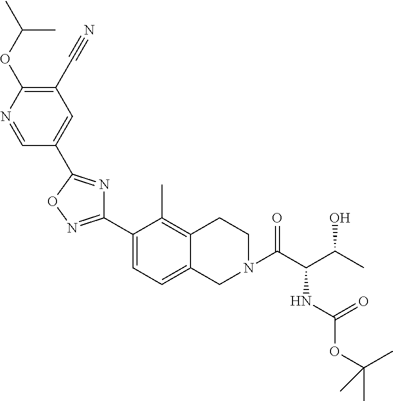

2-[(1-Methylethyl)oxy]-5-[3-(5-methyl-1,2,3,4-tetrahydro-6-isoquinolinyl)-1,2,4-oxadiazol-5-yl]-3-pyridinecarbonitrile trifluoroacetate (Preparation 3) (42 mg, 0.11 mmol) was added to a mixture of N-{[(1,1-dimethylethyl)oxy]carbonyl}-L-threonine (25 mg, 0.11 mmol), DIPEA (0.068 ml, 0.39 mmol) and HATU (64 mg, 0.17 mmol) in DMF (1 ml) and the mixture stirred for 4 h. The solvent was evaporated, the residue dissolved in DCM and loaded onto a silica cartridge. The cartridge was eluted with an ethyl acetate/cyclohexane gradient (20-60%). The appropriate fractions were combined and evaporated in vacuo to give 1,1-dimethylethyl ((1S,2R)-1-{[6-(5-{5-cyano-6-[(1-methylethyl)oxy]-3-pyridinyl}-1,2,4-oxadiazol-3-yl)-5-methyl-3,4-dihydro-2(1H)-isoquinolinyl]carbonyl}-2-hydroxypropyl)carbamate (29 mg) as a colourless gum.

LCMS (Method formate): Retention time 1.33 min, MH⁺=577

### Preparation 101

### 2-[7-(5-{3-cyano-4-[(1-methylethyl)oxy]phenyl}-1,2,4-oxadiazol-3-yl)-1,2,4,5-tetrahydro-3H-3-benzazepin-3-yl]-N-((1S)-2-{[(1,1-dimethylethyl)(dimethyl)silyl]oxy}-1-methylethyl)acetamide

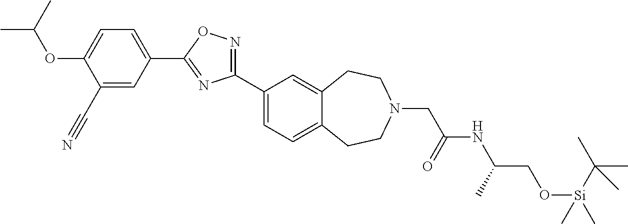

To a solution of [7-(5-{3-cyano-4-[(1-methylethyl)oxy]phenyl}-1,2,4-oxadiazol-3-yl)-1,2,4,5-tetrahydro-3H-3-benzazepin-3-yl]acetic acid (Preparation 89) (86 mg, 0.2 mmol) in DMF (3 ml) at room temperature was added EDC (46 mg, 0.24 mmol), N-ethylmorpholine (0.076 ml, 0.60 mmol) then HOBT (37 mg, 0.24 mmol). After 2 min, ((1S)-2-{[(1,1-dimethylethyl)(dimethyl)silyl]oxy}-1-methylethyl)amine (49 mg, 0.26 mmol) in DMF (1 ml) was added. The resulting mixture was stirred at room temperature for 16 h. The solvent was evaporated, the residue diluted with ethyl acetate and the mixture washed with saturated aqueous sodium hydrogen carbonate solution. The organic phase was dried (MgSO₄) and concentrated in vacuo to give 2-[7-(5-{3-cyano-4-[(1-methylethyl)oxy]phenyl}-1,2,4-oxadiazol-3-yl)-1,2,4,5-tetrahydro-3H-3-benzazepin-3-yl]-N-((1S)-2-{[(1,1-dimethylethyl)(dimethyl)silyl]oxy}-1-methylethyl)acetamide (120 mg, 99%) as a pale yellow oil which was used in the next step without further purification (Example 80).

LCMS (Method HpH): Retention time 1.70 min, MH⁺=604

### Preparation 102

### 5-{3-[3-(bromoacetyl)-2,3,4,5-tetrahydro-1H-3-benzazepin-7-yl]-1,2,4-oxadiazol-5-yl}-2-[(1-methylethyl)oxy]benzonitrile

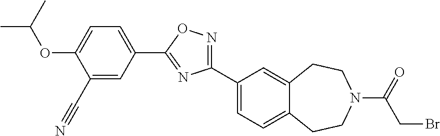

To a suspension of 2-[(1-methylethyl)oxy]-5-[3-(2,3,4,5-tetrahydro-1H-3-benzazepin-7-yl)-1,2,4-oxadiazol-5-yl]benzonitrile hydrochloride (Preparation 43) (820 mg, 2 mmol) in DCM (15 ml) at 0° C. under nitrogen was added DIPEA (0.87 ml, 5.0 mmol). Bromoacetyl bromide (0.18 ml, 2.1 mmol) in DCM (5 ml) was added dropwise and the mixture was then stirred at 0° C. for 40 min. DIPEA (0.5 ml) was added, followed by a solution of bromoacetyl bromide (0.1 ml) in DCM (2 ml) and stirring continued for 10 min. Isopropanol (0.3 ml) was added and the mixture washed with hydrochloric acid (2N) and saturated sodium hydrogen carbonate. The sodium hydrogen carbonate washes were extracted (x2), the combined organic washed with water, dried (hydrophobic frit) then concentrated in vacuo to give 5-{3-[3-(bromoacetyl)-2,3,4,5-tetrahydro-1H-3-benzazepin-7-yl]-1,2,4-oxadiazol-5-yl}-2-[(1-methylethyl)oxy]benzonitrile as a brown foam (1.0 g, 2.011 mmol, 101%), which was used in next step without further purification (Example 86).

LCMS (Method HpH): Retention time 1.31 min, MH⁺=495/497

### Preparation 103

### 1,1-dimethylethyl {2-[5-(5-{3-cyano-4-[(1-methylethyl)oxy]phenyl}-1,2,4-oxadiazol-3-yl)-3,4-dihydro-2(1H)-isoquinolinyl]-2-oxoethyl}carbamate

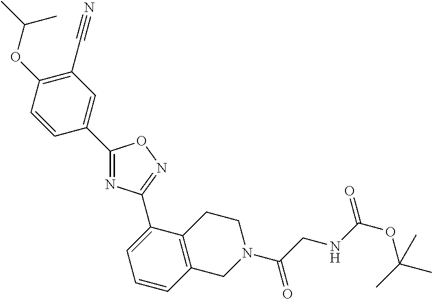

A mixture of N-Bocglycine (53 mg, 0.3 mmol), N-ethylmorpholine (70 mg, 77 μl, 0.6 mmol), hydroxybenzotriazole hydrate (56 mg, 0.36 mmol), EDC (70 mg, 0.36 mmol) and 2-[(1-methylethyl)oxy]-5-[3-(1,2,3,4-tetrahydro-5-isoquinolinyl)-1,2,4-oxadiazol-5-yl]benzonitrile trifluoroacetate (Preparation 91) (170 mg, 0.36 mmol) in DMF (3 ml) was stirred at room temperature overnight. The reaction mixture was partitioned between ethyl acetate (20 ml) and saturated sodium hydrogen carbonate (20 ml). The organic phase was washed with hydrochloric acid (1M), water and brine. Dried and evaporated. The residue was chromatographed (methanol/DCM, 0-4%) to give 1,1-dimethylethyl {2-[5-(5-{3-cyano-4-[(1-methylethyl)oxy]phenyl}-1,2,4-oxadiazol-3-yl)-3,4-dihydro-2(1H)-isoquinolinyl]-2-oxoethyl}carbamate as a colourless solid (150 mg)

LCMS (Method formate): Retention time 1.31 min, MH⁺=518

### Preparation 104

### 1,1-dimethylethyl {3-[5-(5-{3-cyano-4-[(1-methylethyl)oxy]phenyl}-1,2,4-oxadiazol-3-yl)-3,4-dihydro-2(1H)-isoquinolinyl]-3-oxopropyl}carbamate

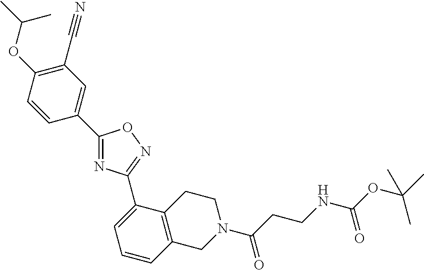

A mixture of N-Boc-β-alanine (57 mg, 0.3 mmol), N-ethylmorpholine (77 μl, 0.6 mmol), hydroxybenzotriazole hydrate (56 mg, 0.36 mmol), EDC (70 mg, 0.36 mmol) and 2-[(1-methylethyl)oxy]-5-[3-(1,2,3,4-tetrahydro-5-isoquinolinyl)-1,2,4-oxadiazol-5-yl]benzonitrile trifluoroacetate (Preparation 91) (170 mg, 0.36 mmol) in DMF (3 ml) was stirred at room temperature overnight. The reaction mixture was partitioned between ethyl acetate (20 ml) and saturated sodium hydrogen carbonate (20 ml). The organic phase was washed with hydrochloric acid (1M), water and brine. Dried and evaporated. The residue was chromatographed (methanol/DCM, 0-4%) to give 1,1-dimethylethyl {3-[5-(5-{3-cyano-4-[(1-methylethyl)oxy]phenyl}-1,2,4-oxadiazol-3-yl)-3,4-dihydro-2(1H)-isoquinolinyl]-3-oxopropyl}carbamate as a colourless oil (150 mg).

LCMS (Method formate): Retention time 1.31 min, MH⁺=532

### Preparation 105

### 1,1-dimethylethyl [(1S)-2-[5-(5-{3-cyano-4-[(1-methylethyl)oxy]phenyl}-1,2,4-oxadiazol-3-yl)-3,4-dihydro-2(1H)-isoquinolinyl]-1-(hydroxymethyl)-2-oxoethyl]carbamate

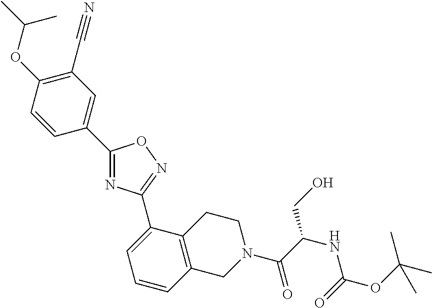

A mixture of N-Boc-L-serine (62 mg, 0.3 mmol), N-ethylmorpholine (77 μl, 0.6 mmol), hydroxybenzotriazole hydrate (56 mg, 0.36 mmol), EDC (70 mg, 0.36 mmol) and 2-[(1-methylethyl)oxy]-5-[3-(1,2,3,4-tetrahydro-5-isoquinolinyl)-1,2,4-oxadiazol-5-yl]benzonitrile trifluoroacetate (Preparation 91) (170 mg, 0.36 mmol) in DMF (3 ml) was stirred at room temperature overnight. The reaction mixture was partitioned between ethyl acetate (20 ml) and saturated sodium hydrogen carbonate (20 ml). The organic phase was washed with hydrochloric acid (1M), water and brine. Dried and evaporated. The residue was chromatographed (methanol/DCM, 0-4%) to give 1,1-dimethylethyl [(1S)-2-[5-(5-{3-cyano-4-[(1-methylethyl)oxy]phenyl}-1,2,4-oxadiazol-3-yl)-3,4-dihydro-2(1H)-isoquinolinyl]-1-(hydroxymethyl)-2-oxoethyl]carbamate as a colourless solid (150 mg).

LCMS (Method formate): Retention time 1.23 min, MH⁺=548

### Preparation 106

### 1,1-dimethylethyl [(1R)-2-[5-(5-{3-cyano-4-[(1-methylethyl)oxy]phenyl}-1,2,4-oxadiazol-3-yl)-3,4-dihydro-2(1H)-isoquinolinyl]-1-(hydroxymethyl)-2-oxoethyl]carbamate

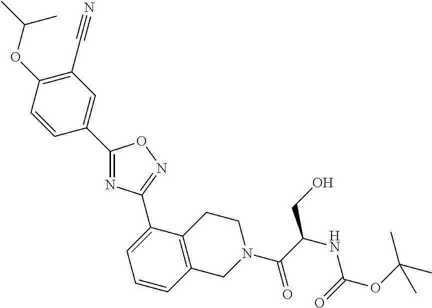

A mixture of N-Boc-D-serine (62 mg, 0.3 mmol), N-ethylmorpholine (77 μl, 0.6 mmol), hydroxybenzotriazole hydrate (56 mg, 0.36 mmol), EDC (70 mg, 0.36 mmol) and 2-[(1-methylethyl)oxy]-5-[3-(1,2,3,4-tetrahydro-5-isoquinolinyl)-1,2,4-oxadiazol-5-yl]benzonitrile trifluoroacetate (Preparation 91) (170 mg, 0.36 mmol) in DMF (3 ml) was stirred at room temperature overnight. The reaction mixture was partitioned between ethyl acetate (20 ml) and saturated sodium hydrogen carbonate (20 ml). The organic phase was washed with hydrochloric acid (1M), water and brine. Dried and evaporated. The residue was chromatographed (methanol/DCM, 0-4%) to give 1,1-dimethylethyl [(1R)-2-[5-(5-{3-cyano-4-[(1-methylethyl)oxy]phenyl}-1,2,4-oxadiazol-3-yl)-3,4-dihydro-2(1H)isoquinolinyl]-1-(hydroxymethyl)-2-oxoethyl]carbamate as a colourless solid (139 mg).

LCMS (Method formate): Retention time 1.23 min, MH⁺=548

### Preparation 107

### 5-(3-{3-[2-(3-{[(1,1-dimethylethyl)(dimethyl)silyl]oxy}-1-azetidinyl)-2-oxoethyl]-2,3,4,5-tetrahydro-1H-3-benzazepin-7-yl}-1,2,4-oxadiazol-5-yl)-2-[(1-methylethyl)oxy]benzonitrile

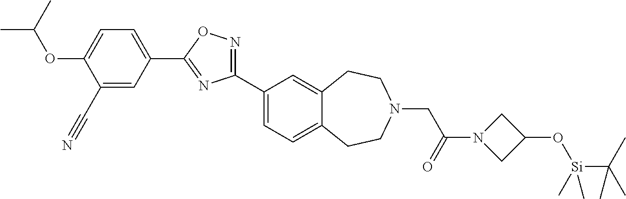

A solution of [7-(5-{3-cyano-4-[(1-methylethyl)oxy]phenyl}-1,2,4-oxadiazol-3-yl)-1,2,4,5-tetrahydro-3H-3-benzazepin-3-yl]acetic acid (Preparation 89) (86 mg, 0.2 mmol) in DMF (3 ml) at room temperature was treated with EDC (46 mg, 0.24 mmol), N-ethylmorpholine (0.076 ml, 0.60 mmol) then HOBT (37 mg, 0.24 mmol) and after 2 min with 3-{[(1,1-dimethylethyl)(dimethyl)silyl]oxy}azetidine (56 mg, 0.30 mmol). The resulting mixture was stirred for 16 h at room temperature then concentrated in vacuo. The residue was dissolved in ethyl acetate and the mixture washed with saturated sodium hydrogen carbonate (x2) then dried (MgSO₄) and concentrated in vacuo to give 5-(3-{3-[2-(3-{[(1,1-dimethylethyl)(dimethyl)silyl]oxy}-1-azetidinyl)-2-oxoethyl]-2,3,4,5-tetrahydro-1H-3-benzazepin-7-yl}-1,2,4-oxadiazol-5-yl)-2-[(1-methylethyl)oxy]benzonitrile (120 mg, 100%) as a pale yellow oil. The mixture used without further purification in the subsequent reaction (Example 93).

LCMS (Method HpH): Retention time 1.63 min, MH⁺=602

### Preparation 108

### 2-(3-fluoro-1-azetidinyl)-5-[3-(2,3,4,5-tetrahydro-1H-3-benzazepin-7-yl)-1,2,4-oxadiazol-5-yl]benzonitrile

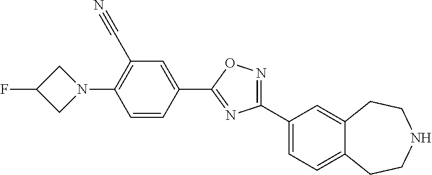

1,1-Dimethylethyl 7-{5-[3-cyano-4-(3-fluoro-1-azetidinyl)phenyl]-1,2,4-oxadiazol-3-yl]-1,2,4,5-tetrahydro-3H-3-benzazepine-3-carboxylate (Preparation 109) (280 mg, 0.56 mmol) was dissovled in DCM (8 ml), and trifluoroacetic acid (2 ml, 26 mmol) was added. The reaction mixture was stirred at room temperature for 1.5 h and concentrated in vacuo. The residual solid was dissolved in DCM and filtered through an aminopropyl SPE (5 g) washing the SPE with methanol in DCM (10%). The appropriate fractions were combined and evaporated in vacuo to give 2-(3-fluoro-1-azetidinyl)-5-[3-(2,3,4,5-tetrahydro-1H-3-benzazepin-7-yl)-1,2,4-oxadiazol-5-yl]benzonitrile as a white solid (220 mg).

LCMS (Method formate): Retention time 0.82 min, MH⁺=390

### Preparation 109

### 1,1-Dimethylethyl 7-{5-[3-cyano-4-(3-fluoro-1-azetidi nyl)phenyl]-1,2,4-oxadiazol-3-yl}-1,2,4,5-tetrahydro-3H-3-benzazepine-3-carboxylate

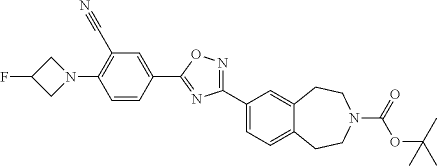

To 3-fluoroazetidine (350 mg, 3.1 mmol) in DMF (8 ml) at room temperature was added sodium hydride (60% in mineral oil, 170 mg, 4.1 mmol) and the reaction stirred for 10 min. 1,1-Dimethylethyl 7-[5-(3-cyano-4-fluorophenyl)-1,2,4-oxadiazol-3-yl]-1,2,4,5-tetrahydro-3H-3-benzazepine-3-carboxylate (Preparation 68) (450 mg, 1.0 mmol) was added and the reaction was stirred overnight. The reaction mixture was partitioned between ethyl acetate (2×50 ml) and saturated aqueous sodium hydrogen carbonate solution (50 ml). The organic phases were combined, dried (hydrophobic frit) and reduced to dryness in vacuo. The sample dissolved in DCM, loaded onto a silica cartridge (100 g) and the cartridge eluted with an ethyl acetate/cyclohexane gradient (0-30%) followed by continued elution with ethyl acetate/cyclohexane (30%). The appropriate fractions were combined and reduced to dryness under a stream of nitrogen to give 1,1-dimethylethyl 7-{5-[3-cyano-4-(3-fluoro-1-azetidinyl)phenyl}-1,2,4-oxadiazol-3-yl]-1,2,4,5-tetrahydro-3H-3-benzazepine-3-carboxylate as an off-white solid (280 mg).

LCMS (Method formate): Retention time 1.43 min, [M-tBu+MeCN]H⁺=475

### Preparation 110

### 5-{3-[3-(2,2-dimethyl-1,3-dioxan-5-yl)-2,3,4,5-tetrahydro-1H-3-benzazepin-7-yl]-1,2,4-oxadiazol-5-yl]-2-[(1-methylethyl)oxy]-3-pyridinecarbonitrile

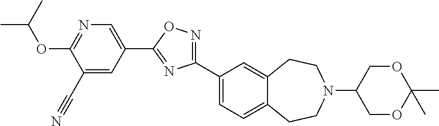

To a stirred suspension of 2-[(1-methylethyl)oxy]-5-[3-(2,3,4,5-tetrahydro-1H-3-benzazepin-7-yl)-1,2,4-oxadiazol-5-yl]-3-pyridinecarbonitrile trifluoroacetate (Preparation 96) (250 mg, 0.5 mmol) in DCM (8 ml) under nitrogen was added 2,2-dimethyl-1,3-dioxan-5-one (0.18 ml, 1.5 mmol) followed by sodium triacetoxyborohydride (490 mg, 2.3 mmol). The mixture was stirred under nitrogen at room temperature for 70 h. Saturated aqueous sodium hydrogen carbonate solution (5 ml) was added to the mixture which was then stirred vigorously for 15 min. The phases were separated and the aqueous phase was extracted with DCM (3×4 ml). The combined organic phases were dried (hydrophobic frit) and the solvent was evaporated under a stream of nitrogen to give 5-{3-[3-(2,2-dimethyl-1,3-dioxan-5-yl)-2,3,4,5-tetrahydro-1H-3-benzazepin-7-yl]-1,2,4-oxadiazol-5-yl]-2-[(1-methylethyl)oxy}-3-pyridinecarbonitrile as a cream solid (230 mg). Used in the subsequent reaction without further purification (Example 95).

LCMS (Method formate): Retention time 1.05 min, MH⁺=490

### Preparation 111

### 5-{3-[3-(2-Aminoethyl)-2,3,4,5-tetrahydro-1H-3-benzazepin-7-yl]-1,2,4-oxadiazol-5-yl}-2-[(1-methylethyl)oxy]benzonitrile hydrochloride

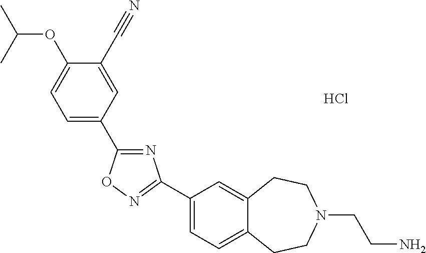

1,1-Dimethylethyl {2-[7-(5-{3-cyano-4-[(1-methylethyl)oxy]phenyl}-1,2,4-oxadiazol-3-yl)-1,2,4,5-tetrahydro-3H-3-benzazepin-3-yl]ethyl}carbamate (Preparation 112) (1.51 g, 2.9 mmol) was dissolved in hydrogen chloride in 1,4-dioxane (4M, 7.3 ml, 29 mmol) and stirred at room temperature for 30 min. Ether (10 ml) was added and the solution stirred for 10 min. The precipitate was isolated by filtration and washed with diethyl ether to give 5-{3-[3-(2-aminoethyl)-2,3,4,5-tetrahydro-1H-3-benzazepin-7-yl]-1,2,4-oxadiazol-5-yl}-2-[(1-methylethyl)oxy]benzonitrile hydrochloride as white solid (1.48 g).

LCMS (Method formate): Retention time 0.73 min, MH⁺=418

### Preparation 112

### 1,1-Dimethylethyl {2-[7-(5-{3-cyano-4-[(1-methylethyl)oxy]phenyl}-1,2,4-oxadiazol-3-yl)-1,2,4,5-tetrahydro-3H-3-benzazepin-3-yl]ethyl}carbamate

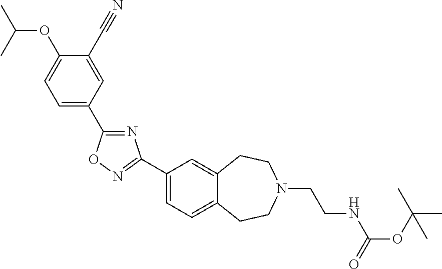

2-[(1-Methylethyl)oxy]-5-[3-(2,3,4,5-tetrahydro-1H-3-benzazepin-7-yl)-1,2,4-oxadiazol-5-yl]benzonitrile hydrochloride (Preparation 43) (1.23 g, 3.0 mmol), 1,1-dimethylethyl (2-bromoethyl)carbamate (1.01 g, 4.5 mmol) and potassium carbonate (1.24 g, 9.0 mmol) were dissolved in DMF (15 ml) and heated to 45° C. for 4 h. The reaction was left to cool. Water (50 ml) was added and the mixture was extracted with ethyl acetate (3×15 ml). The organic extracts were dried (hydrophobic frit) and the solvent evaporated under vacuum. The resulting liquid was purified by flash chromatography (isopropanol/DCM, 0-20%) to give 1,1-dimethylethyl {2-[7-(5-{3-cyano-4-[(1-methylethyl)oxy]phenyl}-1,2,4-oxadiazol-3-yl)-1,2,4,5-tetrahydro-3H-3-benzazepin-3-yl]ethyl}carbamate as a pale yellow oil (1.51 g).

LCMS (Method formate): Retention time 1.00 min, MH⁺=518

### Preparation 113

### 5-{3-[3-(3-aminopropyl)-2,3,4,5-tetrahydro-1H-3-benzazepin-7-yl]-1,2,4-oxadiazol-5-yl}-2-[(1-methylethyl)oxy]benzonitrile hydrochloride

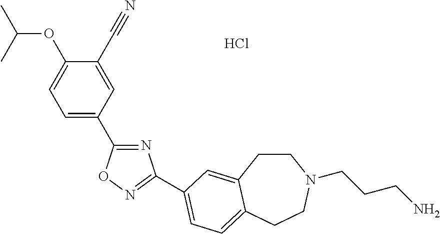

1,1-Dimethylethyl {3-[7-(5-{3-cyano-4-[(1-methylethyl)oxy]phenyl}-1,2,4-oxadiazol-3-yl)-1,2,4,5-tetrahydro-3H-3-benzazepin-3-yl]propyl}carbamate (Preparation 114) (1.63 g, 3.1 mmol) was dissolved in a mixture of hydrogen chloride in 1,4-dioxane (4M, 3.83 ml, 15 mmol) and 1,4-dioxane (10 ml) and left to stir at room temperature for 2 h. Ether (20 ml) was added and the solution was left to stir for another 15 min. A precipitate was formed and isolated by filtration to give 5-{3-[3-(3-aminopropyl)-2,3,4,5-tetrahydro-1H-3-benzazepin-7-yl]-1,2,4-oxadiazol-5-yl}-2-[(1-methylethyl)oxy]benzonitrile hydrochloride as a cream solid (1.3 g).

### Preparation 114

### 1,1-dimethylethyl {3-[7-(5-{3-cyano-4-[(1-methylethyl)oxy]phenyl}-1,2,4-oxadiazol-3-yl)-1,2,4,5-tetrahydro-3H-3-benzazepin-3-yl]propyl}carbamate

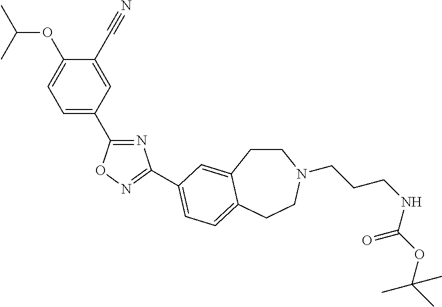

4-[7-(5-{3-cyano-4-[(1-methylethyl)oxy]phenyl}-1,2,4-oxadiazol-3-yl)-1,2,4,5-tetrahydro-3H-3-benzazepin-3-yl]butanoic acid sodium salt (Preparation 45) ((2.6 g, 5.4 mmol), diphenylphosphoryl azide (1.74 ml, 8.1 mmol) and triethylamine (2.25 ml, 16 mmol) were dissolved in a mixture of toluene (15 ml) and tert-butanol (10 ml). The mixture was heated to 80° C. for 2 h. The volatiles were evaporated under vacuum. Water (25 ml) was added to the residue and the mixture was extracted with ethyl acetate (3×15 ml). The combined organic phases were dried (hydrophobic frit) and the solvent evaporated under vacuum. The product was purified by flash chromatography (ethanol/DCM, 0-10%), which gave 1,1-dimethylethyl {3-[7-(5-{3-cyano-4-[(1-methylethyl)oxy]phenyl}-1,2,4-oxadiazol-3-yl)-1,2,4,5-tetrahydro-3H-3-benzazepin-3-yl]propyl}carbamate as an orange oil (1.63 g).

LCMS (Method HpH): Retention time 1.47 min, MH⁺=532

### Preparation 115

### 1,1-dimethylethyl [2-(7-{5-[5-cyano-6-(propyloxy)-3-pyridinyl]-1,2,4-oxadiazol-3-yl}-1,2,4,5-tetrahydro-3H-3-benzazepin-3-yl)-2-oxoethyl]carbamate

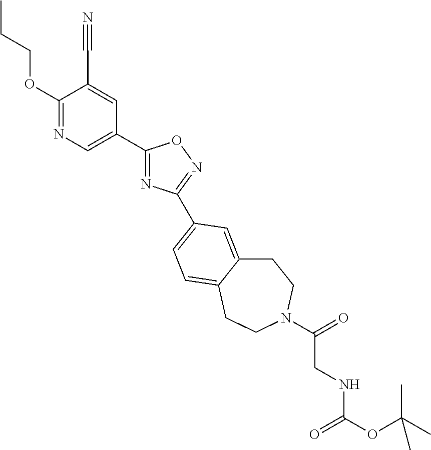

To N-BOC glycinamide (56 mg, 0.32 mmol) and HATU (150 mg, 0.40 mmol) in DMF (3 ml) was added DIPEA (0.14 ml, 0.80 mmol) and the reaction mixture stirred at room temperature for 10 min. 2-(Propyloxy)-5-[3-(2,3,4,5-tetrahydro-1H-3-benzazepin-7-yl)-1,2,4-oxadiazol-5-yl]-3-pyridinecarbonitrile (Preparation 116) (100 mg, 0.27 mmol) was added and the reaction mixture stirred at room temperature for a further 3 h. The solvent was evaporated under a stream of nitrogen. The residue was partitioned between DCM (3×10 ml) and water (10 ml). The organics were combined, dried (hydrophobic frit) and evaporated under a stream of nitrogen to give 1,1-dimethylethyl [2-(7-{5-[5-cyano-6-(propyloxy)-3-pyridinyl]-1,2,4-oxadiazol-3-yl}-1,2,4,5-tetrahydro-3H-3-benzazepin-3-yl)-2-oxoethyl]carbamate (310 mg) which was used without further purification in the subsequent reaction (Example 107).

LCMS (Method formate): Retention time 1.34 min, MH⁺=533

### Preparation 116

### 2-(Propyloxy)-5-[3-(2,3,4,5-tetrahydro-1H-3-benzazepin-7-yl)-1,2,4-oxadiazol-5-yl]-3-pyridinecarbonitrile

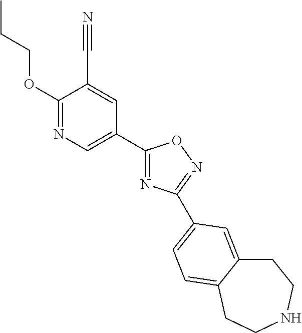

To 1,1-dimethylethyl 7-{5-[5-cyano-6-(propyloxy)-3-pyridinyl]-1,2,4-oxadiazol-3-yl}-1,2,4,5-tetrahydro-3H-3-benzazepine-3-carboxylate (Preparation 117) (1.1 g, 2.3 mmol) in DCM (18 ml) was added trifluoroacetic acid (2 ml, 26 mmol) and the reaction mixture stirred at room temperature for 1 h 30 min. The reaction mixture was applied directly to an aminopropyl SPE (50 g) and the SPE eluted with methanol in DCM (10%). The approproate fractions were combined and the volatiles evaporated to give 2-(propyloxy)-5-[3-(2,3,4,5-tetrahydro-1H-3-benzazepin-7-yl)-1,2,4-oxadiazol-5-yl]-3-pyridinecarbonitrile (930 mg).

LCMS (Method formate): Retention time 0.92 min, MH⁺=376

### Preparation 117

### 1,1-dimethylethyl 7-{5-[5-cyano-6-(propyloxy)-3-pyridinyl]-1,2,4-oxadiazol-3-yl}-1,2,4,5-tetrahydro-3H-3-benzazepine-3-carboxylate

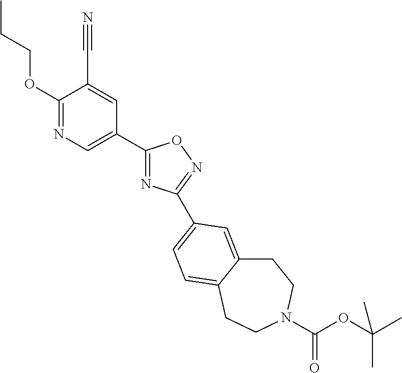

To 5-cyano-6-(propyloxy)-3-pyridinecarboxylic acid (Preparation 118) (1.15 g, 5.6 mmol) and HATU (2.38 g, 6.3 mmol) in DMF (10 ml) was added DIPEA (2.92 ml, 17 mmol) and the reaction mixture stirred at room temperature for 5 min. 1,1-Dimethylethyl 7-[(hydroxyamino)(imino)methyl]-1,2,4,5-tetrahydro-3H-3-benzazepine-3-carboxylate (Preparation 41) (2.04 g, 6.7 mmol) was added and the reaction mixture stirred at room temperature for 30 min. The reaction mixture was then heated at 100° C. for 1 h, and then allowed to cool overnight. The solvent was evaporated in vacuo, and the residue partitioned between DCM (3×100 ml) and water (100 ml). The organics were combined, dried (hydrophobic frit) and evaporated in vacuo. The sample was dissolved in methanol/DCM (5%) applied to silica cartridges (2×100 g), the columns dried in vacuo and the columns eluted with an ethyl acetate/cyclohexane gradient (0-25%) followed by continued elution with ethyl acetate/cyclohexane (25%). The appropriate fractions were combined and evaporated in vacuo to give 1,1-dimethylethyl 7-{5-[5-cyano-6-(propyloxy)-3-pyridinyl]-1,2,4-oxadiazol-3-yl}-1,2,4,5-tetrahydro-3H-3-benzazepine-3-carboxylate (1.1 g).

LCMS (Method formate): Retention time 1.53 min, MH⁺ not seen

### Preparation 118

### 5-cyano-6-(propyloxy)-3-pyridinecarboxylic acid

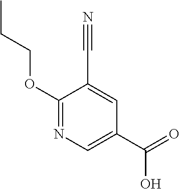

To methyl 5-cyano-6-(propyloxy)-3-pyridinecarboxylate (Preparation 119) (1.2 g, 5.5 mmol) in methanol (20 ml) was added lithium hydroxide (0.65 g, 27 mmol) in water (6 ml) and the reaction mixture stirred at room temperature for 2 h. The solvent was evaporated and water (50 ml) added. The mixture was acidified to pH1 using aqueous hydrochloric acid (2M) and extracted with DCM (3×50 ml). The organics were combined, dried (hydrophobic frit) and evaporated in vacuo to give 5-cyano-6-(propyloxy)-3-pyridinecarboxylic acid (1.15 g).

LCMS (Method formate): Retention time 0.87 min, [M−H]=205

### Preparation 119

### methyl 5-cyano-6-(propyloxy)-3-pyridinecarboxylate

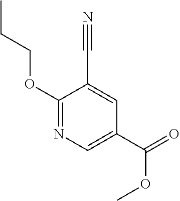

To methyl 5-cyano-6-oxo-1,6-dihydro-3-pyridinecarboxylate (2 g, 11 mmol, Enamine) in chloroform (100 ml) was added silver carbonate (6.19 g, 22 mmol) and 1-iodopropane (4.38 ml, 45 mmol) and the reaction mixture stirred at 100° C. overnight. The reaction mixture was filtered, the residue washed with chloroform, and the combined filtrate and washings reduced to dryness in vacuo. The residue was dissolved in DCM and loaded onto a silica cartridge (100 g), which was eluted with an ethyl acetate/cyclohexane gradient (0-50% ethyl acetate) followed by continued elution with ethyl acetate/cyclohexane (50%). The appropriate fractions were combined and evaporated in vacuo to give methyl 5-cyano-6-(propyloxy)-3-pyridinecarboxylate (1.2 g).

LCMS (Method formate): Retention time 1.07 min, MH⁺=220

### Preparation 120

### 2-[(1-methylethyl)amino]-5-[3-(2,3,4,5-tetrahydro-1H-3-benzazepin-7-yl)-1,2,4-oxadiazol-5-yl]-3-pyridinecarbonitrile hydrochloride

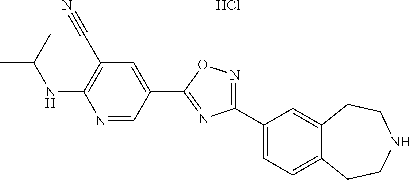

1,1-Dimethylethyl 7-(5-{5-cyano-6-[(1-methylethyl)amino]-3-pyridinyl}-1,2,4-oxadiazol-3-yl)-1,2,4,5-tetrahydro-3H-3-benzazepine-3-carboxylate (Preparation 121) (1.9 g, 4 mmol) was suspended in 1,4-dioxane (10 ml) and treated with hydrogen chloride in 1,4-dioxane (4M, 10 ml). The reaction mixture was stirred at room temperature for 2 h. Hydrogen chloride in 1,4-dioxane (4M, 10 ml) was added and the mixture stirred at room temperature for 6 h. The reaction was concentrated to ˜15 ml and diethyl ether (75 ml) added. The solid was isolated by filtration, washed with diethyl ether and dried to give 2-[(1-methylethyl)amino]-5-[3-(2,3,4,5-tetrahydro-1H-3-benzazepin-7-yl)-1,2,4-oxadiazol-5-yl]-3-pyridinecarbonitrile hydrochloride as a colourless solid (1.37 g).

LCMS (Method formate): Retention time 0.83 min, MH⁺=375

### Preparation 120: Alternative Procedure

### 2-[(1-methylethyl)amino]-5-[3-(2,3,4,5-tetrahydro-1H-3-benzazepin-7-yl)-1,2,4-oxadiazol-5-yl]-3-pyridinecarbonitrile

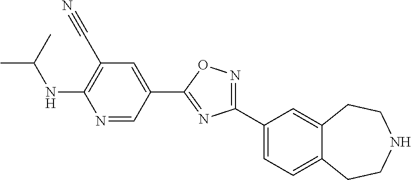

Trifluoroacetic acid (1 ml, 13 mmol) was added to a solution of 1,1-dimethylethyl 7-(5-{5-cyano-6-[(1-methylethyl)amino]-3-pyridinyl}-1,2,4-oxadiazol-3-yl)-1,2,4,5-tetrahydro-3H-3-benzazepine-3-carboxylate (Preparation 121) (1.24 g, 2.6 mmol) in DCM (10 ml) stirred at 0° C. (ice-water bath) and the mixture stirred for 10 mins before removing the ice-water bath. The reaction was stirred at room temperature for ˜24 h. DCM (10 ml) was added to the mixture and stirring continued overnight. Trifluoroacetic acid (0.5 ml, 6.5 mmol) was added, and the mixture stirred for 2 h, and the reaction mixture was concentrated in vacuo. The residue was dissolved in DCM, loaded onto an aminopropyl SPE (20 g) and the SPE eluted using methanol in DCM (10%). The appropriate fractions were combined and evaporated in vacuo. The residue was dissolved in DCM and loaded onto a silica cartridge (100 g). The cartridge was eluted with a gradient of 2M ammonia in methanol/DCM (0-15%), followed by continued elution with 2M ammonia in methanol/DCM (15%). The appropriate fractions were combined and evaporated in vacuo to give 2-[(1-methylethyl)amino]-5-[3-(2,3,4,5-tetrahydro-1H-3-benzazepin-7-yl)-1,2,4-oxadiazol-5-yl]-3-pyridinecarbonitrile (887 mg) as a white solid.

LCMS (Method formate): Retention time 0.87 min, MH⁺=375

### Preparation 121

### 1,1-Dimethylethyl 7-(5-{5-cyano-6-[(1-methylethyl)amino]-3-pyridinyl}-1,2,4-oxadiazol-3-yl)-1,2,4,5-tetrahydro-3H-3-benzazepine-3-carboxylate

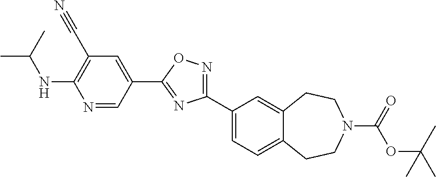

A mixture of 5-cyano-6-[(1-methylethyl)amino]-3-pyridinecarboxylic acid (Preparation 122) (1 g, 4.9 mmol), N-ethylmorpholine (1.23 ml, 9.8 mmol), HATU (2.22 g, 5.9 mmol) and 1,1-dimethylethyl 7-[(hydroxyamino)(imino)methyl]-1,2,4,5-tetrahydro-3H-3-benzazepine-3-carboxylate (Preparation 41) (1.79 g, 5.9 mmol) in DMF (10 ml) was stirred at room temperature for 4 h. The reaction mixture was diluted with ethyl acetate (50 ml) and washed with saturated sodium hydrogen carbonate, water and brine. The organic phase was dried and evaporated. The residue was dissolved in toluene (40 ml) and pyridine (10 ml) and the mixture refluxed for 2 h. The reaction mixture was cooled to room temperature and the solvent evaporated. Chromatography (ethyl acetate/hexane, 25%) gave 1,1-dimethylethyl 7-(5-{5-cyano-6-[(1-methylethyl)amino]-3-pyridinyl}-1,2,4-oxadiazol-3-yl)-1,2,4,5-tetrahydro-3H-3-benzazepine-3-carboxylate as a colourless solid (1.92 g).

LCMS (Method formate): Retention time 1.53 min, MH⁺=475 (weak)

### Preparation 122

### 5-cyano-6-[(1-methylethyl)amino]-3-pyridinecarboxylic acid

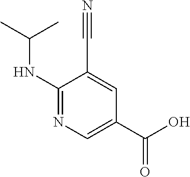

Aqueous lithium hydroxide (1M, 7.75 ml, 7.8 mmol) was added to ethyl 5-cyano-6-[(1-methylethyl)amino]-3-pyridinecarboxylate (Preparation 123) (900 mg, 3.9 mmol) in a mixture of methanol (10 ml) and water (5 ml). The reaction mixture was stirred at room temperature for 2 h 30 min and concentrated. Hydrochloric acid (5M) was added to acidify the mixture, which was extracted with a mixture of ethyl acetate and DCM (1:1, 2×100 ml). The organics were combined, dried (hydrophobic frit) and concentrated in vacuo to give 5-cyano-6-[(1-methylethyl)amino]-3-pyridinecarboxylic acid as a pale yellow solid (740 mg).

LCMS (Method formate): Retention time 0.78 min, MH⁺=206

### Preparation 123

### ethyl 5-cyano-6-[(1-methylethyl)amino]-3-pyridinecarboxylate

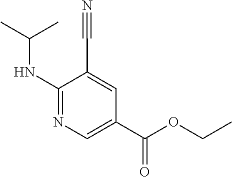

Ethyl 6-chloro-5-cyano-3-pyridinecarboxylate (820 mg, 3.9 mmol, WO2005058848) was dissolved in ethanol (10 ml), 2-propanamine (1.66 ml, 19 mmol) and triethylamine (2.72 ml, 19 mmol) were added. The mixture was heated (microwave) at 120° C. for 20 min. The reaction mixture was concentrated and partitioned between DCM (2×5 ml) and saturated sodium hydrogen carbonate solution (5 ml). The organic phases were combined, dried (hydrophobic frit) and the volatiles evaporated under a stream of nitrogen to give ethyl 5-cyano-6-[(1-methylethyl)amino]-3-pyridinecarboxylate as yellow crystals (900 mg).

LCMS (Method formate): Retention time 1.09 min, MH⁺=234

### Preparation 124

### 5-{3-[3-(2,2-dimethyl-1,3-dioxan-5-yl)-2,3,4,5-tetrahydro-1H-3-benzazepin-7-yl]-1,2,4-oxadiazol-5-yl}-2-(propyloxy)-3-pyridinecarbonitrile

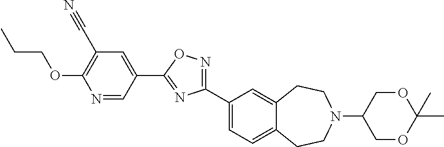

To 2-(propyloxy)-5-[3-(2,3,4,5-tetrahydro-1H-3-benzazepin-7-yl)-1,2,4-oxadiazol-5-yl]-3-pyridinecarbonitrile (Preparation 116) (150 mg, 0.40 mmol) in DCM (3 ml) was added 2,2-dimethyl-1,3-dioxan-5-one (160 mg, 1.2 mmol) and the reaction mixture stirred at room temperature for 20 min. Sodium triacetoxyborohydride (380 mg, 1.8 mmol) was added and the reaction mixture stirred at room temperature overnight. The reaction mixture was partitioned between DCM (3×10 ml) and water (10 ml). The organics were combined, dried (hydrophobic frit) and reduced to dryness under a stream of nitrogen to give 5-{3-[3-(2,2-dimethyl-1,3-dioxan-5-yl)-2,3,4,5-tetrahydro-1H-3-benzazepin-7-yl]-1,2,4-oxadiazol-5-yl}-2-(propyloxy)-3-pyridinecarbonitrile (180 mg), which was used without further purification in the subsequent reaction (Example 109).

LCMS (Method formate): Retention time 1.06 min, MH⁺=490

### Preparation 125

### 5-{3-[3-(2,2-dimethyl-1,3-dioxan-5-yl)-2,3,4,5-tetrahydro-1H-3-benzazepin-7-yl]-1,2,4-oxadiazol-5-yl}-2-(ethyloxy)-3-pyridinecarbonitrile

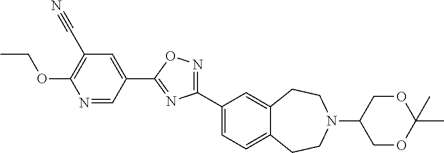

To 2-(ethyloxy)-5-[3-(2,3,4,5-tetrahydro-1H-3-benzazepin-7-yl)-1,2,4-oxadiazol-5-yl]-3-pyridinecarbonitrile (Preparation 126) (150 mg, 0.42 mmol) in DCM (3 ml) was added 2,2-dimethyl-1,3-dioxan-5-one (160 mg, 1.2 mmol) and the reaction mixture stirred at room temperature for 20 min. Sodium triacetoxyborohydride (400 mg, 1.9 mmol) was added and the reaction mixture stirred at room temperature overnight. The reaction mixture was partitioned between DCM (3×10 ml) and water (10 ml). The organic phases were combined, dried (hydrophobic frit) and reduced to dryness under a stream of nitrogen to give 5-{3-[3-(2,2-dimethyl-1,3-dioxan-5-yl)-2,3,4,5-tetrahydro-1H-3-benzazepin-7-yl]-1,2,4-oxadiazol-5-yl}-2-(ethyloxy)-3-pyridinecarbonitrile (240 mg), which was used without further purification in the subsequent reaction (Example 110).

LCMS (Method formate): Retention time 0.96 min, MH⁺=476

### Preparation 126

### 2-(ethyloxy)-5-[3-(2,3,4,5-tetrahydro-1H-3-benzazepin-7-yl)-1,2,4-oxadiazol-5-yl]-3-pyridinecarbonitrile

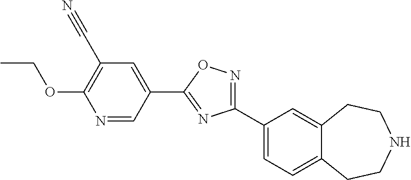

To 1,1-dimethylethyl 7-{5-[5-cyano-6-(ethyloxy)-3-pyridinyl]-1,2,4-oxadiazol-3-yl}-1,2,4,5-tetrahydro-3H-3-benzazepine-3-carboxylate (Preparation 127) (770 mg, 1.7 mmol) in DCM (15 ml) was added trifluoroacetic acid (1.5 ml, 19 mmol) and the reaction mixture stirred for 1 h at room temperature. The reaction mixture was applied to an aminopropyl SPE (50 g) and the SPE eluted with methanol in DCM (10%). The appropriate fractions were combined and the volatiles evaporated to give 2-(ethyloxy)-5-[3-(2,3,4,5-tetrahydro-1H-3-benzazepin-7-yl)-1,2,4-oxadiazol-5-yl]-3-pyridinecarbonitrile (500 mg), which was used without further purification in the subsequent reaction (Preparation 125)

LCMS (Method formate): Retention time 0.83 min, MH⁺=362

### Preparation 127

### 1,1-dimethylethyl 7-{5-[5-cyano-6-(ethyloxy)-3-pyridinyl]-1,2,4-oxadiazol-3-yl}-1,2,4,5-tetrahydro-3H-3-benzazepine-3-carboxylate

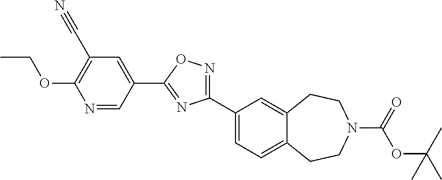

To 5-cyano-6-(ethyloxy)-3-pyridinecarboxylic acid (420 mg, 2.2 mmol, WO2005058848) and HATU (920 mg, 2.4 mmol) in DMF (10 ml) was added DIPEA (1.13 ml, 6.5 mmol) and the reaction mixture stirred at room temperature for 10 min. 1,1-Dimethylethyl 7-[(hydroxyamino)(imino)methyl]-1,2,4,5-tetrahydro-3H-3-benzazepine-3-carboxylate (Preparation 41) (790 mg, 2.6 mmol) was added and the reaction mixture stirred at room temperature for 1 h. The reaction mixture was heated at 100° C. for 1 h 30 min. The solvent was evaporated in vacuo, the residue partitioned between DCM (2×100 ml) and water (100 ml). The organic phases were combined, dried (hydrophobic frit) and evaporated in vacuo. The sample was dissolved in DCM, loaded onto a silica cartridge (100 g) and the cartridge eluted with an ethyl acetate/cyclohexane gradient (0-50%), followed by continued elution with ethyl acetate/cyclohexane (50%). The appropriate fractions were combined and evaporated in vacuo to give 1,1-dimethylethyl 7-{5-[5-cyano-6-(ethyloxy)-3-pyridinyl]-1,2,4-oxadiazol-3-yl}-1,2,4,5-tetrahydro-3H-3-benzazepine-3-carboxylate (770 mg).

LCMS (Method formate): Retention time 1.47 min, MH⁺ not seen

### Preparation 128

### 1,1-dimethylethyl {2-[7-(5-{5-cyano-6-[(1-methylethyl)amino]-3-pyridinyl}-1,2,4-oxadiazol-3-yl)-1,2,4,5-tetrahydro-3H-3-benzazepin-3-yl]-2-oxoethyl}carbamate

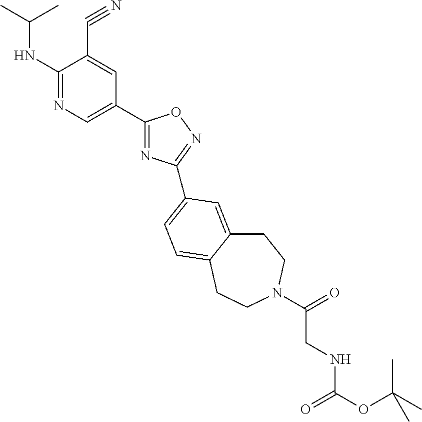

To N-BOC glycinamide (47 mg, 0.27 mmol) and HATU (100 mg, 0.27 mmol) in DMF (3 ml) was added DIPEA (0.047 ml, 0.27 mmol) and the reaction mixture stirred at room temperature for 10 min. 2-[(1-Methylethyl)amino]-5-[3-(2,3,4,5-tetrahydro-1H-3-benzazepin-7-yl)-1,2,4-oxadiazol-5-yl]-3-pyridinecarbonitrile (Preparation 120) (100 mg, 0.27 mmol) was added and the reaction mixture stirred at room temperature for 3 h. The solvent was evaporated under a stream of nitrogen. The residue was partitioned between DCM (3×10 ml) and water (10 ml). The organic phases were combined, dried (hydrophobic frit) and reduced to dryness under a stream of nitrogen to give 1,1-dimethylethyl {2-[7-(5-{5-cyano-6-[(1-methylethyl)amino]-3-pyridinyl}-1,2,4-oxadiazol-3-yl)-1,2,4,5-tetrahydro-3H-3-benzazepin-3-yl]-2-oxoethyl}carbamate (315 mg), which was used without further purification in the subsequent reaction (Example 112).

LCMS (Method formate): Retention time 1.30 min, MH⁺=532

### Preparation 129

### 1,1-dimethylethyl [2-(7-{5-[5-cyano-6-(ethyloxy)-3-pyridinyl]-1,2,4-oxadiazol-3-yl}-1,2,4,5-tetrahydro-3H-3-benzazepin-3-yl)-2-oxoethyl]carbamate

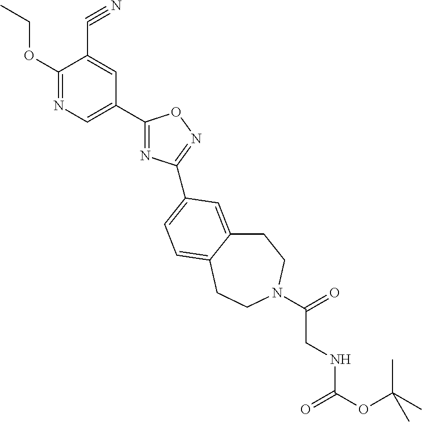

To N-BOC glycinamide (58 mg, 0.33 mmol) and HATU (160 mg, 0.42 mmol) in DMF (3 ml) was added DIPEA (0.15 ml, 0.83 mmol) and the reaction mixture stirred at room temperature for 10 min. 2-(Ethyloxy)-5-[3-(2,3,4,5-tetrahydro-1H-3-benzazepin-7-yl)-1,2,4-oxadiazol-5-yl]-3-pyridinecarbonitrile (Preparation 126) (100 mg, 0.28 mmol) was added and the reaction mixture stirred at room temperature for 3 h. The solvent was evaporated under a stream of nitrogen, the residue partitioned between DCM (3×10 ml) and water (10 ml). The organic phases were combined, dried (hydrophobic frit) and reduced to dryness under a stream of nitrogen to give 1,1-dimethylethyl [2-(7-{5-[5-cyano-6-(ethyloxy)-3-pyridinyl]-1,2,4-oxadiazol-3-yl}-1,2,4,5-tetrahydro-3H-3-benzazepin-3-yl)-2-oxoethyl]carbamate (240 mg).

LCMS (Method formate): Retention time 1.27 min, MH⁺=519

### Preparation 130

### 1,1-dimethylethyl {2-[7-(5-{3-cyano-4-[(1-methylethyl)oxy]phenyl}-1,2,4-oxadiazol-3-yl)-1,2,4,5-tetrahydro-3H-3-benzazepin-3-yl]-1,1-dimethyl-2-oxoethyl}carbamate

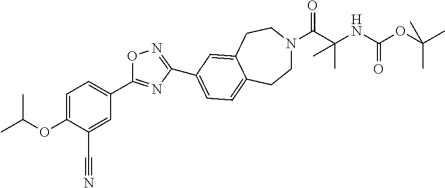

To N-{[(1,1-dimethylethyl)oxy]carbonyl}-2-methylalanine (56 mg, 0.27 mmol) and HATU (120 mg, 0.32 mmol) in DMF (1 ml) was added DIPEA (0.14 ml, 0.82 mmol) and the reaction mixture stirred at room temperature for 5 min. 2-[(1-Methylethyl)oxy]-5-[3-(2,3,4,5-tetrahydro-1H-3-benzazepin-7-yl)-1,2,4-oxadiazol-5-yl]benzonitrile hydrochloride (Preparation 43) (75 mg, 0.18 mmol) was added and the reaction mixture stirred at room temperature for 2 h. The reaction mixture was reduced to dryness under a stream of nitrogen. The sample was dissolved in DCM, applied to an aminopropyl SPE (5 g) and the SPE eluted with methanol in DCM (10%). The appropriate fractions were combined and reduced to dryness under a stream of nitrogen to give 1,1-dimethylethyl {2-[7-(5-{3-cyano-4-[(1-methylethyl)oxy]phenyl}-1,2,4-oxadiazol-3-yl)-1,2,4,5-tetrahydro-3H-3-benzazepin-3-yl]-1,1-dimethyl-2-oxoethyl}carbamate (130 mg) as a brown gum, which was used without further purification in the subsequent reaction (Example 117).

LCMS (Method formate): Retention time 1.33 min, MH⁺=560

### Preparation 131

### 5-{3-[2-(brornoacetyl)-5-methyl-1,2,3,4-tetrahydro-6-isoquinolinyl]-1,2,4-oxadiazol-5-yl}-2-[(1-methylethyl)oxy]benzonitrile

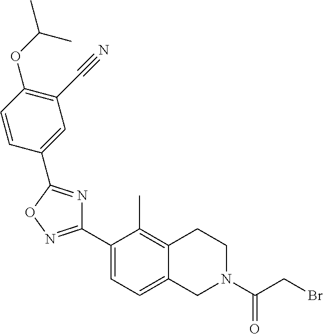

A solution of bromoacetyl bromide (360 μl, 4.1 mmol) in DCM (2 ml) was added dropwise to a solution of 2-[(1-methylethyl)oxy]-5-[3-(5-methyl-1,2,3,4-tetrahydro-6-isoquinolinyl)-1,2,4-oxadiazol-5-yl]benzonitrile trifluoroacetic acid salt (Preparation 25) (2.0 g, 4.1 mmol) and DIPEA (1.79 ml, 10 mmol) in DCM (20 ml). The reaction mixture was stirred at room temperature for 1 h. The solvent was evaporated and the residue dissolved in ethyl acetate (25 ml). The solution was washed with water and brine. The organic phase was dried and evaporated. The residue was chromatographed (methanol/DCM, 0-5%) the residual oil, dissolved in acetonitrile (10 ml) and left overnight. The solid was isolated by filtration and dried to give 5-{3-[2-(bromoacetyl)-5-methyl-1,2,3,4-tetrahydro-6-isoquinolinyl]-1,2,4-oxadiazol-5-yl}-2-[(1-methylethyl)oxy]benzonitrile as a light brown solid (900 mg).

LCMS (Method formate): Retention time 1.28 min, MH⁺=495/497

### Preparation 132

### 2-(bromoacetyl)-6-(5-{3-chloro-4-[(1-methylethyl)oxy]phenyl}-1,2,4-oxadiazol-3-yl)-5-methyl-1,2,3,4-tetrahydroisoquinoline

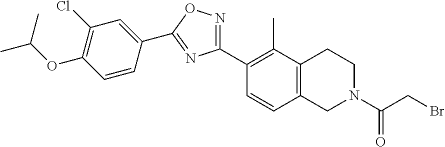

A solution of 6-(5-{3-chloro-4-[(1-methylethyl)oxy]phenyl}-1,2,4-oxadiazol-3-yl)-5-methyl-1,2,3,4-tetrahydroisoquinoline hydrochloride (Preparation 12) (720 mg, 1.7 mmol) and DIPEA (0.89 ml, 5.1 mmol) in DCM (10 ml) was treated with a dropwise addition of bromoacetyl bromide (0.16 ml, 1.9 mmol) in DCM (5 ml). After 10 min, most of the solvent was evaporated in vacuo and the residue dissolved with ethyl acetate. The organic phase was washed with hydrochloric acid (2N). The aqueous was exacted (x2), the combined organic washed with sodium hydrogen carbonate, brine, dried (MgSO₄) and concentrated in vacuo to a brown foam. An attempt to dry the material in vacuo resulted in some loss of material into the vacuum line. The remaining material (600 mg) was purified by chromatography on a column (10 g), eluting with an ethyl acetate/cyclohexane gradient to give 2-(bromoacetyl)-6-(5-{3-chloro-4-[(1-methylethyl)oxy]phenyl}-1,2,4-oxadiazol-3-yl)-5-methyl-1,2,3,4-tetrahydroisoquinoline (300 mg) as a pale yellow oil.

LCMS (Method HpH): Retention time 1.47 min, MH⁺=460/462

The material recovered from the vacuum line was purified by chromatography on a column (25 g), eluting with an ethyl acetate/cyclohexane gradient to give 2-(bromoacetyl)-6-(5-{3-chloro-4-[(1-methylethyl)oxy]phenyl}-1,2,4-oxadiazol-3-yl)-5-methyl-1,2,3,4-tetrahydroisoquinoline (94 mg) as a pale yellow oil.

LCMS (Method HpH): Retention time 1.47 min, MH⁺=460/462

### Preparation 133

### 1,1-dimethylethyl {2-[7-(5-{3-cyano-4-[(1-methylethyl)oxy]phenyl}-1,2,4-oxadiazol-3-yl)-6-methyl-1,2,4,5-tetrahydro-3H-3-benzazepin-3-yl]-2-oxoethyl}carbamate

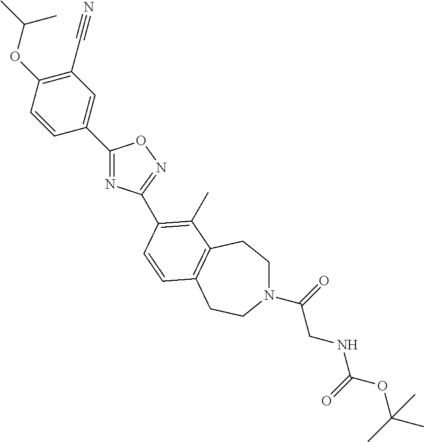

A mixture of N-bocglycine (44 mg, 0.25 mmol), N-ethylmorpholine (64 μl, 0.5 mmol), hydroxybenzotriazole hydrate (46 mg, 0.3 mmol), EDC (58 mg, 0.3 mmol) and 2-[(1-methylethyl)oxy]-5-[3-(6-methyl-2,3,4,5-tetrahydro-1H-3-benzazepin-7-yl)-1,2,4-oxadiazol-5-yl]benzonitrile trifluoroacetate (Preparation 134) (150 mg, 0.3 mmol) in DMF (3 ml) was stirred at room temperature over the weekend. The reaction mixture was diluted with saturated sodium hydrogen carbonate (5 ml) and extracted with ethyl acetate (3×5 ml). The combined organic extracts were washed with hydrochloric acid (1M), water and brine. The organic phase was dried and evaporated. Chromatography (methanol/DCM, 0-4%) gave 1,1-dimethylethyl {2-[7-(5-{3-cyano-4-[(1-methylethyl)oxy]phenyl}-1,2,4-oxadiazol-3-yl)-6-methyl-1,2,4,5-tetrahydro-3H-3-benzazepin-3-yl]-2-oxoethyl}carbamate as a colourless oil (110 mg).

LCMS (Method formate): Retention time 1.34 min, MH⁺=546

### Preparation 134

### 2-[(1-methylethyl)oxy]-5-[3-(6-methyl-2,3,4,5-tetrahydro-1H-3-benzazepin-7-yl)-1,2,4-oxadiazol-5-yl]benzonitrile trifluoroacetate

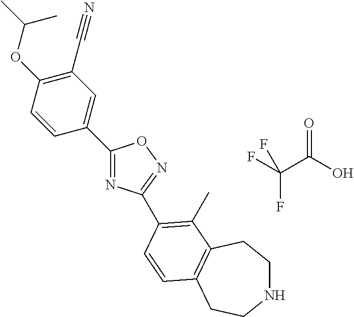

Trifluoroacetic acid (5 ml) was added slowly to a stirred solution of 1,1-dimethylethyl 7-(5-{3-cyano-4-[(1-methylethyl)oxy]phenyl}-1,2,4-oxadiazol-3-yl)-6-methyl-1,2,4,5-tetrahydro-3H-3-benzazepine-3-carboxylate (Preparation 135) (2.7 g, 5.5 mmol) in DCM (10 ml). The reaction mixture was stirred at room temperature for 2 h. The solvent was evaporated and the residue re-evaporated from toluene (x2). Trituration with diethyl ether gave 2-[(1-methylethyl)oxy]-5-[3-(6-methyl-2,3,4,5-tetrahydro-1H-3-benzazepin-7-yl)-1,2,4-oxadiazol-5-yl]benzonitrile trifluoroacetate as a colourless solid (2.4 g).

LCMS (Method formate): Retention time 0.92 min, MH⁺=389

### Preparation 135

### 1,1-dimethylethyl 7-(5-{3-cyano-4-[(1-methylethyl)oxy]phenyl}-1,2,4-oxadiazol-3-yl)-6-methyl-1,2,4,5-tetrahydro-3H-3-benzazepine-3-carboxylate

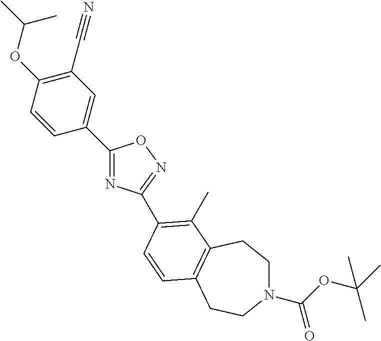

3-cyano-4-[(1-methylethyl)oxy]benzoyl chloride (Preparation 169) (1.84 g, 8.2 mmol, WO2009080730/WO2008128951) was added portionwise to a stirred solution of 1,1-dimethylethyl 7-[(hydroxyamino)(imino)methyl]-6-methyl-1,2,4,5-tetrahydro-3H-3-benzazepine-3-carboxylate (Preparation 136) (2.5 g, 7.8 mmol) in toluene (20 ml) and pyridine (10 ml). The reaction mixture was refluxed for 3 h. The reaction mixture was cooled and the solvent evaporated. The residue was chromatographed (ethyl acetate/iso-hexane, 5-25%) to give 1,1-dimethylethyl 7-(5-{3-cyano-4-[(1-methylethyl)oxy]phenyl}-1,2,4-oxadiazol-3-yl)-6-methyl-1,2,4,5-tetrahydro-3H-3-benzazepine-3-carboxylate as a colourless solid (2.7 g).

LCMS (Method formate): Retention time 1.49 min, MH⁺=489 (weak)

### Preparation 136

### 1,1-dimethylethyl 7-[(hydroxyamino)(imino)methyl]-6-methyl-1,2,4,5-tetrahydro-3H-3-benzazepine-3-carboxylate

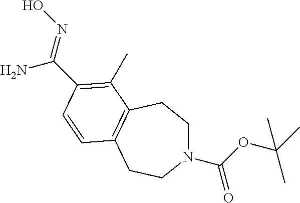

A mixture of 1,1-dimethylethyl 7-cyano-6-methyl-1,2,4,5-tetrahydro-3H-3-benzazepine-3-carboxylate (Preparation 137) (3.0 g, 10 mmol), hydroxylamine hydrochloride (2.18 g, 31 mmol) and sodium hydrogen carbonate (2.64 g, 31 mmol) in ethanol (50 ml) was refluxed for 24 h. Hydroxylamine hydrochloride (1 g) and sodium hydrogen carbonate (1 g) were added and reflux continued for 24 h. The reaction mixture was cooled to room temperature and filtered. The solvent was evaporated from the filtrate. Water (50 ml) was added to the residue and the mixture extracted with ethyl acetate (2×50 ml). The combined organic extracts were washed with water and brine, dried and evaporated to give 1,1-dimethylethyl 7-[(hydroxyamino)(imino)methyl]-6-methyl-1,2,4,5-tetrahydro-3H-3-benzazepine-3-carboxylate as a colourless foam (3.3 g).

LCMS (Method HpH): Retention time 0.94 min, MH⁺=320

### Preparation 137

### 1,1-dimethylethyl 7-cyano-6-methyl-1,2,4,5-tetrahydro-3H-3-benzazepine-3-carboxylate

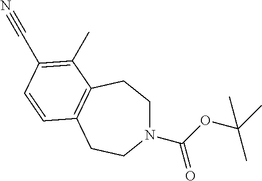

A solution of 1,1-dimethylethyl 6-methyl-7-{[(trifluoromethyl)sulfonyl]oxy}-1,2,4,5-tetrahydro-3H-3-benzazepine-3-carboxylate (Preparation 138) (13 g, 32 mmol) in DMF (150 ml) was degassed by alternately evacuating and purging with nitrogen (x3), then zinc cyanide (4.85 g, 41 mmol) and tetrakis(triphenylphosphine) palladium (3.67 g, 3.2 mmol) were added and the mixture was heated at 120° C. for 6 h. The mixture was cooled, diluted with ethyl acetate (400 ml) and filtered. The filtrate was washed with water (2×200 ml), dried and evaporated to an orange semi-solid, which was purified by chromatography on a silica cartridge (330 g) eluting with an ethyl acetate/cyclohexane gradient (0-50% ethyl acetate) to give 1,1-dimethylethyl 7-cyano-6-methyl-1,2,4,5-tetrahydro-3H-3-benzazepine-3-carboxylate as a white solid (8.6 g, 95%).

LCMS (Method formate): Retention time 1.21 min, MH⁺=287

### Preparation 138

### 1,1-dimethylethyl 6-methyl-7-{[(trifluoromethyl)sulfonyl]oxy}-1,2,4,5-tetrahydro-3H-3-benzazepine-3-carboxylate

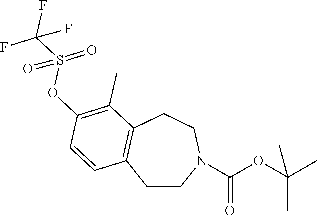

Triflic anhydride (9.5 ml, 56 mmol) was added dropwise to a solution of 1,1-dimethylethyl 7-hydroxy-6-methyl-1,2,4,5-tetrahydro-3H-3-benzazepine-3-carboxylate (12 g, 43 mmol, WO2005118549) and pyridine (7 ml, 87 mmol) in DCM (200 ml) at −50° C., then the mixture was allowed to warm up slowly to room temperature, giving an amber solution. This was washed with water and hydrochloric acid (1M), dried and evaporated to a brown solid, which was purified by chromatography on a silica cartridge (330 g) eluting with an ethyl acetate/cyclohexane gradient (0-50%) to give 1,1-dimethylethyl 6-methyl-7-{[(trifluoromethyl)sulfonyl]oxy}-1,2,4,5-tetrahydro-3H-3-benzazepine-3-carboxylate (15.0 g, 85%) as colourless solid.

LCMS (Method formate): Retention time 1.46 min, MH⁺=410

### Preparation 139

### 1,1-dimethylethyl {2-[6-(5-{5-chloro-6-[(1-methylethyl)oxy]-3-pyridinyl}-1,2,4-oxadiazol-3-yl)-5-methyl-3,4-dihydro-2(1H)-isoquinolinyl]-2-oxoethyl}carbamate

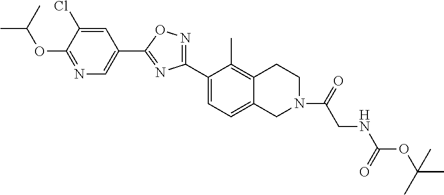

To 6-(5-{5-chloro-6-[(1-methylethyl)oxy]-3-pyridinyl}-1,2,4-oxadiazol-3-yl)-5-methyl-1,2,3,4-tetrahydroisoquinoline (Preparation 140) (50 mg, 0.13 mmol) and HATU (74 mg, 0.20 mmol) in DMF (2 ml) was added DIPEA (0.068 ml, 0.39 mmol) and the reaction mixture stirred at room temperature for 10 min. N-BOC glycinamide (27 mg, 0.16 mmol) was added and the reaction mixture stirred at room temperature for 3 h. The solvent was evaporated under a stream of nitrogen. The residue was partitioned between DCM (3×10 ml) and water (10 ml). The organics were combined, dried (hydrophobic frit) and the solvent evaporated under a stream of nitrogen to give 1,1-dimethylethyl {2-[6-(5-{5-chloro-6-[(1-methylethyl)oxy]-3-pyridinyl}-1,2,4-oxadiazol-3-yl)-5-methyl-3,4-dihydro-2(1H)-isoquinolinyl]-2-oxoethyl}carbamate (62 mg, 88%) which was used in the subsequent reaction (Example 141) without further purification.

LCMS (Method formate): Retention time 1.48 min, MH⁺=542/544

### Preparation 140

### 6-(5-{5-chloro-6-[(1-methylethyl)oxy]-3-pyridinyl}-1,2,4-oxadiazol-3-yl)-5-methyl-1,2,3,4-tetrahydroisoquinoline

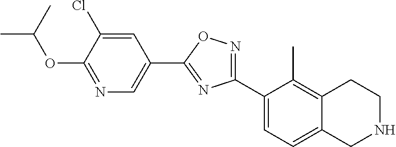

To 1,1-dimethylethyl 6-(5-{5-chloro-6-[(1-methylethyl)oxy]-3-pyridinyl}-1,2,4-oxadiazol-3-yl)-5-methyl-3,4-dihydro-2(1H)-isoquinolinecarboxylate (Preparation 141) (2.02 g, 4.2 mmol) in DCM (18 ml) was added trifluoroacetic acid (2 ml, 26 mmol) and the reaction mixture was stirred at room temperature for 1.5 h. Further trifluoroacetic acid (2 ml, 26 mmol) was added and the reaction mixture stirred at room temperature for 30 min. The reaction mixture was applied to a silica cartridge (50 g) and eluted using methanol in DCM (10%, then 50%). The appropriate fractions were combined and evaporated in vacuo. The residue was dissolved in DCM, applied to an aminopropyl SPE (50 g) and the SPE eluted using methanol in DCM (10%). The appropriate fractions were combined and reduced to dryness under a stream of nitrogen to give 6-(5-{5-chloro-6-[(1-methylethyl)oxy]-3-pyridinyl}-1,2,4-oxadiazol-3-yl)-5-methyl-1,2,3,4-tetrahydroisoquinoline (1.4 g, 87%) as a cream solid.

LCMS (Method formate): Retention time 1.08 min, MH⁺=385/387

### Preparation 141

### 1,1-dimethylethyl 6-(5-{5-chloro-6-[(1-methylethyl)oxy]-3-pyridinyl}-1,2,4-oxadiazol-3-yl)-5-methyl-3,4-dihydro-2(1H)-isoquinolinecarboxylate

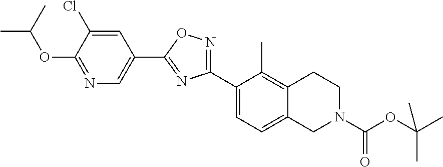

5-Chloro-6-[(1-methylethyl)oxy]-3-pyridinecarboxylic acid (1.3 g, 6.0 mmol, Enamine) and HATU (2.57 g, 6.8 mmol) were combined in DMF (10 ml), DIPEA (3.16 ml, 18 mmol) was added, and the reaction mixture stirred at room temperature for 10 min. 1,1-Dimethylethyl 6-[(hydroxyamino)(imino)methyl]-5-methyl-3,4-dihydro-2(1H)-isoquinolinecarboxylate (Preparation 23) (2.21 g, 7.2 mmol, WO2009080724) was added and the reaction mixture stirred at room temperature for 1.5 h then heated at 100° C. overnight. The solvent was evaporated under vacuum, and the residue partitioned between DCM (3×100 ml) and saturated aqueous sodium hydrogen carbonate solution (100 ml). The organics were combined, dried (hydrophobic frit) and evaporated in vacuo. The residue was dissolved in DCM and applied to a silica cartridge (100 g), the cartridge was eluted with an ethyl acetate/cyclohexane gradient (0-25%) followed by continued elution with ethyl acetate/cyclohexane (25%). The appropriate fractions were combined and evaporated in vacuo to give 1,1-dimethylethyl 6-(5-{5-chloro-6-[(1-methylethyl)oxy]-3-pyridinyl}-1,2,4-oxadiazol-3-yl)-5-methyl-3,4-dihydro-2(1H)-isoquinolinecarboxylate (2.02 g, 69%) as a cream solid.

LCMS (Method formate): Retention time 1.68 min, MH⁺ not seen

### Preparation 142

### 2-(propylamino)-5-[3-(2,3,4,5-tetrahydro-1H-3-benzazepin-7-yl)-1,2,4-oxadiazol-5-yl]-3-pyridinecarbonitrile

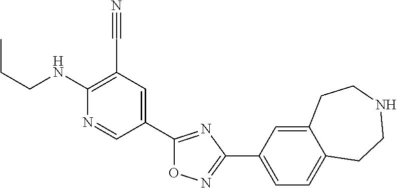

To 1,1-dimethylethyl 7-{5-[5-cyano-6-(propylamino)-3-pyridinyl]-1,2,4-oxadiazol-3-yl}-1,2,4,5-tetrahydro-3H-3-benzazepine-3-carboxylate (Preparation 143) (440 mg, 0.93 mmol) in DCM (9 ml) was added trifluoroacetic acid (1 ml, 13 mmol) and the reaction mixture was stirred at room temperature for 40 min. The solvent was evaporated from the mixture in vacuo and the residue was dissolved in DCM and applied to an aminopropyl cartridge (10 g) and which was then eluted with methanol in DCM (10%). The appropriate fractions were combined and evaporated in vacuo to give 2-(propylamino)-5-[3-(2,3,4,5-tetrahydro-1H-3-benzazepin-7-yl)-1,2,4-oxadiazol-5-yl]-3-pyridinecarbonitrile as a cream solid, (320 mg, 92%).

LCMS (Method formate): Retention time 0.89 min, MH⁺=375

### Preparation 143

### 1,1-dimethylethyl 7-{5-[5-cyano-6-(propylamino)-3-pyridinyl]-1,2,4-oxadiazol-3-yl}-1,2,4,5-tetrahydro-3H-3-benzazepine-3-carboxylate

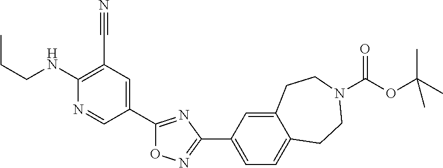

To a solution of 5-cyano-6-(propylamino)-3-pyridinecarboxylic acid (Preparation 144) (400 mg, 1.9 mmol) in DMF (10 ml) was added DIPEA (1.20 ml, 6.8 mmol) and 1,1-dimethylethyl 7-[(hydroxyamino)(imino)methyl]-1,2,4,5-tetrahydro-3H-3-benzazepine-3-carboxylate (Preparation 41) (710 mg, 2.3 mmol). HATU (1.10 g, 2.9 mmol) was added to the mixture which was stirred at room temperature under nitrogen for 45 min then heated at 100° C. for 1.5 h. After allowing to cool to room temperature and standing for 20 h the volatiles were evaporated in vacuo and the residue partitioned between DCM (30 ml) and aqueous saturated sodium hydrogen carbonate solution (30 ml). The aqueous phase was further extracted with DCM (2×30 ml), the combined organic phases dried (hydrophobic frit), and the solvent evaporated in vacuo to give a residue which was purified by flash chromatography, eluting with an ethyl acetate/cyclohexane gradient (0-25% ethyl acetate). The required fractions were combined and the solvent evaporated in vacuo to give 1,1-dimethylethyl 7-{5-[5-cyano-6-(propylamino)-3-pyridinyl]-1,2,4-oxadiazol-3-yl}-1,2,4,5-tetrahydro-3H-3-benzazepine-3-carboxylate as a pale yellow solid (450 mg, 49%).

LCMS (Method formate): Retention time 1.55 min, MH⁺=475

### Preparation 144

### 5-cyano-6-(propylamino)-3-pyridinecarboxylic acid

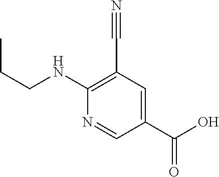

Aqueous lithium hydroxide (1M, 9.40 ml, 9.4 mmol) solution was added to methyl 5-cyano-6-(propylamino)-3-pyridinecarboxylate (Preparation 145) (1.03 g, 4.7 mmol) in methanol (10 ml). The reaction mixture was stirred at room temperature for 2.25 h. Further aqueous lithium hydroxide (1 M, 9.4 ml, 9.4 mmol) was added and the mixture stirred for 3.25 h. The mixture was stirred at room temperature for a further 0.5 h before being placed into a freezer (−22° C.) for 64 h. After removal from the freezer and stirring for 5.5 h at room temperature, the mixture was concentrated in vacuo to remove the methanol, before acidification with hydrochloric acid (5M). The mixture was extracted with ethyl acetate (3×100 ml), the combined organic phases dried (hydrophobic frit) and the solvent evaporated in vacuo. The solid residue was purified by flash chromatography on a silica cartridge (40 g), eluting with a gradient of 1% acetic acid in ethyl acetate/cyclohexane (0-100%). The required fractions were combined and evaporated in vacuo to give material which was dissolved in a minimum of methanol and ethyl acetate (4:1). Florisil was added until a slurry was obtained and the solvent was evaporated in vacuo to give a powder which was applied a silica cartridge (50 g). The cartridge was eluted with an ethyl acetate/cyclohexane gradient (0-100%) followed by a gradient of methanol/DCM (0-50%). The required fractions were combined and the solvent evaporated in vacuo to give 5-cyano-6-(propylamino)-3-pyridinecarboxylic acid as a pale yellow solid (400 mg, 42%).

LCMS (Method formate): Retention time 0.79 min, MH⁺=206

### Preparation 145

### methyl 5-cyano-6-(propylamino)-3-pyridinecarboxylate

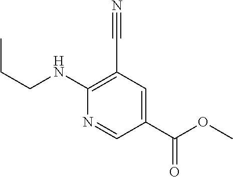

Methyl 6-chloro-5-cyano-3-pyridinecarboxylate (Preparation 146) (0.94 g, 4.8 mmol) was dissovled in methanol (8 ml) and 1-propylamine (1.78 ml, 24 mmol) and triethylamine (3.36 ml, 24 mmol) were added. The mixture was heated in a (microwave) with stirring at 120° C. for 20 min. The solvent was evaporated in vacuo and the residue was partitioned between saturated sodium hydrogen carbonate solution (10 ml) and DCM (15 ml). The phases were separated and the aqueous phase further extracted with DCM (2×15 ml). The combined organic phases were dried (hydrophobic frit) and the solvent evaporated in vacuo to give methyl 5-cyano-6-(propylamino)-3-pyridinecarboxylate as a light brown solid, (1.03 g, 98%).

LCMS (Method formate): Retention time 1.01 min, MH⁺=220

### Preparation 146

### Methyl 6-chloro-5-cyano-3-pyridinecarboxylate

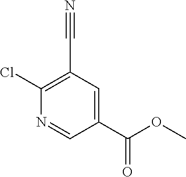

A mixture of methyl 5-cyano-6-oxo-1,6-dihydro-3-pyridinecarboxylate (1.22 g, 6.8 mmol, Enamine) and phosphorus oxychloride (10 ml, 110 mmol) was heated at 100° C. for 3 h under nitrogen. After allowing to cool to room temperature, the reaction mixture was added slowly, with vigorous stirring, to a solution of sodium acetate (55 g, 680 mmol) in water (200 ml). The solution was cooled (ice bath) and after 15 min was extracted with DCM (4×100 ml). The combined organic phases were washed with water (100 ml), dried (sodium sulphate overnight, then hydrophobic frit) and evaporated in vacuo to give methyl 6-chloro-5-cyano-3-pyridinecarboxylate as a light brown solid (950 mg).

LCMS (Method formate): Retention time 0.85 min, MH⁺ not seen

### Preparation 147

### 6-(5-{5-chloro-6-[(1-methylethyl)oxy]-3-pyridinyl}-1,2,4-oxadiazol-3-yl)-2-(2,2-dimethyl-1,3-dioxan-5-yl)-5-methyl-1,2,3,4-tetrahydroisoquinoline

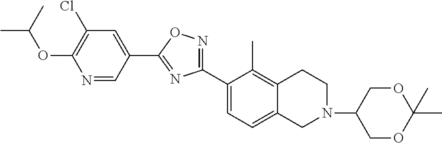

To 6-(5-{5-chloro-6-[(1-methylethyl)oxy]-3-pyridinyl}-1,2,4-oxadiazol-3-yl)-5-methyl-1,2,3,4-tetrahydroisoquinoline (Preparation 140) (100+mg, 0.26 mmol) in DCM (3 ml) was added 2,2-dimethyl-1,3-dioxan-5-one (100 mg, 0.78 mmol) and the reaction mixture stirred at room temperature for 20 min. Sodium triacetoxyborohydride (250 mg, 1.2 mmol) was added and the reaction mixture stirred at room temperature overnight. The reaction mixture was partitioned between DCM (3×10 ml) and water (10 ml). The organics were combined, dried (hydrophobic frit) and reduced to dryness under a stream of nitrogen to give 6-(5-{5-chloro-6-[(1-methylethyl)oxy]-3-pyridinyl}-1,2,4-oxadiazol-3-yl)-2-(2,2-dimethyl-1,3-dioxan-5-yl)-5-methyl-1,2,3,4-tetrahydroisoquinoline (190 mg, 147%), which was used without further purification in the subsequent reaction (Example 144).

LCMS (Method formate): Retention time 1.08 min, MH⁺=499/501

### Preparation 148

### 5-[3-(6-methyl-2,3,4,5-tetrahydro-1H-3-benzazepin-7-yl)-1,2,4-oxadiazol-5-yl]-2-(propylamino)-3-pyridinecarbonitrile trifluoroacetate

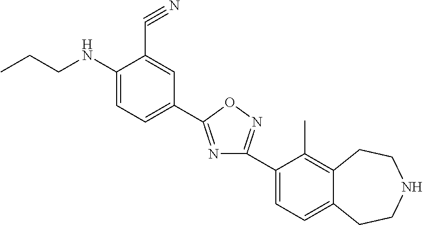

Trifluoroacetic acid (1 ml, 13 mmol) was added to 1,1-dimethylethyl 7-{5-[5-cyano-6-(propylamino)-3-pyridinyl]-1,2,4-oxadiazol-3-yl}-6-methyl-1,2,4,5-tetrahydro-3H-3-benzazepine-3-carboxylate (Preparation 149) (210 mg, 0.42 mmol) in DCM (5 ml) and the mixture was stirred at room temperature for 2 h. The reaction mixture was applied to an aminopropyl SPE (5 g) and the SPE eluted with methanol/DCM (10%). The appropriate fractions were combined and evaporated in vacuo, and further dried under vacuum to give 5-[3-(6-methyl-2,3,4,5-tetrahydro-1H-3-benzazepin-7-yl)-1,2,4-oxadiazol-5-yl]-2-(propylamino)-3-pyridinecarbonitrile trifluoroacetate as an off-white solid (230 mg), which was used in the subsequent reaction without further purification.

LCMS (Method formate): Retention time 0.90 min, MH⁺=389

### Preparation 149

### 1,1-dimethylethyl 7-{5-[5-cyano-6-(propylamino)-3-pyridinyl]-1,2,4-oxadiazol-3-yl}-6-methyl-1,2,4,5-tetrahydro-3H-3-benzazepine-3-carboxylate

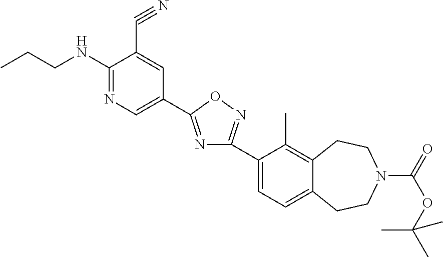

To 5-cyano-6-(propylamino)-3-pyridinecarboxylic acid (Preparation 144) (0.54 g, 2.6 mmol) and HATU (1.11 g, 2.9 mmol) in DMF (9 ml) was added DIPEA (1.37 ml, 7.8 mmol). The reaction was stirred for 15 min at room temperature. 1,1-Dimethylethyl 7-[(hydroxyamino)(imino)methyl]-6-methyl-1,2,4,5-tetrahydro-3H-3-benzazepine-3-carboxylate (Preparation 136) (0.88 g, 2.8 mmol) was added and the mixture was stirred for 3 h. The reaction was stirred and heated at 100° C. for 1.5 h and the mixture was left to stand overnight. The solvent was evaporated in vacuo and the mixture was partitioned between DCM (3×60 ml) and water (60 ml). The organics were combined, dried (hydrophobic frit) and the solvent evaporated in vacuo. The residue was dissolved in methanol/DCM (10%) and applied to a silica cartridge (100 g). The cartridge was eluted with an ethyl acetate/cyclohexane gradient (0-50%), followed by continued elution with ethyl acetate/cyclohexane (50%). The appropriate fractions were combined and evaporated in vacuo and the residue dried under vacuum to give 1,1-dimethylethyl 7-{5-[5-cyano-6-(propylamino)-3-pyridinyl]-1,2,4-oxadiazol-3-yl}-6-methyl-1,2,4,5-tetrahydro-3H-3-benzazepine-3-carboxylate (210 mg) as an off-white solid.

LCMS (Method formate): Retention time 1.53 min, MH⁺=489

### Preparation 150

### [7-(5-{3-cyano-4-[(1-methylethyl)oxy]phenyl}-1,2,4-oxadiazol-3-yl)-6-methyl-1,2,4,5-tetrahydro-3H-3-benzazepin-3-yl]acetic acid trifluoroacetate

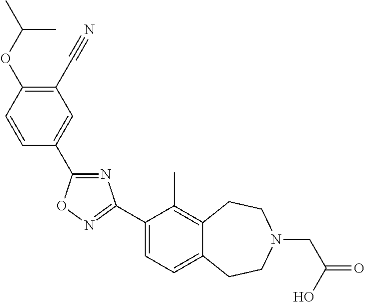

A solution of 1,1-dimethylethyl [7-(5-{3-cyano-4-[(1-methylethyl)oxy]phenyl}-1,2,4-oxadiazol-3-yl)-6-methyl-1,2,4,5-tetrahydro-3H-3-benzazepin-3-yl]acetate (Preparation 151) (400 mg, 0.8 mmol) in DCM (8 ml) and trifluoroacetic acid (2 ml) was stirred at room temperature overnight. The solvent was evaporated and the residue re-evaporated from toluene (x2). Trituration of the residue with diethyl ether gave [7-(5-{3-cyano-4-[(1-methylethyl)oxy]phenyl}-1,2,4-oxadiazol-3-yl)-6-methyl-1,2,4,5-tetrahydro-3H-3-benzazepin-3-yl]acetic acid trifluoroacetate as a colourless solid (400 mg).

LCMS (Method formate): Retention time 0.94 min, MH⁺=447

### Preparation 151

### 1,1-dimethylethyl [7-(5-{3-cyano-4-[(1-methylethyl)oxy]phenyl}-1,2,4-oxadiazol-3-yl)-6-methyl-1,2,4,5-tetrahydro-3H-3-benzazepin-3-yl]acetate

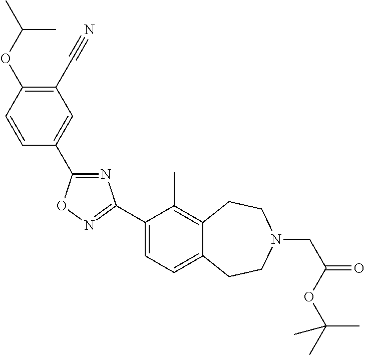

A mixture of 2-[(1-methylethyl)oxy]-5-[3-(6-methyl-2,3,4,5-tetrahydro-1H-3-benzazepin-7-yl)-1,2,4-oxadiazol-5-yl]benzonitrile trifluoroacetate (Preparation 134) (800 mg, 1.6 mmol), t-butyl bromoacetate (235 μl, 1.6 mmol) and potassium carbonate (550 mg, 4.0 mmol) was stirred at room temperature overnight. The reaction mixture was partitioned between ethyl acetate (10 ml) and water (10 ml). The organic phase was separated, dried and evaporated. The residue was chromatographed (ethyl acetate/isohexane, 5-20%) to give 1,1-dimethylethyl [7-(5-{3-cyano-4-[(1-methylethyl)oxy]phenyl}-1,2,4-oxadiazol-3-yl)-6-methyl-1,2,4,5-tetrahydro-3H-3-benzazepin-3-yl]acetate as a colourless solid (410 mg).

LCMS (Method formate): Retention time 1.02 min, MH⁺=503

### Preparation 152

### 5-{3-[3-(2,2-dimethyl-1,3-dioxan-5-yl)-6-methyl-2,3,4,5-tetrahydro-1H-3-benzazepin-7-yl]-1,2,4-oxadiazol-5-yl}-2-(propylamino)-3-pyridinecarbonitrile

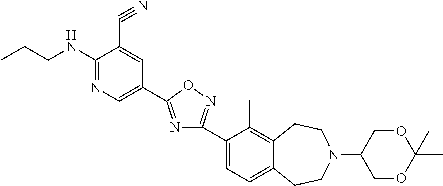

2,2-Dimethyl-1,3-dioxan-5-one (0.12 ml, 0.99 mmol) was added to 5-[3-(6-methyl-2,3,4,5-tetrahydro-1H-3-benzazepin-7-yl)-1,2,4-oxadiazol-5-yl]-2-(propylamino)-3-pyridinecarbonitrile trifluoroacetate (Preparation 148) (160 mg, 0.40 mmol) in DCM (4 ml) and the reaction was left to stir at room temperature for 1 h. Sodium triacetoxyborohydride (310 mg, 1.5 mmol) was added and the reaction was stirred for 2 h and then at room temperature over the weekend. The mixture was partitioned between DCM (3×15 ml) and water (15 ml). The organic phases were combined, dried (hydrophobic frit) and evaporated in vacuo to give a yellow gum. The sample was dissolved in DCM and applied to a silica cartridge (25 g). The cartridge was eluted with a methanol/DCM (0-10%) gradient, followed bycontinued elution with methanol/DCM (10%). The appropriate fractions were combined and evaporated in vacuo to give 5-{3-[3-(2,2-dimethyl-1,3-dioxan-5-yl)-6-methyl-2,3,4,5-tetrahydro-1H-3-benzazepin-7-yl]-1,2,4-oxadiazol-5-yl}-2-(propylamino)-3-pyridinecarbonitrile (161 mg) as a yellow oil which solidified. This was used without further purification in the subsequent reaction (Example 149).

LCMS (Method formate): Retention time 1.03 min, MH⁺=503

### Preparation 153

### 5-{3-[3-(2,2-dimethyl-1,3-dioxan-5-yl)-2,3,4,5-tetrahydro-1H-3-benzazepin-7-yl]-1,2,4-oxadiazol-5-yl}-2-(propylamino)-3-pyridinecarbonitrile

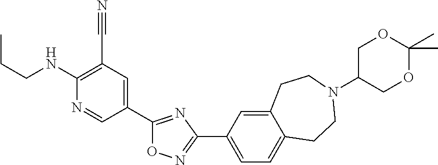

To a stirred suspension of 2-(propylamino)-5-[3-(2,3,4,5-tetrahydro-1H-3-benzazepin-7-yl)-1,2,4-oxadiazol-5-yl]-3-pyridinecarbonitrile (Preparation 142) (140 mg, 0.36 mmol) in DCM (3 ml) under nitrogen was added 2,2-dimethyl-1,3-dioxan-5-one (0.13 ml, 1.1 mmol) followed by sodium triacetoxyborohydride (340 mg, 1.6 mmol) and acetic acid (0.031 ml, 0.54 mmol). The mixture was stirred at room temperature under nitrogen for 65 h. Saturated aqueous sodium hydrogen carbonate solution (5 ml) was added to the mixture which was stirred vigorously for 15 min. The phases were separated and the aqueous phase was extracted with DCM (3×4 ml). The combined organic phases were dried (hydrophobic frit), and the solvent was evaporated under a stream of nitrogen to give 5-{3-[3-(2,2-dimethyl-1,3-dioxan-5-yl)-2,3,4,5-tetrahydro-1H-3-benzazepin-7-yl]-1,2,4-oxadiazol-5-yl}-2-(propylamino)-3-pyridinecarbonitrile as a cream solid (180 mg). This material was used in the subsequent reaction (Example 150) without further purification.

LCMS (Method formate): Retention time 1.00 min, MH⁺=489

### Preparation 154

### 1,1-dimethylethyl [2-(7-{5-[5-cyano-6-(propylamino)-3-pyridinyl]-1,2,4-oxadiazol-3-yl}-1,2,4,5-tetrahydro-3H-3-benzazepin-3-yl)-2-oxoethyl]carbamate

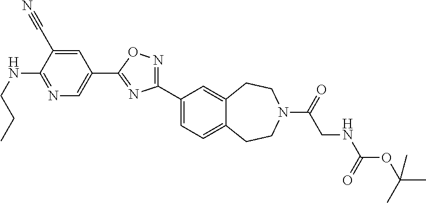

To N-BOC glycine (30 mg, 0.17 mmol) and HATU (79 mg, 0.21 mmol) in DMF (2 ml) was added DIPEA (0.070 ml, 0.40 mmol) and the reaction mixture stirred at room temperature for 10 min. 2-(Propylamino)-5-[3-(2,3,4,5-tetrahydro-1H-3-benzazepin-7-yl)-1,2,4-oxadiazol-5-yl]-3-pyridinecarbonitrile (Preparation 142) (50 mg, 0.13 mmol) was added to the mixture which was then stirred at room temperature for a further 15 min. The solvent was evaporated under a stream of nitrogen and the residue was partitioned between DCM (5 ml) and saturated aqueous sodium hydrogen carbonate solution (5 ml). The phases were separated and the aqueous phase further extracted with DCM (2×5 ml). The combined organic phases were dried (hydrophobic frit) and the solvent was evaporated to dryness under a stream of nitrogen to give 1,1-dimethylethyl [2-(7-{5-[5-cyano-6-(propylamino)-3-pyridinyl]-1,2,4-oxadiazol-3-yl}-1,2,4,5-tetrahydro-3H-3-benzazepin-3-yl)-2-oxoethyl]carbamate as a pale brown solid (170 mg, 242%), which was used without further purification in the subsequent reaction (Example 151).

LCMS (Method formate): Retention time 1.35 min, MH⁺=532

### Preparation 155

### 5-{3-[3-(bromoacetyl)-6-methyl-2,3,4,5-tetrahydro-1H-3-benzazepin-7-yl]-1,2,4-oxadiazol-5-yl}-2-[(1-methylethyl)oxy]benzonitrile

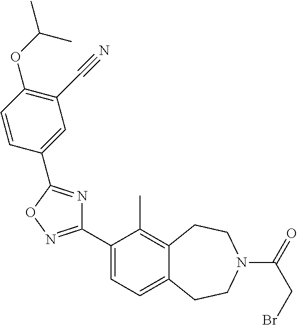

A solution of bromoacetyl bromide (170 μl, 2 mmol) in DCM (1 ml) was added dropwise to a stirred mixture of 2-[(1-methylethyl)oxy]-5-[3-(6-methyl-2,3,4,5-tetrahydro-1H-3-benzazepin-7-yl)-1,2,4-oxadiazol-5-yl]benzonitrile trifluoroacetate (Preparation 134) (1 g, 2 mmol) and DIPEA (1.04 ml, 6 mmol) in DCM (9 ml). The reaction mixture was stirred at room temperature for 2 h. The solvent was evaporated and the residue chromatographed (methanol/DCM, 0-5%) to give 5-{3-[3-(bromoacetyl)-6-methyl-2,3,4,5-tetrahydro-1H-3-benzazepin-7-yl]-1,2,4-oxadiazol-5-yl}-2-[(1-methylethyl)oxy]benzonitrile as a light brown solid (300 mg).

LCMS (Method formate): Retention time 1.26 min, MH⁺=509/511

### Preparation 156

### 2-[7-(5-{3-cyano-4-[(1-methylethyl)oxy]phenyl}-1,2,4-oxadiazol-3-yl)-1,2,4,5-tetrahydro-3H-3-benzazepin-3-yl]-2-methylpropanoic acid trifluoroacetate

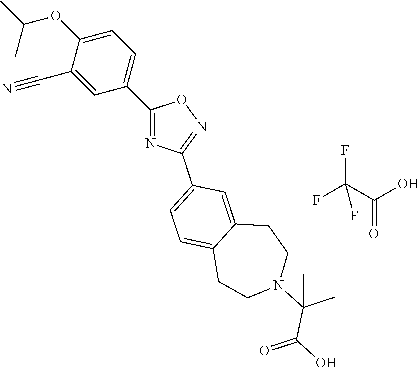

Trifluoroacetic acid (2 ml) was added to a stirred solution of 1,1-dimethylethyl 2-[7-(5-{3-cyano-4-[(1-methylethyl)oxy]phenyl}-1,2,4-oxadiazol-3-yl)-1,2,4,5-tetrahydro-3H-3-benzazepin-3-yl]-2-methylpropanoate (Preparation 157) (120 mg, 0.23 mmol) in DCM (10 ml). The reaction mixture was stirred at room temperature for 24 h. The solvent was evaporated. The residue was re-evaporated from toluene (x2) and the residue triturated with diethyl ether to give 2-[7-(5-{3-cyano-4-[(1-methylethyl)oxy]phenyl}-1,2,4-oxadiazol-3-yl)-1,2,4,5-tetrahydro-3H-3-benzazepin-3-yl]-2-methylpropanoic acid trifluoroacetate as a colourless solid (110 mg).

LCMS (Method formate): Retention time 0.92 min, MH⁺=461

### Preparation 157

### 1,1-dimethylethyl 2-[7-(5-{3-cyano-4-[(1-methylethyl)oxy]phenyl}-1,2,4-oxadiazol-3-yl)-1,2,4,5-tetrahydro-3H-3-benzazepin-3-yl]-2-methylpropanoate

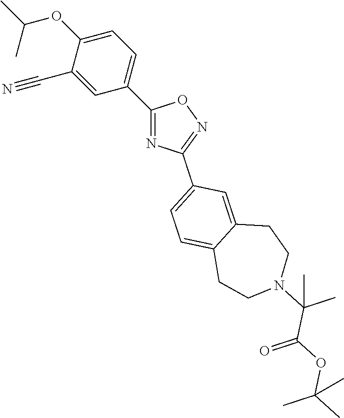

A mixture of 2-[(1-methylethyl)oxy]-5-[3-(2,3,4,5-tetrahydro-1H-3-benzazepin-7-yl)-1,2,4-oxadiazol-5-yl]benzonitrile hydrochloride (Preparation 43) (250 mg, 0.61 mmol), t-butyl 2-bromo-2-methylpropionate (125 μl, 0.67 mmol) and potassum carbonate (250 mg, 1.8 mmol) in acetonitrile (5 ml) was stirred at 50° C. for 24 h. DMF (2 ml) was added and the reaction mixture was heated (microwave) at 140° C. for 6 h. The reaction mixture was diluted with ethyl acetate (20 ml) and washed with water (x2) and brine. The organic phase was dried and evaporated. Chromatography (ethyl acetate/isohexane, 40%) gave 1,1-dimethylethyl 2-[7-(5-{3-cyano-4-[(1-methylethyl)oxy]phenyl}-1,2,4-oxadiazol-3-yl)-1,2,4,5-tetrahydro-3H-3-benzazepin-3-yl]-2-methylpropanoate as a pale yellow oil (120 mg).

LCMS (Method formate): Retention time 1.08 min, MH⁺=517

### Preparation 158

### 2-[7-(5-{3-cyano-4-[(1-methylethyl)oxy]phenyl}-1,2,4-oxadiazol-3-yl)-1,2,4,5-tetrahydro-3H-3-benzazepin-3-yl]propanoic acid trifluoroacetate

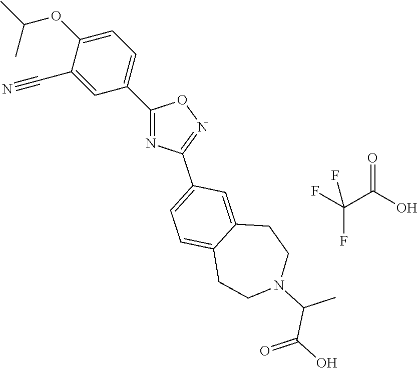

Trifluoroacetic acid (2 ml) was added to a stirred solution of 1,1-dimethylethyl 2-[7-(5-{3-cyano-4-[(1-methylethyl)oxy]phenyl}-1,2,4-oxadiazol-3-yl)-1,2,4,5-tetrahydro-3H-3-benzazepin-3-yl]propanoate (Preparation 159) (250 mg, 0.5 mmol) in DCM (10 ml). The reaction mixture was stirred at room temperature for 24 h. The solvent was evaporated and the residue was re-evaporated from toluene (x2) The residue was triturated with diethyl ether to give 2-[7-(5-{3-cyano-4-[(1-methylethyl)oxy]phenyl}-1,2,4-oxadiazol-3-yl)-1,2,4,5-tetrahydro-3H-3-benzazepin-3-yl]propanoic acid trifluoroacetate as a colourless solid (250 mg).

LCMS (Method formate): Retention time 0.91 min, MH⁺=447

### Preparation 159

### 1,1-dimethylethyl 2-[7-(5-{3-cyano-4-[(1-methylethyl)oxy]phenyl}-1,2,4-oxadiazol-3-yl)-1,2,4,5-tetrahydro-3H-3-benzazepin-3-yl]propanoate

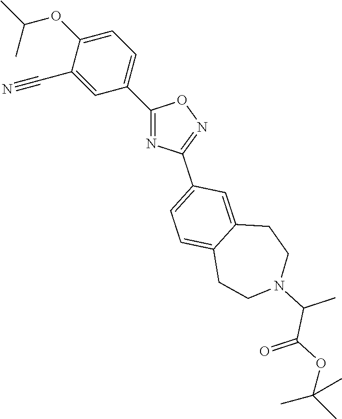

A mixture of 2-[(1-methylethyl)oxy]-5-[3-(2,3,4,5-tetrahydro-1H-3-benzazepin-7-yl)-1,2,4-oxadiazol-5-yl]benzonitrile hydrochloride (Preparation 43) (250 mg, 0.61 mmol), t-butyl 2-bromopropionate (140 mg, 0.67 mmol) and potassum carbonate (250 mg, 1.8 mmol) in acetonitrile (5 ml) was stirred at 50° C. for 24 h. The reaction mixture was cooled and the mixture filtered. The solvent was evaporated from the filtrate and the residue chromatographed (ethyl acetate/isohexane, 20%) to give 1,1-dimethylethyl 2-[7-(5-{3-cyano-4-[(1-methylethyl)oxy]phenyl}-1,2,4-oxadiazol-3-yl)-1,2,4,5-tetrahydro-3H-3-benzazepin-3-yl]propanoate as a colourless oil which solidified (250 mg).

LCMS (Method formate): Retention time 1.14 min, MH⁺=503

### Preparation 160

### (2R)-3-[7-(5-{3-cyano-4-[(1-methylethyl)oxy]phenyl}-1,2,4-oxadiazol-3-yl)-1,2,4,5-tetrahydro-3H-3-benzazepin-3-yl]-2-hydroxypropanoic acid

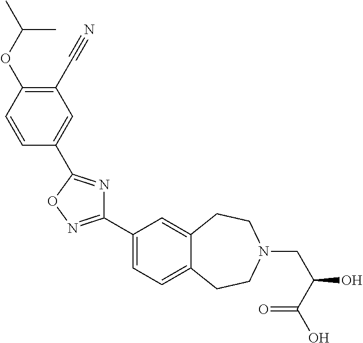

A mixture of methyl (2R)-3-[7-(5-{3-cyano-4-[(1-methylethyl)oxy]phenyl}-1,2,4-oxadiazol-3-yl)-1,2,4,5-tetrahydro-3H-3-benzazepin-3-yl]-2-hydroxypropanoate (Preparation 161) (210 mg), ethanol (2 ml) and sodium hydroxide (2M, 2 ml) was stirred at room temperature for 6 h. The reaction mixture was diluted with water (10 ml) and acidified with glacial acetic acid. The mixture was extracted with ethyl acetate (2×10 ml). The combined extracts were dried and evaporated. Trituration of the residue with diethyl ether gave (2R)-3-[7-(5-{3-cyano-4-[(1-methylethyl)oxy]phenyl}-1,2,4-oxadiazol-3-yl)-1,2,4,5-tetrahydro-3H-3-benzazepin-3-yl]-2-hydroxypropanoic acid as a colourless solid (150 mg).

LCMS (Method formate): Retention time 0.91 min, MH⁺=463

### Preparation 161

### methyl (2R)-3-[7-(5-{3-cyano-4-[(1-methylethyl)oxy]phenyl}-1,2,4-oxadiazol-3-yl)-1,2,4,5-tetrahydro-3H-3-benzazepin-3-yl]-2-hydroxypropanoate

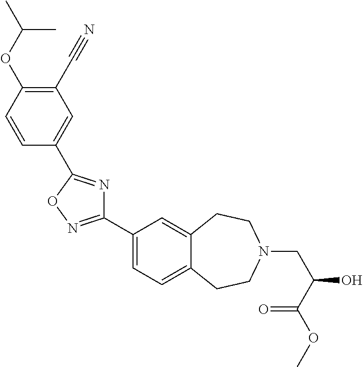

A mixture of 2-[(1-methylethyl)oxy]-5-[3-(2,3,4,5-tetrahydro-1H-3-benzazepin-7-yl)-1,2,4-oxadiazol-5-yl]benzonitrile hydrochloride (Preparation 43) (200 mg, 0.49 mmol), methyl 2-(R)-glycidate (60 mg, 0.59 mmol) and DIPEA (255 μl, 1.5 mmol) in acetonitrile (5 ml) was stirred at 40° C. for 4 days. The reaction mixture was cooled and the solvent evaporated. The residue was chromatographed (methanol/DCM, 0-3%) to give methyl (2R)-3-[7-(5-{3-cyano-4-[(1-methylethyl)oxy]phenyl}-1,2,4-oxadiazol-3-yl)-1,2,4,5-tetrahydro-3H-3-benzazepin-3-yl]-2-hydroxypropanoate as a colourless gum.

LCMS (Method formate): Retention time 0.85 min, MH⁺=477

### Preparation 162

### 2-[(1-methylethyl)oxy]-5-[3-(1,2,3,4-tetrahydro-6-isoquinolinyl)-1,2,4-oxadiazol-5-yl]-3-pyridinecarbonitrile trifluoroacetate

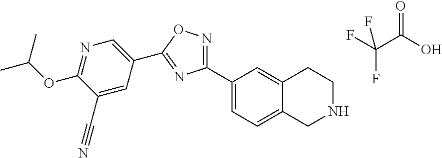

Trifluoroacetic acid (0.52 ml, 6.7 mmol) was added dropwise to a solution of 1,1-dimethylethyl 6-(5-{5-cyano-6-[(1-methylethyl)oxy]-3-pyridinyl}-1,2,4-oxadiazol-3-yl)-3,4-dihydro-2(1H)-isoquinolinecarboxylate (Preparation 163) (620 mg, 1.3 mmol) in DCM (5 ml) at 0° C., the mixture allowed to warm to room temp and stirred for 16 h. The mixture was evaporated and the resulting solid triturated with diethyl ether (2×5 ml) and the solid isolated by filtration to give 2-[(1-methylethyl)oxy]-5-[3-(1,2,3,4-tetrahydro-6-isoquinolinyl)-1,2,4-oxadiazol-5-yl]-3-pyridinecarbonitrile trifluoroacetate as a colourless solid (630 mg).

LCMS (Method formate): Retention time 0.92 min, MH⁺=362

### Preparation 163

### 1,1-dimethylethyl 6-(5-{5-cyano-6-[(1-methylethyl)oxy]-3-pyridinyl}-1,2,4-oxadiazol-3-yl)-3,4-dihydro-2(1H)-isoquinolinecarboxylate

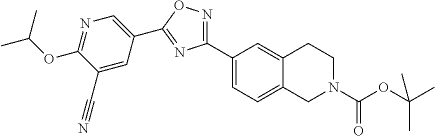

5-Cyano-6-[(1-methylethyl)oxy]-3-pyridinecarbonyl chloride (Preparation 98) (520 mg, 2.3 mmol) was added to a solution of 1,1-dimethylethyl 6-[(hydroxyamino)(imino)methyl]-3,4-dihydro-2(1H)-isoquinolinecarboxylate (Preparation 164) (640 mg, 2.2 mmol) in toluene (8 ml) and pyridine (8 ml) at room temp under nitrogen, the mixture stirred for 20 min at room temperature and then heated at 120° C. for 2 h. The solvent was evaporated and the residue partitioned between water (15 ml) and ethyl acetate (3×15 ml). The combined organic extracts were washed with brine (20 ml) and dried (MgSO₄). The solvent was evaporated and the residue dissolved in DCM and applied to a silica cartridge. The cartridge was eluted with an ethyl acetate/cyclohexane gradient (0-50%). The appropriate fractions were combined and evaporated in vacuo to give 1,1-dimethylethyl 6-(5-{5-cyano-6-[(1-methylethyl)oxy]-3-pyridinyl}-1,2,4-oxadiazol-3-yl)-3,4-dihydro-2(1H)-isoquinolinecarboxylate (690 mg) as a colourless foam.

LCMS (Method formate): Retention time 1.54 min, [2M+H]⁺=923

### Preparation 164

### 1,1-dimethylethyl 6-[(hydroxyamino)(imino)methyl]-3,4-dihydro-2(1H)-isoquinolinecarboxylate

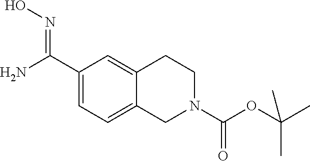

Hydroxylamine hydrochloride (3.50 g, 50 mmol) was added to a suspension of 1,1-dimethylethyl 6-cyano-3,4-dihydro-2(1H)-isoquinolinecarboxylate (Preparation 165) (2.17 g, 8.4 mmol) and sodium hydrogen carbonate (4.23 g, 50 mmol) in ethanol (100 ml) and the mixture heated at 80° C. for 4 h. The cooled mixture was filtered and the filtrate evaporated to give 1,1-dimethylethyl 6-[(hydroxyamino)(imino)methyl]-3,4-dihydro-2(1H)-isoquinolinecarboxylate as a colourless foam (2.78 g).

LCMS (Method formate): Retention time 0.70 min, MH⁺=292

### Preparation 165

### 1,1-dimethylethyl 6-cyano-3,4-dihydro-2(1H)-isoquinolinecarboxylate

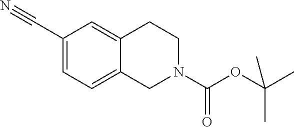

Di-tert-butyl dicarbonate (2.23 ml, 9.6 mmol) was added to a mixture of 1,2,3,4-tetrahydro-6-isoquinolinecarbonitrile (Preparation 166) (1.38 g, 8.7 mmol) and triethylamine (3.65 ml, 26 mmol) in DCM (25 ml) and the mixture was stirred at room temperature for 60 min at 25° C. The solution was washed with water (25 ml) and dried (MgSO₄). The solvent was evaporated and the residue was dissolved in DCM and loaded onto a silica cartridge. The cartridge was eluted with an ethyl acetate/cyclohexane gradient (10-50%). The appropriate fractions were combined and evaporated in vacuo to give 1,1-dimethylethyl 6-cyano-3,4-dihydro-2(1H)-isoquinolinecarboxylate (2.17 g) as a colourless solid.

LCMS (Method formate): Retention time 1.17 min, MH⁺=259

### Preparation 166

### 1,2,3,4-tetrahydro-6-isoquinolinecarbonitrile

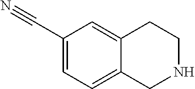

A mixture of 6-bromo-1,2,3,4-tetrahydroisoquinoline (2.86 g, 12 mmol, Allichem LLC), zinc cyanide (1.76 g, 15 mmol) and tetrakis(triphenylphosphine)palladium (0) (1.33 g, 1.2 mmol) in DMF (20 ml) was split between two microwave vials and heated (microwave) for 60 min at 130° C. The mixture was concentrated in vacuo and the residue dissolved in DCM and loaded onto a silica cartridge. The cartridge was eluted with a gradient of 2M ammonia in methanol/DCM (5-10%). The appropriate fractions were combined and evaporated in vacuo to give 1,2,3,4-tetrahydro-6-isoquinolinecarbonitrile (1.41 g) as a colourless solid.

LCMS (Method formate): Retention time 0.36 min, MH⁺=158

### Preparation 167

### 1,1-Dimethylethyl 5-methyl-6-oxo-3,4,6,7,8,8a-hexahydro-2(1H)-isoquinolinecarboxylate

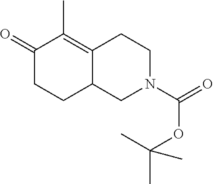

1,1-Dimethylethyl 4-oxo-1-piperidinecarboxylate (70 g, 350 mmol, Aldrich,) and pyrrolidine (43.6 ml, 530 mmol) were dissolved in toluene and the resulting mixture was refluxed under Dean-Stark conditions for 24 h and concentrated in vacuo. The residue was dissolved in anhydrous toluene and treated with hydroquinone (0.40 g) and 1-penten-3-one (29.6 g, 350 mmol). The resulting solution was refluxed for 24 h and diluted with ethyl acetate (300 ml). The mixture was washed with a hydrochloric acid (0.5N, 500 ml) and the aqueous phase extracted with ethyl acetate (300 ml). The combined organic phases were dried (MgSO₄) and concentrated. Purification of the residue by flash chromatography on a silica cartridge (1.5 kg) gave 1,1-dimethylethyl 5-methyl-6-oxo-3,4,6,7,8,8a-hexahydro-2(1H)-isoquinolinecarboxylate (55.2 g, 59.2%) as pale yellow oil which crystallised on standing.

LCMS (Method formate): Retention time 1.04 min, MH⁺=266

1H NMR (CDCl₃): δ H 4.16-4.02 (2H, bm), 3.08-3.01 (1H, m), 2.77-2.71 (1H, m), 2.58-2.49 (3H, m), 2.39-2.26 (2H, m), 2.06-2.00 (1H, m), 1.79 (3H, s), 1.59-1.52 (1H, m), 1.49 (9H, s).

### Preparation 168

### 1,1-Dimethylethyl 6-hydroxy-5-methyl-3,4-dihydro-2(1H)-isoquinolinecarboxylate

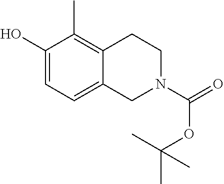

Lithium bis(trimethylsilyl)amide (1M in THF, 246 ml, 250 mmol) was added dropwise to a solution of 1,1-dimethylethyl 5-methyl-6-oxo-3,4,6,7,8,8a-hexahydro-2(1H)-isoquinolinecarboxylate (Preparation 167) (54.4 g, 210 mmol) in THF (200 ml) at −63° C., maintaining a reaction temperature below −60° C. during the addition and the mixture was stirred for 30 min. Chloro(trimethyl)silane (31.4 ml, 250 mmol) was added dropwise and the resulting mixture was stirred for 2 h at −70° C. The reaction was warmed to room temperature over 20 min and diluted with diethyl ether (800 ml). The reaction was added to a saturated sodium carbonate solution and the phases separated. The aqueous phase was extracted with diethyl ether (300 ml) and the combined organic phases were dried (Na₂SO₄) and concentrated in vacuo. The residue was dissolved in acetonitrile (200 ml) and palladium (II) acetate (46.0 g, 210 mmol) was added. The resulting mixture was cooled (water bath) to maintain a reaction temperature below 35° C. and stirred overnight. The reaction was filtered through Celite™ and the residue rinsed with ethyl acetate (3×300 ml). The filtrate was further filtered through a 1″ pad of silica gel and concentrated. The residue was dissolved in ethyl acetate (500 ml), treated with tetrabutylammonium fluoride (1M in THF, 200 ml). The resulting mixture was allowed to stand for 30 min, washed with hydrochloric acid (0.5N, 300 ml) and a 10% sodium thiosulphate solution, dried (MgSO₄) and concentrated. Purification of the residue by flash chromatography on silica gel eluting with an ethyl acetate/cyclohexane gradient (0-60%) gave 1,1-dimethylethyl 6-hydroxy-5-methyl-3,4-dihydro-2(1H)-isoquinolinecarboxylate (29.9 g, 55.4%) as a white solid.

LCMS (Method formate): Retention time 1.07 min, [M−H]=262

1H NMR (CDCl₃): δ H 6.82 (1H, d), 6.66 (1H, d), 4.96 (1H, s), 4.49 (2H, s), 3.64 (2H, t), 2.73 (2H, t), 2.14 (3H, s), 1.48 (9H, s).

### Preparation 168: Alternative Procedure 1,1-dimethylethyl 6-hydroxy-5-methyl-3,4-dihydro-2(1H)-isoquinolinecarboxylate

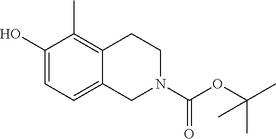

The solution of 1,1-dimethylethyl 5-methyl-6-oxo-3,4,6,7,8,8a-hexahydro-2(1H)-isoquinolinecarboxylate in toluene prepared in the previous experiment (Preparation 172) (˜55 l), was diluted with toluene (115 l) and the solution distilled to a residual volume of ˜65 l. The residue was diluted with anhydrous 2-methyltetrahydrofuran (115 l), mixed and divided into 2 equal portions.

To one portion of this solution under nitrogen were added 2-methyltetrahydrofuran (54 l) and triethylsilylchloride (7.57 kg) and the mixture cooled to <15° C. Lithium-hexamethyldiisilazane (24% in THF, 45.6 kg) was added over 1.5 h maintaining a reaction temperature <20° C. and the reaction maintained at 20±3° C. for 30 min after completion of addition. Water (54 kg) was added to the reaction and the mixture stirred at 20±3° C. for 10 min. The aqueous layer was separated and the organic washed with water (54 kg) at 20±3° C. for 10 min. The organic layer was concentrated by distillation under vacuum to ˜22 l. To the residue was added anhydrous 2-methyltetrahydrofuran (120 l) and the mixture concentrated by distillation under vacuum to ˜30 l before cooling to 20±3° C. To the residue was added anhydrous 2-methyltetrahydrofuran (178 l), palladium (II) acetate (11.5 kg). The catalyst washed in with anhydrous 2-methyltetrahydrofuran (1.7 kg) and the mixture treated with aqueous potassium formate solution (29%, 20.2 kg), maintaining a reaction temperature of 20±5° C. The reaction was stirred at 20±5° C. for 2 h and then held over the weekend. To the reaction was added tetrabutylammonium fluoride (1M in THF, 20.4 kg) and the reaction stirred at 20±3° C. for ˜1.5 h and then filtered. The solid was washed with 2-methyltetrahydrofuran (2×24 l). The aqueous phase was separated from the combined filtrate and washings and the organic washed with water (60 kg) then aqueous sodium chloride solution (1%, 60 kg). The organic phase was concentrated to ˜36 l by distillation under vacuum, the cooled residue diluted with 2-methyltetrahydrofuran (180 l) and redistilled to a residual volume of 361. A slurry of 1,1-dimethylethyl 6-hydroxy-5-methyl-3,4-dihydro-2(1H)-isoquinolinecarboxylate (12 g) in heptane (0.5 l) was added to the residual solution and the mixture stirred at 20±3° C. for 1.5 h. A further portion of slurried 1,1-dimethylethyl 6-hydroxy-5-methyl-3,4-dihydro-2(1H)-isoquinolinecarboxylate (12 g) in heptane (0.5 l) was added, the mixture stirred for 5 min and heptane (116 kg) added to the mixture over 2 h at 20±3° C. The mixture was concentrated to 361 under vacuum, cooled and the residue diluted with heptane (144 l) over 30 min. The mixture was filtered, the solid washed with heptane (2×36 l) and dried under vacuum at 45±5° C. to give 1,1-dimethylethyl 6-hydroxy-5-methyl-3,4-dihydro-2(1H)-isoquinolinecarboxylate (5.7 kg).

1H NMR (CDCl₃): δ H 6.80 (1H, d), 6.66 (1H, d), 5.35 (1H, bs), 4.49 (2H, s), 3.64 (2H, t), 2.72 (2H, t), 2.14 (3H, s), 1.49 (9H, s).

### Preparation 169

### 3-Cyano-4-[(1-methylethyl)oxy]benzoyl chloride

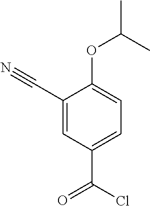

Oxalyl chloride (6.39 ml, 73 mmol) was added to a solution of 3-cyano-4-[(1-methylethyl)oxy]benzoic acid (10.7 g, 52 mmol, Biopharma Inc.) in DCM (100 ml) followed by the addition of DMF (0.044 ml, 0.57 mmol) and the mixture stirred at room temperature for 4 h. The reaction mixture was filtered and concentrated. The residue was co-evaporated with cyclohexane (2×50 ml) to give 3-cyano-4-[(1-methylethyl)oxy]benzoyl chloride (Preparation 169) (11.7 g, 100%) as a pale yellow oil which solidified on standing.

1H NMR (CDCl₃): δ H 8.36 (1H, d), 8.26 (1H, dd), 7.06 (1H, d), 4.81 (1H, m), 1.47 (6H, d).

### Preparation 170

### 1,1-dimethylethyl 6-cyano-5-methyl-3,4-dihydro-2(1H)-isoquinolinecarboxylate

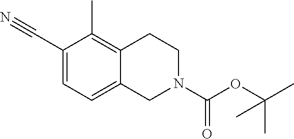

The solution and reactor wash solutions of 1,1-dimethylethyl 5-methyl-6-{[(trifluoromethyl)sulfonyl]oxy}-3,4-dihydro-2(1H)-isoquinolinecarboxylate in ethyl acetate from Preparation 171 were combined and reduced to 31 l under vacuum distillation and then cooled to 20±3° C. Ethyl acetate (47 l) was added and the vacuum distillation repeated. DMF (74 kg) was added to the cooled residue and the mixture again distilled to 31 l maintaining a temperature <75° C. DMF (74 kg) was added to the cooled mixture and the solution divided into 2 equal portions.

One portion of DMF solution was purged with nitrogen (x3) and warmed to 90±5° C. for 2.5 h, diluted with DMF (5.3 kg) and the nitrogen purge repeated (x3). The mixture was cooled to 70±5° C., zinc cyanide (3.01 kg) and tetrakis(triphenylphosphine)palladium (2.26 kg) added and the mixture purged with nitrogen (x6). The reaction was warmed to 105±3° C. under nitrogen and maintained at this temperature for 15 min, before heating at 120±3° C. for 5 h. The reaction was cooled to 50±5° C. and purged with nitrogen (x6), then further cooled to 20±5° C. Toluene (78 l) and water (78 l) were added to the reaction, stirred for 45 min, the mixture filtered through Celite™ (6.5 kg) and the residues washed with toluene (55 l). The combined filtrate and washings were stirred for 15 min, the aqueous removed, the organic washed sequentially with brine (86.4 kg of a solution of sodium chloride (25.9 kg) in water (149 l)) over 15 min and water (78 kg) over 15 min to give 1,1-dimethylethyl 6-cyano-5-methyl-3,4-dihydro-2(1H)-isoquinolinecarboxylate as a solution in toluene.

The second portion of DMF solution was diluted with DMF (5.3 kg) purged with nitrogen (x3) and warmed to 90±5° C. for 2 h10 min. The mixture was cooled to 70±5° C., zinc cyanide (3.01 kg) and tetrakis(triphenylphosphine) palladium (2.26 kg) added and the mixture purged with nitrogen (x6). The reaction was warmed to 105±3° C. under nitrogen and maintained at this temperature for 15 min, before heating at 120±3° C. for 5 h. The reaction was cooled to 50±5° C. and purged with nitrogen (x6), then further cooled to 20±5° C. Toluene (78 l) and water (78 l) were added to the reaction, stirred for 45 min, the mixture filtered through Celite™ (6.5 kg) and the residues washed with toluene (55 l). The combined filtrate and washings were stirred for 19 min, the aqueous removed, the organic washed sequentially with brine (remainder of the solution of sodium chloride (25.9 kg) in water (149 l)) over 15 min and water (78 kg) over 16 min to give 1,1-dimethylethyl 6-cyano-5-methyl-3,4-dihydro-2(1H)-isoquinolinecarboxylate as a solution in toluene. The 2 solutions of the desired product in toluene were combined, warmed to 40±3° C. and cooled to 20±3° C. This solution was used directly in the subsequent reaction (Preparation 23 alternative procedure).

### Preparation 171

### 1,1-dimethylethyl 5-methyl-6-{[(trifluoromethyl)sulfonyl]oxy}-3,4-dihydro-2(1H)-isoquinoline carboxylate

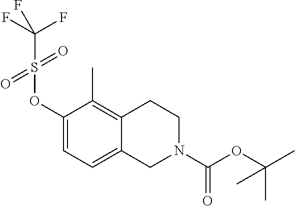

To a solution of 1,1-dimethylethyl 6-hydroxy-5-methyl-3,4-dihydro-2(1H)-isoquinolinecarboxylate (Preparation 168) (11.05 kg) in pyridine (28 kg) at 0±3° C. was added triflic anhydride (14.3 kg) maintaining a reaction temperature of 5-15° C. Ethyl acetate (111 l) and water (55 l) were added to the reaction, the aqueous extracted with ethyl acetate (33 l) and the combined organic phases washed at 40±3° C. with hydrochloric acid (1 M, 68 kg), then hydrochloric acid (1 M, 2×66 kg). The organic was further washed at 35±3° C. with aqueous sodium carbonate solution (2.76 kg in water 52 l) and water (55 kg) to give 1,1-dimethylethyl 5-methyl-6-{[(trifluoromethyl)sulfonyl]oxy}-3,4-dihydro-2(1H)-isoquinolinecarboxylate as a solution in ethyl acetate (99.1 kg). The reactor vessel was washed with ethyl acetate (10 l) and this solution of 1,1-dimethylethyl 5-methyl-6-{[(trifluoromethyl)sulfonyl]oxy}-3,4-dihydro-2(1H)-isoquinolinecarboxylate also retained for use in the subsequent experiment (Preparation 168 alternative procedure).

### Preparation 172

### 1,1-dimethylethyl 5-methyl-6-oxo-3,4,6,7,8,8a-hexahydro-2(1H)-isoquinolinecarboxylate

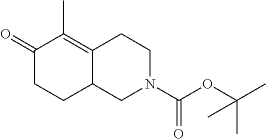

Under nitrogen, 1,1-dimethylethyl 4-oxo-1-piperidinecarboxylate (19.76 kg) was added to toluene (87 l) and washed in with toluene (2 kg), the resulting solution was treated with pyrrolidine (10.9 kg) and the reaction heated at reflux for 2 h under Dean-Stark conditions and then cooled to 60±3° C. The reaction mixture was heated to boiling and concentrated by distillation (70 kg volatiles removed). The residue was cooled to 20±3° C., toluene (70 kg) added followed by a slurry of hydroquinone (64 g) in toluene (0.3 l). Ethyl vinyl ketone (8.3 kg) was added maintaining a reaction temperature of <40° C. and once the exotherm had subsided the mixture heated to 40±3° C. for 1 h. The material was transferred to a second reactor vessel, washing with toluene (5 l) and the reaction heated at 105±3° C. for 12 h, cooled to 55±3° C. and then heated at 105±3° C. for a further 6 h. The reaction was cooled to 20±3° C., washed with a solution of ammonium chloride (28.4 kg) in water (75 l), then water (96 kg). After standing overnight the toluene solution was concentrated by distillation to ca 551 to give a toluene solution of 1,1-dimethylethyl 5-methyl-6-oxo-3,4,6,7,8,8a-hexahydro-2(1H)-isoquinolinecarboxylate. A sample (95.45 g) was removed from this solution and the remainder used directly in the subsequent reaction (Preparation 168 alternative procedure).

The sample (95.45 g) was reduced to dryness in vacuo and the residue further dried at ˜40° C. over a weekend to give impure 1,1-dimethylethyl 5-methyl-6-oxo-3,4,6,7,8,8a-hexahydro-2(1H)-isoquinolinecarboxylate as a yellow/orange viscous oil (43.65 g) which slowly solidified on standing.

### Preparation 173

### 5-{3-[2-(2,2-dimethyl-1,3-dioxan-5-yl)-1,2,3,4-tetrahydro-6-isoquinolinyl]-1,2,4-oxadiazol-5-yl}-2-[(1-methylethyl)oxy]-3-pyridinecarbonitrile

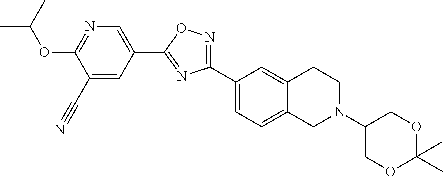

Sodium triacetoxyborohydride (334 mg, 1.6 mmol) was added portionwise to a stirred supension of 2-[(1-methylethyl)oxy]-5-[3-(1,2,3,4-tetrahydro-6-isoquinolinyl)-1,2,4-oxadiazol-5-yl]-3-pyridinecarbonitrile trifluoroacetate (Preparation 162) (150 mg, 0.31 mmol) and 2,2-dimethyl-1,3-dioxan-5-one (205 mg, 1.6 mmol) in dry DCM (5 ml). The reaction mixture was stirred at room temperature for 24 h. Saturated sodium hydrogen carbonate (5 ml) was added carefully and the mixture stirred vigorously for 30 min. The organic phase was separated. The aqueous phase was extracted with DCM (2×10 ml). The combined organics were dried and evaporated. Chromatography (methanol/DCM, 0-5%) gave 5-{3-[2-(2,2-dimethyl-1,3-dioxan-5-yl)-1,2,3,4-tetrahydro-6-isoquinolinyl]-1,2,4-oxadiazol-5-yl}-2-[(1-methylethyl)oxy]-3-pyridinecarbonitrile as a pale yellow solid.

LCMS (Method formate): Retention time 0.98 min, MH⁺=476

### Preparation 174

### [6-(5-{3-cyano-4-[(1-methylethyl)oxy]phenyl}-1,2,4-oxadiazol-3-yl)-3,4-dihydro-2(1H)-isoquinolinyl]acetic acid trifluoroacetate

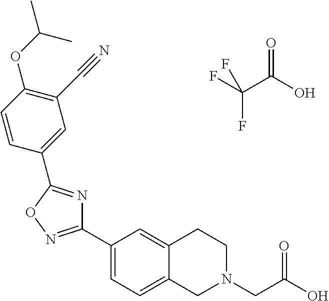

Trifluoroacetic acid (2 ml) was added to a stirred solution of 1,1-dimethylethyl [6-(5-{3-cyano-4-[(1-methylethyl)oxy]phenyl}-1,2,4-oxadiazol-3-yl)-3,4-dihydro-2(1H)-isoquinolinyl]acetate (Preparation 175) (710 mg, 1.5 mmol) in DCM (8 ml). The reaction mixture was stirred at room temperature overnight. The solvent was evaporated. The residue was re-evaporated from toluene (x2). Trituration with diethyl ether gave [6-(5-{3-cyano-4-[(1-methylethyl)oxy]phenyl}-1,2,4-oxadiazol-3-yl)-3,4-dihydro-2(1H)-isoquinolinyl]acetic acid trifluoroacetate as a colourless solid (690 mg).

LCMS (Method formate): Retention time 0.85 min, MH⁺=419

### Preparation 175

### 1,1-dimethylethyl [6-(5-{3-cyano-4-[(1-methylethyl)oxy]phenyl}-1,2,4-oxadiazol-3-yl)-3,4-dihydro-2(1H)-isoquinolinyl]acetate

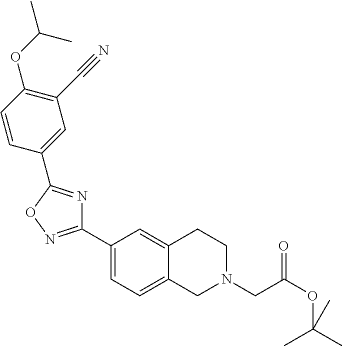

A mixture of 2-[(1-methylethyl)oxy]-5-[3-(1,2,3,4-tetrahydro-6-isoquinolinyl)-1,2,4-oxadiazol-5-yl]benzonitrile trifluoroacetate (Preparation 25) (950 mg, 2 mmol), potassium carbonate (830 mg, 6 mmol) and t-butyl bromoacetate (0.31 ml, 2.1 mmol) in acetonitrile (25 ml) was stirred at room temperature overnight. The reaction mixture was diluted with ethyl acetate (15 ml) and washed with water (2×10 ml). The organic phase was separated, dried and evaporated. Chromatography (ethyl acetate/hexane, 25%) gave 1,1-dimethylethyl [6-(5-{3-cyano-4-[(1-methylethyl)oxy]phenyl}-1,2,4-oxadiazol-3-yl)-3,4-dihydro-2(1H)-isoquinolinyl]acetate as a colourless oil which solidified (710 mg).

LCMS (Method formate): Retention time 1.09 min, MH⁺=475

### Preparation 176

### [6-(5-{5-cyano-6-[(1-methylethyl)oxy]-3-pyridinyl}-1,2,4-oxadiazol-3-yl)-5-methyl-3,4-dihydro-2(1H)-isoquinolinyl]acetic acid trifluororacetate

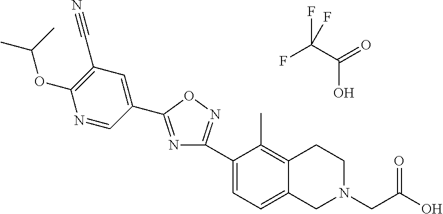

Trifluoroacetic acid (2 ml) was added slowly to a stirred solution of 1,1-dimethylethyl [6-(5-{5-cyano-6-[(1-methylethyl)oxy]-3-pyridinyl}-1,2,4-oxadiazol-3-yl)-5-methyl-3,4-dihydro-2(1H)-isoquinolinyl]acetate (Preparation 177) (480 mg, 0.98 mmol) in DCM (8 ml). The reaction mixture was stirred at room temperature overnight. The solvent was evaporated and the residue re-evaporated from toluene (x2). The residue was triturated with diethyl ether to give [6-(5-{5-cyano-6-[(1-methylethyl)oxy]-3-pyridinyl}-1,2,4-oxadiazol-3-yl)-5-methyl-3,4-dihydro-2(1H)-isoquinolinyl]acetic acid trifluororacetate as a colourless solid (370 mg).

LCMS (Method formate): Retention time 0.92 min, MH⁺=434

### Preparation 177

### 1,1-dimethylethyl [6-(5-{5-cyano-6-[(1-methylethyl)oxy]-3-pyridinyl}-1,2,4-oxadiazol-3-yl)-5-methyl-3,4-dihydro-2(1H)-isoquinolinyl]acetate

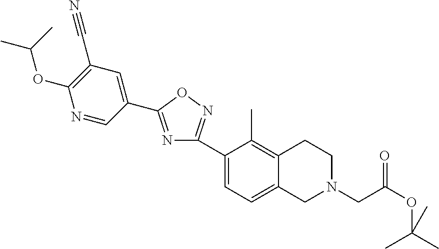

A mixture of 2-[(1-methylethyl)oxy]-5-[3-(5-methyl-1,2,3,4-tetrahydro-6-isoquinolinyl)-1,2,4-oxadiazol-5-yl]-3-pyridinecarbonitrile trifluoroacetate (Preparation 3) (490 mg, 1 mmol), t-butyl bromoacetate (155 μl, 1.1 mmol) and potassium carbonate (415 mg, 3 mmol) in acetonitrile (10 ml) was stirred at room temperature for 24 h. The reaction mixture was diluted with ethyl acetate (20 ml) and washed with water and brine. The organic phase was dried and evaporated. Chromatography (ethyl acetate/hexane, 25%) gave 1,1-dimethylethyl [6-(5-{5-cyano-6-[(1-methylethyl)oxy]-3-pyridinyl}-1,2,4-oxadiazol-3-yl)-5-methyl-3,4-dihydro-2(1H)-isoquinolinyl]acetate as a colourless oil which solidified (480 mg).

LCMS (Method formate): Retention time 1.10 min, MH⁺=490

### Preparation 178

### 2-[(1-methylethyl)amino]-5-[3-(6-methyl-2,3,4,5-tetrahydro-1H-3-benzazepin-7-yl)-1,2,4-oxadiazol-5-yl]-3-pyridinecarbonitrile hydrochloride

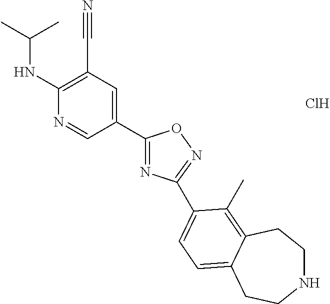

1,1-Dimethylethyl 7-(5-{5-cyano-6-[(1-methylethyl)amino]-3-pyridinyl}-1,2,4-oxadiazol-3-yl)-6-methyl-1,2,4,5-tetrahydro-3H-3-benzazepine-3-carboxylate (Preparation 179) (1.3 g, 2.7 mmol) was suspended in 1,4-dioxane (5 ml) and treated with hydrogen chloride in 1,4-dioxane (4M, 10 ml). The reaction mixture was stirred at room temperature for 7 h. The reaction mixture was diluted with diethyl ether (70 ml). The solid was isolated by filtration, washed with diethyl ether and dried to give 2-[(1-methylethyl)amino]-5-[3-(6-methyl-2,3,4,5-tetrahydro-1H-3-benzazepin-7-yl)-1,2,4-oxadiazol-5-yl]-3-pyridinecarbonitrile hydrochloride as a colourless solid (1.05 g).

LCMS (Method formate): Retention time 0.83 min, MH⁺=389

### Preparation 179

### 1,1-Dimethylethyl 7-(5-{5-cyano-6-[(1-methylethyl)amino]-3-pyridinyl}-1,2,4-oxadiazol-3-yl)-6-methyl-1,2,4,5-tetrahydro-3H-3-benzazepine-3-carboxylate

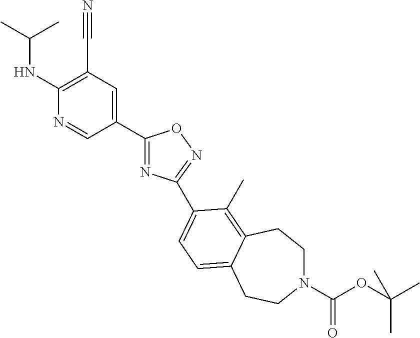

A mixture of 5-cyano-6-[(1-methylethyl)amino]-3-pyridinecarboxylic acid (Preparation 122) (1 g, 4.9 mmol), N-ethylmorpholine (1.23 ml, 9.8 mmol), HATU (2.22 g, 5.9 mmol) and 1,1-dimethylethyl 7-[(hydroxyamino)(imino)methyl]-6-methyl-1,2,4,5-tetrahydro-3H-3-benzazepine-3-carboxylate (Preparation 136) (1.87 g, 5.9 mmol) in DMF (10 ml) was stirred at room temperature for 4 h. The reaction mixture was diluted with ethyl acetate (50 ml) and washed with saturated sodium hydrogen carbonate, water and brine. The organic phase was dried and evaporated. The residue was dissolved in toluene (40 ml) and pyridine (10 ml) and the mixture refluxed for 2 h. The reaction mixture was cooled to room temperature and the solvent evaporated. Chromatography (ethyl acetate/hexane, 20%) gave 1,1-dimethylethyl 7-(5-{5-cyano-6-[(1-methylethyl)amino]-3-pyridinyl}-1,2,4-oxadiazol-3-yl)-6-methyl-1,2,4,5-tetrahydro-3H-3-benzazepine-3-carboxylate as a colourless solid (1.31 g).

LCMS (Method formate): Retention time 1.51 min, MH⁺=489

### Preparation 180

### 2-[(1-methylethyl)amino]-5-[3-(5-methyl-1,2,3,4-tetrahydro-6-isoquinolinyl)-1,2,4-oxadiazol-5-yl]-3-pyridinecarbonitrile hydrochloride

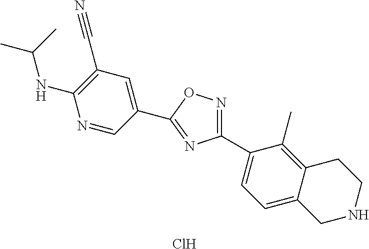

To a solution of 1,1-dimethylethyl 6-(5-{5-cyano-6-[(1-methylethyl)amino]-3-pyridinyl}-1,2,4-oxadiazol-3-yl)-5-methyl-3,4-dihydro-2(1H)-isoquinolinecarboxylate (Preparation 181) (519 mg, 1.1 mmol) in 1,4-dioxane (4 ml) at room temperature under nitrogen was slowly added hydrogen chloride in 1,4-dioxane (4N, 6 ml, 24 mmol) and the resulting mixture was stirred at this temperature for ca 6 h and then concentrated in vacuo. The solid residue was co-evaporated with diethyl ether and dried under vacuum to give 2-[(1-methylethyl)amino]-5-[3-(5-methyl-1,2,3,4-tetrahydro-6-isoquinolinyl)-1,2,4-oxadiazol-5-yl]-3-pyridinecarbonitrile hydrochloride (448 mg, 100%) as a white solid.

LCMS (Method HpH): Retention time 1.22 min, MH⁺=375 (weak)

### Preparation 181

### 1,1-dimethylethyl 6-(5-{5-cyano-6-[(1-methylethyl)amino]-3-pyridinyl}-1,2,4-oxadiazol-3-yl)-5-methyl-3,4-dihydro-2(1H)-isoquinolinecarboxylate

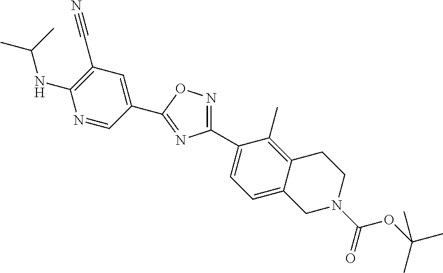

To a suspension of 5-cyano-6-[(1-methylethyl)amino]-3-pyridinecarboxylic acid (Preparation 122) (500 mg, 2.4 mmol) in DCM (15 ml) was added oxalyl chloride (0.277 ml, 3.2 mmol) then DMF (10 μl, 0.12 mmol) and the resulting mixture was stirred 1 h at room temperature. The reaction was concentrated in vacuo and the residue was co-evaporated toluene (x2) then dried for 1 h under vacuum. The residue was added slowly to a solution of 1,1-dimethylethyl 6-[(hydroxyamino)(imino)methyl]-5-methyl-3,4-dihydro-2(1H)-isoquinolinecarboxylate (Preparation 23) (670 mg, 2.2 mmol) in toluene (5 ml) and pyridine (3 ml). After 60 min, the mixture was heated at 120° C. for 4 h and was then cooled to room temperature. Most of the solvent was evaporated in vacuo and the residue partitioned between ethyl acetate and hydrochloric acid (2N). The aqueous was extracted and the combined organic phases washed with saturated sodium hydrogen carbonate, then brine, dried (MgSO₄) and concentrated in vacuo. Purification of the residue by chromatography, eluting with an ethyl acetate/cyclohexane gradient gave 1,1-dimethylethyl 6-(5-{5-cyano-6-[(1-methylethyl)amino]-3-pyridinyl}-1,2,4-oxadiazol-3-yl)-5-methyl-3,4-dihydro-2(1H)-isoquinolinecarboxylate (519 mg, 44.9%) as a pale yellow foam.

LCMS (Method HpH): Retention time 1.55 min, [2M+H]⁺=949

### Preparation 182

### 5-{3-[2-(2,2-dimethyl-1,3-dioxan-5-yl)-1,2,3,4-tetrahydro-5-isoquinolinyl]-1,2,4-oxadiazol-5-yl}-2-[(1-methylethyl)oxy]benzonitrile

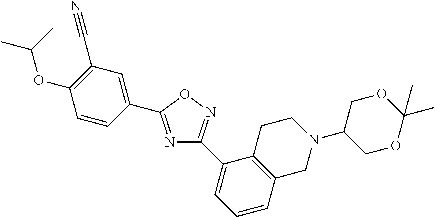

To a stirred suspension of 2-[(1-methylethyl)oxy]-5-[3-(1,2,3,4-tetrahydro-5-isoquinolinyl)-1,2,4-oxadiazol-5-yl]benzonitrile trifluoroacetate (Preparation 91) (400 mg, 0.84 mmol) and 2,2-dimethyl-1,3-dioxan-5-one (0.50 ml, 4.2 mmol) in DCM (15 ml), was added sodium triacetoxyborohydride (536 mg, 2.5 mmol). Saturated sodium hydrogen carbonate (5 ml) was added to the reaction mixture which was stirred vigourously for 10 min. The reaction mixture was extracted using water (20 ml) and DCM (3×20 ml). The combined organic phases were dried (hydrophobic frit) and evaporated under reduced pressure. The residual oil was dissolved in DCM and loaded onto a silica cartridge (10 g), which was eluted with a gradient of methanol/DCM (0-5%). The appropriate fractions were combined and evaporated under vacuum to give the required product 5-{3-[2-(2,2-dimethyl-1,3-dioxan-5-yl)-1,2,3,4-tetrahydro-5-isoquinolinyl]-1,2,4-oxadiazol-5-yl}-2-[(1-methylethyl)oxy]benzonitrile (463 mg, 116%) as a yellow oil.

LCMS (Method formate): Retention time 0.94 min, MH⁺=475

### Preparation 183

### [5-(5-{3-cyano-4-[(1-methylethyl)oxy]phenyl}-1,2,4-oxadiazol-3-yl)-3,4-dihydro-2(1H)-isoquinolinyl]acetic acid trifluoroacetate

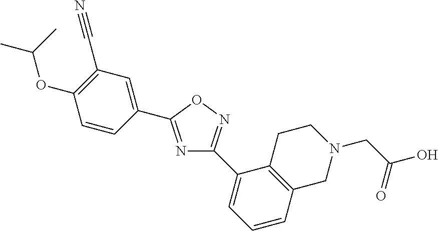

To a stirred solution of 1,1-dimethylethyl [5-(5-{3-cyano-4-[(1-methylethyl)oxy]phenyl}-1,2,4-oxadiazol-3-yl)-3,4-dihydro-2(1H)-isoquinolinyl]acetate (Preparation 184) (604 mg, 1.3 mmol) in DCM (7 ml), was added trifluoroacetic acid (1.7 ml). The solution was stirred overnight. The solvent was evaporated under reduced pressure to give a brown oil which was re-evaporated from toluene (2×10 ml). The resulting oil was triturated with diethyl ether to give [5-(5-{3-cyano-4-[(1-methylethyl)oxy]phenyl}-1,2,4-oxadiazol-3-yl)-3,4-dihydro-2(1H)-isoquinolinyl]acetic acid trifluoroacetate as a yellow solid (500 mg).

LCMS (Method formate): Retention time 0.87 min, MH⁺=419

### Preparation 184

### 1,1-dimethylethyl [5-(5-{3-cyano-4-[(1-methylethyl)oxy]phenyl}-1,2,4-oxadiazol-3-yl)-3,4-dihydro-2(1H)-isoquinolinyl]acetate

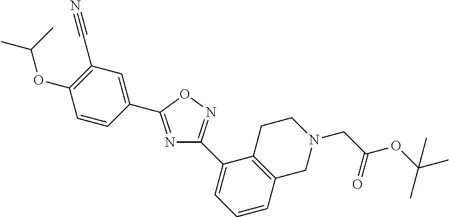

To a stirred solution of 2-[(1-methylethyl)oxy]-5-[3-(1,2,3,4-tetrahydro-5-isoquinolinyl)-1,2,4-oxadiazol-5-yl]benzonitrile trifluoroacetate (Preparation 91) (486 mg, 1.0 mmol) in acetonitrile (13 ml) was added, potassium carbonate (425 mg, 3.1 mmol) and 1,1-dimethylethyl bromoacetate (220 mg, 0.17 mmol). The solution was stirred for 3 h. The reaction mixture was partitioned between ethyl acetate (3×20 ml) and water (20 ml) and the organic dried (hydrophobic frit). The resultant solution was evaporated under reduced pressure. The residual oil was dissolved in DCM, loaded onto a silica cartridge (25 g) and the cartridge eluted with a methanol/DCM gradient (0-5%). The appropriate fractions were combined and evaporated under vacuum to give the required product 1,1-dimethylethyl [5-(5-{3-cyano-4-[(1-methylethyl)oxy]phenyl}-1,2,4-oxadiazol-3-yl)-3,4-dihydro-2(1H)-isoquinolinyl]acetate (60 mg, 1.27 mmol, 124%) as a yellow solid.

LCMS (Method formate): Retention time 1.04 min, MH⁺=475

### Preparation 185

### 5-{5-[2-(2,2-dimethyl-1,3-dioxan-5-yl)-5-methyl-1,2,3,4-tetrahydro-6-isoquinolinyl]-1,3,4-thiadiazol-2-yl}-2-[(1-methylethyl)oxy]benzonitrile

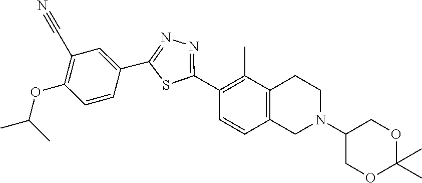

To a solution of 2-[(1-methylethyl)oxy]-5-[3-(5-methyl-1,2,3,4-tetrahydro-6-isoquinolinyl)-1,3,4-thiadiazol-2-yl]benzonitrile trifluoroacetate (Preparation 34) (795 mg, 1.6 mmol) and 2,2-dimethyl-1,3-dioxan-5-one (0.56 ml, 4.7 mmol) in DCM (20 m) was added sodium triacetoxyborohydride (1.67 g, 7.9 mmol) portionwise. The resulting mixture was stirred for 18 h at room temperature. Saturated aqueous sodium hydrogen carbonate was added and the mixture was stirred at room temperature for 15 min. The layers were separated and the aqueous extracted with DCM (x3). The combined organic layers were washed with brine, dried (MgSO₄), filtered and concentrated under reduced pressure. The crude compound was purified by flash chromatography on a silica cartridge (25 g), eluting with a methanol/DCM gradient (2-5%). The appropriate fractions were combined and concentrated under reduced pressure to give 5-{5-[2-(2,2-dimethyl-1,3-dioxan-5-yl)-5-methyl-1,2,3,4-tetrahydro-6-isoquinolinyl]-1,3,4-thiadiazol-2-yl}-2-[(1-methylethyl)oxy]benzonitrile (824 mg) as a yellow solid. This material was used without further purification in the subsequent reaction (Example 184)

LCMS (Method formate): Retention time 0.89 min, MH⁺=505 (weak)

### Preparation 186

### 1,1-dimethylethyl [7-(5-{3-cyano-4-[(1-methylethyl)oxy]phenyl}-1,2,4-oxadiazol-3-yl)-1,2,4,5-tetrahydro-3H-3-benzazepin-3-yl]acetate

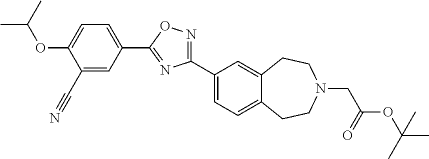

A mixture of 2-[(1-methylethyl)oxy]-5-[3-(2,3,4,5-tetrahydro-1H-3-benzazepin-7-yl)-1,2,4-oxadiazol-5-yl]benzonitrile hydrochloride (Preparation 43) (1 g, 2.4 mmol), t-butyl bromoacetate (396 μl, 2.7 mmol) and potassium carbonate (841 mg, 6.1 mmol) in acetonitrile (10 ml) was stirred at room temperature overnight. The reaction mixture was diluted with acetonitrile (10 ml) and filtered. The solvent was evaporated from the filtrate. The residue was chromatographed (ethyl acetate/isohexane, 20%) to give 1,1-dimethylethyl [7-(5-{3-cyano-4-[(1-methylethyl)oxy]phenyl}-1,2,4-oxadiazol-3-yl)-1,2,4,5-tetrahydro-3H-3-benzazepin-3-yl]acetate as a colourless oil which solidified (912 mg).

LCMS (Method formate): Retention time 1.02 min, MH⁺=489

### Preparation 187

### 5-{3-[3-(bromoacetyl)-8-methyl-2,3,4,5-tetrahydro-1H-3-benzazepin-7-yl]-1,2,4-oxadiazol-5-yl}-2-[(1-methylethyl)oxy]benzonitrile

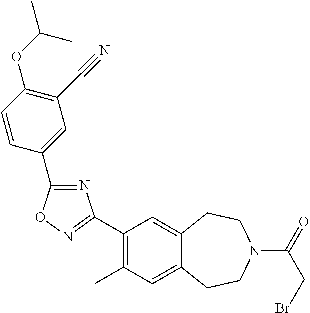

Bromoacetyl bromide (208 mg, 1.0 mmol) was added dropwise to a stirred solution of 2-[(1-methylethyl)oxy]-5-[3-(8-methyl-2,3,4,5-tetrahydro-1H-3-benzazepin-7-yl)-1,2,4-oxadiazol-5-yl]benzonitrile trifluoroacetate (Preparation 14) (400 mg) and diisopropylethylamine (450 μl, 2.6 mmol) in DCM (10 ml). The reaction mixture was stirred at room temperature for 2 h. The solvent was evaporated and the residue chromatographed (ethyl acetate/isohexane, 5-20%) to give 5-{3-[3-(bromoacetyl)-8-methyl-2,3,4,5-tetrahydro-1H-3-benzazepin-7-yl]-1,2,4-oxadiazol-5-yl}-2-[(1-methylethyl)oxy]benzonitrile (140 mg) as a pale yellow solid.

LCMS (Method formate): Retention time 1.36 min, MH⁺=509/511

### Preparation 188

### 1,1-dimethylethyl {2-[7-(5-{3-cyano-4-[(2,2,2-trifluoroethyl)oxy]phenyl}-1,2,4-oxadiazol-3-yl)-1,2,4,5-tetrahydro-3H-3-benzazepin-3-yl]-2-oxoethyl}carbamate

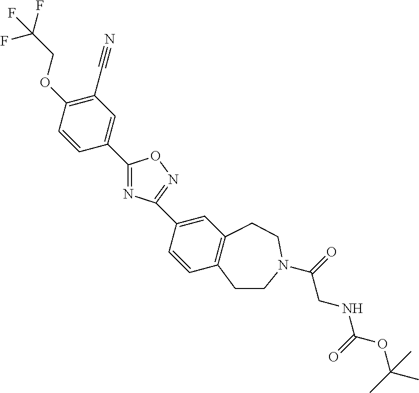

A mixture of N-Bocglycine (60 mg, 0.34 mmol), N-ethylmorpholine (86 μl, 0.68 mmol), hydroxybenzotriazole hydrate (62 mg, 0.41 mmol), EDC (78 mg, 0.41 mmol) and 5-[3-(2,3,4,5-tetrahydro-1H-3-benzazepin-7-yl)-1,2,4-oxadiazol-5-yl]-2-[(2,2,2-trifluoroethyl)oxy]benzonitrile hydrochloride (Preparation 6) (185 mg, 0.41 mmol) in DMF (3 ml) was stirred at room temperature for 6 h. The reaction mixture was partitioned between ethyl acetate (20 ml) and saturated sodium hydrogen carbonate (20 ml). The organic phase was separated, washed with water and brine, dried and evaporated. The residue was chromatographed (methanol/DCM, 0-4%) to give 1,1-dimethylethyl {2-[7-(5-{3-cyano-4-[(2,2,2-trifluoroethyl)oxy]phenyl}-1,2,4-oxadiazol-3-yl)-1,2,4,5-tetrahydro-3H-3-benzazepin-3-yl]-2-oxoethyl}carbamate (163 mg) as a colourless oil.

LCMS (Method formate): Retention time 1.24 min, MH⁺=572

### Preparation 189

### methyl [6-(5-{3-cyano-4-[(1-methylethyl)oxy]phenyl}-1,2,4-oxadiazol-3-yl)-5-methyl-3,4-dihydro-2(1H)-isoquinolinyl]acetate

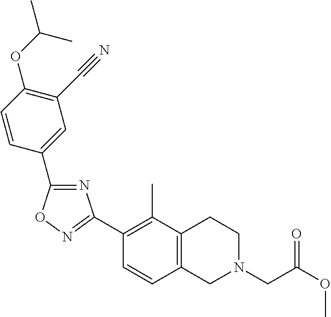

Methyl bromoacetate (377 μl, 4.1 mmol) was added to a stirred mixture of 2-[(1-methylethyl)oxy]-5-[3-(5-methyl-1,2,3,4-tetrahydro-6-isoquinolinyl)-1,2,4-oxadiazol-5-yl]benzonitrile trifluoroacetate (Preparation 25) (2 g, 4.1 mmol) and potassium carbonate (1.42 g, 10 mmol) in dry acetonitrile (30 ml). The reaction mixture was stirred at 50° C. for 1 h. The reaction mixture was cooled and the solvent evaporated. The residue was dissolved in ethyl acetate (50 ml), washed with water (x2) and brine. The solvent was evaporated to give methyl [6-(5-{3-cyano-4-[(1-methylethyl)oxy]phenyl}-1,2,4-oxadiazol-3-yl)-5-methyl-3,4-dihydro-2(1H)-isoquinolinyl]acetate (1.6 g) as a colourless solid.

LCMS (Method formate): Retention time 0.96 min, MH⁺=447

### Preparation 190

### 5-{5-[3-(2,2-dimethyl-1,3-dioxan-5-yl)-2,3,4,5-tetrahydro-1H-3-benzazepin-7-yl]-1,3,4-thiadiazol-2-yl}-2-[(1-methylethyl)oxy]benzonitrile

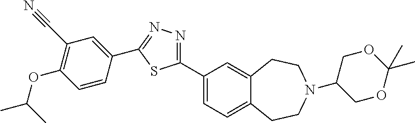

To a suspension of 2-[(1-methylethyl)oxy]-5-[3-(2,3,4,5-tetrahydro-1H-3-benzazepin-7-yl)-1,3,4-thiadiazol-2-yl]benzonitrile trifluoroacetate (Preparation 47) (160 mg, 0.32 mmol) and 2,2-dimethyl-1,3-dioxan-5-one (0.11 ml, 0.95 mmol) in DCM (5 ml) was added sodium triacetoxyborohydride (336 mg, 1.6 mmol) portionwise. The resulting mixture was stirred at room temperature for 72 h. A saturated aqueous solution of sodium hydrogen carbonate was added and the mixture was stirred for 15 min. The phases were separated, and the aqueous layer was extracted with DCM (x3). The combined organic layers were washed with brine, dried (MgSO₄), filtered and concentrated in vacuo to give 5-{5-[3-(2,2-dimethyl-1,3-dioxan-5-yl)-2,3,4,5-tetrahydro-1H-3-benzazepin-7-yl]-1,3,4-thiadiazol-2-yl}-2-[(1-methylethyl)oxy]benzonitrile (249 mg) as a viscous yellow oil which was used without further purification on the subsequent reaction (Example 199).

LCMS (Method formate): Retention time 0.94 min, MH⁺=505

### Preparation 191

### 5-{5-[3-(Bromoacetyl)-2,3,4,5-tetrahydro-1H-3-benzazepin-7-yl]-1,3,4-thiadiazol-2-yl}-2-[(1-methylethyl)oxy]benzonitrile

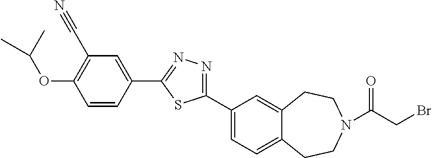

2-[(1-methylethyl)oxy]-5-[3-(2,3,4,5-tetrahydro-1H-3-benzazepin-7-yl)-1,3,4-thiadiazol-2-yl]benzonitrile trifluoroacetate (Preparation 47) (250 mg, 0.50 mmol) and DIPEA (0.22 ml, 1.2 mmol) were dissolved in DCM (2.5 ml) and the reaction treated with bromoacetyl bromide (0.043 ml, 0.50 mmol) as a solution in DCM (1 ml) added dropwise under nitrogen. The reaction mixture was stirred at room temperature for 1.5 h. The solvent was evaporated and the residue redissolved in ethyl acetate (10 ml). The solution was washed with water (20 ml) and brine (20 ml). The organic phase was dried and evaporated. The residue was purified by by chromatography on a silica cartridge (25 g), eluting with a methanol/DCM gradient (0-5%), to give 5-{5-[3-(bromoacetyl)-2,3,4,5-tetrahydro-1H-3-benzazepin-7-yl]-1,3,4-thiadiazol-2-yl}-2-[(1-methylethyl)oxy]benzonitrile as a brown solid (128 mg, 50%).

LCMS (Method formate): Retention time 1.21 min, MH⁺=511/513

### Preparation 192

### 6-(5-{3-chloro-4-[(1-methylethyl)oxy]phenyl}-1,3,4-thiadiazol-2-yl)-5-methyl-1,2,3,4-tetrahydroisoquinoline trifluoroacetate

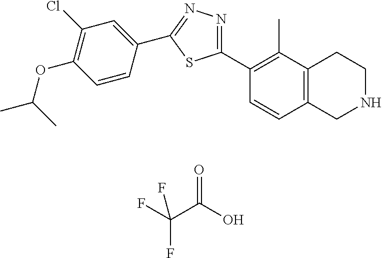

1,1-Dimethylethyl 6-(5-{3-chloro-4-[(1-methylethyl)oxy]phenyl}-1,3,4-thiadiazol-2-yl)-5-methyl-3,4-dihydro-2(1H)-isoquinolinecarboxylate (Preparation 193) (473 mg, 0.95 mmol) was dissolved in DCM (5 ml) and trifluoroacetic acid (2.5 ml, 0.95 mmol) was added to the solution. The reaction mixture was left standing for 30 min at 25° C. The solvent was removed by evaporation under reduced pressure and the crude product was redissolved in toluene and re-evaporated (x2). The residue was triturated with diethyl ether to give 6-(5-{3-chloro-4-[(1-methylethyl)oxy]phenyl}-1,3,4-thiadiazol-2-yl)-5-methyl-1,2,3,4-tetrahydroisoquinoline trifluoroacetate as a yellow solid (504 mg, 100%).

LCMS (Method formate): Retention time 0.94 min, MH⁺=400

### Preparation 193

### 1,1-Dimethylethyl 6-(5-{3-chloro-4-[(1-methylethyl)oxy]phenyl}-1,3,4-thiadiazol-2-yl)-5-methyl-3,4-dihydro-2(1H)-isoquinolinecarboxylate

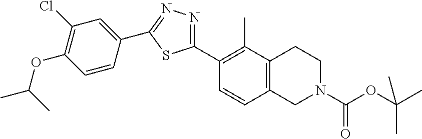

1,1-Dimethylethyl 5-methyl-6-(4,4,5,5-tetramethyl-1,3,2-dioxaborolan-2-yl)-3,4-dihydro-2(1H)-isoquinolinecarboxylate (Preparation 32) (2.06 g, 5.5 mmol) and 2-bromo-5-{3-chloro-4-[(1-methylethyl)oxy]phenyl}-1,3,4-thiadiazole (Preparation 2) (2.0 g, 6.0 mmol) were dissolved in mixture of 1,2-dimethoxyethane (30 ml) and water (10 ml) and stirred at room temperature under nitrogen atmosphere for 10 min. Sodium carbonate (1.76 g, 17 mmol) and bis(diphenylphosphino)ferrocenedichloro palladium (II) DCM complex (0.451 g, 0.55 mmol) were added to the reaction mixture, which was heated at 105° C. for 3 h and then overnight. The reaction mixture was cooled, the solvents evaporated under reduced pressure and the residue was partitioned between ethyl acetate (50 ml) and water (50 ml). The organic phase was washed with water (2×30 ml) and evaporated under reduced pressure. The crude product was redissolved in DCM and purified by chromatography on a silica cartridge (100 g), eluting with an ethyl acetate/cyclohexane gradient (0-100%). The fractions containing clean product were combined and the solvent was removed by evaporation. Fractions containing product contaminated with impurities were repurified by chromatography on a silica cartridge (25 g), eluting with an ethyl acetate/cyclohexane gradient (0-100%). Fractions containing clean product were combined with the previous clean product and the solvent was removed under reduced pressure. Fractions containing product contaminated with impurities were purified by MDAP (method formate) and then were added to the clean product. The solvent was removed by evaporation to yield 1,1-dimethylethyl 6-(5-{3-chloro-4-[(1-methylethyl)oxy]phenyl}-1,3,4-thiadiazol-2-yl)-5-methyl-3,4-dihydro-2(1H)-isoquinolinecarboxylate (473 mg, 17%).

LCMS (Method formate): Retention time 1.61 min, MH⁺=500/502

### Preparation 194

### 6-(5-{3-chloro-4-[(1-methylethyl)oxy]phenyl}-1,3,4-thiadiazol-2-yl)-2-(2,2-dimethyl-1,3-dioxan-5-yl)-5-methyl-1,2,3,4-tetrahydroisoquinoline

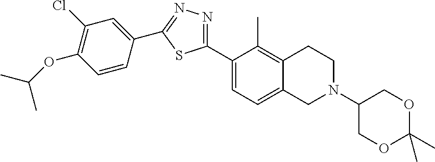

To a suspension of 6-(5-{3-chloro-4-[(1-methylethyl)oxy]phenyl}-1,3,4-thiadiazol-2-yl)-5-methyl-1,2,3,4-tetrahydroisoquinoline trifluoroacetate (Preparation 192) (135 mg, 0.26 mmol) and 2,2-dimethyl-1,3-dioxan-5-one (0.094 ml, 0.79 mmol) in DCM (4 ml) was added sodium triacetoxyborohydride (278 mg, 1.3 mmol) portionwise and the resulting mixture was stirred at room temperature for 2.5 h. A saturated aqueous solution of sodium hydrogen carbonate was added and the mixture was stirred for 15 min. The phases were separated, and the aqueous layer was extracted (x3) with DCM. The combined organic layers were washed with brine, dried (MgSO₄), filtered and concentrated in vacuo to give 6-(5-{3-chloro-4-[(1-methylethyl)oxy]phenyl}-1,3,4-thiadiazol-2-yl)-2-(2,2-dimethyl-1,3-dioxan-5-yl)-5-methyl-1,2,3,4-tetrahydroisoquinoline (121 mg) as a viscous yellow oil.

LCMS (Method formate): Retention time 1.02 min, MH⁺=514/516

### Preparation 195

### 1,3-dibromo-2-ethylbenzene

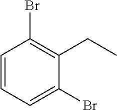

A three necked round-bottom flask was purged with argon and then filled with dry THF (60 mL), 1,3-dibromo benzene (9.5 g, 40.27 mmol) and ethyl iodide (8.8 g, 56.38 mmol). The mixture was cooled to −78° C. and LDA [made from 8 mL of iPr₂NH and 10 mL of BuLi (2.5M in hexane) in 40 mL of THF] was added slowly at −70° C. After stirring for 2 hr the reaction was poured into 100 mL of sat. aq. NH₄Cl solution and stirred vigorously for 20 min, extracted with DCM (2*100 mL). The organic layer was evaporated to give the crude product, which was purified by column chromatography with hexane to give 1,3-Dibromo-2-ethylbenzene (8.5 g, 79.9%).

6H(CDCl₃, 400 MHz): 7.76-7.78 (2H, m), 6.86-6.90 (1H, m), 2.89-3.01 (2H, q), 1.12 (3H, t) ppm. MS (ES⁺): C₈H₈Br₂ requires 264; found; 265 (M+H⁺).

### Preparation 196

### 3-bromo-2-ethyl-benzaldehyde

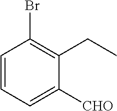

To a solution of 1,3-dibromo-2-ethyl-benzene (24 g, 90.92 mmol) (Preparation 195) in THF (300 mL) (D195) was added 36.4 mL of BuLi (2.5 M in hexane, 90.92 mmol) under N₂. The mixture was stirred for 2 hr at −78° C. Then DMF (12 g, 163.66 mmol) was added, after stirring for 2 hr, the reaction was poured onto 300 mL of sat. aq. NH₄Cl solution and extracted with DCM (2*100 mL). The organic layer was evaporated to give the crude product, which was purified by column chromatography eluting with EtOAc:hexane (1:20) to give 3-Bromo-2-ethylbenzaldehyde (7.6 g, yield 39.22%).

δH(CDCl₃, 400 MHz): 10.25 (1H, s), 7.76-7.78 (2H, m), 7.10-7.22 (1H, m), 3.20-3.27 (2H, q), 1.20 (3H, q) ppm. MS (ES⁺): C₉H₉BrO requires 213; found; 214 (M+H⁺).

### Preparation 197

### 1-bromo-2-ethyl-3-(2-nitrovinyl)benzene

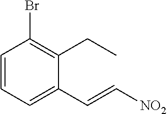

A mixture of 3-bromo-2-ethylbenzaldehyde (65.0 g, 307 mmol) (Preparation 196) and NH₄OAc (12.0 g, 154 mmol) in 200 mL of CH₃NO₂ was refluxed for 3 hours. The solvent was concentrated and purified on silica gel to give the desired product (66.9 g, 88.5% yield).

δH(CDCl₃, 400 MHz): 8.20 (1H, d), 7.59 (1H, d), 7.36-7.40 (1H, m), 7.02-7.06 (1H, m), 2.87-2.93 (4H, q), 1.12 (3H, t) ppm. MS (ES⁺): C₁₀H₁₀BrNO₂ requires 256; found; 257 (M+H⁺).

### Preparation 198

### 2-(3-bromo-2-ethyl phenyl)ethylamine

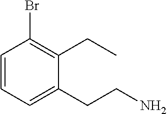

To a mixture of LiBH₄ (11.0 g, 500 mmol) and 300 mL of THF was added TMSCl (108 g, 1 mol) at 0° C. The reaction was stirred at 0° C. for 10 min, then added a solution of 1-bromo-2-ethyl-3-(2-nitrovinyl)benzene (25.5 g, 100 mmol) (Preparation 197) in 100 mL of THF. The reaction mixture was warmed to room temperature and refluxed for 3 hours. After cooling to room temperature, 40 mL of MeOH was added carefully. The solvent was concentrated, and diluted with 300 mL of EtOAc and 100 mL of water. The organic layer was dried over Na₂SO₄ and concentrated to give the crude product (19.0 g, 84.0% yield).

δH(CDCl₃, 400 MHz): 8.30 (2H, brs), 7.33-7.36 (1H, m), 7.04-7.19 (1H, m), 6.85-6.89 (1H, m), 3.09-3.19 (4H, m), 2.72-2.78 (2H, m), 1.08 (3H, t) ppm. MS (ES⁺): C₁₀H₁₄BrN requires 227; found; 228 (M+H⁺).

### Preparation 199

### N-[2-(3-bromo-2-ethylphenyl)ethyl]-2,2,2-trifluoroacetamide

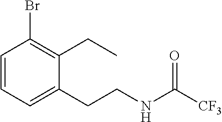

A mixture of 2-(3-bromo-2-ethylphenyl)ethylamine (19.0 g, 83.7 mmol) (Preparation 198), Et₃N (16.9 g, 167.4 mmol) and dry DCM (200 mL) was cooled to 0° C. Trifluoroacetic anhydride (35.2 g, 167.4 mmol) was added dropwise. The reaction was warmed to room temperature and allowed to stir for 16 hours. Water (100 mL) was added. The reaction was extracted with DCM (3×150 mL). The organic layer was dried over Na₂SO₄ and concentrated. Purification was performed on silica gel to give the desired product (25.79, 95.4% yield).

δH(CDCl₃, 400 MHz): 7.45 (2H, d), 6.90-7.10 (2H, m), 3.51-3.60 (2H, m), 2.64-2.95 (4H, m), 1.22 (3H, t) ppm. MS (ES⁺): C₁₂H₁₃BrF₃NO requires 323; found; 324 (M+H⁺).

### Preparation 200

### 1-(6-bromo-5-ethyl-3,4-dihydro-1H-isoquinolin-2-yl)-2,2,2-trifluoroethanone

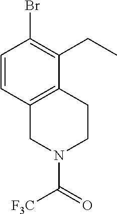

A mixture of glacial acetic (100 mL), concentrated sulfuric acid (40 mL), N-[2-(3-bromo-2-ethyl-phenyl)-ethyl]-2,2,2-trifluoro-acetamide (25.0 g, 77.4 mmol) (Preparation 199) and (CH₂O)ₙ (2.5 g) was heated at 70° C. for 4 hours, and then cooled to room temperature. The reaction mixture was poured into 200 mL of cold water, and extracted with EtOAc (3*200 mL). The organic layer was dried over Na₂SO₄ and concentrated. Purification was performed on silica gel to give the desired product.

6H(CDCl₃, 400 MHz): 7.41 (2H, d), 6.81 (2H, d), 4.66 (2H, d), 3.83-3.86 (2H, m), 2.94-2.98 (2H, m), 2.77-2.88 (2H, m), 1.19 (3H, t) ppm. MS (ES⁺): C₁₃H₁₃BrF₃NO requires 335; found; 336 (M+H⁺).

### Preparation 201

### 1-[5-ethyl-6-(4,4,5,5-tetramethyl-[1,3,2]dioxaborolan-2-yl)-3,4-dihydro-1H-isoquinolin-2-yl]-2,2,2-trifluoroethanone

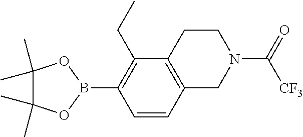

A mixture of 1-(6-bromo-5-ethyl-3,4-dihydro-1H-isoquinolin-2-yl)-2,2,2-trifluoroethanone (6.0 g, 17.9 mmol) (Preparation 200), 4,4,5,5,4′,4′,5′,5′-octamethyl-[2,2′]bi[[1,3,2]dioxaborolanyl] (5.46 g, 21.5 mmol), Pd(dppf)Cl₂ (1.31 g, 1.79 mmol) and CH₃COOK (3.5 g, 35.8 mmol) and 150 mL of 1,4-dioxane was refluxed for 10 hours. The reaction mixture was concentrated and purified on silica gel to give the desired product.

6H(CDCl₃, 400 MHz): 7.57 (2H, d), 6.91 (2H, d), 4.66 (2H, d), 3.79-3.82 (2H, m), 2.91-2.95 (4H, m), 1.25 (12H, s), 1.09 (3H, t) ppm. MS (ES⁺): C₁₉H₂₅BF₃NO₃ requires 383; found; 384 (M+H⁺).

### Preparation 202

### 2-hydroxy-5-iodobenzonitrile

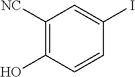

To a solution of 2-hydroxybenzonitrile (20 g, 0.168 mol) in CH₃CN (200 mL) was added dropwise CF₃SO₃H (16.5 mL) at 0° C. and followed by addition of NIS (45.4 g, 0.201 mol) at 0° C. The solution was stirred at rt overnight. The reaction mixtue was concentrated, washed with H₂O (1 L), extracted with DCM (800 mL*3). The combined organic layer was dried and concentrated. Purification was performed by column chromatography (PE:EA=10:1) to get 30 g of 2-hydroxy-5-iodo-benzonitrile (yield 73.2%).

δH (DMSO, 400 MHz): 7.68 (1H, s), 7.60-7.58 (1H, d), 6.67-6.65 (1H, d) ppm. MS (ES⁺): C₇H₄₁NO requires 245; found; 246 (M+H⁺).

### Preparation 203

### 5-iodo-2-isopropoxybenzonitrile

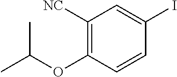

A mixture of 2-hydroxy-5-iodobenzonitrile (42 g, 0.173 mol) (Preparation 202), iPrl (58.7 g, 0.345 mol) and K₂CO₃ (47.7 g 0.345 mol) and CH₃CN (420 mL) was stirred at reflux overnight. The reaction mixture was filtered and concentrated. The residue was diluted with DCM (300 mL) and filtered. The filtrate was concentrated to get 48.5 g of 5-iodo-2-isopropoxybenzonitrile (yield 97.6%)

δH(CDCl₃, 400 MHz): 7.79 (1H, s), 7.75-7.73 (1H, d), 6.74-6.72 (1H, d), 4.62-4.59 (1H, m), 1.39-1.37 (6H, s) ppm. MS (ES⁺): C₁₀H₁₀INO requires 287; found; 288 (M+H⁺).

### Preparation 204

### 2-isopropoxy-5-(4,4,5,5-tetramethyl-[1,3,2]dioxaborolan-2-yl)benzonitrile

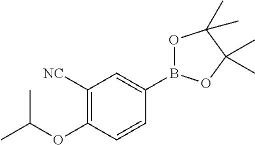

A mixture of 5-iodo-2-isopropoxybenzonitrile (15 g, 0.052 mol) (Preparation 203), Pin₂B₂ (15.9 g, 0.0627 mol), Pd(dppf)Cl₂ (4.3 g, 5.2 mmol) and KOAc (15.3 g, 0.156 mol) and dioxane (150 mL) was stirred at 90° C. under N₂ overnight. The reaction mixture was filtered and concentrated. Purification was performed by column chromatography (PE:EA=200:1) to get 13.8 g of 2-isopropoxy-5-(4,4,5,5-tetramethyl-[1,3,2]dioxaborolan-2-yl)-benzonitrile(yield 92%).

δH(CDCl₃, 400 MHz): 7.9-8.0 (1H, d), 7.85-7.89 (1H, dd), 6.92-6.90 (1H, d), 4.65-4.74 (1H, m), 1.39-1.37 (6H, s), 1.24 (6H, s) ppm. MS (ES⁺): C₁₆H₂₂BNO₃ requires 287; found; 288 (M+H⁺).

### Preparation 205

### 2-isopropoxy-5-thiazol-2-ylbenzonitrile

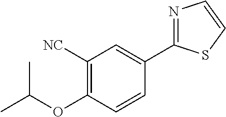

A mixture of 2-isopropoxy-5-(4,4,5,5-tetramethyl-[1,3,2]dioxaborolan-2-yl)benzonitrile (5 g, 17.4 mmol), 2-bromo-thiazole (3.43 g, 20.9 mmol) (Preparation 204), Pd(dppf)Cl₂ (1.4 g, 1.74 mmol) and Na₂CO₃(3.7 g, 34.8 mmol) and DME/H₂O(100 mL, 1:1) was stirred at reflux under N₂ overnight. The reaction mixture was cooled to rt, extracted with DCM(300 mL*3). The organic layer was dried and concentrated. Purification was performed by column chromatography (PE:EA=10:1) to get 2 g of 2-isopropoxy-5-thiazol-2-yl-benzonitrile(yield 47.1%).

δH(CDCl₃, 400 MHz): 8.13 (1H, s), 8.10-8.17 (1H, d), 7.83-7.82 (1H, d), 7.32-7.31 (1H, d), 7.03-7.00 (1H, d), 4.73-4.70 (1H, m), 1.43-1.41 (6H, s) ppm. MS (ES⁺): C₁₃H₁₂N₂OS requires 244; found; 245 (M+H⁺).

### Preparation 206

### 5-(5-bromo-thiazol-2-yl)-2-isopropoxybenzonitrile

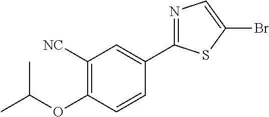

To a mixture of 2-isopropoxy-5-thiazol-2-ylbenzonitrile (1.88 g, 7.7 mmol) (Preparation 205) and DMF (19 mL) was added NBS (2.74 g, 15.4 mmol) at 0° C. After addition the resulting mixture was stirred at rt for 3 h. H₂O (100 mL) was added. The reaction mixture was extracted with DCM (100 mL*3), washed by water (100 mL*3). The organic phase was separated, dried and concentrated. Purification was performed by column chromatography (PE:EA=20:1) to get 1.9 g of 5-(5-bromo-thiazol-2-yl)-2-isopropoxy-benzonitrile (yield 76.3%). MS (ES⁺): C₁₃H₁₁BrN₂OS requires 322; found; 323 (M+H⁺).

### Preparation 207

### 5-[5-(5-ethyl-3,4-dihydro-isoquinolin-6-yl)-thiazol-2-yl]-2-isopropoxybenzonitrile

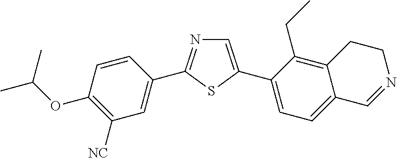

A mixture of 5-(5-bromo-thiazol-2-yl)-2-isopropoxybenzonitrile (400 mg, 1.24 mmol) (Preparation 206), 1-[5-ethyl-6-(4,4,5,5-tetramethyl-[1,3,2]dioxaborolan-2-yl)-3,4-dihydro-1H-isoquinolin-2-yl]-2,2,2-trifluoroethanone (570 mg, 1.49 mmol) (Preparation 201), Pd(dppf)Cl₂ (101 mg, 0.124 mmol) and Na₂CO₃ (262 mg, 2.48 mmol) and DME/H₂O (8 mL, 1:1) was stirred at reflux under N₂ overnight. The reaction mixture was cooled to rt, extracted with DCM (20 mL*3). The combined organic layer was dried and concentrated. Purification was performed by prep-HPLC to get 260 mg of 5-[5-(5-ethyl-3,4-dihydro-isoquinolin-6-yl)-thiazol-2-yl]-2-isopropoxybenzonitrile (yield 52.2%).

δH(CDCl₃, 400 MHz): 8.15-8.10 (2H, m), 7.82-7.70 (1H, d), 7.55-7.54 (1H, m), 7.11-7.08 (2H, d), 7.28-7.26 (1H, d), 7.08-7.01 (2H, m), 4.76-4.72 (1H, m), 4.40 (1H, s), 4.10-4.08 (1H, d), 3.54 (1H, 1), 3.25-3.23 (2H, d), 2.85-2.84 (1H, m), 2.71-2.69 (1H, m), 1.46-1.43 (6H, m), 1.21-1.10 (3H, m) ppm. MS (ES⁺): C₂₄H₂₃N₃OS requires 401; found; 402 (M+H⁺).

### Preparation 208

### 5-[5-(5-ethyl-1,2,3,4-tetrahydro-isoquinolin-6-yl)-thiazol-2-yl]-2-isopropoxybenzonitrile

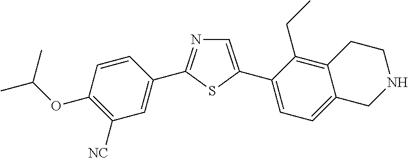

A solution of 5-[5-(5-ethyl-3,4-dihydro-isoquinolin-6-yl)-thiazol-2-yl]-2-isopropoxy-benzo nitrile (250 mg, 0.62 mmol) (Preparation 207) and NaBH₄ (70 mg, 1.24 mmol) in EtOH (5 mL) was stirred at reflux for 2 h. The reaction mixture was concentrated, diluted with DCM (30 mL), filtered, dried over Na₂SO₄ and concentrated. Purification was performed by prep-HPLC to get 100 mg of 5-[5-(5-Ethyl-1,2,3,4-tetrahydro-isoquinolin-6-yl)-thiazol-2-yl]-2-isopropoxy-benzo nitrile (yield 40%).

δH(CDCl₃, 400 MHz): 8.12 (1H, s), 8.10-8.09 (1H, s), 7.70 (1H, s), 7.29-7.25 (1H, d), 7.28-7.26 (1H, d), 7.08-7.02 (2H, m), 4.77-4.71 (1H, m), 4.39 (2H, m), 3.60-3.48 (2H, m), 3.40-3.06 (6H, m), 2.75-2.68 (2H, m), 1.51-1.44 (6H, s), 1.14-1.10 (3H, m) ppm. MS (ES⁺): C₂₄H₂₅N₃OS requires 403; found; 404 (M+H⁺).

### Preparation 209

### 5-(5-bromo-[1,3,4]thiadiazol-2-yl)-2-isopropoxybenzonitrile

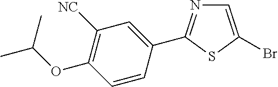

A mixture of 2-isopropoxy-5-(4,4,5,5-tetramethyl-[1,3,2]dioxaborolan-2-yl)benzonitrile (1 g, 3.48 mmol) (Preparation 204), 2,5-dibromo-[1,3,4]thiadiazole (1.02 g, 4.18 mmol), Pd(Ph₃)₄ (400 mg, 0.348 mmol) and Na₂CO₃ (740 mg, 6.96 mmol) in DME/H₂O (20 mL, 1:1) was stirred at reflux under N₂ overnight. The reaction mixture was cooled to rt, extracted with DCM (60 mL*3). The combined organic layer was dried and concentrated. Purification was performed by column chromatography (PE:EA=10:1) to get 400 mg of 2-bromo-5-(3-isocyano-4-isopropoxy-phenyl)-[1,3,4]thiadiazole(yield 47.1%).

MS (ES⁺): C₁₂H₁₀BrN₃OS requires 323; found; 324 (M+H⁺).

### Preparation 210

### 5-ethyl-6-[5-(3-cyano-4-isopropoxy-phenyl)-[1,3,4]thiadiazol-2-yl]-1,2,3,4-tetrahydroisoquinoline

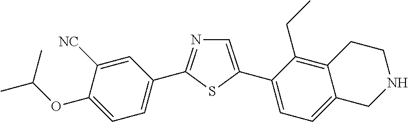

A mixture of 5-(5-bromo-[1,3,4]thiadiazol-2-yl)-2-isopropoxy-benzonitrile (400 mg, 1.07 mmol) (Preparation 209), 1-[5-ethyl-6-(4,4,5,5-tetramethyl-[1,3,2]dioxaborolan-2-yl)-3,4-dihydro-1H-isoquinolin-2-yl]-2,2,2-trifluoro-ethanone (490 mg, 1.28 mmol) (Preparation 201), Pd(dppf)Cl₂ (87 mg, 0.107 mmol), Na₂CO₃ (230 mg, 2.14 mmol) and DME/H₂O (8 mL, 1:1) was stirred at reflux under N₂ overnight. The reaction mixture was cooled to rt, extracted with DCM (30 mL*3). The combined organic layer was dried and concentrated. Purification was performed by pre-HPLC to get 100 mg of 5-ethyl-6-[5-(3-cyano-4-isopropoxy-phenyl)-[1,3,4]thiadiazol-2-yl]-1,2,3,4-tetrahydroisoquinoline.

6H(CDCl₃, 400 MHz): 8.22-8.19 (1H, m), 8.16 (1H, s), 7.82-7.46 (1H, d), 7.11-7.08 (2H, d), 4.79-4.73 (1H, m), 4.49-4.40 (2H, m), 3.65-3.53 (2H, m), 3.22 (2H, s), 2.90-2.85 (2H, m), 1.47-1.45 (6H, d), 1.24-1.19 (3H, m) ppm. MS (ES⁺): C₂₃H₂₄N₄OS requires 404; found; 405 (M+H⁺).

### Example 1

### 3-[6-(5-{3-Cyano-4-[(1-methylethyl)oxy]phenyl}-1,2,4-oxadiazol-3-yl)-5-methyl-3,4-dihydro-2(1H)-isoquinolinyl]propanamide

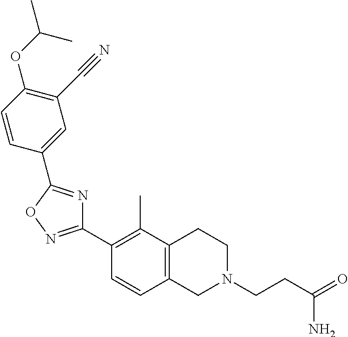

A mixture of 3-[6-(5-{3-cyano-4-[(1-methylethyl)oxy]phenyl}-1,2,4-oxadiazol-3-yl)-5-methyl-3,4-dihydro-2(1H)-isoquinolinyl]propanoic acid sodium salt (Preparation 27) (60 mg), triethylamine (37 μl, 0.27 mmol), EDC (31 mg, 0.16 mmol) and HOBT ammonium salt (31 mg, 0.20 mmol) in DMF (2 mlml) was stirred at room temperature for 24 h. The reaction mixture was diluted with saturated sodium hydrogen carbonate (5 ml) and extracted with ethyl acetate (3×5 ml). The combined organic phases were washed with brine, dried and concentrated in vacuo. Purification of the residue chromatography (methanol/DCM 0-5%) gave 3-[6-(5-{3-cyano-4-[(1-methylethyl)oxy]phenyl}-1,2,4-oxadiazol-3-yl)-5-methyl-3,4-dihydro-2(1H)-isoquinolinyl]propanamide (15 mg) as a colourless solid.

LCMS (Method formate): Retention time 0.88 min, MH⁺=446

### Example 2

### 3-[6-(5-{3-Cyano-4-[(1-methylethyl)oxy]phenyl}-1,2,4-oxadiazol-3-yl)-5-methyl-3,4-dihydro-2(1H)-isoquinolinyl]-N,N-dimethylpropanamide

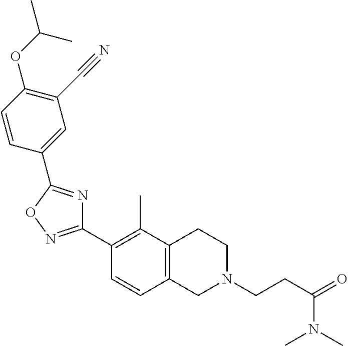

A mixture of 3-[6-(5-{3-cyano-4-[(1-methylethyl)oxy]phenyl}-1,2,4-oxadiazol-3-yl)-5-methyl-3,4-dihydro-2(1H)-isoquinolinyl]propanoic acid sodium salt (Preparation 27) (60 mg), triethylamine (37 μl, 0.27 mmol), EDC (31 mg, 0.16 mmol), 1-hydroxybenzotriazole hydrate (25 mg, 0.16 mmol), and dimethylamine solution in THF (2M, 130 μl, 0.26 mmol) in DMF (2 ml) was stirred at room temperature for 24 h. The reaction mixture was diluted with saturated sodium hydrogen carbonate (5 ml) and extracted with ethyl acetate (3×5 ml). The combined extracts were washed with brine, dried and concentrated. Purification of the residue by chromatography (methanol/DCM, 0-5%) gave 3-[6-(5-{3-cyano-4-[(1-methylethyl)oxy]phenyl}-1,2,4-oxadiazol-3-yl)-5-methyl-3,4-dihydro-2(1H)-isoquinolinyl]-N,N-dimethylpropanamide (40 mg) as a colourless solid.

LCMS (Method formate): Retention time 0.92 min, MH⁺=474

### Example 3

### 2-[6-(5-{3-Cyano-4-[(1-methylethyl)oxy]phenyl}-1,2,4-oxadiazol-3-yl)-5-methyl-3,4-dihydro-2(1H)-isoquinolinyl]acetamide

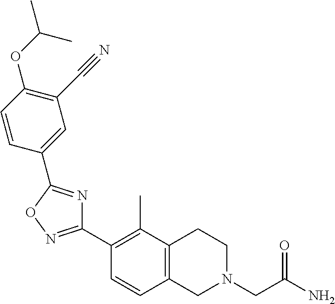

A mixture of [6-(5-{3-cyano-4-[(1-methylethyl)oxy]phenyl}-1,2,4-oxadiazol-3-yl)-5-methyl-3,4-dihydro-2(1H)-isoquinolinyl]acetic acid sodium salt (Preparation 29) (100 mg), triethylamine (64 μl, 0.46 mmol), EDC (53 mg, 0.28 mmol) and HOBT ammonium salt (53 mg, 0.35 mmol) in DCM (3 ml) was stirred at room temperature for overnight then concentrated. The residue was dissolved in ethyl acetate (10 ml) and the organic phase was washed sequentially with saturated sodium hydrogen carbonate, water and brine, dried and concentrated. Purification of the residue by chromatography (methanol/DCM, 5-10%) gave 2-[6-(5-{3-cyano-4-[(1-methylethyl)oxy]phenyl}-1,2,4-oxadiazol-3-yl)-5-methyl-3,4-dihydro-2(1H)-isoquinolinyl]acetamide (75 mg) as a colourless solid.

LCMS (Method formate): Retention time 0.90 min, MH⁺=432

### Example 4

### 4-[6-(5-{3-Cyano-4-[(1-methylethyl)oxy]phenyl}-1,2,4-oxadiazol-3-yl)-5-methyl-3,4-dihydro-2(1H)-isoquinolinyl]butanamide

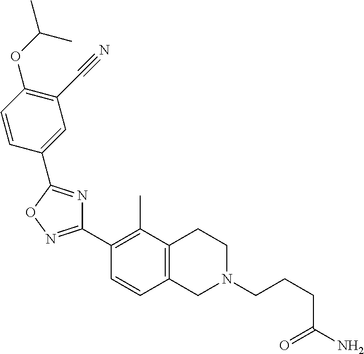

A mixture of 4-[6-(5-{3-cyano-4-[(1-methylethyl)oxy]phenyl}-1,2,4-oxadiazol-3-yl)-5-methyl-3,4-dihydro-2(1H)-isoquinolinyl]butanoic acid sodium salt (Preparation 31) (60 mg), triethylamine (36 μl, 0.26 mmol), EDC (30 mg, 0.16 mmol), and HOBT ammonium salt (30 mg, 0.19 mmol) in DMF (2 ml) was stirred at room temperature for 24 h. The reaction mixture was diluted with saturated sodium hydrogen carbonate (5 ml) and extracted with ethyl acetate (3×5 ml). The combined extracts were washed with brine, dried and evaporated. Purification of the residue by chromatography (methanol/DCM 0-5%) gave 4-[6-(5-{3-cyano-4-[(1-methylethyl)oxy]phenyl}-1,2,4-oxadiazol-3-yl)-5-methyl-3,4-dihydro-2(1H)-isoquinolinyl]butanamide (22 mg) as a colourless solid.

LCMS (Method formate): Retention time 0.89 min, MH⁺=460

### Example 5

### 4-[6-(5-{3-Cyano-4-[(1-methylethyl)oxy]phenyl}-1,2,4-oxadiazol-3-yl)-5-methyl-3,4-dihydro-2(1H)-isoquinolinyl]-N-methylbutanamide

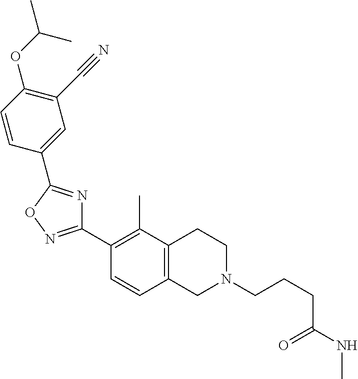

A mixture of 4-[6-(5-{3-cyano-4-[(1-methylethyl)oxy]phenyl}-1,2,4-oxadiazol-3-yl)-5-methyl-3,4-dihydro-2(1H)-isoquinolinyl]butanoic acid sodium salt (Preparation 31) (60 mg), triethylamine (36 μl, 0.26 mmol), EDC (30 mg, 0.16 mmol), HOBT (24 mg, 0.16 mmol) and methylamine solution in THF (2M, 130 μl, 0.26 mmol) in DMF (2 ml) was stirred at room temperature for 24 h. The reaction mixture was diluted with saturated sodium hydrogen carbonate (5 ml) and extracted with ethyl acetate (3×5 ml). The combined extracts were washed with brine, dried and concentrated. Purification of the residue by chromatography (methanol/DCM 0-5%) gave 4-[6-(5-{3-cyano-4-[(1-methylethyl)oxy]phenyl}-1,2,4-oxadiazol-3-yl)-5-methyl-3,4-dihydro-2(1H)-isoquinolinyl]-N-methylbutanamide (14 mg) as a colourless solid.

LCMS (Method formate): Retention time 0.92 min, MH⁺=474

### Example 6

### 5-{3-[2-(2,3-Dihydroxypropyl)-5-methyl-1,2,3,4-tetrahydro-6-isoquinolinyl]-1,2,4-oxadiazol-5-yl}-2-[(1-methylethyl)oxy]benzonitrile

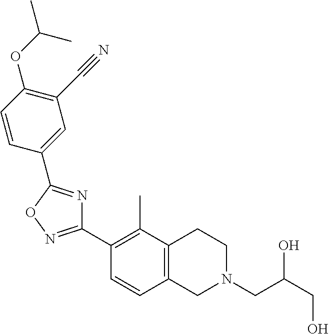

Glacial acetic acid (12 μl, 0.28 mmol) was added to a solution of 2-[(1-methylethyl)oxy]-5-[3-(5-methyl-1,2,3,4-tetrahydro-6-isoquinolinyl)-1,2,4-oxadiazol-5-yl]benzonitrile trifluoroacetic acid salt (Preparation 25) (100 mg, 0.2 mmol) in THF (3 ml) and 1,2-dichloroethane (3 ml) followed by DL-glyceraldehyde (92 mg, 1.0 mmol). The mixture was stirred at room temperature for 1 h, and sodium triacetoxyborohydride (220 mg, 1.0 mmol) added. The reaction was stirred overnight and a further portion of sodium triacetoxyborohydride (100 mg) added. The reaction was stirred for 24 h a further portion of sodium triacetoxyborohydride (100 mg) added to the mixture. Saturated sodium hydrogen carbonate aqueous solution (10 ml) was added 6 h after the last addition of sodium triacetoxyborohydride and the resulting mixture extracted with ethyl acetate (3×10 ml). The combined extracts were dried and concentrated. Purification of the residue by chromatography (methanol/DCM, 15%) gave 5-{3-[2-(2,3-dihydroxypropyl)-5-methyl-1,2,3,4-tetrahydro-6-isoquinolinyl]-1,2,4-oxadiazol-5-yl}-2-[(1-methylethyl)oxy]benzonitrile (60 mg, 65%) as a pale yellow solid.

LCMS (Method formate): Retention time 0.92 min, MH+ not seen.

### Example 7

### 5-{3-[2-(2-Hydroxyethyl)-5-methyl-1,2,3,4-tetrahydro-6-isoquinolinyl]-1,2,4-oxadiazol-5-yl}-2-[(1-methylethyl)oxy]benzonitrile

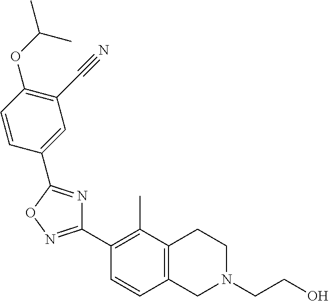

Potassium carbonate (37 mg, 0.27 mmol) was added to stirred solution of 2-[(1-methylethyl)oxy]-5-[3-(5-methyl-1,2,3,4-tetrahydro-6-isoquinolinyl)-1,2,4-oxadiazol-5-yl]benzonitrile trifluoroacetic acid salt (Preparation 25) (60 mg, 0.12 mmol) and 2-bromoethanol (10 μl, 0.13 mmol) in dry DMF (2 ml). The reaction mixture was stirred at 80° C. for 6 h and diluted with ethyl acetate (10 ml). The organic phase was washed with water (x2), then brine, dried and concentrated. Purification of the residue by chromatography (methanol/DCM, 0-5%) gave 5-{3-[2-(2-hydroxyethyl)-5-methyl-1,2,3,4-tetrahydro-6-isoquinolinyl]-1,2,4-oxadiazol-5-yl}-2-[(1-methylethyl)oxy]benzonitrile (33 mg, 64%) as a colourless foam which solidified on trituration with diethyl ether and hexane.

LCMS (Method formate): Retention time 0.91 min, MH⁺=419

### Example 8

### 5-{3-[2-(3-Hydroxypropyl)-5-methyl-1,2,3,4-tetrahydro-6-isoquinolinyl]-1,2,4-oxadiazol-5-yl}-2-[(1-methylethyl)oxy]benzonitrile

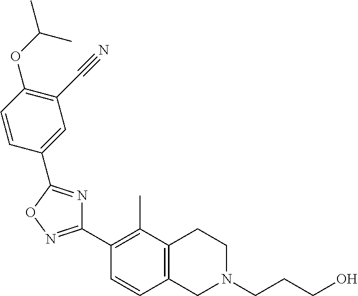

Potassium carbonate (37 mg, 0.27 mmol) was added to stirred solution of 2-[(1-methylethyl)oxy]-5-[3-(5-methyl-1,2,3,4-tetrahydro-6-isoquinolinyl)-1,2,4-oxadiazol-5-yl]benzonitrile trifluoroacetic acid salt (Preparation 25) (60 mg, 0.12 mmol) and 3-bromopropan-1-ol (12 μl, 0.13 mmol) in dry DMF (2 ml). The reaction mixture was stirred at 80° C. for 6 h and diluted with ethyl acetate (10 ml). The organic phase was washed with water (x2), then brine, dried and concentrated. Purification of the residue by chromatography (methanol/DCM, 0-5%) gave 5-{3-[2-(3-hydroxypropyl)-5-methyl-1,2,3,4-tetrahydro-6-isoquinolinyl]-1,2,4-oxadiazol-5-yl}-2-[(1-methylethyl)oxy]benzonitrile (48 mg, 90%) as a colourless oil which solidified on trituration with diethyl ether/hexane.

LCMS (Method formate): Retention time 0.92 min, MH⁺=433

### Example 9

### 2-[(1-Methylethyl)oxy]-5-(3-{5-methyl-2-[2-(methyloxy)ethyl]-1,2,3,4-tetrahydro-6-isoquinolinyl}-1,2,4-oxadiazol-5-yl)benzonitrile

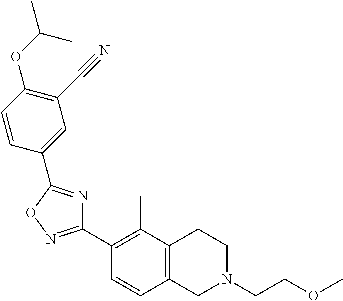

Potassium carbonate (37 mg, 0.27 mmol) was added to stirred solution of 2-[(1-methylethyl)oxy]-5-[3-(5-methyl-1,2,3,4-tetrahydro-6-isoquinolinyl)-1,2,4-oxadiazol-5-yl]benzonitrile trifluoroacetic acid salt (Preparation 25) (60 mg, 0.12 mmol) and 2-bromoethyl methyl diethyl ether (13 μl, 0.13 mmol) in dry DMF (2 ml). The reaction mixture was stirred at 80° C. for 6 h and diluted with ethyl acetate (10 ml). The organic phase was washed with water (x2), then brine, dried and concentrated. Purification of the residue by chromatography (methanol/DCM, 0-5%) gave 2-[(1-methylethyl)oxy]-5-(3-{5-methyl-2-[2-(methyloxy)ethyl]-1,2,3,4-tetrahydro-6-isoquinolinyl}-1,2,4-oxadiazol-5-yl)benzonitrile (38 mg, 71%) as a colourless oil which solidified on trituration with diethyl ether and hexane.

LCMS (Method formate): Retention time 0.97 min, MH⁺=433

### Example 10

### 3-[6-(5-{3-Cyano-4-[(1-methylethyl)oxy]phenyl}-1,3,4-thiadiazol-2-yl)-5-methyl-3,4-dihydro-2(1H)-isoquinolinyl]propanamide

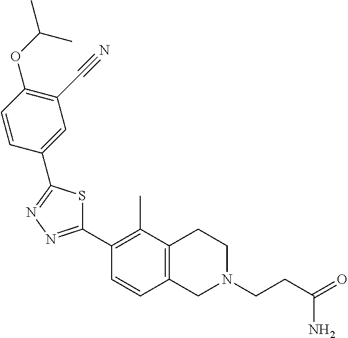

A mixture of 3-[6-(5-{3-cyano-4-[(1-methylethyl)oxy]phenyl}-1,3,4-thiadiazol-2-yl)-5-methyl-3,4-dihydro-2(1H)-isoquinolinyl]propanoic acid sodium salt (Preparation 36) (130 mg), triethylamine (78 μl, 0.56 mmol), EDC (65 mg, 0.34 mmol) and HOBT ammonium salt (65 mg, 0.42 mmol) in DMF (3 ml) was stirred at room temperature overnight then at 40° C. for 8 h. The reaction mixture was cooled and diluted with ethyl acetate (5 ml). The mixture was washed sequentially with saturated sodium hydrogen carbonate, water and brine, the organic phase dried and concentrated. Purification of the residue by chromatography (methanol/DCM, 0-5%) gave 3-[6-(5-{3-cyano-4-[(1-methylethyl)oxy]phenyl}-1,3,4-thiadiazol-2-yl)-5-methyl-3,4-dihydro-2(1H)-isoquinolinyl]propanamide (62 mg) as an off-white solid.

LCMS (Method formate): Retention time 0.85 min, MH⁺=462

### Example 11

### 3-[6-(5-{3-Cyano-4-[(1-methylethyl)oxy]phenyl}-1,3,4-thiadiazol-2-yl)-5-methyl-3,4-dihydro-2(1H)-isoquinolinyl]-N-methylpropanamide

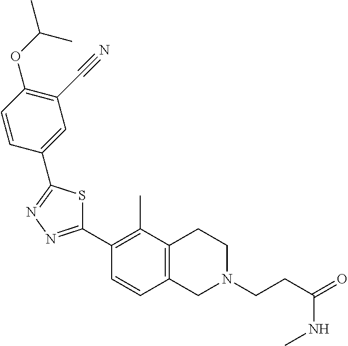

A mixture of 3-[6-(5-{3-cyano-4-[(1-methylethyl)oxy]phenyl}-1,3,4-thiadiazol-2-yl)-5-methyl-3,4-dihydro-2(1H)-isoquinolinyl]propanoic acid sodium salt (Preparation 36) (130 mg), triethylamine (78 μl, 0.56 mmol), EDC (65 mg, 0.34 mmol), HOBT (52 mg, 0.34 mmol) and methylamine solution in THF (2M, 280 μl, 0.56 mmol) in DMF (3 ml) was stirred at room temperature overnight then at 40° C. for 8 h. The reaction mixture was then cooled to room temperature and diluted with ethyl acetate (5 ml). The mixture was washed sequentially with saturated sodium hydrogen carbonate, water and brine, the organic phase dried and concentrated. Purification of the residue by chromatography (methanol/DCM, 0-5%) gave 3-[6-(5-{3-cyano-4-[(1-methylethyl)oxy]phenyl}-1,3,4-thiadiazol-2-yl)-5-methyl-3,4-dihydro-2(1H)-isoquinolinyl]-N-methylpropanamide (30 mg) as a yellow oil which solidified.

LCMS (Method formate): Retention time 0.85 min, MH⁺=476

### Example 12

### 3-[6-(5-{3-Cyano-4-[(1-methylethyl)oxy]phenyl}-1,3,4-thiadiazol-2-yl)-5-methyl-3,4-dihydro-2(1H)-isoquinolinyl]-N,N-dimethylpropanamide

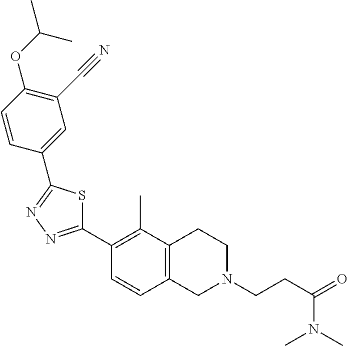

A mixture of 3-[6-(5-{3-cyano-4-[(1-methylethyl)oxy]phenyl}-1,3,4-thiadiazol-2-yl)-5-methyl-3,4-dihydro-2(1H)-isoquinolinyl]propanoic acid sodium salt (Preparation 36) (130 mg), triethylamine (78 μl, 0.56 mmol), EDC (65 mg, 0.34 mmol) HOBT (52 mg, 0.34 mmol) and dimethylamine solution in THF (2M, 280 μl, 0.56 mmol) in DMF (3 ml) was stirred at room temperature overnight then at 40° C. for 48 h. The reaction mixture was cooled and diluted with ethyl acetate (5 ml). The mixture was washed sequentially with saturated sodium hydrogen carbonate, water and brine, the organic phase dried and concentrated. Purification of the residue by chromatography (methanol/DCM, 0-5%) gave 3-[6-(5-{3-cyano-4-[(1-methylethyl)oxy]phenyl}-1,3,4-thiadiazol-2-yl)-5-methyl-3,4-dihydro-2(1H)-isoquinolinyl]-N,N-dimethylpropanamide (46 mg) as a yellow oil which solidified on standing.

LCMS (Method formate): Retention time 0.87 min, MH⁺=490

### Example 13

### 4-[6-(5-{3-Cyano-4-[(1-methylethyl)oxy]phenyl}-1,3,4-thiadiazol-2-yl)-5-methyl-3,4-dihydro-2(1H)-isoquinolinyl]butanamide

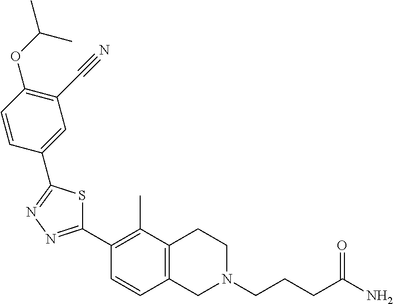

A mixture of 4-[6-(5-{3-cyano-4-[(1-methylethyl)oxy]phenyl}-1,3,4-thiadiazol-2-yl)-5-methyl-3,4-dihydro-2(1H)-isoquinolinyl]butanoic acid sodium salt (Preparation 38) (100 mg), triethylamine (58 μl, 0.42 mmol), EDC (48 mg, 0.25 mmol) and HOBT ammonium salt (48 mg, 0.32 mmol) in DCM (3 ml) was stirred at room temperature overnight then concentrated. The residue was dissolved in ethyl acetate (10 ml). The mixture was washed sequentially with saturated sodium hydrogen carbonate, water and brine, the organic phase dried and concentrated. Purification of the residue by chromatography (methanol/DCM, 0-5%) gave a light brown product which was further purified by MDAP (Method: formate) to give 4-[6-(5-{3-cyano-4-[(1-methylethyl)oxy]phenyl}-1,3,4-thiadiazol-2-yl)-5-methyl-3,4-dihydro-2(1H)-isoquinolinyl]butanamide (71 mg) as an off-white solid.

LCMS (Method formate): Retention time 0.86 min, MH⁺=476

### Example 14

### 4-[6-(5-{3-Cyano-4-[(1-methylethyl)oxy]phenyl}-1,3,4-thiadiazol-2-yl)-5-methyl-3,4-dihydro-2(1H)-isoquinolinyl]-N-methylbutanamide

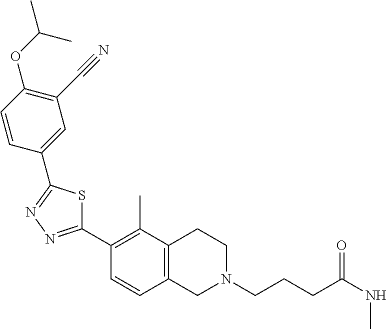

A solution of 4-[6-(5-{3-cyano-4-[(1-methylethyl)oxy]phenyl}-1,3,4-thiadiazol-2-yl)-5-methyl-3,4-dihydro-2(1H)-isoquinolinyl]butanoic acid sodium salt (Preparation 38) (90 mg, 0.18 mmol) was treated with glacial acetic acid (11 μl, 0.19 mmol) stirred for 5 min, cooled to 0° C. and treated with N-ethylmorpholine (50 μl, 0.4 mmol) followed by iso-butyl chloroformate (28 μl, 0.21 mmol). The mixture was stirred at 0° C. for 15 min. Aqueous methylamine (40% w/w, 1 ml) was added and the resulting mixture was stirred at room temperature for 1 h then at 120° C. for 1 h (microwave). The mixture was diluted with water and extracted with ethyl acetate (2×5 ml). The combined organic phases were dried and concentrated. Purification of the residue by MDAP (Method formate) gave 4-[6-(5-{3-cyano-4-[(1-methylethyl)oxy]phenyl}-1,3,4-thiadiazol-2-yl)-5-methyl-3,4-dihydro-2(1H)-isoquinolinyl]-N-methylbutanamide (18 mg, 20%) as a colourless solid.

LCMS (Method formate): Retention time 0.89 min, MH⁺=490.25

### Example 15

### 5-{5-[2-(2-Hydroxyethyl)-5-methyl-1,2,3,4-tetrahydro-6-isoquinolinyl]-1,3,4-thiadiazol-2-yl}-2-[(1-methylethyl)oxy]benzonitrile

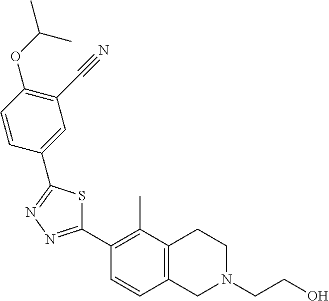

A mixture of 2-[(1-methylethyl)oxy]-5-[3-(5-methyl-1,2,3,4-tetrahydro-6-isoquinolinyl)-1,3,4-thiadiazol-2-yl]benzonitrile hydrochloride (Preparation 34) (100 mg, 0.23 mmol), 2-bromoethanol (18 μl, 0.26 mmol) and potassium carbonate (71 mg, 0.52 mmol) in dry DMF (3 ml) was stirred at 60° C. overnight, cooled to room temperature and diluted with ethyl acetate (10 ml). The mixture was washed with water (x2), then brine, dried and concentrated. Purification of the residue by chromatography (methanol/DCM, 0-5%) followed by trituration with diethyl ether gave 5-{5-[2-(2-hydroxyethyl)-5-methyl-1,2,3,4-tetrahydro-6-isoquinolinyl]-1,3,4-thiadiazol-2-yl}-2-[(1-methylethyl)oxy]benzonitrile (25 mg, 25%) as a colourless solid.

LCMS (Method formate): Retention time 0.88 min, MH⁺=435

### Example 16

### 5-{5-[2-(3-Hydroxypropyl)-5-methyl-1,2,3,4-tetrahydro-6-isoquinolinyl]-1,3,4-thiadiazol-2-yl}-2-[(1-methylethyl)oxy]benzonitrile

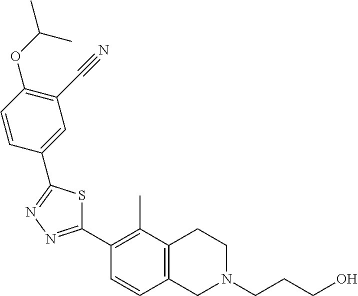

A mixture of 2-[(1-methylethyl)oxy]-5-[3-(5-methyl-1,2,3,4-tetrahydro-6-isoquinolinyl)-1,3,4-thiadiazol-2-yl]benzonitrile hydrochloride (Preparation 34) (100 mg, 0.23 mmol), 3-bromo-1-propanol (23 μl, 0.26 mmol) and potassium carbonate (71 mg, 0.52 mmol) in dry DMF (3 ml) was stirred at 60° C. overnight, cooled to room temperature and diluted with ethyl acetate (10 ml). The mixture was washed with water (x2), then brine, dried and concentrated. Purification of the residue by chromatography (methanol/DCM, 0-5%) followed by trituration with diethyl ether gave 5-{5-[2-(3-hydroxypropyl)-5-methyl-1,2,3,4-tetrahydro-6-isoquinolinyl]-1,3,4-thiadiazol-2-yl}-2-[(1-methylethyl)oxy]benzonitrile (46 mg, 44%) as a colourless solid.

LCMS (Method formate): Retention time 0.88 min, MH⁺=449

### Example 17

### 2-[(1-Methylethyl)oxy]-5-(5-{5-methyl-2-[2-(methyloxy)ethyl]-1,2,3,4-tetrahydro-6-isoquinolinyl}-1,3,4-thiadiazol-2-yl)benzonitrile hydrochloride

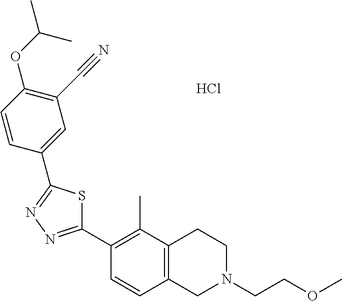

A mixture of 2-[(1-methylethyl)oxy]-5-[3-(5-methyl-1,2,3,4-tetrahydro-6-isoquinolinyl)-1,3,4-thiadiazol-2-yl]benzonitrile hydrochloride (Preparation 34) (100 mg, 0.23 mmol), 2-bromoethyl methyl diethyl ether (24 μl, 0.26 mmol) and potassium carbonate (71 mg, 0.52 mmol) in dry DMF (3 ml) was stirred at 60° C. overnight, cooled to room temperature and diluted with ethyl acetate (10 ml). The mixture was washed twice with water then brine, dried and concentrated. Purification of the residue by chromatography (ethyl acetate/isohexane: 50%) followed by purification by MDAP (Method formate) gave a residue which was dissolved in diethyl ether and treated with hydrogen chloride in diethyl ether (1M, 0.1 ml). The product was filtered off give 2-[(1-methylethyl)oxy]-5-(5-{5-methyl-2-[2-(methyloxy)ethyl]-1,2,3,4-tetrahydro-6-isoquinolinyl}-1,3,4-thiadiazol-2-yl)benzonitrile hydrochloride (30 mg, 26%) as a pale yellow solid

LCMS (Method formate): Retention time 0.92 min, MH⁺=449

### Example 18

### 5-{5-[2-(2,3-Dihydroxypropyl)-5-methyl-1,2,3,4-tetrahydro-6-isoquinolinyl]-1,3,4-thiadiazol-2-yl}-2-[(1-methylethyl)oxy]benzonitrile

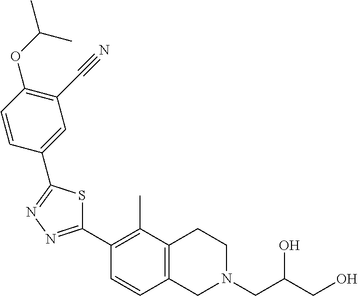

Glacial acetic acid (14 mg, 14 μl, 0.25 mmol) was added to a solution of 2-[(1-methylethyl)oxy]-5-[3-(5-methyl-1,2,3,4-tetrahydro-6-isoquinolinyl)-1,3,4-thiadiazol-2-yl]benzonitrile hydrochloride (Preparation 34) (100 mg, 0.23 mmol) in THF (3 ml) and 1,2-dichloroethane (3 ml) followed by DL-glyceraldehyde (122 mg, 1.2 mmol). The mixture was stirred at room temperature for 1 h, and then sodium triacetoxyborohydride (248 mg, 1.2 mmol) was added. The resulting mixture was stirred at room temperature overnight. A further portion of sodium triacetoxyborohydride (100 mg) was added and stirring continued for 24 h. A further portion of sodium triacetoxyborohydride (100 mg) was added and stirring continued for 6 h. The mixture was treated with saturated sodium hydrogen carbonate (10 ml) and extracted with ethyl acetate (3×10 ml). The combined organic phases were dried and concentrated. Purification of the residue by chromatography (methanol/DCM, 15%) followed by trituration with diethyl ether gave 5-{5-[2-(2,3-dihydroxypropyl)-5-methyl-1,2,3,4-tetrahydro-6-isoquinolinyl]-1,3,4-thiadiazol-2-yl}-2-[(1-methylethyl)oxy]benzonitrile (33 mg, 30%) as a light brown solid.

LCMS (Method formate): Retention time 0.88 min, [M−H]=463 (weak)

### Example 19

### 5-{3-[3-(2-Hydroxyethyl)-2,3,4,5-tetrahydro-1H-3-benzazepin-7-yl]-1,2,4-oxadiazol-5-yl}-2-[(1-methylethyl)oxy]benzonitrile

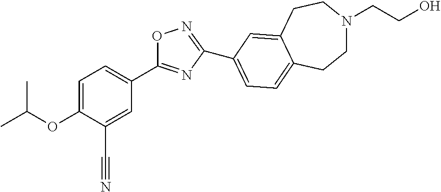

2-[(1-Methylethyl)oxy]-5-[3-(2,3,4,5-tetrahydro-1H-3-benzazepin-7-yl)-1,2,4-oxadiazol-5-yl]benzonitrile hydrochloride (Preparation 43) (100 mg), 2-iodoethanol (0.025 ml, 0.32 mmol) and potassium carbonate (111 mg, 0.80 mmol) were dissolved in DMF (2 ml) and the resulting mixture was stirred at 80° C. for 1.5 h. Water (15 ml) was added and the mixture was extracted with diethyl ether (2×15 ml). The combined organic phases were dried (Na₂SO₄) and concentrated in vacuo. Purification of the residue by flash chromatography (DCM/methanol: 0 to 10% gradient) gave 5-{3-[3-(2-hydroxyethyl)-2,3,4,5-tetrahydro-1H-3-benzazepin-7-yl]-1,2,4-oxadiazol-5-yl}-2-[(1-methylethyl)oxy]benzonitrile (26 mg) as a brown oil.

LCMS (Method formate, 5 min): Retention time 1.99 min, MH⁺=419

### Example 20

### 5-{3-[3-(3-Hydroxypropyl)-2,3,4,5-tetrahydro-1H-3-benzazepin-7-yl]-1,2,4-oxadiazol-5-yl}-2-[(1-methylethyl)oxy]benzonitrile

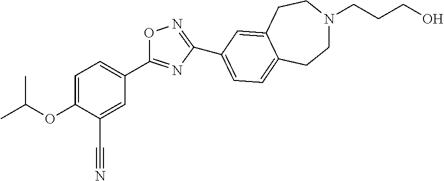

A mixture of 2-[(1-methylethyl)oxy]-5-[3-(2,3,4,5-tetrahydro-1H-3-benzazepin-7-yl)-1,2,4-oxadiazol-5-yl]benzonitrile hydrochloride (Preparation 43) (110 mg), 3-iodo-1-propanol (0.031 ml, 0.32 mmol) and potassium carbonate (111 mg, 0.80 mmol) in DMF (2 ml) was stirred at 80° C. for 1.5 h. Water (15 ml) was added and the mixture was extracted with ethyl acetate (2×20 ml). The combined organic phases were dried (Na₂SO₄) and concentrated in vacuo. Purification of the residue by MDAP (Method formate) gave 5-{3-[3-(3-hydroxypropyl)-2,3,4,5-tetrahydro-1H-3-benzazepin-7-yl]-1,2,4-oxadiazol-5-yl}-2-[(1-methylethyl)oxy]benzonitrile (30 mg) as an off white solid.

LCMS (Method formate): Retention time 0.86 min, MH⁺=433

### Example 21

### 2-[(1-Methylethyl)oxy]-5-(3-{3-[2-(methyloxy)ethyl]-2,3,4,5-tetrahydro-1H-3-benzazepin-7-yl}-1,2,4-oxadiazol-5-yl)benzonitrile

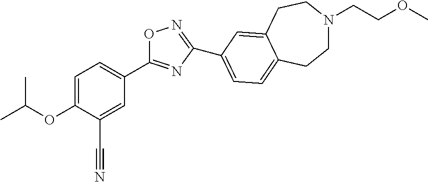

A mixture of 2-[(1-methylethyl)oxy]-5-[3-(2,3,4,5-tetrahydro-1H-3-benzazepin-7-yl)-1,2,4-oxadiazol-5-yl]benzonitrile hydrochloride (Preparation 43) (110 mg), 2-bromoethyl methyl diethyl ether (0.038 ml, 0.40 mmol) and potassium carbonate (111 mg, 0.80 mmol) in DMF (2 ml) was stirred at 80° C. for 1.5 h. Water (15 ml) was added and the aqueous phase was extracted with ethyl acetate (2×20 ml). The combined organic phases were dried (Na₂SO₄) and concentrated in vacuo. Purification of the residue by flash chromatography (methanol/DCM, 0-10%) gave 2-[(1-methylethyl)oxy]-5-(3-{3-[2-(methyloxy)ethyl]-2,3,4,5-tetrahydro-1H-3-benzazepin-7-yl}-1,2,4-oxadiazol-5-yl)benzonitrile (62 mg,) as a brown oil.

LCMS (Method formate): Retention time 0.96 min, MH⁺=433

### Example 22

### 3-[7-(5-{3-Cyano-4-[(1-methylethyl)oxy]phenyl}-1,2,4-oxadiazol-3-yl)-1,2,4,5-tetrahydro-3H-3-benzazepin-3-yl]propanamide

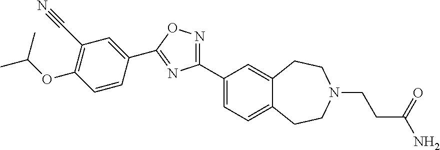

2-[(1-Methylethyl)oxy]-5-[3-(2,3,4,5-tetrahydro-1H-3-benzazepin-7-yl)-1,2,4-oxadiazol-5-yl]benzonitrile hydrochloride (Preparation 43) (110 mg, 0.27 mmol), 2-propenamide (29 mg, 0.40 mmol) and DBU (0.12 ml, 0.80 mmol) were dissolved in acetonitrile (2 ml) and the resulting mixture was stirred at 80° C. for 1 h and concentrated in vacuo. The residue was diluted with water (10 ml) and the mixture was extracted with diethyl ether (2×15 ml). The combined organic phases were and concentrated to give 3-[7-(5-{3-cyano-4-[(1-methylethyl)oxy]phenyl}-1,2,4-oxadiazol-3-yl)-1,2,4,5-tetrahydro-3H-3-benzazepin-3-yl]propanamide (110 mg, 92%) as a white solid.

LCMS (Method formate): Retention time 0.89 min, MH⁺=446

### Example 23

### 3-[7-(5-{3-Cyano-4-[(1-methylethyl)oxy]phenyl}-1,2,4-oxadiazol-3-yl)-1,2,4,5-tetrahydro-3H-3-benzazepin-3-yl]-N,N-dimethylpropanamide

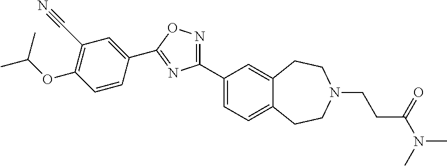

2-[(1-Methylethyl)oxy]-5-[3-(2,3,4,5-tetrahydro-1H-3-benzazepin-7-yl)-1,2,4-oxadiazol-5-yl]benzonitrile hydrochloride (Preparation 43) (110 mg, 0.27 mmol), N,N-dimethyl-2-propenamide (40 mg, 0.40 mmol) and DBU (0.12 ml, 0.80 mmol) were dissolved in acetonitrile (2 ml) and the resulting mixture was heated at 80° C. for 1 h and concentrated in vacuo. The residue was diluted with water and the mixture extracted with diethyl ether (2×15 ml). The combined organic phases were and concentrated to give 3-[7-(5-{3-cyano-4-[(1-methylethyl)oxy]phenyl}-1,2,4-oxadiazol-3-yl)-1,2,4,5-tetrahydro-3H-3-benzazepin-3-yl]-N,N-dimethylpropanamide (84 mg, 66%) as an off-white solid.

LCMS (Method formate): Retention time 0.91 min, MH⁺=474

### Example 24

### 4-[7-(5-{3-Cyano-4-[(1-methylethyl)oxy]phenyl}-1,2,4-oxadiazol-3-yl)-1,2,4,5-tetrahydro-3H-3-benzazepin-3-yl]-N-methylbutanamide

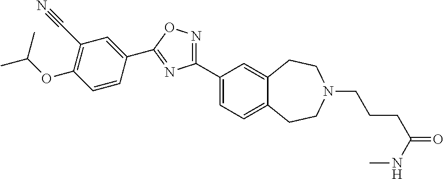

4-[7-(5-{3-Cyano-4-[(1-methylethyl)oxy]phenyl}-1,2,4-oxadiazol-3-yl)-1,2,4,5-tetrahydro-3H-3-benzazepin-3-yl]butanoic acid sodium salt (Preparation 45) (120 mg, 0.25 mmol), triethylamine (0.069 ml, 0.50 mmol), methylamine (2M solution in THF, 0.25 ml, 0.50 mmol), EDC (57.1 mg, 0.30 mmol) and HOBT (45.6 mg, 0.30 mmol) were dissolved in DMF (2 ml) and stirred at room temperature for 24 h and at 40° C. for another 24 h. Saturated Sodium hydrogen carbonate (10 ml) was added and the mixture extracted with ethyl acetate (3×10 ml). The combined organic phases were washed with brine, dried (Na₂SO₄) and concentrated. Purification of the residue by flash chromatography (methanol/DCM, 0-10%) gave 4-[7-(5-{3-cyano-4-[(1-methylethyl)oxy]phenyl}-1,2,4-oxadiazol-3-yl)-1,2,4,5-tetrahydro-3H-3-benzazepin-3-yl]-N-methylbutanamide (31 mg, 26%) as a colourless oil.

LCMS (Method formate): Retention time 0.93 min, MH⁺=474

### Example 25

### 4-[7-(5-{3-Cyano-4-[(1-methylethyl)oxy]phenyl}-1,2,4-oxadiazol-3-yl)-1,2,4,5-tetrahydro-3H-3-benzazepin-3-yl]butanamide

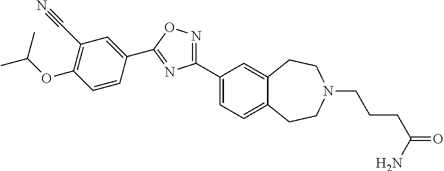

4-[7-(5-{3-Cyano-4-[(1-methylethyl)oxy]phenyl}-1,2,4-oxadiazol-3-yl)-1,2,4,5-tetrahydro-3H-3-benzazepin-3-yl]butanoic acid sodium salt (Preparation 45) (120 mg, 0.25 mmol), triethylamine (0.069 ml, 0.50 mmol), EDC (57 mg, 0.30 mmol) and HOBT ammonium salt (50 mg, 0.37 mmol) were dissolved in DMF (2 ml) and stirred for 24 h. Saturated Sodium hydrogen carbonate (10 ml) was added and the mixture extracted with ethyl acetate (3×10 ml). The combined organic phases were washed with brine, dried (Na₂SO₄) and concentrated. Purification of the residue by flash chromatography (methanol/DCM, 0-10%) gave 4-[7-(5-{3-cyano-4-[(1-methylethyl)oxy]phenyl}-1,2,4-oxadiazol-3-yl)-1,2,4,5-tetrahydro-3H-3-benzazepin-3-yl]butanamide (75 mg, 66%) as an off-white solid.

LCMS (Method formate): Retention time 0.86 min, MH⁺=460

### Example 26

### 4-[7-(5-{3-Cyano-4-[(1-methylethyl)oxy]phenyl}-1,2,4-oxadiazol-3-yl)-1,2,4,5-tetrahydro-3H-3-benzazepin-3-yl]-N,N-dimethylbutanamide

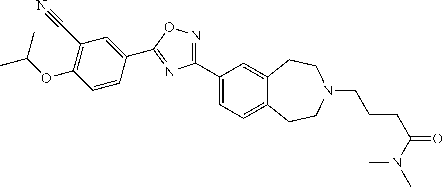

4-[7-(5-{3-Cyano-4-[(1-methylethyl)oxy]phenyl}-1,2,4-oxadiazol-3-yl)-1,2,4,5-tetrahydro-3H-3-benzazepin-3-yl]butanoic acid sodium salt (Preparation 45) (120 mg, 0.25 mmol), HOBT (46 mg, 0.30 mmol), EDC (57 mg, 0.30 mmol), triethylamine (0.069 ml, 0.50 mmol) and dimethylamine (2M solution in THF, 0.248 ml, 0.50 mmol) were dissolved in DMF (2 ml) and stirred at room temperature for 24 h and at 40° C. for a further 24 h. Saturated sodium hydrogen carbonate (10 ml) was added and the aqueous phase was extracted with ethyl acetate (3×10 ml). The combined organic phases were washed with brine, dried (Na₂SO₄) and concentrated. Purification of the residue by flash chromatography (methanol/DCM, 0-10%) gave 4-[7-(5-{3-cyano-4-[(1-methylethyl)oxy]phenyl}-1,2,4-oxadiazol-3-yl)-1,2,4,5-tetrahydro-3H-3-benzazepin-3-yl]-N,N-dimethylbutanamide (15 mg, 13%) as an off white solid.

LCMS (Method formate): Retention time 0.99 min, MH⁺=488

### Example 27

### 5-{3-[3-(2,3-Dihydroxypropyl)-2,3,4,5-tetrahydro-1H-3-benzazepin-7-yl]-1,2,4-oxadiazol-5-yl}-2-[(1-methylethyl)oxy]benzonitrile

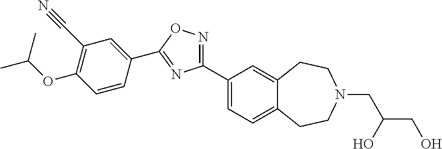

Glacial acetic acid (15 μl, 0.26 mmol) was added to a solution of 2-[(1-methylethyl)oxy]-5-[3-(2,3,4,5-tetrahydro-1H-3-benzazepin-7-yl)-1,2,4-oxadiazol-5-yl]benzonitrile hydrochloride (Preparation 43) (100 mg, 0.24 mmol) in THF (3 ml) and 1,2-dichloroethane (3 ml) followed by DL-glyceraldehyde (127 mg, 1.2 mmol) and the resulting mixture was stirred at room temperature for 1 h. Triacetoxyborohydride (258 mg, 1.2 mmol) was added and the reaction stirred at ambient temperature overnight. A further portion of triacetoxyborohydride (100 mg) was added and the mixture stirred for 24 h. A third portion of triacetoxyborohydride (100 mg) was added and stirring continued for 6 h. The mixture was treated with saturated Sodium hydrogen carbonate (10 ml) and extracted with ethyl acetate (3×10 ml). The combined organic phases were dried and concentrated. Purification of the residue by flash chromatography (methanol/DCM, 15%) followed by trituration with diethyl ether gave 5-{3-[3-(2,3-dihydroxypropyl)-2,3,4,5-tetrahydro-1H-3-benzazepin-7-yl]-1,2,4-oxadiazol-5-yl}-2-[(1-methylethyl)oxy]benzonitrile (53 mg, 49%) as a light brown solid.

LCMS (Method formate): Retention time 0.94 min, MH⁺=449 (weak)

### Example 28

### 5-{5-[3-(2-Hydroxyethyl)-2,3,4,5-tetrahydro-1H-3-benzazepin-7-yl]-1,3,4-thiadiazol-2-yl}-2-[(1-methylethyl)oxy]benzonitrile hydrochloride

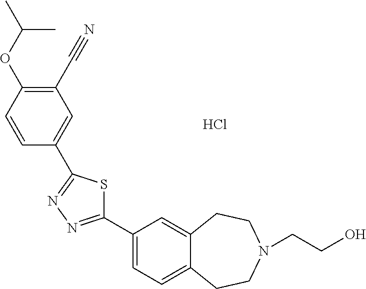

A mixture of 2-[(1-methylethyl)oxy]-5-[3-(2,3,4,5-tetrahydro-1H-3-benzazepin-7-yl)-1,3,4-thiadiazol-2-yl]benzonitrile trifluoroacetic salt (Preparation 47) (60 mg, 0.12 mmol), 2-bromoethanol (0.013 ml, 0.18 mmol) and potassium carbonate (49 mg, 0.36 mmol) in acetone (1.5 ml) was stirred at 90° C. for 20 min (microwave), filtered and concentrated in vacuo. The residue was loaded onto an SCX cartridge and eluted with methanol then with a 10% w/w NH₃ solution in methanol. The combined methanolic fractions were concentrated and the residue dissolved in DCM (1 ml) then treated with a hydrogen chloride solution in diethyl ether (0.1 ml). The resulting mixture was concentrated to give 5-{5-[3-(2-hydroxyethyl)-2,3,4,5-tetrahydro-1H-3-benzazepin-7-yl]-1,3,4-thiadiazol-2-yl}-2-[(1-methylethyl)oxy]benzonitrile hydrochloride (25 mg, 42%) as a colourless solid.

LCMS (Method formate): Retention time 0.81 min, MH⁺=435

### Example 29

### 5-{5-[3-(3-Hydroxypropyl)-2,3,4,5-tetrahydro-1H-3-benzazepin-7-yl]-1,3,4-thiadiazol-2-yl}-2-[(1-methylethyl)oxy]benzonitrile

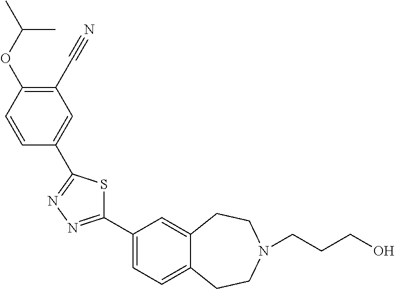

A mixture of 2-[(1-methylethyl)oxy]-5-[3-(2,3,4,5-tetrahydro-1H-3-benzazepin-7-yl)-1,3,4-thiadiazol-2-yl]benzonitrile trifluoroacetic salt (Preparation 47) (60 mg, 0.12 mmol), 3-bromo-1-propanol (0.016 ml, 0.18 mmol) and potassium carbonate (49 mg, 0.36 mmol) in acetone (1.5 ml) was stirred at 90° C. for 20 min (microwave), filtered and concentrated. The residue was loaded onto an SCX cartridge (5 g) and eluted with methanol then with a 10% w/w ammonia solution in methanol. The combined methanolic fractions were concentrated and the residue triturated with diethyl ether to give 5-{5-[3-(3-hydroxypropyl)-2,3,4,5-tetrahydro-1H-3-benzazepin-7-yl]-1,3,4-thiadiazol-2-yl}-2-[(1-methylethyl)oxy]benzonitrile (25 mg, 42%) as a colourless solid.

LCMS (Method formate): Retention time 0.80 min, MH⁺=449

### Example 30

### 2-[(1-Methylethyl)oxy]-5-(5-{3-[2-(methyloxy)ethyl]-2,3,4,5-tetrahydro-1H-3-benzazepin-7-yl}-1,3,4-thiadiazol-2-yl)benzonitrile hydrochloride

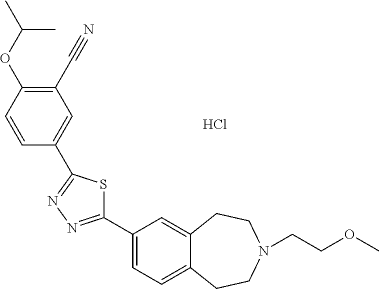

A mixture of 2-[(1-methylethyl)oxy]-5-[3-(2,3,4,5-tetrahydro-1H-3-benzazepin-7-yl)-1,3,4-thiadiazol-2-yl]benzonitrile trifluoroacetic salt (Preparation 47) (60 mg, 0.12 mmol), 2-bromoethyl methyl diethyl ether (25 mg, 0.18 mmol) and potassium carbonate (49 mg, 0.36 mmol) in acetone (1.5 ml) was stirred at 90° C. for 20 min (microwave), filtered and concentrated. The residue was loaded onto an SCX (5 g) cartridge and eluted with methanol then with a 10% w/w ammonia solution in methanol. The combined methanolic fractions were concentrated, the residue dissolved in DCM (1 ml) and treated with hydrogen chloride solution in diethyl ether (0.1 ml). The resulting mixture was concentrated to give 2-[(1-methylethyl)oxy]-5-(5-{3-[2-(methyloxy)ethyl]-2,3,4,5-tetrahydro-1H-3-benzazepin-7-yl}-1,3,4-thiadiazol-2-yl)benzonitrile hydrochloride (30 mg, 52%) as a colourless solid.

LCMS (Method formate): Retention time 0.82 min, MH⁺=449

### Example 31

### 2-[6-(5-{3-Cyano-4-[(1-methylethyl)oxy]phenyl}-1,2,4-oxadiazol-3-yl)-1,2,4,5-tetrahydro-3H-3-benzazepin-3-yl]acetamide

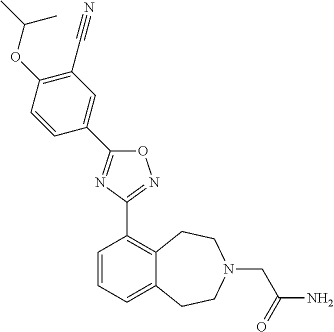

A mixture of 2-[(1-methylethyl)oxy]-5-[3-(2,3,4,5-tetrahydro-1H-3-benzazepin-6-yl)-1,2,4-oxadiazol-5-yl]benzonitrile trifluoroacetic acid salt (Preparation 52) (69 mg, 0.141 mmol), 2-bromoacetamide (19 mg, 0.14 mmol) and potassium carbonate (59 mg, 0.42 mmol) in acetone (1.5 ml) was stirred at 90° C. for 20 min (microwave), the cooled mixture was filtered and the filtrate concentrated. The residue was stirred in methanol (1 ml, the precipitate formed filtered off and dried to give 2-[6-(5-{3-cyano-4-[(1-methylethyl)oxy]phenyl}-1,2,4-oxadiazol-3-yl)-1,2,4,5-tetrahydro-3H-3-benzazepin-3-yl]acetamide (35 mg, 57%) as a colourless solid.

LCMS (Method formate): Retention time 0.88 min, MH⁺=432

### Example 32

### 3-[6-(5-{3-Cyano-4-[(1-methylethyl)oxy]phenyl}-1,2,4-oxadiazol-3-yl)-1,2,4,5-tetrahydro-3H-3-benzazepin-3-yl]propanamide

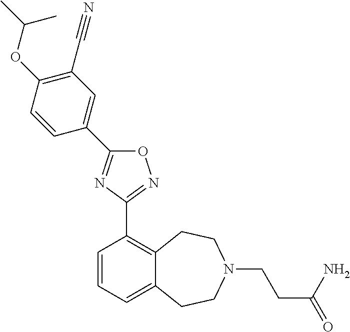

A mixture of 2-[(1-methylethyl)oxy]-5-[3-(2,3,4,5-tetrahydro-1H-3-benzazepin-6-yl)-1,2,4-oxadiazol-5-yl]benzonitrile trifluoroacetic acid salt (Preparation 52) (69 mg, 0.14 mmol), 3-bromopropanamide (32 mg, 0.21 mmol) and potassium carbonate (59 mg, 0.42 mmol) in acetone (1.5 ml) was stirred at 90° C. for 20 min (microwave) the cooled reaction was filtered and the filtrate concentrated. The residue was stirred in methanolmethanol (1 ml), the precipitate formed filtered off and dried to give 3-[6-(5-{3-cyano-4-[(1-methylethyl)oxy]phenyl}-1,2,4-oxadiazol-3-yl)-1,2,4,5-tetrahydro-3H-3-benzazepin-3-yl]propanamide (33 mg, 51%) as a colourless solid.

LCMS (Method formate): Retention time 0.88 min, MH⁺=446

### Example 33

### 5-{3-[3-(3-Hydroxypropyl)-2,3,4,5-tetrahydro-1H-3-benzazepin-6-yl]-1,2,4-oxadiazol-5-yl}-2-[(1-methylethyl)oxy]benzonitrile

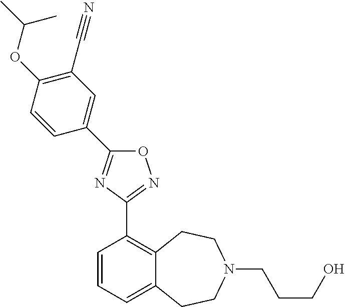

A mixture of 2-[(1-methylethyl)oxy]-5-[3-(2,3,4,5-tetrahydro-1H-3-benzazepin-6-yl)-1,2,4-oxadiazol-5-yl]benzonitrile trifluoroacetic acid salt (Preparation 52) (65 mg, 0.13 mmol), 3-bromo-1-propanol (0.012 ml, 0.13 mmol) and potassium carbonate (55 mg, 0.40 mmol) in acetone (1.5 ml) was stirred at 90° C. for 20 min (microwave) the cooled reaction was filtered and the filtrate concentrated. The residue was loaded on an SCX cartridge and eluted with methanol then with a ammonia in methanol (2M). The combined methanolic ammonia fractions were concentrated to give 5-{3-[3-(3-hydroxypropyl)-2,3,4,5-tetrahydro-1H-3-benzazepin-6-yl]-1,2,4-oxadiazol-5-yl}-2-[(1-methylethyl)oxy]benzonitrile (30 mg, 50%) as colourless foam.

LCMS (Method formate): Retention time 0.86 min, MH⁺=433

### Example 34

### 5-{3-[3-(2-Hydroxyethyl)-2,3,4,5-tetrahydro-1H-3-benzazepin-6-yl]-1,2,4-oxadiazol-5-yl}-2-[(1-methylethyl)oxy]benzonitrile

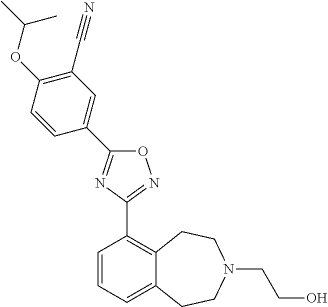

A mixture of 2-[(1-methylethyl)oxy]-5-[3-(2,3,4,5-tetrahydro-1H-3-benzazepin-6-yl)-1,2,4-oxadiazol-5-yl]benzonitrile trifluoroacetic acid salt (Preparation 52) (63 mg, 0.13 mmol), 2-bromoethanol (0.014 ml, 0.19 mmol) and potassium carbonate (54 mg, 0.39 mmol) in acetone (1.5 ml) was stirred at 90° C. for 20 min (microwave) the cooled reaction mixture was filtered and the filtrate concentrated. The residue was loaded on an SCX cartridge and eluted with methanol then with ammonia in methanol. The combined methanolic ammonia fractions were concentrated to give 5-{3-[3-(2-hydroxyethyl)-2,3,4,5-tetrahydro-1H-3-benzazepin-6-yl]-1,2,4-oxadiazol-5-yl}-2-[(1-methylethyl)oxy]benzonitrile (33 mg, 61%) as a colourless foam.

LCMS (Method formate): Retention time 0.82 min, MH⁺=419

### Example 35

### 5-{3-[3-(2,3-Dihydroxypropyl)-2,3,4,5-tetrahydro-1H-3-benzazepin-6-yl]-1,2,4-oxadiazol-5-yl}-2-[(1-methylethyl)oxy]benzonitrile hydrochloride

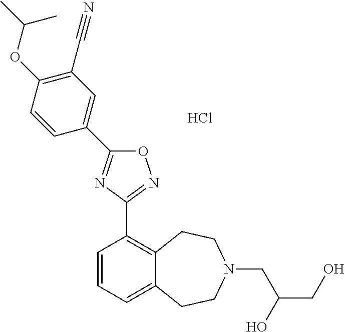

A mixture of 2-[(1-methylethyl)oxy]-5-[3-(2,3,4,5-tetrahydro-1H-3-benzazepin-6-yl)-1,2,4-oxadiazol-5-yl]benzonitrile trifluoroacetic acid salt (Preparation 52) (65 mg, 0.13 mmol) and DL-glyceraldehyde (24 mg, 0.27 mmol) in DCM (2 ml) was stirred at room temperature overnight. Sodium triacetoxyborohydride (56 mg, 0.27 mmol) was then added and the mixture stirred for 5 h. DL-glyceraldehyde (120 mg, 1.3 mmol) and acetic acid (8 μl, 0.13 mmol) were added and the resulting mixture stirred for 5 h. Sodium triacetoxyborohydride (100 mg, 0.47 mmol) was added and the mixture stirred overnight then concentrated. Purification of the residue by flash chromatography on silica gel (2M ammonia in methanol/DCM, 15-30%) and concentration of the combined product fractions gave a buff coloured foam. This was dissolved in methanol (1 ml) and treated with hydrogen chloride in diethyl ether (1M, 0.2 ml). The solvent was concentrated and the residue triturated with diethyl ether to give 5-{3-[3-(2,3-dihydroxypropyl)-2,3,4,5-tetrahydro-1H-3-benzazepin-6-yl}-1,2,4-oxadiazol-5-yl]-2-[(1-methylethyl)oxy]benzonitrile hydrochloride (40 mg, 47%) as a buff-colored solid.

LCMS (Method formate): Retention time 0.88 min, MH⁺=449

### Example 36

### 5-{5-[2-(3-Hydroxypropyl)-1,2,3,4-tetrahydro-5-isoquinolinyl]-1,3,4-thiadiazol-2-yl}-2-[(1-methylethyl)oxy]benzonitrile

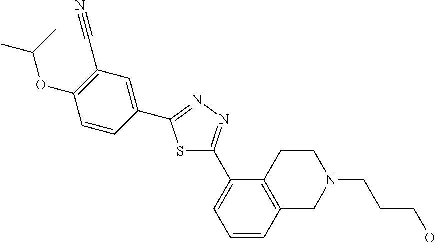

A solution of 2-[(1-methylethyl)oxy]-5-[3-(1,2,3,4-tetrahydro-5-isoquinolinyl)-1,3,4-thiadiazol-2-yl]benzonitrile hydrochloride (Preparation 56) (50 mg, 0.12 mmol) and potassium carbonate (50 mg, 0.36 mmol) in DMF (1 ml) was treated with 3-iodo-1-propanol (0.017 ml, 0.18 mmol) and the resulting mixture was stirred at 80° C. for 2.5 h. The mixture was diluted with water (10 ml) mlextracted with ethyl acetate (3×10 ml). The combined organic phases were dried (Na₂SO₄) and concentrated in vacuo. Purification of the residue by flash chromatography on silica gel (methanol/cyclohexane, 0-5%) gave 5-{5-[2-(3-hydroxypropyl)-1,2,3,4-tetrahydro-5-isoquinolinyl]-1,3,4-thiadiazol-2-yl}-2-[(1-methylethyl)oxy]benzonitrile (9 mg, 17%) as an orange oil.

LCMS (Method formate): Retention time 0.83 min, MH⁺=435

### Example 37

### 2-[(1-Methylethyl)oxy]-5-(5-{2-[2-(methyloxy)ethyl]-1,2,3,4-tetrahydro-5-isoquinolinyl}-1,3,4-thiadiazol-2-yl)benzonitrile

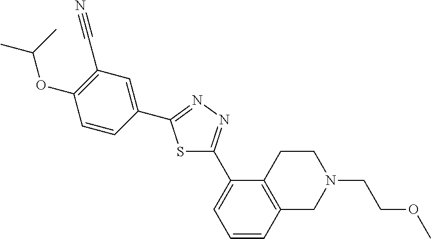

A solution of 2-[(1-methylethyl)oxy]-5-[3-(1,2,3,4-tetrahydro-5-isoquinolinyl)-1,3,4-thiadiazol-2-yl]benzonitrile hydrochloride (Preparation 56) (50 mg, 0.12 mmol) and potassium carbonate (50 mg, 0.36 mmol) in DMF (1 ml) was treated with 1-bromo-2-(methyloxy)ethane (0.017 ml, 0.18 mmol) and the mixture stirred at 80° C. for 2 h. The mixture was diluted with water (10 ml) and extracted with ethyl acetate (3×10 ml). The combined organic phases were dried (Na₂SO₄) and concentrated in vacuo. Purification of the residue by flash chromatography (methanol/cyclohexane, 0-5%) gave 2-[(1-methylethyl)oxy]-5-(5-{2-[2-(methyloxy)ethyl]-1,2,3,4-tetrahydro-5-isoquinolinyl}-1,3,4-thiadiazol-2-yl)benzonitrile (40 mg, 75%) as an orange oil.

LCMS (Method formate): Retention time 0.90 min, MH⁺=435

### Example 38

### 5-{5-[2-(2-hydroxyethyl)-1,2,3,4-tetrahydro-5-isoquinolinyl]-1,3,4-thiadiazol-2-yl}-2-[(1-methylethyl)oxy]benzonitrile

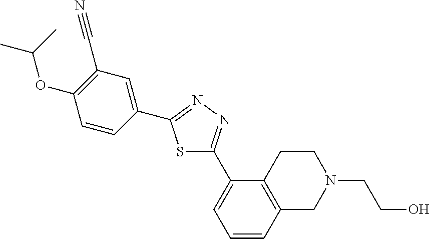

A solution of 2-[(1-methylethyl)oxy]-5-[3-(1,2,3,4-tetrahydro-5-isoquinolinyl)-1,3,4-thiadiazol-2-yl]benzonitrile hydrochloride (Preparation 56) (50 mg, 0.12 mmol) and potassium carbonate (50 mg, 0.36 mmol) in DMF (1 ml) was treated with 2-iodoethanol (0.014 ml, 0.18 mmol) and the mixture stirred at 80° C. for 2 h. The mixture was diluted with water (10 ml) and extracted with ethyl acetate (3×10 ml). The combined organic phases were dried (Na₂SO₄) and concentrated in vacuo. Purification of the residue by flash chromatography (cyclohexane/methanol: 0 to 5% gradient) gave 5-{5-[2-(2-hydroxyethyl)-1,2,3,4-tetrahydro-5-isoquinolinyl]-1,3,4-thiadiazol-2-yl}-2-[(1-methylethyl)oxy]benzonitrile (36 mg, 71%) as a clear oil.

LCMS (Method formate): Retention time 0.85 min, MH⁺=421

### Example 39

### 5-[3-(2-β-Alanyl-5-methyl-1,2,3,4-tetrahydro-6-isoquinolinyl)-1,2,4-oxadiazol-5-yl]-2-[(1-methylethyl)oxy]benzonitrile hydrochloride

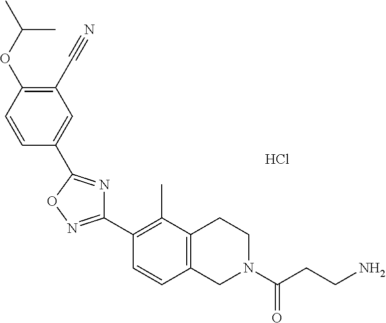

A solution of 1,1-dimethylethyl {3-[6-(5-{3-cyano-4-[(1-methylethyl)oxy]phenyl}-1,2,4-oxadiazol-3-yl)-5-methyl-3,4-dihydro-2(1H)-isoquinolinyl]-3-oxopropyl}carbamate (Preparation 57) (150 mg, 0.28 mmol) in 1,4-dioxane (1 ml) was treated with hydrogen chloride in 1,4-dioxane (4M, 1 ml) and the reaction stirred at room temperature for 7 h. diethyl ether (10 ml) was added and the mixture stirred for 30 min. The precipitate was filtered off, washed with diethyl ether and dried to give 5-[3-(2-β-alanyl-5-methyl-1,2,3,4-tetrahydro-6-isoquinolinyl)-1,2,4-oxadiazol-5-yl]-2-[(1-methylethyl)oxy]benzonitrile hydrochloride (99 mg, 75%) as a colourless solid.

LCMS (Method formate): Retention time 0.93 min, MH⁺=446

### Example 40

### 2-[(1-Methylethyl)oxy]-5-{3-[5-methyl-2-(N-methyl-□-alanyl)-1,2,3,4-tetrahydro-6-isoquinolinyl]-1,2,4-oxadiazol-5-yl}benzonitrile hydrochloride

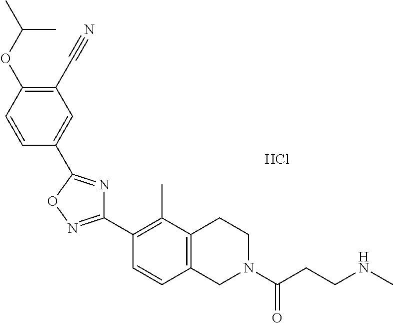

A solution of 1,1-dimethylethyl {3-[6-(5-{3-cyano-4-[(1-methylethyl)oxy]phenyl}-1,2,4-oxadiazol-3-yl)-5-methyl-3,4-dihydro-2(1H)-isoquinolinyl]-3-oxopropyl}methylcarbamate (Preparation 58) (120 mg, 0.21 mmol) in 1,4-dioxane (1 ml) was treated with hydrogen chloride in 1,4-dioxane (4M, 1 ml) and the mixture was stirred at room temperature for 7 h. Ether (10 ml) was added and the mixture stirred for 30 min. The precipitate was filtered off, washed with diethyl ether and dried to give 2-[(1-methylethyl)oxy]-5-{3-[5-methyl-2-(N-methyl-b-alanyl)-1,2,3,4-tetrahydro-6-isoquinolinyl]-1,2,4-oxadiazol-5-yl}benzonitrile hydrochloride (103 mg, 97%) as a colourless solid.

LCMS (Method formate): Retention time 0.97 min, MH⁺=460

### Example 41

### 5-{3-[2-(4-Aminobutanoyl)-5-methyl-1,2,3,4-tetrahydro-6-isoquinolinyl]-1,2,4-oxadiazol-5-yl}-2-[(1-methylethyl)oxy]benzonitrile hydrochloride

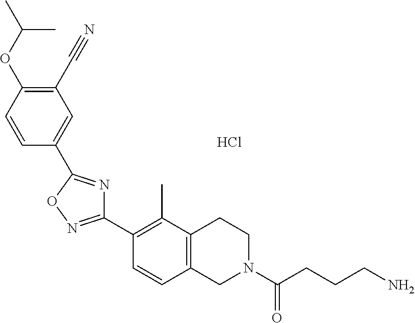

A solution of 1,1-dimethylethyl {4-[6-(5-{3-cyano-4-[(1-methylethyl)oxy]phenyl}-1,2,4-oxadiazol-3-yl)-5-methyl-3,4-dihydro-2(1H)-isoquinolinyl]-4-oxobutyl}carbamate (Preparation 59) (110 mg, 0.20 mmol) in 1,4-dioxane (1 ml) was treated with hydrogen chloride in 1,4-dioxane (4M, 1 ml) and the mixture was stirred at room temperature for 7 h. Ether (10 ml) was added and the mixture stirred for 30 min. The precipitate was filtered off, washed with diethyl ether and dried to give 5-{3-[2-(4-aminobutanoyl)-5-methyl-1,2,3,4-tetrahydro-6-isoquinolinyl]-1,2,4-oxadiazol-5-yl}-2-[(1-methylethyl)oxy]benzonitrile hydrochloride (96 mg, 98%) as a colourless solid.

LCMS (method formate): Retention time 0.94 min, MH⁺=460

### Example 42

### 5-[3-(2-Glycyl-5-methyl-1,2,3,4-tetrahydro-6-isoquinolinyl)-1,2,4-oxadiazol-5-yl]-2-[(1-methylethyl)oxy]benzonitrile hydrochloride

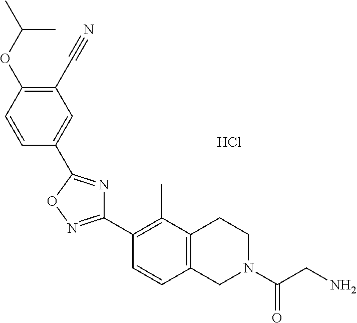

To a solution of 1,1-dimethylethyl {2-[6-(5-{3-cyano-4-[(1-methylethyl)oxy]phenyl}-1,2,4-oxadiazol-3-yl)-5-methyl-3,4-dihydro-2(1H)isoquinolinyl]-2-oxoethyl}carbamate (Preparation 60) (200 mg, 0.38 mmol) in 1,4-dioxane (3 ml) at room temperature under nitrogen was added hydrogen chloride solution (4N, 3 ml, 12 mmol) and the mixture was stirred at this temperature for 5 h then concentrated. The residue was co-evaporated with diethyl ether and dried under vacuum to give 5-[3-(2-glycyl-5-methyl-1,2,3,4-tetrahydro-6-isoquinolinyl)-1,2,4-oxadiazol-5-yl]-2-[(1-methylethyl)oxy]benzonitrile hydrochloride (159 mg, 90%) as a white solid.

LCMS (Method HpH): Retention time 1.10 min, MH⁺=432

### Example 43

### 3-[6-(5-{3-cyano-4-[(1-methylethyl)oxy]phenyl}-1,2,4-oxadiazol-3-yl)-5-methyl-3,4-dihydro-2(1H)-isoquinolinyl]-N-methylpropanamide

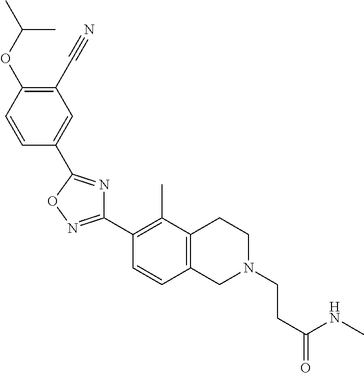

A mixture of 3-[6-(5-{3-cyano-4-[(1-methylethyl)oxy]phenyl}-1,2,4-oxadiazol-3-yl)-5-methyl-3,4-dihydro-2(1H)-isoquinolinyl]propanoic acid sodium salt (Preparation 27) (150 mg, 0.32 mmol), N-ethylmorpholine (81 μl, 0.64 mmol), N-hydroxybenzotriazole hydrate (59 mg, 0.38 mmol), and EDC (74 mg, 0.38 mmol) in DMF (3 ml) was stirred at room temperature for 30 min. Methylamine in THF (2M, 1 ml, 2 mmol) was added and the reaction mixture stirred at room temperature overnight. The reaction mixture was diluted with ethyl acetate (10 ml) and washed with sataturated sodium hydrogen carbonate (2×10 ml) and brine. The organic phase was dried and evaporated. The residue was partially purified by chromatography (methanol/DCM, 10%) and the isolated product further purified by MDAP (Method formate) gave 3-[6-(5-{3-cyano-4-[(1-methylethyl)oxy]phenyl}-1,2,4-oxadiazol-3-yl)-5-methyl-3,4-dihydro-2(1H)-isoquinolinyl]-N-methylpropanamide as a colourless solid (30 mg)

LCMS (Method formate): Retention time 0.92 min, MH⁺=460

### Example 44

### 4-[7-(5-{3-cyano-4-[(1-methylethyl)oxy]phenyl}-1,2,4-oxadiazol-3-yl)-1,2,4,5-tetrahydro-3H-3-benzazepin-3-yl]-N-ethylbutanamide

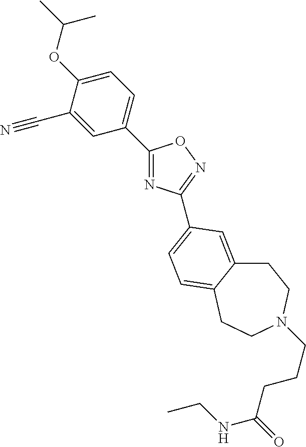

N-Ethylmorpholine (100 mg, 110 μl, 0.87 mmol) was added to a stirred solution of 447-(5-{3-cyano-4-[(1-methylethyl)oxy]phenyl}-1,2,4-oxadiazol-3-yl)-1,2,4,5-tetrahydro-3H-3-benzazepin-3-yl]butanoic acid hydrochloride (Preparation 45) (200 mg) in dry THF (5 ml) followed by isobutyl chloroformate (68 μl, 0.52 mmol). The reaction mixture was stirred at room temperature for 5 min then treated with aqueous ethylamine (70%, 1 ml). The reaction mixture was stirred at room temperature overnight. The reaction mixture was diluted with ethyl acetate (10 ml) and washed with saturated sodium hydrogen carbonate, water and brine. The organic phase was dried and evaporated. Purification by MDAP (Method formate) gave the required compound as a colourless glass (24 mg).

LCMS (Method formate): Retention time 0.98 min, MH⁺=488

### Example 45

### 3-[5-(5-{3-cyano-4-[(1-methylethyl)oxy]phenyl}-1,3,4-thiadiazol-2-yl)-3,4-dihydro-2(1H)-isoquinolinyl]-N-methylpropanamide

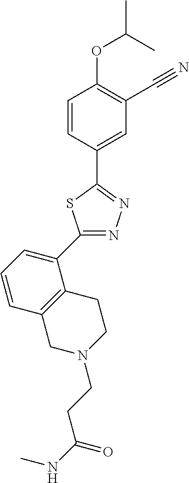

2-[(1-Methylethyl)oxy]-5-[3-(1,2,3,4-tetrahydro-5-isoquinolinyl)-1,3,4-thiadiazol-2-yl]benzonitrile hydrochloride (Preparation 56) (40 mg), N-methyl-2-propenamide (12 mg) and DBU (0.044 ml, 0.29 mmol) were dissolved in acetonitrile (1 ml) and heated to 80° C. for 1 h. The acetonitrile (1 ml) was removed under vacuum. Water (10 ml) was added and the mixture extracted using diethyl ether (2×15 ml). The organic phase was dried (Na₂SO₄) and the solvent removed under vacuum to give a crude product. The product was purified using flash chromatography (methanol/DCM, 0-10%) to give 3-[5-(5-{3-cyano-4-[(1-methylethyl)oxy]phenyl}-1,3,4-thiadiazol-2-yl)-3,4-dihydro-2(1H)-isoquinolinyl]-N-methylpropanamide as a yellow solid (27 mg).

LCMS (Method formate): Retention time 0.87 min, MH⁺=462

### Example 46

### 3-[5-(5-{3-cyano-4-[(1-methylethyl)oxy]phenyl}-1,3,4-thiadiazol-2-yl)-3,4-dihydro-2(1H)-isoquinolinyl]-N,N-dimethylpropanamide

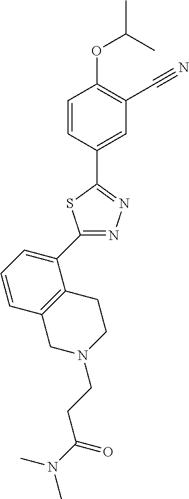

2-[(1-Methylethyl)oxy]-5-[3-(1,2,3,4-tetrahydro-5-isoquinolinyl)-1,3,4-thiadiazol-2-yl]benzonitrile hydrochloride (Preparation 56) (50 mg), N,N-dimethyl-2-propenamide (18 mg) and DBU (0.055 ml, 0.36 mmol) were dissolved in acetonitrile (1 ml) and heated to 80° C. for 1 h. The acetonitrile (1 ml) was evaporated under vacuum. Water (10 ml) was added and the mixture was extracted using diethyl ether (2×15 ml). 345-(5-{3-Cyano-4-[(1-methylethyl)oxy]phenyl}-1,3,4-thiadiazol-2-yl)-3,4-dihydro-2(1H)-isoquinolinyl]-N,N-dimethylpropanamide was isolated as a brown solid (27 mg).

LCMS (Method formate): Retention time 0.90 min, MH⁺=476

### Example 47

### 3-[6-(5-{3-cyano-4-[(1-methylethyl)oxy]phenyl}-1,3,4-thiadiazol-2-yl)-3,4-dihydro-2(1H)-isoquinolinyl]propanamide

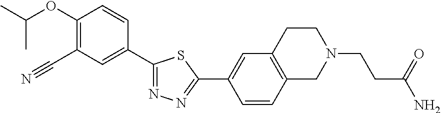

2-[(1-Methylethyl)oxy]-5-[3-(1,2,3,4-tetrahydro-6-isoquinolinyl)-1,3,4-thiadiazol-2-yl]benzonitrile (Preparation 61) (48 mg, 0.13 mmol) and acrylamide (10 mg, 0.14 mmol) were dissolved in acetonitrile (3 ml). Silica gel (500 mg, 8.3 mmol) was added and the mixture heated at 60° C. under nitrogen for 6 h and the mixture was allowed to cool to room temperature overnight. The mixture filtered through a Celite™ column, washing with DCM, methanol and ethyl acetate. This organic mixture was evaporated. The residue was dissolved in methanol/DMSO (1:1, 1 ml) and purified by MDAP (Method formate). The solvent was evaporated to give 3-[6-(5-{3-cyano-4-[(1-methylethyl)oxy]phenyl}-1,3,4-thiadiazol-2-yl)-3,4-dihydro-2(1H)-isoquinolinyl]propanamide as an off pale brown solid (33 mg).

LCMS (Method formate): Retention time 0.83 min, MH⁺=448

### Example 48

### 5-[3-(3-glycyl-2,3,4,5-tetrahydro-1H-3-benzazepin-7-yl)-1,2,4-oxadiazol-5-yl]-2-[(1-methylethyl)oxy]benzonitrile trifluoroacetate

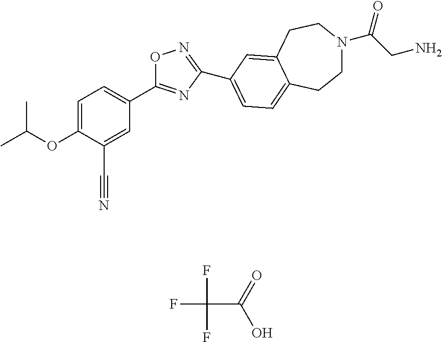

Trifluoroacetic acid (1 ml, 13 mmol) was added to 1,1-dimethylethyl {2-[7-(5-{3-cyano-4-[(1-methylethyl)oxy]phenyl}-1,2,4-oxadiazol-3-yl)-1,2,4,5-tetrahydro-3H-3-benzazepin-3-yl]-2-oxoethyl}carbamate (Preparation 65) (200 mg, 0.38 mmol) in DCM (4 ml), and the mixture was stirred at room temperature for 1 h, The reaction mixture was filtered through an aminopropyl SPE (5 g) washing with methanol in DCM (10%). The appropriate fractions were combined and evaporated in vacuo. The residue was dissolved in DMSO (2×1 ml) and purified by MDAP (x2, Method formate) The solvent was evaporated in vacuo to give 5-[3-(3-glycyl-2,3,4,5-tetrahydro-1H-3-benzazepin-7-yl)-1,2,4-oxadiazol-5-yl]-2-[(1-methylethyl)oxy]benzonitrile trifluoroacetate (116 mg).

LCMS (Method formate): Retention time 0.90 min, MH⁺=432

### Example 49

### 5-{3-[3-(2,3-dihydroxypropyl)-2,3,4,5-tetrahydro-1H-3-benzazepin-7-yl]-1,2,4-oxadiazol-5-yl}-2-[(1-methylethyl)amino]benzonitrile

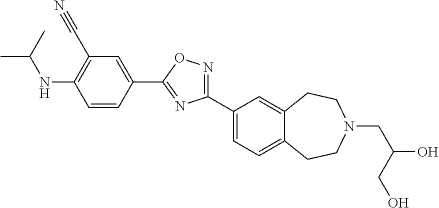

To 2-[(1-methylethyl)amino]-5-[3-(2,3,4,5-tetrahydro-1H-3-benzazepin-7-yl)-1,2,4-oxadiazol-5-yl]benzonitrile (Preparation 66) (91 mg, 0.24 mmol) in DCM (1.5 ml) and THF (1.5 ml) was added DL-glyceraldehyde (110 mg, 1.2 mmol) and acetic acid (0.015 ml, 0.26 mmol). The reaction mixture was stirred at room temperature for 10 min. Sodium triacetoxyborohydride (258 mg, 1.2 mmol) was added and the reaction mixture stirred at room temperature overnight. The reaction mixture was quenched using saturated aqueous sodium hydrogen carbonate solution (30 ml), which was then extracted with ethyl acetate (2×50 ml). The organics were combined, dried (hydrophobic frit) and reduced to dryness under a stream of nitrogen. The residue was dissolved in DMSO (2×1 ml) and purified by MDAP (Method HpH). The solvent was evaporated under a stream of nitrogen to give 5-{3-[3-(2,3-dihydroxypropyl)-2,3,4,5-tetrahydro-1H-3-benzazepin-7-yl]-1,2,4-oxadiazol-5-yl}-2-[(1-methylethyl)amino]benzonitrile (73 mg).

LCMS (Method formate): Retention time 0.93 min, MH⁺=448

### Example 50

### 5-{3-[3-(2,3-dihydroxypropyl)-2,3,4,5-tetrahydro-1H-3-benzazepin-7-yl]-1,2,4-oxadiazol-5-yl}-2-(propylamino)benzonitrile

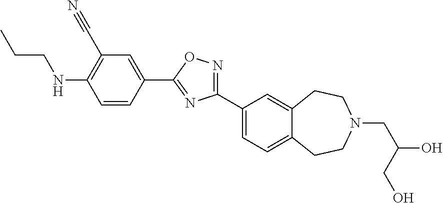

To 2-(propylamino)-5-[3-(2,3,4,5-tetrahydro-1H-3-benzazepin-7-yl)-1,2,4-oxadiazol-5-yl]benzonitrile (Preparation 70) (59 mg, 0.16 mmol) in THF (1.5 ml) and DCM (1.5 ml) was added DL-glyceraldehyde (71 mg, 0.79 mmol) and acetic acid (10 μl, 0.17 mmol).

The reaction mixture was stirred at room temperature for 10 min. Sodium triacetoxyborohydride (167 mg, 0.79 mmol) was added and the reaction mixture stirred at room temperature overnight. The reaction mixture was quenched with saturated aqueous sodium hydrogen carbonate solution (30 ml), which was extracted with ethyl acetate (2×50 ml). The organics were combined, dried (hydrophobic frit) and reduced to dryness under a stream of nitrogen. The residue was dissolved in DMSO (1 ml) and purified MDAP (Method HpH). The solvent was evaporated under a stream of nitrogen to give 5-{3-[3-(2,3-dihydroxypropyl)-2,3,4,5-tetrahydro-1H-3-benzazepin-7-yl]-1,2,4-oxadiazol-5-yl}-2-(propylamino)benzonitrile (76 mg).

LCMS (Method formate): Retention time 0.85 min, MH⁺=448.

### Example 51

### 5-{3-[3-(2,3-dihydroxypropyl)-2,3,4,5-tetrahydro-1H-3-benzazepin-7-yl]-1,2,4-oxadiazol-5-yl}-2-(propyloxy)benzonitrile

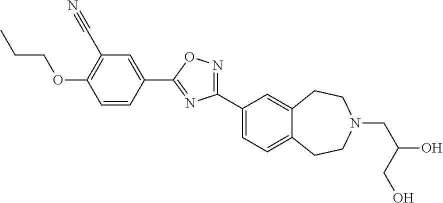

To 2-(propyloxy)-5-[3-(2,3,4,5-tetrahydro-1H-3-benzazepin-7-yl)-1,2,4-oxadiazol-5-yl]benzonitrile (Preparation 72) (51 mg, 0.14 mmol) in THF (1.5 ml) and DCM (1.5 ml) was added DL-glyceraldehyde (61 mg, 0.68 mmol) and acetic acid (8 μl, 0.14 mmol). The reaction mixture was stirred at room temperature for 10 min. Sodium triacetoxyborohydride (144 mg, 0.68 mmol) was added and the reaction mixture stirred at room temperature overnight. A further portion of sodium triacetoxyborohydride (144 mg, 0.68 mmol) was added and the reaction mixture stirred at room temperature for 4 h. Further sodium triacetoxyborohydride (144 mg, 0.68 mmol) was added and the reaction mixture was stirred at room temperature overnight. The reaction mixture was quenched using saturated aqueous sodium hydrogen carbonate solution (30 ml), which was then extracted with ethyl acetate (2×50 ml). The organics were combined, (hydrophobic frit) and reduced to dryness under a stream of nitrogen. The residue was dissolved in DMSO (1 ml) and purified by MDAP (Method HpH). The solvent was evaporated under a stream of nitrogen to give 5-{3-[3-(2,3-dihydroxypropyl)-2,3,4,5-tetrahydro-1H-3-benzazepin-7-yl]-1,2,4-oxadiazol-5-yl}-2-(propyloxy)benzonitrile (36 mg).

LCMS (Method formate): Retention time 0.87 min, MH⁺=449

### Example 52

### 5-[3-(3-glycyl-2,3,4,5-tetrahydro-1H-3-benzazepin-7-yl)-1,2,4-oxadiazol-5-yl]-2-[(1-methylethyl)amino]benzonitrile

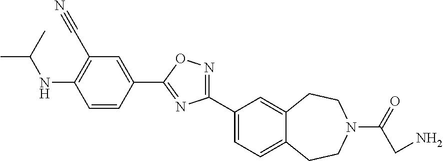

To 1,1-dimethylethyl {2-[7-(5-{3-cyano-4-[(1-methylethyl)amino]phenyl}-1,2,4-oxadiazol-3-yl)-1,2,4,5-tetrahydro-3H-3-benzazepin-3-yl]-2-oxoethyl}carbamate (Preparation 74) (137 mg, 0.26 mmol) in DCM (1.6 ml) was added trifluoroacetic acid (0.4 ml, 5.2 mmol) and the reaction mixture stirred at room temperature for 2 h. The reaction was loaded directly onto an aminopropyl SPE (2 g) and the SPE eluted using methanol in DCM (10%). The appropriate fractions were combined and reduced to dryness under a stream of nitrogen. The residue was dissolved in DMSO (2×1 ml) and purified by MDAP (Method HpH). The solvent was evaporated under a stream of nitrogen to give 5-[3-(3-glycyl-2,3,4,5-tetrahydro-1H-3-benzazepin-7-yl)-1,2,4-oxadiazol-5-yl]-2-[(1-methylethyl)amino]benzonitrile (58 mg).

LCMS (Method formate): Retention time 0.90 min, MH⁺=431

### Example 53

### 5-[3-(3-glycyl-2,3,4,5-tetrahydro-1H-3-benzazepin-7-yl)-1,2,4-oxadiazol-5-yl]-2-(propylamino)benzonitrile

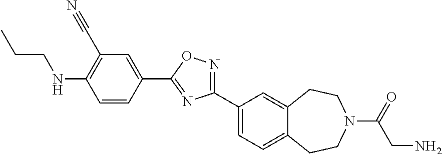

To 1,1-dimethylethyl [2-(7-{5-[3-cyano-4-(propylamino)phenyl}-1,2,4-oxadiazol-3-yl]-1,2,4,5-tetrahydro-3H-3-benzazepin-3-yl)-2-oxoethyl]carbamate (Preparation 75) (110 mg, 0.21 mmol) in DCM (1.6 ml) was added trifluoroacetic acid (0.4 ml, 5.2 mmol) and the reaction mixture stirred at room temperature for 2 h. The reaction was loaded directly onto an aminopropyl SPE (2 g) and the SPE eluted using methanol in DCM (10%). The appropriate fractions were combined and reduced to dryness under a stream of nitrogen. The residue was dissolved in DMSO (2×1 ml) and purified by MDAP (Method HpH). The solvent was evaporated under a stream of nitrogen to give 5-[3-(3-glycyl-2,3,4,5-tetrahydro-1H-3-benzazepin-7-yl)-1,2,4-oxadiazol-5-yl]-2-(propylamino)benzonitrile (52 mg).

LCMS (Method formate): Retention time 0.90 min, MH⁺=431

### Example 54

### 2-{[2-fluoro-1-(fluoromethyl)ethyl]oxy}-5-[3-(3-glycyl-2,3,4,5-tetrahydro-1H-3-benzazepin-7-yl)-1,2,4-oxadiazol-5-yl]benzonitrile

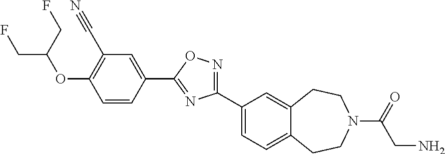

To 1,1-dimethylethyl (2-{7-[5-(3-cyano-4-{[2-fluoro-1-(fluoromethyl)ethyl)oxy}phenyl)-1,2,4-oxadiazol-3-yl]-1,2,4,5-tetrahydro-3H-3-benzazepin-3-yl}-2-oxoethyl)carbamate (Preparation 76) (164 mg, 0.29 mmol) in DCM (1.6 ml) was added trifluoroacetic acid (0.4 ml, 5.2 mmol) and the reaction mixture stirred at room temperature for 2 h. The reaction was loaded directly onto an aminopropyl SPE (2 g) and the SPE eluted using methanol in DCM (10%). The appropriate fractions were combined and reduced to dryness under a stream of nitrogen. The residue was dissolved in DMSO (2×1 ml) and purified by MDAP (Method HpH). The solvent was evaporated under a stream of nitrogen to give 2-{[2-fluoro-1-(fluoromethyl)ethyl)oxy}-5-[3-(3-glycyl-2,3,4,5-tetrahydro-1H-3-benzazepin-7-yl)-1,2,4-oxadiazol-5-yl]benzonitrile (99 mg).

LCMS (Method formate): Retention time 0.81 min, MH⁺=468

### Example 55

### 5-{3-[3-(2,3-dihydroxypropyl)-2,3,4,5-tetrahydro-1H-3-benzazepin-7-yl]-1,2,4-oxadiazol-5-yl}-2-(3-fluoro-1-pyrrolidinyl)benzonitrile

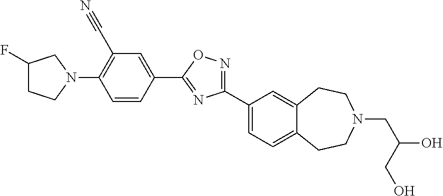

To 2-(3-fluoro-1-pyrrolidinyl)-5-[3-(2,3,4,5-tetrahydro-1H-3-benzazepin-7-yl)-1,2,4-oxadiazol-5-yl]benzonitrile (Preparation 79) (38 mg, 0.094 mmol) in THF (1.5 ml) and DCM (1.5 ml) was added DL-glyceraldehyde (42 mg, 0.47 mmol) and acetic acid (6 μl, 0.099 mmol). The reaction mixture was stirred at room temperature for 10 min. Sodium triacetoxyborohydride (100 mg, 0.47 mmol) was added and the reaction mixture stirred at room temperature overnight. A further portion of sodium triacetoxyborohydride (100 mg, 0.47 mmol) was added and the reaction mixture was stirred at room temperature for 4 h. Further sodium triacetoxyborohydride (100 mg, 0.47 mmol) was added and the reaction was stirred overnight at room temperature. A further portion of sodium triacetoxyborohydride (100 mg, 0.471 mmol) was added and the reaction mixture was stirred for 4 h at room temperature. The reaction mixture was quenched using saturated aqueous sodium hydrogen carbonate solution (30 ml) which was extracted with ethyl acetate (2×50 ml). The organics were combined, dried (hydrophobic frit) and reduced to dryness under a stream of nitrogen. The reaction mixture was dissolved in DMSO (1 ml) and purified by MDAP (Method HpH). The solvent was evaporated under a stream of nitrogen to give 5-{3-[3-(2,3-dihydroxypropyl)-2,3,4,5-tetrahydro-1H-3-benzazepin-7-yl]-1,2,4-oxadiazol-5-yl}-2-(3-fluoro-1-pyrrolidinyl)benzonitrile (8 mg).

LCMS (Method formate): Retention time 0.85 min, MH⁺=448

### Example 56

### 5-[3-(2-D-allothreonyl-5-methyl-1,2,3,4-tetrahydro-6-isoquinolinyl)-1,2,4-oxadiazol-5-yl]-2-[(1-methylethyl)oxy]benzonitrile

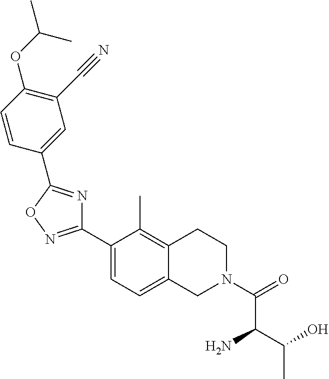

N-{[(1,1-dimethylethyl)oxy]carbonyl}-D-allothreonine (64 mg, 0.29 mmol), N-ethylmorpholine (0.077 ml, 0.61 mmol), HOBT (45 mg, 0.29 mmol) and EDC (56 mg, 0.29 mmol) were dissolved in DMF (2.5 ml), and the mixture was stirred at room temperature for 30 min before adding a solution of 2-[(1-methylethyl)oxy]-5-[3-(5-methyl-1,2,3,4-tetrahydro-6-isoquinolinyl)-1,2,4-oxadiazol-5-yl]benzonitrile hydrochloride (Preparation 25) (100 mg, 0.24 mmol) in DMF (2.5 ml). The reaction mixture was stirred at room temperature for 6 h. The reaction was partitioned between DCM (2×5 ml) and saturated sodium hydrogen carbonate solution (5 ml). The organics were combined, dried (hydrophobic frit) and reduced to dryness in vacuo. The residue was dissolved in DCM (4 ml), and trifluoroacetic acid (1 ml, 13 mmol) was added. The mixture was stirred at room temperature for 2 h, and filtered through an aminopropyl SPE (5 g) washing the SPE with methanol in DCM (10%). The appropriate fractions were combined and evaporated in vacuo, the residue dissolved in DMSO (2×1 ml) and purified by MDAP (x2, Method HpH). The solvent was evaporated under a stream of nitrogen to give 5-[3-(2-D-allothreonyl-5-methyl-1,2,3,4-tetrahydro-6-isoquinolinyl)-1,2,4-oxadiazol-5-yl]-2-[(1-methylethyl)oxy]benzonitrile as a yellow solid (11 mg).

LCMS (Method HpH): Retention time 1.09 min, MH⁺=476

### Example 57

### 2-(3-fluoro-1-pyrrolidinyl)-5-[3-(3-glycyl-2,3,4,5-tetrahydro-1H-3-benzazepin-7-yl)-1,2,4-oxadiazol-5-yl]benzonitrile

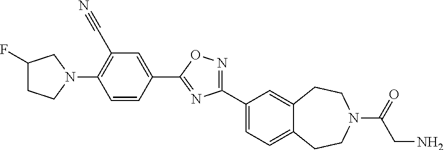

To 1,1-dimethylethyl [2-(7-{5-[3-cyano-4-(3-fluoro-1-pyrrolidinyl)phenyl}-1,2,4-oxadiazol-3-yl]-1,2,4,5-tetrahydro-3H-3-benzazepin-3-yl)-2-oxoethyl]carbamate (Preparation 81) (158 mg, 0.28 mmol) in DCM (1.6 ml) was added trifluoroacetic acid (0.4 ml, 5.2 mmol) and the reaction mixture stirred at room temperature for 2 h. The reaction was applied to an aminopropyl SPE (2 g) and eluted using methanol in DCM (10%). The appropriate fractions were combined and reduced to dryness under a stream of nitrogen. The residue was dissolved in DMSO (1 ml) and purified by MDAP (Method HpH). The solvent was evaporated under a stream of nitrogen to give 2-(3-fluoro-1-pyrrolidinyl)-5-[3-(3-glycyl-2,3,4,5-tetrahydro-1H-3-benzazepin-7-yl)-1,2,4-oxadiazol-5-yl]benzonitrile (13 mg).

LCMS (Method formate): Retention time 0.84 min, MH⁺=461

### Example 58

### 5-[3-(3-D-allothreonyl-2,3,4,5-tetrahydro-1H-3-benzazepin-7-yl)-1,2,4-oxadiazol-5-yl]-2-[(1-methylethyl)oxy]benzonitrile

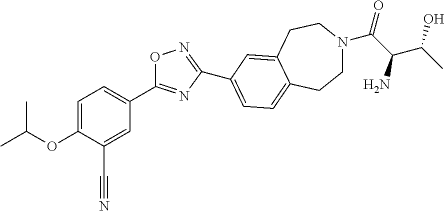

N-{[(1,1-Dimethylethyl)oxy]carbonyl}-D-allothreonine (64 mg, 0.29 mmol), N-ethylmorpholine (0.077 ml, 0.61 mmol), HOBT (45 mg, 0.29 mmol) and EDC (56 mg, 0.29 mmol) were dissolved in DMF (2.5 ml), and the mixture was stirred at room temperature for 30 min before adding a solution of 2-[(1-methylethyl)oxy]-5-[3-(2,3,4,5-tetrahydro-1H-3-benzazepin-7-yl)-1,2,4-oxadiazol-5-yl]benzonitrile hydrochloride (Preparation 43) (100 mg, 0.24 mmol) in DMF (2.5 ml). The reaction mixture was stirred at room temperature overnight, partitioned between DCM (2×5 ml) and saturated sodium hydrogen carbonate solution (1×5 ml). The organic phases were combined, dried (hydrophobic frit) and reduced to dryness in vacuo. The residue was dissolved in DCM (4 ml), and trifluoroacetic acid (1 ml, 13 mmol) was added. The mixture was stirred at room temperature for 2 h, and filtered through an aminopropyl SPE (5 g) washing the SPE with methanol in DCM (10%). The appropriate fractions were combined and evaporated in vacuo, the residue dissolved in DMSO (2×1 ml) and purified by MDAP (x2, Method HpH). The solvent was evaporated under a stream of nitrogen to give 5-[3-(3-D-allothreonyl-2,3,4,5-tetrahydro-1H-3-benzazepin-7-yl)-1,2,4-oxadiazol-5-yl]-2-[(1-methylethyl)oxy]benzonitrile as a white solid (51 mg).

LCMS (Method HpH): Retention time 1.10 min, MH⁺=476

### Example 59

### 2-[(1-methylethyl)oxy]-5-[3-(3-L-threonyl-2,3,4,5-tetrahydro-1H-3-benzazepin-7-yl)-1,2,4-oxadiazol-5-yl]benzonitrile

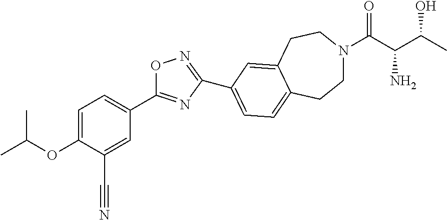

N-{[(1,1-Dimethylethyl)oxy]carbonyl}-L-threonine (64 mg, 0.29 mmol), N-ethylmorpholine (0.077 ml, 0.61 mmol), HOBT (45 mg, 0.29 mmol) and EDC (56 mg, 0.29 mmol) were dissolved in DMF (2.5 ml), and the mixture was stirred at room temperature for 30 min before adding a solution of 2-[(1-methylethyl)oxy]-5-[3-(2,3,4,5-tetrahydro-1H-3-benzazepin-7-yl)-1,2,4-oxadiazol-5-yl]benzonitrile hydrochloride (Preparation 43) (100 mg, 0.24 mmol) in DMF (2.5 ml). The reaction mixture was stirred at room temperature overnight, partitioned between DCM (2×5 ml) and saturated sodium hydrogen carbonate solution (5 ml). The organic phases were combined, dried (hydrophobic frit) and reduced to dryness in vacuo. The residue was dissolved in DCM (4 ml), and trifluoroacetic acid (1 ml, 12.98 mmol) was added. The mixture was stirred at room temperature for 2 h, and filtered through an aminopropyl SPE (5 g), washing the SPE methanol in DCM (10%). The appropriate fractions were combined and evaporated in vacuo, the residue was dissolved in DMSO (2×1 ml) and purified by MDAP (x2, Method HpH). The solvent was evaporated under a stream of nitrogen to 2-[(1-methylethyl)oxy]-5-[3-(3-L-threonyl-2,3,4,5-tetrahydro-1H-3-benzazepin-7-yl)-1,2,4-oxadiazol-5-yl]benzonitrile as a white solid (51 mg).

LCMS (Method HpH): Retention time 1.10 min, MH⁺=476

### Example 60

### 5-{3-[3-(2,3-dihydroxypropyl)-2,3,4,5-tetrahydro-1H-3-benzazepin-7-yl]-1,2,4-oxadiazol-5-yl}-2-{[2-fluoro-1-(fluoromethyl)ethyl]oxy}benzonitrile

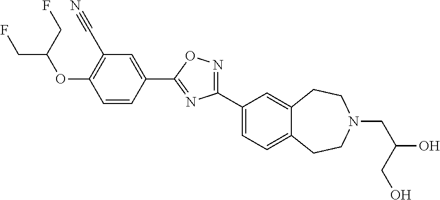

To 2-{[2-fluoro-1-(fluoromethyl)ethyl)oxy}-5-[3-(2,3,4,5-tetrahydro-1H-3-benzazepin-7-yl)-1,2,4-oxadiazol-5-yl]benzonitrile (Preparation 77) (152 mg, 0.37 mmol) in THF (1.5 ml) and DCM) (1.5 ml) was added DL-glyceraldehyde (167 mg, 1.9 mmol) and acetic acid (0.022 ml, 0.39 mmol) and the reaction mixture stirred at room temperature for 10 min. Sodium triacetoxyborohydride (392 mg, 1.9 mmol) was added and the reaction mixture stirred at room temperature overnight. A further portion of sodium triacetoxyborohydride (392 mg, 1.9 mmol) was added and the reaction mixture stirred at room temperature for 4 h. The reaction mixture was quenched using saturated aqueous sodium hydrogen carbonate solution (30 ml), which was extracted with ethyl acetate (2×50 ml). The organics were combined, dried (hydrophobic frit) and reduced to dryness under a stream of nitrogen. The residue was dissolved in DMSO (2×1 ml) and purified by MDAP (x2, Method HpH). The solvent was evaporated under a stream of nitrogen to give 5-{3-[3-(2,3-dihydroxypropyl)-2,3,4,5-tetrahydro-1H-3-benzazepin-7-yl]-1,2,4-oxadiazol-5-yl}-2-{[2-fluoro-1-(fluoromethyl)ethyl)oxy}benzonitrile (63 mg).

LCMS (Method formate): Retention time 0.77 min, MH⁺=485

### Example 61

### 2-(ethyloxy)-5-[3-(3-glycyl-2,3,4,5-tetrahydro-1H-3-benzazepin-7-yl)-1,2,4-oxadiazol-5-yl]benzonitrile

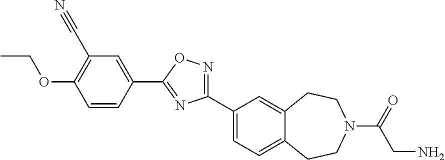

To 1,1-dimethylethyl [2-(7-{5-[3-cyano-4-(ethyloxy)phenyl}-1,2,4-oxadiazol-3-yl]-1,2,4,5-tetrahydro-3H-3-benzazepin-3-yl)-2-oxoethyl]carbamate (Preparation 82) (259 mg, 0.50 mmol) in DCM (1.6 ml) was added trifluoroacetic acid (0.4 ml, 5.2 mmol) and the reaction mixture stirred at room temperature for 2 h. The reaction was applied to an aminopropyl SPE (2 g) and the SPE eluted using methanol in DCM (10%). The appropriate fractions were combined and reduced to dryness under a stream of nitrogen to give 2-(ethyloxy)-5-[3-(3-glycyl-2,3,4,5-tetrahydro-1H-3-benzazepin-7-yl)-1,2,4-oxadiazol-5-yl]benzonitrile (78 mg).

LCMS (Method formate): Retention time 0.84 min, MH⁺=418

### Example 62

### 5-[3-(3-glycyl-2,3,4,5-tetrahydro-1H-3-benzazepin-7-yl)-1,2,4-oxadiazol-5-yl]-2-(propyloxy)benzonitrile

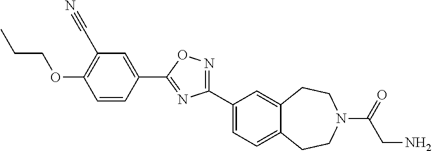

To 1,1-dimethylethyl [2-(7-{5-[3-cyano-4-(propyloxy)phenyl]-1,2,4-oxadiazol-3-yl}-1,2,4,5-tetrahydro-3H-3-benzazepin-3-yl)-2-oxoethyl]carbamate (Preparation 85) (81 mg, 0.15 mmol) in DCM (1.6 ml) was added trifluoroacetic acid (0.4 ml, 5.2 mmol) and the reaction mixture was stirred at room temperature for 2 h. The reaction was applied to an aminopropyl SPE (2 g) and the SPE eluted using methanol in DCM (10%). The appropriate fractions were combined and reduced to dryness under a stream of nitrogen. The residue was dissolved in DMSO (2×1 ml) and purified by MDAP (x2, Method HpH). The solvent was evaporated under a stream of nitrogen to give 5-[3-(3-glycyl-2,3,4,5-tetrahydro-1H-3-benzazepin-7-yl)-1,2,4-oxadiazol-5-yl]-2-(propyloxy)benzonitrile (65 mg).

LCMS (Method formate): Retention time 0.91 min, MH⁺=432

### Example 63

### 5-{3-[3-(4-aminobutanoyl)-2,3,4,5-tetrahydro-1H-3-benzazepin-7-yl]-1,2,4-oxadiazol-5-yl}-2-[(1-methylethyl)oxy]benzonitrile

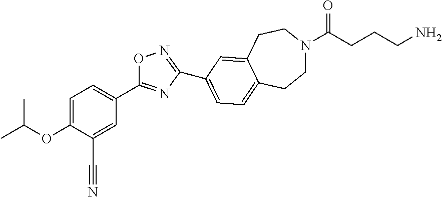

4-({[(1,1-Dimethylethyl)oxy]carbonyl}amino)butanoic acid (59 mg, 0.29 mmol), N-ethylmorpholine (0.077 ml, 0.61 mmol), HOBT (45 mg, 0.29 mmol) and EDC (56 mg, 0.29 mmol) were dissolved in DMF (2.0 ml), and the mixture was stirred at room temperature for 30 min before adding a solution of 2-[(1-methylethyl)oxy]-5-[3-(2,3,4,5-tetrahydro-1H-3-benzazepin-7-yl)-1,2,4-oxadiazol-5-yl]benzonitrile hydrochloride (Preparation 43) (100 mg, 0.24 mmol) in DMF (10 ml). The reaction mixture was stirred at room temperature overnight, partitioned between DCM (2×5 ml) and saturated sodium hydrogen carbonate solution (5 ml). The organic phases were combined, dried (hydrophobic frit) and reduced to dryness in vacuo. The residue was dissolved in DCM (4 ml), and trifluoroacetic acid (1 ml, 13 mmol) was added. The mixture was stirred at room temperature for 2 h, filtered through an aminopropyl SPE (5 g) washing the SPE with methanol in DCM (10%). The appropriate fractions were combined and evaporated in vacuo, the residue was dissolved in DMSO (2×1 ml) and purified by MDAP (x2, Method HpH). The solvent was evaporated under a stream of nitrogen to give 5-{3-[3-(4-aminobutanoyl)-2,3,4,5-tetrahydro-1H-3-benzazepin-7-yl]-1,2,4-oxadiazol-5-yl}-2-[(1-methylethyl)oxy]benzonitrile (65 mg).

LCMS (Method HpH): Retention time 1.13 min, MH⁺=460

### Example 64

### 5-(3-{2-[2-hydroxy-1-(hydroxymethyl)ethyl]-5-methyl-1,2,3,4-tetrahydro-6-isoquinolinyl}-1,2,4-oxadiazol-5-yl)-2-[(1-methylethyl)oxy]benzonitrile

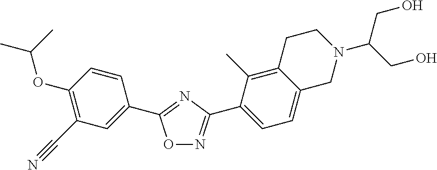

To a stirred solution of the impure 5-{3-[2-(2,2-dimethyl-1,3-dioxan-5-yl)-5-methyl-1,2,3,4-tetrahydro-6-isoquinolinyl]-1,2,4-oxadiazol-5-yl}-2-[(1-methylethyl)oxy]benzonitrile (Preparation 86) (277 mg, max. 0.45 mmol) in THF (6 ml) was added hydrochloric acid (3M, 6 ml, 18 mmol). The mixture was stirred at room temperature for 16 h. The volatiles were evaporated under a stream of nitrogen and the residue was partitioned between saturated aqueous sodium hydrogen carbonate solution (5 ml) and ethyl acetate (5 ml). The phases were separated and the aqueous phase extracted with ethyl acetate (3×4 ml). The aqueous phase was evaporated to dryness under a stream of nitrogen and the residue was triturated with DCM (4×4 ml). The combined ethyl acetate and DCM organic phases were dried (hydrophobic frit) and were evaporated to dryness under a stream of nitrogen. The residue was purified by flash chromatography on a silica cartridge (20 g), eluting with a methanol/DCM gradient (0-20% methanol). The required fractions were combined and evaporated under a stream of nitrogen to give a residue which was further purified by MDAP (Method formate). The required fraction was evaporated under a stream of nitrogen to give 5-(3-{2-[2-hydroxy-1-(hydroxymethyl)ethyl]-5-methyl-1,2,3,4-tetrahydro-6-isoquinolinyl}-1,2,4-oxadiazol-5-yl)-2-[(1-methylethyl)oxy]benzonitrile as a white crunchy foam (127 mg, 63%)

LCMS (Method formate): Retention time 0.88 min, MH⁺=449

### Example 65

### 5-(3-{2-[2-hydroxy-1-(hydroxymethyl)ethyl]-5-methyl-1,2,3,4-tetrahydro-6-isoquinolinyl}-1,2,4-oxadiazol-5-yl)-2-[(1-methylethyl)oxy]benzonitrile

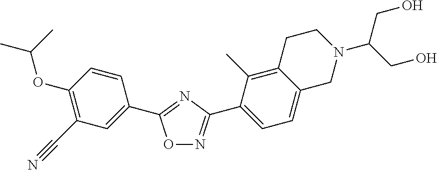

A mixture of 5-{3-[2-(2,2-dimethyl-1,3-dioxan-5-yl)-5-methyl-1,2,3,4-tetrahydro-6-isoquinolinyl]-1,2,4-oxadiazol-5-yl}-2-[(1-methylethyl)oxy]benzonitrile (Preparation 86) (35.8 g, 73 mmol) tetrahydrofuran (50 ml) and hydrochloric acid (2M, 50 ml) was allowed to stand at room temperature overnight. The THF was evaporated. The residue was basified to pH10 by portionwise addition of sodium hydroxide (2M). The mixture was stirred for 20 min at room temperature, the precipitated solid collected by filtration and washed with water (100 ml). The wet solid was partially dried at 40° C. in vacuo and suspended in ethanol (600 ml). The suspension was stirred and heated to boiling point, giving a clear pale yellow solution. This was allowed to cool slowly to room temperature, placed in an ice bath and cooled to 5° C. over 30 min. The solid was collected by filtration and washed with ethanol (100 ml), then dried in vacuo overnight at 45° C. to give 5-(3-{2-[2-hydroxy-1-(hydroxymethyl)ethyl]-5-methyl-1,2,3,4-tetrahydro-6-isoquinolinyl}-1,2,4-oxadiazol-5-yl)-2-[(1-methylethyl)oxy]benzonitrile (30.7 g, 93%) as pale cream crystalline solid.

LCMS (Method HpH): Retention time 1.11 min, MH⁺=449

1H NMR (D₆-DMSO): δ H 8.47 (1H, d), 8.37 (1H, dd), 7.63 (1H, d) 7.54 (1H, d), 7.07 (1H, d), 4.96 (1H, m), 4.38 (2H, m), 3.91 (2H, s), 3.64-3.53 (4H, m), 2.96 (2H, t), 2.74-2.66 (3H, m), 2.41 (3H, s), 1.38 (6H, d).

### Example 66

### 5-(3-{2-[2-hydroxy-1-(hydroxymethyl)ethyl]-5-methyl-1,2,3,4-tetrahydro-6-isoquinolinyl}-1,2,4-oxadiazol-5-yl)-2-[(1-methylethyl)oxy]benzonitrile hydrochloride

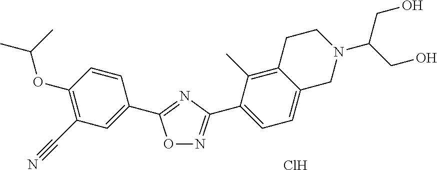

5-(3-{2-[2-hydroxy-1-(hydroxymethyl)ethyl]-5-methyl-1,2,3,4-tetrahydro-6-isoquinolinyl}-1,2,4-oxadiazol-5-yl)-2-[(1-methylethyl)oxy]benzonitrile (Example 2) (15.1 g) was suspended in DCM (200 ml) and hydrogen chloride in isopropanol (˜4M, 12 ml) was added drop-wise and the reaction stirred for 30 min. Ether (300 ml) was added and the resulting paste was filtered. The colourless solid was washed with diethyl ether (100 ml) and dried at 45° C. over the weekend to give 5-(3-{2-[2-hydroxy-1-(hydroxymethyl)ethyl]-5-methyl-1,2,3,4-tetrahydro-6-isoquinolinyl}-1,2,4-oxadiazol-5-yl)-2-[(1-methylethyl)oxy]benzonitrile hydrochloride (16.0 g, 45%) as a white solid.

LCMS (Method HpH): Retention time 1.12 min, MH⁺=449

1H NMR (D₆-DMSO): δ H 10.58 (1H, bs), 8.49 (1H, d), 8.39 (1H, dd), 7.77 (1H, d), 7.56 (1H, d), 7.27 (1H, d), 5.53 (2H, bs), 4.97 (1H, m), 4.78-4.72 (1H, m), 4.60-4.56 (1H, m), 3.90 (5H, m), 3.56 (1H, m), 3.40 (1H, partially obscured by water), 3.22-3.08 (2H, m), 2.46 (3H, s), 1.38 (6H, d).

### Example 67

### 5-{3-[3-(N-methyl-b-alanyl)-2,3,4,5-tetrahydro-1H-3-benzazepin-7-yl]-1,2,4-oxadiazol-5-yl}-2-[(1-methylethyl)oxy]benzonitrile

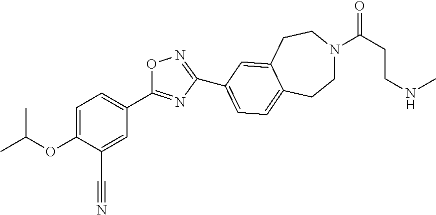

N-{[(1,1-Dimethylethyl)oxy]carbonyl}-N-methyl-b-alanine (59 mg, 0.29 mmol), N-ethylmorpholine (0.077 ml, 0.61 mmol), HOBT (45 mg, 0.292 mmol) and EDC (56 mg, 0.29 mmol) were dissolved in DMF (2.0 ml), and the mixture was stirred at room temperature for 30 min before adding a solution of 2-[(1-methylethyl)oxy]-5-[3-(2,3,4,5-tetrahydro-1H-3-benzazepin-7-yl)-1,2,4-oxadiazol-5-yl]benzonitrile hydrochloride (Preparation 43) (100 mg, 0.24 mmol) in DMF (10 ml). The reaction mixture was stirred at room temperature overnight, partitioned between DCM (2×5 ml) and saturated sodium hydrogen carbonate solution (5 ml). The organic phases were combined, dried (hydrophobic frit) and evaporated in vacuo. The residue was dissolved in DCM (4 ml) and trifluoroacetic acid (1 ml, 12.98 mmol) was added. The mixture was stirred at room temperature for 2 h and filtered through an aminopropyl SPE (5 g), washing with methanol in DCM (10%). The appropriate fractions were combined and evaporated in vacuo, the residue was dissolved in DMSO (2×1 ml) and purified by MDAP (Method HpH). The solvent was evaporated under a stream of nitrogen to give 5-{3-[3-(N-methyl-b-alanyl)-2,3,4,5-tetrahydro-1H-3-benzazepin-7-yl]-1,2,4-oxadiazol-5-yl}-2-[(1-methylethyl)oxy]benzonitrile as a yellow gum (66 mg).

LCMS (Method HpH): Retention time 1.16 min, MH⁺=460

### Example 68

### 5-(3-{3-[2-hydroxy-1-(hydroxymethyl)ethyl]-2,3,4,5-tetrahydro-1H-3-benzazepin-7-yl}-1,2,4-oxadiazol-5-yl)-2-[(1-methylethyl)oxy]benzonitrile

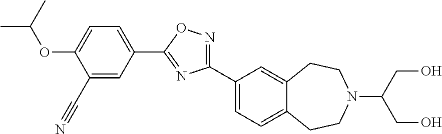

To a stirred solution the impure 5-{3-[3-(2,2-dimethyl-1,3-dioxan-5-yl)-2,3,4,5-tetrahydro-1H-3-benzazepin-7-yl]-1,2,4-oxadiazol-5-yl}-2-[(1-methylethyl)oxy]benzonitrile (Preparation 87) (286 mg, max. 0.44 mmol) in THF (6 ml) was added hydrochloric acid (2M, 6 ml, 18 mmol). The mixture was stirred at room temperature for 16 h. The volatiles were evaporated under a stream of nitrogen and the residue was partitioned between saturated aqueous sodium hydrogen carbonate solution (5 ml) and ethyl acetate (5 ml). The phases were separated and the aqueous phase extracted with ethyl acetate (3×4 ml). The combined organic phases were dried (hydrophobic frit) and were evaporated to dryness under a stream of nitrogen. The residue was purified by flash chromatography on a silica cartridge (20 g), eluting with a methanol/DCM gradient (0-20% methanol). The required fractions were combined and evaporated under a stream of nitrogen to give a residue which was further purified by MDAP (Method formate). The required fraction was evaporated under a stream of nitrogen to give 5-(3-{3-[2-hydroxy-1-(hydroxymethyl)ethyl]-2,3,4,5-tetrahydro-1H-3-benzazepin-7-yl}-1,2,4-oxadiazol-5-yl)-2-[(1-methylethyl)oxy]benzonitrile as a cream coloured crunchy foam (66 mg, 34%).

LCMS (Method formate): Retention time 0.88 min, MH⁺=449

### Example 69

### 5-[3-(3-β-alanyl-2,3,4,5-tetrahydro-1H-3-benzazepin-7-yl)-1,2,4-oxadiazol-5-yl]-2-[(1-methylethyl)oxy]benzonitrile

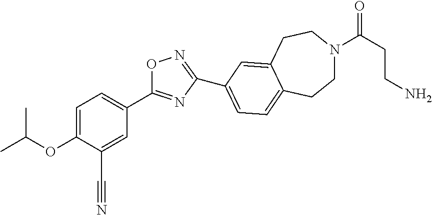

N-{[(1,1-Dimethylethyl)oxy]carbonyl}-b-alanine (55 mg, 0.29 mmol), N-ethylmorpholine (0.077 ml, 0.61 mmol), HOBT (45 mg, 0.29 mmol) and EDC (56 mg, 0.29 mmol) were dissolved in DMF (2.0 ml), and the mixture was stirred at room temperature for 30 min before adding a solution of 2-[(1-methylethyl)oxy]-5-[3-(2,3,4,5-tetrahydro-1H-3-benzazepin-7-yl)-1,2,4-oxadiazol-5-yl]benzonitrile hydrochloride (Preparation 43) (100 mg, 0.24 mmol) in DMF (10 ml). The reaction mixture was stirred at room temperature overnight, partitioned between DCM (2×5 ml) and saturated sodium hydrogen carbonate solution (5 ml). The organic phases were combined, dried (hydrophobic frit) and evaporated in vacuo. The residue was dissolved in DCM (4 ml) and trifluoroacetic acid (1 ml, 12.98 mmol) was added. The mixture was stirred at room temperature for 2 h, and filtered through an aminopropyl SPE (5 g), washing with methanol in DCM (10%). The appropriate fractions were combined and reduced to dryness in vacuo, the residue dissolved in DMSO (2×1 ml) and purified by MDAP (Method HpH). The solvent was evaporated under a stream of nitrogen to give 5-[3-(3-β-alanyl-2,3,4,5-tetrahydro-1H-3-benzazepin-7-yl)-1,2,4-oxadiazol-5-yl]-2-[(1-methylethyl)oxy]benzonitrile as a white solid (72 mg).

LCMS (Method HpH): Retention time 1.12 min, MH⁺=446

### Example 70

### 5-{3-[3-(2,3-dihydroxypropyl)-2,3,4,5-tetrahydro-1H-3-benzazepin-7-yl]-1,2,4-oxadiazol-5-yl}-2-(ethyloxy)benzonitrile

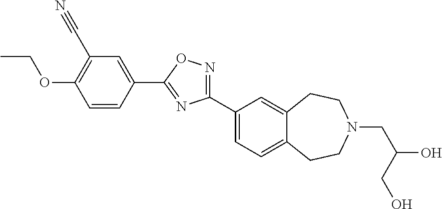

To 2-(ethyloxy)-5-[3-(2,3,4,5-tetrahydro-1H-3-benzazepin-7-yl)-1,2,4-oxadiazol-5-yl]benzonitrile (Preparation 83) (82 mg, 0.23 mmol) in THF (1.5 ml) and DCM (1.5 ml) was added DL-glyceraldehyde (102 mg, 1.1 mmol) and acetic acid (0.014 ml, 0.24 mmol) and the reaction mixture stirred at room temperature for 10 min. Sodium triacetoxyborohydride (241 mg, 1.1 mmol) was added and the reaction mixture stirred at room temperature overnight. Further sodium triacetoxyborohydride (241 mg, 1.1 mmol) was added and the reaction mixture stirred at room temperature for 4 h. The reaction mixture was quenched by addition of saturated aqueous sodium hydrogen carbonate solution (30 ml), which was extracted with ethyl acetate (2×50 ml). The organics were combined, dried (hydrophobic frit) and reduced to dryness under a stream of nitrogen. DMSO (2 ml) was added to the sample and evaporated under a stream of nitrogen. The residue was dissolved in DCM and applied to a silica cartridge (40 g) which was eluted with an 2M ammonia in methanol/DCM gradient (0-8% 2M ammonia in methanol) followed by further elution with 2M ammonia in methanol/DCM (8%). The appropriate fractions were combined and evaporated in vacuo. The sample was dissolved in DMSO (1 ml) and purified by MDAP (Method HpH). The solvent was evaporated under a stream of nitrogen to give 5-{3-[3-(2,3-dihydroxypropyl)-2,3,4,5-tetrahydro-1H-3-benzazepin-7-yl]-1,2,4-oxadiazol-5-yl}-2-(ethyloxy)benzonitrile (40 mg).

LCMS (Method HpH): Retention time 1.10 min, MH⁺=435

### Example 71

### 5-(3-{2-[(2S)-2,3-dihydroxypropyl]-5-methyl-1,2,3,4-tetrahydro-6-isoquinolinyl}-1,2,4-oxadiazol-5-yl)-2-[(1-methylethyl)oxy]benzonitrile hydrochloride

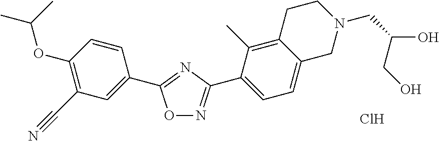

Sodium triacetoxyborohydride (2.17 g, 10 mmol) was added portionwise to a stirred solution of 2-[(1-methylethyl)oxy]-5-[3-(1,2,3,4-tetrahydro-6-isoquinolinyl)-1,2,4-oxadiazol-5-yl]benzonitrile trifluororacetate (Preparation 25) (1 g, 2.1 mmol) and D-glyceraldehyde (922 mg, 10 mmol) in DCM (50 ml) and methanol (10 ml). After complete addition the reaction mixture was stirred at room temperature overnight. Saturated sodium hydrogen carbonate (30 ml) was added and the reaction mixture and stirred for 15 min. The organic phase was separated and the aqueous phase extracted with DCM (2×15 ml). The combined organics were dried and evaporated. The residue was chromatographed (methanol/DCM, 0-6%). The combined product fractions were treated with hydrogen chloride in diethyl ether (1M, 3 ml). The solvent was evaporated and the residue triturated with diethyl ether to give 5-(3-{2-[(2S)-2,3-dihydroxypropyl]-5-methyl-1,2,3,4-tetrahydro-6-isoquinolinyl}-1,2,4-oxadiazol-5-yl)-2-[(1-methylethyl)oxy]benzonitrile hydrochloride as a colourless solid (654 mg).

LCMS (Method formate): Retention time 0.87 min, MH⁺=449

### Example 72

### 5-(3-{3-[(2S)-2,3-dihydroxypropyl]-2,3,4,5-tetrahydro-1H-3-benzazepin-7-yl}-1,2,4-oxadiazol-5-yl)-2-[(1-methylethyl)oxy]benzonitrile

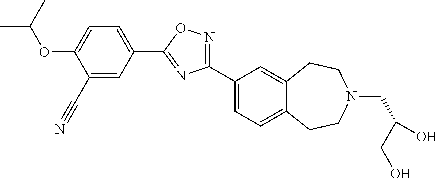

To a stirred suspension of D-(+)-glyceraldehyde (127 mg, 1.4 mmol) and 2-[(1-methylethyl)oxy]-5-[3-(2,3,4,5-tetrahydro-1H-3-benzazepin-7-yl)-1,2,4-oxadiazol-5-yl]benzonitrile hydrochloride (Preparation 43) (187 mg, 0.46 mmol) in DCM (5 ml) under nitrogen, was added sodium triacetoxyborohydride (436 mg, 2.1 mmol) in two portions. The mixture was stirred at room temperature for 16 h. To the mixture was added acetic acid (0.039 ml, 0.68 mmol) followed by further D-(+)-glyceraldehyde (127 mg, 1.4 mmol) and after 30 min further sodium triacetoxyborohydride (109 mg, 0.5 mmol) and DCM (5 ml). Stirring at room temperature was continued for a further 23 h. Further sodium triacetoxyborohydride (100 mg, 0.47 mmol) was added and stirring continued for a further 6 h. Saturated aqueous sodium hydrogen carbonate solution (5 ml) was added to the mixture which was then stirred vigorously for 15 min. The mixture was evaporated to dryness under a stream of nitrogen and the residue was partitioned between saturated aqueous sodium hydrogen carbonate solution (5 ml) and ethyl acetate (5 ml). The phases were separated and the aqueous phase extracted with ethyl acetate (3×4 ml). The combined organic phases were dried (hydrophobic frit) and evaporated to dryness under a stream of nitrogen. The residue was purified by flash chromatography on a silica cartridge (20 g) eluting with a methanol/DCM gradient (0-20%). The required fractions were combined and evaporated under a stream of nitrogen to give a residue which was further purified by MDAP (Method formate). The required fraction was evaporated under a stream of nitrogen to give 5-(3-{3-[(2S)-2,3-dihydroxypropyl]-2,3,4,5-tetrahydro-1H-3-benzazepin-7-yl}-1,2,4-oxadiazol-5-yl)-2-[(1-methylethyl)oxy]benzonitrile as a white crunchy foam, (94 mg, 46%)

LCMS (Method formate): Retention time 0.87 min, MH⁺=449

### Example 73

### 5-(3-{2-[(2R)-2,3-dihydroxypropyl]-5-methyl-1,2,3,4-tetrahydro-6-isoquinolinyl}-1,2,4-oxadiazol-5-yl)-2-[(1-methylethyl)oxy]benzonitrile hydrochloride

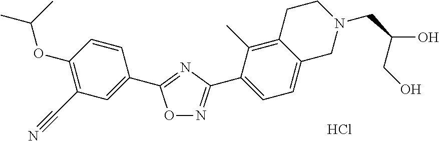

L-glyceraldehyde (1.84 g, 21 mmol) was added to a stirred solution of 2-[(1-methylethyl)oxy]-5-[3-(5-methyl-1,2,3,4-tetrahydro-6-isoquinolinyl)-1,2,4-oxadiazol-5-yl]benzonitrile trifluoroacetate (Preparation 25) (2.0 g, 4.1 mmol) in DCM (50 ml) and methanol (5 ml). The mixture was stirred at room temperature for 20 min then treated with sodium triacetoxyborohydride (4.34 g, 21 mmol). The reaction mixture was stirred at room temperature overnight. The reaction mixture was concentrated to approx. half volume and diluted with ethyl acetate (50 ml). The mixture was washed with saturated sodium hydrogen carbonate (3×25 ml). The organic phase was separated, washed with brine, dried and evaporated. The residue was chromatographed (methanol/DCM, 5%). The product was dissolved in DCM (20 ml) and treated with hydrogen chloride in diethyl ether (1M, 5 ml). The solvent was evaporated and the residue triturated with diethyl ether to give 5-(3-{2-[(2R)-2,3-dihydroxypropyl]-5-methyl-1,2,3,4-tetrahydro-6-isoquinolinyl}-1,2,4-oxadiazol-5-yl)-2-[(1-methylethyl)oxy]benzonitrile hydrochloride as a colourless solid (1.0 g).

LCMS (Method formate): Retention time 0.84 min, MH⁺=449

### Example 74

### 5-(3-{3-[(2R)-2,3-dihydroxypropyl]-2,3,4,5-tetrahydro-1H-3-benzazepin-7-yl}-1,2,4-oxadiazol-5-yl)-2-[(1-methylethyl)oxy]benzonitrile

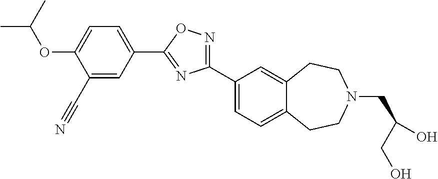

To a stirred suspension of L-(−)-glyceraldehyde (127 mg, 1.4 mmol) and 2-[(1-methylethyl)oxy]-5-[3-(2,3,4,5-tetrahydro-1H-3-benzazepin-7-yl)-1,2,4-oxadiazol-5-yl]benzonitrile hydrochloride (Preparation 43) (187 mg, 0.46 mmol) in DCM (10 ml) under nitrogen, was added acetic acid (0.039 ml, 0.68 mmol) followed by sodium triacetoxyborohydride (436 mg, 2.1 mmol). The mixture was stirred at room temperature for 20 h. Further sodium triacetoxyborohydride (193 mg, 0.91 mmol) was added and stirring continued for 72 h. Saturated aqueous sodium hydrogen carbonate solution (5 ml) was added and the mixture stirred vigorously for 30 min. The mixture was evaporated to dryness under a stream of nitrogen and the residue was partitioned between saturated aqueous sodium hydrogen carbonate solution (5 ml) and ethyl acetate (5 ml). The phases were separated and the aqueous phase extracted with ethyl acetate (3×4 ml). The combined organic phases were dried (hydrophobic frit) and evaporated to dryness under a stream of nitrogen. The residue was purified by MDAP (Method formate). The required fraction was evaporated under a stream of nitrogen to give 5-(3-{3-[(2R)-2,3-dihydroxypropyl]-2,3,4,5-tetrahydro-1H-3-benzazepin-7-yl}-1,2,4-oxadiazol-5-yl)-2-[(1-methylethyl)oxy]benzonitrile as a white crunchy foam (140 mg, 69%).

LCMS (Method formate): Retention time 0.87 min, MH⁺=449

### Example 75

### 2-[7-(5-{3-cyano-4-[(1-methylethyl)oxy]phenyl}-1,2,4-oxadiazol-3-yl)-1,2,4,5-tetrahydro-3H-3-benzazepin-3-yl}-N-[(2S)-2-hydroxypropyl]acetamide hydrochloride

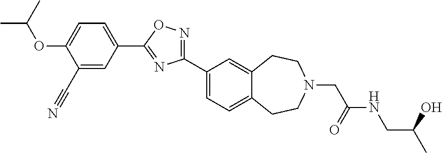

To a solution of 2-[7-(5-{3-cyano-4-[(1-methylethyl)oxy]phenyl}-1,2,4-oxadiazol-3-yl)-1,2,4,5-tetrahydro-3H-3-benzazepin-3-yl]-N-((2S)-2-{[(1,1-dimethylethyl)(dimethyl)silyl]oxy}propyl)acetamide (Preparation 88) (121 mg, 0.2 mmol) in THF (2 ml) at room temperature was added tetrabutylammonium fluoride (TBAF, 1N in THF) (105 mg, 0.40 mmol) and the resulting mixture was stirred at this temperature for 1 h 10 min. The solvent was evaporated, the residue diluted with ethyl acetate and the mixture washed with saturated sodium hydrogen carbonate (x2). The organic phase was dried (MgSO₄) and concentrated in vacuo. The residue was dissolved in 1,4-dioxane (2 ml) and treated with hydrogen chloride in 1,4-dioxane (4N, 1 ml). The solvent was evaporated and the residue triturated with diethyl ether to give a precipitate which was isolated by filtration to give 2-[7-(5-{3-cyano-4-[(1-methylethyl)oxy]phenyl}-1,2,4-oxadiazol-3-yl)-1,2,4,5-tetrahydro-3H-3-benzazepin-3-yl]-N-[(2S)-2-hydroxypropyl]acetamide hydrochloride as a white solid (42 mg, 40%)

LCMS (Method HpH): Retention time 1.19 min, MH⁺=490

### Example 76

### 5-{3-[2-(2,3-dihydroxypropyl)-1,2,3,4-tetrahydro-5-isoquinolinyl]-1,2,4-oxadiazol-5-yl}-2-[(1-methylethyl)oxy]benzonitrile

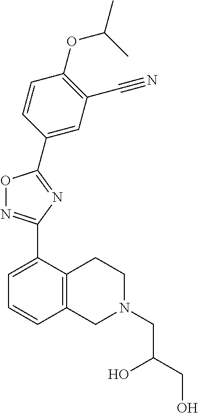

To a stirred suspension of 2-[(1-methylethyl)oxy]-5-[3-(1,2,3,4-tetrahydro-5-isoquinolinyl)-1,2,4-oxadiazol-5-yl]benzonitrile trifluoroacetate (Preparation 91) (0.474 g, 1 mmol) in DCM (5 ml) and THF (5 ml) was added DL-glyceraldehyde (0.45 g, 5 mmol) and acetic acid (0.063 ml, 1.1 mmol). The reaction was stirred at room temperature for 10 min. Sodium triacetoxyborohydride (1.06 g, 5 mmol) was added and the reaction stirred at room temperature. After stirring for ˜24 h, DL-glyceraldehyde (0.45 g, 5 mmol), acetic acid (0.063 ml, 1.1 mmol) and sodium triacetoxyborohydride (1.06 g, 5 mmol) were added to the reaction mixture. The solution was stirred for 3 days and saturated sodium hydrogen carbonate (5 ml) was slowly added to the mixture. The reaction mixture was partitioned between water (30 ml) and ethyl acetate (3×30 ml). The combined organic phases were dried (hydrophobic frit) and concentrated under reduced pressure. The resultant oil was dissolved in DCM and loaded onto a silica cartridge (25 g) and the cartridge eluted with a methanol/DCM gradient (0-5%). The appropriate fractions were combined and evaporated under vacuum to give 5-{3-[2-(2,3-dihydroxypropyl)-1,2,3,4-tetrahydro-5-isoquinolinyl]-1,2,4-oxadiazol-5-yl}-2-[(1-methylethyl)oxy]benzonitrile (89 mg, 20%) as a yellow solid.

LCMS (Method formate): Retention time 0.82 min, MH⁺=435

### Example 77

### 2-[(1-methylethyl)oxy]-5-[3-(3-L-threonyl-2,3,4,5-tetrahydro-1H-3-benzazepin-7-yl)-1,2,4-oxadiazol-5-yl]-3-pyridinecarbonitrile hydrochloride

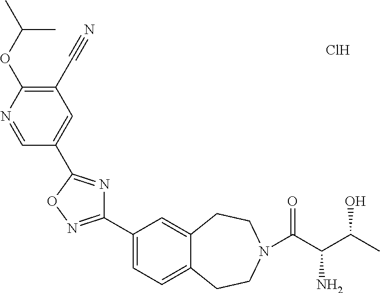

Hydrogen chloride in 1,4-dioxane (4.0M, 0.126 ml, 0.50 mmol) was added dropwise to a solution of 1,1-dimethylethyl ((1S,2R)-1-{[7-(5-{5-cyano-6-[(1-methylethyl)oxy]-3-pyridinyl}-1,2,4-oxadiazol-3-yl)-1,2,4,5-tetrahydro-3H-3-benzazepin-3-yl}carbonyl]-2-hydroxypropyl)carbamate (Preparation 95) (58 mg, 0.10 mmol) in dry DCM (0.5 ml) at 0° C. and the mixture stirred for 16 h. The solvent was evaporated and the residual solid triturated under diethyl ether (1 ml). The solvent was decanted and the reside dried under vacuum to give 2-[(1-methylethyl)oxy]-5-[3-(3-L-threonyl-2,3,4,5-tetrahydro-1H-3-benzazepin-7-yl)-1,2,4-oxadiazol-5-yl]-3-pyridinecarbonitrile hydrochloride (50 mg, 87%).

LCMS (Method formate): Retention time 0.94 min, MH⁺=477

### Example 78

### 2-[7-(5-{3-cyano-4-[(1-methylethyl)oxy]phenyl}-1,2,4-oxadiazol-3-yl)-1,2,4,5-tetrahydro-3H-3-benzazepin-3-yl]-N-(1R)-2-hydroxy-1-methylethyl]acetamide hydrochloride

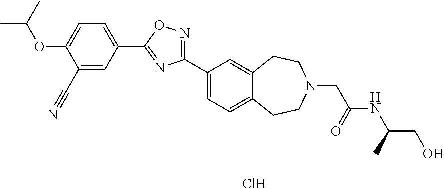

A solution of 2-[7-(5-{3-cyano-4-[(1-methylethyl)oxy]phenyl}-1,2,4-oxadiazol-3-yl)-1,2,4,5-tetrahydro-3H-3-benzazepin-3-yl]-N-((1R)-2-{[(1,1-dimethylethyl)(dimethyl)silyl]oxy}-1-methylethyl)acetamide (Preparation 99) (121 mg, 0.2 mmol) in THF (2 ml) at room temperature was treated with tetrabutylammonium fluoride (1N in THF, 0.4 ml, 0.40 mmol) and the resulting pale yellow mixture was stirred at room temperature for 1.5 h. The solvent was evaporated and the residue diluted with ethyl acetate. The mixture was washed with saturated sodium hydrogen carbonate (x2) dried (MgSO₄) and concentrated in vacuo. The residue was dissolved in 1,4-dioxane (2 ml) and treated with hydrogen chloride in 1,4-dioxane (4N, 1 ml). Removal of the solvent and trituration with diethyl ether gave a precipitate which was isolated by filtration to give 2-[7-(5-{3-cyano-4-[(1-methylethyl)oxy]phenyl}-1,2,4-oxadiazol-3-yl)-1,2,4,5-tetrahydro-3H-3-benzazepin-3-yl]-N-(1R)-2-hydroxy-1-methylethyl]acetamide hydrochloride as a white solid (71 mg, 68%).

LCMS (Method HpH): Retention time 1.20 min, MH⁺=490

### Example 79

### 2-[(1-methylethyl)oxy]-5-[3-(5-methyl-2-L-threonyl-1,2,3,4-tetrahydro-6-isoquinolinyl)-1,2,4-oxadiazol-5-yl]-3-pyridinecarbonitrile hydrochloride

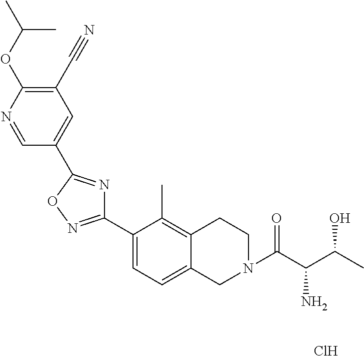

Hydrogen chloride in 1,4-dioxane (4M, 0.063 ml, 0.25 mmol) was added dropwise to a solution of 1,1-dimethylethyl ((1S,2R)-1-{[6-(5-{5-cyano-6-[(1-methylethyl)oxy]-3-pyridinyl]-1,2,4-oxadiazol-3-yl)-5-methyl-3,4-dihydro-2(1H)-isoquinolinyl}carbonyl]-2-hydroxypropyl)carbamate (Preparation 100) (29 mg, 0.050 mmol) in dry DCM (0.5 ml) at 0° C. and the mixture stirred for 16 h at room temperature. The solvent was evaporated and the residue triturated using diethyl ether (1 ml). The solvent was decanted and the residue dried in vacuo to give 2-[(1-methylethyl)oxy]-5-[3-(5-methyl-2-L-threonyl-1,2,3,4-tetrahydro-6-isoquinolinyl)-1,2,4-oxadiazol-5-yl]-3-pyridinecarbonitrile hydrochloride as a colourless solid (18 mg)

LCMS (Method formate): Retention time 0.97 min, MH⁺=477

### Example 80

### 2-[7-(5-{3-cyano-4-[(1-methylethyl)oxy]phenyl}-1,2,4-oxadiazol-3-yl)-1,2,4,5-tetrahydro-3H-3-benzazepin-3-yl]-N-[(1S)-2-hydroxy-1-methylethyl]acetamide hydrochloride

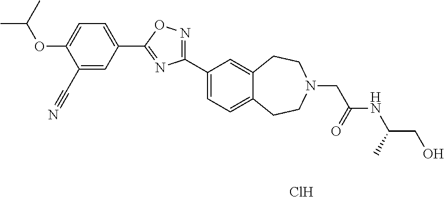

To a solution of 2-[7-(5-{3-cyano-4-[(1-methylethyl)oxy]phenyl}-1,2,4-oxadiazol-3-yl)-1,2,4,5-tetrahydro-3H-3-benzazepin-3-yl]-N-((1S)-2-{[(1,1-dimethylethyl)(dimethyl)silyl]oxy}-1-methylethyl)acetamide (Preparation 101) (121 mg, 0.2 mmol) in THF (2 ml) at room temperature under nitrogen was added tetrabutylammonium fluoride (TBAF, 1N in THF, 0.4 ml, 0.40 mmol) and the resulting mixture was stirred at this temperature for 1 h. The solvent was evaporated and the residue diluted with ethyl acetate. The mixture was washed with saturated sodium hydrogen carbonate (x2) dried (MgSO₄) and concentrated in vacuo. The residue was dissolved in 1,4-dioxane (2 ml) and treated with hydrogen chloride in 1,4-dioxane (4N, 1 ml). Removal of the solvent and trituration with diethyl ether gave a precipitate which was isolated by filtration to give 2-[7-(5-{3-cyano-4-[(1-methylethyl)oxy]phenyl}-1,2,4-oxadiazol-3-yl)-1,2,4,5-tetrahydro-3H-3-benzazepin-3-yl]-N-[(1S)-2-hydroxy-1-methylethyl]acetamide hydrochloride as a white solid (67 mg, 64%).

LCMS (Method HpH): Retention time 1.20 min, MH⁺=490

### Example 81

### 2-[(1-methylethyl)oxy]-5-{3-[3-(2-methyl-L-seryl)-2,3,4,5-tetrahydro-1H-3-benzazepin-7-yl]-1,2,4-oxadiazol-5-yl}benzonitrile

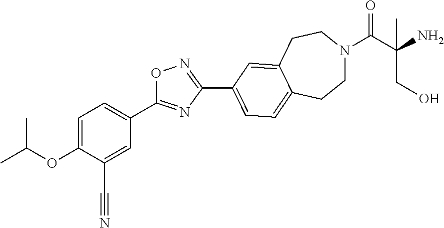

N-{[(1,1-Dimethylethyl)oxy]carbonyl}-2-methyl-L-serine (40 mg, 0.18 mmol), 2-[(1-methylethyl)oxy]-5-[3-(2,3,4,5-tetrahydro-1H-3-benzazepin-7-yl)-1,2,4-oxadiazol-5-yl]benzonitrile hydrochloride (Preparation 43) (50 mg, 0.12 mmol) and HATU (112 mg, 0.18 mmol) were combined with DMF (5 ml), and DIPEA (0.074 ml, 0.43 mmol). The mixture was stirred at room temperature for 2 h, partitioned between DCM (2×5 ml) and saturated sodium hydrogen carbonate solution (5 ml). The organic phases were combined, dried (hydrophobic frit) and evaporated in vacuo. The residue was dissolved in DCM (4 ml) and trifluoroacetic acid (1 m, 12.98 mmol) was added. The mixture was stirred at room temperature for 2 h and filtered through an aminopropyl SPE (5 g), washing the SPE with methanol in DCM (10%). The appropriate fractions were combined and evaporated in vacuo. The residue was dissolved in DMSO (2×1 ml) and purified by MDAP (Method formate). The appropriate fractions were filtered through an aminopropyl SPE (5 g) using methanol in DCM (10%). The appropriate fractions were combined and evaporated in vacuo to give 2-[(1-methylethyl)oxy]-5-{3-[3-(2-methyl-L-seryl)-2,3,4,5-tetrahydro-1H-3-benzazepin-7-yl]-1,2,4-oxadiazol-5-yl}benzonitrile as a white solid (12 mg).

LCMS (Method HpH): Retention time 1.16 min, MH⁺=476

### Example 82

### 2-[(1-methylethyl)oxy]-5-{3-[5-methyl-2-(2-methyl-L-seryl)-1,2,3,4-tetrahydro-6-isoquinolinyl]-1,2,4-oxadiazol-5-yl}benzonitrile

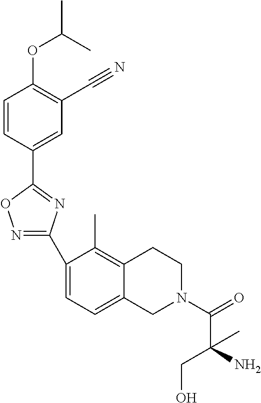

N-{[(1,1-Dimethylethyl)oxy]carbonyl}-2-methyl-L-serine (64 mg, 0.29 mmol), 2-[(1-methylethyl)oxy]-5-[3-(5-methyl-1,2,3,4-tetrahydro-6-isoquinolinyl)-1,2,4-oxadiazol-5-yl]benzonitrile hydrochloride (Preparation 25) (100 mg, 0.24 mmol) and HATU (139 mg, 0.37 mmol) were combined with DMF (5 ml), and DIPEA (0.149 ml, 0.85 mmol). The mixture was stirred at room temperature for 3 h, partitioned between DCM (2×5 ml) and saturated sodium hydrogen carbonate solution (5 ml). The organic phases were combined, dried (hydrophobic frit) and evaporated in vacuo. The residue was dissolved in DCM (4 ml), and trifluoroacetic acid (1 ml, 13 mmol) was added. The mixture was stirred at room temperature for 2 h, and filtered through an aminopropyl SPE (5 g), washing the SPE with methanol in DCM (10%). The appropriate fractions were combined and evaporated in vacuo. The residue was dissolved in DMSO (2×1 ml) and purified by MDAP (Method HpH). The appropriate fractions were concentrated under a stream of nitrogen. The residue was dissolved in DMSO (1 ml) and purified by MDAP (Method formate). The appropriate fractions were filtered through an aminopropyl SPE (5 g) using methanol in DCM (10%). The appropriate fractions were combined and evaporated in vacuo to give 2-[(1-methylethyl)oxy]-5-{3-[5-methyl-2-(2-methyl-L-seryl)-1,2,3,4-tetrahydro-6-isoquinolinyl]-1,2,4-oxadiazol-5-yl}benzonitrile as a white solid (50 mg).

LCMS (Method HpH): Retention time 1.16 min, MH⁺=476

### Example 83

### 5-(3-{3-[4-(methylamino)butanoyl]-2,3,4,5-tetrahydro-1H-3-benzazepin-7-yl}-1,2,4-oxadiazol-5-yl)-2-[(1-methylethyl)oxy]benzonitrile

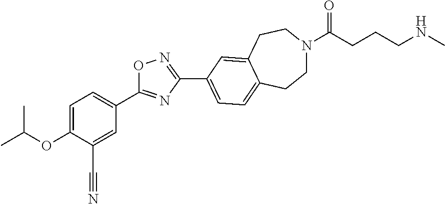

4-[{[(1,1-Dimethylethyl)oxy]carbonyl}(methyl)amino)butanoic acid (64 mg, 0.29 mmol), 2-[(1-methylethyl)oxy]-5-[3-(2,3,4,5-tetrahydro-1H-3-benzazepin-7-yl)-1,2,4-oxadiazol-5-yl]benzonitrile hydrochloride (Preparation 43) (100 mg, 0.24 mmol) and HATU (139 mg, 0.37 mmol) were combined with DMF (5 ml), and DIPEA (0.149 ml, 0.85 mmol). The mixture was stirred at room temperature for 3 h, partitioned between DCM (2×5 ml) and saturated sodium hydrogen carbonate solution (5 ml). The organic phases were combined, dried (hydrophobic frit) and evaporated in vacuo. The residue was dissolved in DCM (4 ml), and trifluoroacetic acid (1 ml, 13 mmol) was added. The mixture was stirred at room temperature for 2 h, and filtered through an aminopropyl SPE (5 g), washing the SPE with methanol in DCM (10%). The appropriate fractions were combined and evaporated in vacuo. The residue was dissolved in DMSO (2×1 ml) and purified by MDAP (Method HpH). The solvent was evaporated under a stream of nitrogen to give 5-(3-{3-[4-(methylamino)butanoyl]-2,3,4,5-tetrahydro-1H-3-benzazepin-7-yl}-1,2,4-oxadiazol-5-yl)-2-[(1-methylethyl)oxy]benzonitrile as a yellow gum (82 mg).

LCMS (Method HpH): Retention time 1.16 min, MH⁺=474

### Example 84

### 2-[(1-methylethyl)oxy]-5-{3-[3-(2-methyl-D-seryl)-2,3,4,5-tetrahydro-1H-3-benzazepin-7-yl]-1,2,4-oxadiazol-5-yl}benzonitrile

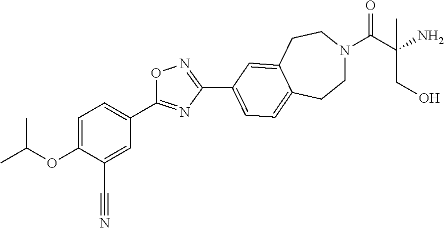

N-{[(1,1-Dimethylethyl)oxy]carbonyl}-2-methyl-D-serine (64 mg, 0.29 mmol), 2-[(1-methylethyl)oxy]-5-[3-(2,3,4,5-tetrahydro-1H-3-benzazepin-7-yl)-1,2,4-oxadiazol-5-yl]benzonitrile hydrochloride (Preparation 43) (100 mg, 0.24 mmol) and HATU (139 mg, 0.37 mmol) were combined DMF (5 ml), and DIPEA (0.149 ml, 0.85 mmol). The mixture was stirred at room temperature for 3 h, partitioned between DCM (2×5 ml) and saturated sodium hydrogen carbonate solution (5 ml). The organic phases were combined, dried (hydrophobic frit) and evaporated in vacuo. The residue was dissolved in DCM (4 ml), and trifluoroacetic acid (1 ml, 13 mmol) was added. The mixture was stirred at room temperature for 2 h, and filtered through an aminopropyl SPE (5 g), washing the SPE with methanol in DCM (10%). The appropriate fractions were combined and evaporated in vacuo. The residue was dissolved in DMSO (2×1 ml) and purified by MDAP (Method HpH). The solvent was evaporated under a stream of nitrogen to give 2-[(1-methylethyl)oxy]-5-{3-[3-(2-methyl-D-seryl)-2,3,4,5-tetrahydro-1H-3-benzazepin-7-yl]-1,2,4-oxadiazol-5-yl}benzonitrile as a colourless gum (67 mg).

LCMS (Method HpH): Retention time 1.16 min, MH⁺=476

### Example 85

### 2-[(1-methylethyl)oxy]-5-{3-[5-methyl-2-(2-methyl-D-seryl)-1,2,3,4-tetrahydro-6-isoquinolinyl]-1,2,4-oxadiazol-5-yl}benzonitrile

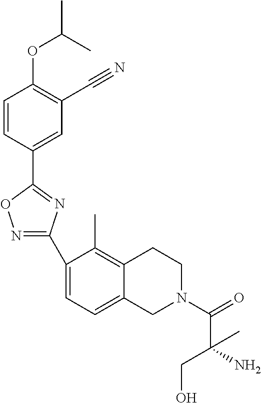

N-{[(1,1-Dimethylethyl)oxy]carbonyl}-2-methyl-D-serine (64 mg, 0.29 mmol), 2-[(1-methylethyl)oxy]-5-[3-(5-methyl-1,2,3,4-tetrahydro-6-isoquinolinyl)-1,2,4-oxadiazol-5-yl]benzonitrile hydrochloride (Preparation 25) (100 mg, 0.24 mmol) and HATU (139 mg, 0.37 mmol) were combined with DMF (5 ml), and DIPEA (0.149 ml, 0.85 mmol). The mixture was stirred at room temperature for 3 h, partitioned between DCM (2×5 ml) and saturated sodium hydrogen carbonate solution (5 ml). The organic phases were combined, dried (hydrophobic frit) and evaporated in vacuo. The residue was dissolved in DCM (4 ml), and trifluoroacetic acid (1 ml, 12.98 mmol) was added. The mixture was stirred at room temperature for 2 h, and filtered through an aminopropyl SPE (5 g), washing the SPE with methanol in DCM (10%). The appropriate fractions were combined and evaporated in vacuo. The residue was dissolved in DMSO (2×1 ml) and purified by MDAP (Method TFA). The appropriate fractions were filtered through an aminopropyl SPE (5 g), washing the SPE with methanol in DCM (10%). The appropriate fractions were combined and evaporated in vacuo to give 2-[(1-methylethyl)oxy]-5-{3-[5-methyl-2-(2-methyl-D-seryl)-1,2,3,4-tetrahydro-6-isoquinolinyl]-1,2,4-oxadiazol-5-yl}benzonitrile (67 mg).

LCMS (Method HpH): Retention time 1.16 min, MH⁺=476

### Example 86

### 5-(3-{3-[N-(2-hydroxyethyl)glycyl]-2,3,4,5-tetrahydro-1H-3-benzazepin-7-yl}-1,2,4-oxadiazol-5-yl)-2-[(1-methylethyl)oxy]benzonitrile hydrochloride

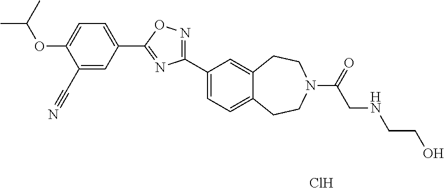

To a solution of 5-{3-[3-(bromoacetyl)-2,3,4,5-tetrahydro-1H-3-benzazepin-7-yl}-1,2,4-oxadiazol-5-yl}-2-[(1-methylethyl)oxy]benzonitrile (Preparation 102) (99 mg, 0.2 mmol) in acetonitrile (3 ml) at room temperature under nitrogen was added potassium carbonate (42 mg, 0.30 mmol) then ethanolamine (0.013 ml, 0.22 mmol) and the resulting mixture was stirred at 60° C. for 30 min. The reaction was cooled to room temperature and concentrated in vacuo. The residue was dissolved in ethyl acetate and saturated aqueous sodium hydrogen carbonate solution, and the layers were separated. The aqueous phase was extracted with ethyl acetate (x2), and the combined organic layers were dried (MgSO₄) and concentrated in vacuo. The residue was purified by MDAP. The resulting pale grey foam was suspended in 1,4-dioxane (2 ml) to which was added hydrogen chloride in 1,4-dioxane (4N, 0.5 ml). Removal of the solvent, coevaporation and then trituration with diethyl ether gave a white solid which was isolated by filtration and dried under vacuum to give 5-(3-{3-N-(2-hydroxyethyl)glycyl]-2,3,4,5-tetrahydro-1H-3-benzazepin-7-yl}-1,2,4-oxadiazol-5-yl)-2-[(1-methylethyl)oxy]benzonitrile hydrochloride (15 mg, 15%).

LCMS (Method formate): Retention time 0.90 min, MH⁺=476

### Example 87

### 5-[3-(2-glycyl-1,2,3,4-tetrahydro-5-isoquinolinyl)-1,2,4-oxadiazol-5-yl]-2-[(1-methylethyl)oxy]benzonitrile hydrochloride

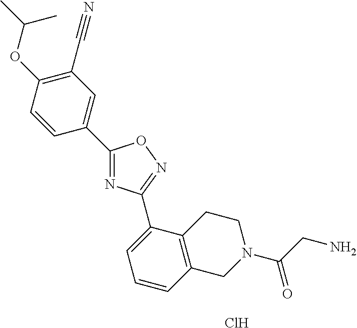

A mixture of 1,1-dimethylethyl {2-[5-(5-{3-cyano-4-[(1-methylethyl)oxy]phenyl}-1,2,4-oxadiazol-3-yl)-3,4-dihydro-2(1H)-isoquinolinyl]-2-oxoethyl}carbamate (Preparation 103) (150 mg, 0.29 mmol), 1,4-dioxane (0.75 ml) and hydrogen chloride in 1,4-dioxane (4M, 0.5 ml) was stirred at room temperature for 2 h. Ether (10 ml) was added. After stirring for 15 min the precipitate was isolated by filtration, washed with diethyl ether and dried to give 5-[3-(2-glycyl-1,2,3,4-tetrahydro-5-isoquinolinyl)-1,2,4-oxadiazol-5-yl]-2-[(1-methylethyl)oxy]benzonitrile hydrochloride as a colourless solid (102 mg).

LCMS (Method formate): Retention time 0.89 min, MH⁺=418

Example 88 543-(2-β-alanyl-1,2,3,4-tetrahydro-5-isoquinolinyl)-1,2,4-oxadiazol-5-yl]-2-[(1-methylethyl)oxy]benzonitrile hydrochloride

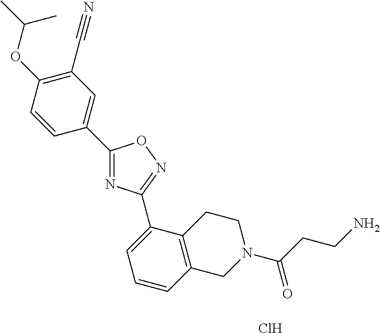

A mixture of 1,1-dimethylethyl {3-[5-(5-{3-cyano-4-[(1-methylethyl)oxy]phenyl}-1,2,4-oxadiazol-3-yl)-3,4-dihydro-2(1H)-isoquinolinyl]-3-oxopropyl}carbamate (Preparation 104) (140 mg, 0.26 mmol), 1,4-dioxane (0.75 ml) and hydrogen chloride in 1,4-dioxane (4M, 0.5 ml) was stirred at room temperature for 2 h. Ether (10 ml) was added. After stirring for 15 min the precipitate was isolated by filtration, washed with diethyl ether and dried to give 5-[3-(2-β-alanyl-1,2,3,4-tetrahydro-5-isoquinolinyl)-1,2,4-oxadiazol-5-yl]-2-[(1-methylethyl)oxy]benzonitrile hydrochloride as a colourless solid (106 mg).

LCMS (Method formate): Retention time 0.89 min, MH⁺=432

### Example 89

### 2-[(1-methylethyl)oxy]-5-[3-(2-L-seryl-1,2,3,4-tetrahydro-5-isoquinolinyl)-1,2,4-oxadiazol-5-yl]benzonitrile hydrochloride

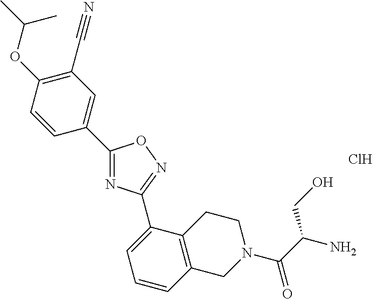

A mixture of 1,1-dimethylethyl [(1S)-2-[5-(5-{3-cyano-4-[(1-methylethyl)oxy]phenyl}-1,2,4-oxadiazol-3-yl)-3,4-dihydro-2(1H)-isoquinolinyl]-1-(hydroxymethyl)-2-oxoethyl]carbamate (Preparation 105) (130 mg, 0.24 mmol), 1,4-dioxane (0.75 ml) and hydrogen chloride in 1,4-dioxane (4M, 0.5 ml) was stirred at room temperature for 2 h. Ether (10 ml) was added. After stirring for 15 min the precipitate was isolated by filtration, washed with diethyl ether and dried to give 2-[(1-methylethyl)oxy]-5-[3-(2-L-seryl-1,2,3,4-tetrahydro-5-isoquinolinyl)-1,2,4-oxadiazol-5-yl]benzonitrile hydrochloride as a colourless solid (86 mg).

LCMS (Method formate): Retention time 0.89 min, MH⁺=448

### Example 90

### 2-[(1-methylethyl)oxy]-5-[3-(2-D-seryl-1,2,3,4-tetrahydro-5-isoquinolinyl)-1,2,4-oxadiazol-5-yl]benzonitrile hydrochloride

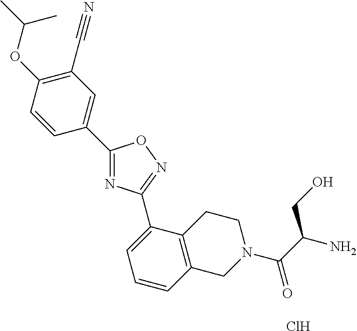

A mixture of 1,1-dimethylethyl [(1R)-2-[5-(5-{3-cyano-4-[(1-methylethyl)oxy]phenyl}-1,2,4-oxadiazol-3-yl)-3,4-dihydro-2(1H)-isoquinolinyl]-1-(hydroxymethyl)-2-oxoethyl]carbamate (Preparation 106) (125 mg, 0.23 mmol), 1,4-dioxane (0.75 ml) and hydrogen chloride in 1,4-dioxane (4M, 0.5 ml) was stirred at room temperature for 2 h. Ether (10 ml) was added. After stirring for 15 min the precipitate was isolated by filtration, washed with diethyl ether and dried to give 2-[(1-methylethyl)oxy]-5-[3-(2-D-seryl-1,2,3,4-tetrahydro-5-isoquinolinyl)-1,2,4-oxadiazol-5-yl]benzonitrile hydrochloride as a colourless solid (91 mg).

LCMS (Method formate): Retention time 0.89 min, MH⁺=448

### Example 91

### 2-[(1-methylethyl)oxy]-5-{3-[3-(4-morpholinylacetyl)-2,3,4,5-tetrahydro-1H-3-benzazepin-7-yl]-1,2,4-oxadiazol-5-yl}benzonitrile hydrochloride

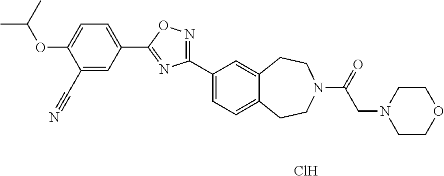

To a solution of 5-{3-[3-(bromoacetyl)-2,3,4,5-tetrahydro-1H-3-benzazepin-7-yl}-1,2,4-oxadiazol-5-yl}-2-[(1-methylethyl)oxy]benzonitrile (Preparation 102) (99 mg, 0.2 mmol) in acetonitrile (3 ml) at room temperature under nitrogen was added potassium carbonate (42 mg, 0.30 mmol) then morpholine (0.035 ml, 0.40 mmol) and the resulting mixture was stirred at 60° C. for 20 min. The reaction was cooled to room temperature and concentrated in vacuo. The residue was dissolved in ethyl acetate and saturated aqueous sodium hydrogen carbonate solution and the layers were separated. The aqueous phase was extracted with ethyl acetate (x2), the combined organic layers were dried (MgSO₄) and concentrated in vacuo. The residue was dissolved in 1,4-dioxane (2 ml), treated with hydrogen chloride in 1,4-dioxane (4N) and the mixture was concentrated in vacuo. The residue was coevaporated with diethyl ether then triturated with diethyl ether. The white solid formed was isolated by filtration and dried under vacuum. The solid was purified by MDAP (Method HpH). The residue was dissolved in 1,4-dioxane (2 ml), treated with hydrogen chloride in 1,4-dioxane (4N) and the mixture was concentrated in vacuo to give 24(1-methylethyl)oxy]-5-{3-[3-(4-morpholinylacetyl)-2,3,4,5-tetrahydro-1H-3-benzazepin-7-yl]-1,2,4-oxadiazol-5-yl}benzonitrile hydrochloride as a white solid (35 mg, 33%).

LCMS (Method HpH): Retention time 1.23 min, MH⁺=502

### Example 92

### 2-[(1-methylethyl)oxy]-5-(3-{3-[2-(4-morpholinyl)-2-oxoethyl]-2,3,4,5-tetrahydro-1H-3-benzazepin-7-yl}-1,2,4-oxadiazol-5-yl)benzonitrile hydrochloride

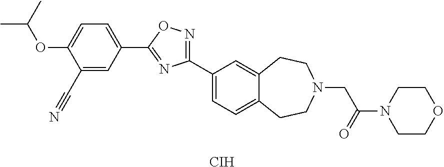

To a solution of [7-(5-{3-cyano-4-[(1-methylethyl)oxy]phenyl}-1,2,4-oxadiazol-3-yl)-1,2,4,5-tetrahydro-3H-3-benzazepin-3-yl]acetic acid (Preparation 89) (86 mg, 0.2 mmol) in DMF (3 ml) at room temperature were added EDC (46 mg, 0.24 mmol), N-ethylmorpholine (0.076 ml, 0.60 mmol) and HOBT (37 mg, 0.24 mmol) then after 2 min morpholine (0.026 ml, 0.30 mmol) and the resulting mixture was stirred 16 h at room temperature. Further portions of EDC (46 mg, 0.24 mmol), N-ethylmorpholine (0.076 ml, 0.60 mmol) and HOBT (37 mg, 0.24 mmol) and morpholine (0.026 ml, 0.30 mmol) were added and the resulting mixture stirred for 16 h then concentrated in vacuo. The residue was diluted in ethyl acetate and the organic phase washed with saturated sodium hydrogen carbonate (x2), dried (MgSO₄) and concentrated in vacuo. The residue was dissolved in 1,4-dioxane (2 ml) and treated with treated with hydrogen chloride in 1,4-dioxane (4N) and the mixture was concentrated in vacuo. The residue was coevaporated with diethyl ether then triturated with diethyl ether. The white solid was isolated by filtration and dried under vacuum to give 2-[(1-methylethyl)oxy]-5-(3-{3-[2-(4-morpholinyl)-2-oxoethyl]-2,3,4,5-tetrahydro-1H-3-benzazepin-7-yl}-1,2,4-oxadiazol-5-yl)benzonitrile hydrochloride (44 mg, 41%).

LCMS (Method HpH): Retention time 1.27 min, MH⁺=502

### Example 93

### 5-(3-{3-[2-(3-hydroxy-1-azetidinyl)-2-oxoethyl]-2,3,4,5-tetrahydro-1H-3-benzazepin-7-yl}-1,2,4-oxadiazol-5-yl)-2-[(1-methylethyl)oxy]benzonitrile hydrochloride

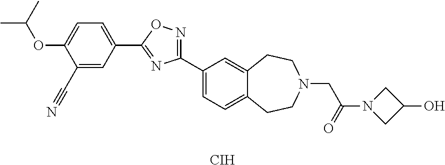

A solution of crude 5-(3-{3-[2-(3-{[(1,1-dimethylethyl)(dimethyl)silyl]oxy}-1-azetidinyl)-2-oxoethyl]-2,3,4,5-tetrahydro-1H-3-benzazepin-7-yl}-1,2,4-oxadiazol-5-yl)-2-[(1-methylethyl)oxy]benzonitrile (Preparation 107) (120 mg, 0.2 mmol) in THF (2 ml) at room temperature was treated with tetrabutyl ammonium fluoride (1N in THF, 0.105 ml, 0.40 mmol) and the resulting mixture was stirred for 1 h then concentrated in vacuo. The residue was dissolved in 1,4-dioxane (2 ml) and treated with treated with hydrogen chloride in 1,4-dioxane (4N, 1 ml) then concentrated in vacuo. The residue was triturated with diethyl ether and the solid was isolated by filtration. The solid was further was purified by MDAP (Method HpH). The residue was The residue was dissolved in 1,4-dioxane and treated with treated with hydrogen chloride in 1,4-dioxane (4N) then concentrated in vacuo to give 5-(3-{3-[2-(3-hydroxy-1-azetidinyl)-2-oxoethyl]-2,3,4,5-tetrahydro-1H-3-benzazepin-7-yl}-1,2,4-oxadiazol-5-yl)-2-[(1-methylethyl)oxy]benzonitrile hydrochloride as a white solid (22 mg).

LCMS (Method HpH): Retention time 1.12 min, MH⁺=488

### Example 94

### 2-(3-fluoro-1-azetidinyl)-5-[3-(3-glycyl-2,3,4,5-tetrahydro-1H-3-benzazepin-7-yl)-1,2,4-oxadiazol-5-yl]benzonitrile

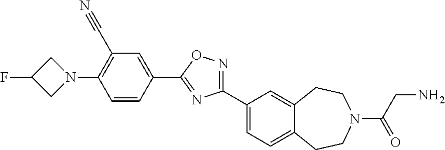

N-{[(1,1-dimethylethyl)oxy]carbonyl}glycine (54 mg, 0.31 mmol), 2-(3-fluoro-1-azetidinyl)-5-[3-(2,3,4,5-tetrahydro-1H-3-benzazepin-7-yl)-1,2,4-oxadiazol-5-yl]benzonitrile (Preparation 108) (100 mg, 0.26 mmol), HATU (146 mg, 0.39 mmol) were suspended in DMF (5 ml), and DIPEA (0.157 ml, 0.90 mmol) added to the mixture. The reaction was stirred at room temperature for 2 h, partitioned between DCM (2×5 ml) and saturated sodium hydrogen carbonate solution (5 ml). The organic phases were combined, (hydrophobic frit) and reduced to dryness under a stream of nitrogen. The residue was dissolved in DCM (4 ml) and trifluoroacetic acid (1 ml, 12.98 mmol) was added. The reaction mixture was stirred at room temperature for 1 h. The reaction mixture was concentrated, dissolved in DCM and applied to an aminopropyl SPE (10 g) which was eluted with methanol in DCM (10%). The appropriate fractions were combined and reduced to dryness under a stream of nitrogen. The residue was dissolved in DMSO (3×1 ml) and purified by MDAP (Method HpH). The solvent was evaporated in vacuo to give 2-(3-fluoro-1-azetidinyl)-5-[3-(3-glycyl-2,3,4,5-tetrahydro-1H-3-benzazepin-7-yl)-1,2,4-oxadiazol-5-yl]benzonitrile as a grey solid (82 mg).

LCMS (Method HpH): Retention time 1.04 min, MH⁺=447

### Example 95

### 5-(3-{3-[2-hydroxy-1-(hydroxymethyl)ethyl]-2,3,4,5-tetrahydro-1H-3-benzazepin-7-yl}-1,2,4-oxadiazol-5-yl)-2-[(1-methylethyl)oxy]-3-pyridinecarbonitrile

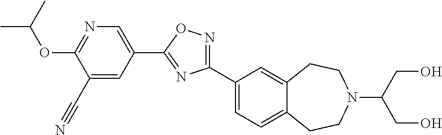

To a stirred solution the impure 5-{3-[3-(2,2-dimethyl-1,3-dioxan-5-yl)-2,3,4,5-tetrahydro-1H-3-benzazepin-7-yl]-1,2,4-oxadiazol-5-yl}-2-[(1-methylethyl)oxy]-3-pyridinecarbonitrile (Preparation 110) (230 mg, 0.47 mmol max.) in THF (6 ml) was added hydrochloric acid (2M, 6.0 ml, 18 mmol). The mixture was stirred at room temperature for 16 h. The volatiles were evaporated under a stream of nitrogen and the residue was partitioned between saturated aqueous sodium bicarbonate solution (5 ml) and ethyl acetate (5 ml). The aqueous phase was extracted with ethyl acetate (3×4 ml). The combined organic phases were dried (hydrophobic frit) and evaporated to dryness under a stream of nitrogen. The residue was purified by MDAP (Method formate). The required fraction was evaporated under a stream of nitrogen to give 5-(3-{3-[2-hydroxy-1-(hydroxymethyl)ethyl]-2,3,4,5-tetrahydro-1H-3-benzazepin-7-yl}-1,2,4-oxadiazol-5-yl)-2-[(1-methylethyl)oxy]-3-pyridinecarbonitrile as a white crunchy foam, (77 mg, 37%)

LCMS (Method formate): Retention time 0.93 min, MH⁺=450

### Example 96

### 5-(3-{3-[(2S)-2,3-dihydroxypropyl]-2,3,4,5-tetrahydro-1H-3-benzazepin-7-yl}-1,2,4-oxadiazol-5-yl)-2-[(1-methylethyl)oxy]-3-pyridinecarbonitrile

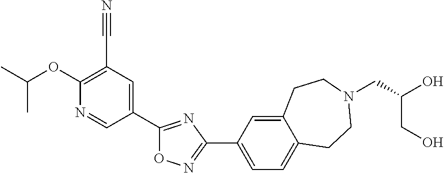

To a stirred suspension of L-(−)-glyceraldehyde (270 mg, 3.0 mmol) and 2-[(1-methylethyl)oxy]-5-[3-(2,3,4,5-tetrahydro-1H-3-benzazepin-7-yl)-1,2,4-oxadiazol-5-yl]-3-pyridinecarbonitrile trifluoroacetate (Preparation 96) (245 mg, 0.5 mmol), in DCM (10 ml) under nitrogen, was added acetic acid (0.043 ml, 0.75 mmol) followed by sodium triacetoxyborohydride (689 mg, 3.3 mmol). The mixture was stirred at room temperature for 115 h. Saturated aqueous sodium hydrogen carbonate solution (5 ml) was added and the reaction stirred vigorously for 15 min. The mixture was evaporated to dryness under a stream of nitrogen and the residue partitioned between saturated aqueous sodium hydrogen carbonate solution (5 ml) and ethyl acetate (5 ml). The aqueous phase was extracted with ethyl acetate (3×4 ml). The combined organic phases were dried (hydrophobic frit) and evaporated to dryness under a stream of nitrogen. The residue was purified by MDAP (Method formate).

The required fraction was evaporated under a stream of nitrogen and the residue further purified by MDAP (Method formate, extended run). The required fraction was evaporated under a stream of nitrogen to give 5-(3-{3-[(2S)-2,3-dihydroxypropyl]-2,3,4,5-tetrahydro-1H-3-benzazepin-7-yl}-1,2,4-oxadiazol-5-yl)-2-[(1-methylethyl)oxy]-3-pyridinecarbonitrile as a white solid (63 mg, 28%).

LCMS (Method formate): Retention time 0.93 min, MH⁺=450

### Example 97

### 5-(3-{3-[(2R)-2,3-dihydroxypropyl]-2,3,4,5-tetrahydro-1H-3-benzazepin-7-yl}-1,2,4-oxadiazol-5-yl)-2-[(1-methylethyl)oxy]-3-pyridinecarbonitrile

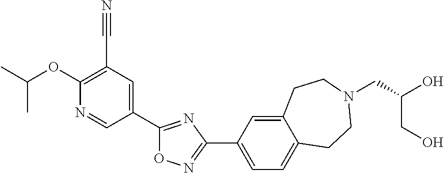

To a stirred suspension of D-(+)-glyceraldehyde (270 mg, 3.0 mmol) and 2-[(1-methylethyl)oxy]-5-[3-(2,3,4,5-tetrahydro-1H-3-benzazepin-7-yl)-1,2,4-oxadiazol-5-yl]-3-pyridinecarbonitrile trifluoroacetate (Preparation 96) (245 mg, 0.5 mmol), in DCM (10 ml) under nitrogen, was added acetic acid (0.043 ml, 0.75 mmol) followed by sodium triacetoxyborohydride (689 mg, 3.3 mmol). The mixture was stirred at room temperature for 115 h. Saturated aqueous sodium hydrogen carbonate solution (5 ml) was added and the reaction stirred vigorously for 15 min. The mixture was evaporated to dryness under a stream of nitrogen and the residue partitioned between saturated aqueous sodium hydrogen carbonate solution (5 ml) and ethyl acetate (5 ml). The aqueous phase was extracted with ethyl acetate (3×4 ml). The combined organic phases were dried (hydrophobic frit) and evaporated to dryness under a stream of nitrogen. The residue was purified by MDAP (Method formate). The required fraction was evaporated under a stream of nitrogen and the residue further purified by MDAP (Method formate, extended run). The required fraction was evaporated under a stream of nitrogen to give 5-(3-{3-[(25)-2,3-dihydroxypropyl]-2,3,4,5-tetrahydro-1H-3-benzazepin-7-yl}-1,2,4-oxadiazol-5-yl)-2-[(1-methylethyl)oxy]-3-pyridinecarbonitrile as a clear gum (86 mg, 38%).

LCMS (Method formate): Retention time 0.92 min, MH⁺=450

### Example 98

### 5-[3-(3-{N-(1R)-2-hydroxy-1-methylethyl]glycyl}-2,3,4,5-tetrahydro-1H-3-benzazepin-7-yl)-1,2,4-oxadiazol-5-yl]-2-[(1-methylethyl)oxy]benzonitrile hydrochloride

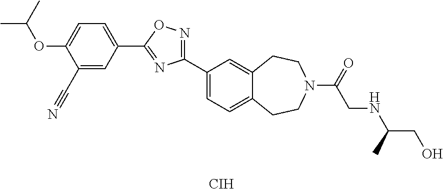

To a solution of 5-{3-[3-(bromoacetyl)-2,3,4,5-tetrahydro-1H-3-benzazepin-7-yl}-1,2,4-oxadiazol-5-yl}-2-[(1-methylethyl)oxy]benzonitrile (Preparation 102) (99 mg, 0.2 mmol) in acetonitrile (3 ml) at room temperature under nitrogen was added potassium carbonate (41 mg, 0.30 mmol) then (2R)-2-amino-1-propanol (0.078 ml, 1.0 mmol) and the resulting mixture was stirred at 60° C. for 20 min. The reaction was cooled to room temperature, most of the solvent removed and the residue partitioned between ethyl acetate and saturated sodium hydrogen carbonate. The organic phase was washed with saturated sodium hydrogen carbonate, dried (MgSO₄) and concentrated in vacuo. The residue was purified by MDAP (Method HpH). The purified material was dissolved in 1,4-dioxane (2 ml) and treated with hydrogen chloride in 1,4-dioxane (1 ml). The mixture was concentrated in vacuo, the residue triturated with diethyl ether and the solid isolated by filtration to give 5-[3-(3-{N-(1R)-2-hydroxy-1-methylethyl]glycyl}-2,3,4,5-tetrahydro-1H-3-benzazepin-7-yl)-1,2,4-oxadiazol-5-yl]-2-[(1-methylethyl)oxy]benzonitrile hydrochloride (27 mg, 26%) as a white solid.

LCMS (Method HpH): Retention time 1.14 min, MH⁺=490

### Example 99

### 5-[3-(3-{N-[(1S)-2-hydroxy-1-methylethyl]glycyl}-2,3,4,5-tetrahydro-1H-3-benzazepin-7-yl)-1,2,4-oxadiazol-5-yl]-2-[(1-methylethyl)oxy]benzonitrile hydrochloride

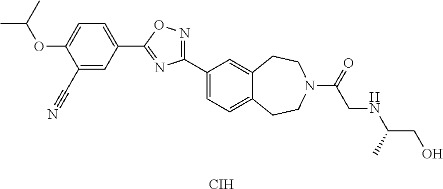

To a solution of 5-{3-[3-(bromoacetyl)-2,3,4,5-tetrahydro-1H-3-benzazepin-7-yl}-1,2,4-oxadiazol-5-yl}-2-[(1-methylethyl)oxy]benzonitrile (Preparation 102) (99 mg, 0.2 mmol) in acetonitrile (3 ml) at room temperature under nitrogen was added potassium carbonate (42 mg, 0.30 mmol) then (2S)-2-amino-1-propanol (15 mg) and the resulting mixture was stirred at 60° C. for 20 min. The reaction was cooled to room temperature, most of the solvent removed and the residue partitioned between ethyl acetate and saturated sodium hydrogen carbonate. The organic phase was washed with saturated sodium hydrogen carbonate, dried (MgSO₄) and concentrated in vacuo. The residue was purified by MDAP (Method HpH). The purified material was dissolved in 1,4-dioxane (2 ml) and treated with hydrogen chloride in 1,4-dioxane (1 ml). The mixture was concentrated in vacuo, the residue triturated with diethyl ether and the solid isolated by filtration to give 5-[3-(3-{N-[(1S)-2-hydroxy-1-methylethyl]glycyl}-2,3,4,5-tetrahydro-1H-3-benzazepin-7-yl)-1,2,4-oxadiazol-5-yl]-2-[(1-methylethyl)oxy]benzonitrile hydrochloride (32 mg, 30%) as a white solid.

LCMS (Method HpH): Retention time 1.14 min, MH⁺=490

### Example 100

### methyl {2-[7-(5-{3-cyano-4-[(1-methylethyl)oxy]phenyl}-1,2,4-oxadiazol-3-yl)-1,2,4,5-tetrahydro-3H-3-benzazepin-3-yl]ethyl}carbamate

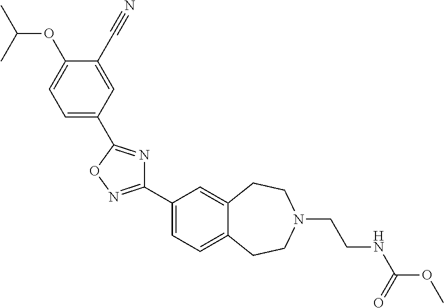

5-{3-[3-(2-Aminoethyl)-2,3,4,5-tetrahydro-1H-3-benzazepin-7-yl]-1,2,4-oxadiazol-5-yl}-2-[(1-methylethyl)oxy]benzonitrile hydrochloride (Preparation 111) (100 mg, 0.20 mmol) was dissolved in a mixture of DCM (2 ml) and pyridine (1 ml). The solution was stirred at room temperature and methyl chloridocarbonate (0.024 ml, 0.31 mmol) was added. The solution was stirred for 30 min. The solvents were removed under vacuum and water (10 ml) added to the residue. The mixture was extracted using ethyl acetate (3×10 ml). The ethyl acetate dried (hydrophobic frit) and reduced to dryness under vacuum. The residue was dissolved in DMSO/methanol (1:1, 2 ml) and purified by MDAP (Method formate), which gave methyl {2-[7-(5-{3-cyano-4-[(1-methylethyl)oxy]phenyl}-1,2,4-oxadiazol-3-yl)-1,2,4,5-tetrahydro-3H-3-benzazepin-3-yl]ethyl}carbamate as an off-white solid (23 mg)

LCMS (Method formate): Retention time 0.93 min, MH⁺=476

### Example 101

### N-{2-[7-(5-{3-cyano-4-[(1-methylethyl)oxy]phenyl}-1,2,4-oxadiazol-3-yl)-1,2,4,5-tetrahydro-3H-3-benzazepin-3-yl]ethyl}acetamide

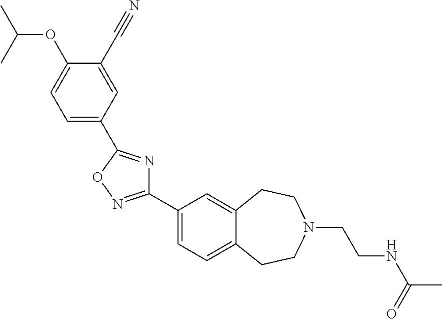

5-{3-[3-(2-aminoethyl)-2,3,4,5-tetrahydro-1H-3-benzazepin-7-yl]-1,2,4-oxadiazol-5-yl}-2-[(1-methylethyl)oxy]benzonitrile hydrochloride (Preparation 111) (100 mg, 0.20 mmol) was dissolved in a mixture of pyridine (1 ml) and DCM (2 ml). The solution was stirred at room temperature and acetyl chloride (0.019 ml, 0.31 mmol) was added. The solution was stirred for 30 min. The solvents were evaporated under vacuum and water (10 ml) added to the residue. The mixture was extracted using ethyl acetate (3×10 ml). The ethyl acetate dried (hydrophobic frit) and reduced to dryness under vacuum. The residue was dissolved in DMSO/methanol (1:1, 2 ml) and purified by MDAP (Method formate) which gave N-{2-[7-(5-{3-cyano-4-[(1-methylethyl)oxy]phenyl}-1,2,4-oxadiazol-3-yl)-1,2,4,5-tetrahydro-3H-3-benzazepin-3-yl]ethyl}acetamide as an off white solid (25 mg).

LCMS (Method formate): Retention time 0.83 min, MH⁺=460

### Example 102

### methyl {3-[7-(5-{3-cyano-4-[(1-methylethyl)oxy]phenyl}-1,2,4-oxadiazol-3-yl)-1,2,4,5-tetrahydro-3H-3-benzazepin-3-yl]propyl}carbamate

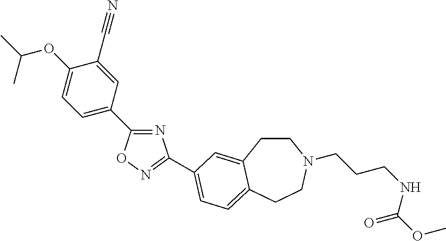

5-{3-[3-(3-aminopropyl)-2,3,4,5-tetrahydro-1H-3-benzazepin-7-yl]-1,2,4-oxadiazol-5-yl}-2-[(1-methylethyl)oxy]benzonitrile hydrochloride (Preparation 113) (200 mg, 0.43 mmol) was dissolved in a mixture of DCM (4 ml) and pyridine (2 ml) and stirred at room temperature. Methyl chloridocarbonate (40 mg, 0.43 mmol) was added and the solution was stirred for 20 min. The solvent were evaporated under vacuum. Water (10 ml) was added to the residue and the mixture was extracted using ethyl acetate (3×8 ml). The ethyl acetate was dried (hydrophobic frit) and evaporated under vacuum. The residue was dissolved in a mixture of DMSO/methanol (1:1, 1 ml) and purified by MDAP (Method formate) which gave methyl {3-[7-(5-{3-cyano-4-[(1-methylethyl)oxy]phenyl}-1,2,4-oxadiazol-3-yl)-1,2,4,5-tetrahydro-3H-3-benzazepin-3-yl]propyl}carbamate as a brown solid (49 mg).

LCMS (Method formate): Retention time 0.92 min, MH⁺=490

### Example 103

Formic acid-N-{3-[7-(5-{3-cyano-4-[(1-methylethyl)oxy]phenyl}-1,2,4-oxadiazol-3-yl)-1,2,4,5-tetrahydro-3H-3-benzazepin-3-yl]propyl}propanamide (1:1)

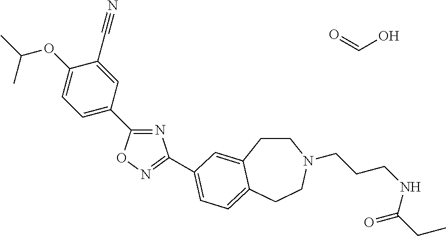

5-{3-[3-(3-Aminopropyl)-2,3,4,5-tetrahydro-1H-3-benzazepin-7-yl]-1,2,4-oxadiazol-5-yl}-2-[(1-methylethyl)oxy]benzonitrile (Preparation 113) (200 mg, 0.43 mmol) was dissolved in a mixture of DCM (4 ml) and pyridine (2 ml) and stirred at room temperature. Methyl chloridocarbonate (40 mg, 0.43 mmol) was added and the solution stirred for 20 min. The volatiles were evaporated under vacuum. Water (10 ml) added to the residue and the mixture extracted using ethyl acetate (3×8 ml). The combined organic phases were dried (hydrophobic frit) and the solvent evaporated under vacuum. The residue was dissolved in DMSO/methanol (1:1, 1 ml) and purified by MDAP (Method formate) to give formic acid-N-{3-[7-(5-{3-cyano-4-[(1-methylethyl)oxy]phenyl}-1,2,4-oxadiazol-3-yl)-1,2,4,5-tetrahydro-3H-3-benzazepin-3-yl]propyl}propanamide (1:1) as a dark brown solid (116 mg).

LCMS (Method formate): Retention time 0.97 min, MH⁺=488

### Example 104

### 5-{3-[3-(2,3-dihydroxypropyl)-2,3,4,5-tetrahydro-1H-3-benzazepin-7-yl]-1,2,4-oxadiazol-5-yl}-2-(3-fluoro-1-azetidinyl)benzonitrile

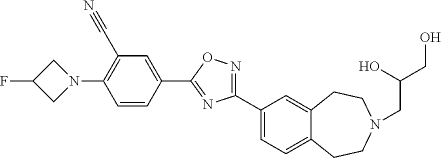

2-(3-Fluoro-1-azetidinyl)-5-[3-(2,3,4,5-tetrahydro-1H-3-benzazepin-7-yl)-1,2,4-oxadiazol-5-yl]benzonitrile (Preparation 108) (100 mg, 0.26 mmol), 2,3-dihydroxypropanal (35 mg, 0.39 mmol) and sodium triacetoxyborohydride (82 mg, 0.39 mmol) were combined with DCM (5 ml), and acetic acid (0.018 ml, 0.31 mmol) was added. The mixture was stirred at room temperature for 2 h. Further portions of 2,3-dihydroxypropanal (81 mg, 0.90 mmol) and sodium triacetoxyborohydride (190 mg, 0.90 mmol) were added, and the mixture was stirred overnight. Sodium triacetoxyborohydride (136 mg, 0.640 mmol) was added, and the mixture was stirred for ˜3 days. The reaction was partitioned between DCM (2×5 ml) and saturated sodium hydrogen carbonate solution (5 ml). The organic phases were combined, dried (hydrophobic frit) and reduced to dryness under a stream of nitrogen (some product lost in process). The residual solid was dissolved in DMSO (1 ml) and purified by MDAP (Method HpH) The solvent was evaporated under a stream of nitrogen to give 5-{3-[3-(2,3-dihydroxypropyl)-2,3,4,5-tetrahydro-1H-3-benzazepin-7-yl]-1,2,4-oxadiazol-5-yl}-2-(3-fluoro-1-azetidinyl)benzonitrile (5 mg).

LCMS (Method HpH): Retention time 1.08 min, MH⁺=464

### Example 105

### N-{2-[7-(5-{3-cyano-4-[(1-methylethyl)oxy]phenyl}-1,2,4-oxadiazol-3-yl)-1,2,4,5-tetrahydro-3H-3-benzazepin-3-yl]ethyl}-N′-ethylurea

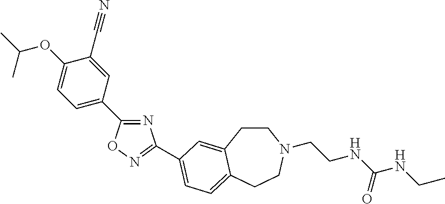

5-{3-[3-(2-Aminoethyl)-2,3,4,5-tetrahydro-1H-3-benzazepin-7-yl]-1,2,4-oxadiazol-5-yl}-2-[(1-methylethyl)oxy]benzonitrile hydrochloride (Preparation 111) (120 mg, 0.25 mmol) and triethylamine (0.102 ml, 0.73 mmol) were dissolved in DCM (3 ml) and stirred at room temperature. Isocyanatoethane (0.023 ml, 0.29 mmol) was added and the mixture stirred for 30 min. Water (10 ml) was added and the mixture was extracted using DCM (3×10 ml). The organic extracts were combined and dried (hydrophobic frit). The volatiles were removed under vacuum and the residue purified by flash chromatography (methanol/DCM, 0-5% gradient) to give N-{2-[7-(5-{3-cyano-4-[(1-methylethyl)oxy]phenyl}-1,2,4-oxadiazol-3-yl)-1,2,4,5-tetrahydro-3H-3-benzazepin-3-yl]ethyl}-N′-ethylurea as a colourless solid (78 mg).

LCMS (Method formate): Retention time 0.93 min, MH⁺=489

### Example 106

### 5-(3-{3-[(3-hydroxy-1-azetidinyl)acetyl]-2,3,4,5-tetrahydro-1H-3-benzazepin-7-yl}-1,2,4-oxadiazol-5-yl)-2-[(1-methylethyl)oxy]benzonitrile hydrochloride

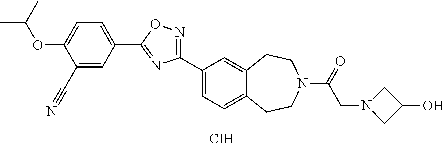

Acetonitrile (3 ml) was added to a mixture of 5-{3-[3-(bromoacetyl)-2,3,4,5-tetrahydro-1H-3-benzazepin-7-yl]-1,2,4-oxadiazol-5-yl}-2-[(1-methylethyl)oxy]benzonitrile (Preparation 102) (99 mg, 0.2 mmol), 3-azetidinol hydrochloride (44 mg, 0.40 mmol) and potassium carbonate (138 mg, 1.0 mmol) and the mixture stirred under nitrogen at 60° C. for −30 min. Further portions of 3-azetidinol hydrochloride (88 mg, 0.8 mmol) and potassium carbonate (220 mg) were added and heating continued for 1 h. The reaction was cooled to room temperature filtered and the residue washed with ethyl acetate. The filtrate and washings were reduced to dryness in vacuo, diluted with ethyl acetate and washed with saturated sodium hydrogen carbonate. The aqueous was extracted, the combined organic phases dried (MgSO₄) and concentrated in vacuo. The residue was purified by MDAP (Method HpH) and the isolated residue was dissolved in 1,4-dioxane (2 ml) and treated with hydrogen chloride in 1,4-dioxane (1 ml). The solvant was evaporated in vacuo and the residue triturated with diethyl ether to give 5-(3-{3-[(3-hydroxy-1-azetidinyl)acetyl]-2,3,4,5-tetrahydro-1H-3-benzazepin-7-yl}-1,2,4-oxadiazol-5-yl)-2-[(1-methylethyl)oxy]benzonitrile hydrochloride (16 mg, 15%) as a pale yellow solid.

LCMS (Method HpH): Retention time 1.10 min, MH⁺=488

### Example 107

### 5-[3-(3-glycyl-2,3,4,5-tetrahydro-1H-3-benzazepin-7-yl)-1,2,4-oxadiazol-5-yl]-2-(propyloxy)-3-pyridinecarbonitrile

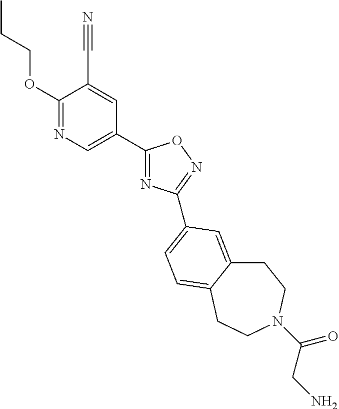

To 1,1-dimethylethyl [2-(7-{5-[5-cyano-6-(propyloxy)-3-pyridinyl]-1,2,4-oxadiazol-3-yl}-1,2,4,5-tetrahydro-3H-3-benzazepin-3-yl)-2-oxoethyl]carbamate (Preparation 115) (313 mg, 0.59 mmol) in DCM (2 ml) was added trifluoroacetic acid (200 μl, 2.6 mmol) and the reaction mixture stirred at room temperature for 5 h. The reaction was applied directly to an aminopropyl SPE (2 g) and the SPE eluted with methanol in DCM (10%). The appropriate fractions were combined and reduced to dryness under a stream of nitrogen. The sample was dissolved in DMSO (2×1 ml) and purified by MDAP (x2, Method HpH). The solvent was evaporated under a stream of nitrogen to give 5-[3-(3-glycyl-2,3,4,5-tetrahydro-1H-3-benzazepin-7-yl)-1,2,4-oxadiazol-5-yl]-2-(propyloxy)-3-pyridinecarbonitrile (72 mg).

LCMS (Method formate): Retention time 0.97 min, MH⁺=433

### Example 108

### 5-(3-{3-[2-hydroxy-1-(hydroxymethyl)ethyl]-2,3,4,5-tetrahydro-1H-3-benzazepin-7-yl}-1,2,4-oxadiazol-5-yl)-2-[(1-methylethyl)amino]-3-pyridinecarbonitrile hydrochloride

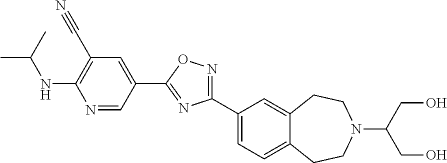

Sodium triacetoxyborohydride (2.12 g, 10 mmol) was added portionwise to a stirred solution of 2-[(1-methylethyl)amino]-5-[3-(2,3,4,5-tetrahydro-1H-3-benzazepin-7-yl)-1,2,4-oxadiazol-5-yl]-3-pyridinecarbonitrile hydrochloride (Preparation 120) (822 mg, 2 mmol) and 2,2-dimethyl-1,3-dioxan-5-one (716 μl, 6 mmol) in DCM (20 ml). The reaction mixture was stirred at room temperature overnight. Saturated sodium hydrogen carbonate (40 ml) was added and the mixture stirred for 15 min. The organic phase was separated. The aqueous phase was extracted with DCM (2×10 ml). The combined organics were dried and evaporated. The residue was dissolved in THF (5 ml) and the solution treated with 2M hydrochloric acid (2M, 5 ml). The reaction mixture was stirred at room temperature for 5 h. Saturated sodium hydrogen carbonate (20 ml) was added and the mixture stirred for 10 min. The organic phase was separated. The aqueous phase was extracted with DCM (3×10 ml). The combined organics were dried and evaporated. The residue was chromatographed (methanol/DCM, 0-6% gradient). The combined product fractions were treated with hydrogen chloride in diethyl ether (1M, 5 ml). The solvent was evaporated and the residue triturated with diethyl ether to give 5-(3-{3-[2-hydroxy-1-(hydroxymethyl)ethyl]-2,3,4,5-tetrahydro-1H-3-benzazepin-7-yl}-1,2,4-oxadiazol-5-yl)-2-[(1-methylethyl)amino]-3-pyridinecarbonitrile hydrochloride as a colourless solid (275 mg).

LCMS (Method formate): Retention time 0.79 min, MH⁺=449

### Example 109

### 5-(3-{3-[2-hydroxy-1-(hydroxymethyl)ethyl]-2,3,4,5-tetrahydro-1H-3-benzazepin-7-yl}-1,2,4-oxadiazol-5-yl)-2-(propyloxy)-3-pyridinecarbonitrile

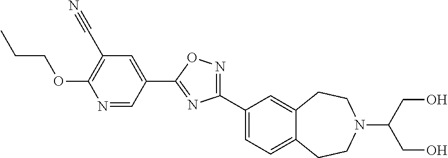

To 5-{3-[3-(2,2-dimethyl-1,3-dioxan-5-yl)-2,3,4,5-tetrahydro-1H-3-benzazepin-7-yl}-1,2,4-oxadiazol-5-yl}-2-(propyloxy)-3-pyridinecarbonitrile (Preparation 124) (180 mg, 0.37 mmol) in THF (3 ml) was added aqueous hydrochloric acid (2M, 3 ml, 99 mmol) and the reaction mixture stirred at room temperature for 3 h. The reaction was concentrated under a stream of nitrogen and the residue partitioned between DCM/ethyl acetate (1:1, 3×10 ml) and saturated aqueous sodium hydrogen carbonate solution (10 ml). The organics were combined, dried (hydrophobic frit) and reduced to dryness under a stream of nitrogen. The residue was dissolved in DMSO (1 ml) and purified by MDAP (Method HpH). The solvent was evaporated under a stream of nitrogen to give 5-(3-{3-[2-hydroxy-1-(hydroxymethyl)ethyl]-2,3,4,5-tetrahydro-1H-3-benzazepin-7-yl}-1,2,4-oxadiazol-5-yl)-2-(propyloxy)-3-pyridinecarbonitrile (82 mg).

LCMS (Method formate): Retention time 0.93 min, MH⁺=450

### Example 110

### 2-(ethyloxy)-5-(3-{3-[2-hydroxy-1-(hydroxymethyl)ethyl]-2,3,4,5-tetrahydro-1H-3-benzazepin-7-yl}-1,2,4-oxadiazol-5-yl)-3-pyridinecarbonitrile

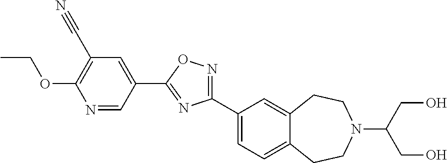

To 5-{3-[3-(2,2-dimethyl-1,3-dioxan-5-yl)-2,3,4,5-tetrahydro-1H-3-benzazepin-7-yl]-1,2,4-oxadiazol-5-yl}-2-(ethyloxy)-3-pyridinecarbonitrile (Preparation 125) (240 mg, 0.51 mmol) in THF (3 ml) was added aqueous hydrochloric acid (2M, 3 ml, 99 mmol) and the reaction mixture stirred at room temperature for 3 h. The reaction was concentrated under a stream of nitrogen and the residue partitioned between DCM/ethyl acetate (1:1, 3×10 ml) and saturated aqueous sodium hydrogen carbonate solution (10 ml). The organics were combined, dried (hydrophobic frit) and reduced to dryness under a stream of nitrogen. The residue was dissolved in DMSO (1 ml) and purified by MDAP (Method HpH). The solvent was evaporated under a stream of nitrogen to give 2-(ethyloxy)-5-(3-{3-[2-hydroxy-1-(hydroxymethyl)ethyl]-2,3,4,5-tetrahydro-1H-3-benzazepin-7-yl}-1,2,4-oxadiazol-5-yl)-3-pyridinecarbonitrile (38 mg).

LCMS (Method formate): Retention time 0.86 min, MH⁺=436

### Example 111

### 2-[7-(5-{3-cyano-4-[(1-methylethyl)oxy]phenyl}-1,2,4-oxadiazol-3-yl)-1,2,4,5-tetrahydro-3H-3-benzazepin-3-yl]-N-(2-hydroxyethyl)acetamide hydrochloride

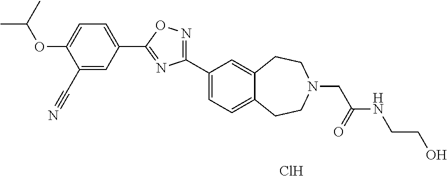

To a solution of [7-(5-{3-cyano-4-[(1-methylethyl)oxy]phenyl}-1,2,4-oxadiazol-3-yl)-1,2,4,5-tetrahydro-3H-3-benzazepin-3-yl]acetic acid (Preparation 89) (86 mg, 0.2 mmol) in DMF (2 ml) at room temperature were added HATU (76 mg, 0.20 mmol) then DIPEA (0.11 ml, 0.60 mmol) and after 5 min ethanolamine (0.018 ml, 0.30 mmol). The resulting solution was stirred at room temperature overnight. The solvent was removed, the residue partitioned between ethyl acetate and saturated sodium hydrogen carbonate. The aqueous was extracted, the combined organics washed with brine, dried (MgSO₄) and concentrated in vacuo. The residue was dissolved in 1,4-dioxane (3 ml) and treated with hydrogen chloride in 1,4-dioxane (4N). The solvent was removed, the residue triturated with diethyl ether and the solid isolated by filtration to give 2-[7-(5-{3-cyano-4-[(1-methylethyl)oxy]phenyl}-1,2,4-oxadiazol-3-yl)-1,2,4,5-tetrahydro-3H-3-benzazepin-3-yl]-N-(2-hydroxyethyl)acetamide hydrochloride as a white solid (43 mg, 42%).

LCMS (Method HpH): Retention time 1.15 min, MH⁺=476

### Example 112

### 5-[3-(3-glycyl-2,3,4,5-tetrahydro-1H-3-benzazepin-7-yl)-1,2,4-oxadiazol-5-yl]-2-[(1-methylethyl)amino]-3-pyridinecarbonitrile

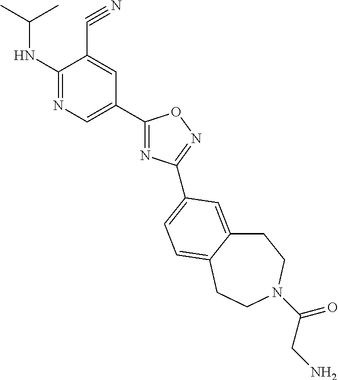

To 1,1-dimethylethyl {2-[7-(5-{5-cyano-6-[(1-methylethyl)amino]-3-pyridinyl}-1,2,4-oxadiazol-3-yl)-1,2,4,5-tetrahydro-3H-3-benzazepin-3-yl]-2-oxoethyl}carbamate (Preparation 128) (315 mg, 0.59 mmol) in DCM (2 ml) was added trifluoroacetic acid (200 μl, 2.6 mmol) and the reaction mixture stirred at room temperature overnight. The reaction mixture was applied an aminopropyl SPE (2 g) and the SPE eluted with methanol in DCM (10%). The appropriate fractions were combined and reduced to dryness under a stream of nitrogen. The residue was dissolved in DMSO (2×1 ml) and purified by MDAP (Method HpH). The solvents were evaporated under a stream of nitrogen to give 5-[3-(3-glycyl-2,3,4,5-tetrahydro-1H-3-benzazepin-7-yl)-1,2,4-oxadiazol-5-yl]-2-[(1-methylethyl)amino]-3-pyridinecarbonitrile (40 mg).

LCMS (Method formate): Retention time 0.91 min, MH⁺=432

### Example 113

### 2-(ethyloxy)-5-[3-(3-glycyl-2,3,4,5-tetrahydro-1H-3-benzazepin-7-yl)-1,2,4-oxadiazol-5-yl]-3-pyridinecarbonitrile

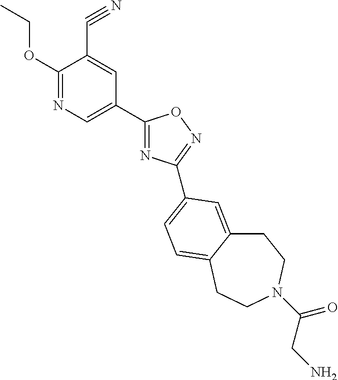

To 1,1-dimethylethyl [2-(7-{5-[5-cyano-6-(ethyloxy)-3-pyridinyl]-1,2,4-oxadiazol-3-yl}-1,2,4,5-tetrahydro-3H-3-benzazepin-3-yl)-2-oxoethyl]carbamate (Preparation 129) (238 mg, 0.46 mmol) in DCM (2 ml) was added trifluoroacetic acid (200 μl, 2.6 mmol) and the reaction mixture stirred at room temperature for 5 h. The reaction mixture was applied an aminopropyl SPE (2 g) and the SPE eluted using methanol in DCM (10%). The appropriate fractions were combined and reduced to dryness under a stream of nitrogen. The residue was dissolved in DMSO (2×1 ml) and purified by MDAP (x2, Method HpH). The solvent was evaporated under a stream of nitrogen to give 2-(ethyloxy)-5-[3-(3-glycyl-2,3,4,5-tetrahydro-1H-3-benzazepin-7-yl)-1,2,4-oxadiazol-5-yl]-3-pyridinecarbonitrile (40 mg).

LCMS (Method formate): Retention time 0.90 min, MH⁺=419

### Example 114

### 5-(3-{3-[(2S)-2,3-dihydroxypropyl]-2,3,4,5-tetrahydro-1H-3-benzazepin-7-yl}-1,2,4-oxadiazol-5-yl)-2-[(1-methylethyl)amino]-3-pyridinecarbonitrile

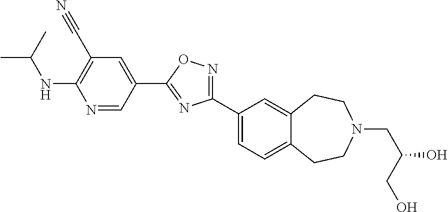

To 2-[(1-methylethyl)amino]-5-[3-(2,3,4,5-tetrahydro-1H-3-benzazepin-7-yl)-1,2,4-oxadiazol-5-yl]-3-pyridinecarbonitrile (Preparation 120) (100 mg, 0.27 mmol) in THF (1.5 ml) and DCM (1.5 ml) was added D-glyceraldehyde (120 mg, 1.3 mmol) and acetic acid (0.016 ml, 0.28 mmol). The reaction mixture was stirred for 30 min at room temperature. Sodium triacetoxyborohydride (283 mg, 1.3 mmol) was added and the reaction mixture stirred at room temperature overnight. A further portion of D-glyceraldehyde (120 mg, 1.3 mmol) was added and stirring was continued at room temperature for 24 h. Sodium triacetoxyborohydride (283 mg, 1.3 mmol) was added and stirring was continued for 24 h. The reaction was quenched with water (10 ml) and extracted with DCM (3×10 ml). The organics were combined, dried (hydrophobic frit) and reduced to dryness under a stream of nitrogen. The residue was dissolved in DMSO (1 ml) and purified by MDAP (Method HpH). The solvent was evaporated under a stream of nitrogen to give 5-(3-{3-[(2S)-2,3-dihydroxypropyl]-2,3,4,5-tetrahydro-1H-3-benzazepin-7-yl}-1,2,4-oxadiazol-5-yl)-2-[(1-methylethyl)amino]-3-pyridinecarbonitrile (30 mg).

LCMS (Method formate): Retention time 0.86 min, MH⁺=449

### Example 115

### 5-(3-{3-[(2S)-2,3-dihydroxypropyl]-2,3,4,5-tetrahydro-1H-3-benzazepin-7-yl}-1,2,4-oxadiazol-5-yl)-2-(propyloxy)-3-pyridinecarbonitrile

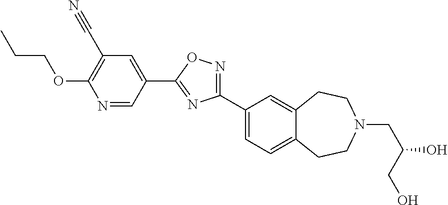

To 2-(propyloxy)-5-[3-(2,3,4,5-tetrahydro-1H-3-benzazepin-7-yl)-1,2,4-oxadiazol-5-yl]-3-pyridinecarbonitrile (Preparation 116) (100 mg, 0.27 mmol) in THF (1.5 ml) and DCM (1.5 ml) was added D-glyceraldehyde (120 mg, 1.3 mmol) and acetic acid (0.016 ml, 0.28 mmol) and the reaction mixture stirred at room temperature for 30 min. Sodium triacetoxyborohydride (282 mg, 1.3 mmol) was added and the reaction mixture stirred at room temperature overnight. Further D-glyceraldehyde (120 mg, 1.3 mmol) was added and the reaction mixture stirred for 24 h, sodium triacetoxyborohydride (282 mg, 1.3 mmol) was added and the reaction mixture stirred for a further 24 h. A few drops of methanol were added and the reaction mixture stirred at room temperature for 3 h, sodium triacetoxyborohydride (282 mg, 1.3 mmol) was then added and the reaction mixture stirred at room temperature for 2 h. The reaction was quenched with water (10 ml) and the mixture extracted with DCM (3×10 ml). The organics were combined, dried (hydrophobic frit) and reduced to dryness under a stream of nitrogen. The residue was dissolved in DMSO (1 ml) and purified by MDAP (Method HpH). The solvent was evaporated under a stream of nitrogen to give 5-(3-{3-[(2S)-2,3-dihydroxypropyl]-2,3,4,5-tetrahydro-1H-3-benzazepin-7-yl}-1,2,4-oxadiazol-5-yl)-2-(propyloxy)-3-pyridinecarbonitrile (61 mg).

LCMS (Method formate): Retention time 0.93 min, MH⁺=450

### Example 116

### 5-(3-{34[(2S)-2,3-dihydroxypropyl]-2,3,4,5-tetrahydro-1H-3-benzazepin-7-yl}-1,2,4-oxadiazol-5-yl)-2-(ethyloxy)-3-pyridinecarbonitrile

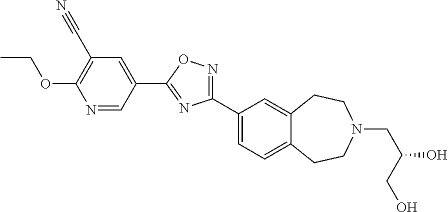

To 2-(ethyloxy)-5-[3-(2,3,4,5-tetrahydro-1H-3-benzazepin-7-yl)-1,2,4-oxadiazol-5-yl]-3-pyridinecarbonitrile (Preparation 126) (100 mg, 0.28 mmol) in THF (1.5 ml) and DCM (1.5 ml) was added D-glyceraldehyde (125 mg, 1.4 mmol) and acetic acid (0.017 ml, 0.29 mmol) and the reaction mixture stirred at room temperature for 30 min. Sodium triacetoxyborohydride (293 mg, 1.4 mmol) was added and the reaction mixture stirred at room temperature overnight. Further D-glyceraldehyde (125 mg, 1.4 mmol) was added and the reaction mixture stirred at room temperature for 24 h, sodium triacetoxyborohydride (293 mg, 1.4 mmol) was added and the reaction mixture stirred at room temperature for 24 h. A few drops of methanol were added and the reaction mixture stirred at room temperature for 3 h, sodium triacetoxyborohydride (293 mg, 1.4 mmol) was added and the reaction mixture stirred at room temperature for 2 h. The reaction was quenched with water (10 ml) and the mixture extracted with DCM (3×10 ml). The organic phases were combined, dried (hydrophobic frit) and reduced to dryness under a stream of nitrogen. The residue was dissolved in DMSO (1 ml) and purified by MDAP (Method HpH). The solvent was evaporated under a stream of nitrogen to give 5-(3-{3-[(2S)-2,3-dihydroxypropyl]-2,3,4,5-tetrahydro-1H-3-benzazepin-7-yl}-1,2,4-oxadiazol-5-yl)-2-(ethyloxy)-3-pyridinecarbonitrile (30 mg).

LCMS (Method formate): Retention time 0.85 min, MH⁺=436

### Example 117

### 5-{3-[3-(2-methylalanyl)-2,3,4,5-tetrahydro-1H-3-benzazepin-7-yl]-1,2,4-oxadiazol-5-yl}-2-[(1-methylethyl)oxy]benzonitrile

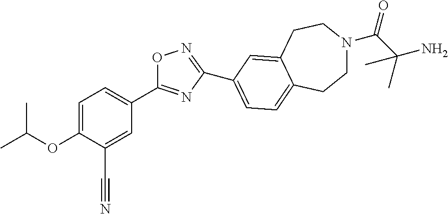

To 1,1-dimethylethyl {2-[7-(5-{3-cyano-4-[(1-methylethyl)oxy]phenyl}-1,2,4-oxadiazol-3-yl)-1,2,4,5-tetrahydro-3H-3-benzazepin-3-yl]-1,1-dimethyl-2-oxoethyl}carbamate (Preparation 130) (127 mg, 0.23 mmol) in DCM (2 ml) was added trifluoroacetic acid (200 μl, 2.6 mmol) and the reaction mixture stirred at room temperature overnight. The reaction mixture was applied to an aminopropyl SPE (2 g) and the SPE eluted with methanol in DCM (10%). The appropriate fractions were combined and reduced to dryness under a stream of nitrogen. The residue was dissolved in DMSO (2×1 ml) and purified by MDAP (x2, Method HpH). The solvent was evaporated under a stream of nitrogen to give 5-{3-[3-(2-methylalanyl)-2,3,4,5-tetrahydro-1H-3-benzazepin-7-yl]-1,2,4-oxadiazol-5-yl}-2-[(1-methylethyl)oxy]benzonitrile (40 mg).

LCMS (Method formate): Retention time 0.97 min, MH⁺=460

### Example 118

### 2-[6-(5-{3-cyano-4-[(1-methylethyl)oxy]phenyl}-1,2,4-oxadiazol-3-yl)-5-methyl-3,4-dihydro-2(1H)-isoquinolinyl]-N-(1R)-2-hydroxy-1-methylethyl]acetamide hydrochloride

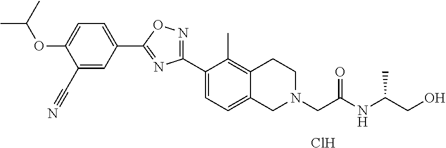

[6-(5-{3-Cyano-4-[(1-methylethyl)oxy]phenyl}-1,2,4-oxadiazol-3-yl)-5-methyl-3,4-dihydro-2(1H)-isoquinolinyl]acetic acid (Preparation 29) (86 mg, 0.2 mmol) was suspended in DMF (3 ml), warmed and then allowed to cool to room temperature. To the suspension was added HATU (76 mg, 0.20 mmol) and DIPEA (0.21 ml, 1.2 mmol). After 5 min, (2R)-2-amino-1-propanol (23 mg, 0.30 mmol) was added and the mixture stirred for 4 h. Acetonitrile (1 ml), DMSO (1 ml) and NMP (2 ml) were added followed by HATU (76 mg, 0.20 mmol) and stirring continued. DIPEA (0.11 ml, 0.60 mmol) and (2R)-2-amino-1-propanol (23 mg, 0.30 mmol) were added to the mixture and stirring continued for 30 min. The solution was diluted with saturated sodium hydrogen carbonate and the aqueous phase extracted with ethyl acetate (x3). The combined organic phases were washed with saturated sodium hydrogen carbonate, dried (MgSO₄) and concentrated in vacuo. The residue was purified by MDAP (Method HpH). The residue was dissolved in 1,4-dioxane (2 ml), treated with hydrogen chloride in 1,4-dioxane (4N, 1 ml) and the mixture concentrated in vacuo. The residue was triturated with diethyl ether and the solid isolated by filtration to give 2-[6-(5-{3-cyano-4-[(1-methylethyl)oxy]phenyl}-1,2,4-oxadiazol-3-yl)-5-methyl-3,4-dihydro-2(1H)-isoquinolinyl]-N-(1R)-2-hydroxy-1-methylethyl]acetamide hydrochloride as a pale yellow solid (28 mg, 27%).

LCMS (Method HpH): Retention time 1.18 min, MH⁺=490

### Example 119

### 2-[6-(5-{3-cyano-4-[(1-methylethyl)oxy]phenyl}-1,2,4-oxadiazol-3-yl)-5-methyl-3,4-dihydro-2(1H)-isoquinolinyl]-N-[(1S)-2-hydroxy-1-methylethyl]acetamide hydrochloride

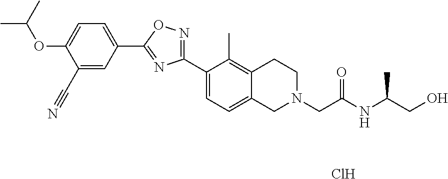

Finely crushed [6-(5-{3-cyano-4-[(1-methylethyl)oxy]phenyl}-1,2,4-oxadiazol-3-yl)-5-methyl-3,4-dihydro-2(1H)-isoquinolinyl]acetic acid (Preparation 29) (86 mg, 0.2 mmol) was suspended in DMF (2 ml) and NMP (2.0 ml). The suspension was warmed and then cooled to room temperature. DIPEA (0.11 ml, 0.6 mmol) was added, then HATU (76 mg, 0.20 mmol) and after 5 min (2S)-2-amino-1-propanol (23 mg, 0.30 mmol). The resulting deep yellow mixture was stirred for 1 h. DIPEA (0.11 ml, 0.6 mmol) and HATU (76 mg, 0.20 mmol) were added, the mixture stirred for 5 min, treated with (2S)-2-amino-1-propanol (23 mg, 0.30 mmol) and stirred for a further ˜20 min. The reaction was partitioned between saturated sodium hydrogen carbonate and ethyl acetate, the aqueous extracted (x2) and the combined organic phases washed with saturated sodium hydrogen carbonate. The organic solution was dried (MgSO₄) and concentrated in vacuo to give 2-[6-(5-{3-cyano-4-[(1-methylethyl)oxy]phenyl}-1,2,4-oxadiazol-3-yl)-5-methyl-3,4-dihydro-2(1H)-isoquinolinyl]-N-[(1S)-2-hydroxy-1-methylethyl]acetamide hydrochloride as a white solid (46 mg).

LCMS (Method HpH): Retention time 1.19 min, MH⁺=490

### Example 120

### 2-[6-(5-{3-cyano-4-[(1-methylethyl)oxy]phenyl}-1,2,4-oxadiazol-3-yl)-5-methyl-3,4-dihydro-2(1H)-isoquinolinyl]-N-[(2R)-2-hydroxypropyl]acetamide hydrochloride

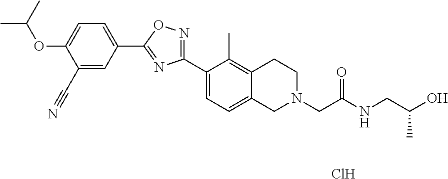

Finely crushed [6-(5-{3-cyano-4-[(1-methylethyl)oxy]phenyl}-1,2,4-oxadiazol-3-yl)-5-methyl-3,4-dihydro-2(1H)-isoquinolinyl]acetic acid (Preparation 29) (86 mg, 0.2 mmol) was suspended in DMF (2 ml) and NMP (2.0 ml). The suspension was warmed and then cooled to room temperature. DIPEA (0.11 ml, 0.6 mmol) was added then HATU (0.076 g, 0.2 mmol) and after 5 min (2R)-1-amino-2-propanol (0.023 g, 0.3 mmol). The resulting deep yellow mixture was stirred for 1 h. DIPEA (0.11 ml, 0.6 mmol) and HATU (0.076 g, 0.20 mmol) were added. A further portion of HATU (200 mg) was added and stirring continued for 20 min. The reaction was partitioned between ethyl acetate and saturated sodium hydrogen carbonate, the aqueous phase extracted (x2) and the combined organic phases washed with saturated sodium hydrogen carbonate/brine. The organic solution was dried (MgSO₄) and concentrated in vacuo. Purification of the residue by MDAP (Method HpH) gave a residue which was dissolved in 1,4-dioxane (3 ml) and treated with hydrogen chloride in 1,4-dioxane (4N, 1 ml). The mixture was concentrated in vacuo and residue coevaporated with diethyl ether and triturated with diethyl ether. The solid was isolated by filtration to give 2-[6-(5-{3-cyano-4-[(1-methylethyl)oxy]phenyl}-1,2,4-oxadiazol-3-yl)-5-methyl-3,4-dihydro-2(1H)-isoquinolinyl]-N-[(2R)-2-hydroxypropyl]acetamide hydrochloride as a white solid.

LCMS (Method HpH): Retention time 1.18 min, MH⁺=490

### Example 121

### 2-[6-(5-{3-cyano-4-[(1-methylethyl)oxy]phenyl}-1,2,4-oxadiazol-3-yl)-5-methyl-3,4-dihydro-2(1H)-isoquinolinyl]-N-[(2S)-2-hydroxypropyl]acetamide hydrochloride

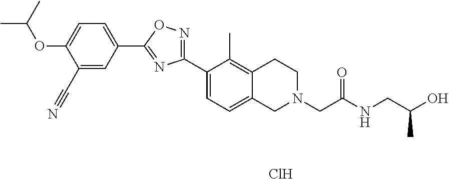

Finely crushed [6-(5-{3-cyano-4-[(1-methylethyl)oxy]phenyl}-1,2,4-oxadiazol-3-yl)-5-methyl-3,4-dihydro-2(1H)-isoquinolinyl]acetic acid (Preparation 29) (86 mg, 0.2 mmol) was suspended in DMF (2 ml) and NMP (2.0 ml). The suspension was warmed and then cooled to room temperature. DIPEA (0.11 ml, 0.6 mmol) was added, then HATU (76 mg, 0.20 mmol) and after 5 min (2S)-1-amino-2-propanol (15 mg, 0.20 mmol). The resulting deep yellow mixture was stirred for 1 h. DIPEA (0.11 ml, 0.6 mmol) and HATU (76 mg, 0.20 mmol) were added. A further portion of HATU (200 mg) was added and stirring continued for 20 min. The reaction was partitioned between ethyl acetate and saturated sodium hydrogen carbonate, the aqueous phase extracted (x2) and the combined organic phases washed with saturated sodium hydrogen carbonate/brine. The organic solution was dried (MgSO₄) and concentrated in vacuo. Purification of the residue by MDAP (Method HpH) gave a residue which was dissolved in 1,4-dioxane (3 ml) and treated with hydrogen chloride in 1,4-dioxane (4N, 1 ml). The mixture was concentrated in vacuo and residue coevaporated with diethyl ether and triturated with diethyl ether. The solid was isolated by filtration to give 2-[6-(5-{3-cyano-4-[(1-methylethyl)oxy]phenyl}-1,2,4-oxadiazol-3-yl)-5-methyl-3,4-dihydro-2(1H)-isoquinolinyl]-N-[(2S)-2-hydroxypropyl]acetamide hydrochloride (32 mg, 30%) as a pale yellow solid.

LCMS (Method HpH): Retention time 1.17 min, MH⁺=490

### Example 122

### 2-[6-(5-{3-cyano-4-[(1-methylethyl)oxy]phenyl}-1,2,4-oxadiazol-3-yl)-5-methyl-3,4-dihydro-2(1H)-isoquinolinyl]-N-(2-hydroxyethyl)acetamide hydrochloride

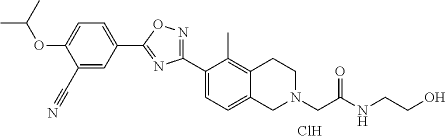

Finely crushed [6-(5-{3-cyano-4-[(1-methylethyl)oxy]phenyl}-1,2,4-oxadiazol-3-yl)-5-methyl-3,4-dihydro-2(1H)-isoquinolinyl]acetic acid (Preparation 29) (86 mg, 0.2 mmol) was suspended in DMF (2 ml) and NMP (2.0 ml). The suspension was warmed and then cooled to room temperature. DIPEA (0.11 ml, 0.6 mmol) was added, then HATU (76 mg, 0.20 mmol) and after 5 min ethanolamine (0.018 ml, 0.30 mmol). The resulting deep yellow mixture was stirred for 1 h. DIPEA (0.11 ml, 0.6 mmol) and HATU (76 mg, 0.20 mmol) were added. A further portion of HATU (200 mg) was added and stirring continued for 20 min. The reaction was partitioned between ethyl acetate and saturated sodium hydrogen carbonate, the aqueous phase extracted (x2) and the combined organic phases washed with saturated sodium hydrogen carbonate/brine. The organic solution was dried (MgSO₄) and concentrated in vacuo. Purification of the residue by MDAP (Method HpH) gave a residue which was dissolved in 1,4-dioxane (3 ml) and treated with hydrogen chloride in 1,4-dioxane (4N, 1 ml). The mixture was concentrated in vacuo and residue coevaporated with diethyl ether and triturated with diethyl ether. The solid was isolated by filtration to give 2-[6-(5-{3-cyano-4-[(1-methylethyl)oxy]phenyl}-1,2,4-oxadiazol-3-yl)-5-methyl-3,4-dihydro-2(1H)-isoquinolinyl]-N-(2-hydroxyethyl)acetamide hydrochloride (22 mg, 21%) as a white solid.

LCMS (Method HpH): Retention time 1.15 min, MH⁺=476

### Example 123

### 5-(3-{2-[2-(3-hydroxy-1-azetidinyl)-2-oxoethyl]-5-methyl-1,2,3,4-tetrahydro-6-isoquinolinyl}-1,2,4-oxadiazol-5-yl)-2-[(1-methylethyl)oxy]benzonitrile hydrochloride

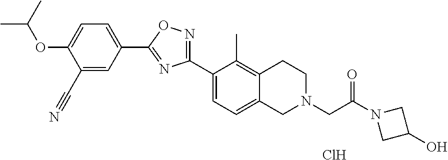

Finely crushed [6-(5-{3-cyano-4-[(1-methylethyl)oxy]phenyl}-1,2,4-oxadiazol-3-yl)-5-methyl-3,4-dihydro-2(1H)-isoquinolinyl]acetic acid (Preparation 29) (86 mg, 0.2 mmol) was suspended in DMF (2 ml) and NMP (2.0 ml). The suspension was warmed and then cooled to room temperature. DIPEA (0.11 ml, 0.6 mmol) was added, then HATU (76 mg, 0.20 mmol) and after 5 min 3-azetidinol hydrochloride (33 mg, 0.30 mmol). The resulting deep yellow mixture was stirred for 1 h. DIPEA (0.11 ml, 0.6 mmol) and HATU (76 mg, 0.20 mmol) were added and after 5 min, 3-azetidinol hydrochloride (33 mg, 0.30 mmol). A further portion of HATU (30 mg) was added and stirring continued for 40 min. The reaction was partitioned between ethyl acetate and saturated sodium hydrogen carbonate, the aqueous phase extracted (x2) and the combined organic phases washed with saturated sodium hydrogen carbonate/brine. The organic solution was dried (MgSO₄) and concentrated in vacuo. Purification of the residue by MDAP (Method HpH) gave a residue which was dissolved in 1,4-dioxane (3 ml) and treated with hydrogen chloride in 1,4-dioxane (4N, 1 ml). The mixture was concentrated in vacuo and residue coevaporated with diethyl ether and triturated with diethyl ether. The solid was isolated by filtration to give 5-(3-{2-[2-(3-hydroxy-1-azetidinyl)-2-oxoethyl]-5-methyl-1,2,3,4-tetrahydro-6-isoquinolinyl}-1,2,4-oxadiazol-5-yl)-2-[(1-methylethyl)oxy]benzonitrile hydrochloride (41 mg, 39%) as a white solid.

LCMS (Method HpH): Retention time 1.11 min, MH⁺=488

### Example 124

### 2-[6-(5-{3-cyano-4-[(1-methylethyl)oxy]phenyl}-1,2,4-oxadiazol-3-yl)-5-methyl-3,4-dihydro-2(1H)-isoquinolinyl]-N-[(2S)-2,3-dihydroxypropyl]acetamide hydrochloride

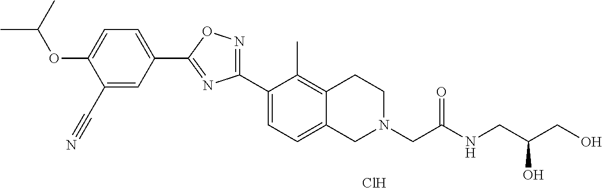

Finely crushed [6-(5-{3-cyano-4-[(1-methylethyl)oxy]phenyl}-1,2,4-oxadiazol-3-yl)-5-methyl-3,4-dihydro-2(1H)-isoquinolinyl]acetic acid (Preparation 29) (86 mg, 0.2 mmol) was suspended in DMF (2 ml) and NMP (2.0 ml). The suspension was warmed and then cooled to room temperature. DIPEA (0.11 ml, 0.6 mmol) was added, then HATU (76 mg, 0.20 mmol) and after 5 min (2S)-3-amino-1,2-propanediol (27 mg, 0.3 mmol). The resulting deep yellow mixture was stirred for 1 h. DIPEA (0.11 ml, 0.6 mmol) and HATU (76 mg, 0.2 mmol) were added and after 5 min (2S)-3-amino-1,2-propanediol (27 mg, 0.30 mmol). A further portion of HATU (30 mg) was added and stirring continued for 40 min. The reaction was partitioned between ethyl acetate and saturated sodium hydrogen carbonate, the aqueous phase extracted (x2) and the combined organic phases washed with saturated sodium hydrogen carbonate/brine. The organic solution was dried (MgSO₄) and concentrated in vacuo. Purification of the residue by MDAP (Method HpH) gave a residue which was dissolved in 1,4-dioxane (3 ml) and treated with hydrogen chloride in 1,4-dioxane (4N, 1 ml). The mixture was concentrated in vacuo and residue coevaporated with diethyl ether and triturated with diethyl ether. The solid was isolated by filtration to give 2-[6-(5-{3-cyano-4-[(1-methylethyl)oxy]phenyl}-1,2,4-oxadiazol-3-yl)-5-methyl-3,4-dihydro-2(1H)-isoquinolinyl]-N-[(2S)-2,3-dihydroxypropyl]acetamide hydrochloride (53 mg, 49%) as a white solid.

LCMS (Method HpH): Retention time 1.10 min, MH⁺=506

### Example 125

### 2-[6-(5-{3-cyano-4-[(1-methylethyl)oxy]phenyl}-1,2,4-oxadiazol-3-yl)-5-methyl-3,4-dihydro-2(1H)-isoquinolinyl]-N-[2-hydroxy-1-(hydroxymethyl)ethyl]acetamide hydrochloride

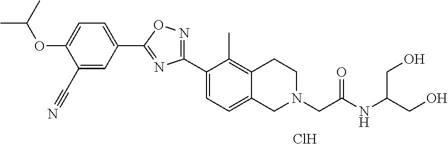

Finely crushed [6-(5-{3-cyano-4-[(1-methylethyl)oxy]phenyl}-1,2,4-oxadiazol-3-yl)-5-methyl-3,4-dihydro-2(1H)-isoquinolinyl]acetic acid (Preparation 29) (86 mg, 0.2 mmol) was suspended in DMF (2 ml) and NMP (2.0 ml). The suspension was warmed and then cooled to room temperature. DIPEA (0.11 ml, 0.6 mmol) was added, then HATU (76 mg, 0.20 mmol) and after 5 min 2-amino-1,3-propanediol hydrochloride (38 mg, 0.3 mmol). The resulting deep yellow mixture was stirred for 1 h. DIPEA (0.11 ml, 0.6 mmol) and HATU (76 mg, 0.2 mmol) were added and after 5 min 2-amino-1,3-propanediol hydrochloride (38 mg, 0.30 mmol). A further portion of HATU (30 mg) was added and stirring continued for 40 min. The reaction was partitioned between ethyl acetate and saturated sodium hydrogen carbonate, the aqueous phase extracted (x2) and the combined organic phases washed with saturated sodium hydrogen carbonate/brine. The organic solution was dried (MgSO₄) and concentrated in vacuo. Purification of the residue by MDAP (Method HpH) gave a residue which was dissolved in 1,4-dioxane (3 ml) and treated with hydrogen chloride in 1,4-dioxane (4N, 1 ml). The mixture was concentrated in vacuo and residue coevaporated with diethyl ether and triturated with diethyl ether. The solid was isolated by filtration to give 2-[6-(5-{3-cyano-4-[(1-methylethyl)oxy]phenyl}-1,2,4-oxadiazol-3-yl)-5-methyl-3,4-dihydro-2(1H)-isoquinolinyl]-N-[2-hydroxy-1-(hydroxymethyl)ethyl)acetamide hydrochloride (43 mg, 40%) as a white solid.

LCMS (Method HpH): Retention time 1.08 min, MH⁺=506

### Example 126

### 5-[3-(2-{N-[(2S)-2,3-dihydroxypropyl]glycyl}-5-methyl-1,2,3,4-tetrahydro-6-isoquinolinyl)-1,2,4-oxadiazol-5-yl]-2-[(1-methylethyl)oxy]benzonitrile hydrochloride

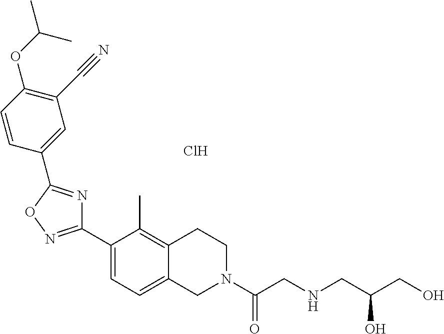

S-3-Amino-1,2-propanediol (92 mg, 1 mmol) was added to a stirred mixture of 5-{3-[2-(bromoacetyl)-5-methyl-1,2,3,4-tetrahydro-6-isoquinolinyl]-1,2,4-oxadiazol-5-yl}-2-[(1-methylethyl)oxy]benzonitrile (Preparation 131) (100 mg, 0.2 mmol) and potassium carbonate (70 mg, 0.5 mmol) in acetonitrile (3 ml). The reaction mixture was stirred at 50° C. for 1 h. The reaction mixture was filtered. The solvent was evaporated from the filtrate and the residue chromatographed (methanol/DCM, 5-15%). The product was dissolved in methanol (2 ml) and treated with hydrogen chloride in diethyl ether (1M, 0.5 ml). The solvent was evaporated and the residue triturated with diethyl ether to give 5-[3-(2-{N-[(2S)-2,3-dihydroxypropyl]glycyl}-5-methyl-1,2,3,4-tetrahydro-6-isoquinolinyl)-1,2,4-oxadiazol-5-yl]-2-[(1-methylethyl)oxy]benzonitrile hydrochloride as a pale yellow solid (32 mg).

LCMS (Method formate): Retention time 0.93 min, MH⁺=506

### Example 127

### 5-[3-(2-{N-[2-hydroxy-1-(hydroxymethyl)ethyl]glycyl}-5-methyl-1,2,3,4-tetrahydro-6-isoquinolinyl)-1,2,4-oxadiazol-5-yl]-2-[(1-methylethyl)oxy]benzonitrile hydrochloride

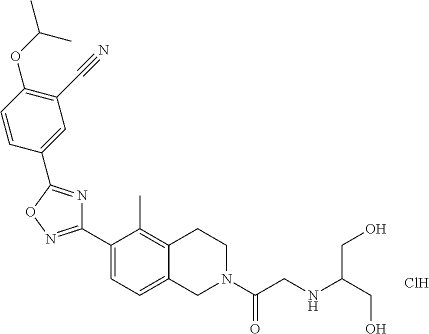

Serinol hydrochloride (129 mg, 1 mmol) was added to a stirred mixture of 5-{3-[2-(bromoacetyl)-5-methyl-1,2,3,4-tetrahydro-6-isoquinolinyl]-1,2,4-oxadiazol-5-yl}-2-[(1-methylethyl)oxy]benzonitrile (Preparation 131) (100 mg, 0.2 mmol) and potassium carbonate (70 mg, 0.5 mmol) in acetonitrile (3 ml). The reaction mixture was stirred at room temperature for 3 h. The reaction mixture was filtered and the solvent evaporated from the filtrate. The residue was chromatographed (methanol/DCM, 5-10%). The product was dissolved in DCM (5 ml) and treated with hydrogen chloride in diethyl ether (1 M, 0.3 ml). The solvent was evaporated and the residue triturated with diethyl ether to give 5-[3-(2-{N-[2-hydroxy-1-(hydroxymethyl)ethyl)glycyl}-5-methyl-1,2,3,4-tetrahydro-6-isoquinolinyl)-1,2,4-oxadiazol-5-yl]-2-[(1-methylethyl)oxy]benzonitrile hydrochloride as an off-white solid (21 mg).

LCMS (Method formate): Retention time 0.91 min, MH⁺=506

### Example 128

formic acid-5-[3-(2-{N-(1R)-2-hydroxy-1-methylethyl]glycyl}-5-methyl-1,2,3,4-tetrahydro-6-isoquinolinyl)-1,2,4-oxadiazol-5-yl]-2-[(1-methylethyl)oxy]benzonitrile (1:1)

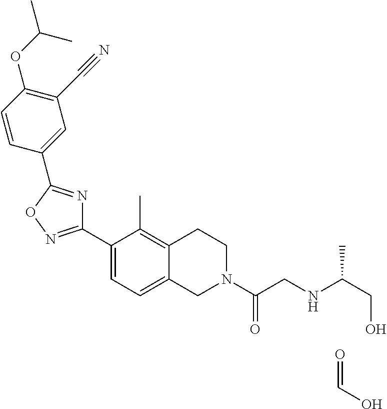

R-2-Amino-1-propanol (76 mg, 1 mmol) was added to a stirred mixture of 5-{3-[2-(bromoacetyl)-5-methyl-1,2,3,4-tetrahydro-6-isoquinolinyl]-1,2,4-oxadiazol-5-yl}-2-[(1-methylethyl)oxy]benzonitrile (Preparation 131) (100 mg, 0.2 mmol) and potassium carbonate (70 mg, 0.5 mmol) in acetonitrile (3 ml). The reaction mixture was stirred at room temperature for 3 h. The reaction mixture was filtered and the solvent evaporated from the filtrate. The residue was chromatographed (methanol/DCM, 5-10%). The residue was further purified by MDAP (Method formate). The solvent was evaporated and the residue triturated with diethyl ether to give formic acid-5-[3-(2-{N-(1R)-2-hydroxy-1-methylethyl]glycyl}-5-methyl-1,2,3,4-tetrahydro-6-isoquinolinyl)-1,2,4-oxadiazol-5-yl]-2-[(1-methylethyl)oxy]benzonitrile (1:1) as an off-white solid (21 mg).

LCMS (Method formate): Retention time 0.96 min, MH⁺=490

### Example 129

### 5-[3-(2-{N-[(2R)-2-hydroxypropyl]glycyl}-5-methyl-1,2,3,4-tetrahydro-6-isoquinolinyl)-1,2,4-oxadiazol-5-yl]-2-[(1-methylethyl)oxy]benzonitrile hydrochloride

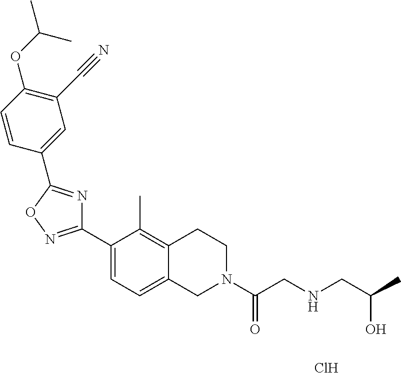

R-1-Amino-2-propanol (76 mg, 1 mmol) was added to a stirred mixture of 5-{3-[2-(bromoacetyl)-5-methyl-1,2,3,4-tetrahydro-6-isoquinolinyl]-1,2,4-oxadiazol-5-yl}-2-[(1-methylethyl)oxy]benzonitrile (Preparation 131) (100 mg, 0.2 mmol) and potassium carbonate (70 mg, 0.5 mmol) in acetonitrile (3 ml). The reaction mixture was stirred at room temperature for 3 h. The reaction mixture was filtered and the solvent evaporated from the filtrate. The residue was chromatographed (methanol/DCM, 5-10%). The product was dissolved in DCM (5 ml) and treated with hydrogen chloride in diethyl ether (1M, 0.3 ml). The solvent was evaporated and the residue triturated with diethyl ether to give 5-[3-(2-{N-[(2R)-2-hydroxypropyl]glycyl}-5-methyl-1,2,3,4-tetrahydro-6-isoquinolinyl)-1,2,4-oxadiazol-5-yl]-2-[(1-methylethyl)oxy]benzonitrile hydrochloride as an off-white solid (21 mg).

LCMS (Method formate): Retention time 0.92 min, MH⁺=490

### Example 130

### 5-[3-(2-{N-[(1S)-2-hydroxy-1-methylethyl]glycyl}-5-methyl-1,2,3,4-tetrahydro-6-isoquinolinyl)-1,2,4-oxadiazol-5-yl]-2-[(1-methylethyl)oxy]benzonitrile

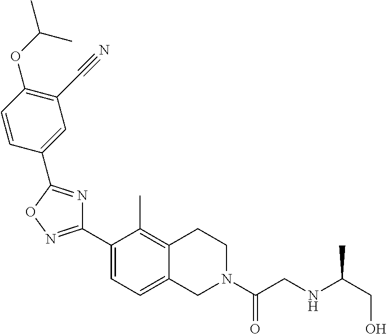

S-2-amino-1-propanol (L-alaninol) (76 mg, 1 mmol) was added to a stirred mixture of 5-{3-[2-(bromoacetyl)-5-methyl-1,2,3,4-tetrahydro-6-isoquinolinyl]-1,2,4-oxadiazol-5-yl}-2-[(1-methylethyl)oxy]benzonitrile (Preparation 131) (100 mg, 0.2 mmol) and potassium carbonate (70 mg, 0.5 mmol) in acetonitrile (3 ml). The reaction mixture was stirred at room temperature for 3 h. The reaction mixture was filtered and the solvent evaporated from the filtrate. The residue was chromatographed (methanol/DCM, 5-10%). The residue was further purified by MDAP (Method formate). The solvent was evaporated and the residue triturated with diethyl ether to give formic acid—5-[3-(2-{N-[(1S)-2-hydroxy-1-methylethyl]glycyl}-5-methyl-1,2,3,4-tetrahydro-6-isoquinolinyl)-1,2,4-oxadiazol-5-yl]-2-[(1-methylethyl)oxy]benzonitrile (1:1) as an off-white solid (20 mg).

LCMS (Method formate): Retention time 0.97 min, MH⁺=490

### Example 131

formic acid-5-(3-{2-[N-(2-hydroxyethyl)glycyl]-5-methyl-1,2,3,4-tetrahydro-6-isoquinolinyl}-1,2,4-oxadiazol-5-yl)-2-[(1-methylethyl)oxy]benzonitrile (1:1)

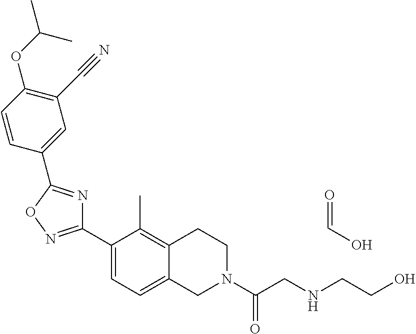

2-Aminoethanol (61 μl, 1 mmol) was added to a stirred mixture of 5-{3-[2-(bromoacetyl)-5-methyl-1,2,3,4-tetrahydro-6-isoquinolinyl]-1,2,4-oxadiazol-5-yl}-2-[(1-methylethyl)oxy]benzonitrile (Preparation 131) (100 mg, 0.2 mmol) and potassium carbonate (70 mg, 0.5 mmol) in acetonitrile (3 ml). The reaction mixture was stirred at room temperature for 3 h. The reaction mixture was filtered and the solvent evaporated from the filtrate. The residue was chromatographed (methanol/DCM, 5-10%). The residue was further purified by MDAP (Method formate). The solvent was evaporated and the residue triturated with diethyl ether to give formic acid-5-(3-{2-[N-(2-hydroxyethyl)glycyl]-5-methyl-1,2,3,4-tetrahydro-6-isoquinolinyl}-1,2,4-oxadiazol-5-yl)-2-[(1-methylethyl)oxy]benzonitrile (1:1) as an off-white solid (25 mg).

LCMS (Method formate): Retention time 0.94 min, MH⁺=476

### Example 132

### 5-(3-{2-[(2S)-2,3-dihydroxypropyl]-5-methyl-1,2,3,4-tetrahydro-6-isoquinolinyl}-1,2,4-oxadiazol-5-yl)-2-[(1-methylethyl)oxy]-3-pyridinecarbonitrile

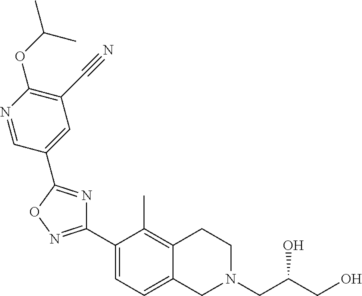

D-Glyceraldehyde (138 mg, 1.5 mmol) was added to a stirred solution of 2-[(1-methylethyl)oxy]-5-[3-(5-methyl-1,2,3,4-tetrahydro-6-isoquinolinyl)-1,2,4-oxadiazol-5-yl]-3-pyridinecarbonitrile trifluoroacetate (Preparation 3) (150 mg, 0.31 mmol) in 1,2-dichloroethane (2 ml), THF (2 ml) and methanol (0.5 ml). The reaction mixture was stirred at room temperature for 30 min then treated with sodium triacetoxyborohydride (325 mg, 1.5 mmol). The reaction mixture was stirred at room temperature for 6 h. Saturated sodium hydrogen carbonate (10 ml) was added and the mixture extracted with ethyl acetate (3×10 ml). The combined organic extracts were washed with water and brine, dried and evaporated. The residue was chromatographed (methanol/DCM, 0-10%) to give 5-(3-{2-[(2S)-2,3-dihydroxypropyl]-5-methyl-1,2,3,4-tetrahydro-6-isoquinolinyl}-1,2,4-oxadiazol-5-yl)-2-[(1-methylethyl)oxy]-3-pyridinecarbonitrile as a colourless solid (56 mg).

LCMS (Method formate): Retention time 0.90 min, MH⁺=450.

### Example 133

### 5-(3-{24(2R)-2,3-dihydroxypropyl]-5-methyl-1,2,3,4-tetrahydro-6-isoquinolinyl}-1,2,4-oxadiazol-5-yl)-2-[(1-methylethyl)oxy]-3-pyridinecarbonitrile

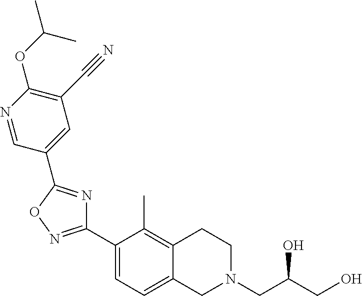

L-Glyceraldehyde (138 mg, 1.5 mmol) was added to a stirred solution of 2-[(1-methylethyl)oxy]-5-[3-(5-methyl-1,2,3,4-tetrahydro-6-isoquinolinyl)-1,2,4-oxadiazol-5-yl]-3-pyridinecarbonitrile trifluoroacetate (Preparation 3) (150 mg, 0.31 mmol) in 1,2-dichloroethane (2 ml), THF (2 ml) and methanol (0.5 ml). The reaction mixture was stirred at room temperature for 30 min then treated with sodium triacetoxyborohydride (325 mg, 1.5 mmol). The reaction mixture was stirred at room temperature for 6 h. Saturated sodium hydrogen carbonate (10 ml) was added and the mixture extracted with ethyl acetate (3×10 ml). The combined organic extracts were washed with water and brine, dried and evaporated. The residue was chromatographed (methanol/DCM, 0-10%) to give 5-(3-{2-[(2R)-2,3-dihydroxypropyl]-5-methyl-1,2,3,4-tetrahydro-6-isoquinolinyl}-1,2,4-oxadiazol-5-yl)-2-[(1-methylethyl)oxy]-3-pyridinecarbonitrile (80 mg) as a colourless solid.

LCMS (Method formate): Retention time 0.90 min, MH⁺=450

### Example 134

### 5-(3-{2-[2-hydroxy-1-(hydroxymethyl)ethyl]-5-methyl-1,2,3,4-tetrahydro-6-isoquinolinyl}-1,2,4-oxadiazol-5-yl)-2-[(1-methylethyl)oxy]-3-pyridinecarbonitrile hydrochloride

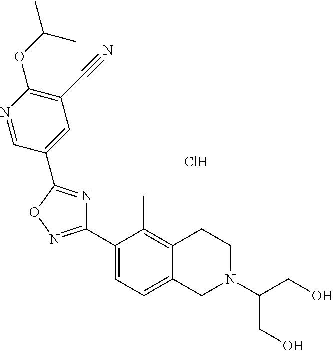

To 5-{3-[2-(2,2-dimethyl-1,3-dioxan-5-yl)-5-methyl-1,2,3,4-tetrahydro-6-isoquinolinyl]-1,2,4-oxadiazol-5-yl}-2-[(1-methylethyl)oxy]-3-pyridinecarbonitrile (Preparation 5) (363 mg, 0.74 mmol) in THF (6 ml) was added aqueous hydrochloric acid (2M, 6.0 ml, 200 mmol) and the reaction mixture stirred at room temperature for 2 h. The reaction was reduced to dryness under a stream of nitrogen. Ethyl acetate (25 ml) was added to the residue and the mixture was treated with saturated sodium hydrogen carbonate solution (30 ml). These two layers were filtered through a frit to remove the residual insoluble material; the organic was separated and concentrated under reduced pressure to give a beige solid. The solid was dissolved in 1,4-dioxane (5 ml), treated with hydrogen chloride in diethyl ether (1 M, 0.6 ml) and stirred for 5 min. The solvent was evaporated under vacuum to give 5-(3-{2-[2-hydroxy-1-(hydroxymethyl)ethyl]-5-methyl-1,2,3,4-tetrahydro-6-isoquinolinyl}-1,2,4-oxadiazol-5-yl)-2-[(1-methylethyl)oxy]-3-pyridinecarbonitrile hydrochloride (132 mg) as a beige powder.

LCMS (Method formate): Retention time 0.88 min, MH⁺=450

1H NMR (D6-DMSO): δ H 10.24 (1H, bs), 9.22 (1H, d), 9.00 (1H, d), 7.79 (1H, d), 7.30 (1H, d), 5.54-5.48 (3H, m), 4.73 (1H, m), 4.61 (1H, m), 3.89 (5H, m), 3.55 (1H, m), 3.41 (1H, m—partially obscured by water), 3.14 (2H, m), 2.48 (3H, s), 1.42 (6H, d).

The residue from the filtration was dried, dissolved in methanol/DCM and applied to a silica cartridge (50 g). The cartridge was dried in vacuo and then eluted with a methanol/DCM gradient (0-15%). The product fractions were combined and concentrated in vacuo. The residual solid was dissolved in 1,4-dioxane (3 ml), treated with hydrogen chloride in diethyl ether (1 M, 0.4 ml) and stirred for 5 min. The mixture was concentrated under reduced pressure give 5-(3-{2-[2-hydroxy-1-(hydroxymethyl)ethyl]-5-methyl-1,2,3,4-tetrahydro-6-isoquinolinyl}-1,2,4-oxadiazol-5-yl)-2-[(1-methylethyl)oxy]-3-pyridinecarbonitrile hydrochloride as a beige powder (41 mg).

LCMS (Method formate): Retention time 0.89 min, MH⁺=450

1H NMR (D6-DMSO): δ H 10.22 (1H, bs), 9.22 (1H, d), 9.00 (1H, d), 7.79 (1H, d), 7.29 (1H, d), 5.54-5.48 (3H, m), 4.73 (1H, m), 4.61 (1H, m), 3.89 (5H, m), 3.55 (1H, m), 3.41 (1H, m), 3.14 (2H, m), 2.47 (3H, s), 1.41 (6H, d).

The two batches of 5-(3-{2-[2-hydroxy-1-(hydroxymethyl)ethyl]-5-methyl-1,2,3,4-tetrahydro-6-isoquinolinyl}-1,2,4-oxadiazol-5-yl)-2-[(1-methylethyl)oxy]-3-pyridinecarbonitrile hydrochloride were combined (173 mg).

### Example 135

### 5-(3-{3-[(2S)-2,3-dihydroxypropyl]-2,3,4,5-tetrahydro-1H-3-benzazepin-7-yl}-1,2,4-oxadiazol-5-yl)-2-[(2,2,2-trifluoroethyl)oxy]benzonitrile

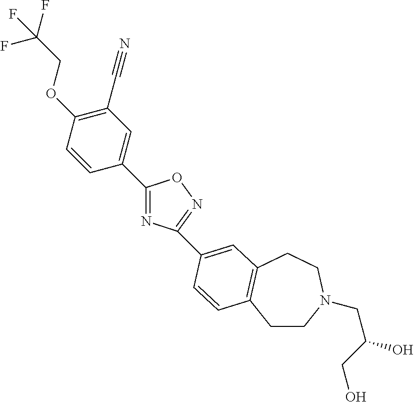

D-Glyceraldehyde (150 mg, 1.7 mmol) was added to a stirred solution of 5-[3-(2,3,4,5-tetrahydro-1H-3-benzazepin-7-yl)-1,2,4-oxadiazol-5-yl]-2-[(2,2,2-trifluoroethyl)oxy]benzonitrile hydrochloride (Preparation 6) (150 mg, 0.33 mmol) in 1,2-dichloroethane (2 ml), THF (2 ml) and methanol (1 ml). The reaction mixture was stirred at room temperature for 30 min then treated with sodium triacetoxyborohydride (353 mg, 1.7 mmol). The reaction mixture was stirred at room temperature for 6 h. Saturated sodium hydrogen carbonate (10 ml) was added and the mixture extracted with ethyl acetate (3×10 ml). The combined organic extracts were washed with water and brine, dried and evaporated. The residue was chromatographed (methanol/DCM, 0-10% methanol) and the residue triturated with diethyl ether to give 5-(3-{3-[(2S)-2,3-dihydroxypropyl]-2,3,4,5-tetrahydro-1H-3-benzazepin-7-yl}-1,2,4-oxadiazol-5-yl)-2-[(2,2,2-trifluoroethyl)oxy]benzonitrile as a colourless solid (77 mg).

LCMS (Method formate): Retention time 0.88 min, MH⁺=489

### Example 136

### 2-[6-(5-{3-chloro-4-[(1-methylethyl)oxy]phenyl}-1,2,4-oxadiazol-3-yl)-5-methyl-3,4-dihydro-2(1H)-isoquinolinyl]-N-(2-hydroxyethyl)acetamide hydrochloride

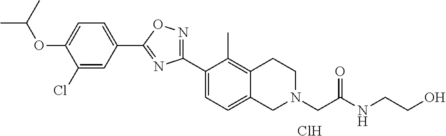

Finely crushed [6-(5-{3-chloro-4-[(1-methylethyl)oxy]phenyl}-1,2,4-oxadiazol-3-yl)-5-methyl-3,4-dihydro-2(1H)-isoquinolinyl]acetic acid (Preparation 10) (88 mg, 0.2 mmol) was suspended in a mixture of DMF (2 ml) and NMP (2.0 ml). The solution was warmed and then cooled to room temperature. DIPEA (0.11 ml, 0.6 mmol) was added, then HATU (76 mg, 0.2 mmol) and after 5 min ethanolamine (0.018 ml, 0.30 mmol) and stirring continued for 15 min. The reaction was partitioned between ethyl acetate and saturated sodium hydrogen carbonate, the aqueous extracted and the combined organic phases washed with saturated sodium hydrogen carbonate (x3), dried (MgSO₄) and concentrated in vacuo. Purification of the residual solid by MDAP (Method HpH) was attempted, during which process some product crystallised. This was triturated with diethyl ether and isolated by filtration. The solid was dissolved in 1,4-dioxane (3 ml) and treated with hydrogen chloride in 1,4-dioxane (4N, 1 ml). The mixture was concentrated in vacuo and the residue dried under vacuum for 12 h to give 2-[6-(5-{3-chloro-4-[(1-methylethyl)oxy]phenyl}-1,2,4-oxadiazol-3-yl)-5-methyl-3,4-dihydro-2(1H)-isoquinolinyl]-N-(2-hydroxyethyl)acetamide hydrochloride (17 mg, 16%) as a white solid.

LCMS (Method HpH): Retention time 1.31 min, MH⁺=485 The purified material from the MDAP was suspended in DCM, dried (hydrophobic frit) and concentrated in vacuo. The residue was dissolved in 1,4-dioxane (3 ml) and treated with hydrogen chloride in 1,4-dioxane (4N, 1 ml). The mixture was concentrated in vacuo and the residue dried under vacuum for 12 h to give 2-[6-(5-{3-chloro-4-[(1-methylethyl)oxy]phenyl}-1,2,4-oxadiazol-3-yl)-5-methyl-3,4-dihydro-2(1H)-isoquinolinyl]-N-(2-hydroxyethyl)acetamide hydrochloride (38 mg, 36%) as a white solid.

LCMS (Method HpH): Retention time 1.31 min, MH⁺=485

### Example 137

### 5-[3-(2-glycyl-5-methyl-1,2,3,4-tetrahydro-6-isoquinolinyl)-1,2,4-oxadiazol-5-yl]-2-[(1-methylethyl)oxy]-3-pyridinecarbonitrile hydrochloride

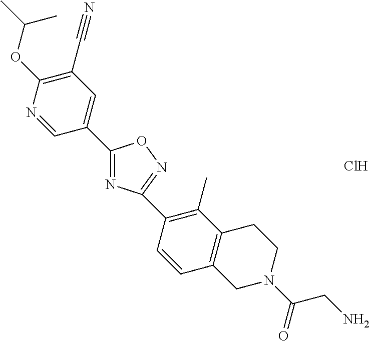

Hydrogen chloride in 1,4-dioxane (1 ml) was added to a stirred solution of 1,1-dimethylethyl {2-[6-(5-{5-cyano-6-[(1-methylethyl)oxy]-3-pyridinyl}-1,2,4-oxadiazol-3-yl)-5-methyl-3,4-dihydro-2(1H)-isoquinolinyl]-2-oxoethyl}carbamate (Preparation 13) (170 mg, 0.32 mmol) in 1,4-dioxane (1 ml). The reaction mixture was stirred at room temperature for 3 h. Ether (10 ml) was added and the mixture stirred for 20 min. The solid was isolated by filtration, washed with diethyl ether and dried to give 5-[3-(2-glycyl-5-methyl-1,2,3,4-tetrahydro-6-isoquinolinyl)-1,2,4-oxadiazol-5-yl]-2-[(1-methylethyl)oxy]-3-pyridinecarbonitrile hydrochloride as a colourless solid (100 mg).

LCMS (Method formate): Retention time 0.95 min, MH⁺=433

### Example 138

### 5-(3-{3-[(2S)-2,3-dihydroxypropyl]-8-methyl-2,3,4,5-tetrahydro-1H-3-benzazepin-7-yl}-1,2,4-oxadiazol-5-yl)-2-[(1-methylethyl)oxy]benzonitrile

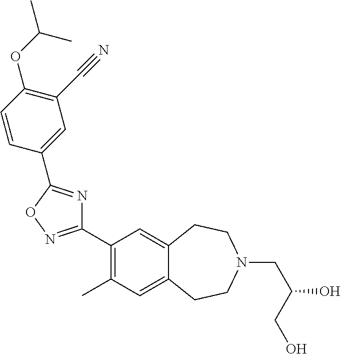

D-Glyceraldehyde (134 mg, 1.5 mmol) was added to a stirred solution of 2-[(1-methylethyl)oxy]-5-[3-(8-methyl-2,3,4,5-tetrahydro-1H-3-benzazepin-7-yl)-1,2,4-oxadiazol-5-yl]benzonitrile trifluoroacetate (Preparation 14) (150 mg, 0.30 mmol) in 1,2-dichloroethane (2 ml), THF (2 ml) and methanol (1 ml). The reaction mixture was stirred at room temperature for 30 min then treated with sodium triacetoxyborohydride (316 mg, 1.5 mmol). The reaction mixture was stirred at room temperature overnight. Saturated sodium hydrogen carbonate (10 ml) was added and the mixture extracted with ethyl acetate (3×10 ml). The combined extracts were washed with water and brine, dried and evaporated. The residue was chromatographed (methanol/DCM, 0-5%). Trituration with diethyl ether gave 5-(3-{3-[(2S)-2,3-dihydroxypropyl]-8-methyl-2,3,4,5-tetrahydro-1H-3-benzazepin-7-yl}-1,2,4-oxadiazol-5-yl)-2-[(1-methylethyl)oxy]benzonitrile as a colourless solid (55 mg).

LCMS (Method formate): Retention time 0.89 min, MH⁺=463

### Example 139

### 2-({2-[6-(5-{3-chloro-4-[(1-methylethyl)oxy]phenyl}-1,2,4-oxadiazol-3-yl)-5-methyl-3,4-dihydro-2(1H)-isoquinolinyl]-2-oxoethyl}amino)ethanol hydrochloride

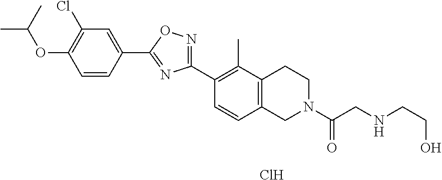

Acetonitrile (3 ml) was added to 2-(bromoacetyl)-6-(5-{3-chloro-4-[(1-methylethyl)oxy]phenyl}-1,2,4-oxadiazol-3-yl)-5-methyl-1,2,3,4-tetrahydroisoquinoline (Preparation 132) (101 mg, 0.2 mmol) and potassium carbonate (69 mg, 0.50 mmol) at room temperature and the resulting mixture was treated with ethanolamine (0.060 ml, 1.0 mmol) then stirred at for 2 h. Potassium carbonate (35 mg) and ethanolamine (0.030 ml) were added and the mixture stirred for 30 min. Most of the solvent was removed and the residue partitioned between ethyl acetate and saturated sodium hydrogen carbonate. The aqueous was extracted and the combined organic phases dried (MgSO₄) and concentrated in vacuo to give a residue which was purified by MDAP (Method HpH). The residue was dissolved in DCM, dried (hydrophobic frit) and the solvent was removed in vacuo. The residue was dissolved in 1,4-dioxane (3 ml) and treated with hydrogen chloride in 1,4-dioxane (4N, 1 ml). The mixture was concentrated in vacuo, the residue triturated with diethyl ether and the solid isolated by filtration to give 2-({2-[6-(5-{3-chloro-4-[(1-methylethyl)oxy]phenyl}-1,2,4-oxadiazol-3-yl)-5-methyl-3,4-dihydro-2(1H)-isoquinolinyl]-2-oxoethyl}amino)ethanol hydrochloride (15 mg, 14%) as a white solid.

LCMS (Method HpH): Retention time 1.26 min, MH⁺=485

### Example 140

### 5-[3-(3-glycyl-6-methyl-2,3,4,5-tetrahydro-1H-3-benzazepin-7-yl)-1,2,4-oxadiazol-5-yl]-2-[(1-methylethyl)oxy]benzonitrile hydrochloride

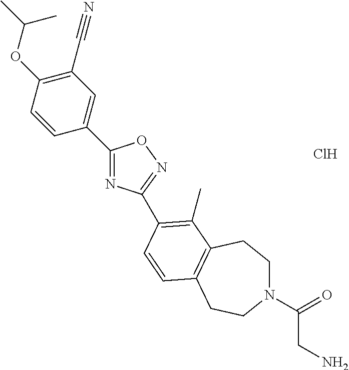

Hydrogen chloride in 1,4dioxane (4M, 1 ml) was added to a solution of 1,1-dimethylethyl {2-[7-(5-{3-cyano-4-[(1-methylethyl)oxy]phenyl}-1,2,4-oxadiazol-3-yl)-6-methyl-1,2,4,5-tetrahydro-3H-3-benzazepin-3-yl]-2-oxoethyl}carbamate (Preparation 133) (100 mg, 0.18 mmol) in 1,4-dioxane (1 ml). The reaction mixture was allowed to stand at room temperature overnight. The solvent was evaporated and the residue triturated with diethyl ether to give 5-[3-(3-glycyl-6-methyl-2,3,4,5-tetrahydro-1H-3-benzazepin-7-yl)-1,2,4-oxadiazol-5-yl]-2-[(1-methylethyl)oxy]benzonitrile hydrochloride as a colourless solid (76 mg).

LCMS (Method formate): Retention time 0.95 min, MH⁺=446

### Example 141 {2-[6-(5-{5-chloro-6-[(1-methylethyl)oxy]-3-pyridinyl}-1,2,4-oxadiazol-3-yl)-5-methyl-3,4-dihydro-2(1H)-isoquinolinyl]-2-oxoethyl}amine

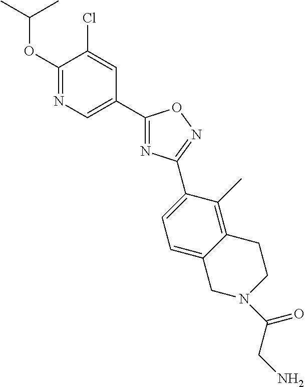

To 1,1-dimethylethyl {2-[6-(5-{5-chloro-6-[(1-methylethyl)oxy]-3-pyridinyl}-1,2,4-oxadiazol-3-yl)-5-methyl-3,4-dihydro-2(1H)-isoquinolinyl]-2-oxoethyl}carbamate (Preparation 139) (62 mg, 0.11 mmol) in DCM (2 ml) was added trifluoroacetic acid (200 μl, 2.6 mmol) and the reaction mixture stirred at room temperature for 45 min. The reaction mixture was applied an aminopropyl SPE (2 g) and the SPE eluted using methanol in DCM (10%). The appropriate fractions were combined and dried under a stream of nitrogen. The residue was dissolved in DMSO (1 ml) and purified by MDAP (Method HpH). The solvent was evaporated under a stream of nitrogen to give the required product {2-[6-(5-{5-chloro-6-[(1-methylethyl)oxy]-3-pyridinyl}-1,2,4-oxadiazol-3-yl)-5-methyl-3,4-dihydro-2(1H)-isoquinolinyl]-2-oxoethyl}amine (38 mg).

LCMS (Method formate): Retention time 1.07 min, MH⁺=442/444

### Example 142

### 5-(3-{34[(2S)-2,3-dihydroxypropyl]-2,3,4,5-tetrahydro-1H-3-benzazepin-7-yl}-1,2,4-oxadiazol-5-yl)-2-(propylamino)-3-pyridinecarbonitrile

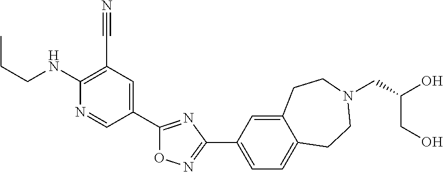

To D-(+)-glyceraldehyde (195 mg, 2.2 mmol) in DCM) (3 ml) under nitrogen, was added acetic acid (0.031 ml, 0.54 mmol) followed by 2-(propylamino)-5-[3-(2,3,4,5-tetrahydro-1H-3-benzazepin-7-yl)-1,2,4-oxadiazol-5-yl]-3-pyridinecarbonitrile (Preparation 142) (135 mg, 0.36 mmol) and sodium triacetoxyborohydride (497 mg, 2.3 mmol). The mixture was stirred at room temperature for 65 h, saturated aqueous sodium hydrogen carbonate solution (5 ml) was added and the mixture stirred vigorously for 15 min. The phases were separated and the aqueous phase was extracted with DCM (3×4 ml). The aqueous phase was further extracted with ethyl acetate (3×4 ml). The aqueous phase was basified to pH12 with aqueous sodium hydroxide solution (1M, ca 2 ml) and then further extracted with DCM (3×4 ml). The combined organic phases were dried (hydrophobic frit), and the solvent was evaporated under a stream of nitrogen to give a residue which was purified by MDAP (Method formate). The required fractions were evaporated in vacuo to give 5-(3-{3-[(2S)-2,3-dihydroxypropyl]-2,3,4,5-tetrahydro-1H-3-benzazepin-7-yl}-1,2,4-oxadiazol-5-yl)-2-(propylamino)-3-pyridinecarbonitrile as a pale brown gum, (61 mg, 38%).

LCMS (Method formate): Retention time 0.87 min, MH⁺=449

### Example 143

### 5-(3-{34[(2S)-2,3-dihydroxypropyl]-2,3,4,5-tetrahydro-1H-3-benzazepin-7-yl}-1,2,4-oxadiazol-5-yl)-2-(propylamino)-3-pyridinecarbonitrile hydrochloride

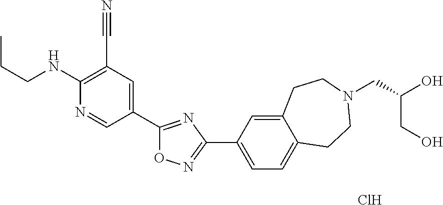

To a stirred solution of 5-(3-{3-[(2S)-2,3-dihydroxypropyl]-2,3,4,5-tetrahydro-1H-3-benzazepin-7-yl}-1,2,4-oxadiazol-5-yl)-2-(propylamino)-3-pyridinecarbonitrile (Example 142) (61 mg, 0.14 mmol) in methanol (3 ml) was added hydrogen chloride in diethyl ether (1M, 0.68 ml, 0.68 mmol). The mixture was allowed to stand for 10 min at room temperature before being evaporated under a stream of nitrogen, triturated with diethyl ether and then dried in vacuo to give 5-(3-{3-[(2S)-2,3-dihydroxypropyl]-2,3,4,5-tetrahydro-1H-3-benzazepin-7-yl}-1,2,4-oxadiazol-5-yl)-2-(propylamino)-3-pyridinecarbonitrile hydrochloride as a cream solid (63 mg, 95%).

LCMS (Method formate): Retention time 0.86 min, MH⁺=449

### Example 144

### 2-[6-(5-{5-chloro-6-[(1-methylethyl)oxy]-3-pyridinyl}-1,2,4-oxadiazol-3-yl)-5-methyl-3,4-dihydro-2(1H)-isoquinolinyl]-1,3-propanediol

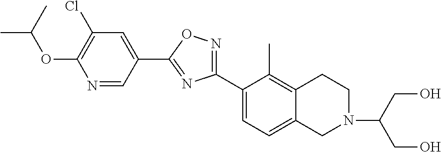

To 6-(5-{5-chloro-6-[(1-methylethyl)oxy]-3-pyridinyl}-1,2,4-oxadiazol-3-yl)-2-(2,2-dimethyl-1,3-dioxan-5-yl)-5-methyl-1,2,3,4-tetrahydroisoquinoline (Preparation 147) (190 mg, 0.38 mmol) in THF (2 ml) was added aqueous hydrochloric acid (2M, 2 ml, 66 mmol) and the reaction mixture stirred at room temperature for 6 h. The solvent was evaporated under a stream of nitrogen and the residue partitioned between DCM/ethyl acetate (1:1, 3×10 ml) and saturated aqueous sodium hydrogen carbonate solution (10 ml). The organics were combined, dried (hydrophobic frit) and evaporated under a stream of nitrogen. The sample was dissolved in DMSO (1 ml) and purified by MDAP (Method HpH). The solvent was evaporated under a stream of nitrogen to give 2-[6-(5-{5-chloro-6-[(1-methylethyl)oxy]-3-pyridinyl}-1,2,4-oxadiazol-3-yl)-5-methyl-3,4-dihydro-2(1H)-isoquinolinyl]-1,3-propanediol (94 mg, 54%).

LCMS (Method formate): Retention time 1.02 min, MH⁺=459/461

### Example 145

### 5-(3-{34[(2S)-2,3-dihydroxypropyl]-6-methyl-2,3,4,5-tetrahydro-1H-3-benzazepin-7-yl}-1,2,4-oxadiazol-5-yl)-2-(propylamino)-3-pyridinecarbonitrile

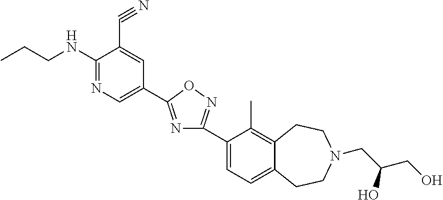

To 5-[3-(6-methyl-2,3,4,5-tetrahydro-1H-3-benzazepin-7-yl)-1,2,4-oxadiazol-5-yl]-2-(propylamino)-3-pyridinecarbonitrile trifluoroacetate (Preparation 148) (98 mg, 0.25 mmol) in methanol (1.0 ml) and DCM (3 m) was added D-glyceraldehyde (114 mg, 1.3 mmol) and acetic acid (0.015 ml, 0.27 mmol). The mixture was stirred at room temperature for 30 min. Sodium triacetoxyborohydride (267 mg, 1.3 mmol) was added and the mixture was stirred for 1 h. Water (10 ml) was added and the mixture extracted with DCM (3×10 ml). The organics were combined, dried (hydrophobic frit) and evaporated in vacuo. The sample was dissolved in DMSO (1 ml) and purified by MDAP (Method HpH). The solvent was evaporated in vacuo to give 5-(3-{3-[(2S)-2,3-dihydroxypropyl]-6-methyl-2,3,4,5-tetrahydro-1H-3-benzazepin-7-yl}-1,2,4-oxadiazol-5-yl)-2-(propylamino)-3-pyridinecarbonitrile as a white solid (36 mg).

LCMS (Method formate): Retention time 0.88 min, MH⁺=463

### Example 146

### 2-[7-(5-{3-cyano-4-[(1-methylethyl)oxy]phenyl}-1,2,4-oxadiazol-3-yl)-6-methyl-1,2,4,5-tetrahydro-3H-3-benzazepin-3-yl]-N-(2-hydroxyethyl)acetamide

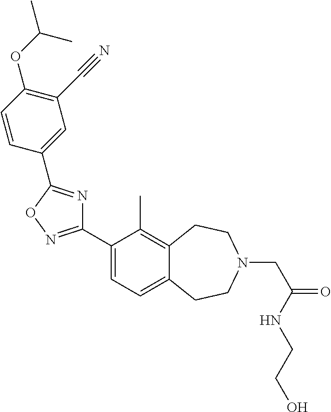

A mixture of [7-(5-{3-cyano-4-[(1-methylethyl)oxy]phenyl}-1,2,4-oxadiazol-3-yl)-6-methyl-1,2,4,5-tetrahydro-3H-3-benzazepin-3-yl]acetic acid trifluoroacetate (Preparation 150) (130 mg), N-ethylmorpholine (74 μl, 0.58 mmol), hydroxybenzotriazole hydrate (53 mg, 0.35 mmol), EDC (67 mg, 0.35 mmol) and ethanolamine (27 mg, 0.44 mmol) in DMF (3 ml) was stirred at room temperature overnight. The reaction mixture was diluted with saturated sodium hydrogen carbonate (10 ml) and extracted with ethyl acetate (2×10 ml). The combined extracts were washed with hydrochloric acid (1M), water and brine. The organic phase was dried and evaporated. Chromatography (methanol/DCM, 0-8%) gave 2-[7-(5-{3-cyano-4-[(1-methylethyl)oxy]phenyl}-1,2,4-oxadiazol-3-yl)-6-methyl-1,2,4,5-tetrahydro-3H-3-benzazepin-3-yl]-N-(2-hydroxyethyl)acetamide (20 mg) as a colourless oil which solidified on trituration with diethyl ether.

LCMS (Method formate): Retention time 0.87 min, MH⁺=490

### Example 147

### 2-[7-(5-{3-cyano-4-[(1-methylethyl)oxy]phenyl}-1,2,4-oxadiazol-3-yl)-6-methyl-1,2,4,5-tetrahydro-3H-3-benzazepin-3-yl]-N-[(2R)-2-hydroxypropyl]acetamide

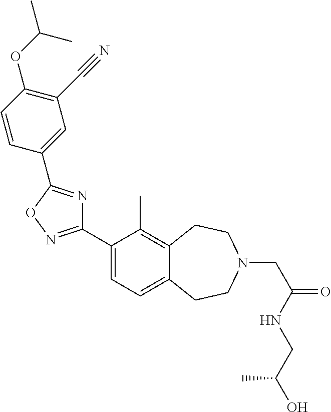

A mixture of [7-(5-{3-cyano-4-[(1-methylethyl)oxy]phenyl}-1,2,4-oxadiazol-3-yl)-6-methyl-1,2,4,5-tetrahydro-3H-3-benzazepin-3-yl]acetic acid trifluoroacetate (Preparation 150) (130 mg), N-ethylmorpholine (74 μl, 0.58 mmol), hydroxybenzotriazole hydrate (53 mg, 0.35 mmol), EDC (67 mg, 0.35 mmol) and (R)-1-amino-2-propanol (33 mg, 0.4 4 mmol) in DMF (3 ml) was stirred at room temperature overnight. The reaction mixture was diluted with saturated sodium hydrogen carbonate (10 ml) and extracted with ethyl acetate (2×10 ml). The combined extracts were washed with hydrochloric acid (1M), water and brine. The organic phase was dried and evaporated. Chromatography (methanol/DCM, 0-8%) gave 2-[7-(5-{3-cyano-4-[(1-methylethyl)oxy]phenyl}-1,2,4-oxadiazol-3-yl)-6-methyl-1,2,4,5-tetrahydro-3H-3-benzazepin-3-yl]-N-[(2R)-2-hydroxypropyl]acetamide (40 mg) as a colourless oil which solidified on trituration with diethyl ether.

LCMS (Method formate): Retention time 0.88 min, MH⁺=504

### Example 148

### 2-[7-(5-{3-cyano-4-[(1-methylethyl)oxy]phenyl}-1,2,4-oxadiazol-3-yl)-6-methyl-1,2,4,5-tetrahydro-3H-3-benzazepin-3-yl]-N-[(2S)-2-hydroxypropyl]acetamide

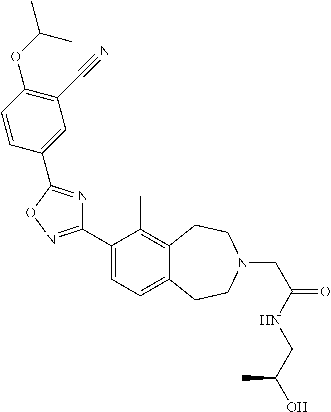

A mixture of [7-(5-{3-cyano-4-[(1-methylethyl)oxy]phenyl}-1,2,4-oxadiazol-3-yl)-6-methyl-1,2,4,5-tetrahydro-3H-3-benzazepin-3-yl]acetic acid trifluoroacetate (Preparation 150) (130 mg), N-ethylmorpholine (74 μl, 0.58 mmol), hydroxybenzotriazole hydrate (53 mg, 0.35 mmol), EDC (67 mg, 0.35 mmol) and (S)-1-amino-2-propanol (33 mg, 0.44 mmol) in DMF (3 ml) was stirred at room temperature overnight. The reaction mixture was diluted with saturated sodium hydrogen carbonate (10 ml) and extracted with ethyl acetate (2×10 ml). The combined extracts were washed with hydrochloric acid (1M), water and brine. The organic phase was dried and evaporated. Chromatography (methanol/DCM, 0-8%) gave 2-[7-(5-{3-cyano-4-[(1-methylethyl)oxy]phenyl}-1,2,4-oxadiazol-3-yl)-6-methyl-1,2,4,5-tetrahydro-3H-3-benzazepin-3-yl]-N-[(2S)-2-hydroxypropyl]acetamide (22 mg) as a colourless oil which solidified on trituration with diethyl ether.

LCMS (Method formate): Retention time 0.88 min, MH⁺=504

### Example 149

### 5-(3-{3-[2-hydroxy-1-(hydroxymethyl)ethyl]-6-methyl-2,3,4,5-tetrahydro-1H-3-benzazepin-7-yl}-1,2,4-oxadiazol-5-yl)-2-(propylamino)-3-pyridinecarbonitrile

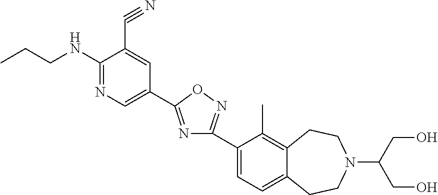

To 5-{3-[3-(2,2-dimethyl-1,3-dioxan-5-yl)-6-methyl-2,3,4,5-tetrahydro-1H-3-benzazepin-7-yl]-1,2,4-oxadiazol-5-yl}-2-(propylamino)-3-pyridinecarbonitrile (Preparation 152) (110 mg, 0.22 mmol) in THF (2 ml) was added aqueous hydrochloric acid (2M, 2 ml, 4.0 mmol) and the reaction was stirred at room temperature for 3 h. The solvent was evaporated in vacuo and the residue partitioned between DCM/ethyl acetate (1:1, 3×10 ml) and saturated sodium hydrogen carbonate solution (10 ml). The organic phases were combined, dried (hydrophobic frit) and evaporated in vacuo to give a yellow solid. The solid was dissolved in DMSO (1 ml) and purified by MDAP (Method HpH). The solvent was evaporated under a stream of nitrogen to give 5-(3-{3-[2-hydroxy-1-(hydroxymethyl)ethyl]-6-methyl-2,3,4,5-tetrahydro-1H-3-benzazepin-7-yl}-1,2,4-oxadiazol-5-yl)-2-(propylamino)-3-pyridinecarbonitrile (12 mg) as a cream/yellow solid.

LCMS (Method formate): Retention time 0.89 min, MH⁺=463

### Example 150

### 5-(3-{3-[2-hydroxy-1-(hydroxymethyl)ethyl]-2,3,4,5-tetrahydro-1H-3-benzazepin-7-yl}-1,2,4-oxadiazol-5-yl)-2-(propylamino)-3-pyridinecarbonitrile dihydrochloride

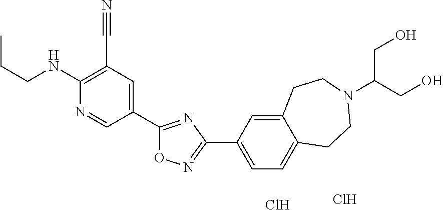

To a stirred solution the impure 5-{3-[3-(2,2-dimethyl-1,3-dioxan-5-yl)-2,3,4,5-tetrahydro-1H-3-benzazepin-7-yl]-1,2,4-oxadiazol-5-yl}-2-(propylamino)-3-pyridinecarbonitrile (Preparation 153) (179 mg, 0.37 mmol) in THF (4 ml) was added hydrochloric acid (2M, 4.0 ml, 12 mmol). The mixture was stirred at room temperature for 85 min. The volatiles were evaporated under a stream of nitrogen and the residue was partitioned between saturated aqueous sodium hydroxide solution (5 ml) and ethyl acetate (5 ml). The phases were separated and the aqueous phase extracted with ethyl acetate (3×4 ml) and DCM (4×4 ml). The combined organic phases were dried (hydrophobic frit) and evaporated to dryness under a stream of nitrogen. The residue was purified by MDAP (Method formate). The required fractions were evaporated in vacuo to give 5-(3-{3-[2-hydroxy-1-(hydroxymethyl)ethyl]-2,3,4,5-tetrahydro-1H-3-benzazepin-7-yl}-1,2,4-oxadiazol-5-yl)-2-(propylamino)-3-pyridinecarbonitrile as a cream solid, (80 mg, 489%)

LCMS (Method formate): Retention time 0.87 min, MH⁺=449

To a stirred solution of 5-(3-{3-[2-hydroxy-1-(hydroxymethyl)ethyl]-2,3,4,5-tetrahydro-1H-3-benzazepin-7-yl}-1,2,4-oxadiazol-5-yl)-2-(propylamino)-3-pyridinecarbonitrile (80 mg, 0.18 mmol) in methanol (6 ml) was added hydrogen chloride in diethyl ether (1M, 0.90 ml, 0.90 mmol). The mixture was allowed to stand for 5 min at room temperature before being evaporated under a stream of nitrogen, the residue wastriturated with diethyl ether and then dried in vacuo to give 5-(3-{3-[2-hydroxy-1-(hydroxymethyl)ethyl]-2,3,4,5-tetrahydro-1H-3-benzazepin-7-yl}-1,2,4-oxadiazol-5-yl)-2-(propylamino)-3-pyridinecarbonitrile dihydrochloride as a cream solid, (86 mg, 92%).

LCMS (Method formate): Retention time 0.86 min, MH⁺=449

### Example 151

### 5-[3-(3-glycyl-2,3,4,5-tetrahydro-1H-3-benzazepin-7-yl)-1,2,4-oxadiazol-5-yl]-2-(propylamino)-3-pyridinecarbonitrile

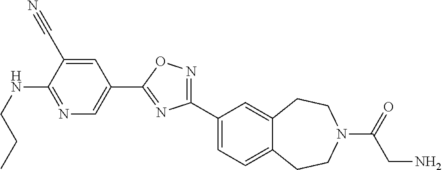

To a stirred solution the impure 1,1-dimethylethyl [2-(7-{5-[5-cyano-6-(propylamino)-3-pyridinyl]-1,2,4-oxadiazol-3-yl}-1,2,4,5-tetrahydro-3H-3-benzazepin-3-yl)-2-oxoethyl]carbamate (Preparation 154) (172 mg, max 0.13 mmol) in THF (2 ml) was added hydrochloric acid (2M, 2.0 ml, 6.0 mmol). The mixture was stirred at room temperature for 2 h. The volatiles were evaporated to dryness under a stream of nitrogen and the residue was purified by MDAP (Method formate). The required fractions were evaporated in vacuo to give 5-[3-(3-glycyl-2,3,4,5-tetrahydro-1H-3-benzazepin-7-yl)-1,2,4-oxadiazol-5-yl]-2-(propylamino)-3-pyridinecarbonitrile as a cream solid (41 mg, 30%).

LCMS (Method formate): Retention time 0.92 min, MH⁺=432

### Example 152

### 5-[3-(3-glycyl-2,3,4,5-tetrahydro-1H-3-benzazepin-7-yl)-1,2,4-oxadiazol-5-yl]-2-(propylamino)-3-pyridinecarbonitrile dihydrochloride

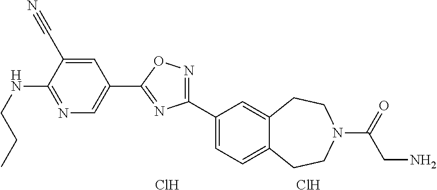

To a stirred solution of 5-[3-(3-glycyl-2,3,4,5-tetrahydro-1H-3-benzazepin-7-yl)-1,2,4-oxadiazol-5-yl]-2-(propylamino)-3-pyridinecarbonitrile (Example 151) (41 mg, 0.096 mmol) in methanol (2 ml) was added hydrogen chloride in diethyl ether (1M, 0.50 ml, 0.50 mmol). The mixture was allowed to stand for 5 min at room temperature before being evaporated under a stream of nitrogen, triturated with diethyl ether and then dried in vacuo to give 5-[5-(3-glycyl-2,3,4,5-tetrahydro-1H-3-benzazepin-7-yl)-1,2,4-oxadiazol-5-yl]-2-(propylamino)-3-pyridinecarbonitrile dihydrochloride as a cream solid (45 mg, 93%).

LCMS (Method formate): Retention time 0.90 min, MH⁺=432

### Example 153

### 5-(3-{3-[(2S)-2,3-dihydroxypropyl]-6-methyl-2,3,4,5-tetrahydro-1H-3-benzazepin-7-yl}-1,2,4-oxadiazol-5-yl)-2-[(1-methylethyl)oxy]benzonitrile hydrochloride

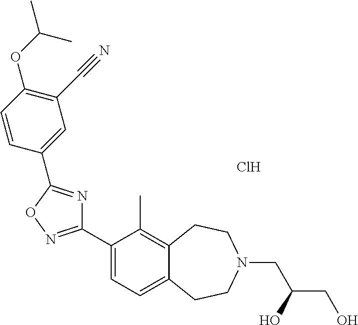

Sodium triacetoxyborohydride (1.05 g, 5.0 mmol) was added portionwise to a stirred solution of 2-[(1-methylethyl)oxy]-5-[3-(6-methyl-2,3,4,5-tetrahydro-1H-3-benzazepin-7-yl)-1,2,4-oxadiazol-5-yl]benzonitrile trifluoroacetate (Preparation 134) (500 mg, 1 mmol) and D-glyceraldehyde (270 mg, 3 mmol) in DCM (10 ml) and methanol (2 ml). The reaction mixture was stirred at room temperature overnight. Saturated sodium hydrogen carbonate (20 ml) was added and the mixture stirred for 15 min. The organic phase was separated. The aqueous phase was extracted with DCM (10 ml). The combined organics were dried and evaporated. The residue was chromatographed (methanol/DCM, 0-4%). The combined product fractions were treated with hydrogen chloride in diethyl ether (1M, 2 ml). The solvent was evaporated and the residue triturated with diethyl ether to give 5-(3-{3-[(2S)-2,3-dihydroxypropyl]-6-methyl-2,3,4,5-tetrahydro-1H-3-benzazepin-7-yl}-1,2,4-oxadiazol-5-yl)-2-[(1-methylethyl)oxy]benzonitrile hydrochloride as a colourless solid (270 mg).

LCMS (Method formate): Retention time 0.85 min, MH⁺=463

### Example 154

### 5-(3-{3-[(2R)-2,3-dihydroxypropyl]-6-methyl-2,3,4,5-tetrahydro-1H-3-benzazepin-7-yl}-1,2,4-oxadiazol-5-yl)-2-[(1-methylethyl)oxy]benzonitrile hydrochloride

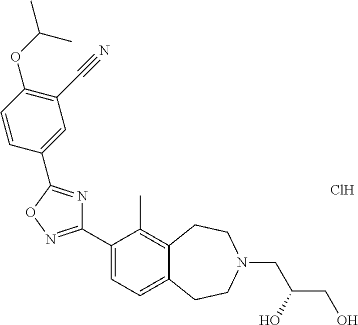

Sodium triacetoxyborohydride (1.05 g, 5.0 mmol) was added portionwise to a stirred solution of 2-[(1-methylethyl)oxy]-5-[3-(6-methyl-2,3,4,5-tetrahydro-1H-3-benzazepin-7-yl)-1,2,4-oxadiazol-5-yl]benzonitrile trifluoroacetate (Preparation 134) (500 mg, 1 mmol) and L-glyceraldehyde (269 mg, 3 mmol) in DCM (10 ml) and methanol (2 ml). The reaction mixture was stirred at room temperature overnight. Saturated sodium hydrogen carbonate (20 ml) was added and the mixture stirred for 15 min. The organic phase was separated. The aqueous phase was extracted with DCM (10 ml). The combined organics were dried and evaporated. The residue was chromatographed (methanol/DCM, 0-4% methanol). The combined product fractions were treated with hydrogen chloride in diethyl ether (1M, 2 ml). The solvent was evaporated and the residue triturated with diethyl ether to give 5-(3-{3-[(2R)-2,3-dihydroxypropyl]-6-methyl-2,3,4,5-tetrahydro-1H-3-benzazepin-7-yl}-1,2,4-oxadiazol-5-yl)-2-[(1-methylethyl)oxy]benzonitrile hydrochloride as a colourless solid (210 mg).

LCMS (Method formate): Retention time 0.85 min, MH⁺=463

### Example 155

### 5-(3-{3-[N-(2-hydroxyethyl)glycyl]-6-methyl-2,3,4,5-tetrahydro-1H-3-benzazepin-7-yl}-1,2,4-oxadiazol-5-yl)-2-[(1-methylethyl)oxy]benzonitrile

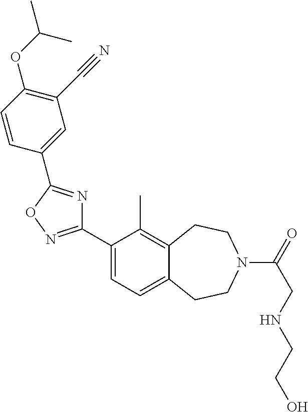

A mixture of 5-{3-[3-(bromoacetyl)-6-methyl-2,3,4,5-tetrahydro-1H-3-benzazepin-7-yl]-1,2,4-oxadiazol-5-yl}-2-[(1-methylethyl)oxy]benzonitrile (Preparation 155) (100 mg, 0.2 mmol), ethanolamine (60 mg, 0.98 mmol), and potassium carbonate (68 mg, 0.49 mmol) in acetonitrile (3 ml) was stirred at room temperature overnight. The solvent was evaporated and the residue chromatographed (methanol/DCM, 0-10%) to give 5-(3-{3-[N-(2-hydroxyethyl)glycyl]-6-methyl-2,3,4,5-tetrahydro-1H-3-benzazepin-7-yl}-1,2,4-oxadiazol-5-yl)-2-[(1-methylethyl)oxy]benzonitrile as a colourless oil which solidified on trituration with diethyl ether (58 mg).

LCMS (Method formate): Retention time 0.99 min, MH⁺=490

### Example 156

### 5-(3-{3-[2-hydroxy-1-(hydroxymethyl)ethyl]-6-methyl-2,3,4,5-tetrahydro-1H-3-benzazepin-7-yl}-1,2,4-oxadiazol-5-yl)-2-[(1-methylethyl)oxy]benzonitrile hydrochloride

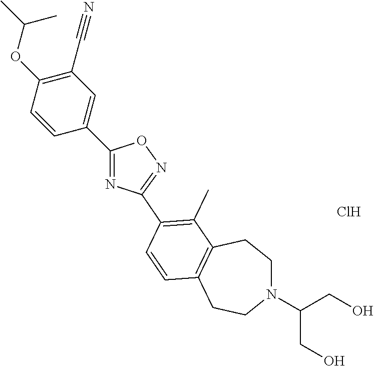

Sodium triacetoxyborohydride (1.05 g, 5.0 mmol) was added portionwise to a stirred solution of 24[(1-methylethyl)oxy]-5-[3-(6-methyl-2,3,4,5-tetrahydro-1H-3-benzazepin-7-yl)-1,2,4-oxadiazol-5-yl]benzonitrile trifluoroacetate (Preparation 134) (500 mg, 1 mmol) and 2,2-dimethyl-1,3-dioxan-5-one (388 mg, 3 mmol) in DCM (10 ml). The reaction mixture was stirred at room temperature overnight. Saturated sodium hydrogen carbonate (20 ml) was added and the mixture stirred for 15 min. The organic phase was separated. The aqueous phase was extracted with DCM (10 ml). The combined organics were dried and evaporated. The residue was dissolved in THF (3 ml) and the solution treated with hydrochloric acid (2M, 3 ml). The reaction mixture was stirred at room temperature for 5 h. Saturated sodium hydrogen carbonate (10 ml) was added and the mixture stirred for 10 min. The organic phase was separated. The aqueous phase was extracted with DCM (10 ml). The combined organics were dried and evaporated. The residue was chromatographed (methanol/DCM, 0-6% methanol). The combined product fractions were treated with hydrogen chloride in diethyl ether (1M, 2 ml). The solvent was evaporated and the residue triturated with diethyl ether to give 5-(3-{3-[2-hydroxy-1-(hydroxymethyl)ethyl]-6-methyl-2,3,4,5-tetrahydro-1H-3-benzazepin-7-yl}-1,2,4-oxadiazol-5-yl)-2-[(1-methylethyl)oxy]benzonitrile hydrochloride as a colourless solid (200 mg).

LCMS (Method formate): Retention time 0.92 min, MH⁺=463

### Example 157

### 2-[7-(5-{3-cyano-4-[(1-methylethyl)oxy]phenyl}-1,2,4-oxadiazol-3-yl)-1,2,4,5-tetrahydro-3H-3-benzazepin-3-yl]-N-(3-hydroxypropyl)acetamide

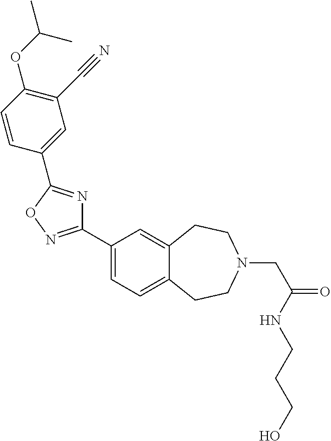

3-Amino-1-propanol (16 mg, 0.21 mmol) was added to a stirred mixture of [7-(5-{3-cyano-4-[(1-methylethyl)oxy]phenyl}-1,2,4-oxadiazol-3-yl)-1,2,4,5-tetrahydro-3H-3-benzazepin-3-yl]acetic acid trifluoroacetate (Preparation 89) (100 mg, 0.18 mmol), DIPEA (64 μl, 0.36 mmol) and HATU (77 mg, 0.2 mmol) in DMF (3 ml). The reaction mixture was stirred at room temperature overnight. The reaction mixture was diluted with ethyl acetate (10 ml). The solution was washed with saturated sodium hydrogen carbonate (10 ml), water (2×20 ml) and brine (10 ml). The organic phase was dried and evaporated. The residue was chromatographed (methanol/DCM, 0-5%). Trituration with diethyl ether gave 2-[7-(5-{3-cyano-4-[(1-methylethyl)oxy]phenyl}-1,2,4-oxadiazol-3-yl)-1,2,4,5-tetrahydro-3H-3-benzazepin-3-yl]-N-(3-hydroxypropyl)acetamide as an off-white solid (41 mg).

LCMS (Method formate): Retention time 0.90 min, MH⁺=490

### Example 158

### 2-[7-(5-{3-cyano-4-[(1-methylethyl)oxy]phenyl}-1,2,4-oxadiazol-3-yl)-6-methyl-1,2,4,5-tetrahydro-3H-3-benzazepin-3-yl]-N-[(1S)-2-hydroxy-1-methylethyl]acetamide

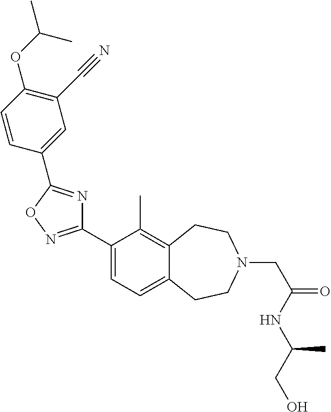

L-Alaninol (15 mg, 0.2 mmol) was added to a stirred mixture of [7-(5-{3-cyano-4-[(1-methylethyl)oxy]phenyl}-1,2,4-oxadiazol-3-yl)-6-methyl-1,2,4,5-tetrahydro-3H-3-benzazepin-3-yl]acetic acid trifluoroacetate (Preparation 150) (80 mg), DIPEA (62 μl, 0.35 mmol) and HATU (75 mg, 0.2 mmol) in DMF (3 ml). The reaction mixture was stirred at room temperature overnight. The reaction mixture was diluted with ethyl acetate (10 ml). The solution was washed with saturated sodium hydrogen carbonate (10 ml), water (2×20 ml) and brine (10 ml). The organic phase was dried and evaporated. The residue was chromatographed (methanol/DCM, 0-5% methanol). Trituration with diethyl ether gave 2-[7-(5-{3-cyano-4-[(1-methylethyl)oxy]phenyl}-1,2,4-oxadiazol-3-yl)-6-methyl-1,2,4,5-tetrahydro-3H-3-benzazepin-3-yl]-N-[(1S)-2-hydroxy-1-methylethyl]acetamide as an off-white solid (51 mg).

LCMS (Method formate): Retention time 0.88 min, MH⁺=504

### Example 159

### 2-[7-(5-{3-cyano-4-[(1-methylethyl)oxy]phenyl}-1,2,4-oxadiazol-3-yl)-6-methyl-1,2,4,5-tetrahydro-3H-3-benzazepin-3-yl]-N-(1R)-2-hydroxy-1-methylethyl]acetamide

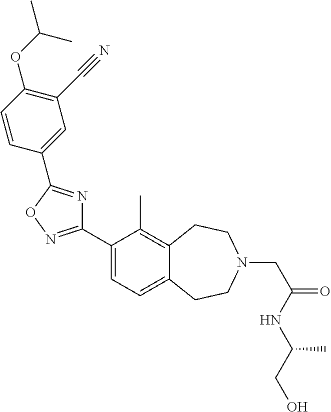

D-Alaninol (15 mg, 0.2 mmol) was added to a stirred mixture of [7-(5-{3-cyano-4-[(1-methylethyl)oxy]phenyl}-1,2,4-oxadiazol-3-yl)-6-methyl-1,2,4,5-tetrahydro-3H-3-benzazepin-3-yl]acetic acid trifluoroacetate (Preparation 150) (80 mg), DIPEA (62 μl, 0.35 mmol) and HATU (75 mg, 0.2 mmol) in DMF (3 ml). The reaction mixture was stirred at room temperature overnight. The reaction mixture was diluted with ethyl acetate (10 ml). The solution was washed with saturated sodium hydrogen carbonate (10 ml), water (2×20 ml) and brine (10 ml). The organic phase was dried and evaporated. The residue was chromatographed (methanol/DCM, 0-5% methanol).

Trituration with diethyl ether gave 2-[7-(5-{3-cyano-4-[(1-methylethyl)oxy]phenyl}-1,2,4-oxadiazol-3-yl)-6-methyl-1,2,4,5-tetrahydro-3H-3-benzazepin-3-yl]-N-(1R)-2-hydroxy-1-methylethyl]acetamide as an off-white solid (42 mg).

LCMS (Method formate): Retention time 0.87 min, MH⁺=504

### Example 160

### 2-[7-(5-{3-cyano-4-[(1-methylethyl)oxy]phenyl}-1,2,4-oxadiazol-3-yl)-1,2,4,5-tetrahydro-3H-3-benzazepin-3-yl]-N-(2-hydroxyethyl)-2-methylpropanamide hydrochloride

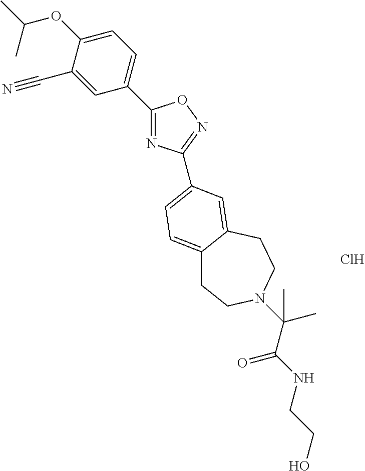

A mixture of 2-[7-(5-{3-cyano-4-[(1-methylethyl)oxy]phenyl}-1,2,4-oxadiazol-3-yl)-1,2,4,5-tetrahydro-3H-3-benzazepin-3-yl]-2-methylpropanoic acid trifluoroacetate (Preparation 156) (60 mg), ethanolamine (9 mg, 0.15 mmol), HATU (55 mg, 0.14 mmol) and DIPEA (46 μl, 0.26 mmol) in DMF (3 ml) was stirred at room temperature overnight. The reaction mixture was diluted with ethyl acetate (10 ml). The mixture was washed with saturated sodium hydrogen carbonate, water and brine. The organic phase was dried and evaporated. The residue was chromatographed (methanol/DCM, 0-5% methanol). The product was dissolved in ethyl acetate (5 ml) and treated with hydrogen chloride in diethyl ether (1M, 0.3 ml). The solvent was evaporated and the residue triturated with diethyl ether to give 2-[7-(5-{3-cyano-4-[(1-methylethyl)oxy]phenyl}-1,2,4-oxadiazol-3-yl)-1,2,4,5-tetrahydro-3H-3-benzazepin-3-yl]-N-(2-hydroxyethyl)-2-methylpropanamide hydrochloride as a colourless solid (33 mg).

LCMS (Method formate): Retention time 0.92 min, MH⁺=504

### Example 161

### 2-[7-(5-{3-cyano-4-[(1-methylethyl)oxy]phenyl}-1,2,4-oxadiazol-3-yl)-1,2,4,5-tetrahydro-3H-3-benzazepin-3-yl]-N-(2-hydroxyethyl)propanamide hydrochloride

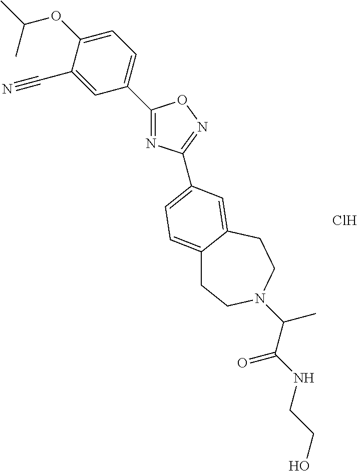

A mixture of 2-[7-(5-{3-cyano-4-[(1-methylethyl)oxy]phenyl}-1,2,4-oxadiazol-3-yl)-1,2,4,5-tetrahydro-3H-3-benzazepin-3-yl]propanoic acid trifluoroacetate (Preparation 158) (100 mg), ethanolamine (15 mg, 0.25 mmol), HATU (94 mg, 0.25 mmol) and DIPEA (58 mg, 0.45 mmol) in DMF (3 ml) was stirred at room temperature overnight. The reaction mixture was diluted with ethyl acetate (10 ml). The mixture was washed with saturated sodium hydrogen carbonate, water and brine. The organic phase was dried and evaporated. The residue was chromatographed (methanol/DCM, 0-5%). The product was dissolved in ethyl acetate (5 ml) and treated with hydrogen chloride in diethyl ether (1M, 0.3 ml). The solvent was evaporated and the residue triturated with diethyl ether to give 2-[7-(5-{3-cyano-4-[(1-methylethyl)oxy]phenyl}-1,2,4-oxadiazol-3-yl)-1,2,4,5-tetrahydro-3H-3-benzazepin-3-yl]-N-(2-hydroxyethyl)propanamide hydrochloride as a colourless solid (96 mg).

LCMS (Method formate): Retention time 0.89 min, MH⁺=490

### Example 162

### 2-[7-(5-{3-cyano-4-[(1-methylethyl)oxy]phenyl}-1,2,4-oxadiazol-3-yl)-1,2,4,5-tetrahydro-3H-3-benzazepin-3-yl]-N-(1R)-2-hydroxy-1-methylethyl]propanamide hydrochloride

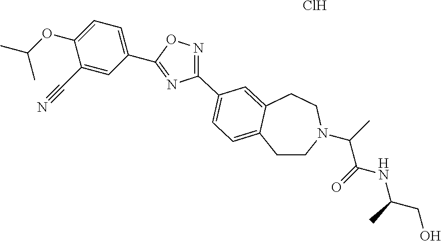

A mixture of 2-[7-(5-{3-cyano-4-[(1-methylethyl)oxy]phenyl}-1,2,4-oxadiazol-3-yl)-1,2,4,5-tetrahydro-3H-3-benzazepin-3-yl]propanoic acid trifluoroacetate (Preparation 158) (100 mg), (2R)-2-amino-1-propanol (19 mg, 0.25 mmol), HATU (94 mg, 0.25 mmol) and DIPEA (58 mg, 0.45 mmol) in DMF (3 ml) was stirred at room temperature overnight. The reaction mixture was diluted with ethyl acetate (10 ml). The mixture was washed with saturated sodium hydrogen carbonate, water and brine. The organic phase was dried and evaporated. The residue was chromatographed (methanol/DCM, 0-5% methanol). The product was dissolved in ethyl acetate (5 ml) and treated with hydrogen chloride in diethyl ether (1M, 0.3 ml). The solvent was evaporated and the residue triturated with diethyl ether to give 2-[7-(5-{3-cyano-4-[(1-methylethyl)oxy]phenyl}-1,2,4-oxadiazol-3-yl)-1,2,4,5-tetrahydro-3H-3-benzazepin-3-yl]-N-(1R)-2-hydroxy-1-methylethyl]propanamide hydrochloride as a colourless solid (106 mg).

LCMS (Method formate): Retention time 0.91 min, MH⁺=504

### Example 163

### 2-[7-(5-{3-cyano-4-[(1-methylethyl)oxy]phenyl}-1,2,4-oxadiazol-3-yl)-1,2,4,5-tetrahydro-3H-3-benzazepin-3-yl]-N-(1R)-2-hydroxy-1-methylethyl]-2-methylpropanamide hydrochloride

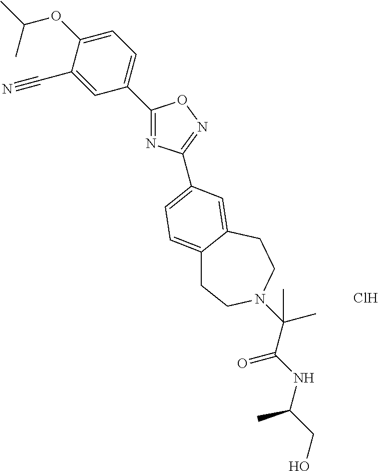

A mixture of 2-[7-(5-{3-cyano-4-[(1-methylethyl)oxy]phenyl}-1,2,4-oxadiazol-3-yl)-1,2,4,5-tetrahydro-3H-3-benzazepin-3-yl]-2-methylpropanoic acid trifluoroacetate (Preparation 156) (60 mg), (2R)-2-amino-1-propanol (11 mg, 0.15 mmol), HATU (55 mg, 0.14 mmol) and DIPEA (46 μl, 0.26 mmol) in DMF (3 ml) was stirred at room temperature overnight. The reaction mixture was diluted with ethyl acetate (10 ml). The mixture was washed with saturated sodium hydrogen carbonate, water and brine. The organic phase was dried and evaporated. The residue was chromatographed (methanol/DCM, 0-5% methanol). The product was dissolved in ethyl acetate (5 ml) and treated with hydrogen chloride in diethyl ether (1M, 0.3 ml). The solvent was evaporated and the residue triturated with diethyl ether to give 247-(5-{3-cyano-4-[(1-methylethyl)oxy]phenyl}-1,2,4-oxadiazol-3-yl)-1,2,4,5-tetrahydro-3H-3-benzazepin-3-yl]-N-(1R)-2-hydroxy-1-methylethyl]-2-methylpropanamide hydrochloride as a colourless solid (39 mg).

LCMS (Method formate): Retention time 0.94 min, MH⁺=518

### Example 164

### (2R)-3-[7-(5-{3-cyano-4-[(1-methylethyl)oxy]phenyl}-1,2,4-oxadiazol-3-yl)-1,2,4,5-tetrahydro-3H-3-benzazepin-3-yl]-2-hydroxy-N-(2-hydroxyethyl)propanamide hydrochloride

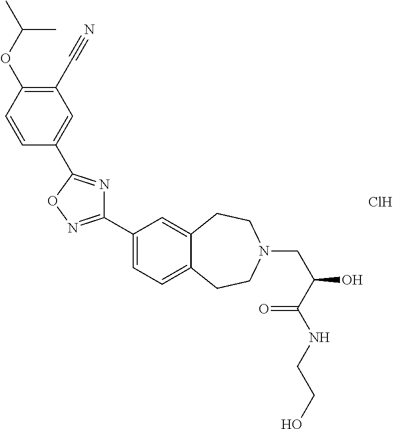

A mixture of (2R)-3-[7-(5-{3-cyano-4-[(1-methylethyl)oxy]phenyl}-1,2,4-oxadiazol-3-yl)-1,2,4,5-tetrahydro-3H-3-benzazepin-3-yl]-2-hydroxypropanoic acid (Preparation 160) (70 mg, 0.15 mmol), ethanolamine (10 mg, 0.16 mmol), HATU (64 mg, 0.17 mmol) and DIPEA (53 μl, 0.30 mmol) in DMF (3 ml) was stirred at room temperature overnight. The reaction mixture was diluted with ethyl acetate (10 ml). The mixture was washed with saturated sodium hydrogen carbonate, water and brine. The organic phase was dried and evaporated. The residue was chromatographed (methanol/DCM, 0-5% methanol). The product was dissolved in ethyl acetate (5 ml) and treated with hydrogen chloride in diethyl ether (1M, 0.3 ml). The solvent was evaporated and the residue triturated with diethyl ether to give (2R)-3-[7-(5-{3-cyano-4-[(1-methylethyl)oxy]phenyl}-1,2,4-oxadiazol-3-yl)-1,2,4,5-tetrahydro-3H-3-benzazepin-3-yl]-2-hydroxy-N-(2-hydroxyethyl)propanamide hydrochloride as a colourless solid (46 mg).

LCMS (Method formate): Retention time 0.86 min, MH⁺=506

### Example 165

### (2R)-3-[7-(5-{3-cyano-4-[(1-methylethyl)oxy]phenyl}-1,2,4-oxadiazol-3-yl)-1,2,4,5-tetrahydro-3H-3-benzazepin-3-yl]-2-hydroxy-N-(1R)-2-hydroxy-1-methylethyl]propanamide hydrochloride

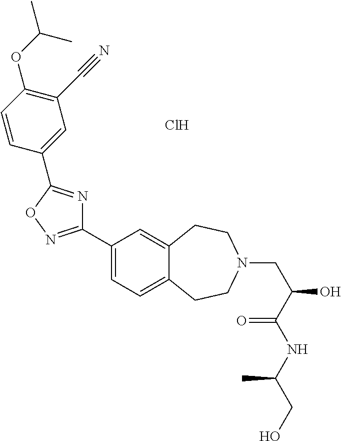

A mixture of (2R)-3-[7-(5-{3-cyano-4-[(1-methylethyl)oxy]phenyl}-1,2,4-oxadiazol-3-yl)-1,2,4,5-tetrahydro-3H-3-benzazepin-3-yl]-2-hydroxypropanoic acid (Preparation 160) (70 mg, 0.15 mmol), (2R)-2-amino-1-propanol (13 mg, 0.17 mmol), HATU (64 mg, 0.17 mmol) and DIPEA (53 μl, 0.30 mmol) in DMF (3 ml) was stirred at room temperature overnight. The reaction mixture was diluted with ethyl acetate (10 ml). The mixture was washed with saturated sodium hydrogen carbonate, water and brine. The organic phase was dried and evaporated. The residue was chromatographed (methanol/DCM, 0-10%). The residue was dissolved in ethyl acetate (5 ml) and treated with hydrogen chloride in diethyl ether (1M, 0.3 ml). The solvent was evaporated and the residue triturated with diethyl ether to give (2R)-3-[7-(5-{3-cyano-4-[(1-methylethyl)oxy]phenyl}-1,2,4-oxadiazol-3-yl)-1,2,4,5-tetrahydro-3H-3-benzazepin-3-yl]-2-hydroxy-N-(1R)-2-hydroxy-1-methylethyl]propanamide hydrochloride as a colourless solid (45 mg).

LCMS (Method formate): Retention time 0.87 min, MH⁺=520

### Example 166

### 5-(3-{2-[(2R)-2,3-dihydroxypropyl]-1,2,3,4-tetrahydro-6-isoquinolinyl}-1,2,4-oxadiazol-5-yl)-2-[(1-methylethyl)oxy]-3-pyridinecarbonitrile

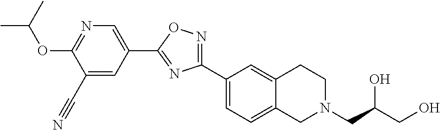

Sodium triacetoxyborohydride (334 mg, 1.6 mmol) was added portionwise to a stirred supension of 2-[(1-methylethyl)oxy]-5-[3-(1,2,3,4-tetrahydro-6-isoquinolinyl)-1,2,4-oxadiazol-5-yl]-3-pyridinecarbonitrile trifluoroacetate (Preparation 162) (150 mg, 0.31 mmol) and L-glyceraldehyde (142 mg, 1.6 mmol) in dry DCM (5 ml) and methanol (1 ml). The reaction mixture was stirred at room temperature for 24 h. Saturated sodium hydrogen carbonate (5 ml) was added and the mixture stirred vigorously for 30 min. The organic phase was separated. The aqueous phase was extracted with DCM (2×10 ml). The combined organics were dried and evaporated. Chromatography (methanol/DCM, 5-10%) gave, after trituration with diethyl ether, 5-(3-{2-[(2R)-2,3-dihydroxypropyl]-1,2,3,4-tetrahydro-6-isoquinolinyl}-1,2,4-oxadiazol-5-yl)-2-[(1-methylethyl)oxy]-3-pyridinecarbonitrile as a colourless solid (26 mg).

LCMS (Method formate): Retention time 0.84 min, MH⁺=436

### Example 167

### 5-(3-{2-[(2S)-2,3-dihydroxypropyl]-1,2,3,4-tetrahydro-6-isoquinolinyl}-1,2,4-oxadiazol-5-yl)-2-[(1-methylethyl)oxy]-3-pyridinecarbonitrile

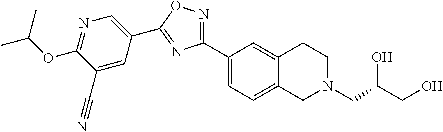

Sodium triacetoxyborohydride (334 mg, 1.6 mmol) was added portionwise to a stirred solution of 2-[(1-methylethyl)oxy]-5-[3-(1,2,3,4-tetrahydro-6-isoquinolinyl)-1,2,4-oxadiazol-5-yl]-3-pyridinecarbonitrile trifluoroacetate (Preparation 162) (150 mg, 0.31 mmol) and D-glyceraldehyde (142 mg, 1.6 mmol) in dry DCM (5 ml) and methanol (1 ml). The reaction mixture was stirred at room temperature for 24 h. Saturated sodium hydrogen carbonate (5 ml) was added carefully and the mixture stirred vigorously for 30 min. The organic phase was separated. The aqueous phase was extracted with DCM (2×10 ml). The combined organics were dried and evaporated. Chromatography (methanol/DCM, 5-10%) gave after trituration with diethyl ether 5-(3-{2-[(2S)-2,3-dihydroxypropyl]-1,2,3,4-tetrahydro-6-isoquinolinyl}-1,2,4-oxadiazol-5-yl)-2-[(1-methylethyl)oxy]-3-pyridinecarbonitrile as a colourless solid (76 mg).

LCMS (Method formate): Retention time 0.85 min, MH⁺=436

### Example 168

### 5-(3-{2-[2-hydroxy-1-(hydroxymethyl)ethyl]-1,2,3,4-tetrahydro-6-isoquinolinyl}-1,2,4-oxadiazol-5-yl)-2-[(1-methylethyl)oxy]-3-pyridinecarbonitrile hydrochloride

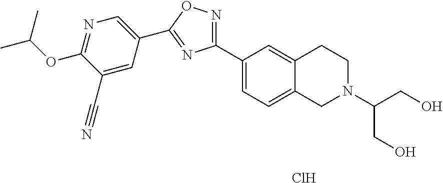

A mixture of 5-{3-[2-(2,2-dimethyl-1,3-dioxan-5-yl)-1,2,3,4-tetrahydro-6-isoquinolinyl]-1,2,4-oxadiazol-5-yl}-2-[(1-methylethyl)oxy]-3-pyridinecarbonitrile (Preparation 173) (150 mg, 0.32 mmol), THF (2 ml) and hydrochloric acid (2M, 2 ml) was stirred at room temperature for 24 h. Saturated sodium hydrogen carbonate (5 ml) was added. The mixture was extracted with ethyl acetate (3×10 ml). The combined extracts were washed with water and brine, dried and evaporated. The residue was purified by chromatography (methanol/DCM, 5-10%). The product fractions were evaporated and redissolved in ethyl acetate (2 ml). The solution was treated with hydrogen chloride in diethyl ether (1M, 0.5 ml). The solvent was evaporated and the residue triturated with diethyl ether to give 5-(3-{2-[2-hydroxy-1-(hydroxymethyl)ethyl]-1,2,3,4-tetrahydro-6-isoquinolinyl}-1,2,4-oxadiazol-5-yl)-2-[(1-methylethyl)oxy]-3-pyridinecarbonitrile hydrochloride as a pale yellow solid (90 mg).

LCMS (Method formate): Retention time 0.88 min, MH⁺=436

### Example 169

### 2-[6-(5-{3-cyano-4-[(1-methylethyl)oxy]phenyl}-1,2,4-oxadiazol-3-yl)-3,4-dihydro-2(1H)-isoquinolinyl]-N-[(1S)-2-hydroxy-1-methylethyl]acetamide

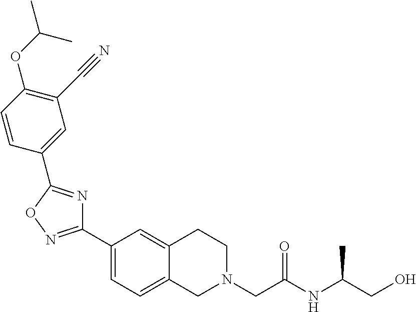

A mixture of [6-(5-{3-cyano-4-[(1-methylethyl)oxy]phenyl}-1,2,4-oxadiazol-3-yl)-3,4-dihydro-2(1H)-isoquinolinyl]acetic acid trifluoroacetate (Preparation 174) (250 mg, 0.47 mmol), N-ethylmorpholine (119 μl, 0.94 mmol), HATU (214 mg, 0.56 mmol) and L-alaninol (42 mg, 0.56 mmol) in DMF (3 ml) was stirred at room temperature overnight. The reaction mixture was diluted with ethyl acetate (15 ml). The mixture was washed with saturated sodium hydrogen carbonate (10 ml), water (10 ml) and brine (10 ml). The organic phase was dried and evaporated. Chromatography (methanol/DCM, 5-10%) gave 2-[6-(5-{3-cyano-4-[(1-methylethyl)oxy]phenyl}-1,2,4-oxadiazol-3-yl)-3,4-dihydro-2(1H)-isoquinolinyl]-N-[(1S)-2-hydroxy-1-methylethyl]acetamide as a colourless solid (128 mg).

LCMS (Method formate): Retention time 0.87 min, MH⁺=476

### Example 170

### 2-[6-(5-{5-cyano-6-[(1-methylethyl)oxy]-3-pyridinyl}-1,2,4-oxadiazol-3-yl)-5-methyl-3,4-dihydro-2(1H)-isoquinolinyl]-N-[(1S)-2-hydroxy-1-methylethyl]acetamide hydrochloride

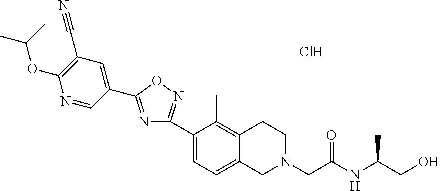

A mixture of [6-(5-{5-cyano-6-[(1-methylethyl)oxy]-3-pyridinyl}-1,2,4-oxadiazol-3-yl)-5-methyl-3,4-dihydro-2(1H)-isoquinolinyl]acetic acid trifluororacetate (Preparation 176) (180 mg, 0.33 mmol), N-ethylmorpholine (83 μl, 0.66 mmol), HATU (150 mg, 0.5 mmol) and L-alaninol (38 mg, 0.51 mmol) in DMF (5 ml) was stirred at room temperature overnight. The reaction mixture was diluted with saturated sodium hydrogen carbonate (15 ml) and extracted with ethyl acetate (3×10 ml). The combined organics were washed with water and brine, dried and evaporated. The residue was chromatographed (methanol/DCM, 0-5%). The combined product fractions were treated with hydrogen chloride in diethyl ether (1M, 1 ml). The solvent was evaporated and the residue triturated with diethyl ether to give 2-[6-(5-{5-cyano-6-[(1-methylethyl)oxy}-3-pyridinyl]-1,2,4-oxadiazol-3-yl)-5-methyl-3,4-dihydro-2(1H)-isoquinolinyl]-N-[(1S)-2-hydroxy-1-methylethyl]acetamide hydrochloride as a colourless solid (110 mg).

LCMS (Method formate): Retention time 0.87 min, MH⁺=491

### Example 171

### 5-(3-{3-[(2R)-2,3-dihydroxypropyl]-2,3,4,5-tetrahydro-1H-3-benzazepin-7-yl}-1,2,4-oxadiazol-5-yl)-2-[(1-methylethyl)amino]-3-pyridinecarbonitrile hydrochloride

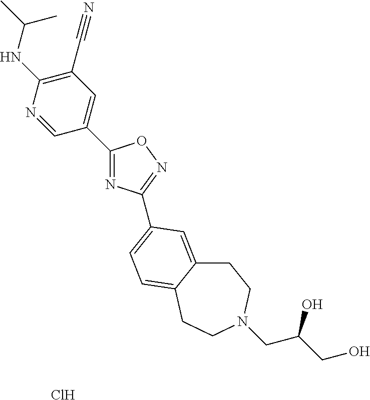

Sodium triacetoxyborohydride (1.06 g, 5 mmol) was added portionwise to a stirred solution of 2-[(1-methylethyl)amino]-5-[3-(2,3,4,5-tetrahydro-1H-3-benzazepin-7-yl)-1,2,4-oxadiazol-5-yl]-3-pyridinecarbonitrile hydrochloride (Preparation 120) (411 mg, 1 mmol) and L-glyceraldehyde (270 mg, 3 mmol) in DCM (8 ml) and methanol (2 ml). The reaction mixture was stirred at room temperature overnight. Saturated sodium hydrogen carbonate (20 ml) was added and the mixture stirred for 15 min. The organic phase was separated. The aqueous phase was extracted with DCM (10 ml) and the combined organics were dried and evaporated. The residue was chromatographed (methanol/DCM, 0-6%). The combined product fractions were treated with hydrogen chloride in diethyl ether (1M, 2 ml). The solvent was evaporated and the residue triturated with diethyl ether to give 5-(3-{3-[(2R)-2,3-dihydroxypropyl]-2,3,4,5-tetrahydro-1H-3-benzazepin-7-yl}-1,2,4-oxadiazol-5-yl)-2-[(1-methylethyl)amino]-3-pyridinecarbonitrile hydrochloride as a colourless solid (259 mg).

LCMS (Method formate): Retention time 0.79 min, MH⁺=449

### Example 172

### 5-(3-[34(2R)-2,3-dihydroxypropyl]-6-methyl-2,3,4,5-tetrahydro-1H-3-benzazepin-7-yl}-1,2,4-oxadiazol-5-yl)-2-[(1-methylethyl)amino]-3-pyridinecarbonitrile hydrochloride

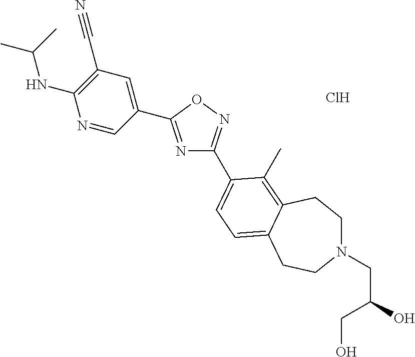

Sodium triacetoxyborohydride (748 mg, 3.5 mmol) was added portionwise to a stirred solution of 2-[(1-methylethyl)amino]-5-[3-(6-methyl-2,3,4,5-tetrahydro-1H-3-benzazepin-7-yl)-1,2,4-oxadiazol-5-yl]-3-pyridinecarbonitrile hydrochloride (Preparation 178) (300 mg, 0.71 mmol) and L-glyceraldehyde (191 mg, 2.1 mmol) in DCM (8 ml) and methanol (2 ml). The reaction mixture was stirred at room temperature overnight. Saturated sodium hydrogen carbonate (20 ml) was added and the mixture stirred for 15 min. The organic phase was separated. The aqueous phase was extracted with DCM (10 ml). The combined organics were dried and evaporated. The residue was chromatographed (methanol/DCM, 0-6%). The combined product fractions were treated with hydrogen chloride in diethyl ether (1M, 2 ml). The solvent was evaporated and the residue triturated with diethyl ether to give 5-(3-{3-[(2R)-2,3-dihydroxypropyl]-6-methyl-2,3,4,5-tetrahydro-1H-3-benzazepin-7-yl}-1,2,4-oxadiazol-5-yl)-2-[(1-methylethyl)amino]-3-pyridinecarbonitrile hydrochloride as a colourless solid (89 mg).

LCMS (Method formate): Retention time 0.83 min, MH⁺=463

### Example 173

### 5-(3-{3-[(2S)-2,3-dihydroxypropyl]-6-methyl-2,3,4,5-tetrahydro-1H-3-benzazepin-7-yl}-1,2,4-oxadiazol-5-yl)-2-[(1-methylethyl)amino]-3-pyridinecarbonitrile hydrochloride

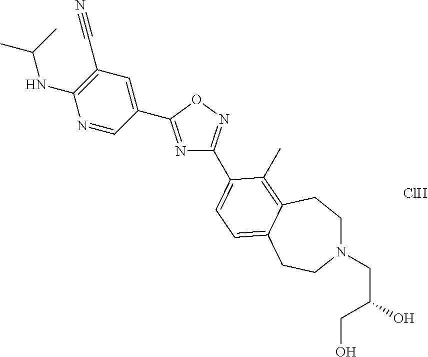

Sodium triacetoxyborohydride (748 mg, 3.5 mmol) was added portionwise to a stirred solution of 2-[(1-methylethyl)amino]-5-[3-(6-methyl-2,3,4,5-tetrahydro-1H-3-benzazepin-7-yl)-1,2,4-oxadiazol-5-yl]-3-pyridinecarbonitrile hydrochloride (Preparation 178) (300 mg, 0.71 mmol) and D-glyceraldehyde (191 mg, 2.1 mmol) in DCM (8 ml) and methanol (2 ml). The reaction mixture was stirred at room temperature overnight. Saturated sodium hydrogen carbonate (20 ml) was added and the mixture stirred for 15 min. The organic phase was separated. The aqueous phase was extracted with DCM (10 ml). The combined organics were dried and evaporated. The residue was chromatographed (methanol/DCM, 0-6%). The combined product fractions were treated with hydrogen chloride in diethyl ether (1M, 2 ml). The sample purified by MDAP (x2, Method formate) and the product further purified by chromatography (methanol/DCM, 4-8%). The combined product fractions were treated with hydrogen chloride in diethyl ether (1M, 2 ml). The solvent was evaporated and the residue triturated with diethyl ether to give 5-(3-{3-[(2S)-2,3-dihydroxypropyl]-6-methyl-2,3,4,5-tetrahydro-1H-3-benzazepin-7-yl}-1,2,4-oxadiazol-5-yl)-2-[(1-methylethyl)amino]-3-pyridinecarbonitrile hydrochloride as a colourless solid.

LCMS (Method formate): Retention time 0.88 min, MH⁺=463

### Example 174

### 5-(3-{2-[(2R)-2,3-dihydroxypropyl]-5-methyl-1,2,3,4-tetrahydro-6-isoquinolinyl}-1,2,4-oxadiazol-5-yl)-2-[(1-methylethyl)amino]-3-pyridinecarbonitrile hydrochloride

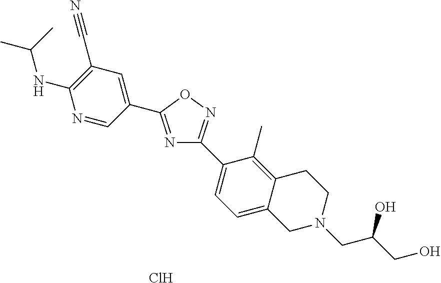

Sodium triacetoxyborohydride (516 mg, 2.43 mmol) was added portionwise to a stirred solution of 2-[(1-methylethyl)amino]-5-[3-(5-methyl-1,2,3,4-tetrahydro-6-isoquinolinyl)-1,2,4-oxadiazol-5-yl]-3-pyridinecarbonitrile hydrochloride (Preparation 180) (200 mg, 0.49 mmol) and L-glyceraldehyde (132 mg, 1.5 mmol) in DCM (8 ml) and methanol (2 ml). The reaction mixture was stirred at room temperature overnight. Saturated sodium hydrogen carbonate (20 ml) was added and the mixture stirred for 15 min. The organic phase was separated. The aqueous phase was extracted with DCM (10 ml). The combined organics were dried and evaporated. The residue was chromatographed (methanol/DCM, 0-6%). The combined product fractions were treated with hydrogen chloride in diethyl ether (1M, 2 ml). The solvent was evaporated and the residue triturated with diethyl ether to give 5-(3-{2-[(2R)-2,3-dihydroxypropyl]-5-methyl-1,2,3,4-tetrahydro-6-isoquinolinyl}-1,2,4-oxadiazol-5-yl)-2-[(1-methylethyl)amino]-3-pyridinecarbonitrile hydrochloride as a colourless solid (89 mg).

LCMS (Method formate): Retention time 0.81 min, MH⁺=449

### Example 175

### 5-(3-{3-[2-hydroxy-1-(hydroxymethyl)ethyl]-6-methyl-2,3,4,5-tetrahydro-1H-3-benzazepin-7-yl}-1,2,4-oxadiazol-5-yl)-2-[(1-methylethyl)amino]-3-pyridinecarbonitrile hydrochloride

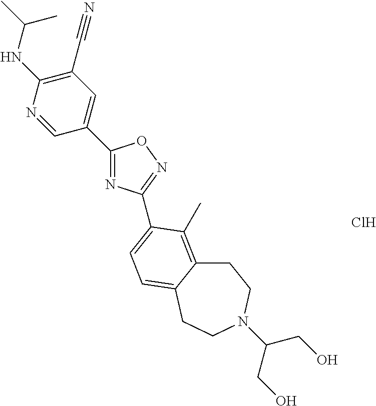

Sodium triacetoxyborohydride (748 mg, 3.5 mmol) was added portionwise to a stirred solution of 2-[(1-methylethyl)amino]-5-[3-(6-methyl-2,3,4,5-tetrahydro-1H-3-benzazepin-7-yl)-1,2,4-oxadiazol-5-yl]-3-pyridinecarbonitrile hydrochloride (Preparation 178) (300 mg, 0.71 mmol) and 2,2-dimethyl-1,3-dioxan-5-one (253 μl, 2.1 mmol) in DCM (10 ml). The reaction mixture was stirred at room temperature overnight. Saturated sodium hydrogen carbonate (20 ml) was added and the mixture stirred for 15 min. The organic phase was separated. The aqueous phase was extracted with DCM (2×10 ml). The combined organics were dried and evaporated. The residue was dissolved in THF (5 ml) and the solution treated with hydrochloric acid (2M, 5 ml). The reaction mixture was allowed to stand at room temperature over the weekend. Saturated sodium hydrogen carbonate (10 ml) was added and the mixture stirred for 10 min. The mixture was extracted with DCM (3×10 ml). The combined organics were washed with water, dried, and evaporated. The residue was chromatographed (methanol/DCM, 0-8%). The combined product fractions were treated with hydrogen chloride in diethyl ether (1M, 5 ml). The solvent was evaporated and the residue triturated with diethyl ether to give 5-(3-{3-[2-hydroxy-1-(hydroxymethyl)ethyl]-6-methyl-2,3,4,5-tetrahydro-1H-3-benzazepin-7-yl}-1,2,4-oxadiazol-5-yl)-2-[(1-methylethyl)amino]-3-pyridinecarbonitrile hydrochloride as a colourless solid (181 mg).

LCMS (Method formate): Retention time 0.89 min, MH⁺=463

### Example 176

### 5-(3-{2-[2-hydroxy-1-(hydroxymethyl)ethyl]-5-methyl-1,2,3,4-tetrahydro-6-isoquinolinyl}-1,2,4-oxadiazol-5-yl)-2-[(1-methylethyl)amino]-3-pyridinecarbonitrile hydrochloride

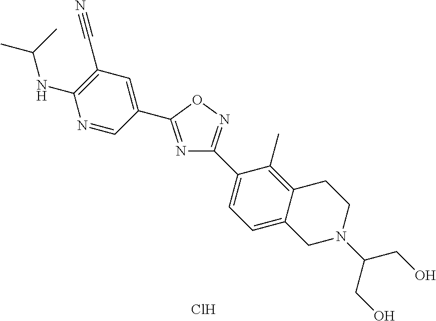

Sodium triacetoxyborohydride (516 mg, 2.4 mmol) was added portionwise to a stirred solution of 2-[(1-methylethyl)amino]-5-[3-(5-methyl-1,2,3,4-tetrahydro-6-isoquinolinyl)-1,2,4-oxadiazol-5-yl]-3-pyridinecarbonitrile hydrochloride (Preparation 180) (200 mg, 0.49 mmol) and 2,2-dimethyl-1,3-dioxan-5-one (174 μl, 1.5 mmol) in DCM (10 ml). The reaction mixture was stirred at room temperature overnight. Saturated sodium hydrogen carbonate (20 ml) was added and the mixture stirred for 15 min. The organic phase was separated. The aqueous phase was extracted with DCM (2×10 ml). The combined organics were dried and evaporated. The residue was dissolved in THF (5 ml) and the solution treated with hydrochloric acid (2M, 5 ml). The reaction mixture was allowed to stand at room temperature over the weekend. Saturated sodium hydrogen carbonate (10 ml) was added and the mixture stirred for 10 min. The mixture was extracted with DCM (3×10 ml), the combined organics were washed with water, dried, and evaporated. The residue was chromatographed (methanol/DCM, 0-8%). The combined product fractions were treated with hydrogen chloride in diethyl ether (1M, 2 ml). The solvent was evaporated and the residue triturated with diethyl ether to give 5-(3-{2-[2-hydroxy-1-(hydroxymethyl)ethyl]-5-methyl-1,2,3,4-tetrahydro-6-isoquinolinyl}-1,2,4-oxadiazol-5-yl)-2-[(1-methylethyl)amino]-3-pyridinecarbonitrile hydrochloride as a colourless solid (26 mg).

LCMS (Method formate): Retention time 0.87 min, MH⁺=449

### Example 177

### 5-(3-{2-[(2S)-2,3-dihydroxypropyl]-5-methyl-1,2,3,4-tetrahydro-6-isoquinolinyl}-1,2,4-oxadiazol-5-yl)-2-[(1-methylethyl)amino]-3-pyridinecarbonitrile hydrochloride

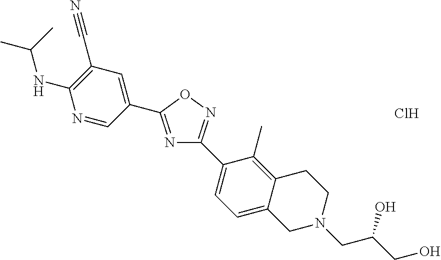

Sodium triacetoxyborohydride (516 mg, 2.4 mmol) was added portionwise to a stirred solution of 2-[(1-methylethyl)amino]-5-[3-(5-methyl-1,2,3,4-tetrahydro-6-isoquinolinyl)-1,2,4-oxadiazol-5-yl]-3-pyridinecarbonitrile hydrochloride (Preparation 180) (200 mg, 0.49 mmol) and D-glyceraldehyde (132 mg, 1.5 mmol) in DCM (8 ml) and methanol (2 ml). The reaction mixture was stirred at room temperature overnight. Saturated sodium hydrogen carbonate (20 ml) was added and the mixture stirred for 15 min. The organic phase was separated. The aqueous phase was extracted with DCM (10 ml). The combined organics were dried and evaporated. The residue was chromatographed (methanol/DCM, 0-6%). The combined product fractions were treated with hydrogen chloride in diethyl ether (1M, 2 ml). The solvent was evaporated and the residue triturated with diethyl ether to give 5-(3-{2-[(2S)-2,3-dihydroxypropyl]-5-methyl-1,2,3,4-tetrahydro-6-isoquinolinyl}-1,2,4-oxadiazol-5-yl)-2-[(1-methylethyl)amino]-3-pyridinecarbonitrile hydrochloride as a colourless solid (77 mg).

LCMS (Method formate): Retention time 0.80 min, MH⁺=449

### Example 178

### 5-(3-{2-[(2R)-2,3-dihydroxypropyl]-1,2,3,4-tetrahydro-5-isoquinolinyl}-1,2,4-oxadiazol-5-yl)-2-[(1-methylethyl)oxy]benzonitrile hydrochloride

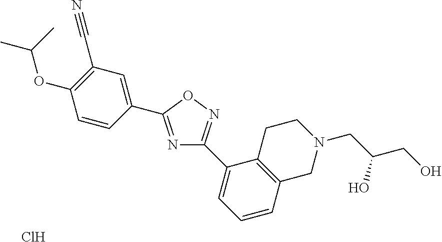

To a stirred solution of 2-[(1-methylethyl)oxy]-5-[3-(1,2,3,4-tetrahydro-5-isoquinolinyl)-1,2,4-oxadiazol-5-yl]benzonitrile trifluoroacetate (Preparation 91) (400 mg, 0.84 mmol) in DCM (7 ml) was added D-glyceraldhyde (380 mg, 4.2 mmol) in methanol (4 ml). The solution was stirred for 10 min, after which time sodium triacetoxyborohydride (893 mg, 4.2 mmol) was slowly added and the solution was stirred for 15 h. Saturated sodium hydrogen carbonate (5 ml) was carefully added to the reaction mixture, which was partitioned between water (30 ml) and DCM (30 ml), and the aqueous layer was re-extracted with DCM (2×20 ml). The resultant organic phases were dried (hydrophbic frit) and evaporated under reduced pressure. The resultant solid was dissolved in methanol/DMSO (1:1) and the precipitate filtered off. The residue and filtrate were then dissolved in DCM and the solvents evaporated under a stream of nitrogen over 24 h. The resultant solid was dissolved in DCM and purified by chromatography on a silica cartridge (10 g) eluting with a gradient of methanol/DCM (0-5%). The appropriate fractions were combined and evaporated under vacuum. The resultant oil was dissolved in methanol and hydrogen chloride in diethyl ether (1 M, 0.5 ml) was added to the solution, the solution was allowed to stir for 10 min, diluted with diethyl ether (ca 10 ml) was added to the solution. The off-white solid precipitate was isolated by filtration and dried to give 5-(3-{2-[(2R)-2,3-dihydroxypropyl]-1,2,3,4-tetrahydro-5-isoquinolinyl}-1,2,4-oxadiazol-5-yl)-2-[(1-methylethyl)oxy]benzonitrile hydrochloride as a white solid (75 mg).

LCMS (Method formate): Retention time 0.89 min, MH⁺=435

### Example 179

### 5-(3-{2-[(2R)-2,3-dihydroxypropyl]-1,2,3,4-tetrahydro-5-isoquinolinyl}-1,2,4-oxadiazol-5-yl)-2-[(1-methylethyl)oxy]benzonitrile hydrochloride

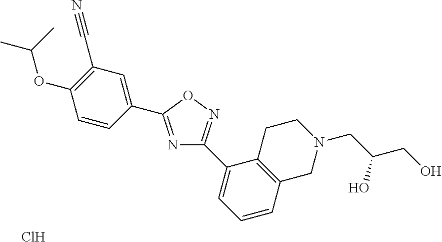

To a stirred solution of 2-[(1-methylethyl)oxy]-5-[3-(1,2,3,4-tetrahydro-5-isoquinolinyl)-1,2,4-oxadiazol-5-yl]benzonitrile trifluoroacetate (Preparation 91) (400 mg, 0.84 mmol) in DCM (7 ml) was added (2S)-2,3-dihydroxypropanal (380 mg, 4.2 mmol) in methanol (4 ml). The solution was stirred for 10 min, sodium triacetoxyborohydride (893 mg, 4.2 mmol) was slowly added and the reaction was stirred for 15 h. To the reaction mixture was slowly added ˜5 ml of saturated sodium hydrogen carbonate. The reaction mixture was extracted using water (30 ml) and ethyl acetate (3×30 ml). The organic phases were dried (hydrophobic frit) and the resultant solution evaporated under reduced pressure. The resultant solid was dissolved in methanol/DMSO (1:1, 4 ml) in and purified by Mass Directed AutoPrep (x4, Method formate). The solvent was evaporated under vacuum, the white solid dissolved in DCM and loaded onto a silica cartridge (10 g), eluting with a gradient of methanol/DCM (0-5%). The appropriate fractions were combined and evaporated under vacuum. The resulting solid was dissolved in methanol (ca 2 ml) and treated with hydrogen chloride in diethyl ether (1M, 1 ml). The solution was stirred for 10 min, diluted with diethyl ether (ca 30 ml) and the precipitated solid isolated by filtration. Drying gave 5-(3-{2-[(2R)-2,3-dihydroxypropyl]-1,2,3,4-tetrahydro-5-isoquinolinyl}-1,2,4-oxadiazol-5-yl)-2-[(1-methylethyl)oxy]benzonitrile hydrochloride as a white solid (96 mg).

LCMS (Method formate): Retention time 0.85 min, MH⁺=435

### Example 180

### 5-(3-{2-[2-hydroxy-1-(hydroxymethyl)ethyl]-1,2,3,4-tetrahydro-5-isoquinolinyl}-1,2,4-oxadiazol-5-yl)-2-[(1-methylethyl)oxy]benzonitrile hydrochloride

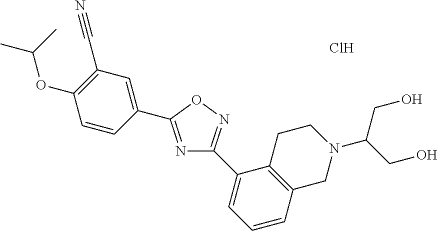

To a stirred solution of 5-{3-[2-(2,2-dimethyl-1,3-dioxan-5-yl)-1,2,3,4-tetrahydro-5-isoquinolinyl]-1,2,4-oxadiazol-5-yl}-2-[(1-methylethyl)oxy]benzonitrile (Preparation 182) (463 mg) in THF (15 ml) was added hydrochloric acid (2M, 15 ml) and the solution was stirred overnight. The solvents were evaporated under reduced pressure, water added to the residue and the mixture neutralised by addition of sodium hydrogen carbonate solution. The solid was isolated by filtration and the solid dissolved in methanol/DCM (10%). The resultant solution was dried (hydrophobic frit) and concentrated under reduced pressure. The resultant solid was dissolved in methanol (15 ml) and treated with hydrogen chloride in diethyl ether (1M, 1 ml). The solution was stirred for 10 min and diluted with diethyl ether (˜30 ml). The resulting precipitate was isolated by filtration and dried to give 5-(3-{2-[2-hydroxy-1-(hydroxymethyl)ethyl]-1,2,3,4-tetrahydro-5-isoquinolinyl}-1,2,4-oxadiazol-5-yl)-2-[(1-methylethyl)oxy]benzonitrile hydrochloride as a white solid (233 mg).

LCMS (Method formate): Retention time 0.84 min, MH⁺=435

### Example 181

### 5-(5-{3-[(2R)-2,3-dihydroxypropyl]-2,3,4,5-tetrahydro-1H-3-benzazepin-7-yl}-1,3,4-thiadiazol-2-yl)-2-[(1-methylethyl)oxy]benzonitrile hydrochloride

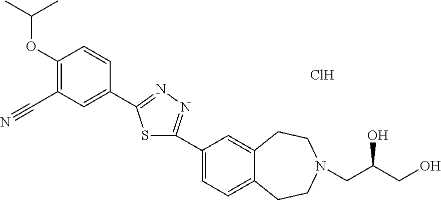

L-Glyceraldehyde (153 mg, 1.7 mmol) was dissolved in methanol (2 ml) and added to 2-[(1-methylethyl)oxy]-5-[3-(2,3,4,5-tetrahydro-1H-3-benzazepin-7-yl)-1,3,4-thiadiazol-2-yl]benzonitrile trifluoroacetate (Preparation 47) (171 mg, 0.34 mmol) in DCM (10 ml). Sodium triacetoxyborohydride (359 mg, 1.7 mmol) was added to the mixture which was stirred overnight at room temperature. The reaction mixture was partitioned between ethyl acetate (50 ml) and saturated sodium hydrogen carbonate (30 ml). The organic phase was washed with brine (30 ml), dried (MgSO₄), filtered and the solvent evaporated. The residue was chromatographed on a silica column (20 g) eluting with a methanol/DCM gradient (4-6%). The product was treated with hydrogen chloride in diethyl ether (1M, 1 ml) to give 5-(5-{3-[(2R)-2,3-dihydroxypropyl]-2,3,4,5-tetrahydro-1H-3-benzazepin-7-yl}-1,3,4-thiadiazol-2-yl)-2-[(1-methylethyl)oxy]benzonitrile hydrochloride (105 mg) as a white solid.

LCMS (Method HpH): Retention time 1.08 min, MH⁺=465

### Example 182

### 5-(5-{3-[(2S)-2,3-dihydroxypropyl]-2,3,4,5-tetrahydro-1H-3-benzazepin-7-yl}-1,3,4-thiadiazol-2-yl)-2-[(1-methylethyl)oxy]benzonitrile hydrochloride

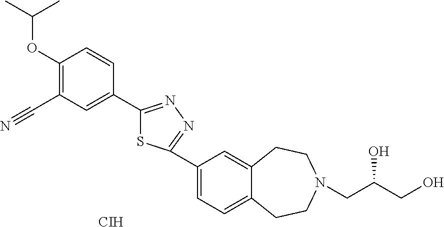

D-glyceraldehyde (197 mg, 2.2 mmol) was dissolved in methanol (2 ml) and added to 2-[(1-methylethyl)oxy]-5-[3-(2,3,4,5-tetrahydro-1H-3-benzazepin-7-yl)-1,3,4-thiadiazol-2-yl]benzonitri2-[(1-methylethyl)oxy]-5-[3-(2,3,4,5-tetrahydro-1H-3-benzazepin-7-yl)-1,3,4-thiadiazol-2-yl]benzonitrile trifluoroacetate (Preparation 47) (171 mg, 0.34 mmol) in DCM (10 ml). Sodium triacetoxyborohydride (464 mg, 2.2 mmol) was added to the mixture which was stirred overnight at room temperature. The reaction mixture was partitioned between ethyl acetate (50 ml) and saturated sodium hydrogen carbonate (30 ml). The organic phase was washed with brine (30 ml), dried (MgSO₄), filtered and the solvent evaporated. The residue was chromatographed on a silica cartridge (20 g) eluting with a methanol/DCM gradient (4-6%) to give 5-(5-{3-[(2S)-2,3-dihydroxypropyl]-2,3,4,5-tetrahydro-1H-3-benzazepin-7-yl}-1,3,4-thiadiazol-2-yl)-2-[(1-methylethyl)oxy]benzonitrile.

LCMS (Method HpH): Retention time 1.08 min, MH⁺=465

This was converted to the hydrochloride salt by treating the product with hydrogen chloride in diethyl ether (1M, 1 ml) to 5-(5-{3-[(2S)-2,3-dihydroxypropyl]-2,3,4,5-tetrahydro-1H-3-benzazepin-7-yl}-1,3,4-thiadiazol-2-yl)-2-[(1-methylethyl)oxy]benzonitrile hydrochloride as a white solid (91 mg)

### Example 183

### 2-[7-(5-{3-cyano-4-[(1-methylethyl)oxy]phenyl}-1,2,4-oxadiazol-3-yl)-3,4-dihydro-2(1H)-isoquinolinyl]-N-[(1S)-2-hydroxy-1-methylethyl]acetamide hydrochloride

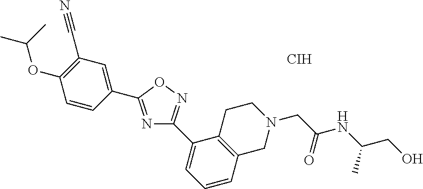

To a stirred solution of [5-(5-{3-cyano-4-[(1-methylethyl)oxy]phenyl}-1,2,4-oxadiazol-3-yl)-3,4-dihydro-2(1H)-isoquinolinyl]acetic acid trifluoroacetate (Preparation 183) (250 mg, 0.6 mmol) in acetonitrile (2 ml), N-methyl-2-pyrrolidine (4 ml) and DMSO (2 ml) was added HATU (227 mg, 0.60 mmol), DIPEA (0.31 ml, 1.8 mmol) and (2S)-2-amino-1-propanol (0.07 ml, 0.90 mmol). The solution was stirred overnight, saturated sodium hydrogen carbonate (30 ml) was added to the reaction mixture and the mixture was extracted with ethyl acetate (3×30 ml). The organic phase was dried (hydrophobic frit) and the resultant solution was concentrated under reduced pressure. The resultant oil was dissolved in methanol (2 ml) and a further methanol/DMSO (1:1, 4 ml) and purified in 5 equal portions by MDAP (Method formate). The solvent was evaporated under vacuum to give 2-[7-(5-{3-cyano-4-[(1-methylethyl)oxy]phenyl}-1,2,4-oxadiazol-3-yl)-3,4-dihydro-2(1H)-isoquinolinyl]-N-[(1S)-2-hydroxy-1-methylethyl]acetamide in 2 batches. The first batch was dissolved in methanol (˜2 ml) and hydrogen chloride in diethyl ether (1M, 1 ml) was added to the solution, stirred for 10 min, and diethyl ether (10 ml) .added to the solution. The solution was evaporated under reduced pressure, the resulting solid was dissolved in methanol (˜2 ml) and hydrogen chloride in diethyl ether (1M, 2.5 ml) was added, stirred for 10 min and evaporated under reduced pressure to give a colourless oil. The second batch was dissolved in methanol (˜2 ml) in hydrogen chloride in diethyl ether (1M, 2.5 ml) was added and the solution stirred for 10 min. The solution was diluted with diethyl ether (20 ml) and evaporated under reduced pressure. The 2 batches were combined to give 2-[7-(5-{3-cyano-4-[(1-methylethyl)oxy]phenyl}-1,2,4-oxadiazol-3-yl)-3,4-dihydro-2(1H)-isoquinolinyl]-N-[(1S)-2-hydroxy-1-methylethyl]acetamide hydrochloride (30 mg) as a colourless oil.

LCMS (Method formate): Retention time 0.87 min, MH⁺=476

### Example 184

### 5-(5-{2-[2-hydroxy-1-(hydroxymethyl)ethyl]-5-methyl-1,2,3,4-tetrahydro-6-isoquinolinyl}-1,3,4-thiadiazol-2-yl)-2-[(1-methylethyl)oxy]benzonitrile hydrochloride

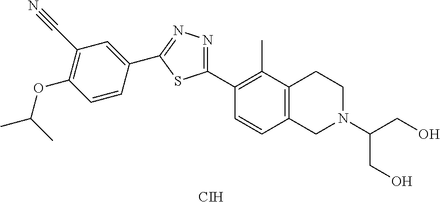

To a solution of 5-{5-[2-(2,2-dimethyl-1,3-dioxan-5-yl)-5-methyl-1,2,3,4-tetrahydro-6-isoquinolinyl]-1,3,4-thiadiazol-2-yl}-2-[(1-methylethyl)oxy]benzonitrile (Preparation 185) (824 mg, 1.6 mmol) in THF (15 ml) was added hydrochloric acid (2M, 15 ml). The resulting mixture was stirred at room temperature for 2 h, concentrated in vacuo, and coevaporated with toluene (x2). The resulting solid residue was triturated with diethyl ether and the solid isolated by filtration to give 5-(5-{2-[2-hydroxy-1-(hydroxymethyl)ethyl]-5-methyl-1,2,3,4-tetrahydro-6-isoquinolinyl}-1,3,4-thiadiazol-2-yl)-2-[(1-methylethyl)oxy]benzonitrile hydrochloride (502 mg) as a light yellow solid.

LCMS (Method formate): Retention time 0.80 min, MH⁺=465

1H NMR (D₆-DMSO): δ H 10.29 (1H, bs), 8.39 (1H, d), 8.31 (1H, dd), 7.57 (1H, d), 7.51 (1H, d), 7.28 (1H, d), 5.50 (2H, bs), 4.95 (1H, m), 4.71 (1H, m), 4.59 (1H, m), 3.90 (5H, m), 3.56 (2H, bm—partially obscured by water), 3.13 (2H, m), 2.39 (3H, s), 1.38 (6H, d).

### Example 185

### 2-[7-(5-{3-cyano-4-[(1-methylethyl)oxy]phenyl}-1,2,4-oxadiazol-3-yl)-1,2,4,5-tetrahydro-3H-3-benzazepin-3-yl]-N-(3,3,3-trifluoro-2-hydroxypropyl)acetamide

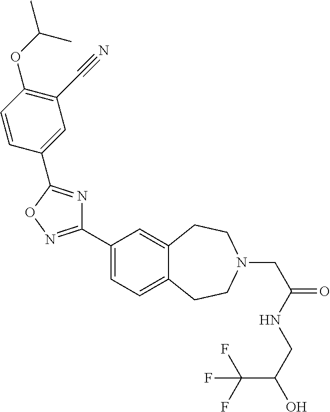

3-Amino-1,1,1-trifluoro-2-propanol (26 mg, 0.2 mmol, Fluorochem) was added to a stirred mixture of [7-(5-{3-cyano-4-[(1-methylethyl)oxy]phenyl}-1,2,4-oxadiazol-3-yl)-1,2,4,5-tetrahydro-3H-3-benzazepin-3-yl]acetic acid trifluoroacetate (Preparation 89) (100 mg, 0.18 mmol), DIPEA (64 μl, 0.36 mmol) and HATU (77 mg, 0.2 mmol) in DMF (3 ml). The reaction mixture was stirred at room temperature overnight. The reaction mixture was diluted with ethyl acetate (10 ml). The solution was washed with saturated sodium hydrogen carbonate (10 ml), water (2×20 ml) and brine (10 ml). The organic phase was dried and evaporated. The residue was chromatographed methanol/DCM (0-5%). Trituration with diethyl ether gave 2-[7-(5-{3-cyano-4-[(1-methylethyl)oxy]phenyl}-1,2,4-oxadiazol-3-yl)-1,2,4,5-tetrahydro-3H-3-benzazepin-3-yl]-N-(3,3,3-trifluoro-2-hydroxypropyl)acetamide as an off-white solid (45 mg).

LCMS (Method formate): Retention time 0.93 min, MH⁺=544

### Example 186

### 2-[7-(5-{3-cyano-4-[(1-methylethyl)oxy]phenyl}-1,2,4-oxadiazol-3-yl)-6-methyl-1,2,4,5-tetrahydro-3H-3-benzazepin-3-yl]-N-[2-hydroxy-1-(hydroxymethyl)ethyl]acetamide

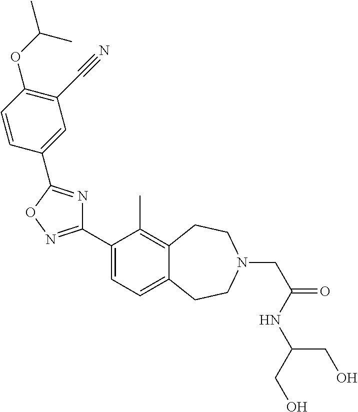

Serinol hydrochloride (25 mg, 0.2 mmol) was added to a stirred mixture of [7-(5-{3-cyano-4-[(1-methylethyl)oxy]phenyl}-1,2,4-oxadiazol-3-yl)-6-methyl-1,2,4,5-tetrahydro-3H-3-benzazepin-3-yl]acetic acid trifluoroacetate (Preparation 150) (80 mg), DIPEA (62 μl, 0.35 mmol) and HATU (75 mg, 0.2 mmol) in DMF (3 ml). The reaction mixture was stirred at room temperature overnight. The reaction mixture was diluted with ethyl acetate (10 ml). The solution was washed with saturated sodium hydrogen carbonate (10 ml), water (2×20 ml) and brine (10 ml). The organic phase was dried and evaporated. The residue was chromatographed (methanol/DCM, 0-5%). Trituration with diethyl ether gave 2-[7-(5-{3-cyano-4-[(1-methylethyl)oxy]phenyl}-1,2,4-oxadiazol-3-yl)-6-methyl-1,2,4,5-tetrahydro-3H-3-benzazepin-3-yl]-N-[2-hydroxy-1-(hydroxymethyl)ethyl)acetamide as an off-white solid (46 mg).

LCMS (Method formate): Retention time 0.85 min, MH⁺=520

### Example 187

### 5-(3-{3-[N-(2,3-dihydroxypropyl)glycyl]-6-methyl-2,3,4,5-tetrahydro-1H-3-benzazepin-7-yl}-1,2,4-oxadiazol-5-yl)-2-[(1-methylethyl)oxy]benzonitrile

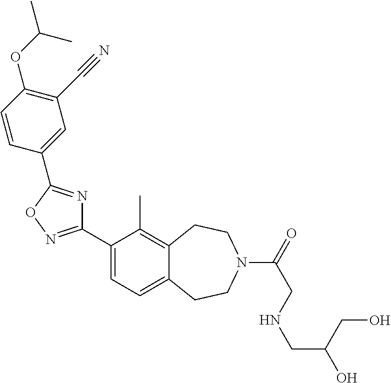

A mixture of 5-{3-[3-(bromoacetyl)-6-methyl-2,3,4,5-tetrahydro-1H-3-benzazepin-7-yl]-1,2,4-oxadiazol-5-yl}-2-[(1-methylethyl)oxy]benzonitrile (Preparation 155) (100 mg, 0.2 mmol), racemic-3-amino-1,2-propanediol (89 mg, 0.98 mmol), and potassium carbonate (68 mg, 0.49 mmol) in acetonitrile (3 ml) was stirred at room temperature overnight. The solvent was evaporated, the residue chromatographed (methanol/DCM, 0-10%) and the residue triturated with diethyl ether to give 5-(3-{3-[N-(2,3-dihydroxypropyl)glycyl]-6-methyl-2,3,4,5-tetrahydro-1H-3-benzazepin-7-yl}-1,2,4-oxadiazol-5-yl)-2-[(1-methylethyl)oxy]benzonitrile (43 mg) as a colourless solid.

LCMS (Method formate): Retention time 0.93 min, MH⁺=520

### Example 188

### 5-(3-{3-[N-(2,3-dihydroxypropyl)glycyl]-8-methyl-2,3,4,5-tetrahydro-1H-3-benzazepin-7-yl}-1,2,4-oxadiazol-5-yl)-2-[(1-methylethyl)oxy]benzonitrile

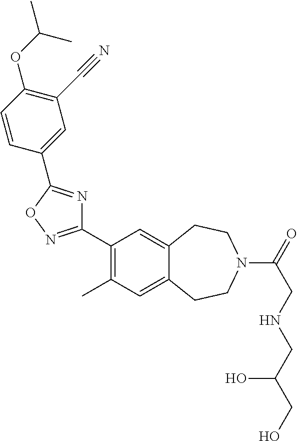

A mixture of 5-{3-[3-(bromoacetyl)-8-methyl-2,3,4,5-tetrahydro-1H-3-benzazepin-7-yl]-1,2,4-oxadiazol-5-yl}-2-[(1-methylethyl)oxy]benzonitrile (Preparation 187) (140 mg, 0.27 mmol), 3-amino-1,2-propanediol (125 mg, 1.4 mmol), and potassium carbonate (95 mg, 0.69 mmol) in acetonitrile (3 ml) was stirred at room temperature overnight. The solvent was evaporated and the residue chromatographed (methanol/DCM, 0-10%), and the residue triturated with diethyl ether to give 5-(3-{3-[N-(2,3-dihydroxypropyl)glycyl]-8-methyl-2,3,4,5-tetrahydro-1H-3-benzazepin-7-yl}-1,2,4-oxadiazol-5-yl)-2-[(1-methylethyl)oxy]benzonitrile as a colourless oil (61 mg).

LCMS (Method formate): Retention time 1.02 min, MH⁺=520

### Example 189

### 5-[3-(3-{N-[2-hydroxy-1-(hydroxymethyl)ethyl]glycyl}-6-methyl-2,3,4,5-tetrahydro-1H-3-benzazepin-7-yl)-1,2,4-oxadiazol-5-yl]-2-[(1-methylethyl)oxy]benzonitrile

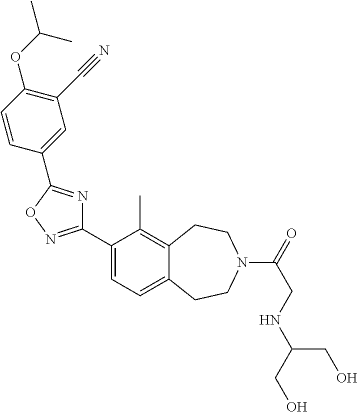

A mixture of 5-{3-[3-(bromoacetyl)-6-methyl-2,3,4,5-tetrahydro-1H-3-benzazepin-7-yl]-1,2,4-oxadiazol-5-yl}-2-[(1-methylethyl)oxy]benzonitrile (Preparation 155) (100 mg, 0.2 mmol), serinol hydrochloride (125 mg, 0.98 mmol), and potassium carbonate (136 mg, 0.98 mmol) in acetonitrile (3 ml) was stirred at room temperature overnight. The solvent was evaporated and the residue chromatographed (methanol/DCM, 0-10%). Trituration with diethyl ether gave 5-[3-(3-{N-[2-hydroxy-1-(hydroxymethyl)ethyl)glycyl}-6-methyl-2,3,4,5-tetrahydro-1H-3-benzazepin-7-yl)-1,2,4-oxadiazol-5-yl]-2-[(1-methylethyl)oxy]benzonitrile (44 mg) as a colourless solid.

LCMS (Method formate): Retention time 0.99 min, MH⁺=520

### Example 190

### 2-({2-[6-(5-{3-chloro-4-[(1-methylethyl)oxy]phenyl}-1,2,4-oxadiazol-3-yl)-5-methyl-3,4-dihydro-2(1H)-isoquinolinyl]-2-oxoethyl}amino)-1,3-propanediol hydrochloride

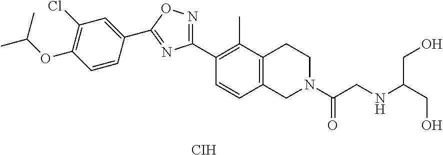

Acetonitrile (3 ml) was added to a mixture of 2-(bromoacetyl)-6-(5-{3-chloro-4-[(1-methylethyl)oxy]phenyl}-1,2,4-oxadiazol-3-yl)-5-methyl-1,2,3,4-tetrahydroisoquinoline (Preparation 132) (101 mg, 0.2 mmol) and potassium carbonate (280 mg, 2.0 mmol) at room temperature and the resulting mixture was treated with 2,2′-iminodiethanol (140 mg, 1.0 mmol) then stirred at room temperature for 2 h. 2,2′-Iminodiethanol (70 mg) and potassium carbonate (140 mg) were added and stirring continued for 3 h. The reaction was concentrated and the residue partitioned between ethyl acetate and saturated sodium hydrogen carbonate. The aqueous was extracted, the combined organic dried (MgSO₄) and concentrated in vacuo to give a residue which was purified by MDAP (Method HpH). The residue was dissolved in DCM, dried (hydrophobic frit) and the solvent was removed in vacuo. The residue was dissolved in dioxane (3 ml) and treated with hydrogen chloride in 1,4-dioxane (4M, 1 ml). The mixture was concentrated in vacuo and the residue triturated with diethyl ether. The solid was isolated by filtration to give 2-({2-[6-(5-{3-chloro-4-[(1-methylethyl)oxy]phenyl}-1,2,4-oxadiazol-3-yl)-5-methyl-3,4-dihydro-2(1H)-isoquinolinyl]-2-oxoethyl]amino)-1,3-propanediol hydrochloride (8 mg, 7%) as a white solid.

LCMS (Method HpH): Retention time 1.21 min, MH⁺=515/517

### Example 191

### 5-[3-(3-glycyl-2,3,4,5-tetrahydro-1H-3-benzazepin-7-yl)-1,2,4-oxadiazol-5-yl]-2-[(2,2,2-trifluoroethyl)oxy]benzonitrile hydrochloride

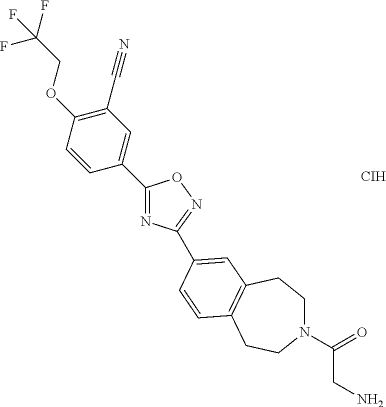

Hydrogen chloride in 1,4-dioxane (4M, 1 ml) was added to a solution of 1,1-dimethylethyl {2-[7-(5-{3-cyano-4-[(2,2,2-trifluoroethyl)oxy]phenyl}-1,2,4-oxadiazol-3-yl)-1,2,4,5-tetrahydro-3H-3-benzazepin-3-yl]-2-oxoethyl}carbamate (Preparation 188) (170 mg, 0.3 mmol) in 1,4-dioxane (1 ml). The reaction mixture was stirred at room temperature for 3 h. Ether (10 ml) was added and the mixture stirred for 20 min. The solid was isolated by filtration, washed with diethyl ether and dried to give 5-[3-(3-glycyl-2,3,4,5-tetrahydro-1H-3-benzazepin-7-yl)-1,2,4-oxadiazol-5-yl]-2-[(2,2,2-trifluoroethyl)oxy]benzonitrile hydrochloride (132 mg) as a pale yellow solid.

LCMS (Method formate): Retention time 0.91 min, MH⁺=472

### Example 192

### 2-[6-(5-{3-chloro-4-[(1-methylethyl)oxy]phenyl}-1,2,4-oxadiazol-3-yl)-5-methyl-3,4-dihydro-2(1H)-isoquinolinyl]-N-[(2S)-2,3-dihydroxypropyl]acetamide hydrochloride

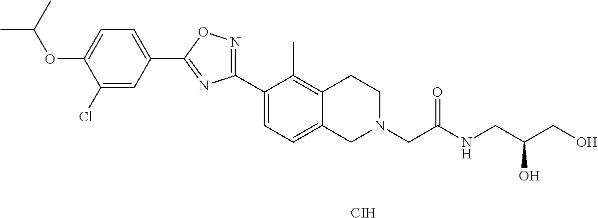

Finely crushed [6-(5-{3-chloro-4-[(1-methylethyl)oxy]phenyl}-1,2,4-oxadiazol-3-yl)-5-methyl-3,4-dihydro-2(1H)-isoquinolinyl]acetic acid (Preparation 10) (88 mg, 0.2 mmol) in a mixture of DMF (2 ml) and NMP (2 ml) was warmed and then cooled to room temperature. DIPEA (0.11 ml, 0.60 mmol) was added, then HATU (76 mg, 0.20 mmol) and after 5 min (2S)-3-amino-1,2-propanediol (27 mg, 0.30 mmol). After 30 min, DIPEA (0.056 ml) was added, then HATU (38 mg) and after 5 min (2S)-3-amino-1,2-propanediol (13 mg) and stirring continued for 15 min. The reaction was partitioned between ethyl acetate and saturated sodium hydrogen carbonate, the aqueous extracted and the combined organic washed with saturated sodium hydrogen carbonate (x3), dried (MgSO₄) and concentrated in vacuo. Purification of the residual solid by MDAP (Method HpH) was attempted, during which process some product crystallised.

The residue from MDAP purification was dissolved in DCM and dried (hydrophobic frit) and concentrated in vacuo. The residue was dissolved in 1,4-dioxane (3 ml) and treated with hydrogen chloride in 1,4-dioxane (4N, 1 ml). The mixture was concentrated in vacuo and the residue dried under vacuum for 12 h to give 2-[6-(5-{3-chloro-4-[(1-methylethyl)oxy]phenyl}-1,2,4-oxadiazol-3-yl)-5-methyl-3,4-dihydro-2(1H)-isoquinolinyl]-N-[(2S)-2,3-dihydroxypropyl]acetamide hydrochloride (15 mg, 14%) as a white solid.

LCMS (Method HpH): Retention time 1.26 min, MH⁺=515/517

The residue which crashed out during the MDAP experiment was triturated with diethyl ether and the solid isolated by filtration. The solid was dissolved in in 1,4-dioxane (3 ml) and treated with hydrogen chloride in 1,4-dioxane (4N, 1 ml). The mixture was concentrated in vacuo and the residue dried under vacuum for 12 h to give 2-[6-(5-{3-chloro-4-[(1-methylethyl)oxy]phenyl}-1,2,4-oxadiazol-3-yl)-5-methyl-3,4-dihydro-2(1H)-isoquinolinyl]-N-[(2S)-2,3-dihydroxypropyl]acetamide hydrochloride (14 mg, 13%) as a white solid.

LCMS (Method HpH): Retention time 1.26 min, MH⁺=515/517

### Example 193

### 2-[6-(5-{3-chloro-4-[(1-methylethyl)oxy]phenyl}-1,2,4-oxadiazol-3-yl)-5-methyl-3,4-dihydro-2(1H)-isoquinolinyl]-N-[2-hydroxy-1-(hydroxymethyl)ethyl]acetamide hydrochloride

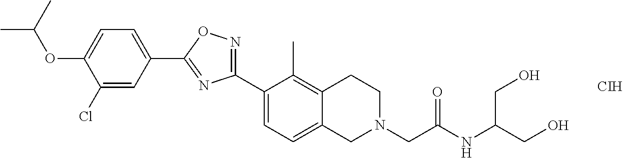

Finely crushed [6-(5-{3-chloro-4-[(1-methylethyl)oxy]phenyl}-1,2,4-oxadiazol-3-yl)-5-methyl-3,4-dihydro-2(1H)-isoquinolinyl]acetic acid (Preparation 10) (88 mg, 0.2 mmol) in a mixture of DMF (2 ml) and NMP (2 ml). The solution was warmed and cooled to room temperature. DIPEA (0.18 ml, 1.0 mmol) was added, then HATU (76 mg, 0.20 mmol) and after 5 min 2-amino-1,3-propanediol (38 mg, 0.30 mmol). After 30 min, further portions of HATU (38 mg) and DIPEA (0.09 ml) were added, followed after 5 min by amine. After 15 min the reaction was partitioned between ethyl acetate and sodium hydrogen carbonate, the aqueous extracted and the combined organic washed with saturated sodium hydrogen carbonate (x3), dried (MgSO₄) and concentrated in vacuo. The residual oil was purified by MDAP (Method HpH). The residue was dissolved in DCM, dried (hydrophobic frit) and concentrated in vacuo. The residue was dissolved in 1,4-dioxane (3 ml), treated with hydrogen chloride in 1,4-dioxane (4N, 1 ml). The mixture was concentrated in vacuo and dried overnight under vacuum, to give 2-[6-(5-{3-chloro-4-[(1-methylethyl)oxy]phenyl}-1,2,4-oxadiazol-3-yl)-5-methyl-3,4-dihydro-2(1H)-isoquinolinyl]-N-[2-hydroxy-1-(hydroxymethyl)ethyl)acetamide hydrochloride (38 mg, 34%) as a white solid.

LCMS (Method HpH): Retention time 1.25 min, MH⁺=515/517

### Example 194

### 2-[7-(5-{3-cyano-4-[(1-methylethyl)oxy]phenyl}-1,2,4-oxadiazol-3-yl)-1,2,4,5-tetrahydro-3H-3-benzazepin-3-yl]-N-[(2S)-2,3-dihydroxypropyl]acetamide hydrochloride

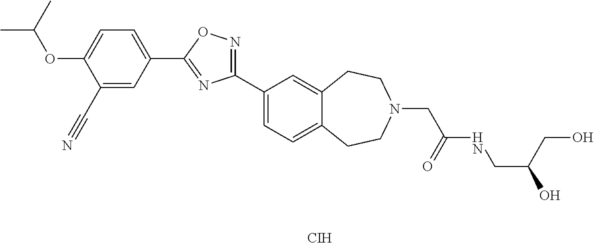

To a solution of [7-(5-{3-cyano-4-[(1-methylethyl)oxy]phenyl}-1,2,4-oxadiazol-3-yl)-1,2,4,5-tetrahydro-3H-3-benzazepin-3-yl]acetic acid (Preparation 89) (86 mg, 0.2 mmol) in DMF (2 ml) at room temperature were added HATU (76 mg, 0.20 mmol) then DIPEA (0.11 ml, 0.6 mmol) and after 5 min (2S)-3-amino-1,2-propanediol (27 mg, 0.3 mmol). The resulting yellow solution was stirred at room temperature overnight. The solvent was removed, the residue partitioned between ethyl acetate and saturated sodium hydrogen carbonate and the aqueous extracted. The combined organic phases were washed with brine, dried (MgSO₄) and concentrated in vacuo. The residue was purified by MDAP (Method HpH). The appropriate fractions were concentrated in vacuo and the residue dissolved in DCM. This solution was dried (hydrophobic frit) and concentrated in vacuo. The residue was dissolved in 1,4-dioxane (3 ml) and treated with hydrogen chloride in 1,4-dioxane (4N). The solvent was removed, the residue triturated with diethyl ether and the solid isolated by filtration to give 2-[7-(5-{3-cyano-4-[(1-methylethyl)oxy]phenyl}-1,2,4-oxadiazol-3-yl)-1,2,4,5-tetrahydro-3H-3-benzazepin-3-yl]-N-[(2S)-2,3-dihydroxypropyl]acetamide hydrochloride (37 mg, 34%) as a white solid.

LCMS (Method HpH): Retention time 1.10 min, MH⁺=506

### Example 195

### 2-[7-(5-{3-cyano-4-[(1-methylethyl)oxy]phenyl}-1,2,4-oxadiazol-3-yl)-1,2,4,5-tetrahydro-3H-3-benzazepin-3-yl]-N-[2-hydroxy-1-(hydroxymethyl)ethyl]acetamide hydrochloride

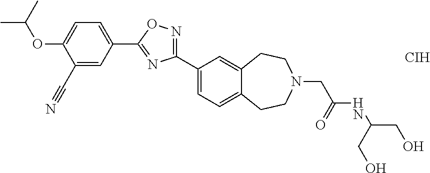

To a solution of [7-(5-{3-cyano-4-[(1-methylethyl)oxy]phenyl}-1,2,4-oxadiazol-3-yl)-1,2,4,5-tetrahydro-3H-3-benzazepin-3-yl]acetic acid (Preparation 89) (86 mg, 0.2 mmol) in DMF (2 ml) at room temperature were added HATU (76 mg, 0.20 mmol) then DIPEA (0.14 ml, 0.8 mmol) and after 5 min 2-amino-1,3-propanediol (38 mg, 0.3 mmol). The resulting yellow solution was stirred at room temperature overnight. The solvent was removed, the residue partitioned between ethyl acetate and saturated sodium hydrogen carbonate and the aqueous extracted. The combined organic phases were washed with brine, dried (MgSO₄) and concentrated in vacuo. The residue was purified by MDAP (Method HpH). The appropriate fractions were concentrated in vacuo and the residue dissolved in DCM. This solution was dried (hydrophobic frit) and concentrated in vacuo. The residue was dissolved in 1,4-dioxane (3 ml) and treated with hydrogen chloride in 1,4-dioxane (4N). The solvent was removed, the residue triturated with diethyl ether and the solid isolated by filtration to give 2-[7-(5-{3-cyano-4-[(1-methylethyl)oxy]phenyl}-1,2,4-oxadiazol-3-yl)-1,2,4,5-tetrahydro-3H-3-benzazepin-3-yl]-N-[2-hydroxy-1-(hydroxymethyl)ethyl]acetamide hydrochloride (41 mg, 38%) as a white solid.

LCMS (Method HpH): Retention time 1.10 min, MH⁺=506

### Example 196

### 5-[3-(3-{N-[(2S)-2,3-dihydroxypropyl]glycyl}-2,3,4,5-tetrahydro-1H-3-benzazepin-7-yl)-1,2,4-oxadiazol-5-yl]-2-[(1-methylethyl)oxy]benzonitrile hydrochloride

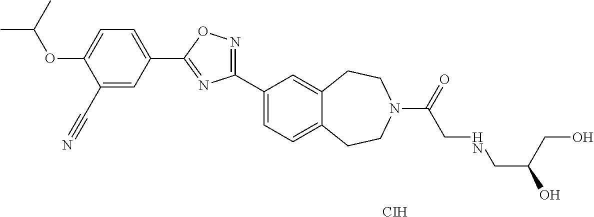

To a solution of 5-{3-[3-(bromoacetyl)-2,3,4,5-tetrahydro-1H-3-benzazepin-7-yl}-1,2,4-oxadiazol-5-yl]-2-[(1-methylethyl)oxy]benzonitrile (Preparation 102) (99 mg, 0.2 mmol) in acetonitrile (3 ml) at room temperature under nitrogen was added potassium carbonate (42 mg, 0.30 mmol) then (2S)-3-amino-1,2-propanediol (91 mg) and the resulting mixture was stirred at 60° C. for 20 min. The reaction was cooled to room temperature, most of the solvent removed and the residue partitioned between ethyl acetate and saturated sodium hydrogen carbonate. The aqueous was extracted, the combined organic phases dried (MgSO₄) and concentrated in vacuo to give a residue which was purified by MDAP (Method HpH). The residue was dissolved in 1,4-dioxane (2 ml) and treated with hydrogen chloride in 1,4-dioxane (4N, 1 ml). The mixture was concentrated in vacuo, the residue triturated with diethyl ether and the solid isolated by filtration to give 5-[3-(3-{N-[(2S)-2,3-dihydroxypropyl]glycyl}-2,3,4,5-tetrahydro-1H-3-benzazepin-7-yl)-1,2,4-oxadiazol-5-yl]-2-[(1-methylethyl)oxy]benzonitrile hydrochloride (21 mg, 19%) as a grey solid.

LCMS (Method HpH): Retention time 1.06 min, MH⁺=506

### Example 197

### 5-[3-(3-{N-[2-hydroxy-1-(hydroxymethyl)ethyl]glycyl}-2,3,4,5-tetrahydro-1H-3-benzazepin-7-yl)-1,2,4-oxadiazol-5-yl]-2-[(1-methylethyl)oxy]benzonitrile hydrochloride

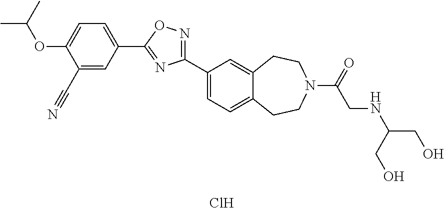

To a solution of 5-{3-[3-(bromoacetyl)-2,3,4,5-tetrahydro-1H-3-benzazepin-7-yl}-1,2,4-oxadiazol-5-yl]-2-[(1-methylethyl)oxy]benzonitrile (Preparation 102) (99 mg, 0.2 mmol) in acetonitrile (3 ml) at room temperature under nitrogen was added potassium carbonate (138 mg, 1.0 mmol) then 2-amino-1,3-propanediol (128 mg, 1.0 mmol) and the resulting mixture was stirred at 60° C. for 20 min. The reaction was cooled to room temperature, filtered and the residue washed with ethyl acetate. The combined filtrate and washings were concentrated, the residue dissolved in ethyl acetate and washed with saturated sodium hydrogen carbonate. The aqueous was extracted with ethyl acetate, the combined organics dried (MgSO₄) and concentrated in vacuo. The residue was purified by MDAP (Method HpH), the residue dissolved in 1,4-dioxane (2 ml) and treated with hydrogen chloride in 1,4-dioxane (4N, 1 ml). The mixture was concentrated in vacuo, the residue triturated with diethyl ether and the solid isolated by filtration to give 5-[3-(3-{N-[2-hydroxy-1-(hydroxymethyl)ethyl)glycyl}-2,3,4,5-tetrahydro-1H-3-benzazepin-7-yl)-1,2,4-oxadiazol-5-yl]-2-[(1-methylethyl)oxy]benzonitrile hydrochloride (20 mg, 18%) as a white solid.

LCMS (Method HpH): Retention time 1.06 min, MH⁺=506

### Example 198

### 5-[3-(2-{N-[(2S)-2-hydroxypropyl]glycyl}-5-methyl-1,2,3,4-tetrahydro-6-isoquinolinyl)-1,2,4-oxadiazol-5-yl]-2-[(1-methylethyl)oxy]benzonitrile hydrochloride

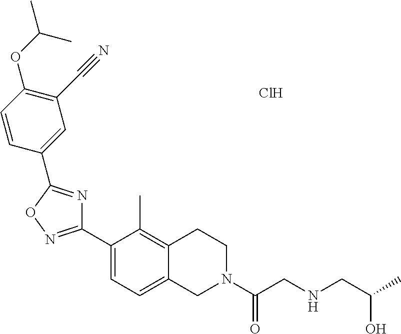

S-1-Amino-2-propanol (76 mg, 1 mmol) was added to a stirred mixture of 5-{3-[2-(bromoacetyl)-5-methyl-1,2,3,4-tetrahydro-6-isoquinolinyl]-1,2,4-oxadiazol-5-yl}-2-[(1-methylethyl)oxy]benzonitrile (Preparation 131) (100 mg, 0.2 mmol) and potassium carbonate (70 mg, 0.5 mmol) in acetonitrile (3 ml). The reaction mixture was stirred at room temperature for 3 h. The reaction mixture was filtered and the solvent evaporated from the filtrate. The residue was chromatographed (methanol/DCM, 5-10%). The product was dissolved in DCM (5 ml) and treated with hydrogen chloride in diethyl ether (1 M, 0.3 ml). The solvent was evaporated and the residue triturated with diethyl ether to give 5-[3-(2-{N-[(2S)-2-hydroxypropyl]glycyl}-5-methyl-1,2,3,4-tetrahydro-6-isoquinolinyl)-1,2,4-oxadiazol-5-yl]-2-[(1-methylethyl)oxy]benzonitrile hydrochloride (22 mg) as an off-white solid.

LCMS (Method formate): Retention time 0.92 min, MH⁺=490

### Example 199

### 5-(5-{3-[2-hydroxy-1-(hydroxymethyl)ethyl]-2,3,4,5-tetrahydro-1H-3-benzazepin-7-yl}-1,3,4-thiadiazol-2-yl)-2-[(1-methylethyl)oxy]benzonitrile hydrochloride

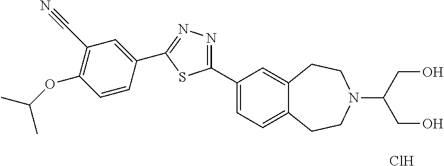

To a solution of 5-{5-[3-(2,2-dimethyl-1,3-dioxan-5-yl)-2,3,4,5-tetrahydro-1H-3-benzazepin-7-yl]-1,3,4-thiadiazol-2-yl}-2-[(1-methylethyl)oxy]benzonitrile (Preparation 190) (249 mg, 0.49 mmol) in THF (2.5 ml was added hydrochloric acid (2M, 2.5 ml). The resulting biphasic mixture was stirred at room temperature for 3 h. The mixture was concentrated under reduced pressure, the residue dissolved in toluene, then concentrated (x2), and the residue subsequently triturated with diethyl ether to give, after filtration 5-(5-{3-[2-hydroxy-1-(hydroxymethyl)ethyl]-2,3,4,5-tetrahydro-1H-3-benzazepin-7-yl}-1,3,4-thiadiazol-2-yl)-2-[(1-methylethyl)oxy]benzonitrile hydrochloride as an off-white solid (125 mg)

LCMS (Method formate): Retention time 0.82 min, MH⁺=465

### Example 200

### 5-(5-{3-[N-(2-hydroxyethyl)glycyl]-2,3,4,5-tetrahydro-1H-3-benzazepin-7-yl}-1,3,4-thiadiazol-2-yl)-2-[(1-methylethyl)oxy]benzonitrile

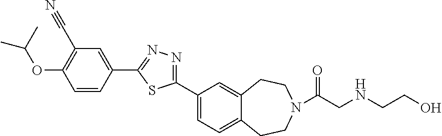

5-{5-[3-(Bromoacetyl)-2,3,4,5-tetrahydro-1H-3-benzazepin-7-yl]-1,3,4-thiadiazol-2-yl}-2-[(1-methylethyl)oxy]benzonitrile (Preparation 191) (128 mg, 0.25 mmol) and potassium carbonate (86 mg, 0.63 mmol) were dissolved in acetonitrile (3 ml) and then 2-aminoethanol (0.076 ml, 1.3 mmol) was added to the mixture. The reaction was stirred at room temperature for 3 h. The solvent was removed under reduced pressure and the residue was partitioned between ethyl acetate and saturated aqueous sodium hydrogen carbonate solution. The aqueous layer was washed with ethyl acetate (30 ml), the combined organic phases were washed with water (30 ml) and the solvent was evaporated in vacuo. The residue was purified by MDAP (Method HpH). The fractions containing product were combined and the solvent was evaporated under reduced pressure. The material obtained was dissolved in DMSO (0.5 ml) and further purified by MDAP on a Xbridge Shield column (19×150 mm i.d., 5 μm packing diameter) eluting with a gradient of 0.1% formic acid in water/0.1% formic acid in methanol at 20 ml/min over 20 min (uv detection: 210-400 nm averaged, MS detection: electrospray, +ve/−ve switching, 100-1000 amu, centroid mode). Evaporation of the solvents from the appropriate fractions under a stream of nitrogen at ambient temperature gave 5-(5-{3-[N-(2-hydroxyethyl)glycyl]-2,3,4,5-tetrahydro-1H-3-benzazepin-7-yl}-1,3,4-thiadiazol-2-yl)-2-[(1-methylethyl)oxy]benzonitrile (3 mg, 2.5%).

LCMS (Method formate): Retention time 0.84 min, MH⁺=492

### Example 201

### 5-(5-{2-[N-(2-hydroxyethyl)glycyl]-5-methyl-1,2,3,4-tetrahydro-6-isoquinolinyl}-1,3,4-thiadiazol-2-yl)-2-[(1-methylethyl)oxy]benzonitrile

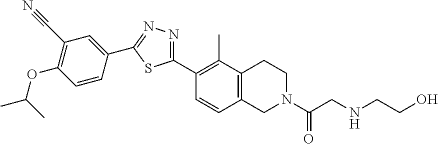

A solution of 2-[(1-methylethyl)oxy]-5-[3-(5-methyl-1,2,3,4-tetrahydro-6-isoquinolinyl)-1,3,4-thiadiazol-2-yl]benzonitrile trifluoroacetate (Preparation 34) (260 mg, 0.52 mmol) and DIPEA (260 μl, 1.5 mmol) in DCM (4 ml) was treated with bromoacetyl bromide (50 μl, 0.58 mmol) and the reaction stirred under nitrogen for 10 min. The reaction mixture was diluted with DCM (6 ml) and washed sequentially with hydrochloric acid (0.5M, 10 ml), saturated aqueous sodium hydrogen carbonate (10 ml) and water (10 ml). The organic was dried (hydrophobic frit) and the solvent evaporated in vacuo. To the resulting foam was added potassium carbonate (180 mg, 1.302 mmol) followed by anhydrous acetonitrile (4 ml). The suspension was treated with ethanolamine (150 μl, 2.5 mmol) and the mixture stirred under nitrogen at room temperature for 15.5 h. The reaction mixture was evaporated under vacuum and the resulting solid partitioned between DCM (10 ml) and water (10 ml). The organic was separated and dried (hydrophobic frit). The solvent was removed under vacuum and the residue dissolved in DCM (5 ml). The solution loaded on a silica cartridge (50 g) and the cartridge eluted with a gradient methanol/DCM (0-70%). The appropriate fractions were combined and the solvent evaporated in vacuo. The residue was further purified by MDAP (Method HpH). The appropriate fractions were combined and the solvent evaporated in vacuo to give 5-(5-{2-[N-(2-hydroxyethyl)glycyl]-5-methyl-1,2,3,4-tetrahydro-6-isoquinolinyl}-1,3,4-thiadiazol-2-yl)-2-[(1-methylethyl)oxy]benzonitrile (59 mg, 23%).

LCMS (Method formate): Retention time 0.86 min, MH⁺=492

### Example 202

### 2-({2-[6-(5-{3-chloro-4-[(1-methylethyl)oxy]phenyl}-1,3,4-thiadiazol-2-yl)-5-methyl-3,4-dihydro-2(1H)-isoquinolinyl]-2-oxoethyl}amino)ethanol

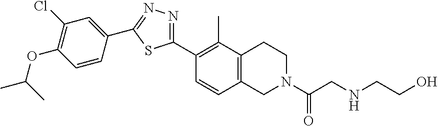

A solution of 6-(5-{3-chloro-4-[(1-methylethyl)oxy]phenyl}-1,3,4-thiadiazol-2-yl)-5-methyl-1,2,3,4-tetrahydroisoquinoline trifluoroacetate (Preparation 192) (58 mg, 0.11 mmol) and DIPEA (58 μl, 0.34 mmol) in DCM (2 ml) was treated with bromoacetyl bromide (11 μl, 0.13 mmol) and stirred under nitrogen for 10 min at room temperature. The reaction mixture was washed sequentially with hydrochloric acid (0.5M, 2 ml), saturated aqueous sodium hydrogen carbonate (2 ml) and water (2 ml). The organic was dried (hydrophobic frit) and the solvent evaporated in vacuo. To the resultant gum was added potassium carbonate (39 mg, 0.282 mmol) followed by acetonitrile (2 ml). The suspension was treated with ethanolamine (34 μl, 0.57 mmol) and the mixture stirred at room temperature for 2.5 h under nitrogen. The reaction mixture was evaporated under vacuum, the solid suspended in water (2 ml) which was then extracted with DCM (3×2 ml). The combined organics were dried (hydrophobic frit) and the solvent was removed under vacuum. The residual gum was purified by MDAP (Method formate). The appropriate fraction was evaporated in vacuo to give 2-({2-[6-(5-{3-chloro-4-[(1-methylethyl)oxy]phenyl}-1,3,4-thiadiazol-2-yl)-5-methyl-3,4-dihydro-2(1H)-isoquinolinyl]-2-oxoethyl}amino)ethanol as a clear gum (18 mg, 32%).

LCMS (Method formate): Retention time 0.96 min, MH⁺=501/503

### Example 203

### 2-[6-(5-{3-chloro-4-[(1-methylethyl)oxy]phenyl}-1,3,4-thiadiazol-2-yl)-5-methyl-3,4-dihydro-2(1H)-isoquinolinyl]-1,3-propanediol hydrochloride

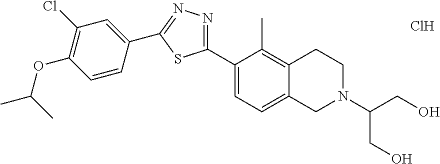

To a solution of 6-(5-{3-chloro-4-[(1-methylethyl)oxy]phenyl}-1,3,4-thiadiazol-2-yl)-2-(2,2-dimethyl-1,3-dioxan-5-yl)-5-methyl-1,2,3,4-tetrahydroisoquinoline (Preparation 194) (130 mg, 0.25 mmol) in THF (1.5 ml) was added hydrochloric acid (2M, 1.5 ml) and the reaction stirred at room temperature for 1.5 h. The resulting mixture was concentrated in vacuo and the crude residue was purified by MDAP (Method HpH). The appropriate fractions were combined and concentrated in vacuo. The residue was treated with hydrogen chloride in diethyl ether (1M, 1 ml) and the precipitate which formed was isolated by filtration, washed with diethyl ether and dried under vacuum to give 2-[6-(5-{3-chloro-4-[(1-methylethyl)oxy]phenyl}-1,3,4-thiadiazol-2-yl)-5-methyl-3,4-dihydro-2(1H)-isoquinolinyl]-1,3-propanediol hydrochloride (71 mg) as a light yellow solid.

LCMS (Method formate): Retention time 0.90 min, MH⁺=474/476

### Example 204

### 5-(5-{5-ethyl-2-[2-hydroxy-1-(hydroxymethyl)ethyl]-1,2,3,4-tetrahydro-6-isoquinolinyl}-1,3-thiazol-2-yl)-2-[(1-methylethyl)oxy]benzonitrile (E204)

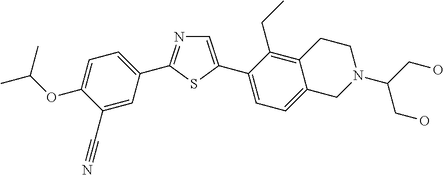

To a solution of 5-[5-(5-ethyl-1,2,3,4-tetrahydro-isoquinolin-6-yl)-thiazol-2-yl]-2-isopropoxybenzonitrile (100 mg, 0.193 mmol) (D208) and Et₃N (0.040 mL, 0.290 mmol) in dichloromethane (10 mL) stirred in air was added 2,2-dimethyl-1,3-dioxan-5-one (37.7 mg, 0.290 mmol) in one portion. The reaction mixture was stirred at rt for 3 hrs. Then sodium triacetoxyborohydride (61.4 mg, 0.290 mmol) was added and the reaction was allowed to stir for additional 4 h. The reaction mixture was diluted with water and extracted with EA, washed with water and brine. After evaporation, the residue was dissolved in THF (4 mL) and water (1 mL). The mixture was stirred at rt for 1 h and evaporated under high-vacuum, dissolved in THF for MDAP to obtain 40 mg 5-(5-{5-ethyl-2-[2-hydroxy-1-(hydroxymethyl)ethyl]-1,2,3,4-tetrahydro-6-isoquinolinyl}-1,3-thiazol-2-yl)-2-[(1-methylethyl)oxy]benzonitrile (yield 35%).

δH (DMSO, 400 MHz): 8.22-8.20 (1H, m), 8.14 (1H, dd), 7.73 (1H, s), 7.36 (1H, d), 7.09-7.07 (1H, m), 6.92-6.91 (1H, m), 4.84-4.82 (1H, m), 4.30-4.29 (2H, m), 3.82-3.81 (2H, m), 3.53-3.49 (3H, m), 2.88-2.87 (2H, m), 2.75-2.74 (2H, m), 2.60-2.56 (2H, m), 1.29 (6H, d), 1.00 (3H, t) ppm. MS (ES⁺): C₂₇H₃₁N₃O₃S requires 477; found; 478 (M+H⁺).

### Example 205

### 5-(5-{5-ethyl-2-[2-hydroxy-1-(hydroxymethyl)ethyl]-1,2,3,4-tetrahydro-6-isoquinolinyl}-1,3,4-thiadiazol-2-yl)-2-[(1-methylethyl)oxy]benzonitrile

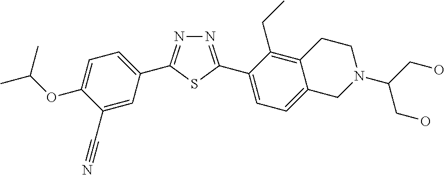

To a solution of 5-ethyl-6-[5-(3-cyano-4-isopropoxy-phenyl)-[1,3,4]thiadiazol-2-yl]-1,2,3,4-tetrahydroisoquinoline (100 mg, 0.195 mmol) (D210) and Et₃N (0.041 mL, 0.293 mmol) in dichloromethane (10 mL) stirred in air was added 2,2-dimethyl-1,3-dioxan-5-one (38.1 mg, 0.293 mmol) in one portion. The reaction mixture was stirred at rt for 3 hrs. Then sodium triacetoxyborohydride (62.0 mg, 0.293 mmol) was added and the reaction was allowed to stir for additional 4 h. The reaction mixture was diluted with water and extracted with EA, washed with water and brine. After evapration, the residue was dissolved in THF (4 mL) and Water (1 mL) and TFA (5 mL) was added. The mixture was stirred at rt for 1 h and evaporated under high-vacuum, dissolved in THF for MDAP to obtain 35 mg 5-(5-{5-ethyl-2-[2-hydroxy-1-(hydroxymethyl)ethyl]-1,2,3,4-tetrahydro-6-isoquinolinyl}-2-pyrimidinyl)-2-[(1-methylethyl)oxy]benzonitrile (yield 36%).

δH (DMSO, 400 MHz): 8.80 (1H, s), 8.62-8.58 (2H, m), 7.42 (1H, d), 7.13-7.12 (1H, m), 5.39-5.37 (1H, m), 4.90-4.84 (1H, m), 4.55-4.51 (2H, m), 3.78-3.77 (4H, m), 3.15-3.06 (4H, m), 2.50-2.49 (2H, m), 1.32 (6H, d), 0.90 (3H, t) ppm. MS (ES⁺): C₂₆H₃₀N₄O₃S requires 478; found; 479 (M+H⁺).

Membrane preparation for S1P1 GTPγS assay

All steps were performed at 4° C. Cells were homogenised within a glass Waring blender for 2 bursts of 15 secs in 200 mls of buffer (50 mM HEPES, 1 mM leupeptin, 25 μg/ml bacitracin, 1 mM EDTA, 1 mM PMSF, 2 μM pepstatin A). The blender was plunged into ice for 5 mins after the first burst and 10-40 mins after the final burst to allow foam to dissipate. The material was then spun at 500 g for 20 mins and the supernatant spun for 36 mins at 48,000 g. The pellet was resuspended in the same buffer as above but without PMSF and pepstatin A. The material was then forced through a 0.6 mm needle, made up to the required volume, (usually x4 the volume of the original cell pellet), aliquoted and stored frozen at −80° C.

### S1P1 GTPγS Assay

S₁P₁ expressing RH7777 membranes (1.5 μg/well) membranes (1.5 μg/well) were homogenised by passing through a 23 G needle. These were then adhered to WGA-coated SPA beads (0.125 mg/well) in assay buffer (HEPES 20 mM, MgCl₂ 10 mM, NaCl 100 mM and pH adjusted to 7.4 using KOH 5M). GDP 10 μM FAC and saponin 90 μg/ml FAC were also added

After 30 minutes precoupling on ice, the bead and membrane suspension was dispensed into white Greiner polypropylene LV 384-well plates (5 μl/well), containing 0.1 l of compound. 5 μl/well [³⁵S]-GTPγS (0.5 nM for S₁P₁ or 0.3 nM for S₁P₃ final radioligand concentration) made in assay buffer was then added to the plates. The final assay cocktail (10.1 μl) was then sealed, spun on a centrifuge, then read immediately on a Viewlux instrument.

Examples 1 to 42 of the invention had a pEC50 >6 in this assay (except for Example 18 with a pEC50 of 5.7 and Example 36 for which no data was recorded).

Alternatively, after 30 minutes precoupling on ice, the bead and membrane suspension was mixed with ³⁵S]-GTPγS (0.5 nM final radioligand concentration) in assay buffer (HEPES 20 mM, MgCl₂ 10 mM, NaCl 100 mM and pH adjusted to 7.4 using KOH 5M) in a 1:1 ratio. The bead, membrane and radioligand suspension was dispensed into white Greiner polypropylene low volume 384-well plates (10 μl/well), containing 0.1 μl of a solution of test compound in 100% DMSO. The final assay cocktail (10.1 μl) was then sealed, spun on a centrifuge, then read immediately on a Viewlux instrument.

Tested in one of the above S1P1 assays all the Examples had a pEC50 of ≧5 except Examples 18, 45, 104 and 129. Examples 13, 14, 16, 17, 28 to 32, 38, 46, 47, 55, 57, 64, 65, 77, 78, 82 to 84, 91, 94, 102, 106, 107, 109, 110, 112 to 117, 132, 143, 145, 149, 150 and 196 had a pEC50 of ≧6. Examples 1 to 12, 15, 19 to 22, 24 to 27, 33 to 35, 39 to 42, 44, 49 to 54, 56, 58 to 63, 66 to 75, 79 to 81, 85, 86, 87, 97, 111, 118, 123 to 125, 133 to 135, 140, 146 to 148, 153, 154, 156, 158, 159, 186, 187, 189, 194, 195, 197 had a pEC50 of ≧7. Examples 43, 48, 76, 87, 90, 92, 93, 95, 96, 98 to 101, 105 and 119 to 122 had a pEC50 of ≧8. Examples 89 and 155 had a pEC50 of 9. No data was taken for Examples 36, 103, 108, 126 to 128, 130, 131, 136 to 139, 141, 142, 144, 152, 157, 160 to 185, 188, 190 to 193 and 198 to 203.

### S1P1 Tango Assay—384 Well Format

Recombinant EDG1-bla/U2OS cells (contain the human Endothelial Differentiation Gene 1 (EDG1) linked to a TEV protease site and a Ga14-VP16 transcription factor stably integrated into the Tango GPCR-bla U20S parental cell line) were harvested from growth medium and passaged into assay medium (Invitrogen Freestyle Expression Medium). The cells were starved for 24 hours at 37° C., 5% CO₂, harvested and resuspended in assay medium at a density of ˜200,000 cells/ml. All test compounds were dissolved in DMSO at a concentration of 10 mM and were prepared in 100% DMSO to provide 10 point dose response curves. Test compounds prepared by Bravo (Velocity11) were added to wells in columns 2-11 and 13-22; DMSO was added to wells in columns 12 and 23 as unstimulated controls and assay medium was added to wells in columns 1 and 24 as cell-free controls. An S1P1 agonist was added to wells in row 2, columns 2-11 as stimulated controls and test compounds were added to wells in row 2, columns 13-22 and rows 3-15, columns 2-11/13-22 (row 1 and 16 were empty and not used). Compounds in solution were added to the assay plate (Greiner 781090) using an Echo (Labcyte) dose-response program (50 nl/well). The unstimulated and cell-free controls were loaded with 50 nl/well pure DMSO to ensure that the DMSO concentration was constant across the plate for all assays.

50 μl of the cell suspension was added to each well in columns 2-23 of the plate (˜10,000 cells per well). 50 μl of assay medium was added to each well in the cell-free controls (columns 1 and 24). The cells were incubated overnight at 37° C./5% CO₂.

10 μl of 6× substrate mixture (LiveBLAzer™-FRET B/G substrate (CCF4-AM) Cat # K1096 from Invitrogen, Inc.) was added to each well using Bravo and the plates incubated at room temperature for 2 h in the dark. The plate was finally read on EnVision using one excitation channel (409 nm) and two emission channels (460 nm and 530 nm).

The blue/green emission ratio (460 nm/530 nm) was calculated for each well, by dividing the background-subtracted Blue emission values by the background-subtracted Green emission values. The dose response curve is based on sigmoidal dose-response model. All ratio data was normalized based upon the maximum emission ratio of positive control and minimum emission ratio of negative control (DMSO) on each plate. The intrinsic activity (IA) of each compound would be the normalized percentage of its maximum response after curve fitting.

Example 204 had a pEC50 >6 in this assay. Example 205 had a pEC50>7 in this assay.

### S1P3 GTPγS Assay

S₁P₃ expressing RBL membranes (1.5 μg/well) were homogenised by passing through a 23 G needle. These were then adhered to WGA-coated SPA beads (0.125 mg/well) in assay buffer (HEPES 20 mM, MgCl₂ 10 mM, NaCl 100 mM and pH adjusted to 7.4 using KOH 5M). GDP 10 μM FAC and saponin 90 μg/ml FAC were also added

After 30 minutes precoupling on ice, the bead and membrane suspension was dispensed into white Greiner polypropylene LV 384-well plates (5 μl/well), containing 0.1 μl of compound. 5 μl/well [³⁵S]-GTPγS (0.5 nM for S₁P₁ or 0.3 nM for S₁P₃ final radioligand concentration) made in assay buffer was then added to the plates. The final assay cocktail (10.1 μl) was then sealed, spun on a centrifuge, then read immediately on a Viewlux instrument.

Examples 1 to 42 had a pEC50 <6 in this assay.

### Alternative Membrane Preparation for S1P3 GTPγS Assay

All steps were performed at 4° C. Cells were homogenised within a glass Waring blender for 2 bursts of 15 secs in 200 mls of buffer (50 mM HEPES, 1 mM leupeptin, 25 μg/ml bacitracin, 1 mM EDTA, 1 mM PMSF, 2 μM pepstatin A). The blender was plunged into ice for 5 mins after the first burst and 10-40 mins after the final burst to allow foam to dissipate. The material was then spun at 500 g for 20 mins and the supernatant spun for 36 mins at 48,000 g. The resultant pellet was resuspended in the same buffer without PMSF and pepstatin A but containing 10% w/v sucrose. The membrane suspension was then layered on top of buffer without PMSF and pepstatin A but containing 40% w/v sucrose and spun at 100,000 g for 60 mins. The cloudy interface between the 2 sucrose layers was removed and resuspended in buffer without PMSF and pepstatin A. The material was spun at 48,000 g for 45 mins.

The resultant cell pellet was resuspended in the required volume in buffer without PMSF and pepstatin A, (usually x4 the volume of the original cell pellet), aliquoted and stored frozen at −80° C.

Alternative S1P3 Purified Membrane GTPγS assay S₁P₃ expressing RBL membranes (0.44 μg/well) purified through a sucrose gradient were homogenised by passing through a 23 G needle. These were then adhered to WGA-coated SPA beads (GE Healthcare 0.5 mg/well) in assay buffer (HEPES 20 mM, MgCl₂ 10 mM, NaCl 100 mM and pH adjusted to 7.4 using KOH 5M). 4 g/well of Saponin was added.

After 30 minutes precoupling on ice, 5 μM GDP final assay concentration was added to the bead and membrane suspension. The bead, membrane, Saponin and GDP suspension was mixed with [³⁵S]-GTPγS (Perkin Elmer, 0.3 nM final radioligand concentration) made in assay buffer (HEPES 20 mM, MgCl₂ 10 mM, NaCl 100 mM and pH adjusted to 7.4 using KOH 5M). The bead, membrane and radioligand suspension was dispensed into white Greiner polypropylene 384-well plates (45 μl/well), containing 0.5 μl of a solution of test compound in 100% DMSO. The final assay cocktail (45.5 μl) was then sealed, spun on a centrifuge, then read on a Viewlux instrument following a 3 hour incubation of plates at room temperature.

Tested in one of the above S1P3 assays Examples 1 to 203 had a pEC50 <6 (Example 142 was not tested).

### S1P3 GeneBlazer Assay

GeneBLAzer EDG3-Ga15-NFAT-bla HEK 293T cells (contain the human Endothelial Differentiation G-protein Coupled Receptor 3 (EDG3) and a beta-lactamase reporter gene under control of a NFAT response element and a promiscuous G Protein, Ga15, stably integrated into the GeneBLAzer Ga15-NFAT-bla HEK 293T cell line) were suspended in assay medium (99% DMEM, 1% Dialyzed FBS, 0.1 mM NEAA, 25 mM HEPES (pH 7.3), 100 U/ml penicillin, 100 μg/ml streptomycin) at a density of 312, 500 cells/ml. Add 100 μl/well of the assay medium to the cell-free control wells (column 12) and 100 μl/well of the cell suspension to the test compound wells (row 2-8, column 1-10), the unstimulated control wells (DMSO) (column 11), and stimulated control wells (S1P) (row 1, column 1-10) in a Corning black-well, clear bottom 96-well plate. Cells were incubated at 37° C., 5% CO2 for 24 h.

Add 25 μl of 5× stock solution of test compounds in assay medium with 0.5% DMSO to the test compound wells, 25 μl of 5× stock solution of agonist (S1P) in assay medium with 0.5% DMSO to the stimulated compound wells, and 25 μl of 5× stock solution of 0.5% DMSO in assay medium to the unstimulated control and cell-free Control wells.

After incubation at 37° C., 5% CO2 for 5 h, 25 μl of 6× substrate mixture (6 μl Solution A (1 mg LiveBLAzer™-FRET B/G Substrate (CCF4-AM) in 912 μl DMSO) plus 60 μl Solution B plus 934 μl Solution C) was added to each well and incubate at room temperature for 2 h in dark. The plate was finally read on EnVision for two emission channels (460 nm and 530 nm).

All test compounds were dissolved in DMSO at a concentration of 10 mM and were prepared in 100% DMSO using a 1 in 5 dilution step to provide 10 point dose response curves. The dilutions were transferred to the assay plates ensuring that the DMSO concentration was constant across the plate for all assays.

Calculate the blue/green emission ratio (460 nm/530 nm) for each well, by dividing the background-subtracted Blue emission values by the background-subtracted green emission values. The dose response curve is based on sigmoidal dose-response model. All ratio data was normalized based upon the maximum emission ratio of positive control (S1P) and minimum emission ratio of negative control (DMSO) on each plate. The intrinsic activity (IA) of each compound would be the normalized percentage of its maximum response after curve fitting.

Example 204 and 205 had a pEC50 <5 in this assay.

### S1P-1 β-Arrestin Recruitment Assay

β-Arrestin recruitment assays were carried out using the PathHunter CHO-K1 EDG1

β-Arrestin cell line (DiscoveRx Corporation) in a chemi-luminescence detection assay. This cell line stably expresses β-Arrestin 2 and S1P1 fused to complementing portions of β-galactosidase (‘EA’ and ‘pro-link’, respectively) which associate upon Arrestin recruitment to form functional β-galactosidase enzyme.

Cells were grown to 80% confluency in Growth Medium (F12 nutrient HAMS supplemented with 10% heat-inactivated USA FBS, 1% L-glutamax, 800 μg/ml Geneticin and 300 μg/ml Hygromycin). Cells were harvested from the flask using Enzyme Free Cell Dissociation Buffer (Gibco) and washed from flasks with Optimem solution (Gibco). Cells were then centrifuged at 1000 rpm for 2-3 min and resuspended in Assay Buffer (Prepared from Sigma kit H1387 supplemented with 20 ml/L HEPES, 4.7 ml/L NaHCO₃, 0.1% pluronic acid F-68 solution, 0.1% BSA and adjusted to pH 7.4 using sodium hydroxide at 1×10⁶ cells/ml. Cells were dispensed into assay plates containing compounds (100n1/well of a solution of test compound in 100% DMSO) at 1×10⁴ cells/well and incubated at 37° C./5% CO2 for 90 min followed by 15 min at room temperature. 5 μl detection mix (1 part Galacton Star, 5 parts Emerald II, 19 parts Assay Buffer; DiscoveRx) were added per well and the plates incubated at room temperature for 60 min. Luminescence was quantified using a Viewlux plate reader.

All Examples had a pEC50 of except Examples 57, 94, 104 and 182. No data was recorded for Examples 142, 200 and 202. Examples 2, 9, 11 to 21, 23, 27 to 29, 32 to 35, 37, 47, 49 to 53, 56, 58 to 63, 67 to 68, 70, 71, 74, 77, 78, 81, 82, 84 to 86, 88, 90, 92, 95 to 99, 101 to 103, 105, 106, 108, 112, 114, 116, 132, 133, 135, 137 to 140, 144, 153, 154, 161, 162, 166 to 169, 174 to 176, 183 to 185, 190, 196 and 203 had a pEC50 of ≧7. Examples 1, 3 to 7, 8, 10, 22, 24 to 26, 39, 40 to 44, 48, 54, 64 to 66, 69, 72, 73, 75, 76, 79, 80, 87, 89, 93, 100, 111, 118, 119, 122 to 131, 134, 136, 146 to 148, 155 to 159, 164, 165, 170, 178 to 180, 186, 187, 189, 191 to 195, 197 and 200 had a pEC50 of ≧8.

1. A compound of formula (I) or a pharmaceutically acceptable salt thereof: 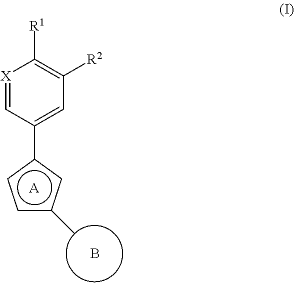
X is CH or N;
R¹ is OR³, NHR⁴, R⁵, NR⁶R⁷, R⁸ or optionally fluorinated C₍₃₋₆₎cycloalkyl;
R² is hydrogen, halogen, cyano, trifluoromethyl, C₍₁₋₂₎alkoxy and C₍₁₋₃₎alkyl optionally substituted by halogen;
R³ and R⁴ are C₍₁₋₅₎alkyl optionally interrupted by O and optionally substituted by F or (CH₂)₍₀₋₁₎C₍₃₋₅₎cycloalkyl optionally substituted by F;
R⁵ is C₍₁₋₆₎alkyl optionally substituted by F;
R⁶ and R⁷ are independently selected from C₍₁₋₅₎alkyl optionally interrupted by O and optionally substituted by F and optionally fluorinated C₍₃₋₅₎cycloalkyl with the proviso that the combined number of carbon atoms in R⁶ and R⁷ does not exceed 6;
R⁸ is a 3 to 6 membered, nitrogen-containing heterocyclyl ring optionally substituted by F selected from aziridinyl, azetidinyl, pyrrolidinyl, piperidinyl and morpholinyl, all attached via the nitrogen atom;
A is a 5-membered heterocyclic ring selected from the following: 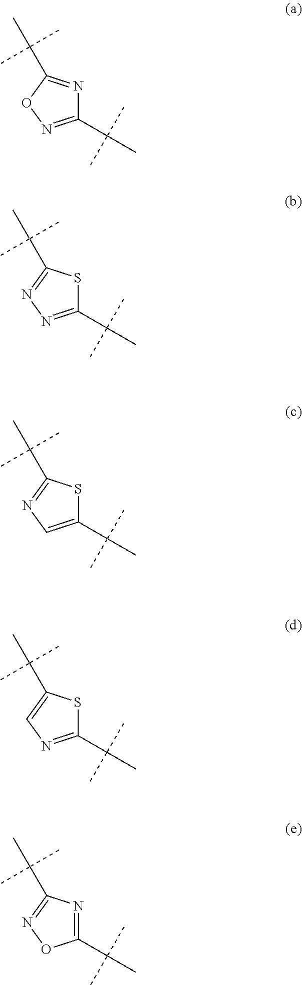
B is a bicyclic ring selected from the following: 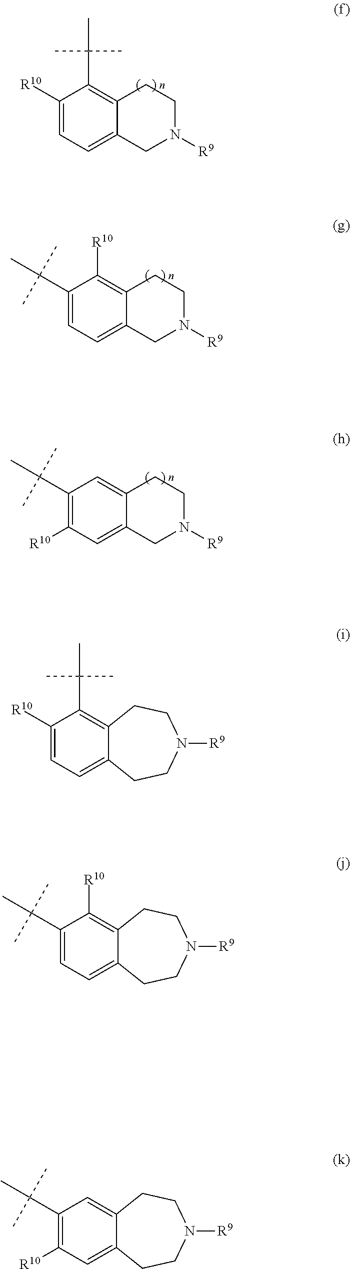
R⁹ is C₍₁₋₄₎alkyl substituted by at least one OH and optionally interrupted by O, C₍₁₋₄₎alkyl interrupted by O, C₍₂₋₄₎alkyl substituted by SO₂C₍₁₋₄₎alkyl, C₍₁₋₄₎alkylCONR¹¹R¹², C₍₂₋₄₎alkyl NR¹³CONR¹¹R¹², C₍₂₋₄₎alkylNR¹³COOR¹², C₍₂₋₄₎alkylOCONR¹¹R¹², C₍₂₋₄₎alkyl NR¹³COR¹² or COC₍₁₋₄₎alkylNR¹¹R¹²;
when R⁹ is COC₍₁₋₄₎alkylNR¹¹R¹² the alkyl chain may be optionally substituted by C₍₁₋₃₎alkylOH or interrupted by O;
when R⁹ is C₍₁₋₄₎alkylCONR¹¹R¹², C₍₂₋₄₎alkylNR¹³COOR¹², C₍₂₋₄₎alkylNR¹³CONR¹¹R¹², C₍₂₋₄₎alkylNR¹³COR¹² and comprises an alkyl chain of at least two carbon atoms at the point of attachment to the B ring it may be optionally substituted by halogen, OC₍₁₋₃₎alkyl or OH;
R¹⁰ is hydrogen or C₍₁₋₃₎alkyl optionally substituted by halogen;
R¹¹, R¹² and R¹³ are independently selected from hydrogen or C₍₁₋₃₎alkyl optionally substituted by F or hydroxyl and optionally interrupted by O;
R¹¹ and R¹² together with the nitrogen atom to which they are attached may be linked to form a 4-6 membered heterocyclyl ring, wherein the 4- to 6-membered heterocyclyl ring optionally contains an oxygen atom and is optionally substituted by one or two substituents independently selected from F and OH;
R¹² and R¹³, together with the atoms to which they are attached may be linked to form an optionally unsaturated 5-7 membered heterocyclyl ring, wherein the 5- to 7-membered heterocyclyl ring optionally contains an oxygen atom and is optionally substituted by one or two substituents independently selected from F and OH; and
n is 0, 1 or 2.

2. A compound of formula (I) or a pharmaceutically acceptable salt thereof: 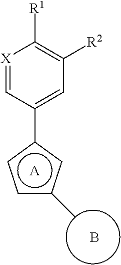
wherein
X is CH or N;
R¹ is OR³;
R³ is isopropyl;
R² is chloro or cyano;
A is 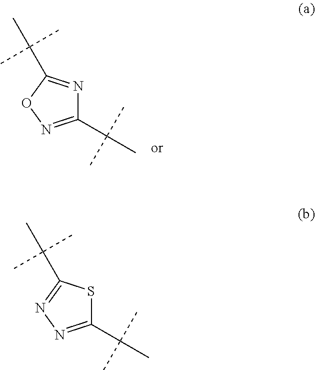
B is (f), (g), (i) or (j) 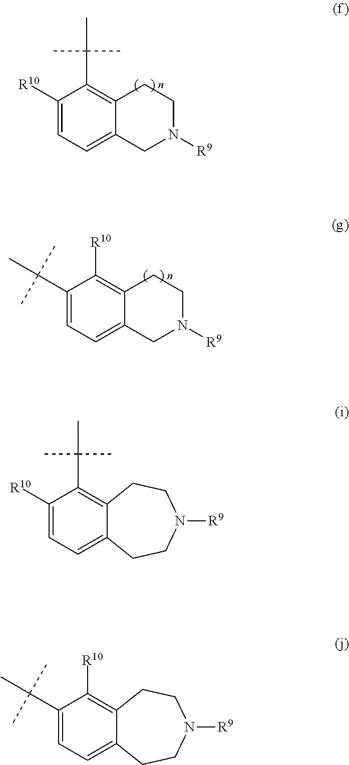
R⁹ is C₍₁₋₄₎alkyl substituted by at least one OH and optionally interrupted by O, C₍₁₋₄₎alkyl interrupted by O or C₍₂₋₄₎alkyl substituted by SO₂C₍₁₋₃₎alkyl;
R¹⁰ is hydrogen or methyl; and
n is 1.

3. A compound according to claim 1 selected from the group consisting of:
3-[6-(5-{3-Cyano-4-[(1-methylethyl)oxy]phenyl}-1,2,4-oxadiazol-3-yl)-5-methyl-3,4-dihydro-2(1H)-isoquinolinyl]propanamide;
3-[6-(5-{3-Cyano-4-[(1-methylethyl)oxy]phenyl}-1,2,4-oxadiazol-3-yl)-5-methyl-3,4-dihydro-2(1H)-isoquinolinyl]-N,N-dimethylpropanamide;
2-[6-(5-{3-Cyano-4-[(1-methylethyl)oxy]phenyl}-1,2,4-oxadiazol-3-yl)-5-methyl-3,4-dihydro-2(1H)-isoquinolinyl]acetamide;
4-[6-(5-{3-Cyano-4-[(1-methylethyl)oxy]phenyl}-1,2,4-oxadiazol-3-yl)-5-methyl-3,4-dihydro-2(1H)-isoquinolinyl]butanamide;
4-[6-(5-{3-Cyano-4-[(1-methylethyl)oxy]phenyl}-1,2,4-oxadiazol-3-yl)-5-methyl-3,4-dihydro-2(1H)-isoquinolinyl]-N-methylbutanamide;
5-{3-[2-(2,3-Dihydroxypropyl)-5-methyl-1,2,3,4-tetrahydro-6-isoquinolinyl]-1,2,4-oxadiazol-5-yl}-2-[(1-methylethyl)oxy]benzonitrile;
5-{3-[2-(2-Hydroxyethyl)-5-methyl-1,2,3,4-tetrahydro-6-isoquinolinyl]-1,2,4-oxadiazol-5-yl}-2-[(1-methylethyl)oxy]benzonitrile;
5-{3-[2-(3-Hydroxypropyl)-5-methyl-1,2,3,4-tetrahydro-6-isoquinolinyl]-1,2,4-oxadiazol-5-yl}-2-[(1-methylethyl)oxy]benzonitrile;
2-[(1-Methylethyl)oxy]-5-(3-{5-methyl-2-[2-(methyloxy)ethyl]-1,2,3,4-tetrahydro-6-isoquinolinyl}-1,2,4-oxadiazol-5-yl)benzonitrile;
3-[6-(5-{3-Cyano-4-[(1-methylethyl)oxy]phenyl}-1,3,4-thiadiazol-2-yl)-5-methyl-3,4-dihydro-2(1H)-isoquinolinyl]propanamide;
3-[6-(5-{3-Cyano-4-[(1-methylethyl)oxy]phenyl}-1,3,4-thiadiazol-2-yl)-5-methyl-3,4-dihydro-2(1H)-isoquinolinyl]-N-methylpropanamide;
3-[6-(5-{3-Cyano-4-[(1-methylethyl)oxy]phenyl}-1,3,4-thiadiazol-2-yl)-5-methyl-3,4-dihydro-2(1H)isoquinolinyl]-N,N-dimethylpropanamide;
4-[6-(5-{3-Cyano-4-[(1-methylethyl)oxy]phenyl}-1,3,4-thiadiazol-2-yl)-5-methyl-3,4-dihydro-2(1H)-isoquinolinyl]butanamide;
4-[6-(5-{3-Cyano-4-[(1-methylethyl)oxy]phenyl}-1,3,4-thiadiazol-2-yl)-5-methyl-3,4-dihydro-2(1H)-isoquinolinyl]-N-methylbutanamide;
5-{5-[2-(2-Hydroxyethyl)-5-methyl-1,2,3,4-tetrahydro-6-isoquinolinyl]-1,3,4-thiadiazol-2-yl}-2-[(1-methylethyl)oxy]benzonitrile;
5-{5-[2-(3-Hydroxypropyl)-5-methyl-1,2,3,4-tetrahydro-6-isoquinolinyl]-1,3,4-thiadiazol-2-yl}-2-[(1-methylethyl)oxy]benzonitrile;
2-[(1-Methylethyl)oxy]-5-(5-{5-methyl-2-[2-(methyloxy)ethyl]-1,2,3,4-tetrahydro-6-isoquinolinyl}-1,3,4-thiadiazol-2-yl)benzonitrile;
5-{5-[2-(2,3-Dihydroxypropyl)-5-methyl-1,2,3,4-tetrahydro-6-isoquinolinyl]-1,3,4-thiadiazol-2-yl}-2-[(1-methylethyl)oxy]benzonitrile;
5-{3-[3-(2-Hydroxyethyl)-2,3,4,5-tetrahydro-1H-3-benzazepin-7-yl]-1,2,4-oxadiazol-5-yl}-2-[(1-methylethyl)oxy]benzonitrile;
5-{3-[3-(3-Hydroxypropyl)-2,3,4,5-tetrahydro-1H-3-benzazepin-7-yl]-1,2,4-oxadiazol-5-yl}-2-[(1-methylethyl)oxy]benzonitrile;
2-[(1-Methylethyl)oxy]-5-(3-{3-[2-(methyloxy)ethyl]-2,3,4,5-tetrahydro-1H-3-benzazepin-7-yl}-1,2,4-oxadiazol-5-yl)benzonitrile;
3-[7-(5-{3-Cyano-4-[(1-methylethyl)oxy]phenyl}-1,2,4-oxadiazol-3-yl)-1,2,4,5-tetrahydro-3H-3-benzazepin-3-yl]propanamide;
3-[7-(5-{3-Cyano-4-[(1-methylethyl)oxy]phenyl}-1,2,4-oxadiazol-3-yl)-1,2,4,5-tetrahydro-3H-3-benzazepin-3-yl]-N,N-dimethylpropanamide;
4-[7-(5-{3-Cyano-4-[(1-methylethyl)oxy]phenyl}-1,2,4-oxadiazol-3-yl)-1,2,4,5-tetrahydro-3H-3-benzazepin-3-yl]-N-methylbutanamide;
4-[7-(5-{3-Cyano-4-[(1-methylethyl)oxy]phenyl}-1,2,4-oxadiazol-3-yl)-1,2,4,5-tetrahydro-3H-3-benzazepin-3-yl]butanamide;
4-[7-(5-{3-Cyano-4-[(1-methylethyl)oxy]phenyl}-1,2,4-oxadiazol-3-yl)-1,2,4,5-tetrahydro-3H-3-benzazepin-3-yl]-N,N-dimethylbutanamide;
5-{3-[3-(2,3-Dihydroxypropyl)-2,3,4,5-tetrahydro-1H-3-benzazepin-7-yl]-1,2,4-oxadiazol-5-yl}-2-[(1-methylethyl)oxy]benzonitrile;
5-{5-[3-(2-Hydroxyethyl)-2,3,4,5-tetrahydro-1H-3-benzazepin-7-yl]-1,3,4-thiadiazol-2-yl}-2-[(1-methylethyl)oxy]benzonitrile;
5-{5-[3-(3-Hydroxypropyl)-2,3,4,5-tetrahydro-1H-3-benzazepin-7-yl]-1,3,4-thiadiazol-2-yl}-2-[(1-methylethyl)oxy]benzonitrile;
2-[(1-Methylethyl)oxy]-5-(5-{3-[2-(methyloxy)ethyl]-2,3,4,5-tetrahydro-1H-3-benzazepin-7-yl}-1,3,4-thiadiazol-2-yl)benzonitrile;
2-[6-(5-{3-Cyano-4-[(1-methylethyl)oxy]phenyl}-1,2,4-oxadiazol-3-yl)-1,2,4,5-tetrahydro-3H-3-benzazepin-3-yl]acetamide;
3-[6-(5-{3-Cyano-4-[(1-methylethyl)oxy]phenyl}-1,2,4-oxadiazol-3-yl)-1,2,4,5-tetrahydro-3H-3-benzazepin-3-yl]propanamide;
5-{3-[3-(3-Hydroxypropyl)-2,3,4,5-tetrahydro-1H-3-benzazepin-6-yl]-1,2,4-oxadiazol-5-yl}-2-[(1-methylethyl)oxy]benzonitrile;
5-{3-[3-(2-Hydroxyethyl)-2,3,4,5-tetrahydro-1H-3-benzazepin-6-yl]-1,2,4-oxadiazol-5-yl}-2-[(1-methylethyl)oxy]benzonitrile;
5-{3-[3-(2,3-Dihydroxypropyl)-2,3,4,5-tetrahydro-1H-3-benzazepin-6-yl]-1,2,4-oxadiazol-5-yl}-2-[(1-methylethyl)oxy]benzonitrile;
5-{5-[2-(3-Hydroxypropyl)-1,2,3,4-tetrahydro-5-isoquinolinyl]-1,3,4-thiadiazol-2-yl}-2-[(1-methylethyl)oxy]benzonitrile;
2-[(1-Methylethyl)oxy]-5-(5-{2-[2-(methyloxy)ethyl]-1,2,3,4-tetrahydro-5-isoquinolinyl}-1,3,4-thiadiazol-2-yl)benzonitrile;
5-{5-[2-(2-hydroxyethyl)-1,2,3,4-tetrahydro-5-isoquinolinyl]-1,3,4-thiadiazol-2-yl}-2-[(1-methylethyl)oxy]benzonitrile;
5-[3-(2-β-Alanyl-5-methyl-1,2,3,4-tetrahydro-6-isoquinolinyl)-1,2,4-oxadiazol-5-yl]-2-[(1-methylethyl)oxy]benzonitrile;
2-[(1-Methylethyl)oxy]-5-{3-[5-methyl-2-(N-methyl-alanyl)-1,2,3,4-tetrahydro-6-isoquinolinyl]-1,2,4-oxadiazol-5-yl}benzonitrile;
5-{3-[2-(4-Aminobutanoyl)-5-methyl-1,2,3,4-tetrahydro-6-isoquinolinyl]-1,2,4-oxadiazol-5-yl}-2-[(1-methylethyl)oxy]benzonitrile;
5-[3-(2-Glycyl-5-methyl-1,2,3,4-tetrahydro-6-isoquinolinyl)-1,2,4-oxadiazol-5-yl]-2-[(1-methylethyl)oxy]benzonitrile;
3-[6-(5-{3-cyano-4-[(1-methylethyl)oxy]phenyl}-1,2,4-oxadiazol-3-yl)-5-methyl-3,4-dihydro-2(1H)-isoquinolinyl]-N-methylpropanamide;
4-[7-(5-{3-cyano-4-[(1-methylethyl)oxy]phenyl}-1,2,4-oxadiazol-3-yl)-1,2,4,5-tetrahydro-3H-3-benzazepin-3-yl]-N-ethylbutanamide;
3-[5-(5-{3-cyano-4-[(1-methylethyl)oxy]phenyl}-1,3,4-thiadiazol-2-yl)-3,4-dihydro-2(1H)-isoquinolinyl]-N-methylpropanamide;
3-[5-(5-{3-cyano-4-[(1-methylethyl)oxy]phenyl}-1,3,4-thiadiazol-2-yl)-3,4-dihydro-2(1H)-isoquinolinyl]-N,N-dimethylpropanamide;
3-[6-(5-{3-cyano-4-[(1-methylethyl)oxy]phenyl}-1,3,4-thiadiazol-2-yl)-3,4-dihydro-2(1H)-isoquinolinyl]propanamide;
5-[3-(3-glycyl-2,3,4,5-tetrahydro-1H-3-benzazepin-7-yl)-1,2,4-oxadiazol-5-yl]-2-[(1-methylethyl)oxy]benzonitrile trifluoroacetate;
5-{3-[3-(2,3-dihydroxypropyl)-2,3,4,5-tetrahydro-1H-3-benzazepin-7-yl]-1,2,4-oxadiazol-5-yl}-2-[(1-methylethyl)amino]benzonitrile;
5-{3-[3-(2,3-dihydroxypropyl)-2,3,4,5-tetrahydro-1H-3-benzazepin-7-yl]-1,2,4-oxadiazol-5-yl}-2-(propylamino)benzonitrile;
5-{3-[3-(2,3-dihydroxypropyl)-2,3,4,5-tetrahydro-1H-3-benzazepin-7-yl]-1,2,4-oxadiazol-5-yl}-2-(propyloxy)benzonitrile;
5-[3-(3-glycyl-2,3,4,5-tetrahydro-1H-3-benzazepin-7-yl)-1,2,4-oxadiazol-5-yl]-2-[(1-methylethyl)amino]benzonitrile;
5-[3-(3-glycyl-2,3,4,5-tetrahydro-1H-3-benzazepin-7-yl)-1,2,4-oxadiazol-5-yl]-2-(propylamino)benzonitrile;
2-{[2-fluoro-1-(fluoromethyl)ethyl)oxy}-5-[3-(3-glycyl-2,3,4,5-tetrahydro-1H-3-benzazepin-7-yl)-1,2,4-oxadiazol-5-yl]benzonitrile;
5-{3-[3-(2,3-dihydroxypropyl)-2,3,4,5-tetrahydro-1H-3-benzazepin-7-yl]-1,2,4-oxadiazol-5-yl}-2-(3-fluoro-1-pyrrolidinyl)benzonitrile;
5-[3-(2-D-allothreonyl-5-methyl-1,2,3,4-tetrahydro-6-isoquinolinyl)-1,2,4-oxadiazol-5-yl]-2-[(1-methylethyl)oxy]benzonitrile;
2-(3-fluoro-1-pyrrolidinyl)-5-[3-(3-glycyl-2,3,4,5-tetrahydro-1H-3-benzazepin-7-yl)-1,2,4-oxadiazol-5-yl]benzonitrile;
5-[3-(3-D-allothreonyl-2,3,4,5-tetrahydro-1H-3-benzazepin-7-yl)-1,2,4-oxadiazol-5-yl]-2-[(1-methylethyl)oxy]benzonitrile;
2-[(1-methylethyl)oxy]-5-[3-(3-L-threonyl-2,3,4,5-tetrahydro-1H-3-benzazepin-7-yl)-1,2,4-oxadiazol-5-yl]benzonitrile;
5-{3-[3-(2,3-dihydroxypropyl)-2,3,4,5-tetrahydro-1H-3-benzazepin-7-yl]-1,2,4-oxadiazol-5-yl}-2-{[2-fluoro-1-(fluoromethyl)ethyl)oxy}benzonitrile;
2-(ethyloxy)-5-[3-(3-glycyl-2,3,4,5-tetrahydro-1H-3-benzazepin-7-yl)-1,2,4-oxadiazol-5-yl]benzonitrile;
5-[3-(3-glycyl-2,3,4,5-tetrahydro-1H-3-benzazepin-7-yl)-1,2,4-oxadiazol-5-yl]-2-(propyloxy)benzonitrile;
5-{3-[3-(4-aminobutanoyl)-2,3,4,5-tetrahydro-1H-3-benzazepin-7-yl]-1,2,4-oxadiazol-5-yl}-2-[(1-methylethyl)oxy]benzonitrile;
5-(3-{2-[2-hydroxy-1-(hydroxymethyl)ethyl]-5-methyl-1,2,3,4-tetrahydro-6-isoquinolinyl}-1,2,4-oxadiazol-5-yl)-2-[(1-methylethyl)oxy]benzonitrile;
5-{3-[3-(N-methyl-b-alanyl)-2,3,4,5-tetrahydro-1H-3-benzazepin-7-yl]-1,2,4-oxadiazol-5-yl}-2-[(1-methylethyl)oxy]benzonitrile;
5-(3-{3-[2-hydroxy-1-(hydroxymethyl)ethyl]-2,3,4,5-tetrahydro-1H-3-benzazepin-7-yl}-1,2,4-oxadiazol-5-yl)-2-[(1-methylethyl)oxy]benzonitrile;
5-[3-(3-β-alanyl-2,3,4,5-tetrahydro-1H-3-benzazepin-7-yl)-1,2,4-oxadiazol-5-yl]-2-[(1-methylethyl)oxy]benzonitrile;
5-{3-[3-(2,3-dihydroxypropyl)-2,3,4,5-tetrahydro-1H-3-benzazepin-7-yl]-1,2,4-oxadiazol-5-yl}-2-(ethyloxy)benzonitrile;
5-(3-{2-[(2S)-2,3-dihydroxypropyl]-5-methyl-1,2,3,4-tetrahydro-6-isoquinolinyl}-1,2,4-oxadiazol-5-yl)-2-[(1-methylethyl)oxy]benzonitrile;
5-(3-{3-[(2S)-2,3-dihydroxypropyl]-2,3,4,5-tetrahydro-1H-3-benzazepin-7-yl}-1,2,4-oxadiazol-5-yl)-2-[(1-methylethyl)oxy]benzonitrile;
5-(3-{2-[(2R)-2,3-dihydroxypropyl]-5-methyl-1,2,3,4-tetrahydro-6-isoquinolinyl}-1,2,4-oxadiazol-5-yl)-2-[(1-methylethyl)oxy]benzonitrile;
5-(3-{3-[(2R)-2,3-dihydroxypropyl]-2,3,4,5-tetrahydro-1H-3-benzazepin-7-yl}-1,2,4-oxadiazol-5-yl)-2-[(1-methylethyl)oxy]benzonitrile;
2-[7-(5-{3-cyano-4-[(1-methylethyl)oxy]phenyl}-1,2,4-oxadiazol-3-yl)-1,2,4,5-tetrahydro-3H-3-benzazepin-3-yl]-N-[(2S)-2-hydroxypropyl]acetamide;
5-{3-[2-(2,3-dihydroxypropyl)-1,2,3,4-tetrahydro-5-isoquinolinyl]-1,2,4-oxadiazol-5-yl}-2-[(1-methylethyl)oxy]benzonitrile;
2-[(1-methylethyl)oxy]-5-[3-(3-L-threonyl-2,3,4,5-tetrahydro-1H-3-benzazepin-7-yl)-1,2,4-oxadiazol-5-yl]-3-pyridinecarbonitrile;
2-[7-(5-{3-cyano-4-[(1-methylethyl)oxy]phenyl}-1,2,4-oxadiazol-3-yl)-1,2,4,5-tetrahydro-3H-3-benzazepin-3-yl]-N-(1R)-2-hydroxy-1-methylethyl]acetamide;
2-[(1-methylethyl)oxy]-5-[3-(5-methyl-2-L-threonyl-1,2,3,4-tetrahydro-6-isoquinolinyl)-1,2,4-oxadiazol-5-yl]-3-pyridinecarbonitrile;
2-[7-(5-{3-cyano-4-[(1-methylethyl)oxy]phenyl}-1,2,4-oxadiazol-3-yl)-1,2,4,5-tetrahydro-3H-3-benzazepin-3-yl]-N-[(1S)-2-hydroxy-1-methylethyl]acetamide;
2-[(1-methylethyl)oxy]-5-{3-[3-(2-methyl-L-seryl)-2,3,4,5-tetrahydro-1H-3-benzazepin-7-yl]-1,2,4-oxadiazol-5-yl}benzonitrile;
2-[(1-methylethyl)oxy]-5-{3-[5-methyl-2-(2-methyl-L-seryl)-1,2,3,4-tetrahydro-6-isoquinolinyl]-1,2,4-oxadiazol-5-yl}benzonitrile;
5-(3-{3-[4-(methylamino)butanoyl]-2,3,4,5-tetrahydro-1H-3-benzazepin-7-yl}-1,2,4-oxadiazol-5-yl)-2-[(1-methylethyl)oxy]benzonitrile;
2-[(1-methylethyl)oxy]-5-{3-[3-(2-methyl-D-seryl)-2,3,4,5-tetrahydro-1H-3-benzazepin-7-yl]-1,2,4-oxadiazol-5-yl}benzonitrile;
2-[(1-methylethyl)oxy]-5-{3-[5-methyl-2-(2-methyl-D-seryl)-1,2,3,4-tetrahydro-6-isoquinolinyl]-1,2,4-oxadiazol-5-yl}benzonitrile;
5-(3-{3-[N-(2-hydroxyethyl)glycyl]-2,3,4,5-tetrahydro-1H-3-benzazepin-7-yl}-1,2,4-oxadiazol-5-yl)-2-[(1-methylethyl)oxy]benzonitrile;
5-[3-(2-glycyl-1,2,3,4-tetrahydro-5-isoquinolinyl)-1,2,4-oxadiazol-5-yl]-2-[(1-methylethyl)oxy]benzonitrile;
5-[3-(2-β-alanyl-1,2,3,4-tetrahydro-5-isoquinolinyl)-1,2,4-oxadiazol-5-yl]-2-[(1-methylethyl)oxy]benzonitrile;
2-[(1-methylethyl)oxy]-5-[3-(2-L-seryl-1,2,3,4-tetrahydro-5-isoquinolinyl)-1,2,4-oxadiazol-5-yl]benzonitrile;
2-[(1-methylethyl)oxy]-5-[3-(2-D-seryl-1,2,3,4-tetrahydro-5-isoquinolinyl)-1,2,4-oxadiazol-5-yl]benzonitrile;
2-[(1-methylethyl)oxy]-5-{3-[3-(4-morpholinylacetyl)-2,3,4,5-tetrahydro-1H-3-benzazepin-7-yl]-1,2,4-oxadiazol-5-yl}benzonitrile;
2-[(1-methylethyl)oxy]-5-(3-{3-[2-(4-morpholinyl)-2-oxoethyl]-2,3,4,5-tetrahydro-1H-3-benzazepin-7-yl}-1,2,4-oxadiazol-5-yl)benzonitrile;
5-(3-{3-[2-(3-hydroxy-1-azetidinyl)-2-oxoethyl]-2,3,4,5-tetrahydro-1H-3-benzazepin-7-yl}-1,2,4-oxadiazol-5-yl)-2-[(1-methylethyl)oxy]benzonitrile;
2-(3-fluoro-1-azetidinyl)-5-[3-(3-glycyl-2,3,4,5-tetrahydro-1H-3-benzazepin-7-yl)-1,2,4-oxadiazol-5-yl]benzonitrile;
5-(3-{3-[2-hydroxy-1-(hydroxymethyl)ethyl]-2,3,4,5-tetrahydro-1H-3-benzazepin-7-yl}-1,2,4-oxadiazol-5-yl)-2-[(1-methylethyl)oxy]-3-pyridinecarbonitrile;
5-(3-{3-[(2S)-2,3-dihydroxypropyl]-2,3,4,5-tetrahydro-1H-3-benzazepin-7-yl}-1,2,4-oxadiazol-5-yl)-2-[(1-methylethyl)oxy]-3-pyridinecarbonitrile;
5-(3-{3-[(2R)-2,3-dihydroxypropyl]-2,3,4,5-tetrahydro-1H-3-benzazepin-7-yl}-1,2,4-oxadiazol-5-yl)-2-[(1-methylethyl)oxy]-3-pyridinecarbonitrile;
5-[3-(3-{N-(1R)-2-hydroxy-1-methylethyl]glycyl}-2,3,4,5-tetrahydro-1H-3-benzazepin-7-yl)-1,2,4-oxadiazol-5-yl]-2-[(1-methylethyl)oxy]benzonitrile;
5-[3-(3-{N-[(1S)-2-hydroxy-1-methylethyl]glycyl}-2,3,4,5-tetrahydro-1H-3-benzazepin-7-yl)-1,2,4-oxadiazol-5-yl]-2-[(1-methylethyl)oxy]benzonitrile;
methyl {2-[7-(5-{3-cyano-4-[(1-methylethyl)oxy]phenyl}-1,2,4-oxadiazol-3-yl)-1,2,4,5-tetrahydro-3H-3-benzazepin-3-yl]ethyl}carbamate;
N-{2-[7-(5-{3-cyano-4-[(1-methylethyl)oxy]phenyl}-1,2,4-oxadiazol-3-yl)-1,2,4,5-tetrahydro-3H-3-benzazepin-3-yl]ethyl}acetamide;
methyl {3-[7-(5-{3-cyano-4-[(1-methylethyl)oxy]phenyl}-1,2,4-oxadiazol-3-yl)-1,2,4,5-tetrahydro-3H-3-benzazepin-3-yl]propyl}carbamate;
N-{3-[7-(5-{3-cyano-4-[(1-methylethyl)oxy]phenyl}-1,2,4-oxadiazol-3-yl)-1,2,4,5-tetrahydro-3H-3-benzazepin-3-yl]propyl}propanamide;
5-{3-[3-(2,3-dihydroxypropyl)-2,3,4,5-tetrahydro-1H-3-benzazepin-7-yl}-1,2,4-oxadiazol-5-yl]-2-(3-fluoro-1-azetidinyl)benzonitrile;
N-{2-[7-(5-{3-cyano-4-[(1-methylethyl)oxy]phenyl}-1,2,4-oxadiazol-3-yl)-1,2,4,5-tetrahydro-3H-3-benzazepin-3-yl]ethyl}-N′-ethylurea;
5-(3-{3-[(3-hydroxy-1-azetidinyl)acetyl]-2,3,4,5-tetrahydro-1H-3-benzazepin-7-yl}-1,2,4-oxadiazol-5-yl)-2-[(1-methylethyl)oxy]benzonitrile;
5-[3-(3-glycyl-2,3,4,5-tetrahydro-1H-3-benzazepin-7-yl}-1,2,4-oxadiazol-5-yl]-2-(propyloxy)-3-pyridinecarbonitrile;
5-(3-{3-[2-hydroxy-1-(hydroxymethyl)ethyl]-2,3,4,5-tetrahydro-1H-3-benzazepin-7-yl}-1,2,4-oxadiazol-5-yl)-2-[(1-methylethyl)amino]-3-pyridinecarbonitrile;
5-(3-{3-[2-hydroxy-1-(hydroxymethyl)ethyl]-2,3,4,5-tetrahydro-1H-3-benzazepin-7-yl}-1,2,4-oxadiazol-5-yl)-2-(propyloxy)-3-pyridinecarbonitrile;
2-(ethyloxy)-5-(3-{3-[2-hydroxy-1-(hydroxymethyl)ethyl]-2,3,4,5-tetrahydro-1H-3-benzazepin-7-yl}-1,2,4-oxadiazol-5-yl)-3-pyridinecarbonitrile;
2-[7-(5-{3-cyano-4-[(1-methylethyl)oxy]phenyl}-1,2,4-oxadiazol-3-yl)-1,2,4,5-tetrahydro-3H-3-benzazepin-3-yl]-N-(2-hydroxyethyl)acetamide;
5-[3-(3-glycyl-2,3,4,5-tetrahydro-1H-3-benzazepin-7-yl)-1,2,4-oxadiazol-5-yl]-2-[(1-methylethyl)amino]-3-pyridinecarbonitrile;
2-(ethyloxy)-5-[3-(3-glycyl-2,3,4,5-tetrahydro-1H-3-benzazepin-7-yl)-1,2,4-oxadiazol-5-yl]-3-pyridinecarbonitrile;
5-(3-{3-[(2S)-2,3-dihydroxypropyl]-2,3,4,5-tetrahydro-1H-3-benzazepin-7-yl}-1,2,4-oxadiazol-5-yl)-2-[(1-methylethyl)amino]-3-pyridinecarbonitrile;
5-(3-{3-[(2S)-2,3-dihydroxypropyl]-2,3,4,5-tetrahydro-1H-3-benzazepin-7-yl}-1,2,4-oxadiazol-5-yl)-2-(propyloxy)-3-pyridinecarbonitrile;
5-(3-{3-[(2S)-2,3-dihydroxypropyl]-2,3,4,5-tetrahydro-1H-3-benzazepin-7-yl}-1,2,4-oxadiazol-5-yl)-2-(ethyloxy)-3-pyridinecarbonitrile;
5-{3-[3-(2-methylalanyl)-2,3,4,5-tetrahydro-1H-3-benzazepin-7-yl]-1,2,4-oxadiazol-5-yl}-2-[(1-methylethyl)oxy]benzonitrile;
2-[6-(5-{3-cyano-4-[(1-methylethyl)oxy]phenyl}-1,2,4-oxadiazol-3-yl)-5-methyl-3,4-dihydro-2(1H)-isoquinolinyl]-N-(1R)-2-hydroxy-1-methylethyl]acetamide;
2-[6-(5-{3-cyano-4-[(1-methylethyl)oxy]phenyl}-1,2,4-oxadiazol-3-yl)-5-methyl-3,4-dihydro-2(1H)-isoquinolinyl]-N-[(1S)-2-hydroxy-1-methylethyl]acetamide;
2-[6-(5-{3-cyano-4-[(1-methylethyl)oxy]phenyl}-1,2,4-oxadiazol-3-yl)-5-methyl-3,4-dihydro-2(1H)-isoquinolinyl]-N-[(2R)-2-hydroxypropyl]acetamide;
2-[6-(5-{3-cyano-4-[(1-methylethyl)oxy]phenyl}-1,2,4-oxadiazol-3-yl)-5-methyl-3,4-dihydro-2(1H)-isoquinolinyl]-N-[(2S)-2-hydroxypropyl]acetamide;
2-[6-(5-{3-cyano-4-[(1-methylethyl)oxy]phenyl}-1,2,4-oxadiazol-3-yl)-5-methyl-3,4-dihydro-2(1H)-isoquinolinyl]-N-(2-hydroxyethyl)acetamide;
5-(3-{2-[2-(3-hydroxy-1-azetidinyl)-2-oxoethyl]-5-methyl-1,2,3,4-tetrahydro-6-isoquinolinyl}-1,2,4-oxadiazol-5-yl)-2-[(1-methylethyl)oxy]benzonitrile;
2-[6-(5-{3-cyano-4-[(1-methylethyl)oxy]phenyl}-1,2,4-oxadiazol-3-yl)-5-methyl-3,4-dihydro-2(1H)-isoquinolinyl]-N-[(2S)-2,3-dihydroxypropyl]acetamide;
2-[6-(5-{3-cyano-4-[(1-methylethyl)oxy]phenyl}-1,2,4-oxadiazol-3-yl)-5-methyl-3,4-dihydro-2(1H)-isoquinolinyl]-N-[2-hydroxy-1-(hydroxymethyl)ethyl]acetamide;
5-[3-(2-{N-[(2S)-2,3-dihydroxypropyl]glycyl}-5-methyl-1,2,3,4-tetrahydro-6-isoquinolinyl)-1,2,4-oxadiazol-5-yl]-2-[(1-methylethyl)oxy]benzonitrile;
5-[3-(2-{N-[2-hydroxy-1-(hydroxymethyl)ethyl]glycyl}-5-methyl-1,2,3,4-tetrahydro-6-isoquinolinyl)-1,2,4-oxadiazol-5-yl]-2-[(1-methylethyl)oxy]benzonitrile;
5-[3-(2-{N-(1R)-2-hydroxy-1-methylethyl]glycyl}-5-methyl-1,2,3,4-tetrahydro-6-isoquinolinyl)-1,2,4-oxadiazol-5-yl]-2-[(1-methylethyl)oxy]benzonitrile;
5-[3-(2-{N-[(2R)-2-hydroxypropyl]glycyl}-5-methyl-1,2,3,4-tetrahydro-6-isoquinolinyl)-1,2,4-oxadiazol-5-yl]-2-[(1-methylethyl)oxy]benzonitrile;
5-[3-(2-{N-[(1S)-2-hydroxy-1-methylethyl]glycyl}-5-methyl-1,2,3,4-tetrahydro-6-isoquinolinyl)-1,2,4-oxadiazol-5-yl]-2-[(1-methylethyl)oxy]benzonitrile;
5-(3-{2-[N-(2-hydroxyethyl)glycyl]-5-methyl-1,2,3,4-tetrahydro-6-isoquinolinyl}-1,2,4-oxadiazol-5-yl)-2-[(1-methylethyl)oxy]benzonitrile;
5-(3-{2-[(2S)-2,3-dihydroxypropyl]-5-methyl-1,2,3,4-tetrahydro-6-isoquinolinyl}-1,2,4-oxadiazol-5-yl)-2-[(1-methylethyl)oxy]-3-pyridinecarbonitrile;
5-(3-{2-[(2R)-2,3-dihydroxypropyl]-5-methyl-1,2,3,4-tetrahydro-6-isoquinolinyl}-1,2,4-oxadiazol-5-yl)-2-[(1-methylethyl)oxy]-3-pyridinecarbonitrile;
5-(3-{2-[2-hydroxy-1-(hydroxymethyl)ethyl]-5-methyl-1,2,3,4-tetrahydro-6-isoquinolinyl}-1,2,4-oxadiazol-5-yl)-2-[(1-methylethyl)oxy]-3-pyridinecarbonitrile;
5-(3-{3-[(2S)-2,3-dihydroxypropyl]-2,3,4,5-tetrahydro-1H-3-benzazepin-7-yl}-1,2,4-oxadiazol-5-yl)-2-[(2,2,2-trifluoroethyl)oxy]benzonitrile;
2-[6-(5-{3-chloro-4-[(1-methylethyl)oxy]phenyl}-1,2,4-oxadiazol-3-yl)-5-methyl-3,4-dihydro-2(1H)-isoquinolinyl]-N-(2-hydroxyethyl)acetamide;
5-[3-(2-glycyl-5-methyl-1,2,3,4-tetrahydro-6-isoquinolinyl)-1,2,4-oxadiazol-5-yl]-2-[(1-methylethyl)oxy]-3-pyridinecarbonitrile;
5-(3-{3-[(2S)-2,3-dihydroxypropyl]-8-methyl-2,3,4,5-tetrahydro-1H-3-benzazepin-7-yl}-1,2,4-oxadiazol-5-yl)-2-[(1-methylethyl)oxy]benzonitrile;
2-({2-[6-(5-{3-chloro-4-[(1-methylethyl)oxy]phenyl}-1,2,4-oxadiazol-3-yl)-5-methyl-3,4-dihydro-2(1H)-isoquinolinyl]-2-oxoethyl}amino)ethanol;
5-[3-(3-glycyl-6-methyl-2,3,4,5-tetrahydro-1H-3-benzazepin-7-yl)-1,2,4-oxadiazol-5-yl]-2-[(1-methylethyl)oxy]benzonitrile;
{2-[6-(5-{5-chloro-6-[(1-methylethyl)oxy]-3-pyridinyl}-1,2,4-oxadiazol-3-yl)-5-methyl-3,4-dihydro-2(1H)-isoquinolinyl]-2-oxoethyl}amine;
5-(3-{3-[(2S)-2,3-dihydroxypropyl]-2,3,4,5-tetrahydro-1H-3-benzazepin-7-yl}-1,2,4-oxadiazol-5-yl)-2-(propylamino)-3-pyridinecarbonitrile;
5-(3-{3-[(2S)-2,3-dihydroxypropyl]-2,3,4,5-tetrahydro-1H-3-benzazepin-7-yl}-1,2,4-oxadiazol-5-yl)-2-(propylamino)-3-pyridinecarbonitrile;
2-[6-(5-{5-chloro-6-[(1-methylethyl)oxy]-3-pyridinyl}-1,2,4-oxadiazol-3-yl)-5-methyl-3,4-dihydro-2(1H)-isoquinolinyl]-1,3-propanediol;
5-(3-{3-[(2S)-2,3-dihydroxypropyl]-6-methyl-2,3,4,5-tetrahydro-1H-3-benzazepin-7-yl}-1,2,4-oxadiazol-5-yl)-2-(propylamino)-3-pyridinecarbonitrile;
2-[7-(5-{3-cyano-4-[(1-methylethyl)oxy]phenyl}-1,2,4-oxadiazol-3-yl)-6-methyl-1,2,4,5-tetrahydro-3H-3-benzazepin-3-yl]-N-(2-hydroxyethyl)acetamide;
2-[7-(5-{3-cyano-4-[(1-methylethyl)oxy]phenyl}-1,2,4-oxadiazol-3-yl)-6-methyl-1,2,4,5-tetrahydro-3H-3-benzazepin-3-yl]-N-[(2R)-2-hydroxypropyl]acetamide;
2-[7-(5-{3-cyano-4-[(1-methylethyl)oxy]phenyl}-1,2,4-oxadiazol-3-yl)-6-methyl-1,2,4,5-tetrahydro-3H-3-benzazepin-3-yl]-N-[(2S)-2-hydroxypropyl]acetamide;
5-(3-{3-[2-hydroxy-1-(hydroxymethyl)ethyl]-6-methyl-2,3,4,5-tetrahydro-1H-3-benzazepin-7-yl}-1,2,4-oxadiazol-5-yl)-2-(propylamino)-3-pyridinecarbonitrile;
5-(3-{3-[2-hydroxy-1-(hydroxymethyl)ethyl]-2,3,4,5-tetrahydro-1H-3-benzazepin-7-yl}-1,2,4-oxadiazol-5-yl)-2-(propylamino)-3-pyridinecarbonitrile;
5-[3-(3-glycyl-2,3,4,5-tetrahydro-1H-3-benzazepin-7-yl)-1,2,4-oxadiazol-5-yl]-2-(propylamino)-3-pyridinecarbonitrile;
5-[3-(3-glycyl-2,3,4,5-tetrahydro-1H-3-benzazepin-7-yl)-1,2,4-oxadiazol-5-yl]-2-(propylamino)-3-pyridinecarbonitrile;
5-(3-{3-[(2S)-2,3-dihydroxypropyl]-6-methyl-2,3,4,5-tetrahydro-1H-3-benzazepin-7-yl}-1,2,4-oxadiazol-5-yl)-2-[(1-methylethyl)oxy]benzonitrile;
5-(3-{3-[(2R)-2,3-dihydroxypropyl]-6-methyl-2,3,4,5-tetrahydro-1H-3-benzazepin-7-yl}-1,2,4-oxadiazol-5-yl)-2-[(1-methylethyl)oxy]benzonitrile;
5-(3-{3-[N-(2-hydroxyethyl)glycyl]-6-methyl-2,3,4,5-tetrahydro-1H-3-benzazepin-7-yl}-1,2,4-oxadiazol-5-yl)-2-[(1-methylethyl)oxy]benzonitrile;
5-(3-{3-[2-hydroxy-1-(hydroxymethyl)ethyl]-6-methyl-2,3,4,5-tetrahydro-1H-3-benzazepin-7-yl}-1,2,4-oxadiazol-5-yl)-2-[(1-methylethyl)oxy]benzonitrile;
2-[7-(5-{3-cyano-4-[(1-methylethyl)oxy]phenyl}-1,2,4-oxadiazol-3-yl)-1,2,4,5-tetrahydro-3H-3-benzazepin-3-yl]-N-(3-hydroxypropyl)acetamide;
2-[7-(5-{3-cyano-4-[(1-methylethyl)oxy]phenyl}-1,2,4-oxadiazol-3-yl)-6-methyl-1,2,4,5-tetrahydro-3H-3-benzazepin-3-yl]-N-[(1S)-2-hydroxy-1-methylethyl]acetamide;
2-[7-(5-{3-cyano-4-[(1-methylethyl)oxy]phenyl}-1,2,4-oxadiazol-3-yl)-6-methyl-1,2,4,5-tetrahydro-3H-3-benzazepin-3-yl]-N-(1R)-2-hydroxy-1-methylethyl]acetamide;
2-[7-(5-{3-cyano-4-[(1-methylethyl)oxy]phenyl}-1,2,4-oxadiazol-3-yl)-1,2,4,5-tetrahydro-3H-3-benzazepin-3-yl]-N-(2-hydroxyethyl)-2-methylpropanamide;
2-[7-(5-{3-cyano-4-[(1-methylethyl)oxy]phenyl}-1,2,4-oxadiazol-3-yl)-1,2,4,5-tetrahydro-3H-3-benzazepin-3-yl]-N-(2-hydroxyethyl)propanamide;
2-[7-(5-{3-cyano-4-[(1-methylethyl)oxy]phenyl}-1,2,4-oxadiazol-3-yl)-1,2,4,5-tetrahydro-3H-3-benzazepin-3-yl]-N-(1R)-2-hydroxy-1-methylethyl]propanamide;
2-[7-(5-{3-cyano-4-[(1-methylethyl)oxy]phenyl}-1,2,4-oxadiazol-3-yl)-1,2,4,5-tetrahydro-3H-3-benzazepin-3-yl]-N-(1R)-2-hydroxy-1-methylethyl]-2-methylpropanamide;
(2R)-3-[7-(5-{3-cyano-4-[(1-methylethyl)oxy]phenyl}-1,2,4-oxadiazol-3-yl)-1,2,4,5-tetrahydro-3H-3-benzazepin-3-yl]-2-hydroxy-N-(2-hydroxyethyl)propanamide;
(2R)-3-[7-(5-{3-cyano-4-[(1-methylethyl)oxy]phenyl}-1,2,4-oxadiazol-3-yl)-1,2,4,5-tetrahydro-3H-3-benzazepin-3-yl]-2-hydroxy-N-(1R)-2-hydroxy-1-methylethyl]propanamide;
5-(3-{2-[(2R)-2,3-dihydroxypropyl]-1,2,3,4-tetrahydro-6-isoquinolinyl}-1,2,4-oxadiazol-5-yl)-2-[(1-methylethyl)oxy]-3-pyridinecarbonitrile;
5-(3-{2-[(2S)-2,3-dihydroxypropyl]-1,2,3,4-tetrahydro-6-isoquinolinyl}-1,2,4-oxadiazol-5-yl)-2-[(1-methylethyl)oxy]-3-pyridinecarbonitrile;
5-(3-{2-[2-hydroxy-1-(hydroxymethyl)ethyl]-1,2,3,4-tetrahydro-6-isoquinolinyl}-1,2,4-oxadiazol-5-yl)-2-[(1-methylethyl)oxy]-3-pyridinecarbonitrile;
2-[6-(5-{3-cyano-4-[(1-methylethyl)oxy]phenyl}-1,2,4-oxadiazol-3-yl)-3,4-dihydro-2(1H)-isoquinolinyl}-N-[(1S)-2-hydroxy-1-methylethyl]acetamide;
2-[6-(5-{5-cyano-6-[(1-methylethyl)oxy]-3-pyridinyl}-1,2,4-oxadiazol-3-yl)-5-methyl-3,4-dihydro-2(1H)isoquinolinyl]-N-[(1S)-2-hydroxy-1-methylethyl]acetamide;
5-(3-{3-[(2R)-2,3-dihydroxypropyl]-2,3,4,5-tetrahydro-1H-3-benzazepin-7-yl}-1,2,4-oxadiazol-5-yl)-2-[(1-methylethyl)amino]-3-pyridinecarbonitrile;
5-(3-{3-[(2R)-2,3-dihydroxypropyl]-6-methyl-2,3,4,5-tetrahydro-1H-3-benzazepin-7-yl}-1,2,4-oxadiazol-5-yl)-2-[(1-methylethyl)amino]-3-pyridinecarbonitrile;
5-(3-{3-[(2S)-2,3-dihydroxypropyl]-6-methyl-2,3,4,5-tetrahydro-1H-3-benzazepin-7-yl}-1,2,4-oxadiazol-5-yl)-2-[(1-methylethyl)amino]-3-pyridinecarbonitrile;
5-(3-{2-[(2R)-2,3-dihydroxypropyl]-5-methyl-1,2,3,4-tetrahydro-6-isoquinolinyl}-1,2,4-oxadiazol-5-yl)-2-[(1-methylethyl)amino]-3-pyridinecarbonitrile;
5-(3-{3-[2-hydroxy-1-(hydroxymethyl)ethyl]-6-methyl-2,3,4,5-tetrahydro-1H-3-benzazepin-7-yl}-1,2,4-oxadiazol-5-yl)-2-[(1-methylethyl)amino]-3-pyridinecarbonitrile
5-(3-{2-[2-hydroxy-1-(hydroxymethyl)ethyl]-5-methyl-1,2,3,4-tetrahydro-6-isoquinolinyl}-1,2,4-oxadiazol-5-yl)-2-[(1-methylethyl)amino]-3-pyridinecarbonitrile;
5-(3-{2-[(2S)-2,3-dihydroxypropyl]-5-methyl-1,2,3,4-tetrahydro-6-isoquinolinyl}-1,2,4-oxadiazol-5-yl)-2-[(1-methylethyl)amino]-3-pyridinecarbonitrile;
5-(3-{2-[(2R)-2,3-dihydroxypropyl]-1,2,3,4-tetrahydro-5-isoquinolinyl}-1,2,4-oxadiazol-5-yl)-2-[(1-methylethyl)oxy]benzonitrile;
5-(3-{2-[(2R)-2,3-dihydroxypropyl]-1,2,3,4-tetrahydro-5-isoquinolinyl}-1,2,4-oxadiazol-5-yl)-2-[(1-methylethyl)oxy]benzonitrile;
5-(3-{2-[2-hydroxy-1-(hydroxymethyl)ethyl]-1,2,3,4-tetrahydro-5-isoquinolinyl}-1,2,4-oxadiazol-5-yl)-2-[(1-methylethyl)oxy]benzonitrile;
5-(5-{3-[(2R)-2,3-dihydroxypropyl]-2,3,4,5-tetrahydro-1H-3-benzazepin-7-yl}-1,3,4-thiadiazol-2-yl)-2-[(1-methylethyl)oxy]benzonitrile;
5-(5-{3-[(2S)-2,3-dihydroxypropyl]-2,3,4,5-tetrahydro-1H-3-benzazepin-7-yl}-1,3,4-thiadiazol-2-yl)-2-[(1-methylethyl)oxy]benzonitrile;
2-[7-(5-{3-cyano-4-[(1-methylethyl)oxy]phenyl}-1,2,4-oxadiazol-3-yl)-3,4-dihydro-2(1H)-isoquinolinyl}-N-[(1S)-2-hydroxy-1-methylethyl]acetamide;
5-(5-{2-[2-hydroxy-1-(hydroxymethyl)ethyl]-5-methyl-1,2,3,4-tetrahydro-6-isoquinolinyl}-1,3,4-thiadiazol-2-yl)-2-[(1-methylethyl)oxy]benzonitrile;
2-[7-(5-{3-cyano-4-[(1-methylethyl)oxy]phenyl}-1,2,4-oxadiazol-3-yl)-1,2,4,5-tetrahydro-3H-3-benzazepin-3-yl]-N-(3,3,3-trifluoro-2-hydroxypropy))acetamide;
2-[7-(5-{3-cyano-4-[(1-methylethyl)oxy]phenyl}-1,2,4-oxadiazol-3-yl)-6-methyl-1,2,4,5-tetrahydro-3H-3-benzazepin-3-yl]-N-[2-hydroxy-1-(hydroxymethyl)ethyl)acetamide;
5-(3-{3-[N-(2,3-dihydroxypropyl)glycyl]-6-methyl-2,3,4,5-tetrahydro-1H-3-benzazepin-7-yl}-1,2,4-oxadiazol-5-yl)-2-[(1-methylethyl)oxy]benzonitrile;
5-(3-{3-[N-(2,3-dihydroxypropyl)glycyl]-8-methyl-2,3,4,5-tetrahydro-1H-3-benzazepin-7-yl}-1,2,4-oxadiazol-5-yl)-2-[(1-methylethyl)oxy]benzonitrile;
5-[3-(3-{N-[2-hydroxy-1-(hydroxymethyl)ethyl]glycyl}-6-methyl-2,3,4,5-tetrahydro-1H-3-benzazepin-7-yl)-1,2,4-oxadiazol-5-yl]-2-[(1-methylethyl)oxy]benzonitrile;
2-({2-[6-(5-{3-chloro-4-[(1-methylethyl)oxy]phenyl}-1,2,4-oxadiazol-3-yl)-5-methyl-3,4-dihydro-2(1H)-isoquinolinyl]-2-oxoethyl}amino)-1,3-propanediol,
5-[3-(3-glycyl-2,3,4,5-tetrahydro-1H-3-benzazepin-7-yl]-1,2,4-oxadiazol-5-yl]-2-[(2,2,2-trifluoroethyl)oxy]benzonitrile,
2-[6-(5-{3-chloro-4-[(1-methylethyl)oxy]phenyl}-1,2,4-oxadiazol-3-yl)-5-methyl-3,4-dihydro-2(1H)-isoquinolinyl]-N-[(2S)-2,3-dihydroxypropyl]acetamide;
2-[6-(5-{3-chloro-4-[(1-methylethyl)oxy]phenyl}-1,2,4-oxadiazol-3-yl)-5-methyl-3,4-dihydro-2(1H)-isoquinolinyl]-N-[2-hydroxy-1-(hydroxymethyl)ethyl)acetamide,
2-[7-(5-{3-cyano-4-[(1-methylethyl)oxy]phenyl}-1,2,4-oxadiazol-3-yl)-1,2,4,5-tetrahydro-3H-3-benzazepin-3-yl]-N-[(2S)-2,3-dihydroxypropyl]acetamide,
2-[7-(5-{3-cyano-4-[(1-methylethyl)oxy]phenyl}-1,2,4-oxadiazol-3-yl)-1,2,4,5-tetrahydro-3H-3-benzazepin-3-yl]-N-[2-hydroxy-1-(hydroxymethyl)ethyl)acetamide,
5-[3-(3-{N-[(2S)-2,3-dihydroxypropyl]glycyl}-2,3,4,5-tetrahydro-1H-3-benzazepin-7-yl)-1,2,4-oxadiazol-5-yl]-2-[(1-methylethyl)oxy]benzonitrile hydrochloride,
5-[3-(3-{N-[2-hydroxy-1-(hydroxymethyl)ethyl]glycyl}-2,3,4,5-tetrahydro-1H-3-benzazepin-7-yl)-1,2,4-oxadiazol-5-yl]-2-[(1-methylethyl)oxy]benzonitrile hydrochloride;
5-[3-(2-{N-[(2S)-2-hydroxypropyl]glycyl}-5-methyl-1,2,3,4-tetrahydro-6-isoquinolinyl)-1,2,4-oxadiazol-5-yl]-2-[(1-methylethyl)oxy]benzonitrile hydrochloride;
5-(5-{3-[2-hydroxy-1-(hydroxymethyl)ethyl]-2,3,4,5-tetrahydro-1H-3-benzazepin-7-yl}-1,3,4-thiadiazol-2-yl)-2-[(1-methylethyl)oxy]benzonitrile;
5-(5-{3-[N-(2-hydroxyethyl)glycyl]-2,3,4,5-tetrahydro-1H-3-benzazepin-7-yl}-1,3,4-thiadiazol-2-yl)-2-[(1-methylethyl)oxy]benzonitrile;
5-(5-{2-[N-(2-hydroxyethyl)glycyl]-5-methyl-1,2,3,4-tetrahydro-6-isoquinolinyl}-1,3,4-thiadiazol-2-yl)-2-[(1-methylethyl)oxy]benzonitrile;
2-({2-[6-(5-{3-chloro-4-[(1-methylethyl)oxy]phenyl}-1,3,4-thiadiazol-2-yl)-5-methyl-3,4-dihydro-2(1H)-isoquinolinyl]-2-oxoethyl}amino)ethanol; and
2-[6-(5-{3-chloro-4-[(1-methylethyl)oxy]phenyl}-1,3,4-thiadiazol-2-yl)-5-methyl-3,4-dihydro-2(1H)-isoquinolinyl]-1,3-propanediol,
or a salt thereof.

4. A compound according to claim 1, which is 5-(3-{2-[2-hydroxy-1-(hydroxymethyl)ethyl]-5-methyl-1,2,3,4-tetrahydro-6-isoquinolinyl}-1,2,4-oxadiazol-5-yl)-2-[(1-methylethyl)oxy]benzonitrile or a pharmaceutically acceptable salt thereof.

5. A compound according to claim 1, which is 5-(3-{2-[2-hydroxy-1-(hydroxymethyl)ethyl]-5-methyl-1,2,3,4-tetrahydro-6-isoquinolinyl}-1,2,4-oxadiazol-5-yl)-2-[(1-methylethyl)oxy]-3-pyridinecarbonitrile or a pharmaceutically acceptable salt thereof.

6. A compound according to claim 1, which is 5-(5-{2-[2-hydroxy-1-(hydroxymethyl)ethyl]-5-methyl-1,2,3,4-tetrahydro-6-isoquinolinyl}-1,3,4-thiadiazol-2-yl)-2-[(1-methylethyl)oxy]benzonitrile or a pharmaceutically acceptable salt thereof.

7. A method of treating a condition or disorder mediated by S1P1 comprising administering to a subject with said condition or disorder a therapeutically effective amount of a compound of formula (I) or a pharmaceutically acceptable salt thereof according to claim 1.

8. A method according to claim 7, wherein the condition or disorder is multiple sclerosis, autoimmune diseases, chronic inflammatory disorders, asthma, inflammatory neuropathies, arthritis, transplantation, Crohn's disease, ulcerative colitis, lupus erythematosis, psoriasis, ischemia-reperfusion injury, solid tumours, and tumour metastasis, diseases associated with angiogenesis, vascular diseases, pain conditions, acute viral diseases, inflammatory bowel conditions, insulin and non-insulin dependant diabetes.

9. A method according to claim 8, wherein the condition is psoriasis.

10. (canceled)

11. Use according to claim 9, wherein the condition or disorder multiple sclerosis, autoimmune diseases, chronic inflammatory disorders, asthma, inflammatory neuropathies, arthritis, transplantation, Crohn's disease, ulcerative colitis, lupus erythematosis, psoriasis, ischemia-reperfusion injury, solid tumours, and tumour metastasis, diseases associated with angiogenesis, vascular diseases, pain conditions, acute viral diseases, inflammatory bowel conditions, insulin and non-insulin dependant diabetes.

12. Use according to claim 11, wherein the condition is psoriasis.

13. A pharmaceutical composition comprising a compound of formula (I) or a pharmaceutically acceptable salt thereof according to claim 1.

14. (canceled)

15. (canceled)
